(19) United States
(12) Patent Application Publication
(10) Pub. No.: US 2025/0282736 A1
(43) Pub. Date: Sep. 11, 2025

(54) SMALL MOLECULE MODULATORS OF HUMAN PREGNANE X RECEPTOR

(71) Applicant: ST. JUDE CHILDREN'S RESEARCH HOSPITAL, Memphis, TN (US)

(72) Inventors: Taosheng Chen, Germantown, TN (US); Yongtao Li, Memphis, TN (US); Wenwei Lin, Memphis, TN (US)

(21) Appl. No.: 18/858,952

(22) PCT Filed: Apr. 20, 2023
(86) PCT No.: PCT/US2023/019279

§ 371 Date: Oct. 22, 2024

Related U.S. Application Data
(60) Provisional application No. 63/333,929, filed on Apr. 22, 2022.

(51) Int. Cl. C07D 249/06; C07D 401/04; C07D 401/12; C07D 403/12; C07D 405/04; C07F 5/02

(52) U.S. Cl. CPC C07D 249/06; C07D 401/04; C07D 401/12; C07D 403/12; C07D 405/04; C07F 5/025

(57) ABSTRACT

The present disclosure relates to 1,4,5-substituted 1,2,3-triazoles useful as modulators of pregnane X receptor (PXR) and in the treatment of disorders associated with PXR dysfunction (e.g., disorders of uncontrolled cellular proliferation, bowel disorders). The invention further relates to uses of the disclosed compounds in decreasing adverse drug reactions such as, for example, adverse reactions associated with administration of an anticancer agent, an antibacterial agent, a non-steroidal anti-inflammatory agent, and an anticonvulsant agent. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/333,929, filed on Apr. 22, 2022, the contents of which are incorporated herein by reference in their entirety.

### STATEMENT REGARDING FEDERALLY SPONSORED RESEARCH

This invention was made with government support under grant number GM118041, awarded by the National Institutes of Health. The government has certain rights in the invention.

### BACKGROUND

The pregnane X receptor (PXR) regulates the metabolism and excretion of xenobiotics and endobiotics by regulating the expression of drug-metabolizing enzymes and drug transporters. By affecting drug metabolism, changes in the expression levels of PXR target genes can influence the therapeutic and toxicologic response to drugs and cause adverse drug-drug interactions. The activity of PXR is largely regulated by direct ligand binding, and the unique structure of PXR allows the binding of a variety of drugs and prospective drugs. That is, a drug or prospective drug molecule can directly modulate the activity of PXR. As such, PXR is associated with multiple undesired drug-drug interactions.

The promiscuous activation of PXR by natural substances and xenobiotics has been implicated as a mechanism that accelerates the metabolism of affected drugs, which leads to unanticipated adverse drug reactions and lack of efficacy. For example, PXR activation in the gut can lower drug bioavailability. Recent data also supports a major role for PXR activation in controlling (or decreasing) the transport of drugs across the blood brain barrier, underscoring PXR's role in drug delivery to the brain and limiting the effectiveness of therapeutics on primary brain tumors or brain metastases. Therefore, in the context of cancer therapeutics, controlling PXR activation serves to improve both drug metabolism and delivery. Thus, there is a need for additional understanding of how PXR activation and inhibition can benefit treatment of various diseases. There is also a need for non-toxic selective modulators of PXR. These and other needs are satisfied by the present disclosure.

### SUMMARY

In accordance with the purpose(s) of the invention, as embodied and broadly described herein, the invention, in one aspect, relates to 1,4,5-substituted 1,2,3-triazoles that are useful as modulators of pregnane X receptor (PXR) and in the treatment of disorders associated with PXR dysfunction (e.g., cancer, bowel disorders). The invention further relates to the use of the disclosed compounds in decreasing adverse drug reactions such as, for example, adverse drug reactions associated with administration of an anticancer agent, an antibacterial agent, a non-steroidal anti-inflammatory agent, or an anticonvulsant agent.

Disclosed are compounds having a structure represented by a formula:

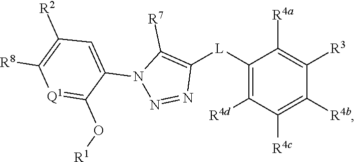

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is hydrogen, halogen, C1-C8 alkyl, C1-C8 haloalkyl, C1-C8 alkoxy, C1-C8 haloalkoxy, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

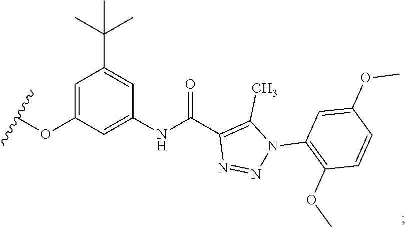

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R⁸ is selected from hydrogen, halogen, and C1-C4 alkoxy; wherein R⁷ is selected from hydrogen and C1-C4 alkyl; provided that when L is —C(O)NR¹⁰—, then R² is selected from C1-C4 haloalkyl, C1-C4 alkoxy, and C1-C4 haloalkoxy, and R³ is C1-C8 alkyl, —CO₂(C1-C4 alkyl), or —C(O)Cy², or a pharmaceutically acceptable salt thereof.

Also disclosed are compounds having a structure represented by a formula:

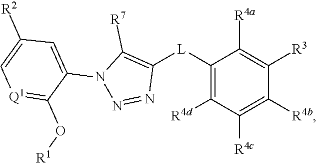

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is selected from hydrogen, halogen, C1-C8 alkyl, C1-C8 haloalkyl, C1-C8 alkoxy, and C1-C8 haloalkoxy; and wherein each of R^{4a}, R^{4b}, R^{4c}, and R^{4d} is independently selected from hydrogen, halogen, —CN, —NH₂, OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

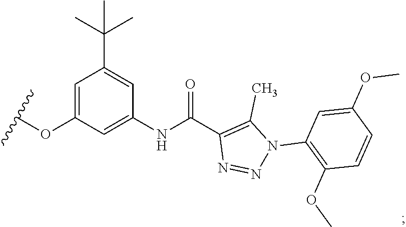

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein R⁷ is selected from hydrogen and C1-C4 alkyl; and wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl), provided that when L is —C(O)NR¹⁰—, then R² is selected from C1-C4 haloalkyl, C1-C4 alkoxy, and C1-C4 haloalkoxy, and R³ is C1-C8 alkyl, or a pharmaceutically acceptable salt thereof.

Also disclosed are compounds selected from:

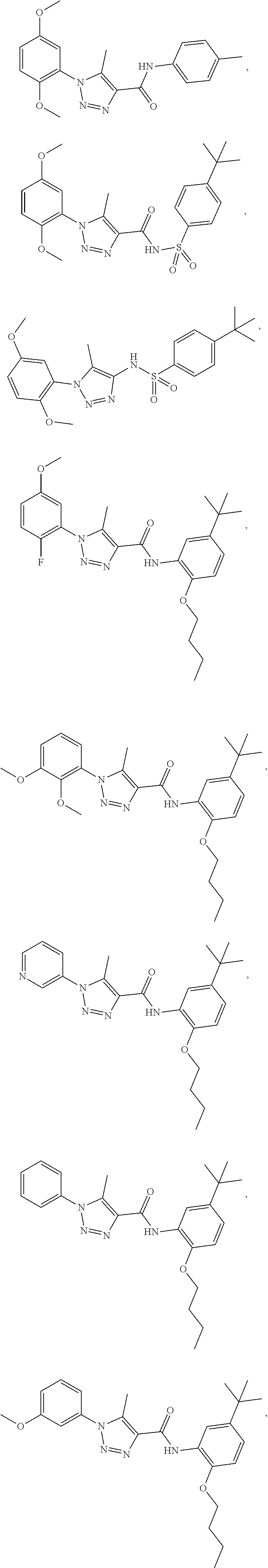

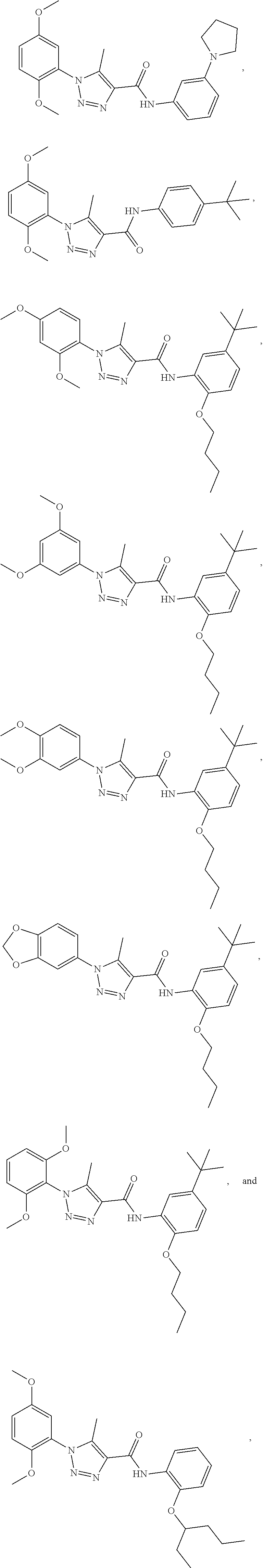

or a pharmaceutically acceptable salt thereof.

Also disclosed are pharmaceutical compositions comprising a therapeutically effective amount of a disclosed compound or a pharmaceutically acceptable salt thereof and a pharmaceutically acceptable carrier.

Also disclosed are pharmaceutical compositions comprising a therapeutically effective amount of a compound selected from:

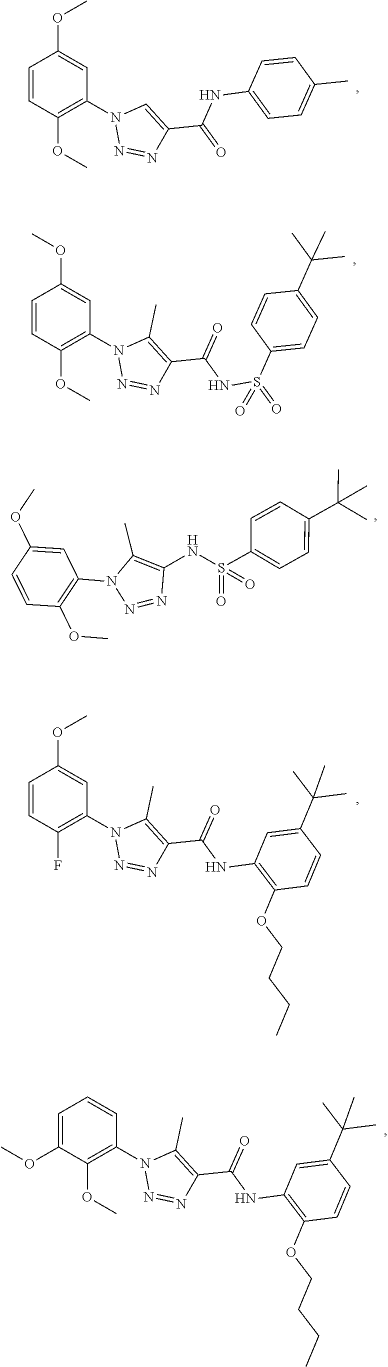

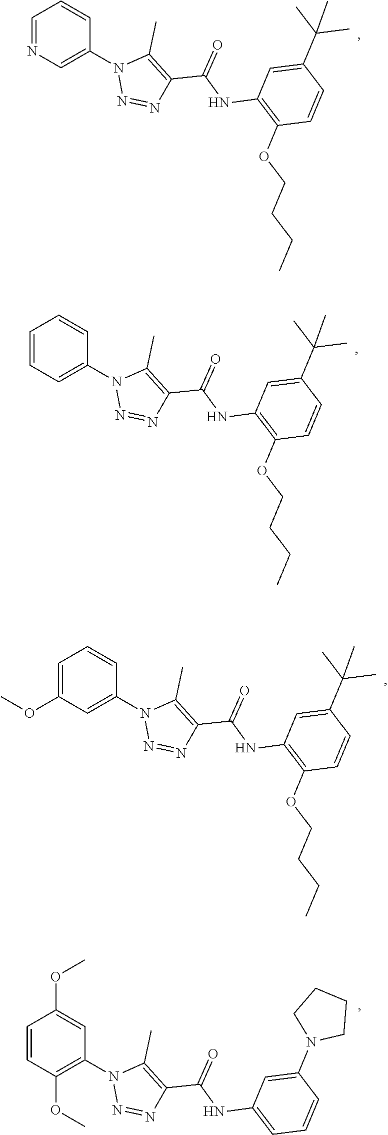

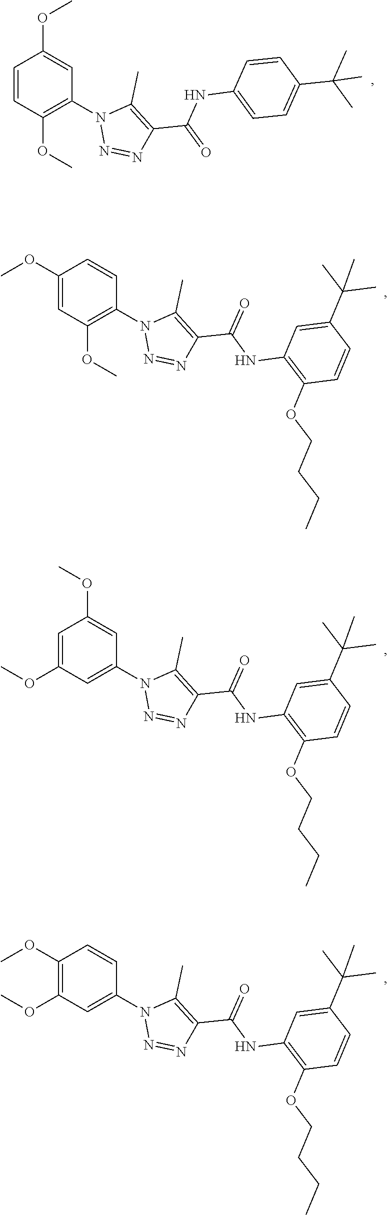

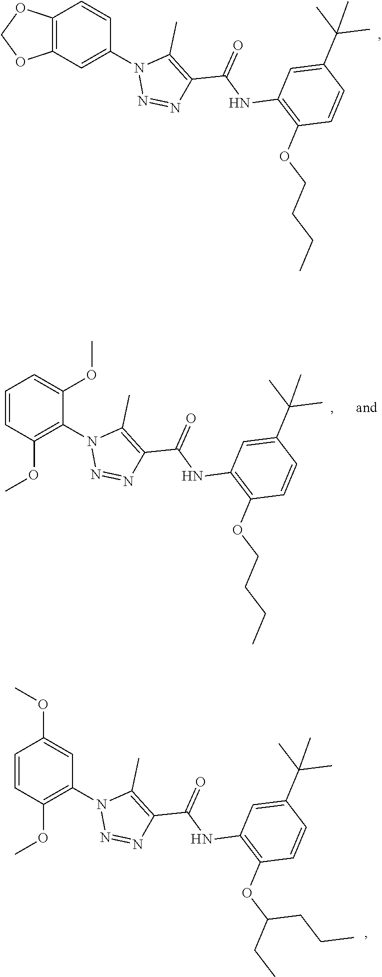 [SMILES: CCCCOc1ccc(C(C)(C)C)cc1NC(=O)c1nnn(-c2ccc3c(c2)OCO3)c1C ; CCCCOc1ccc(C(C)(C)C)cc1NC(=O)c1nnn(-c2c(OC)cccc2OC)c1C ; CCCC(CC)Oc1ccc(C(C)(C)C)cc1NC(=O)c1nnn(-c2cc(OC)ccc2OC)c1C]

or a pharmaceutically acceptable salt thereof, and a pharmaceutically acceptable carrier.

Also disclosed are methods for modulating pregnane X receptor (PXR) activity in a subject in need thereof, the method comprising administering to the subject an effective amount of a disclosed compound or a pharmaceutically acceptable salt thereof.

Also disclosed are methods for modulating pregnane X receptor (PXR) activity in a subject in need thereof, the method comprising administering to the subject an effective amount of a compound selected from:

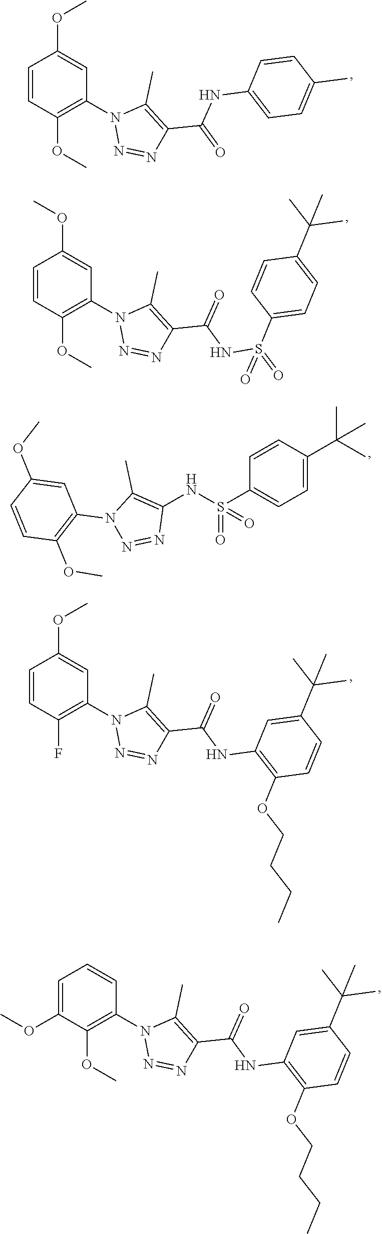

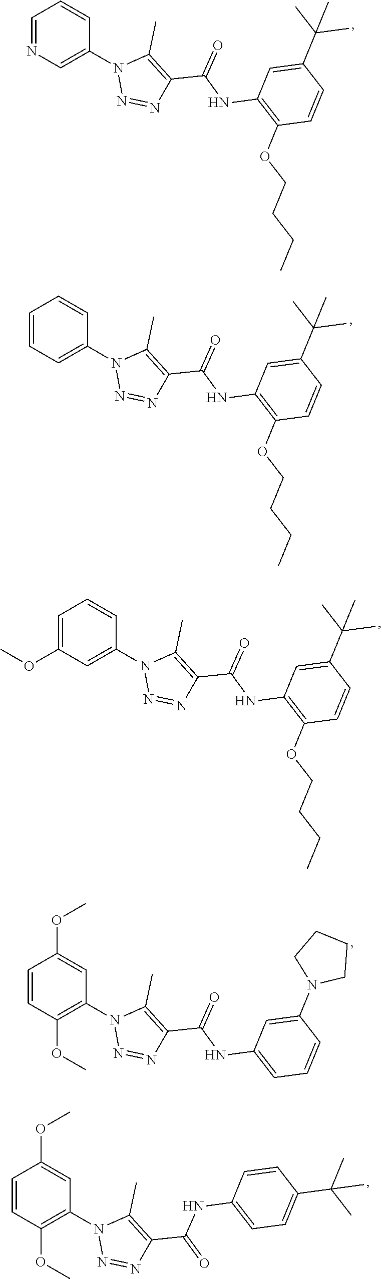

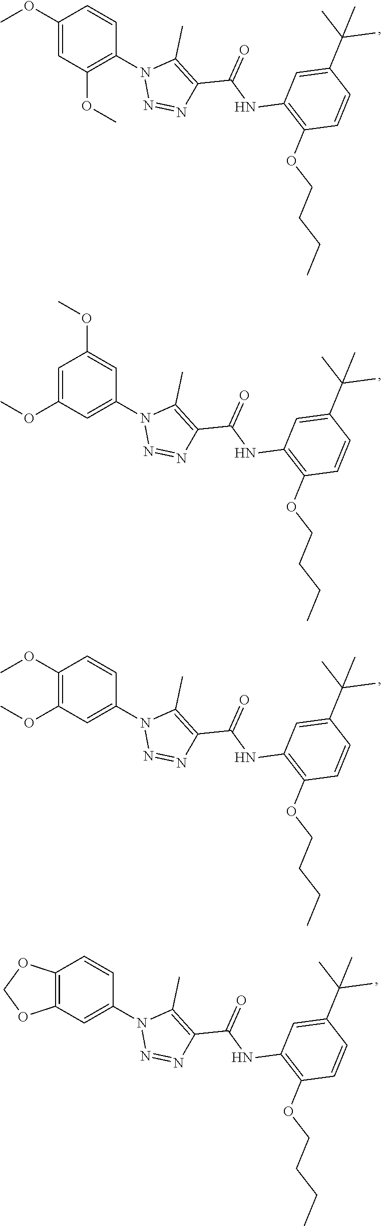

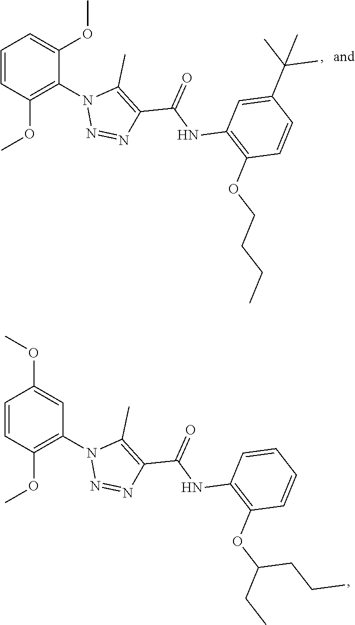 [SMILES: CCCCOc1ccc(C(C)(C)C)cc1NC(=O)c1nnn(-c2c(OC)cccc2OC)c1C ; CCCC(CC)Oc1ccccc1NC(=O)c1nnn(-c2cc(OC)ccc2OC)c1C]

or a pharmaceutically acceptable salt thereof.

Also disclosed are methods for modulating pregnane X receptor (PXR) activity in a cell, the method comprising contacting the cell with an effective amount of the disclosed compound or a pharmaceutically acceptable salt thereof.

Also disclosed are methods for modulating pregnane X receptor (PXR) activity in a cell, the method comprising contacting the cell with an effective amount a compound selected from:

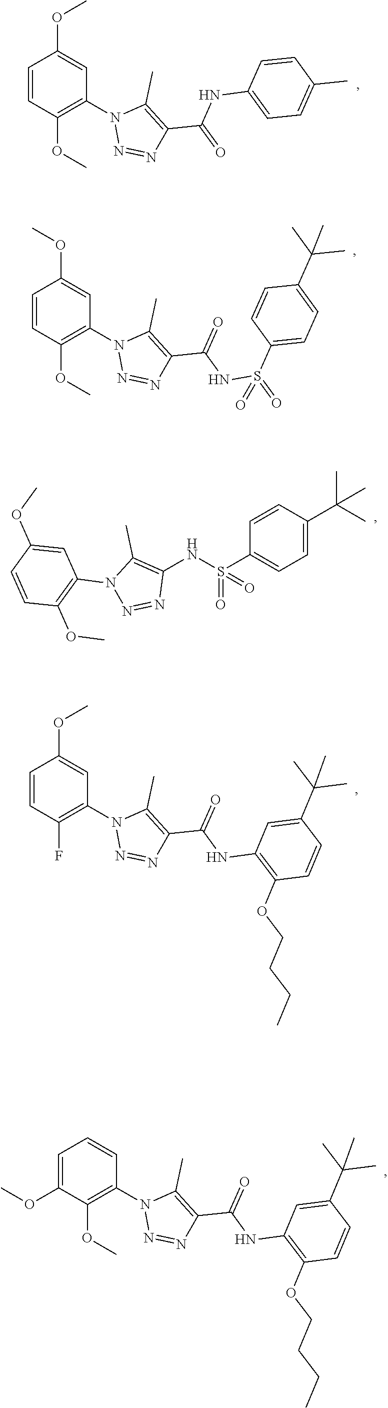

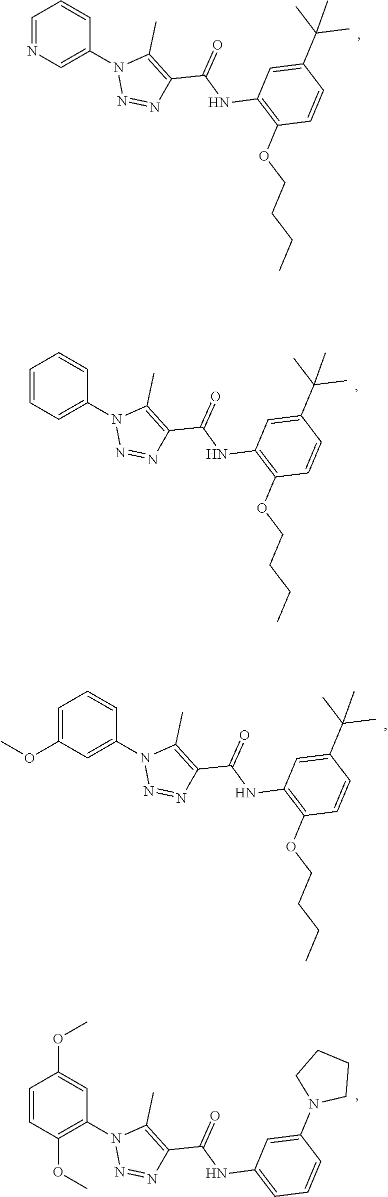

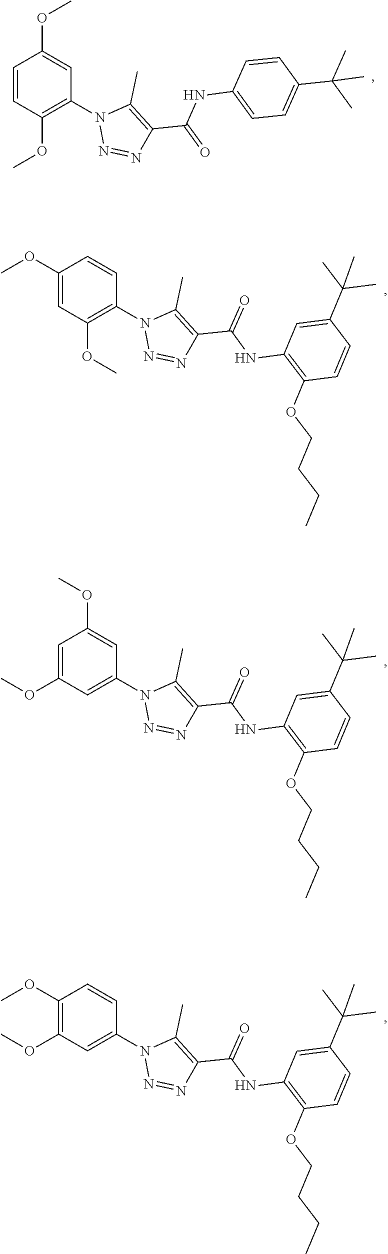

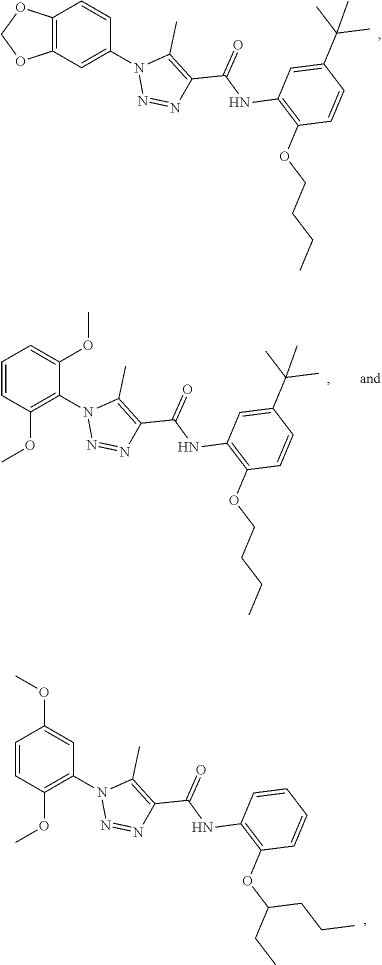 [SMILES: CCCCOc1ccc(C(C)(C)C)cc1NC(=O)c1nnn(-c2ccc3c(c2)OCO3)c1C ; CCCCOc1ccc(C(C)(C)C)cc1NC(=O)c1nnn(-c2c(OC)cccc2OC)c1C ; CCCC(CC)Oc1ccccc1NC(=O)c1nnn(-c2cc(OC)ccc2OC)c1C]

or a pharmaceutically acceptable salt thereof.

Also disclosed are methods for decreasing an adverse drug reaction in a subject in need thereof, the method comprising administering an effective amount of a compound having a structure represented by a formula:

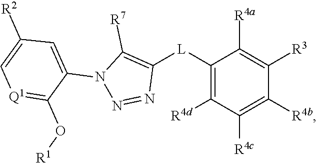

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

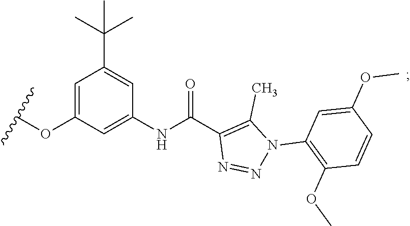

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof.

Also disclosed are methods for decreasing an adverse drug reaction in a subject in need thereof, the method comprising administering an effective amount of a compound having a structure represented by a formula:

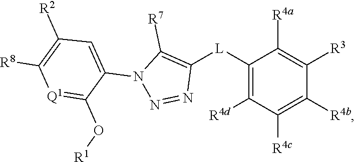

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is selected from C1-C8 alkyl, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

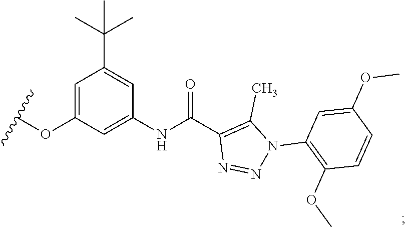

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl; wherein R⁸ is selected from hydrogen and halogen; and wherein R¹⁰ is selected from hydrogen and C1-C4 alkyl, or a pharmaceutically acceptable salt thereof.

Also disclosed are methods for decreasing an adverse drug reaction in a subject in need thereof, the method comprising an effective amount of a compound selected from:

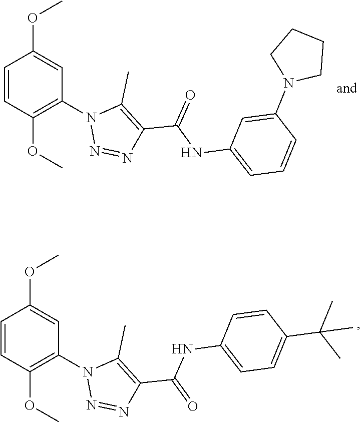 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

Also disclosed are methods of decreasing an adverse drug reaction in a subject in need thereof, the method comprising administering an effective amount of a compound selected from:

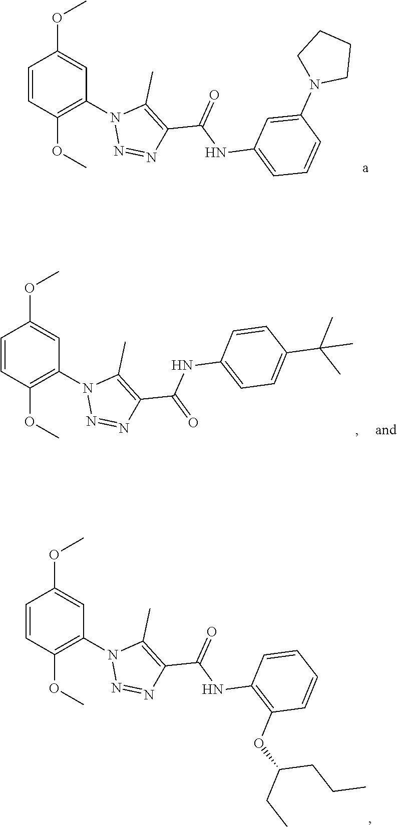 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1 ; CCC[C@H](CC)Oc1ccccc1NC(=O)c1nnn(-c2cc(OC)ccc2OC)c1C]

or a pharmaceutically acceptable salt thereof.

Also disclosed are methods for treating a disorder of uncontrolled cellular proliferation in a subject in need thereof, the method comprising administering to the subject an effective amount of a compound having a structure represented by a formula:

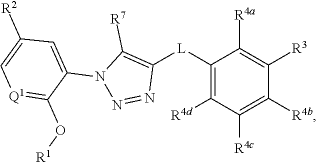

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl. C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

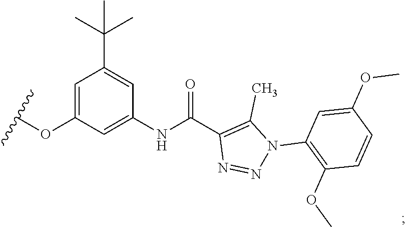

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected form hydrogen and C1-C4 alkyl, and wherein R¹⁰ is selected from hydrogen and C1-C4 alkyl, or a pharmaceutically acceptable salt thereof.

Also disclosed are methods for treating a disorder of uncontrolled cellular proliferation in a subject in need thereof, the method comprising administering to the subject an effective amount of a compound having a structure represented by a formula:

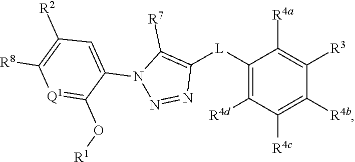

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is selected from C1-C8 alkyl, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

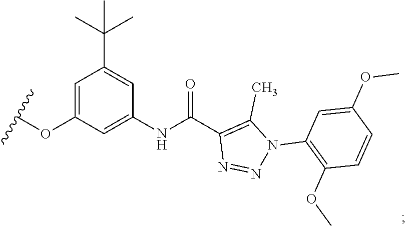

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl; wherein R⁸ is selected from hydrogen and halogen; and wherein R¹⁰ is selected from hydrogen and C1-C4 alkyl, or a pharmaceutically acceptable salt thereof.

Also disclosed are methods for treating a disorder of uncontrolled cellular proliferation in a subject in need thereof, the method comprising administering to the subject an effective amount of a compound selected from:

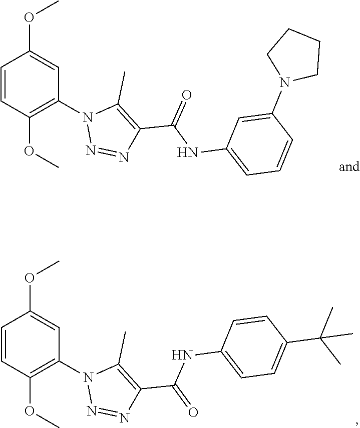 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

Also disclosed are methods for treating a disorder of uncontrolled cellular proliferation in a subject in need thereof, the method comprising administering to the subject an effective amount of a compound selected from:

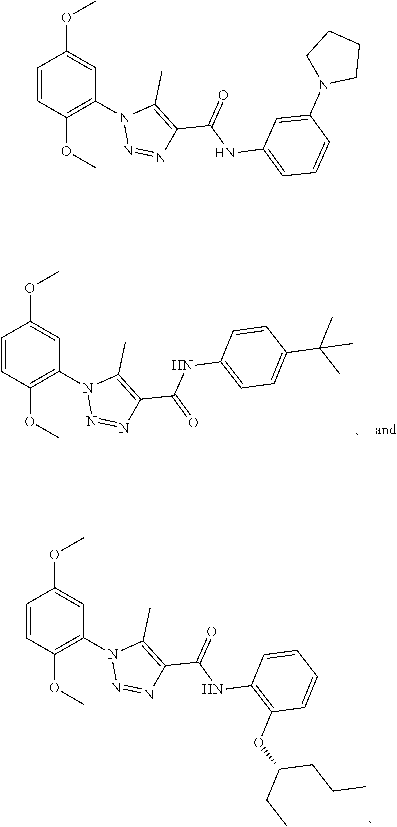 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1 ; CCC[C@H](CC)Oc1ccccc1NC(=O)c1nnn(-c2cc(OC)ccc2OC)c1C]

or a pharmaceutically acceptable salt thereof.

Also disclosed are kits comprising a compound having a structure represented by a formula:

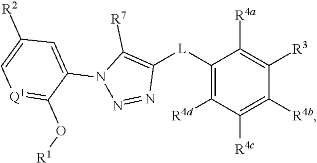

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)OR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

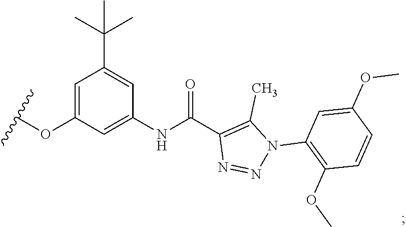

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof, and one or more selected from: an agent known to increase pregnane X receptor (PXR) activity; an agent known for the treatment of a disorder associated with pregnane X receptor (PXR) dysfunction; administering the compound in connection with a disorder associated with pregnane X receptor (PXR) dysfunction; instructions for decreasing an adverse drug reaction; and instructions for treating a disorder associated with pregnane X receptor (PXR) dysfunction.

Also disclosed are kits comprising a compound having a structure represented by a formula:

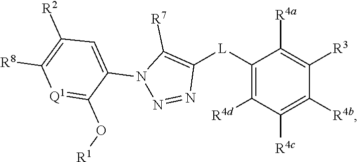

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is selected from C1-C8 alkyl, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, Ct-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂), OR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

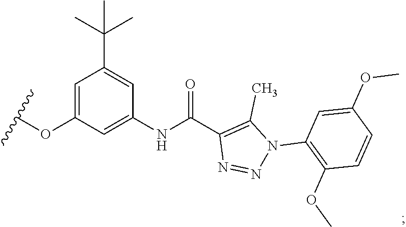

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl; wherein R⁸ is selected from hydrogen and halogen; and wherein R¹⁰ is selected from hydrogen and C1-C4 alkyl, or a pharmaceutically acceptable salt thereof, and one or more selected from: (a) an agent known to increase pregnane X receptor (PXR) activity; (b) an agent known for the treatment of a disorder associated with pregnane X receptor (PXR) dysfunction; (c) administering the compound in connection with a disorder associated with pregnane X receptor (PXR) dysfunction; (d) instructions for decreasing an adverse drug reaction; and (e) instructions for treating a disorder associated with pregnane X receptor (PXR) dysfunction.

Also disclosed are kits comprising a compound selected from:

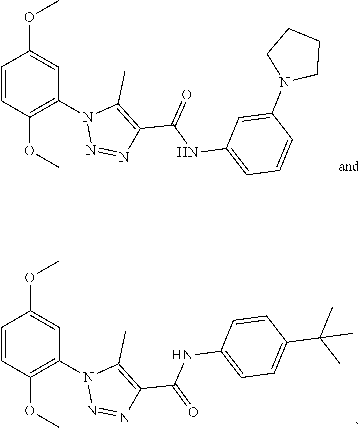 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof, and one or more selected from: an agent known to increase pregnane X receptor (PXR) activity; an agent known for the treatment of a disorder associated with pregnane X receptor (PXR) dysfunction; administering the compound in connection with a disorder associated with pregnane X receptor (PXR) dysfunction; instructions for decreasing an adverse drug reaction; and instructions for treating a disorder associated with pregnane X receptor (PXR) dysfunction.

Also disclosed are kits comprising a compound selected from:

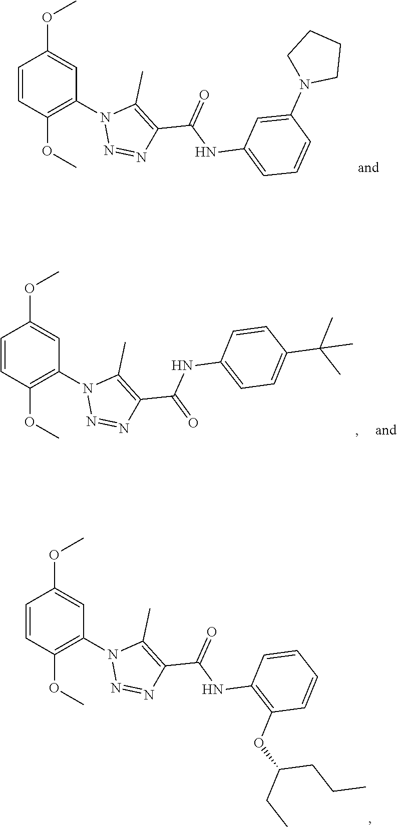 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1 ; CCC[C@H](CC)Oc1ccccc1NC(=O)c1nnn(-c2cc(OC)ccc2OC)c1C]

or a pharmaceutically acceptable salt thereof, and one or more selected from: (a) an agent known to increase pregnane X receptor (PXR) activity; (b) an agent known for the treatment of a disorder associated with pregnane X receptor (PXR) dysfunction; (c) administering the compound in connection with a disorder associated with pregnane X receptor (PXR) dysfunction; (d) instructions for decreasing an adverse drug reaction; and (e) instructions for treating a disorder associated with pregnane X receptor (PXR) dysfunction.

Also disclosed are methods of treating a disorder associated with uncontrolled cellular proliferation activity in a subject, the method comprising administering to the subject an effective amount of a compound having a structure represented by a formula:

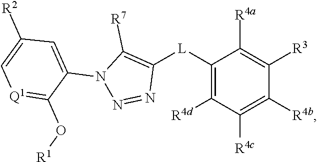

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂), OR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

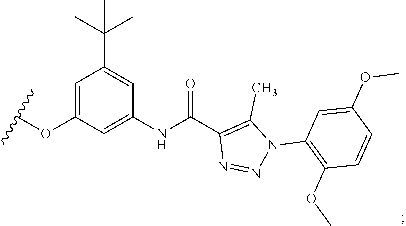

wherein R¹⁴, when present, is selected from hydrogen and C1-C4 alkyl wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof.

Also disclosed are methods for treating a bowel disorder in a subject in need thereof, the method comprising administering to the subject an effective amount of a compound having a structure represented by a formula:

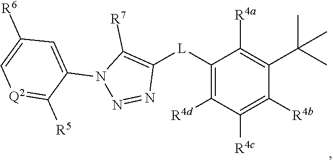

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy, and wherein R⁷ is selected hydrogen and C1-C4 alkyl, or a pharmaceutically acceptable salt thereof.

Also disclosed are methods of treating a bowel disorder in a subject in need thereof, the method comprising administering to the subject an effective amount of a compound having a structure represented by a formula:

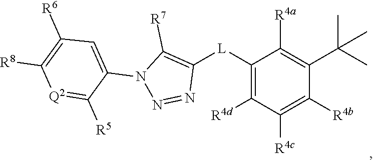

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy; wherein R⁷ is selected from hydrogen and C1-C4 alkyl; and wherein R⁸ is selected from hydrogen and C1-C4 alkoxy, or a pharmaceutically acceptable salt thereof.

Also disclosed are methods for treating a bowel disorder in a subject in need thereof, the method comprising administering to the subject an effective amount of a compound selected from:

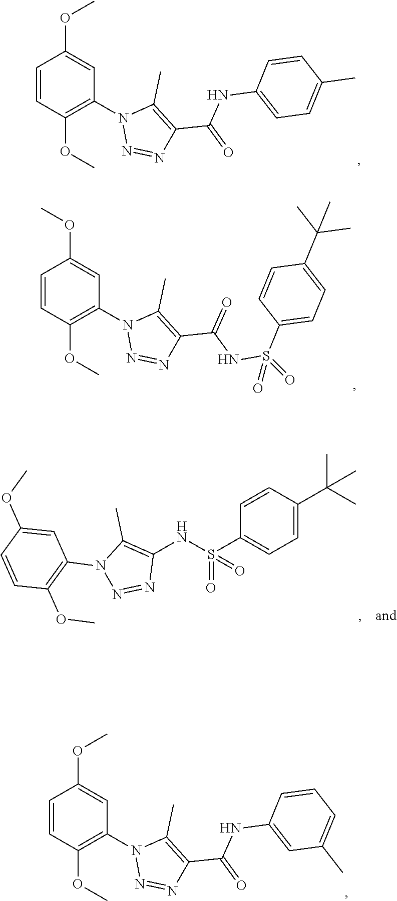 [SMILES: COc1ccc(OC)c(-n2nnc(C(=O)Nc3ccc(C)cc3)c2C)c1 ; COc1ccc(OC)c(-n2nnc(C(=O)NS(=O)(=O)c3ccc(C(C)(C)C)cc3)c2C)c1 ; COc1ccc(OC)c(-n2nnc(NS(=O)(=O)c3ccc(C(C)(C)C)cc3)c2C)c1 ; COc1ccc(OC)c(-n2nnc(C(=O)Nc3cccc(C)c3)c2C)c1]

or a pharmaceutically acceptable salt thereof.

Also disclosed are kits comprising a compound having a structure represented by a formula:

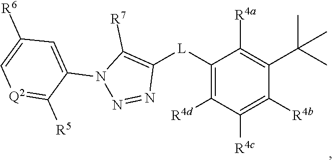

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q² is selected from N and CR¹²; wherein R¹², when present, is selected from hydrogen and C1-C4 alkoxy; wherein each of R^{4a}, R^{4b}, R^{4c}, and R⁴⁶ is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy, and wherein R⁷ is selected from hydrogen and C1-C4 alkyl, or a pharmaceutically acceptable salt thereof, and one or more selected from: an agent known for the treatment of a disorder associated with pregnane X receptor (PXR) dysfunction; instructions for administering the compound in connection with a disorder associated with pregnane X receptor (PXR) dysfunction; instructions for decreasing an adverse drug reaction; and instructions for treating a disorder associated with pregnane X receptor (PXR) dysfunction.

Also disclosed are kits comprising a compound having a structure represented by a formula:

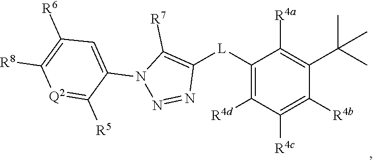

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q² is selected from N and CR¹²; wherein R¹², when present, is selected from hydrogen and C1-C4 alkoxy; wherein each of R^{4a}, R^{4b}, R^{4c}, and R⁴⁶ is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy; and wherein R⁷ is selected from hydrogen and C1-C4 alkyl; and wherein R⁸ is selected from hydrogen and C1-C4 alkoxy, or a pharmaceutically acceptable salt thereof, and one or more selected from: (a) an agent known for the treatment of a disorder associated with pregnane X receptor (PXR) dysfunction; (b) instructions for administering the compound in connection with a disorder associated with pregnane X receptor (PXR) dysfunction; (c) instructions for decreasing an adverse drug reaction; and (d) instructions for treating a disorder associated with pregnane X receptor (PXR) dysfunction.

Also disclosed are kits comprising a compound selected from:

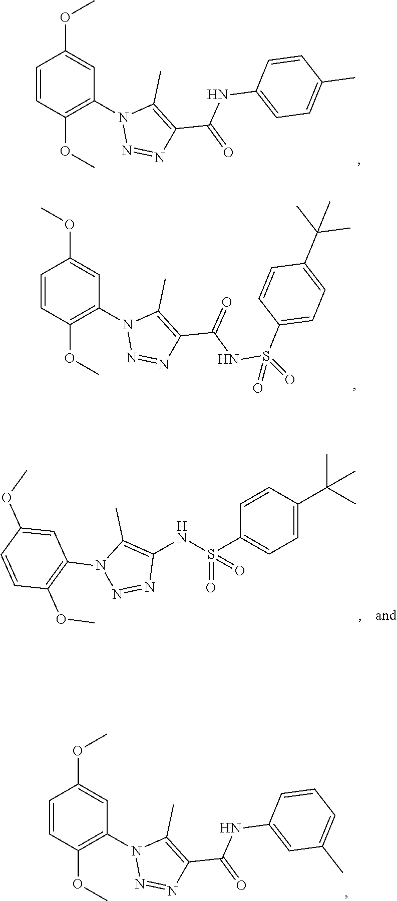 [SMILES: COc1ccc(OC)c(-n2nnc(C(=O)Nc3ccc(C)cc3)c2C)c1 ; COc1ccc(OC)c(-n2nnc(C(=O)NS(=O)(=O)c3ccc(C(C)(C)C)cc3)c2C)c1 ; COc1ccc(OC)c(-n2nnc(NS(=O)(=O)c3ccc(C(C)(C)C)cc3)c2C)c1 ; COc1ccc(OC)c(-n2nnc(C(=O)Nc3cccc(C)c3)c2C)c1]

or a pharmaceutically acceptable salt thereof, and one or more selected from: an agent known for the treatment of a disorder associated with pregnane X receptor (PXR) dysfunction; instructions for administering the compound in connection with a disorder associated with pregnane X receptor (PXR) dysfunction; instructions for decreasing an adverse drug reaction; and instructions for treating a disorder associated with pregnane X receptor (PXR) dysfunction.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1A-D show representative spectral data for compound no. B34. Specifically, a ¹H NMR spectrum (FIG. 1A), a ¹³C NMR spectrum (FIG. 1B), a HRMS spectrum (FIG. 1C), and HPLC traces (FIG. 1D) are shown.

FIG. 2A-D show representative spectral data for compound no. M1. Specifically, a ¹H NMR spectrum (FIG. 2A), a ¹³C NMR spectrum (FIG. 2B), a HRMS spectrum (FIG. 2C), and HPLC traces (FIG. 2D) are shown.

FIG. 3A-D show representative spectral data for compound no. M2. Specifically, a ¹H NMR spectrum (FIG. 3A), a ¹³C NMR spectrum (FIG. 3B), a HRMS spectrum (FIG. 3C), and HPLC traces (FIG. 3D) are shown.

FIG. 4A and FIG. 4B show representative supercritical fluid chromatography (SFC) spectra of compound nos. M1 (FIG. 4A, bottom) and M2 (FIG. 4A, top) and B34 (FIG. 4B).

FIG. 5A-D show representative spectral data for compound no. M9. Specifically, a ¹H NMR spectrum (FIG. 5A), a ¹³C NMR spectrum (FIG. 5B), a HRMS spectrum (FIG. 5C), and HPLC traces (FIG. 5D) are shown.

FIG. 6A-D show representative spectral data for compound no. M10. Specifically, a ¹H NMR spectrum (FIG. 6A), a ¹³C NMR spectrum (FIG. 6B), a HRMS spectrum (FIG. 6C), and HPLC traces (FIG. 6D) are shown.

FIG. 7A-D show representative spectral data for compound no. M11. Specifically, a ¹H NMR spectrum (FIG. 7A), a ¹³C NMR spectrum (FIG. 7B), a HRMS spectrum (FIG. 7C), and HPLC traces (FIG. 7D) are shown.

FIG. 8A-D show representative spectral data for compound no. M12. Specifically, a ¹H NMR spectrum (FIG. 8A), a ¹³C NMR spectrum (FIG. 8B), a HRMS spectrum (FIG. 8C), and HPLC traces (FIG. 8D) are shown.

FIG. 9A-D show representative spectral data for compound no. M3. Specifically, a ¹H NMR spectrum (FIG. 9A), a ¹³C NMR spectrum (FIG. 9B), a HRMS spectrum (FIG. 9C), and HPLC traces (FIG. 9D) are shown.

FIG. 10A-D show representative spectral data for compound no. M4. Specifically, a ¹H NMR spectrum (FIG. 10A), a ¹³C NMR spectrum (FIG. 10B), a HRMS spectrum (FIG. 10C), and HPLC traces (FIG. 10D) are shown.

FIG. 11A-D show representative spectral data for compound no. M5. Specifically, a ¹H NMR spectrum (FIG. 11A), a ¹³C NMR spectrum (FIG. 11B), a HRMS spectrum (FIG. 11C), and HPLC traces (FIG. 11D) are shown.

FIG. 12A-D show representative spectral data for compound no. M6. Specifically, a ¹H NMR spectrum (FIG. 12A), a ¹³C NMR spectrum (FIG. 12B), a HRMS spectrum (FIG. 12C), and HPLC traces (FIG. 12D) are shown.

FIG. 13A-D show representative spectral data for compound no. M7. Specifically, a ¹H NMR spectrum (FIG. 13A), a ¹³C NMR spectrum (FIG. 13B), a HRMS spectrum (FIG. 13C), and HPLC traces (FIG. 13D) are shown.

FIG. 14A-D show representative spectral data for compound no. M8. Specifically, a ¹H NMR spectrum (FIG. 14A), a ¹³C NMR spectrum (FIG. 14B), a HRMS spectrum (FIG. 14C), and HPLC traces (FIG. 14D) are shown.

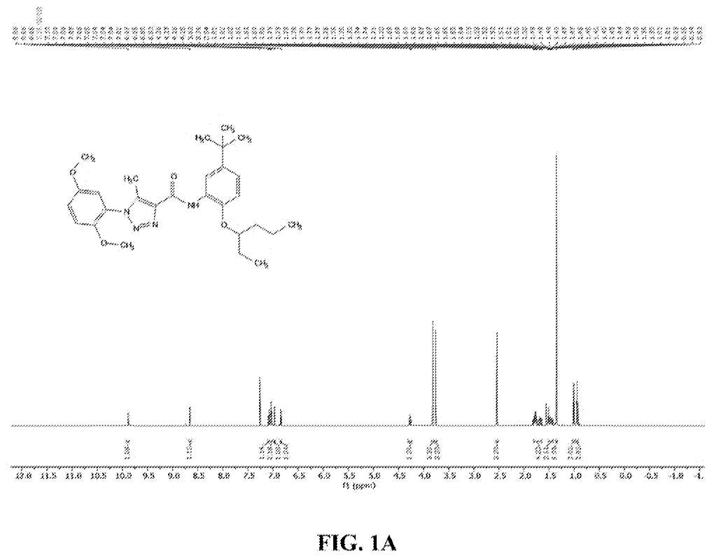

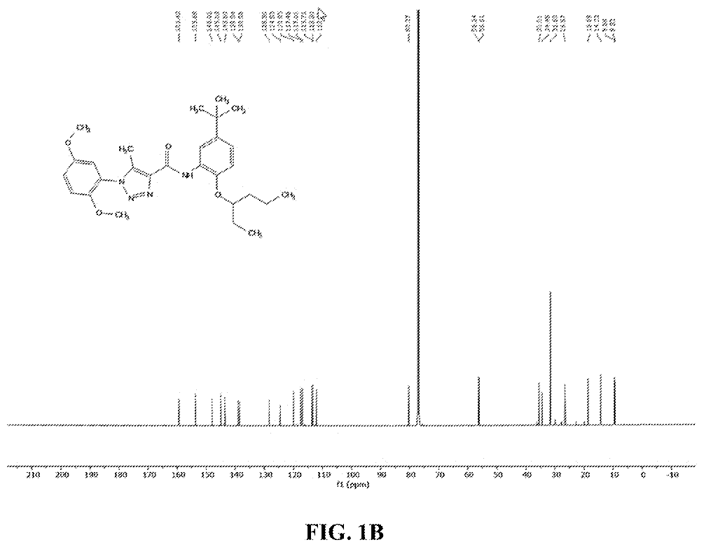

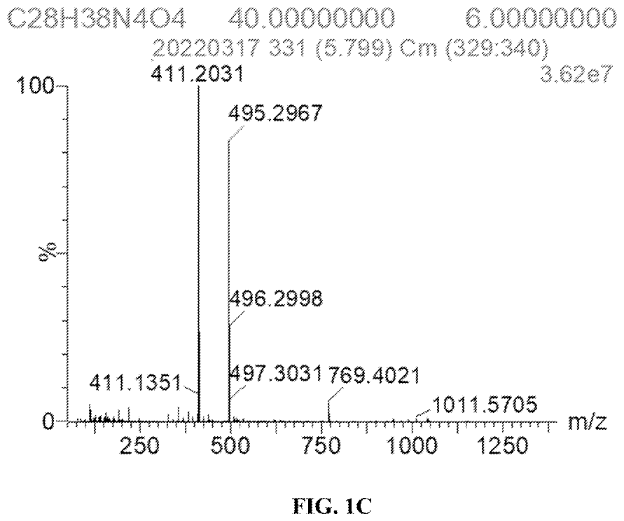

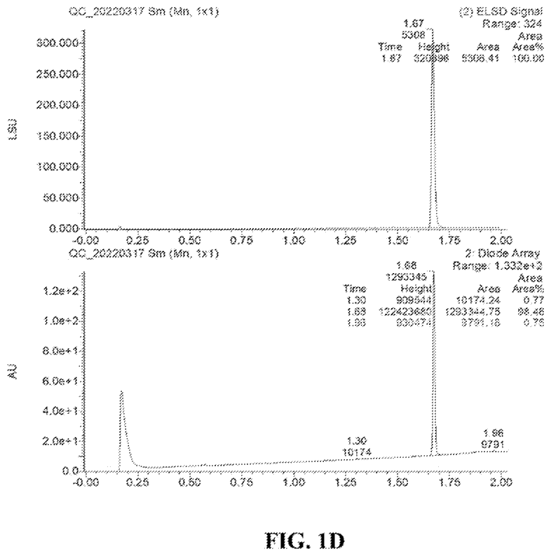

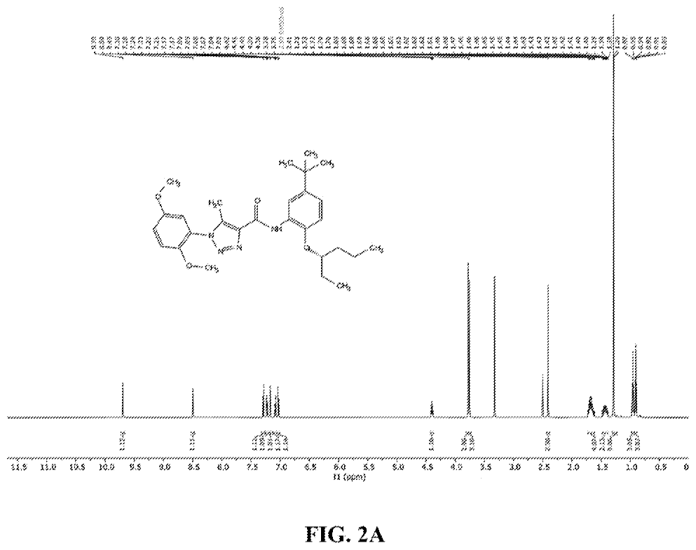

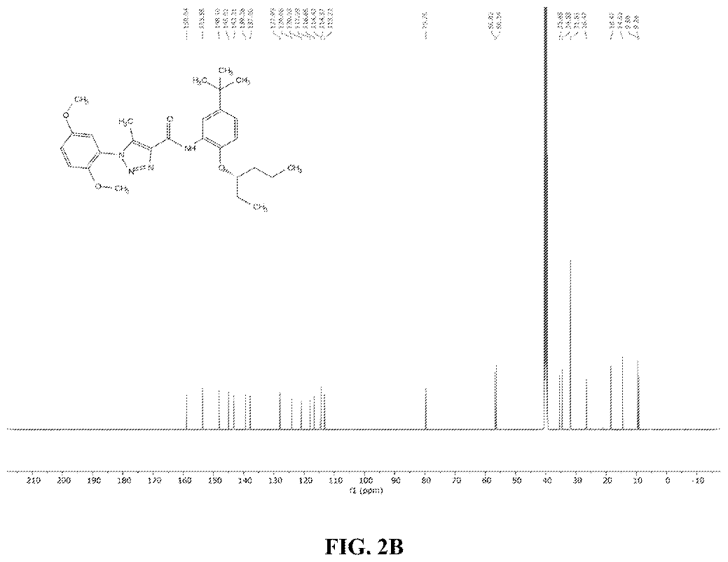

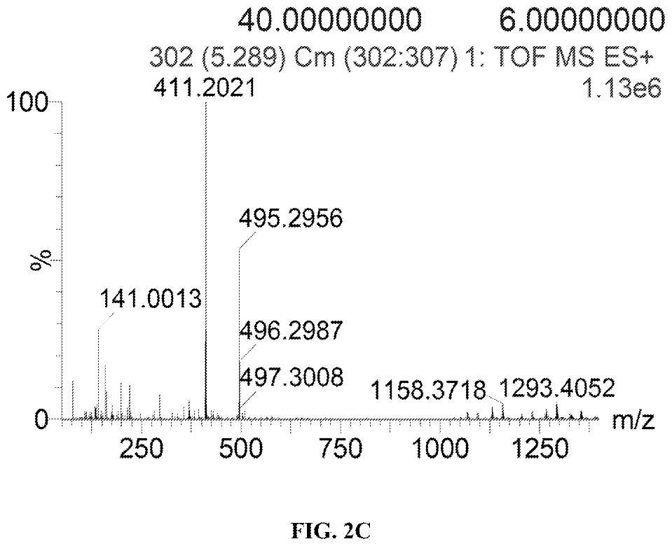

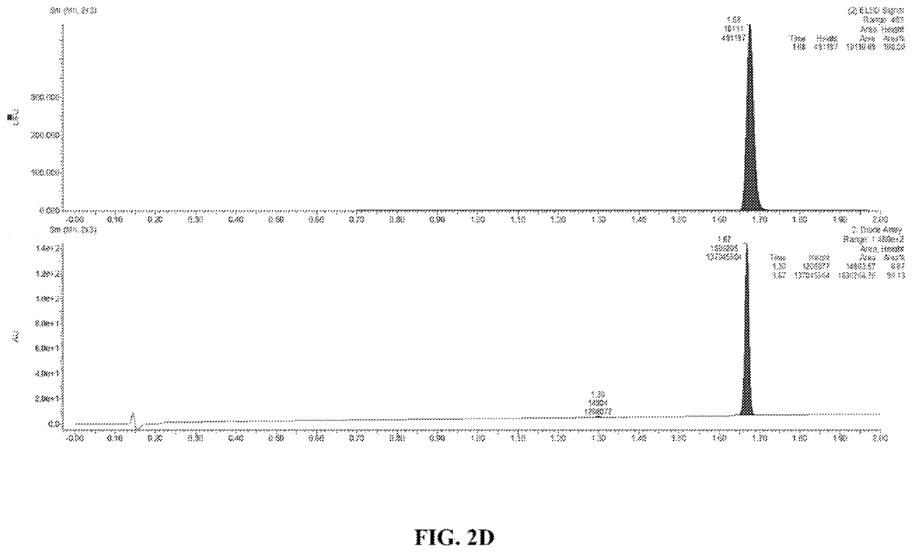

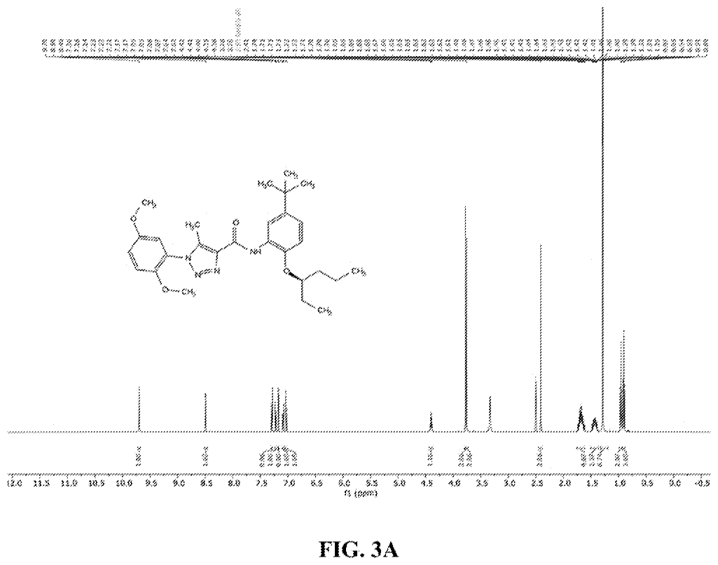

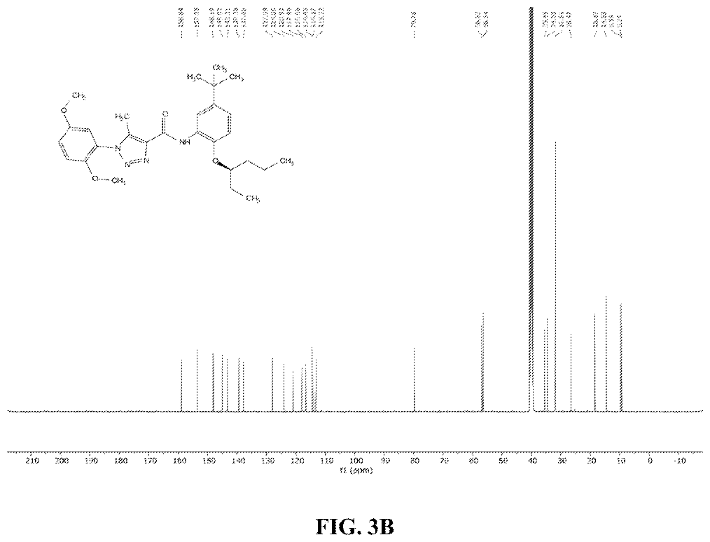

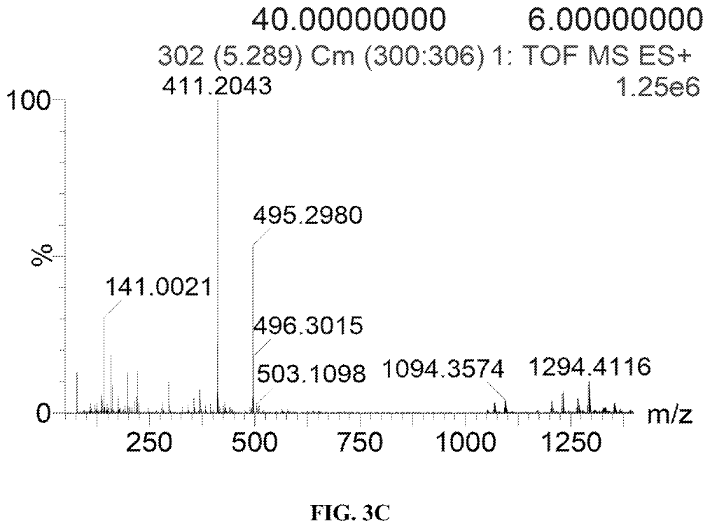

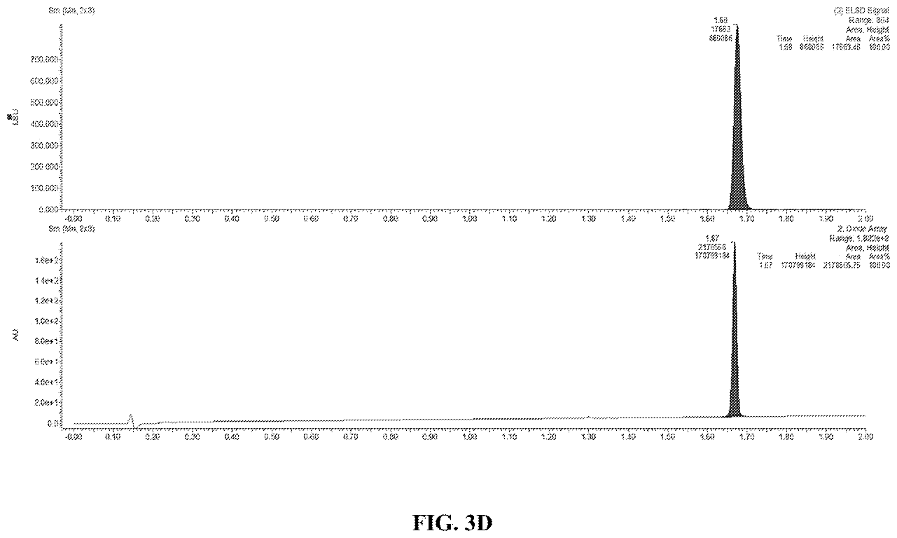

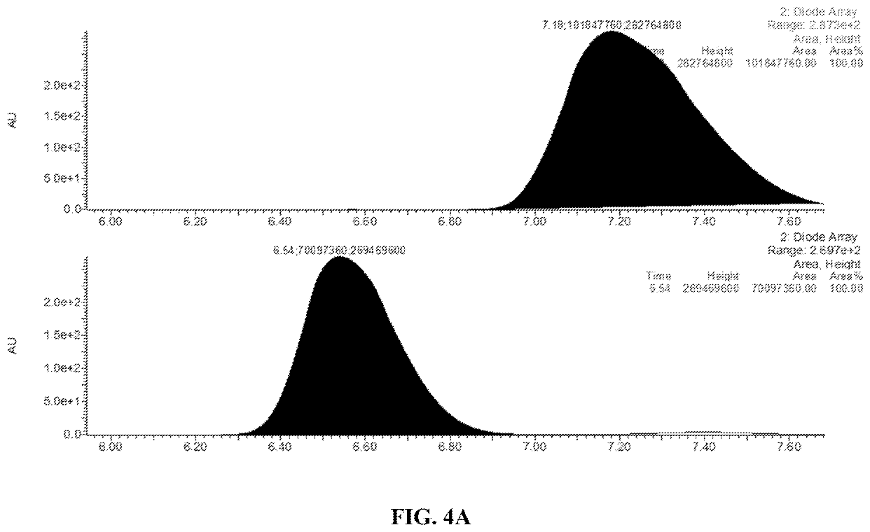

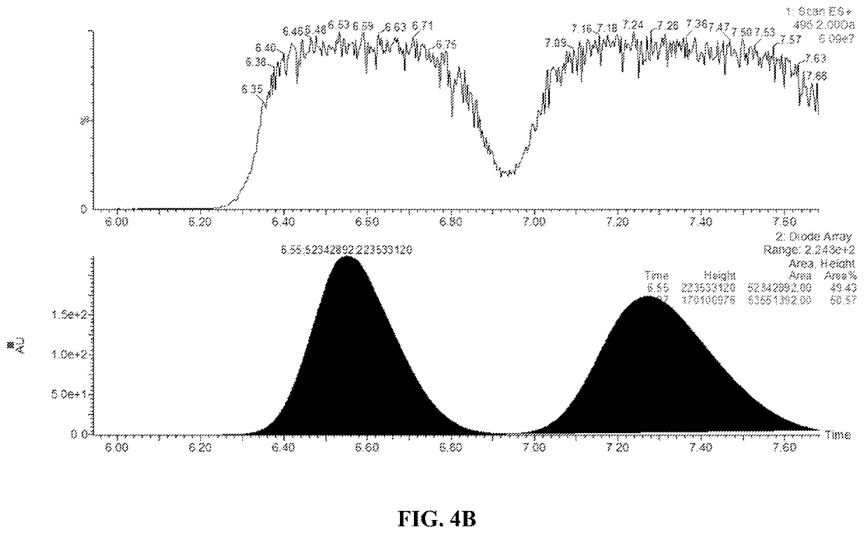

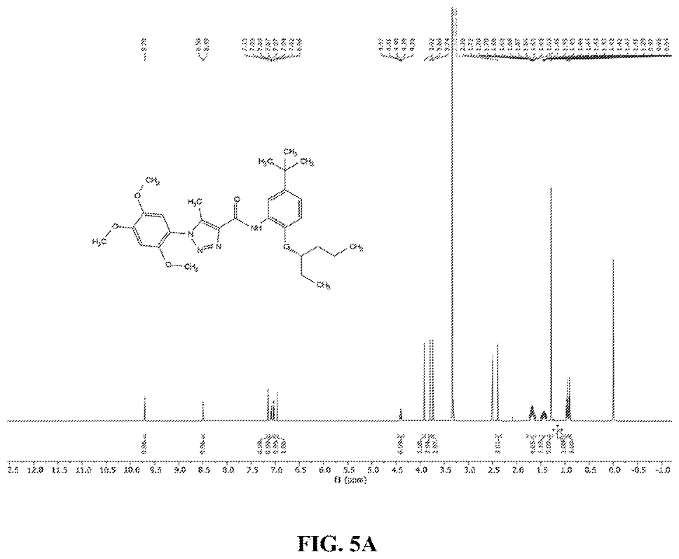

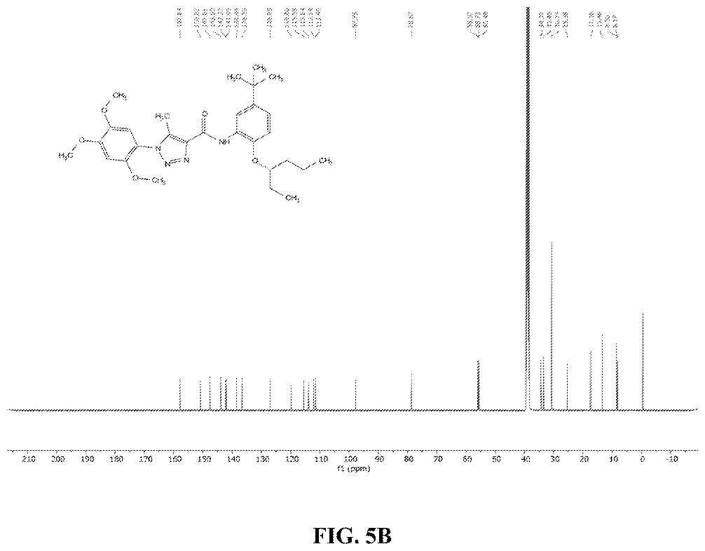

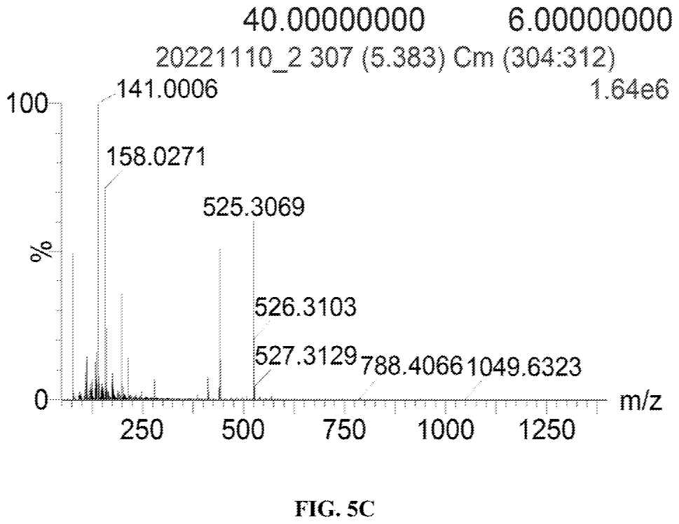

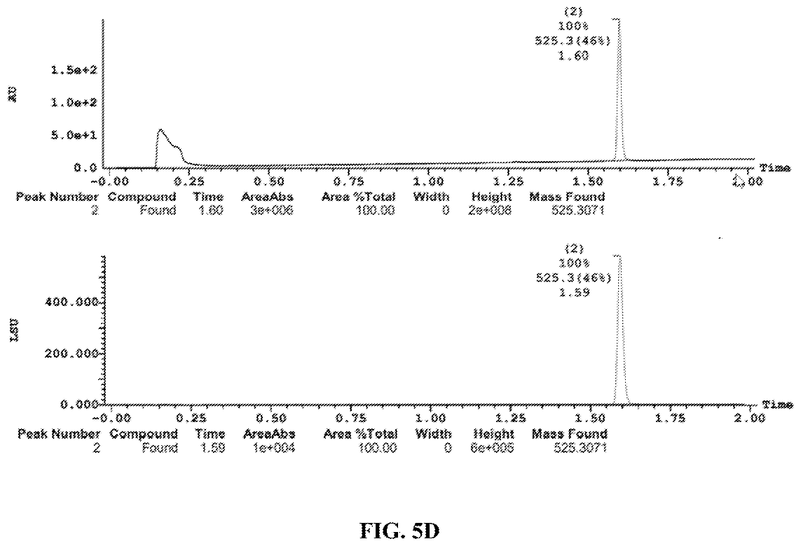

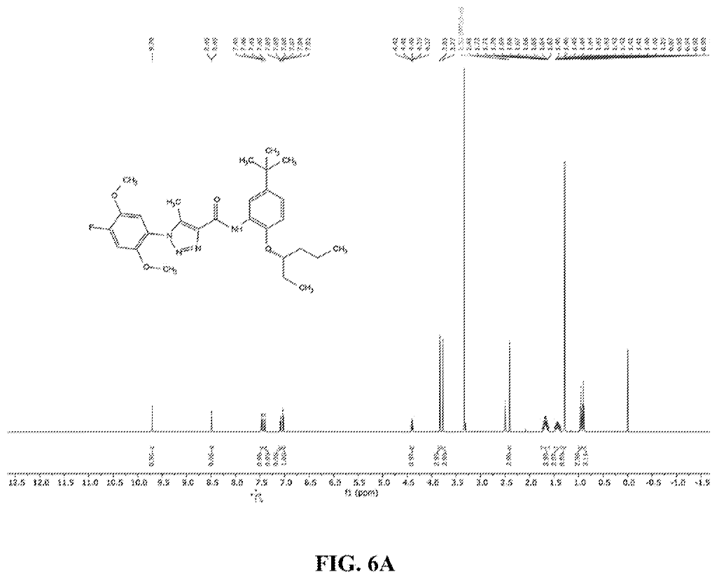

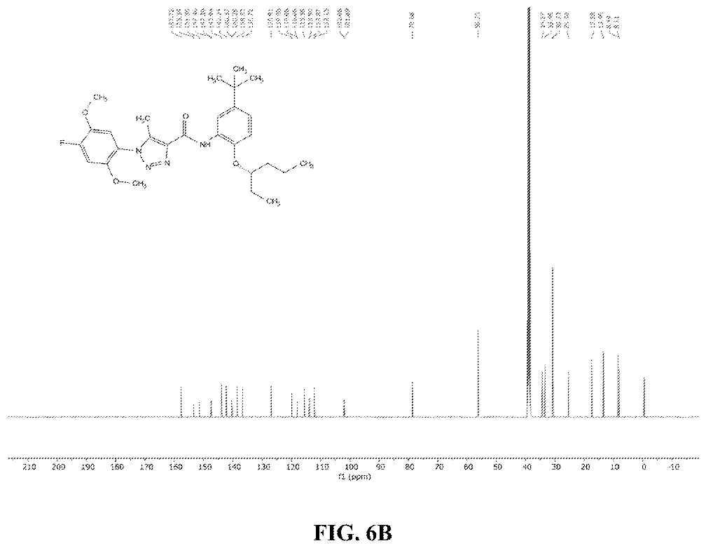

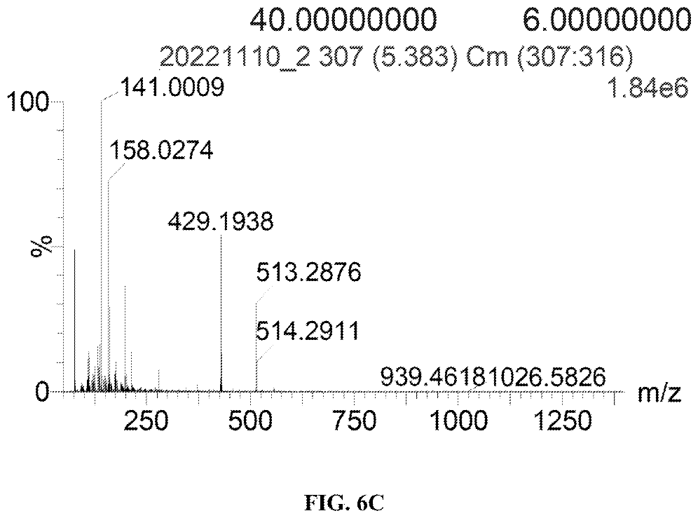

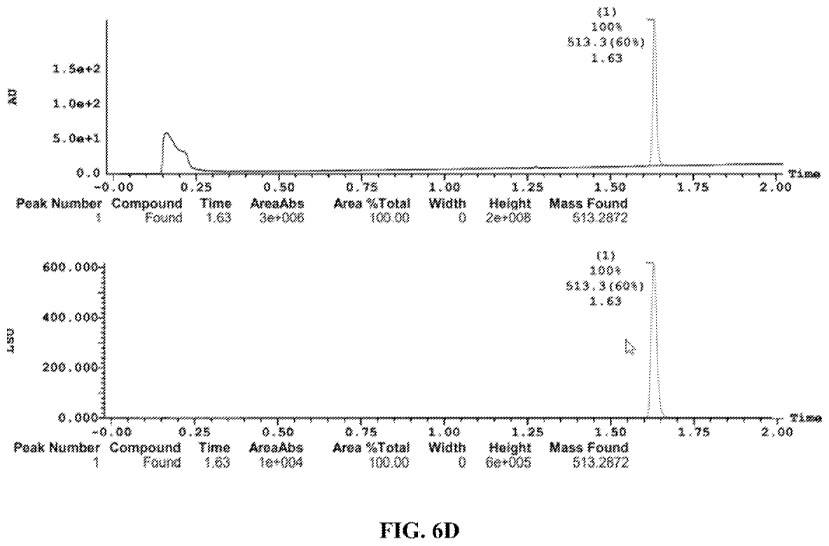

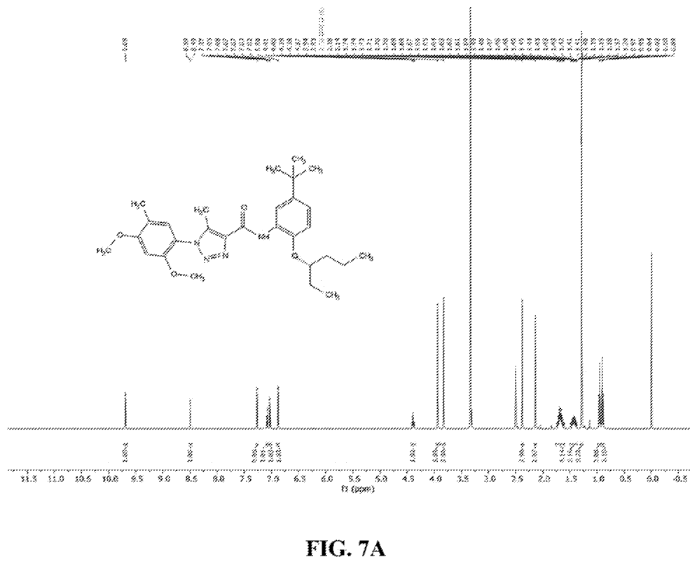

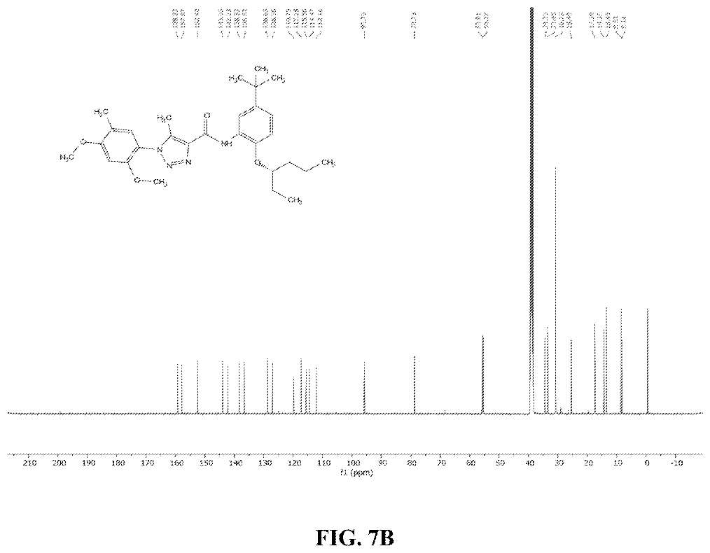

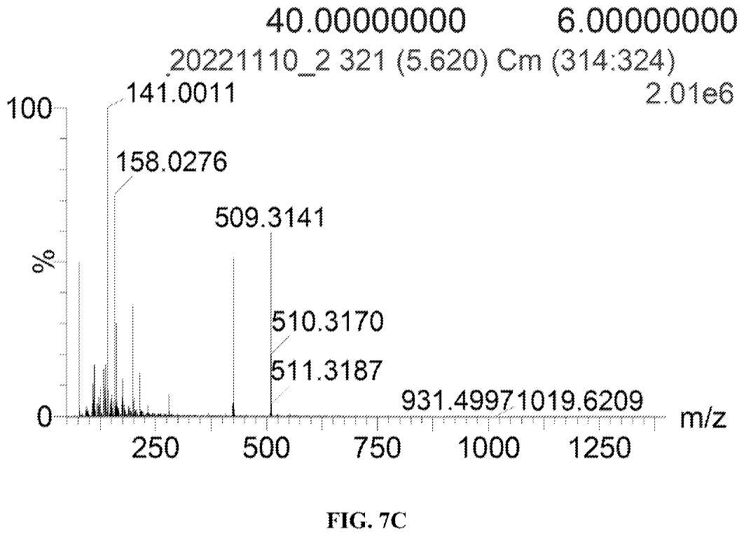

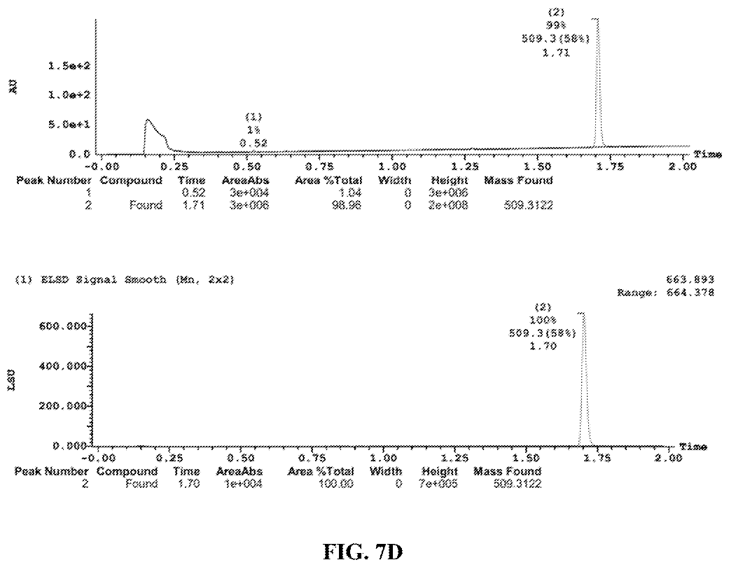

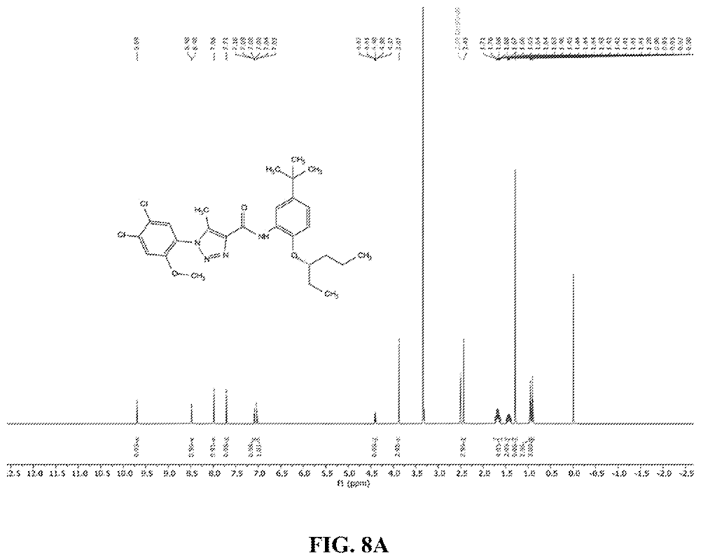

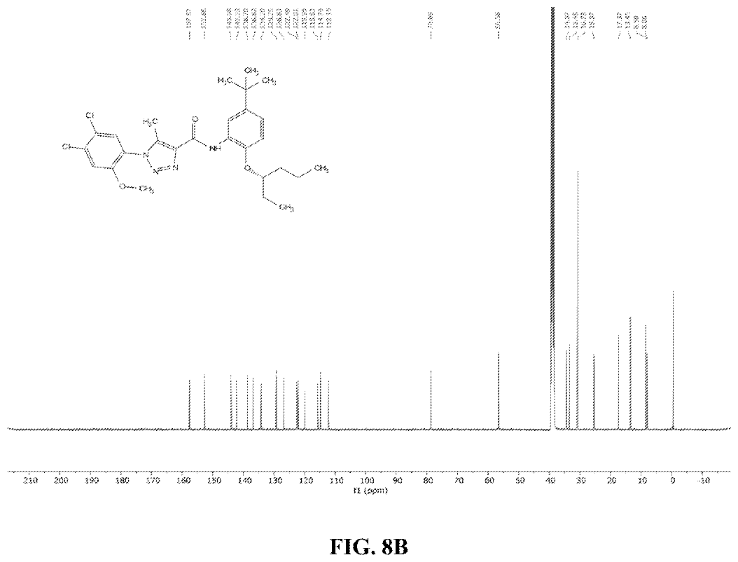

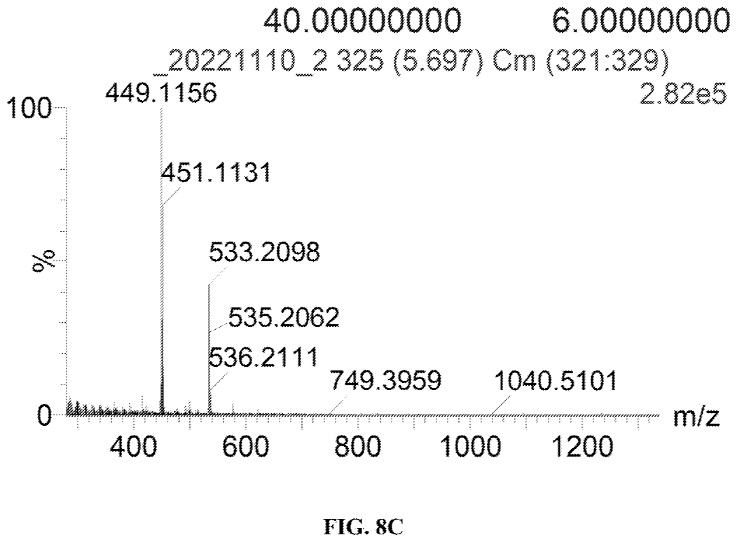

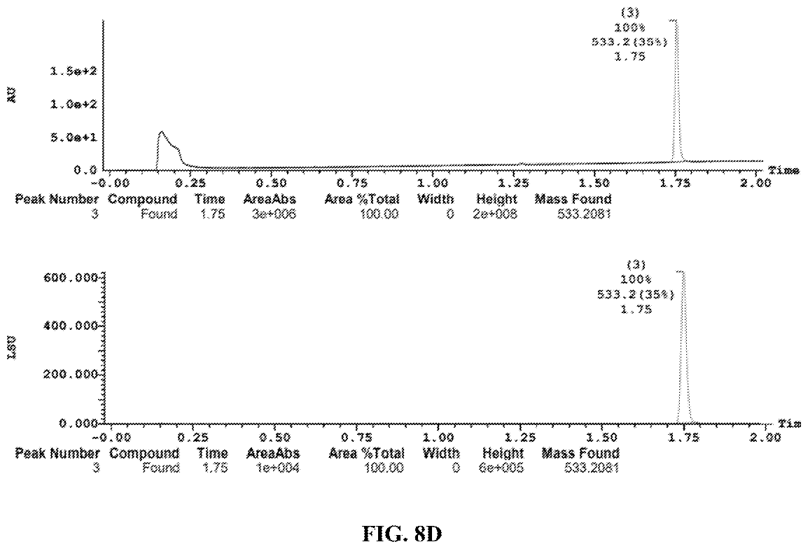

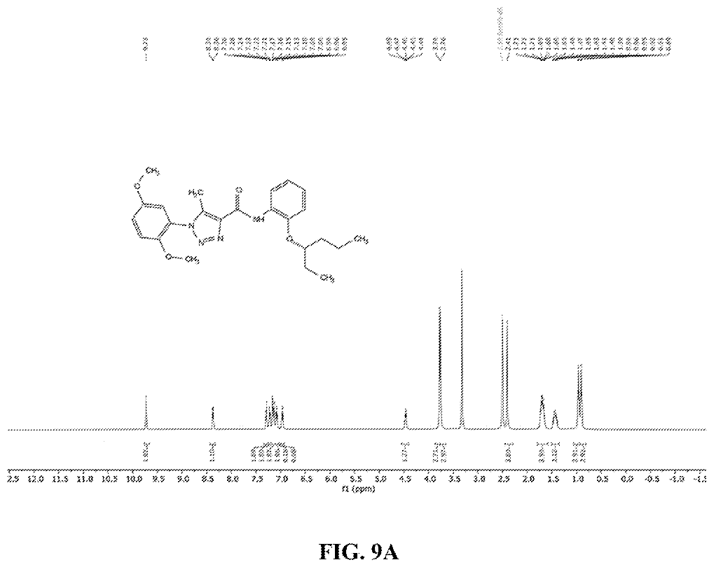

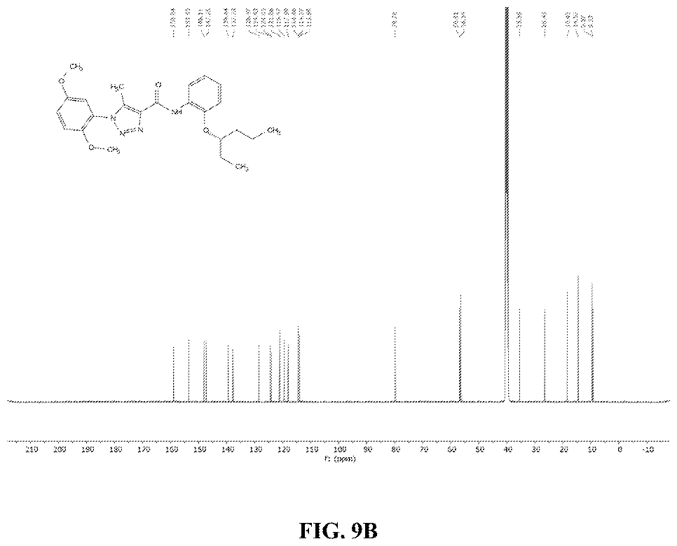

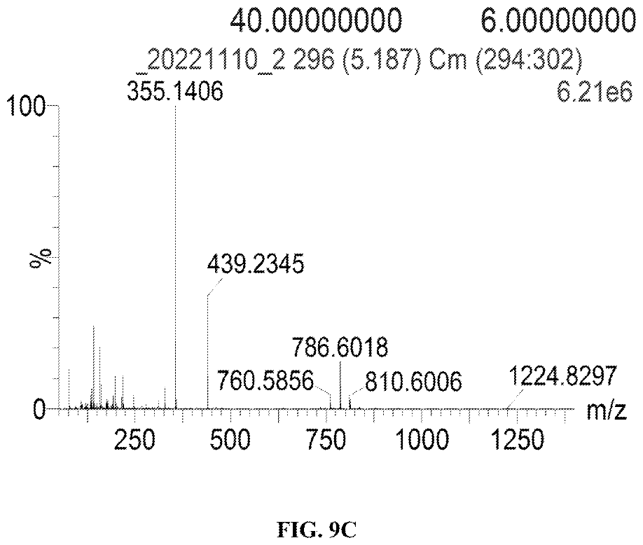

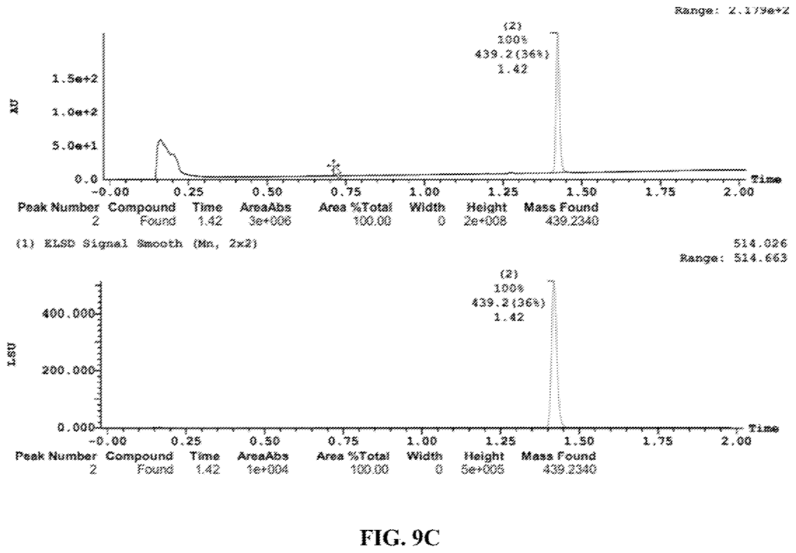

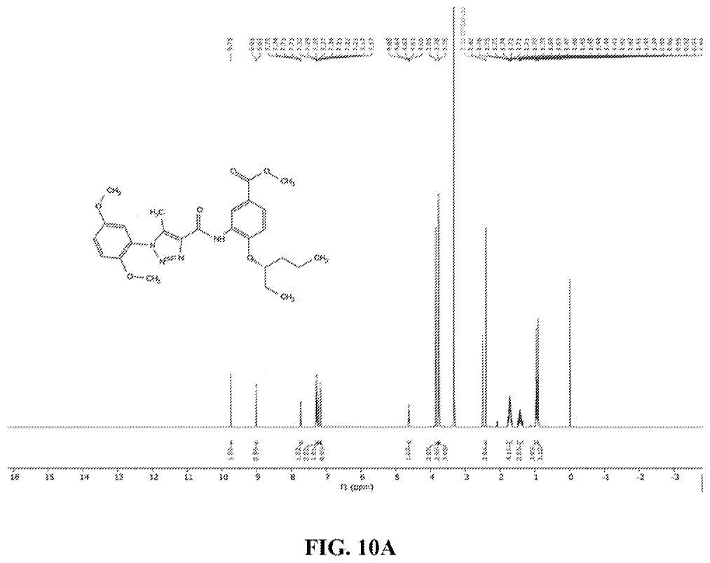

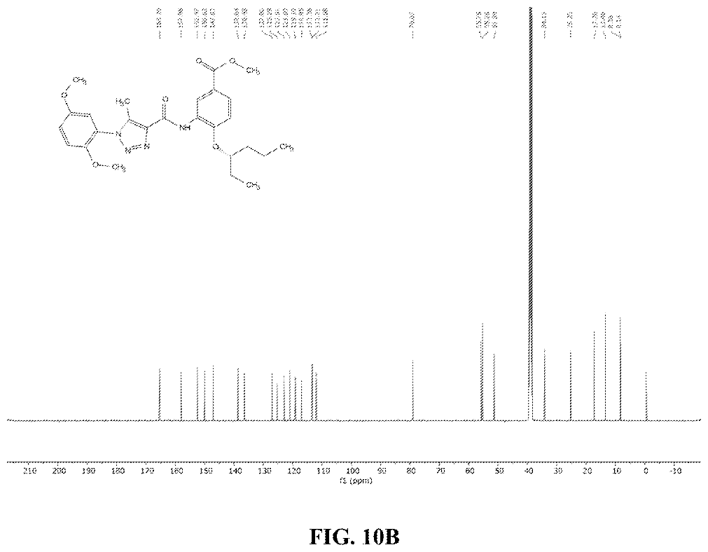

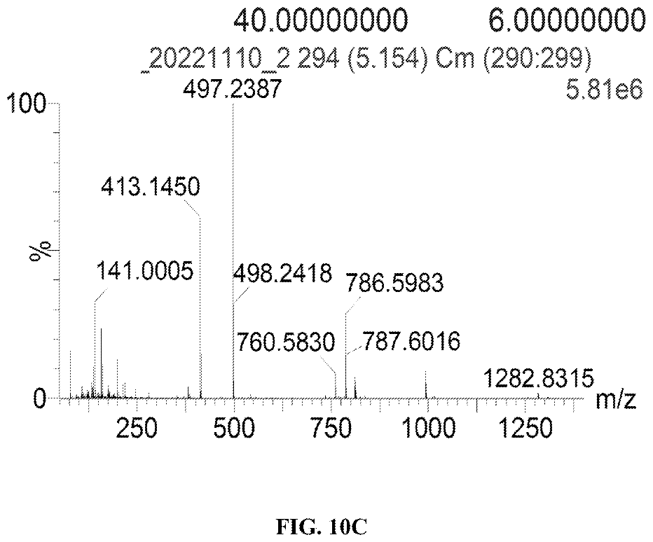

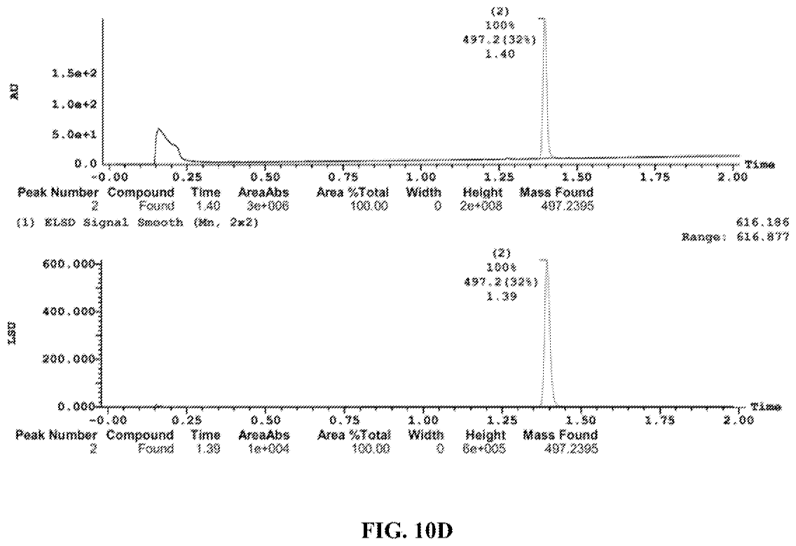

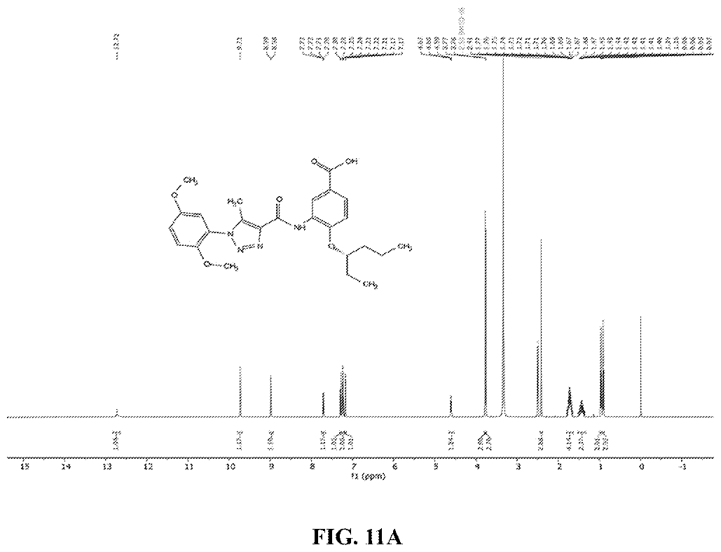

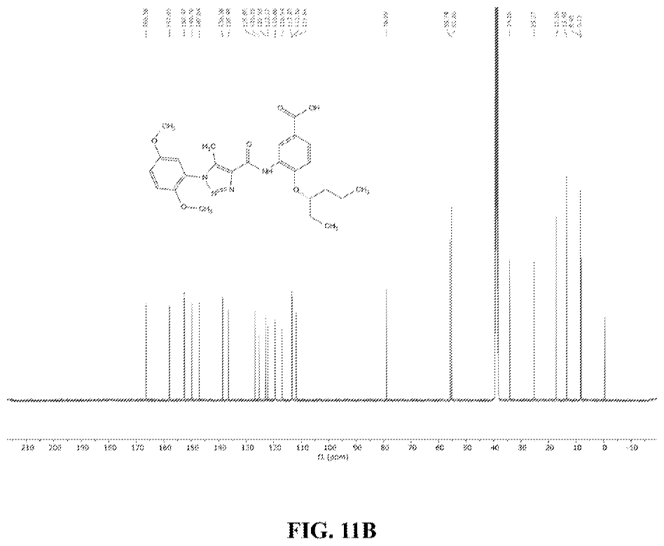

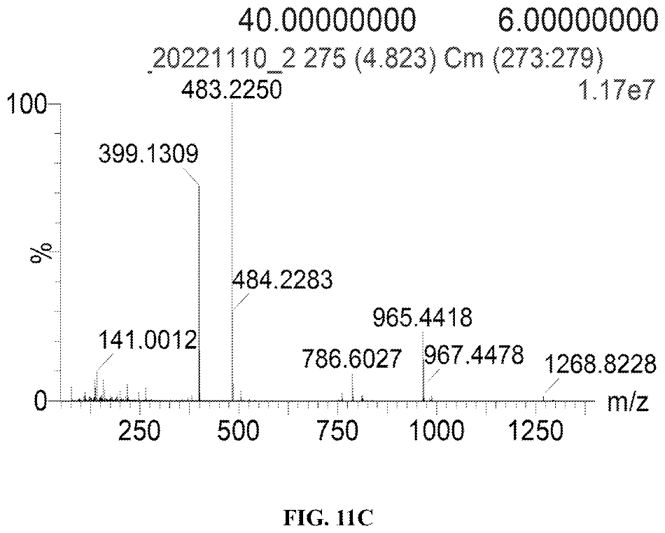

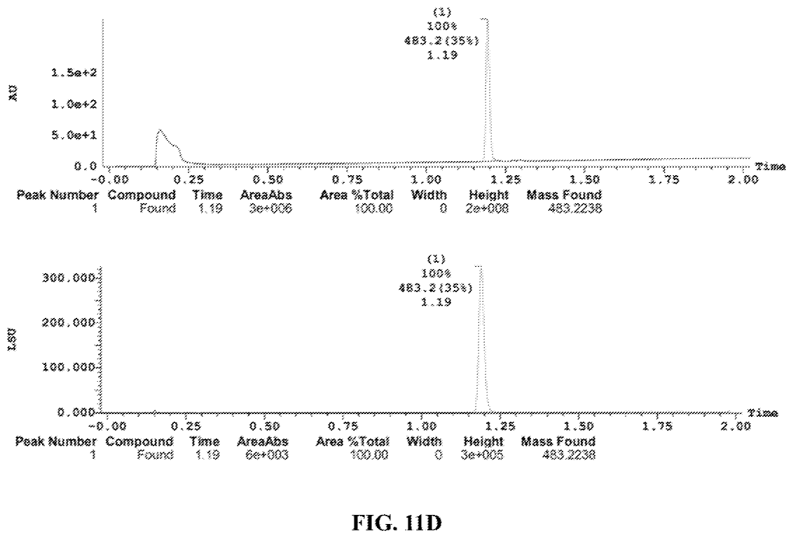

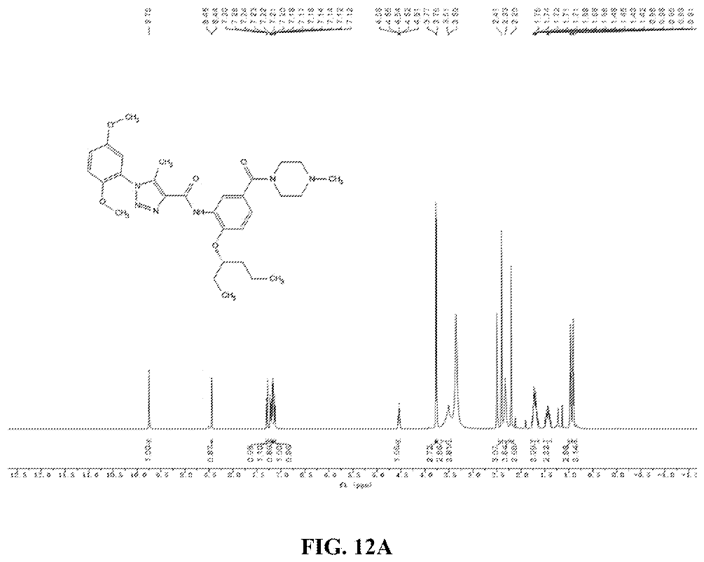

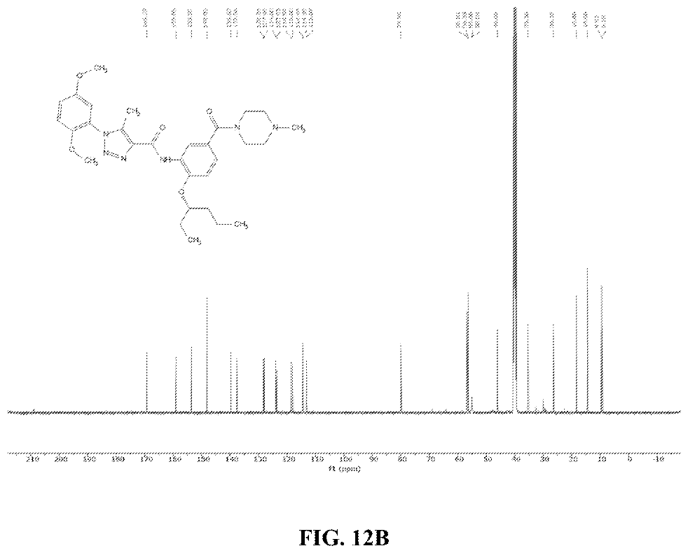

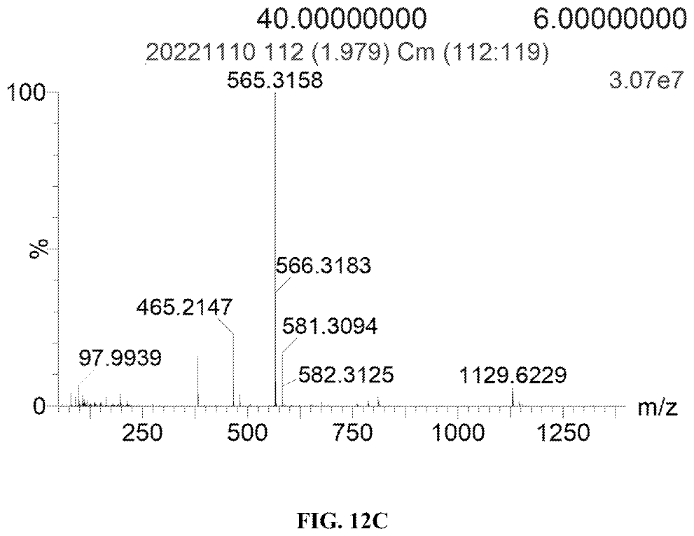

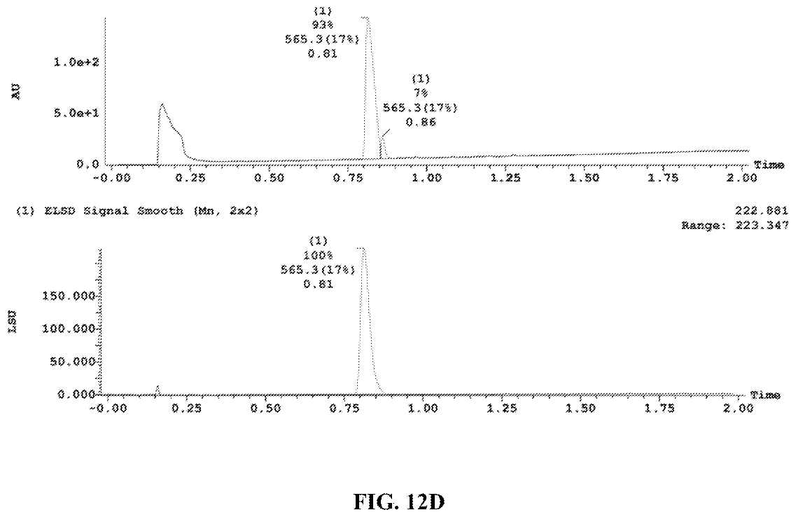

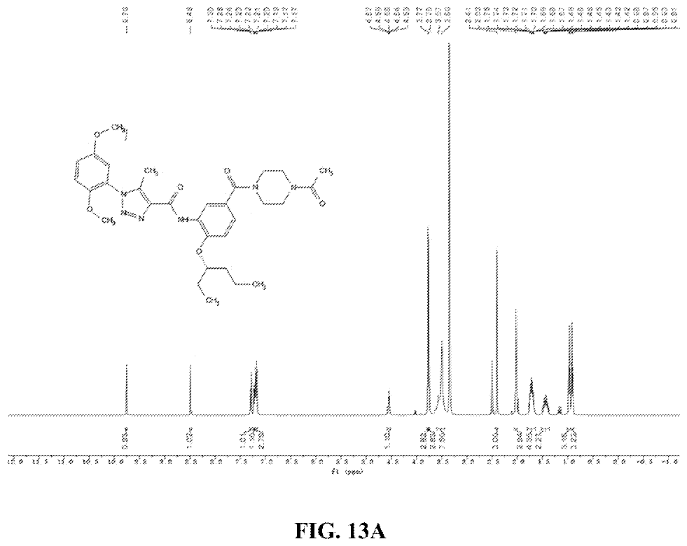

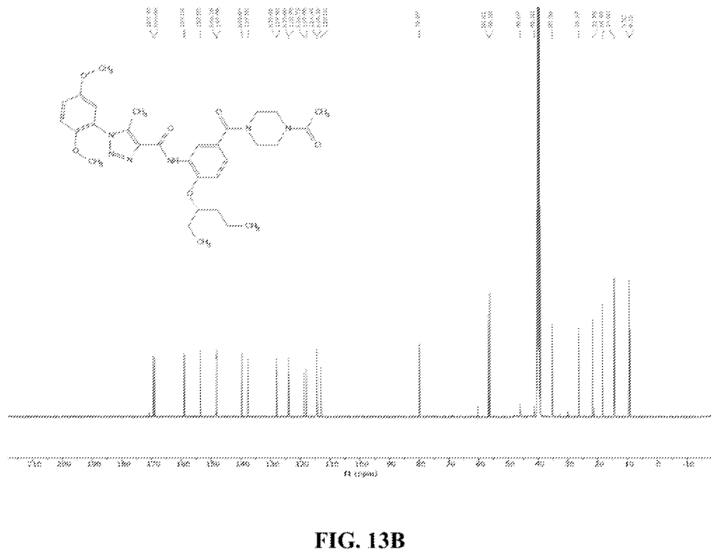

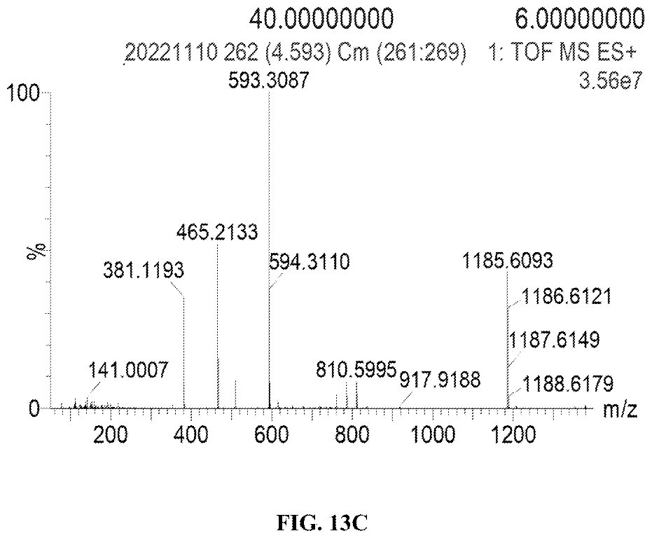

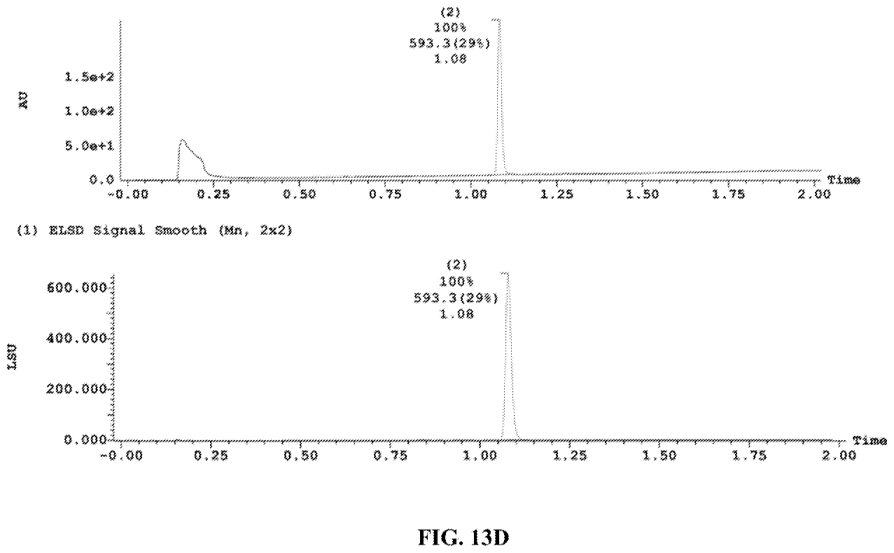

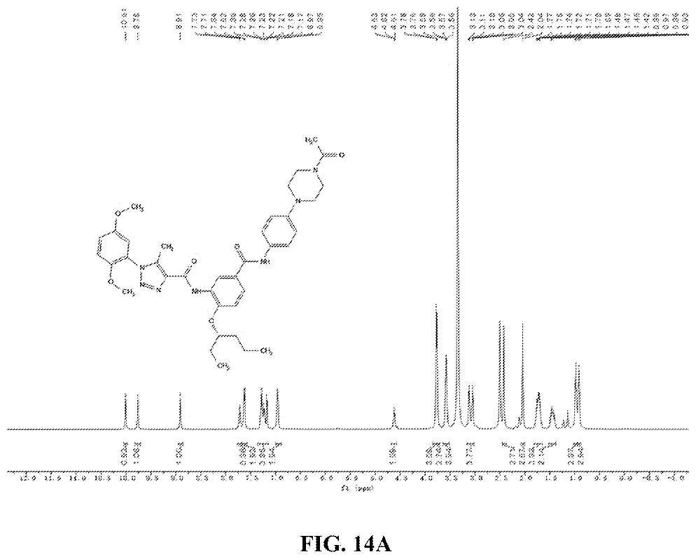

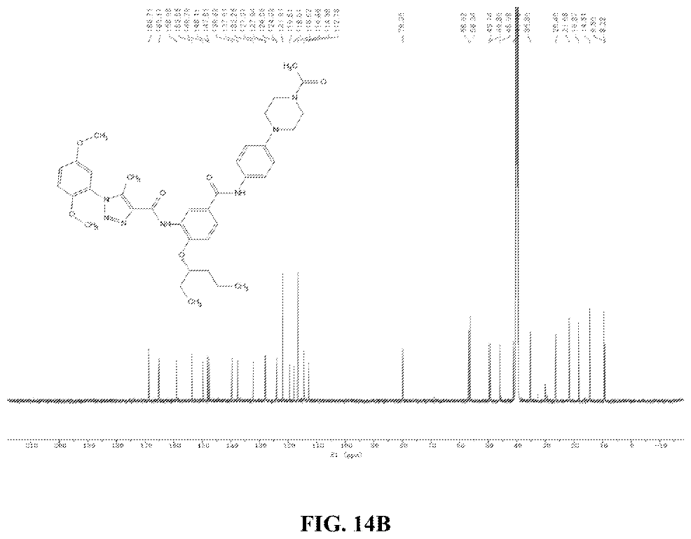

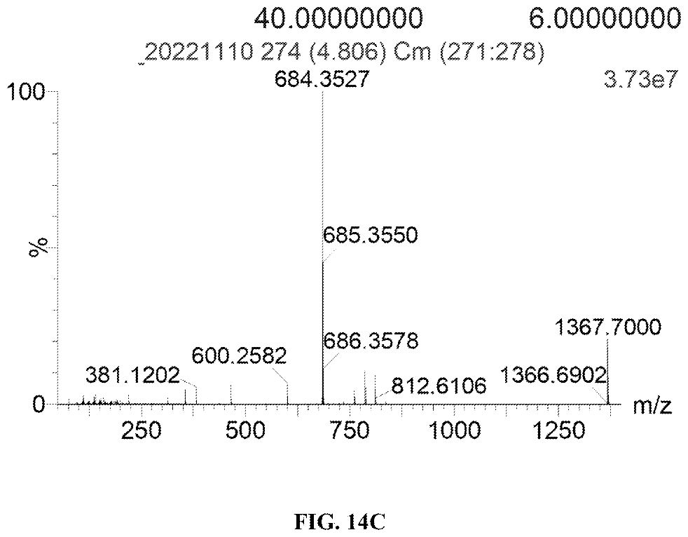

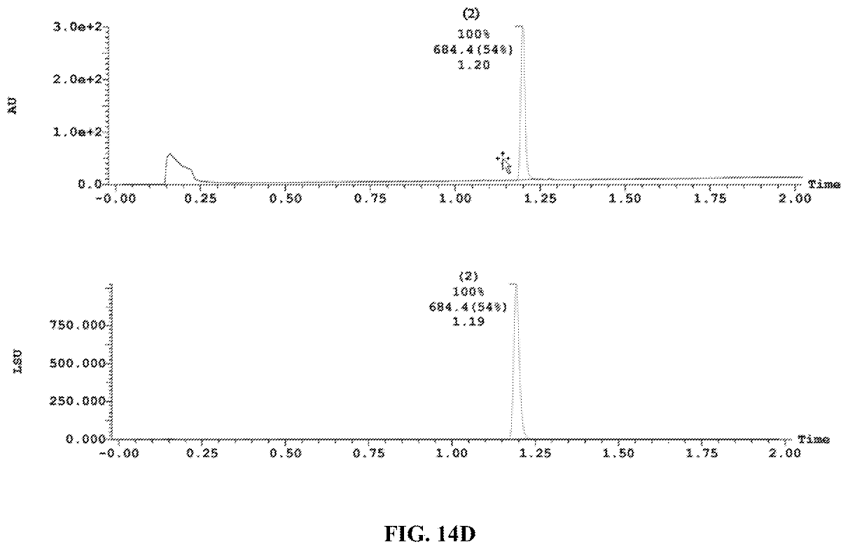

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the terms “PXR” and “pregnane X receptor” can be used interchangeably and refer to a nuclear receptor protein encoded by the NR1I2 gene, which is a transcriptional regulator of cytochrome P450 genes such as CYP3A4 and CYP3A5. PXR has a human gene map locus given as 3q12-q13.3, 3q13.3, and 3q12-q13.3 by Entrez Gene, Ensembl, and HGNC, respectively. The corresponding rat and mouse genes are given the gene symbol Nrli2, and the respective gene map loci are 11q21 and 16 B3. The gene and protein have variously been referred to in the scientific literature as ONR1, BXR, SXR, PAR2, Orphan nuclear receptor PART, pregnane-activated receptor, steroid and xenobiotic receptor, MGC108643, pregnane X receptor (nuclear receptor sub family 1, group I, member 2), nuclear receptor subfamily 1 group I member 2, NR1I2, orphan nuclear receptor PXR, PXR.1, PXR.2, mPXR, and nuclear receptor subfamily 1, group 1, member 2. It can be appreciated that these terms can also be used to refer to PXR. The term PXR is understood to be inclusive of related homologous proteins in other species. The human form can be specifically designated by the term “hPXR.” The PXR protein is characterized by a DNA binding domain and a ligand binding domain (also referred to by the term “LBD”). The PXR protein forms a heterodimer with the 9-cis retinoic acid receptor RXR, and the formation of the heterodimer is required for transcriptional activation of target genes, and the heterodimer binds to the response element of the CYP3A4 or CYP3A5 promoter. The heterodimer is also believed to bind to the response elements of the ABCB1/MDR1 gene.

The major human, rat, and mouse PXR protein isoforms encoded by the PXR gene (NR1I2) are, respectively, 434, 431, and 431 amino acids. However, several major splice variants have been described at least for human encoding different isoforms, some of which have been described as using non-AUG translation initiation codons. For example, a significant human isoform is the “long isoform”, that is 473 amino acids comprising 39 additional amino acids added to the N-terminus of the major human isoform which is 434 amino acids. The LBD of the major human isoform is from amino acids 141-434, whereas the LBD of the long isoform is from amino acids 180-473.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, the subject is a mammal. A patient refers to a subject afflicted with a disease or disorder. The term “patient” includes human and veterinary subjects.

In some aspects of the disclosed methods, the subject has been diagnosed with a need for treatment of an infectious disease prior to the administering step. In some aspects of the disclosed methods, the subject has been diagnosed with a need for modulating PXR activity prior to the administering step. In some aspects of the disclosed methods, the subject has been diagnosed with having a gram positive or gram negative infection prior to the administering step. In some aspects of the disclosed methods, the subject has been identified with an infectious disease that is treatable by antagonizing the activity of PXR prior to the administering step. In some aspects of the disclosed methods, the subject has been identified with a gram positive bacterial infection prior to the administering step. In various aspects of the disclosed methods, the subject has been identified with a gram negative bacterial infection prior to the administering step. In an aspect, a subject can be treated prophylactically with a compound or composition disclosed herein, as discussed herein elsewhere.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

For example, “diagnosed with an infectious disease treatable by antagonizing PXR activity” means having been subjected to a physical examination by a person of skill, for example, a physician, and found to have a condition that can be diagnosed or treated by a compound or composition that can inhibit PXR activity. As a further example, “diagnosed with a need for treatment of an infectious disease” refers to having been subjected to a physical examination by a person of skill, for example, a physician, and found to have a condition characterized by infection with a pathogenic microbe, such as a gram positive or gram negative bacteria.

As used herein, the phrase “identified to be in need of treatment for an infectious disease,” or the like, refers to selection of a subject based upon need for treatment of the infectious disease. For example, a subject can be identified as having a need for treatment of an infectious disease (e.g., an infectious disease related to infection with a pathogenic gram negative or gram positive bacteria) based upon an earlier diagnosis by a person of skill and thereafter subjected to treatment for the infectious disease. It is contemplated that the identification can, In an aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, a target protein (e.g. the PXR protein), or other biological entity together in such a manner that the compound can affect the activity of the target, either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “EC₅₀,” is intended to refer to the concentration of a substance (e.g., a compound or a drug) that is required for 50% activation or enhancement of a biological process, or component of a process. For example, EC₅₀ can refer to the concentration of a compound that provokes a response halfway between the baseline and maximum response in an appropriate assay of the target activity. For example, an EC₅₀ for the PXR can be determined in an in vitro assay system. Such in vitro assay systems include assay such as the assays as described herein.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In an aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance. For example, an EC₅₀ for the PXR can be determined in an in vitro assay system. Such in vitro assay systems include assay such as the assays as described herein.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as -OA¹ where A¹ is alkyl or cycloalkyl as defined above. “Alkoxy” also includes polymers of alkoxy groups as just described; that is, an alkoxy can be a polyether such as -OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond.

Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the 71 clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula -NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula -SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹. —OS(O)₂A, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₄R^{o}; —(CH₂)_{0 4}OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{o}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

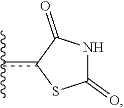

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

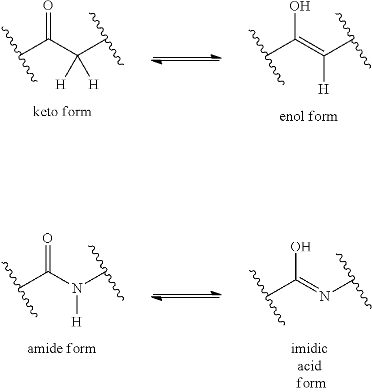 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

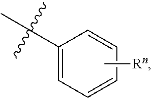

which is understood to be equivalent to a formula:

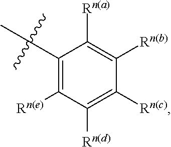

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplemental Volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C-F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In an aspect, disclosed are compounds useful as modulators of the pregnane X receptor (PXR). In a further aspect, the disclosed compounds are useful for treatment of a disorder of uncontrolled cellular proliferation, such as a cancer. In a still further aspect, the disclosed compounds are useful for decreasing an adverse drug reaction in a mammal such as, for example, an adverse drug reaction associated with administration of an anticancer agent, an antibacterial agent, a non-steroidal anti-inflammatory agent, or an anticonvulsant agent.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

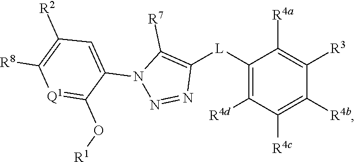

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is hydrogen, halogen, C1-C8 alkyl, C1-C8 haloalkyl, C1-C8 alkoxy, C1-C8 haloalkoxy, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂), OR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

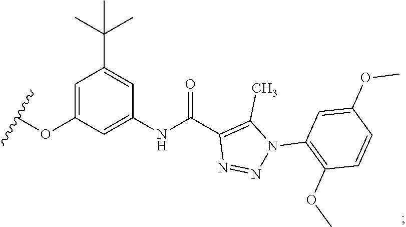

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy. C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R⁸ is selected from hydrogen, halogen, and C1-C4 alkoxy; wherein R⁷ is selected from hydrogen and C1-C4 alkyl; provided that when L is —C(O)NR¹⁰—, then R² is selected from C1-C4 haloalkyl, C1-C4 alkoxy, and C1-C4 haloalkoxy, and R³ is C1-C8 alkyl, —CO₂(C1-C4 alkyl), or —C(O)Cy², or a pharmaceutically acceptable salt thereof.

In one aspect, disclosed are compounds having a structure represented by a formula:

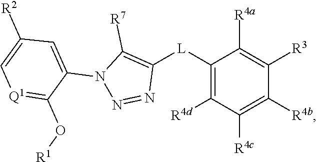

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is selected from hydrogen, halogen, C1-C8 alkyl, C1-C8 haloalkyl, C1-C8 alkoxy, and C1-C8 haloalkoxy; and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl. C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

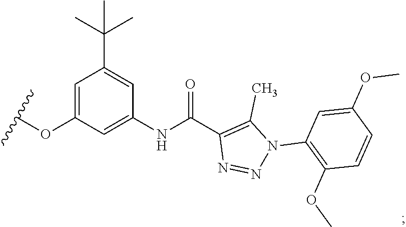

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein R⁷ is selected from hydrogen and C1-C4 alkyl, and wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl), provided that when L is —C(O)NR¹⁰—, then R² is selected from C1-C4 haloalkyl, C1-C4 alkoxy, and C1-C4 haloalkoxy, and R³ is C1-C8 alkyl, or a pharmaceutically acceptable salt thereof.

In one aspect, disclosed are compounds having a structure represented by a formula:

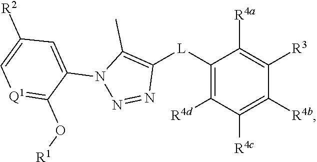

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is selected from hydrogen, halogen, C1-C8 alkyl, C1-C8 haloalkyl, C1-C8 alkoxy, and C1-C8 haloalkoxy; and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)OR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

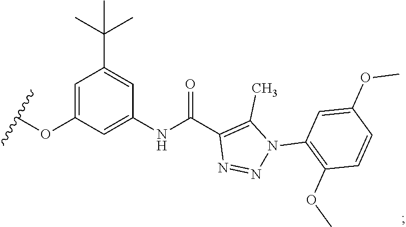

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; and wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl), provided that when L is —C(O)NR¹⁰—, then R² is selected from C1-C4 haloalkyl, C1-C4 alkoxy, and C1-C4 haloalkoxy, and R³ is C1-C8 alkyl, or a pharmaceutically acceptable salt thereof.

In one aspect, the compound is selected from:

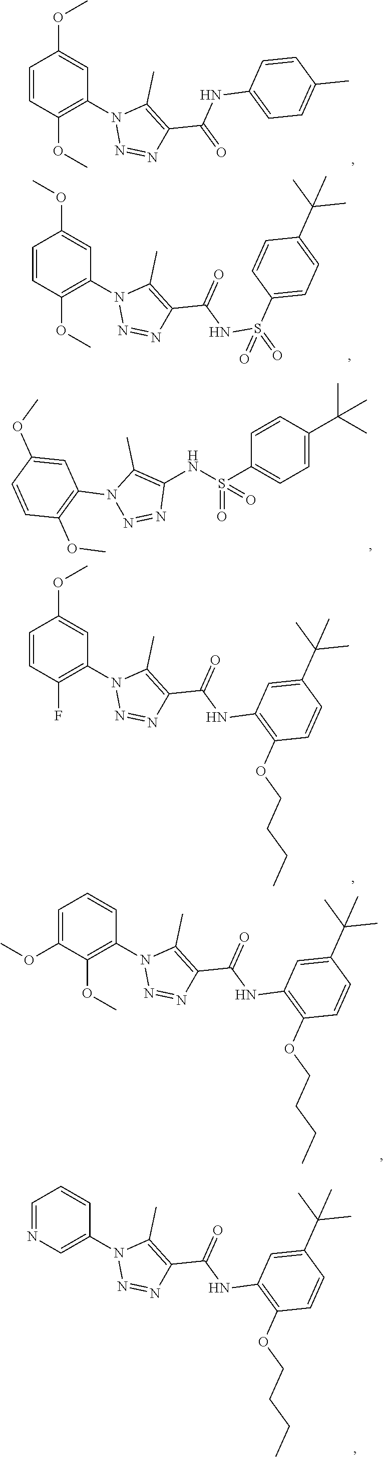

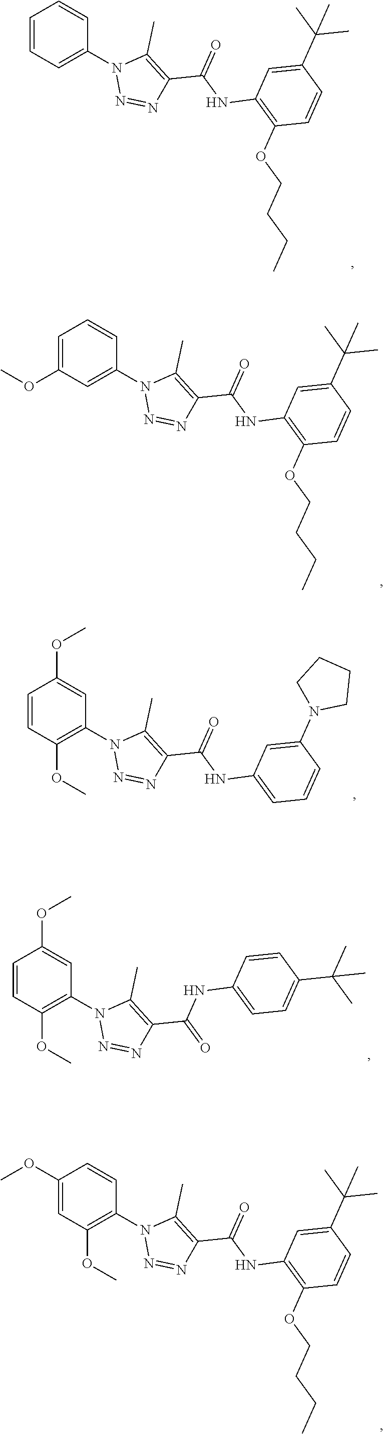

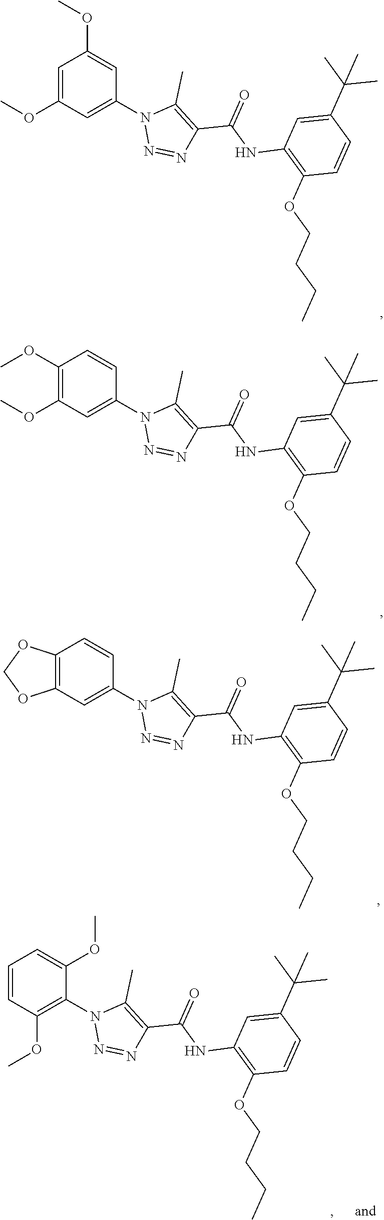

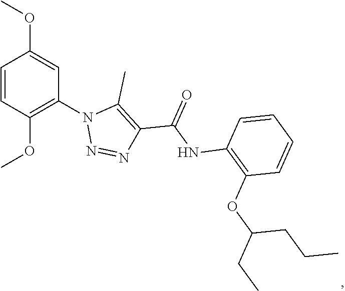 [SMILES: CCCC(CC)Oc1ccccc1NC(=O)c1nnn(-c2cc(OC)ccc2OC)c1C]

or a pharmaceutically acceptable salt thereof.

In one aspect, disclosed are compounds represented by a formula:

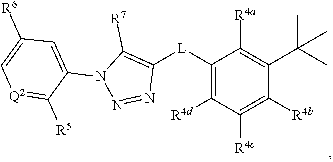

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹¹; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy, and wherein R⁷ is selected from hydrogen and Ct-C4 alkyl, or a pharmaceutically acceptable salt thereof.

In one aspect, disclosed are compounds represented by a formula:

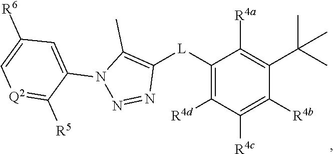

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹¹; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; and wherein R⁶ is selected from hydrogen and C1-C4 alkoxy, or a pharmaceutically acceptable salt thereof.

In one aspect, the compound is selected from:

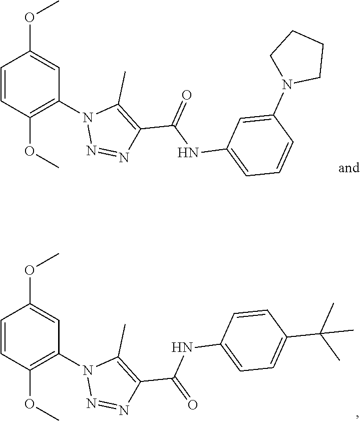 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

In one aspect, the compounds has a structure represented by a formula:

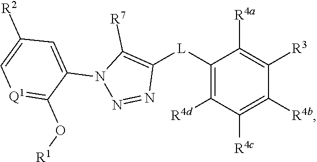

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂), OR¹⁴, —NHR⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

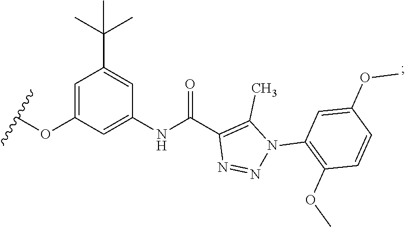

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof.

In one aspect, the compound has a structure represented by a formula:

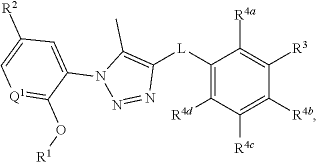

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)OR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

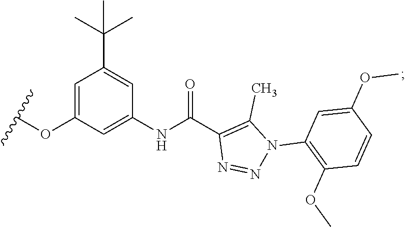

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R¹⁰ is selected from hydrogen and C1-C4 alkyl, or a pharmaceutically acceptable salt thereof.

In various aspects, the compound has a structure represented by a formula:

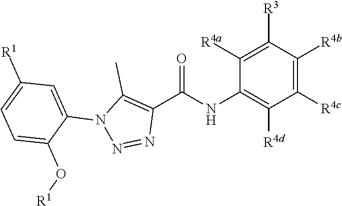

or a pharmaceutically acceptable salt thereof.

In various aspects, the compound has a structure represented by a formula:

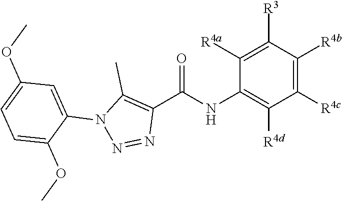

or a pharmaceutically acceptable salt thereof.

In various aspects, the compound has a structure represented by a formula:

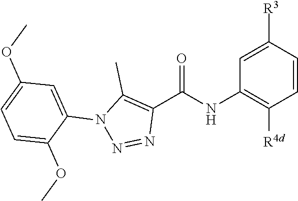

or a pharmaceutically acceptable salt thereof.

In various aspects, the compound has a structure represented by a formula:

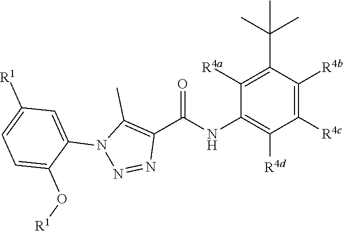

or a pharmaceutically acceptable salt thereof.

In various aspects, the compound has a structure represented by a formula:

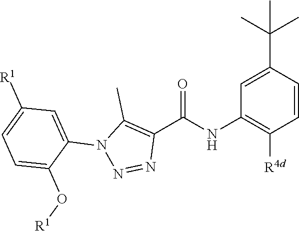

or a pharmaceutically acceptable salt thereof.

In various aspects, the compound has a structure represented by a formula:

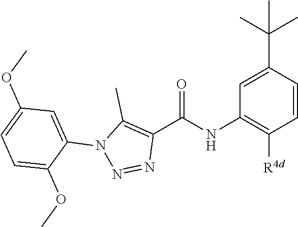

or a pharmaceutically acceptable salt thereof.

In various aspects, the compound has a structure represented by a formula:

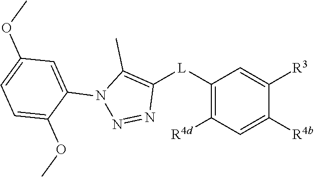

or a pharmaceutically acceptable salt thereof.

In various aspects, the compound is selected from:

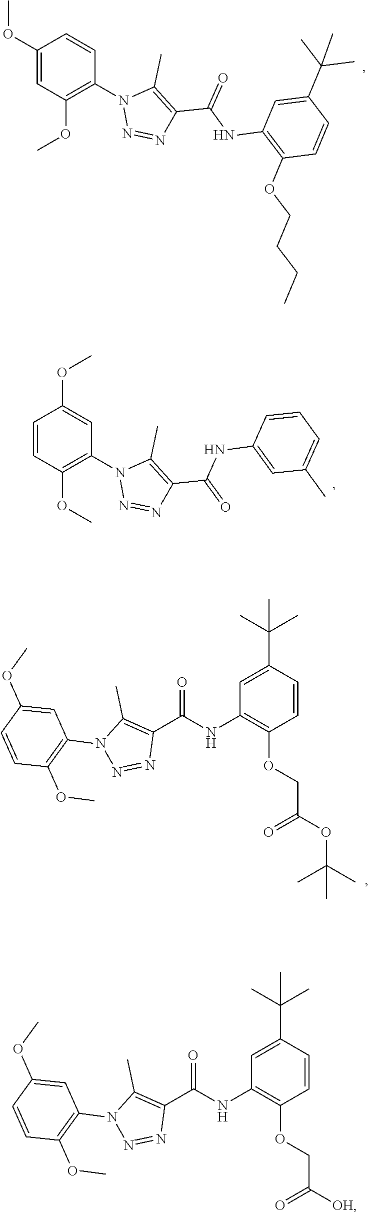 [SMILES: CCCCOc1ccc(C(C)(C)C)cc1NC(=O)c1nnn(-c2ccc(OC)cc2OC)c1C ; COc1ccc(OC)c(-n2nnc(C(=O)Nc3cccc(C)c3)c2C)c1 ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1]

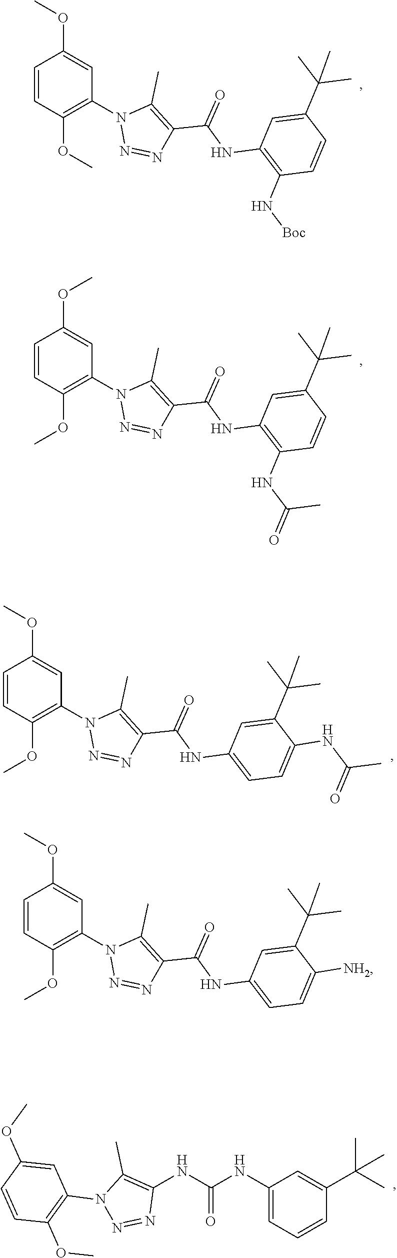 [SMILES: COc1ccc(OC)c(-n2nnc(C(=O)Nc3cc(C(C)(C)C)ccc3NC(=O)OC(C)(C)C)c2C)c1 ; COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1 ; COc1ccc(OC)c(-n2nnc(NC(=O)Nc3cccc(C(C)(C)C)c3)c2C)c1]

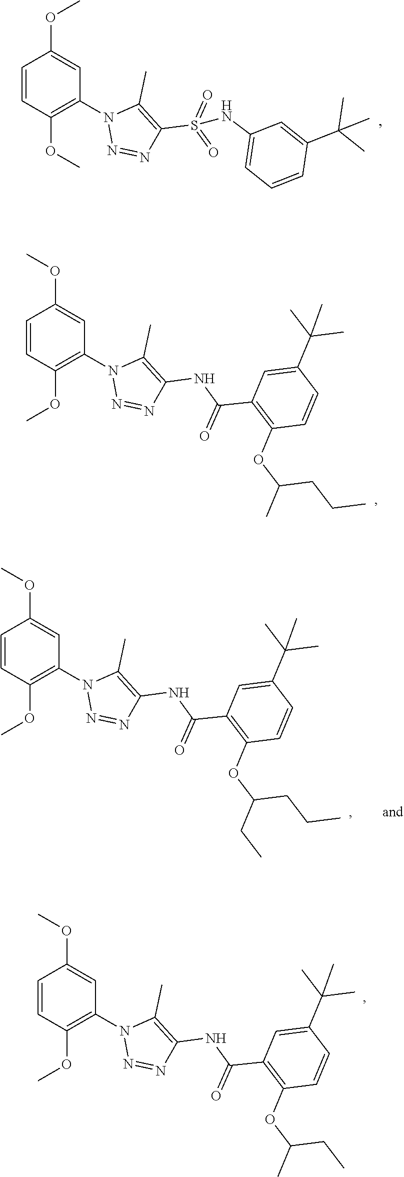 [SMILES: COc1ccc(OC)c(-n2nnc(S(=O)(=O)Nc3cccc(C(C)(C)C)c3)c2C)c1 ; CCCC(C)Oc1ccc(C(C)(C)C)cc1C(=O)Nc1nnn(-c2cc(OC)ccc2OC)c1C ; CCCC(CC)Oc1ccc(C(C)(C)C)cc1C(=O)Nc1nnn(-c2cc(OC)ccc2OC)c1C ; CCC(C)Oc1ccc(C(C)(C)C)cc1C(=O)Nc1nnn(-c2cc(OC)ccc2OC)c1C]

or a pharmaceutically acceptable salt thereof.

In various aspects, the compound has a structure represented by a formula:

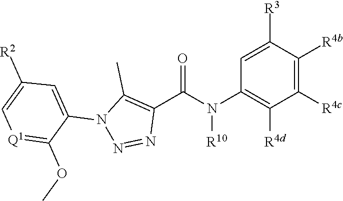

or a pharmaceutically acceptable salt thereof.

In various aspects, the compound is selected from:

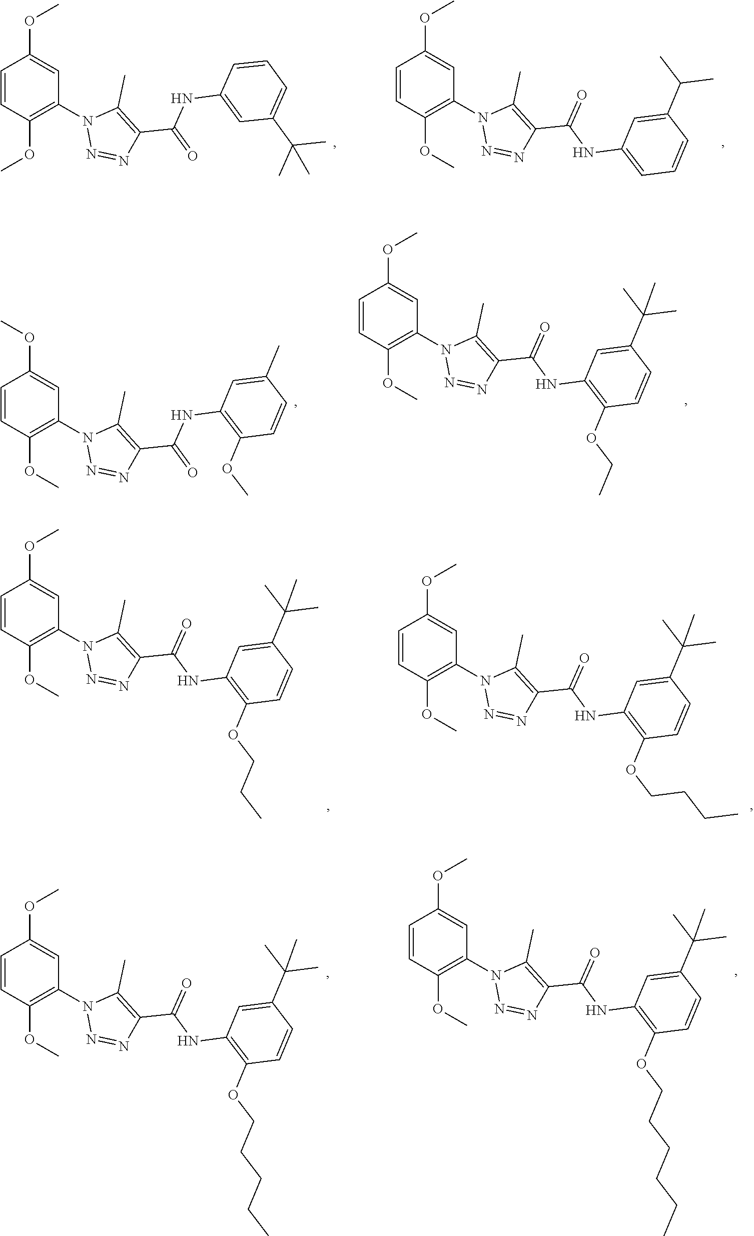

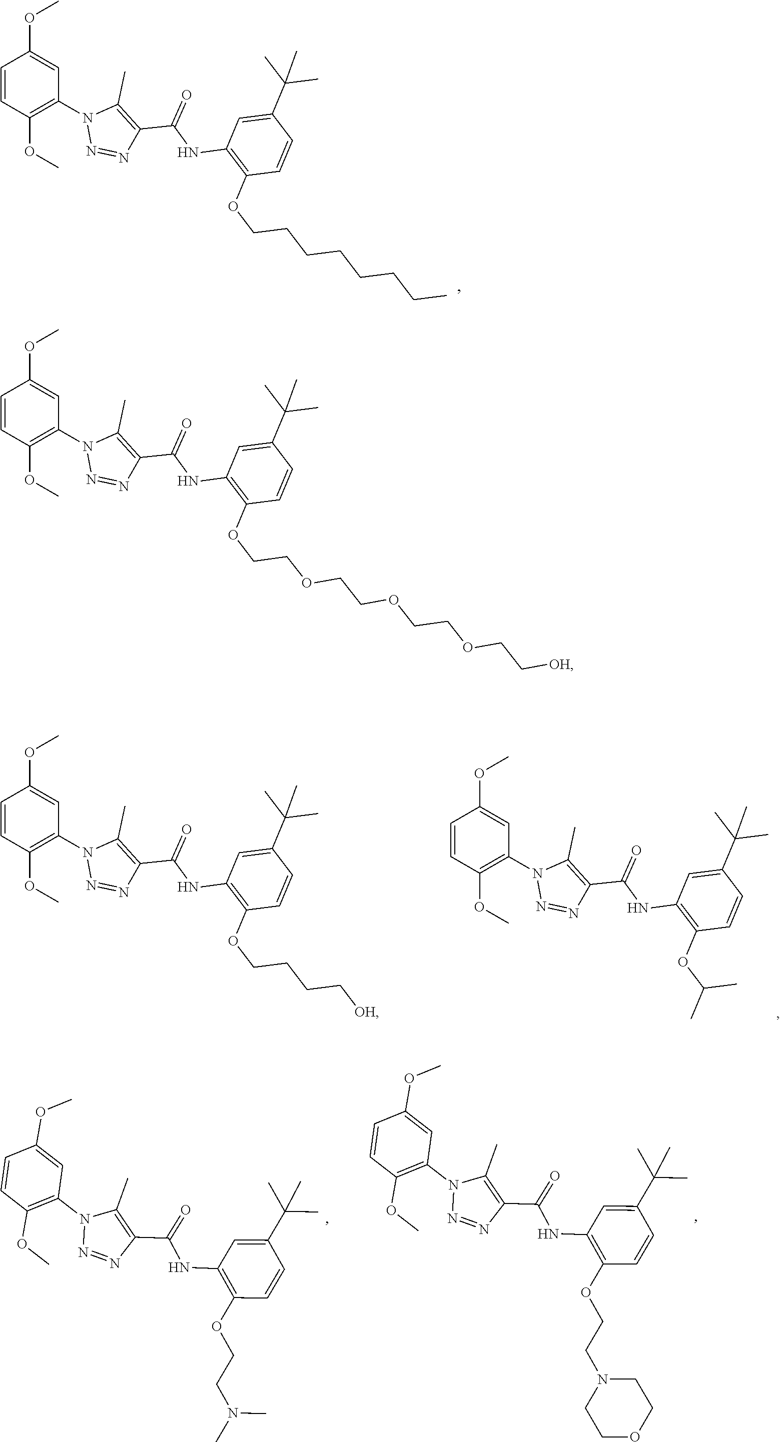 [SMILES: CCCCCCCCOc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OCCOCCOCCOCCO)c2C)c1 ; COc1ccc(OC)c(-n2nnc(C(=O)Nc3cc(C(C)(C)C)ccc3OCCCCO)c2C)c1 ; COc1ccc(OC)c(-n2nnc(C(=O)Nc3cc(C(C)(C)C)ccc3OC(C)C)c2C)c1 ; COc1ccc(OC)c(-n2nnc(C(=O)Nc3cc(C(C)(C)C)ccc3OCCN(C)C)c2C)c1 ; COc1ccc(OC)c(-n2nnc(C(=O)Nc3cc(C(C)(C)C)ccc3OCCN3CCOCC3)c2C)c1]

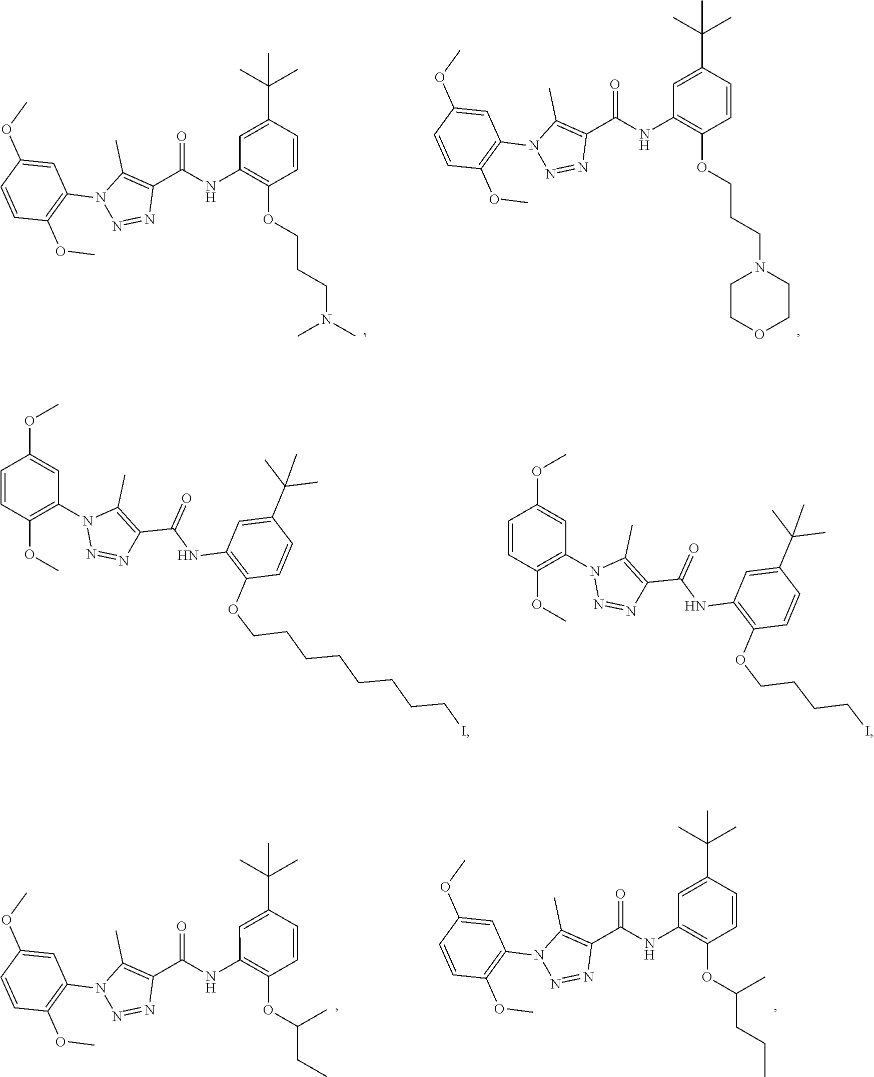 [SMILES: COc1ccc(OC)c(-n2nnc(C(=O)Nc3cc(C(C)(C)C)ccc3OCCCN(C)C)c2C)c1 ; COc1ccc(OC)c(-n2nnc(C(=O)Nc3cc(C(C)(C)C)ccc3OCCCN3CCOCC3)c2C)c1 ; COc1ccc(OC)c(-n2nnc(C(=O)Nc3cc(C(C)(C)C)ccc3OCCCCCCCCI)c2C)c1 ; COc1ccc(OC)c(-n2nnc(C(=O)Nc3cc(C(C)(C)C)ccc3OCCCCI)c2C)c1 ; CCC(C)Oc1ccc(C(C)(C)C)cc1NC(=O)c1nnn(-c2cc(OC)ccc2OC)c1C ; CCCC(C)Oc1ccc(C(C)(C)C)cc1NC(=O)c1nnn(-c2cc(OC)ccc2OC)c1C]

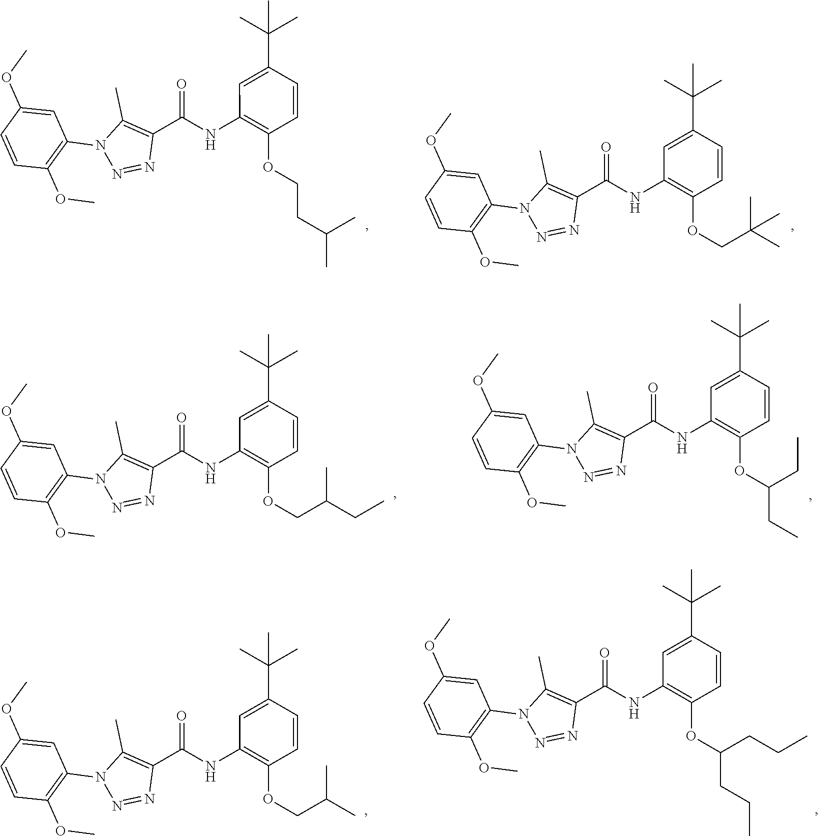 [SMILES: COc1ccc(OC)c(-n2nnc(C(=O)Nc3cc(C(C)(C)C)ccc3OCCC(C)C)c2C)c1 ; COc1ccc(OC)c(-n2nnc(C(=O)Nc3cc(C(C)(C)C)ccc3OCC(C)(C)C)c2C)c1 ; CCC(C)COc1ccc(C(C)(C)C)cc1NC(=O)c1nnn(-c2cc(OC)ccc2OC)c1C ; CCC(CC)Oc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OCC(C)C)c2C)c1 ; CCCC(CCC)Oc1ccc(C(C)(C)C)cc1NC(=O)c1nnn(-c2cc(OC)ccc2OC)c1C]

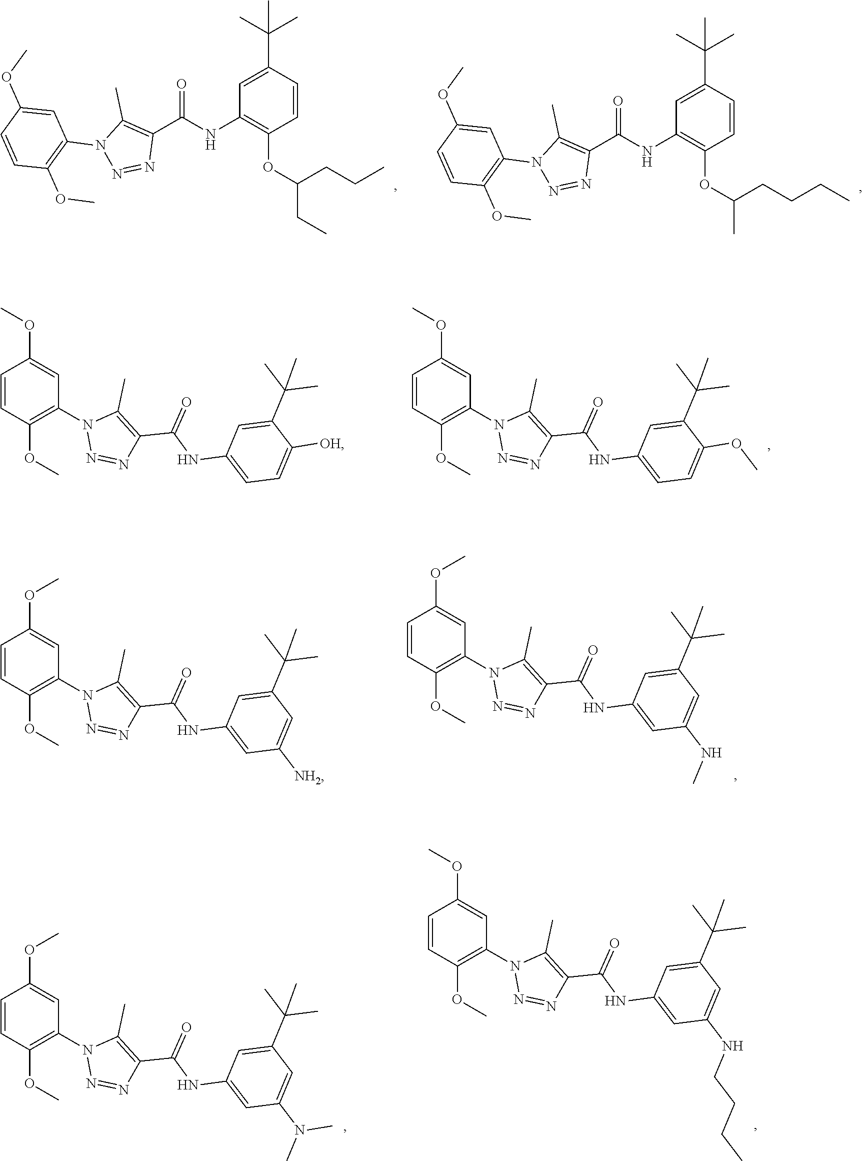

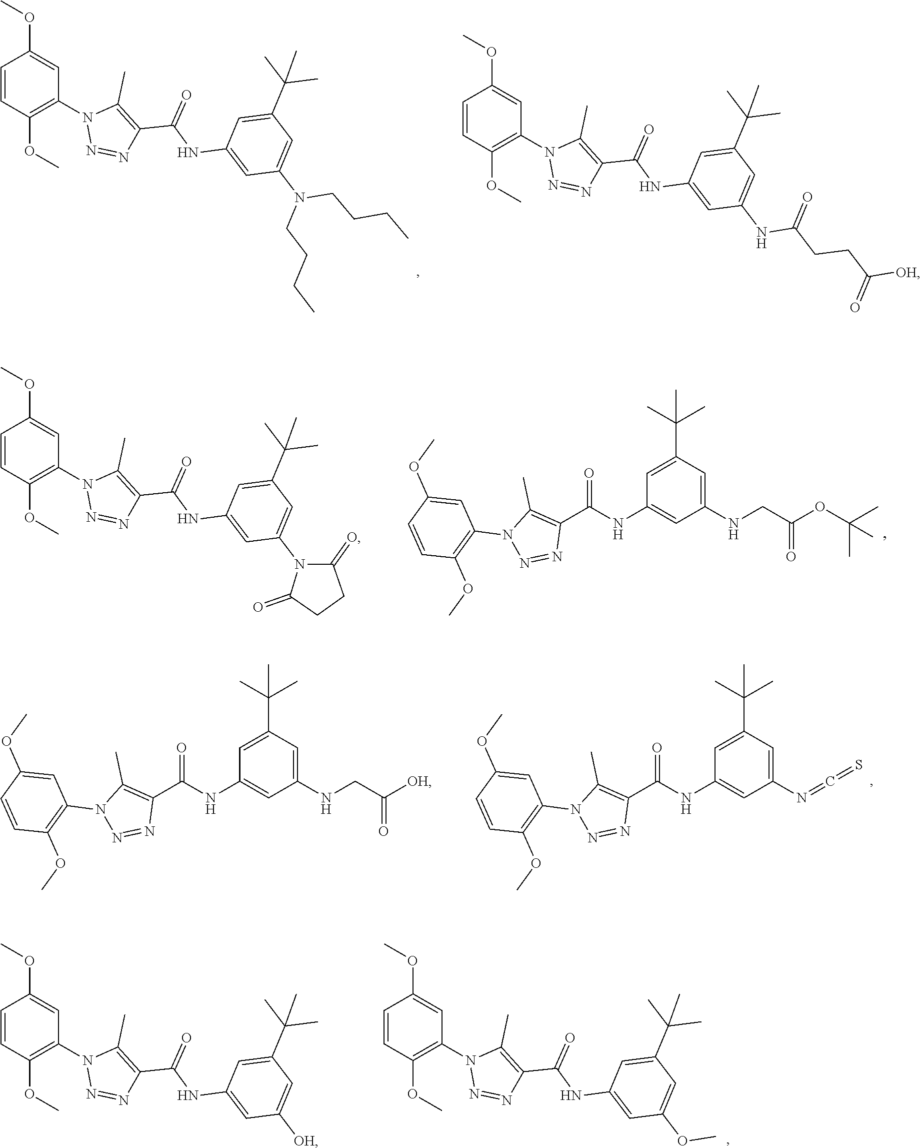

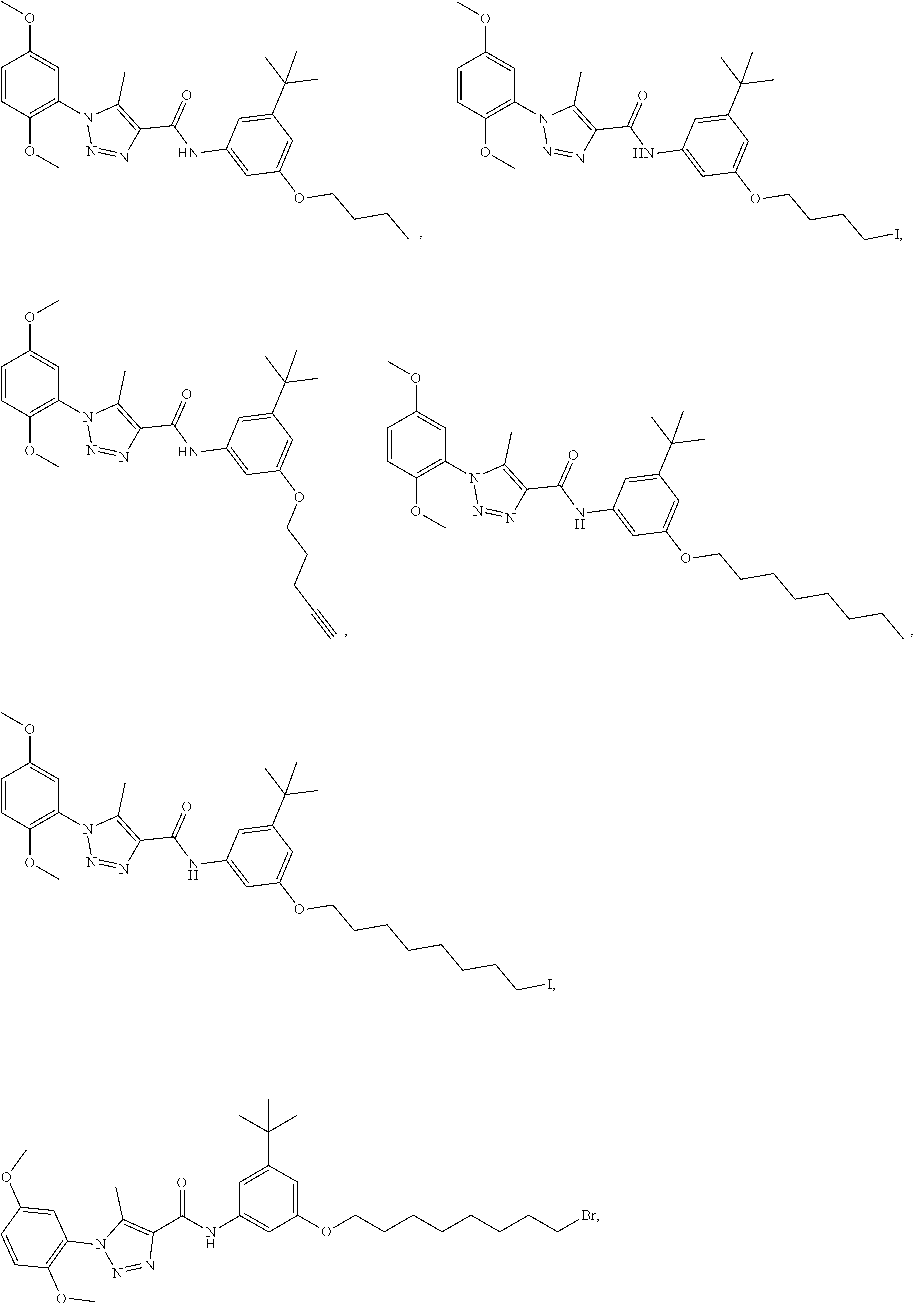 [SMILES: CCCCOc1cc(NC(=O)c2nnn(-c3cc(OC)ccc3OC)c2C)cc(C(C)(C)C)c1 ; COc1ccc(OC)c(-n2nnc(C(=O)Nc3cc(OCCCCI)cc(C(C)(C)C)c3)c2C)c1 ; C#CCCCOc1cc(NC(=O)c2nnn(-c3cc(OC)ccc3OC)c2C)cc(C(C)(C)C)c1 ; CCCCCCCCOc1cc(NC(=O)c2nnn(-c3cc(OC)ccc3OC)c2C)cc(C(C)(C)C)c1 ; COc1ccc(OC)c(-n2nnc(C(=O)Nc3cc(OCCCCCCCCI)cc(C(C)(C)C)c3)c2C)c1 ; COc1ccc(OC)c(-n2nnc(C(=O)Nc3cc(OCCCCCCCCBr)cc(C(C)(C)C)c3)c2C)c1]

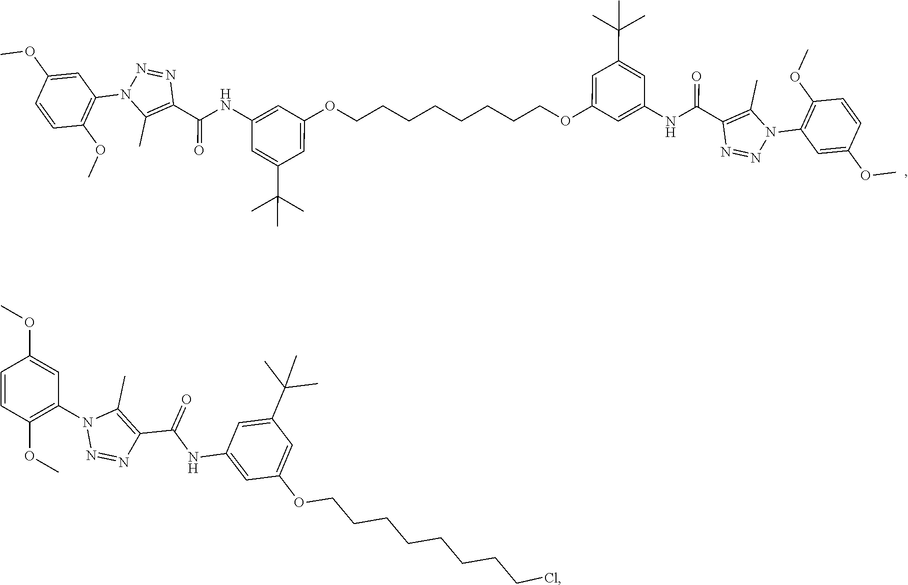

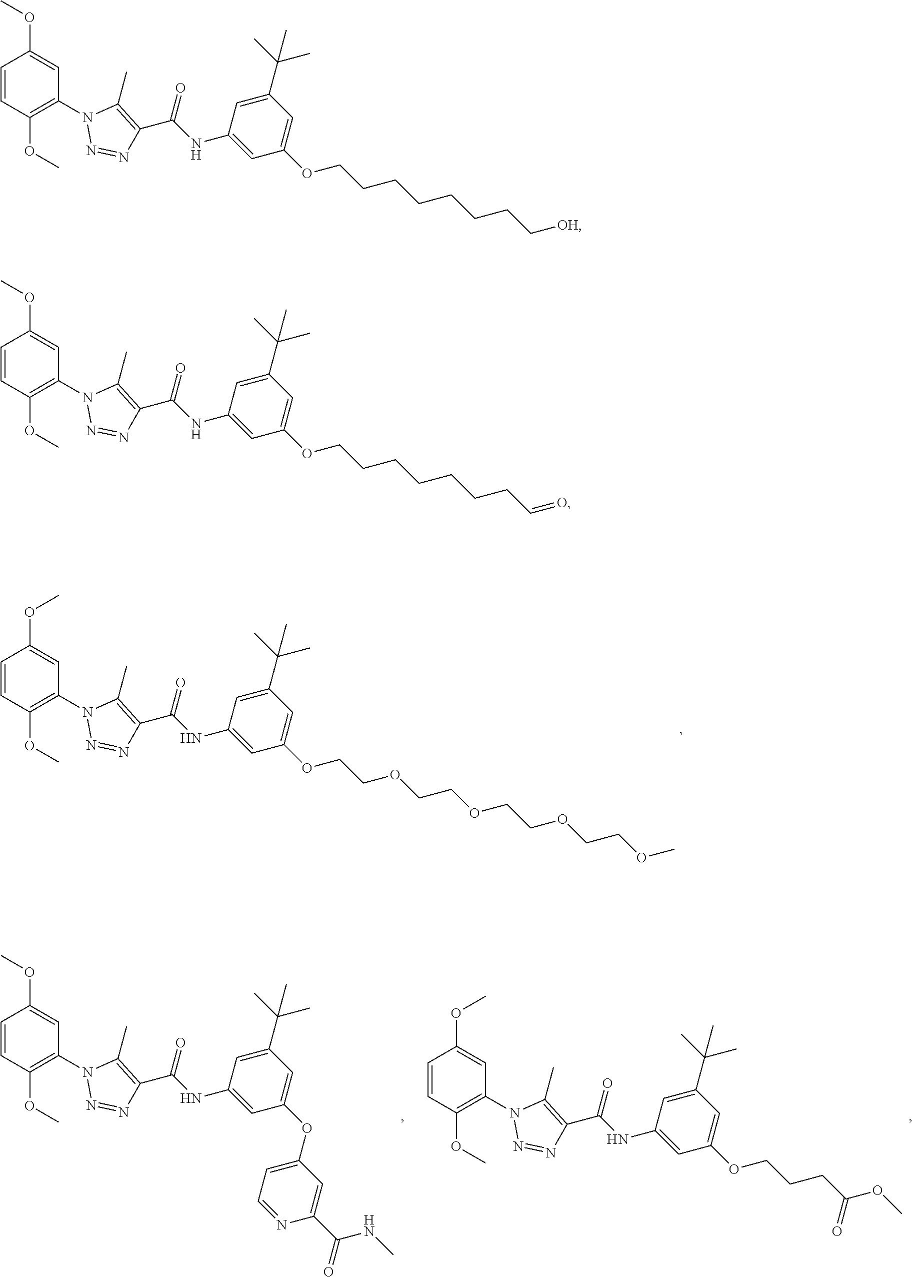 [SMILES: COc1ccc(OC)c(-n2nnc(C(=O)Nc3cc(OCCCCCCCCO)cc(C(C)(C)C)c3)c2C)c1 ; COc1ccc(OC)c(-n2nnc(C(=O)Nc3cc(OCCCCCCCC=O)cc(C(C)(C)C)c3)c2C)c1 ; COCCOCCOCCOCCOc1cc(NC(=O)c2nnn(-c3cc(OC)ccc3OC)c2C)cc(C(C)(C)C)c1 ; CNC(=O)c1cc(Oc2cc(NC(=O)c3nnn(-c4cc(OC)ccc4OC)c3C)cc(C(C)(C)C)c2)ccn1 ; COC(=O)CCCOc1cc(NC(=O)c2nnn(-c3cc(OC)ccc3OC)c2C)cc(C(C)(C)C)c1]

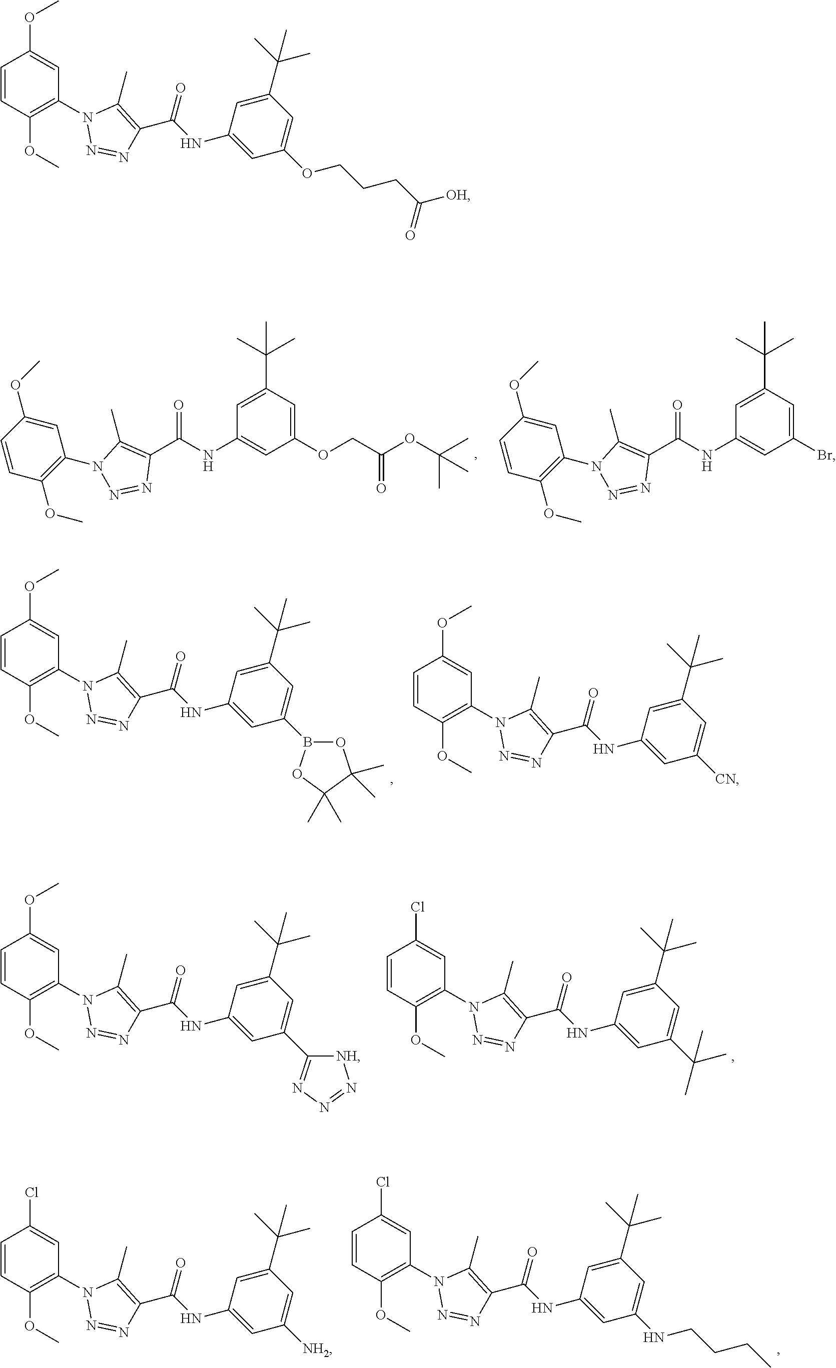

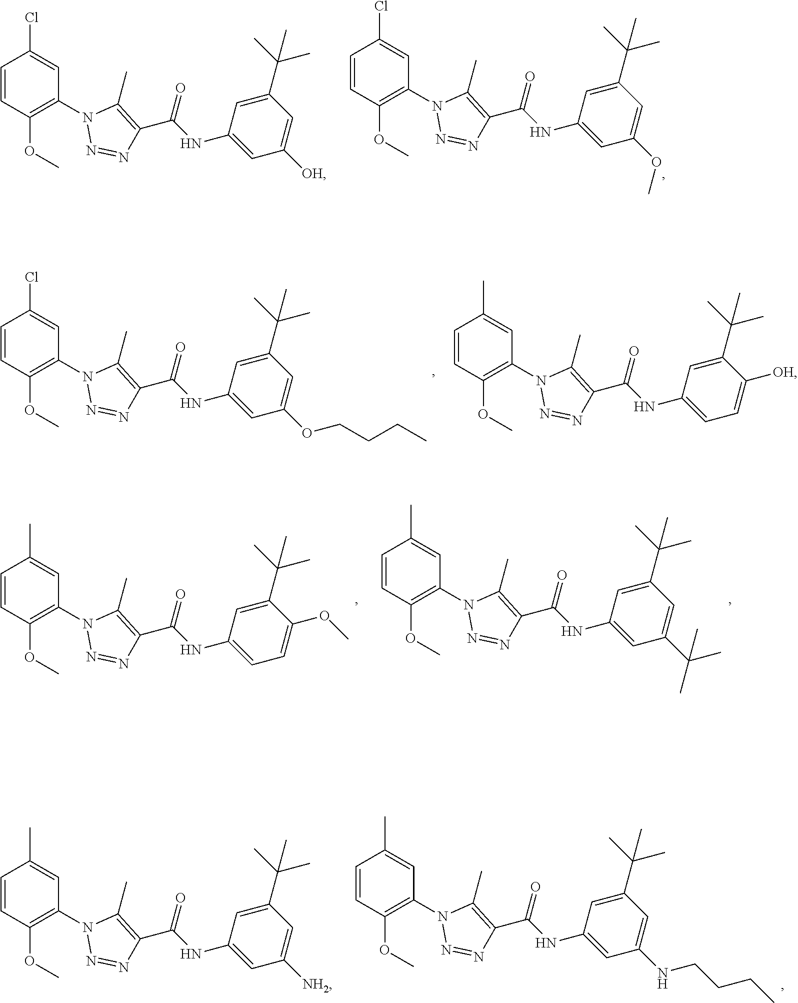

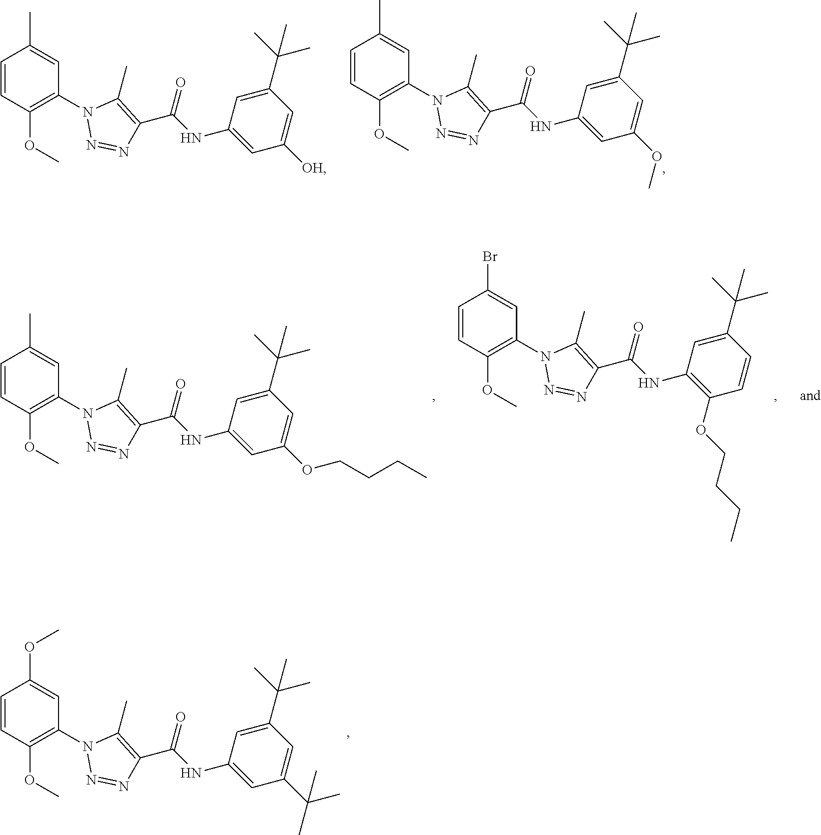

or a pharmaceutically acceptable salt thereof.

In various aspects, the compound is selected from:

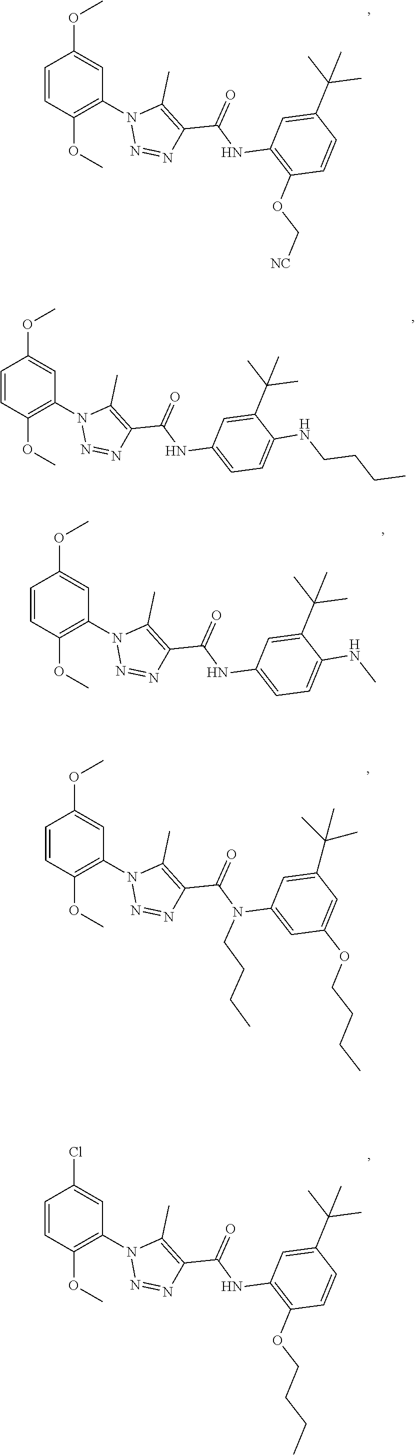 [SMILES: COc1ccc(OC)c(-n2nnc(C(=O)Nc3cc(C(C)(C)C)ccc3OCC#N)c2C)c1 ; CCCCNc1ccc(NC(=O)c2nnn(-c3cc(OC)ccc3OC)c2C)cc1C(C)(C)C ; CNc1ccc(NC(=O)c2nnn(-c3cc(OC)ccc3OC)c2C)cc1C(C)(C)C ; CCCCOc1cc(N(CCCC)C(=O)c2nnn(-c3cc(OC)ccc3OC)c2C)cc(C(C)(C)C)c1 ; CCCCOc1ccc(C(C)(C)C)cc1NC(=O)c1nnn(-c2cc(Cl)ccc2OC)c1C]

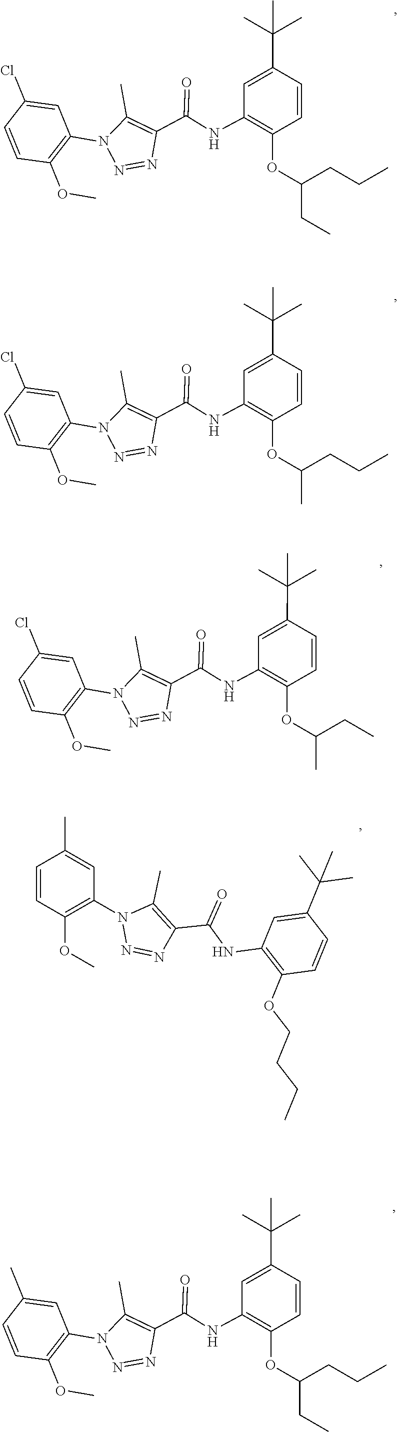 [SMILES: CCCC(CC)Oc1ccc(C(C)(C)C)cc1NC(=O)c1nnn(-c2cc(Cl)ccc2OC)c1C ; CCCC(C)Oc1ccc(C(C)(C)C)cc1NC(=O)c1nnn(-c2cc(Cl)ccc2OC)c1C ; CCC(C)Oc1ccc(C(C)(C)C)cc1NC(=O)c1nnn(-c2cc(Cl)ccc2OC)c1C ; CCCCOc1ccc(C(C)(C)C)cc1NC(=O)c1nnn(-c2cc(C)ccc2OC)c1C ; CCCC(CC)Oc1ccc(C(C)(C)C)cc1NC(=O)c1nnn(-c2cc(C)ccc2OC)c1C]

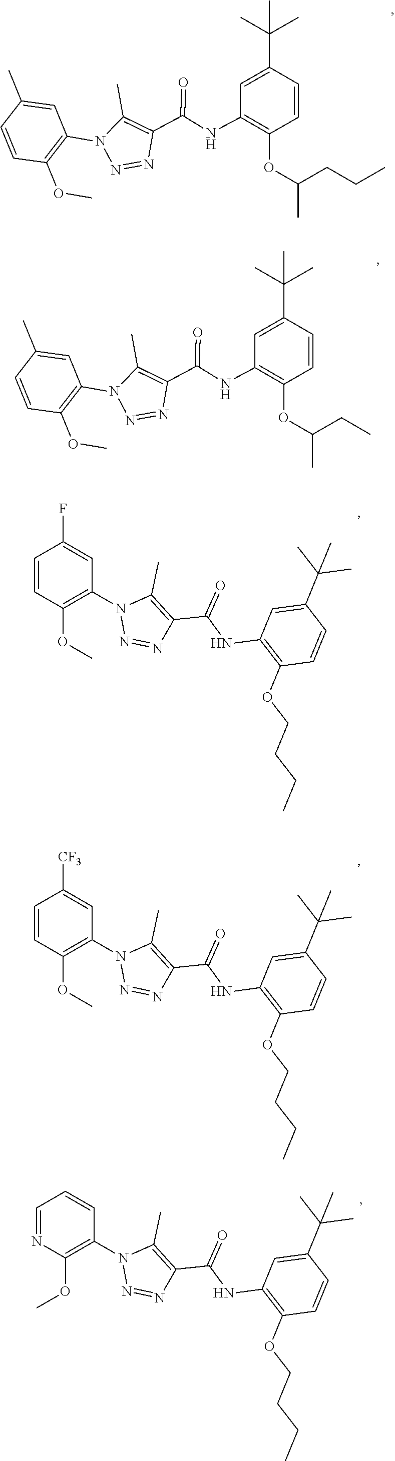

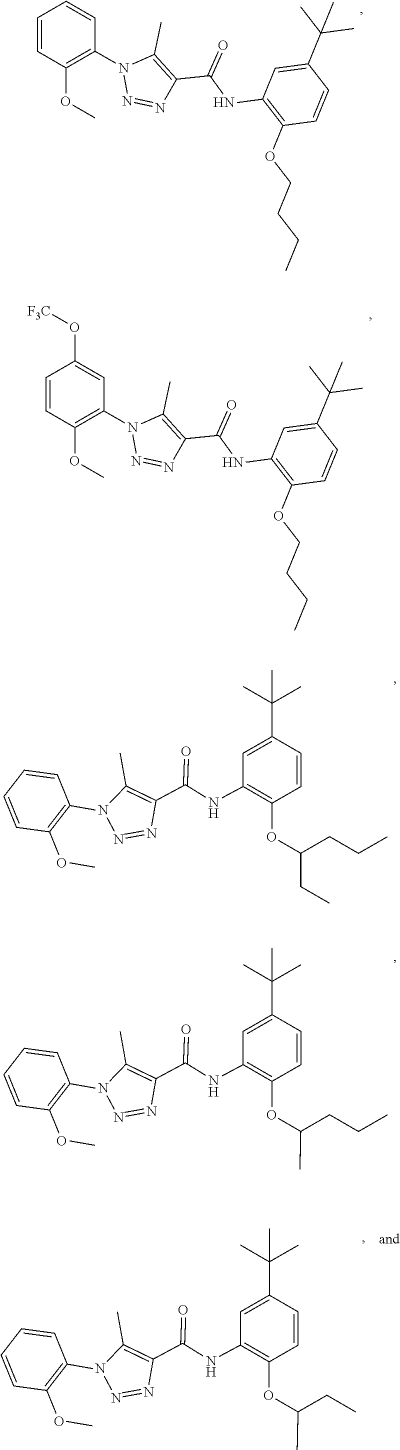

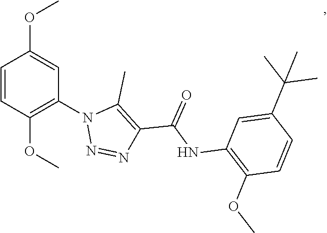 [SMILES: COc1ccc(OC)c(-n2nnc(C(=O)Nc3cc(C(C)(C)C)ccc3OC)c2C)c1]

or a pharmaceutically acceptable salt thereof.

In various aspects, the compound is not:

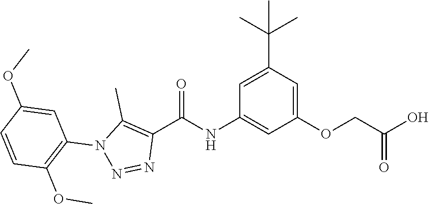 [SMILES: COc1ccc(OC)c(-n2nnc(C(=O)Nc3cc(OCC(=O)O)cc(C(C)(C)C)c3)c2C)c1]

In various aspects, the compound has a structure represented by a formula:

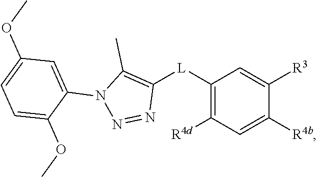

or a pharmaceutically acceptable salt thereof. In a further aspect, R³ is methyl. In a still further aspect, R³ is tert-butyl. In yet a further aspect, each of R^{4b} and R^{4d} is hydrogen. In an even further aspect, R^{4b} is selected from hydrogen, —NH₂, and —NHR¹⁵. In a still further aspect, R^{4d} is selected from hydrogen, C1-C8 alkoxy, —O—(C1-C8 alkyl)-R¹³, and —(OCH₂CH₂)ₙOR¹⁴.

In various aspects, the compound has a structure represented by a formula:

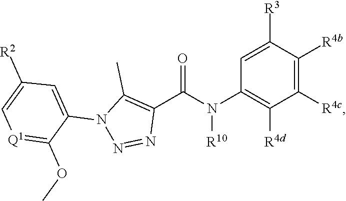

or a pharmaceutically acceptable salt thereof. In a further aspect, Q¹ is CH. In a still further aspect, R¹⁰ is hydrogen. In yet a further aspect, R³ is tert-butyl. In an even further aspect, each of R^{4b} and R^{4c} is hydrogen. In a still further aspect, R^{4d} is selected from —NH₂, —N═C═S, C1-C8 alkoxy, C1-C8 alkylamino, (C1-C8)(C1-C8) dialkylamino, —O—(C1-C8 alkyl)-R¹³, —(OCH₂CH₂), OR¹⁴, —NHR¹⁵, and Cy¹.

In various aspects, the compound is not:

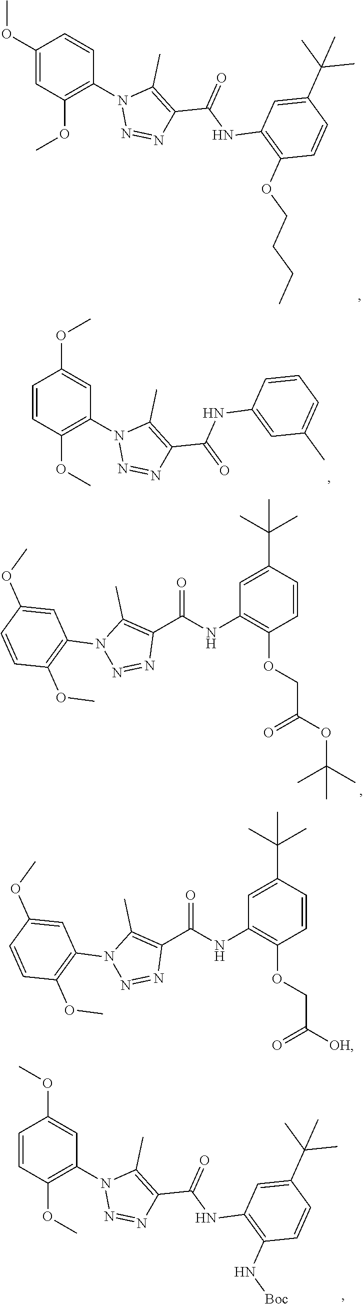

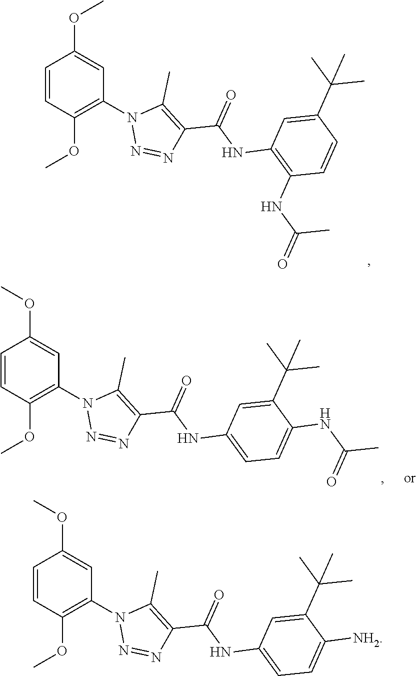 [SMILES: COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1]

In various aspects, the compound is not:

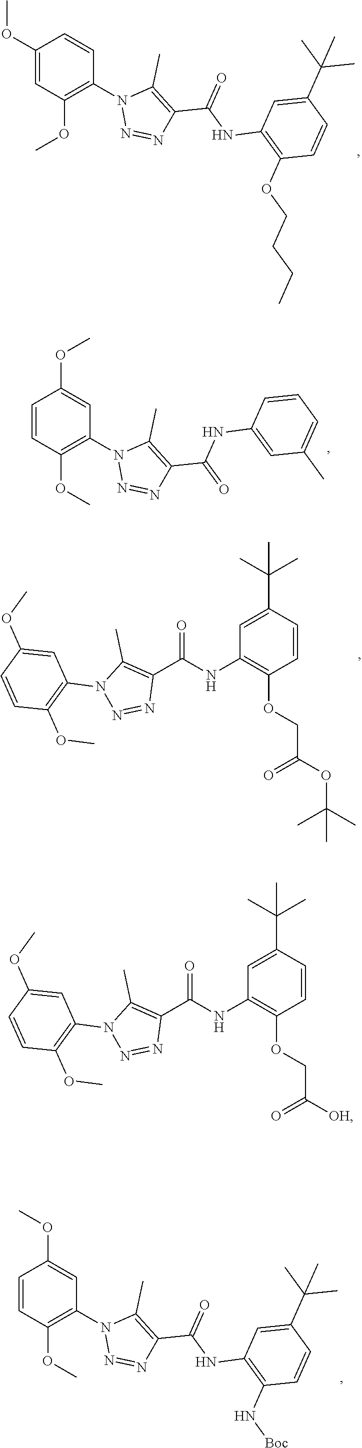 [SMILES: CCCCOc1ccc(C(C)(C)C)cc1NC(=O)c1nnn(-c2ccc(OC)cc2OC)c1C ; COc1ccc(OC)c(-n2nnc(C(=O)Nc3cccc(C)c3)c2C)c1 ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1 ; COc1ccc(OC)c(-n2nnc(C(=O)Nc3cc(C(C)(C)C)ccc3NC(=O)OC(C)(C)C)c2C)c1]

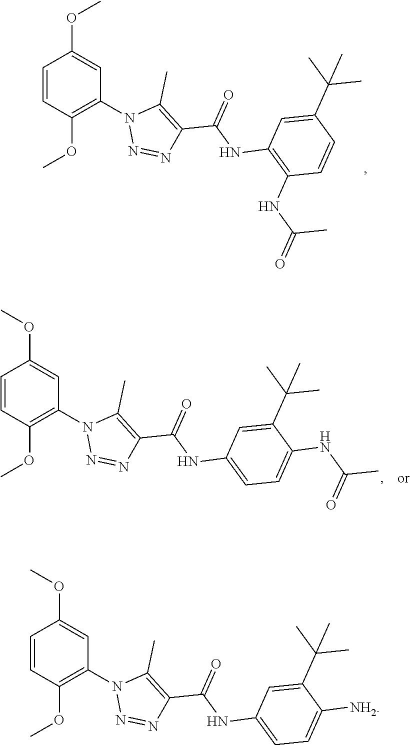 [SMILES: COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1]

In various aspects, the compound has a structure represented by a formula:

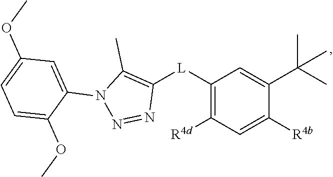

or a pharmaceutically acceptable salt thereof.

In various aspects, the compound is not:

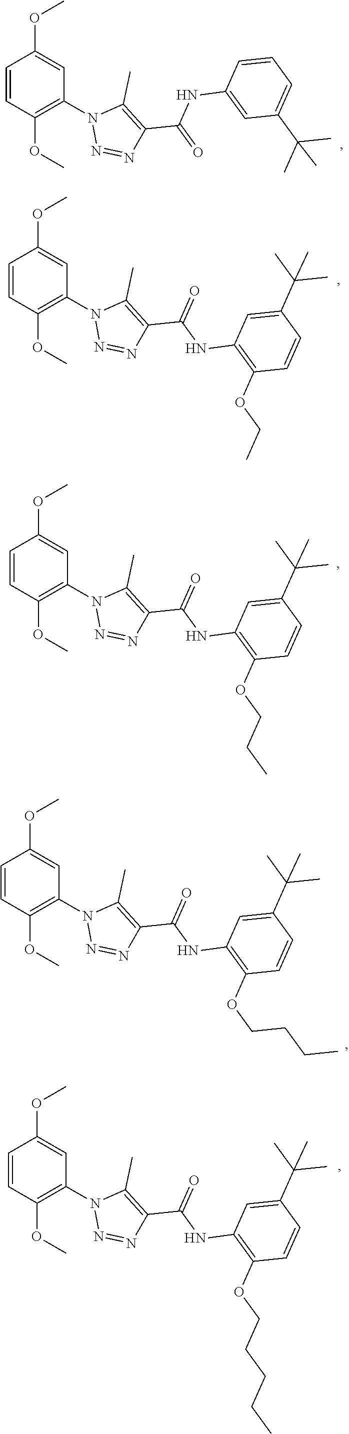

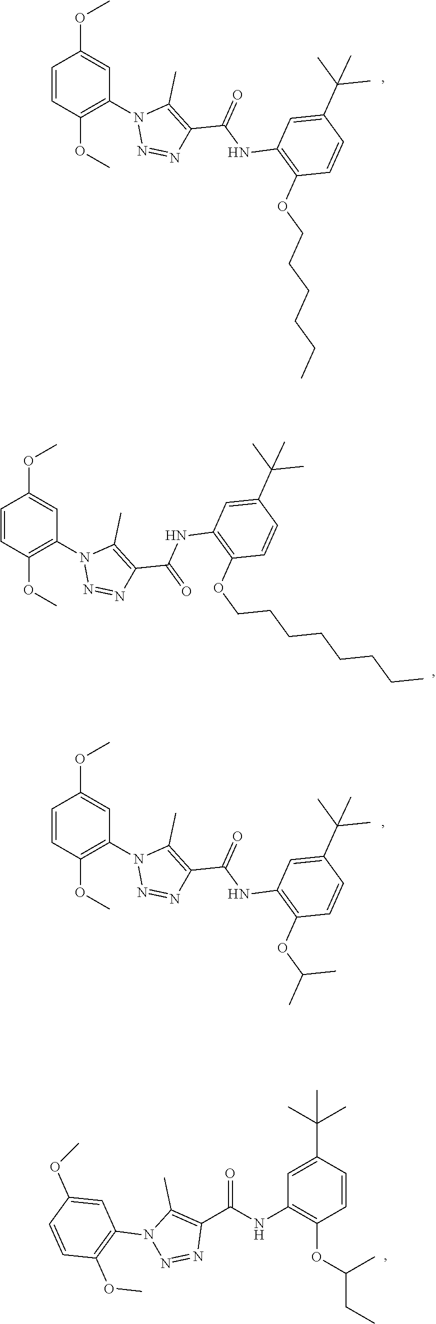 [SMILES: CCCCCCOc1ccc(C(C)(C)C)cc1NC(=O)c1nnn(-c2cc(OC)ccc2OC)c1C ; CCCCCCCCOc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OC(C)C)c2C)c1 ; CCC(C)Oc1ccc(C(C)(C)C)cc1NC(=O)c1nnn(-c2cc(OC)ccc2OC)c1C]

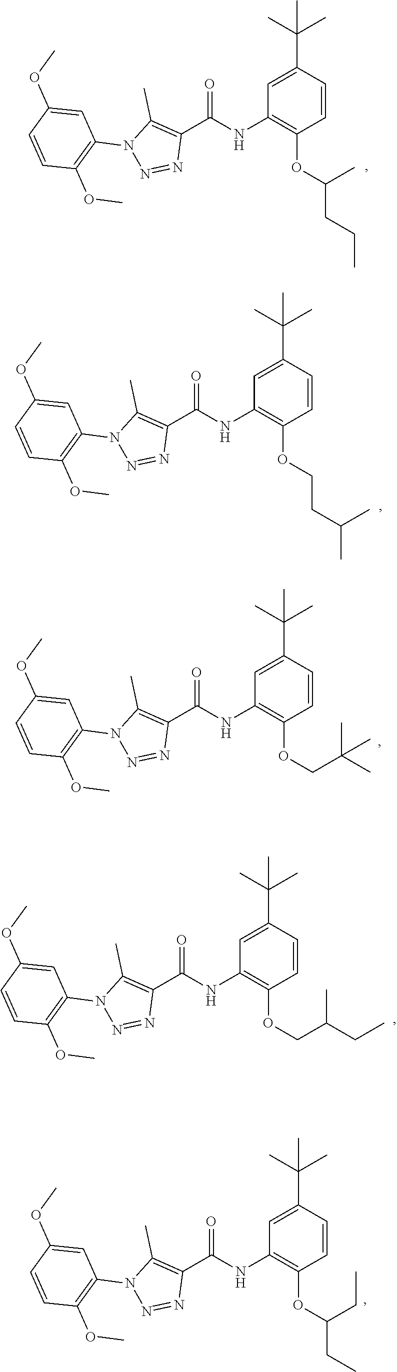 [SMILES: CCCC(C)Oc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OCCC(C)C)c2C)c1 ; COc1ccc(OC)c(-n2nnc(C(=O)Nc3cc(C(C)(C)C)ccc3OCC(C)(C)C)c2C)c1 ; CCC(C)COc1ccc(C(C)(C)C)cc1NC(=O)c1nnn(-c2cc(OC)ccc2OC)c1C ; CCC(CC)Oc1ccc(C(C)(C)C)cc1NC(=O)c1nnn(-c2cc(OC)ccc2OC)c1C]

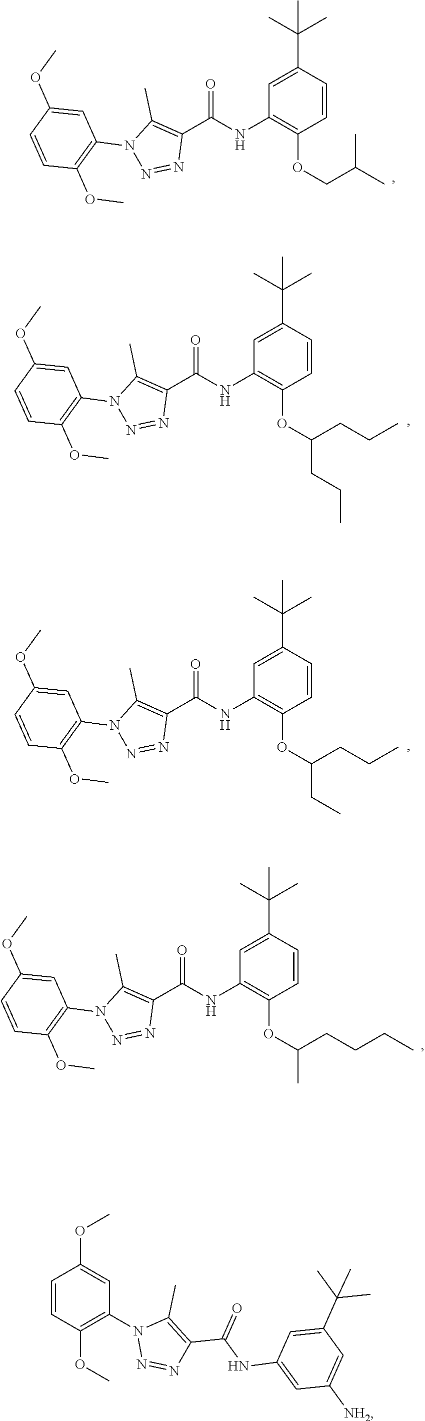 [SMILES: COc1ccc(OC)c(-n2nnc(C(=O)Nc3cc(C(C)(C)C)ccc3OCC(C)C)c2C)c1 ; CCCC(CCC)Oc1ccc(C(C)(C)C)cc1NC(=O)c1nnn(-c2cc(OC)ccc2OC)c1C ; CCCC(CC)Oc1ccc(C(C)(C)C)cc1NC(=O)c1nnn(-c2cc(OC)ccc2OC)c1C ; CCCCC(C)Oc1ccc(C(C)(C)C)cc1NC(=O)c1nnn(-c2cc(OC)ccc2OC)c1C ; COc1ccc(OC)c(-n2nnc(C(=O)Nc3cc(N)cc(C(C)(C)C)c3)c2C)c1]

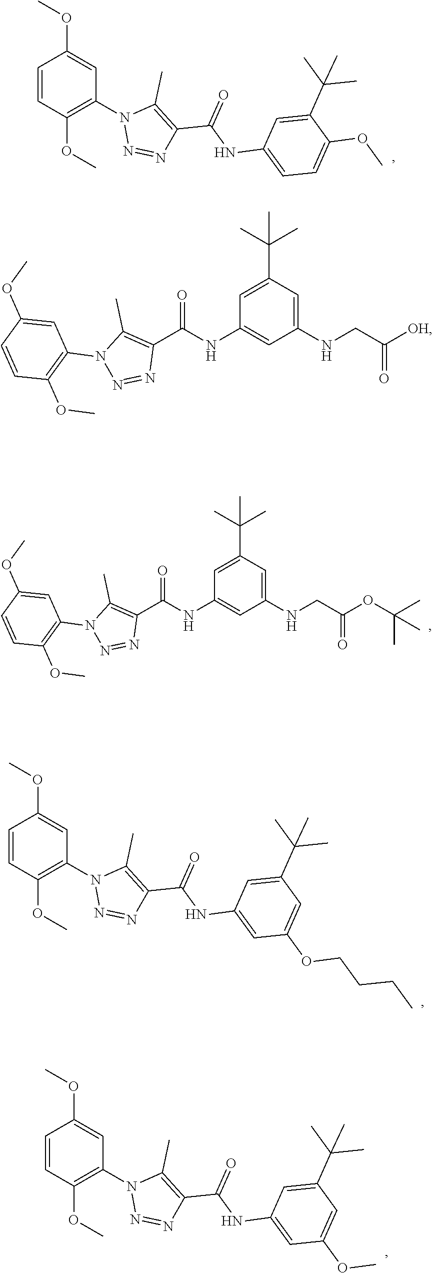

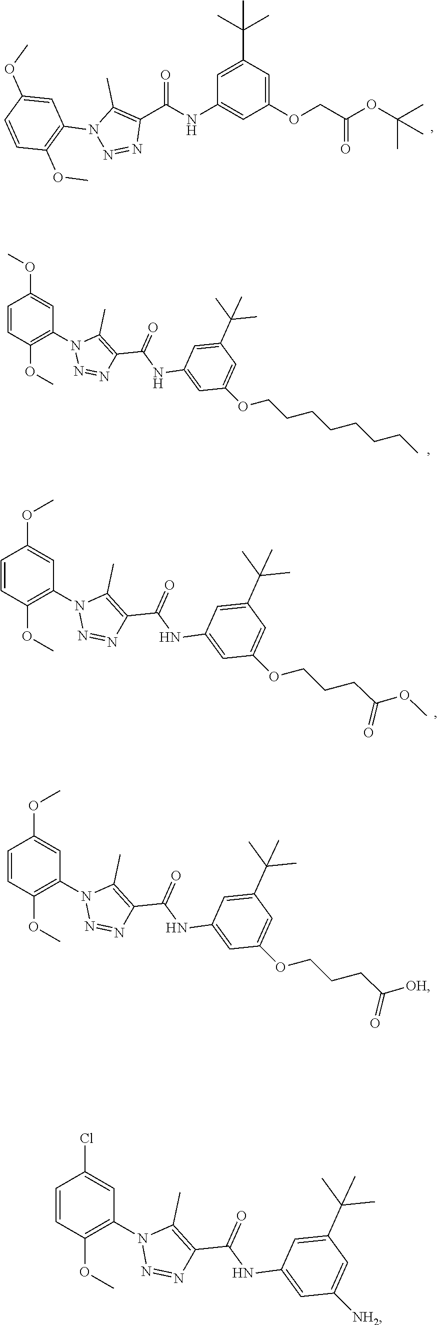 [SMILES: COc1ccc(OC)c(-n2nnc(C(=O)Nc3cc(OCC(=O)OC(C)(C)C)cc(C(C)(C)C)c3)c2C)c1 ; CCCCCCCCOc1cc(NC(=O)c2nnn(-c3cc(OC)ccc3OC)c2C)cc(C(C)(C)C)c1 ; COC(=O)CCCOc1cc(NC(=O)c2nnn(-c3cc(OC)ccc3OC)c2C)cc(C(C)(C)C)c1 ; COc1ccc(OC)c(-n2nnc(C(=O)Nc3cc(OCCCC(=O)O)cc(C(C)(C)C)c3)c2C)c1 ; COc1ccc(Cl)cc1-n1nnc(C(=O)Nc2cc(N)cc(C(C)(C)C)c2)c1C]

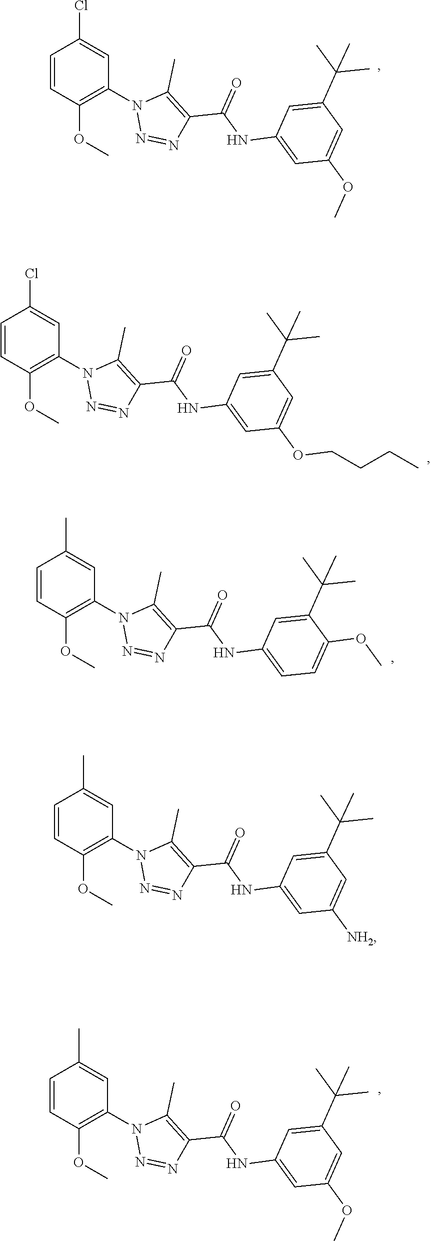 [SMILES: COc1cc(NC(=O)c2nnn(-c3cc(Cl)ccc3OC)c2C)cc(C(C)(C)C)c1 ; CCCCOc1cc(NC(=O)c2nnn(-c3cc(Cl)ccc3OC)c2C)cc(C(C)(C)C)c1 ; COc1ccc(C)cc1-n1nnc(C(=O)Nc2ccc(OC)c(C(C)(C)C)c2)c1C ; COc1ccc(C)cc1-n1nnc(C(=O)Nc2cc(N)cc(C(C)(C)C)c2)c1C ; COc1cc(NC(=O)c2nnn(-c3cc(C)ccc3OC)c2C)cc(C(C)(C)C)c1]

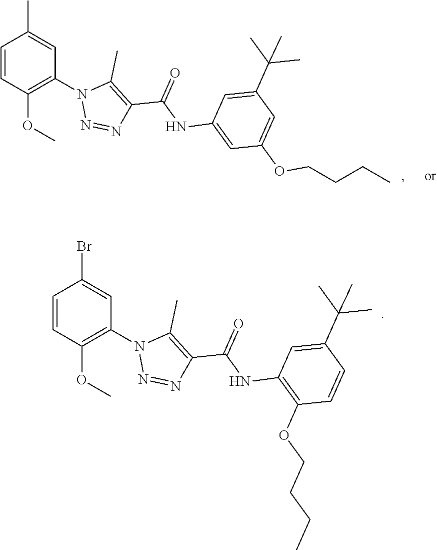 [SMILES: CCCCOc1cc(NC(=O)c2nnn(-c3cc(C)ccc3OC)c2C)cc(C(C)(C)C)c1 ; CCCCOc1ccc(C(C)(C)C)cc1NC(=O)c1nnn(-c2cc(Br)ccc2OC)c1C]

In various aspects, the compound is selected from:

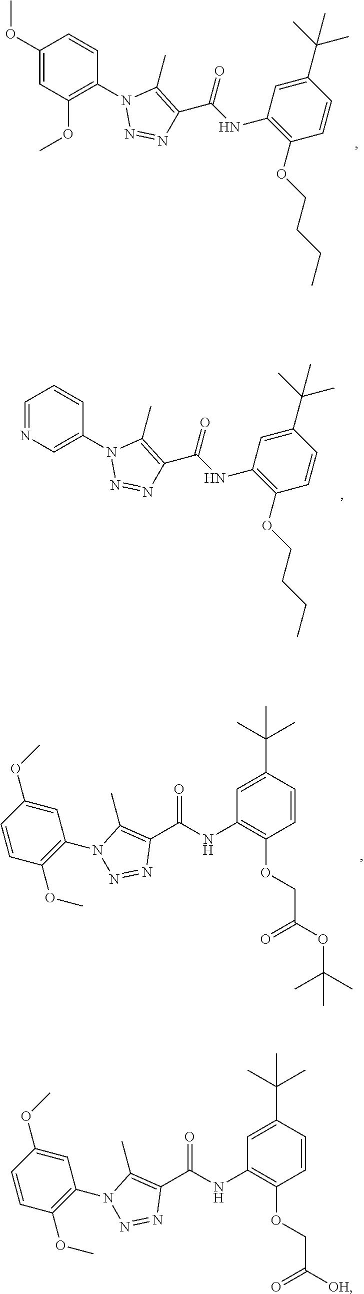 [SMILES: CCCCOc1ccc(C(C)(C)C)cc1NC(=O)c1nnn(-c2ccc(OC)cc2OC)c1C ; CCCCOc1ccc(C(C)(C)C)cc1NC(=O)c1nnn(-c2cccnc2)c1C ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1]

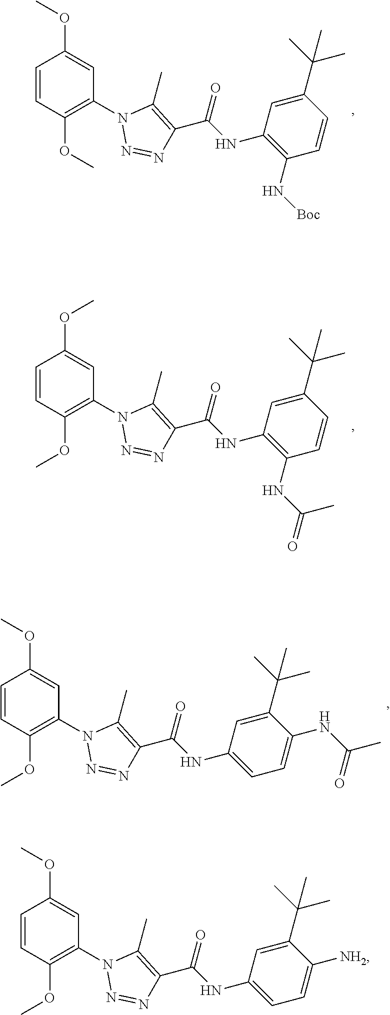 [SMILES: COc1ccc(OC)c(-n2nnc(C(=O)Nc3cc(C(C)(C)C)ccc3NC(=O)OC(C)(C)C)c2C)c1 ; COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1]

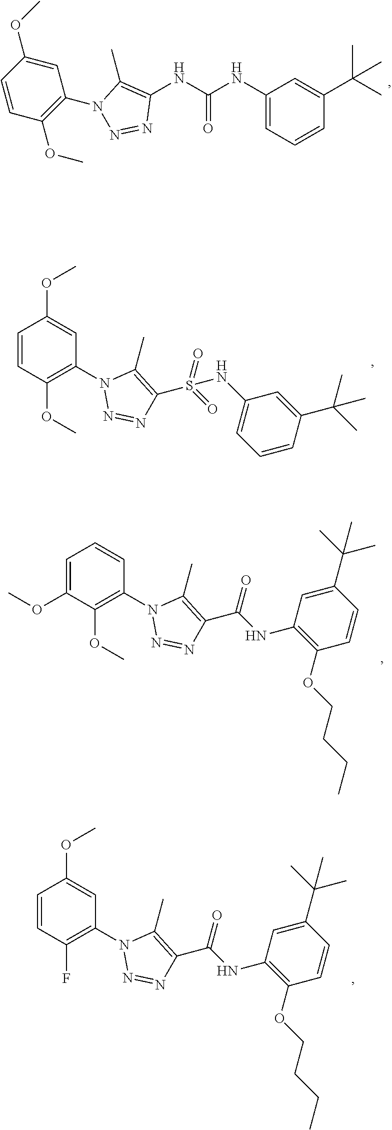

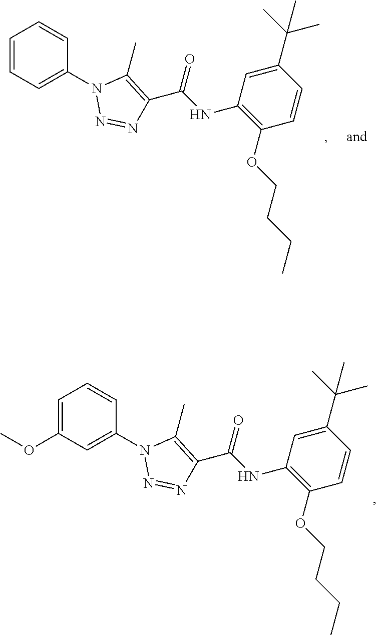

or a pharmaceutically acceptable salt thereof.

In various aspects, the compound is selected from:

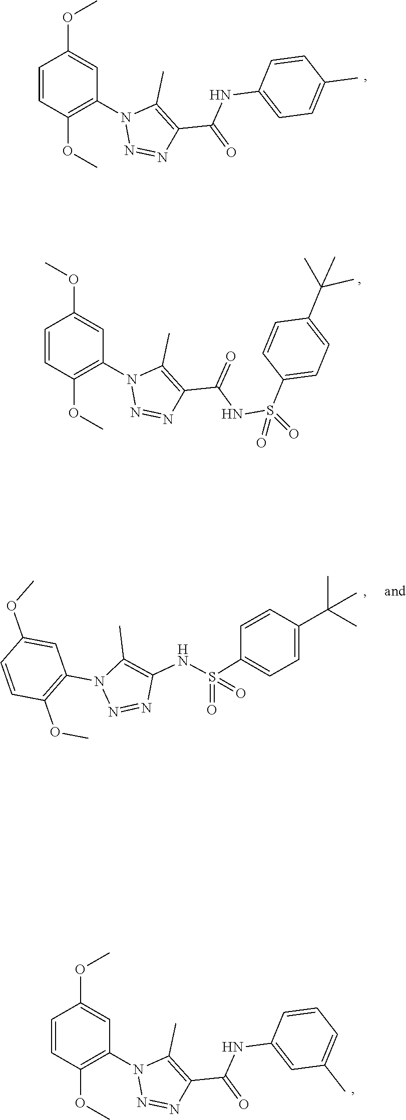 [SMILES: COc1ccc(OC)c(-n2nnc(C(=O)Nc3ccc(C)cc3)c2C)c1 ; COc1ccc(OC)c(-n2nnc(C(=O)NS(=O)(=O)c3ccc(C(C)(C)C)cc3)c2C)c1 ; COc1ccc(OC)c(-n2nnc(NS(=O)(=O)c3ccc(C(C)(C)C)cc3)c2C)c1 ; COc1ccc(OC)c(-n2nnc(C(=O)Nc3cccc(C)c3)c2C)c1]

In various aspects, n, when present, is selected from 1, 2, 3, 4, and 5. In a further aspect, n, when present, is selected from 1, 2, 3, and 4. In a still further aspect, n, when present, is selected from 1, 2, and 3. In yet a further aspect, n, when present, is selected from 1 and 2. In an even further aspect, n, when present, is selected from 2, 3, 4, and 5. In yet a further aspect, n, when present, is selected from 3, 4, and 5. In an even further aspect, n, when present, is selected from 4 and 5. In a still further aspect, n, when present, is 1. In yet a further aspect, n, when present, is 2. In an even further aspect, n, when present, is 3. In yet a further aspect, n, when present, is 4. In a still further aspect, n, when present, is 5.

a. L Groups

In one aspect, L is selected from —NR¹⁰C(O)—, —N(R¹⁰)C(O)NR¹¹—, —C(O)NR¹⁰—, —SO₂NR¹⁰—, and —NR¹⁰SO₂—. In a further aspect, L is selected from —NR¹⁰C(O)—, —N(R¹⁰)C(O)NR¹¹—, —C(O)NR¹⁰—, and —SO₂NR¹⁰—. In a still further aspect, L is selected from —NR¹⁰C(O)—, —N(R¹⁰)C(O)NR¹¹—, and —C(O)NR¹⁰—. In yet a further aspect, L is selected from —NR¹⁰C(O)— and —N(R¹⁰)C(O)NR¹¹—. In a still further aspect, L is selected from —N(R¹⁰)C(O)NR¹¹—, —C(O)NR¹⁰—, —SO₂NR¹⁰—, and —NR¹⁰SO₂—. In an even further aspect, L is selected from —C(O)NR¹⁰—, —SO₂NR¹⁰—, and —NR¹⁰SO₂—. In yet a further aspect, L is selected from —SO₂NR¹⁰— and —NR¹⁰SO₂—.

In various aspects, L is selected from —NR¹⁰C(O)—, —N(R¹⁰)C(O)NR¹¹—, and —C(O)NR¹⁰—. In a further aspect, L is selected from —NR¹⁰C(O)— and —C(O)NR¹⁰—. In yet a further aspect, L is selected from —N(R¹⁰)C(O)NR¹¹— and —C(O)NR¹⁰—. In an even further aspect, L is —N(R¹⁰)C(O)NR¹¹—. In a still further aspect, L is —N(R¹⁰)C(O)NR¹¹—. In an even further aspect, L is —C(O)NR¹⁰—.

In various aspects, L is —SO₂NR¹⁰—. In a further aspect, L is —NR¹⁰SO₂—.

In various aspects, L is selected from —C(O)NR¹⁰— and —SO₂NR¹⁰—.

b. Q¹ Groups

In one aspect. Q¹ is selected from N and CH. In a further aspect, Q¹ is N. In yet a further aspect, Q¹ is CH.

c. Q² Groups

In one aspect, Q² is selected from N and CR¹². In a further aspect, Q² is N. In yet a further aspect, Q² is CR¹².

In various aspects, Q² is selected from N and CH. In a further aspect, Q² is CH.

d. R¹ Groups

In one aspect, R¹ is C1-C4 alkyl. In a further aspect, R¹ is selected from methyl, ethyl, n-propyl, and isopropyl. In a further aspect, R¹ is selected from methyl and ethyl. In a still further aspect, R¹ is ethyl. In yet a further aspect, R¹ is methyl.

e. R² Groups

In one aspect, R² is selected from halogen, C1-C4 alkyl, C1-C4 haloalkyl, C1-C4 alkoxy, and C1-C4 haloalkoxy. In a further aspect, R² is selected from —F, —Cl, methyl, ethyl, n-propyl, isopropyl, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —OCH₃, —OCH₂CH₃, —OCH(CH₃)₂, —OCH₂CH₂CH₃, —OCF₃, —OCHF₂, —OCH₂F, —OCH₂CF₃, —OCH₂CHF₂, OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CH(Cl)CH₃. In yet a further aspect, R² is selected from —F, —Cl, methyl, ethyl, —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —CH(Cl)CH₃, —OCH₃, —OCH₂CH₃, —OCH(CH₃)₂, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCH(Cl)CH₃. In an even further aspect, R² is selected from —F, —Cl, methyl, —CF₃, —CHF₂, —CH₂F, —CCl₃, —CHCl₂, —CH₂Cl, —OCH₃, —OCF₃, —OCHF₂, —OCH₂F, —OCCl₃, —OCHCl₂, and —OCH₂Cl.

In one aspect, R² is selected from C1-C4 haloalkyl, C1-C4 alkoxy, and C1-C4 haloalkoxy. In a further aspect, R² is selected from —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —OCH₃, —OCH₂CH₃, —OCH(CH₃)₂, —OCH₂CH₂CH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CH(Cl)CH₃. In yet a further aspect, R² is selected from —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —CH(Cl)CH₃, —OCH₃, —OCH₂CH₃, —OCH(CH₃)₂, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCH(Cl)CH₃. In an even further aspect, R² is selected from —CF₃, —CHF₂, —CH₂F, —CCl₃, —CHCl₂, —CH₂Cl, —OCH₃, —OCF₃, —OCHF₂, —OCH₂F, —OCCl₃, —OCHCl₂, and —OCH₂Cl.

In various aspects, R² is selected from halogen, C1-C4 alkyl, and C1-C4 alkoxy. In a further aspect, R² is selected from —F, —Cl, methyl, ethyl, n-propyl, isopropyl, —OCH₃, —OCH₂CH₃, —OCH(CH₃)₂, and —OCH₂CH₂CH₃. In yet a further aspect, R² is selected from —F, —Cl, methyl, ethyl, —OCH₃, and —OCH₂CH₃. In a still further aspect, R² is selected from —F, —Cl, methyl, and —OCH₃.

In various aspects, R² is selected from C1-C4 alkyl and C1-C4 alkoxy. In a further aspect, R² is selected from methyl, ethyl, n-propyl, isopropyl, —OCH₃, —OCH₂CH₃, —OCH(CH₃)₂, and —OCH₂CH₂CH₃. In yet a further aspect, R² is selected from methyl, ethyl, —OCH₃, and —OCH₂CH₃. In a still further aspect, R² is selected from methyl and —OCH₃.

In various aspects, R² is C1-C4 alkoxy. In a further aspect, R² is selected from —OCH₃, —OCH₂CH₃, —OCH(CH₃)₂, and —OCH₂CH₂CH₃. In a still further aspect, R² is selected from —OCH₃ and —OCH₂CH₃. In yet a further aspect, R² is —OCH₃.

In various aspects, R² is selected from halogen, C1-C4 haloalkyl, and C1-C4 haloalkoxy. In a further aspect, R² is selected from —F, —Cl, —CF₃, —CHF₂, —CH₂F, —CH₂CF₃, CH₂CHF₂, —CH₂CH₂F, —CH(F)CH₃, —CH(CH₃)CF₃, —CH(CH₃)CHF₂, —CH(CH₃)CH₂F, CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CH(Cl)CH₃. In yet a further aspect, R² is selected from —F, —Cl, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH(Cl)CH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCH(Cl)CH₃. In a still further aspect, R² is selected from —F, —Cl, —CF₃, —CHF₂, —CH₂F, —CCl₃, —CHCl₂, —CH₂Cl, OCF₃, —OCHF₂, —OCH₂F, —OCCl₃, —OCHCl₂, and —OCH₂Cl.

In various aspects, R² is selected from C1-C4 haloalkyl and C1-C4 haloalkoxy. In a further aspect, R² is selected from —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CH(Cl)CH₃. In yet a further aspect, R² is selected from —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CH(Cl)CH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCH(Cl)CH₃. In a still further aspect, R³ is selected from —CF₃, —CHF₂, —CH₂F, —CCl₃, —CHCl₂, —CH₂Cl, —OCF₃, —OCHF₂, —OCH₂F, —OCCl₃, —OCHCl₂, and —OCH₂C1. In yet a further aspect, R² is selected from —CF₃ and —OCF₃.

In various aspects, R² is C1-C4 alkyl. In a still further aspect, R² is selected from methyl, ethyl, n-propyl, and isopropyl. In yet a further aspect, R² is selected from methyl and ethyl. In a still further aspect, R² is methyl.

In various aspects, R² is halogen. In a further aspect, R² is selected from —F, —C1, and —Br. In a still further aspect, R² is selected from —F and —Cl. In yet a further aspect, R² is —Cl. In an even further aspect, R² is —F.

f. R³ Groups

In one aspect, R³ is selected from hydrogen, halogen, C1-C8 alkyl, C1-C8 haloalkyl, C1-C8 alkoxy, C1-C8 haloalkoxy, —CO₂(C1-C4 alkyl), and —C(O)Cy². In a further R³ is selected from hydrogen, —F, —Cl, methyl, ethyl, n-propyl, isopropyl, —OCH₃, —OCH₂CH₃, —OCH(CH₃)₂, —OCH₂CH₂CH₃, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, —OCH₂CH(Cl)CH₃, —CO₂CH₃, —CO₂CH₂CH₃, —CO₂CH₂CH₂CH₃, —CO₂CH(CH₃)₂, and —C(O)Cy². In a still further aspect, R³ is selected from hydrogen, —F, —Cl, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CH(Cl)CH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —CO₂CH₃, —CO₂CH₂CH₃, and —C(O)Cy². In yet a further aspect, R³ is selected from hydrogen, —F, —Cl, methyl, —OCH₃, —CF₃, —CHF₂, —CH₂F, —CCl₃, —CHCl₂, —CH₂Cl, —OCF₃, —OCHF₂, —OCH₂F, —OCCl₃, —OCHCl₂, —OCH₂Cl, —CO₂CH₃, and —C(O)Cy².

In one aspect, R³ is selected from hydrogen, halogen, C1-C8 alkyl, C1-C8 haloalkyl, C1-C8 alkoxy, and C1-C8 haloalkoxy. In a further aspect, R³ is selected from hydrogen, halogen, C1-C4 alkyl, C1-C4 haloalkyl, C1-C4 alkoxy, and C1-C4 haloalkoxy. In yet a further aspect, R³ is selected from hydrogen, —F, —Cl, methyl, ethyl, n-propyl, isopropyl, —OCH₃, —OCH₂CH₃, —OCH(CH₃)₂, —OCH₂CH₂CH₃, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CH(Cl)CH₃. In a still further aspect, R³ is selected from hydrogen, —F, —Cl, methyl, ethyl, —OCH₃, —OCH₂CH₃, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, CH₂CHCl₂, —CH₂CH₂Cl, —CH(Cl)CH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCH(Cl)CH₃. In yet a further aspect, R³ is selected from hydrogen, —F, —Cl, methyl, —OCH₃, —CF₃, —CHF₂, —CH₂F, —CCl₃, —CHCl₂, —CH₂Cl, —OCF₃, —OCHF₂, —OCH₂F, —OCCl₃, —OCHCl₂, and —OCH₂Cl.

In one aspect, R³ is C1-C8 alkyl. In a further aspect, R³ is C1-C4 alkyl. In yet a further aspect, R³ is selected from methyl, ethyl, n-propyl, and isopropyl. In yet a further aspect, R³ is selected from methyl and ethyl. In a still further aspect, R³ is methyl.

In one aspect, R³ is selected from C1-C8 alkyl, —CO₂(C1-C4 alkyl), and —C(O)Cy². In a further R³ is selected from methyl, ethyl, n-propyl, isopropyl, —CO₂CH₃, —CO₂CH₂CH₃, —CO₂CH₂CH₂CH₃, —CO₂CH(CH₃)₂, and —C(O)Cy². In a still further aspect, R³ is selected from methyl, ethyl, —CO₂CH₃, —CO₂CH₂CH₃, and —C(O)Cy². In yet a further aspect, R³ is selected from methyl, —CO₂CH₃, and —C(O)Cy².

In one aspect, R³ is selected from tert-butyl and —C(O)Cy².

In various aspects, R³ is selected from —CO₂(C1-C4 alkyl) and —C(O)Cy². In a further aspect, R³ is selected from —CO₂CH₃, —CO₂CH₂CH₃, —CO₂CH₂CH₂CH₃, —CO₂CH(CH₃)₂, and —C(O)Cy². In a still further aspect, R³ is selected from —CO₂CH₃, —CO₂CH₂CH₃, and —C(O)Cy². In yet a further aspect, R³ is selected from —CO₂CH₃ and —C(O)Cy².

In various aspects, R³ is selected from hydrogen, halogen, C1-C8 alkyl, and C1-C8 alkoxy. In a further aspect, R³ is selected from hydrogen, halogen, C1-C4 alkyl, and C1-C4 alkoxy. In yet a further aspect, R³ is selected from hydrogen, —F, —Cl, methyl, ethyl, n-propyl, isopropyl, —OCH₃, —OCH₂CH₃, —OCH(CH₃)₂, and —OCH₂CH₂CH₃. In a still further aspect, R³ is selected from hydrogen, —F, —Cl, methyl, ethyl, —OCH₃, and —OCH₂CH₃. In a still further aspect, R³ is selected from hydrogen, —F, —Cl, methyl, and —OCH₃.

In various aspects, R³ is C1-C8 alkoxy. In a further aspect, R³ is C1-C4 alkoxy. In a still further aspect, R³ is selected from —OCH₃, —OCH₂CH₃, —OCH(CH₃)₂, and —OCH₂CH₂CH₃. In yet a further aspect, R³ is selected from —OCH₃ and —OCH₂CH₃. In yet a further aspect, R³ is —OCH₃.

In various aspects, R³ is selected from hydrogen, halogen, C1-C8 haloalkyl, and C1-C8 haloalkoxy. In a further aspect, R³ is selected from hydrogen, halogen, C1-C4 haloalkyl, and C1-C4 haloalkoxy. In a still further aspect, R³ is selected from hydrogen, halogen, C1-C4 alkyl, C1-C4 haloalkyl, and C1-C4 haloalkoxy. In yet a further aspect, R³ is selected from hydrogen, —F, —Cl, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CH(Cl)CH₃. In a still further aspect, R³ is selected from hydrogen, —F, —Cl, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CH(Cl)CH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCH(Cl)CH₃. In yet a further aspect, R³ is selected from hydrogen, —F, —Cl, —CF₃, —CHF₂, —CH₂F, —CCl₃, —CHCl₂, —CH₂Cl, —OCF₃, —OCHF₂, —OCH₂F, —OCCl₃, —OCHCl₂, and —OCH₂Cl.

In various aspects, R³ is selected from C1-C8 haloalkyl and C1-C8 haloalkoxy. In a further aspect, R³ is selected from C1-C4 haloalkyl and C1-C4 haloalkoxy. In yet a further aspect, R³ is selected from —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, and —OCH₂CH(Cl)CH₃. In a still further aspect, R³ is selected from —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CH(Cl)CH₃, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, and —OCH(Cl)CH₃. In yet a further aspect, R³ is selected from —CF₃, —CHF₂, —CH₂F, —CCl₃, —CHCl₂, —CH₂Cl, —OCF₃, —OCHF₂, —OCH₂F, —OCCl₃, —OCHCl₂, and —OCH₂Cl.

In various aspects, R³ is selected from hydrogen and C1-C8 alkyl. In a further aspect, R³ is selected from hydrogen and C1-C4 alkyl. In yet a further aspect, R³ is selected from hydrogen, methyl, ethyl, n-propyl, and isopropyl. In a still further aspect, R³ is selected from hydrogen, methyl, and ethyl. In yet a further aspect, R³ is selected from hydrogen and ethyl. In an even further aspect, R³ is selected from hydrogen and methyl.

In various aspects, R³ is selected from hydrogen and halogen. In a further aspect, R³ is selected from hydrogen, —F, and —Cl. In a still further aspect, R³ is selected from hydrogen and —F. In yet a further aspect, R³ is selected from hydrogen and —Cl.

In various aspects, R³ is halogen. In a further aspect, R³ is selected from —F, —Cl, and —Br. In a still further aspect. R³ is selected from —F and —Cl. In yet a further aspect, R³ is —Cl. In an even further aspect, R³ is —F.

In various aspects, R³ is tert-butyl.

In various aspects, R³ is hydrogen.

g. R^{4A}, R^{4B}, R^{4C}, and R^{4D} Groups

In one aspect, each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹. In a further aspect, each of R^{4a}, R^{4b}, R^{4c}, and R^{4d} is independently selected from hydrogen, halogen, —CN, —NH₂, —OH, —NO₂, —N═C═S, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —O—(C1-C4 alkyl)-R¹³, —(OCH₂CH₂), OR¹⁴, —NHR¹⁵, —B(OR¹⁶)₂, —OCy¹, and Cy¹. In yet a further aspect, each of R^{4a}, R^{4b}, R^{4c}, and R^{4d} is independently selected from hydrogen, —F, —Cl, —CN, —NH₂, —OH, —NO₂, —N═C═S, methyl, ethyl, n-propyl, isopropyl, ethenyl, n-propenyl, isopropenyl, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, —CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —CH₂CN, —CH₂CH₂CN, —CH(CH₃)CH₂CN, —CH₂CH₂CH₂CN, —CH₂OH, —CH₂CH₂OH, —CH(CH₃)CH₂OH, —CH₂CH₂CH₂OH, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, —OCH₂CH(Cl)CH₃, —OCH₃, —OCH₂CH₃. —OCH(CH₃)₂, —OCH₂CH₂CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —CH₂NH₂, —CH₂CH₂NH₂, —CH(CH₃)CH₂NH₂, —CH₂CH₂CH₂NH₂, —OCH₂R¹³, —OCH₂CH₂R¹³, —OCH(CH₃)CH₂R¹³, —OCH₂CH₂CH₂R¹³, OCH₂CH₂OR¹⁴, —(OCH₂CH₂)₂OR¹⁴, —(OCH₂CH₂)₃OR¹⁴, —NHR¹⁵, —B(OR¹⁶)₂, —OCy¹, and Cy¹. In an even further aspect, each of R^{4a}, R^{4b}, R^{4c}, and R^{4d} is independently selected from hydrogen, —F, —Cl, —CN, —NH₂, —OH, —NO₂, —N═C═S, methyl, ethyl, ethenyl, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CH(Cl)CH₃, —CH₂CN, —CH₂CH₂CN, —CH₂OH, —CH₂CH₂OH, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —OCH₂R¹³, —OCH₂CH₂R¹³, —OCH₂CH₂OR¹⁴, —(OCH₂CH₂)₂OR¹⁴, —NHR¹⁵, —B(OR¹⁶)₂, —OCy¹, and Cy¹. In a still further aspect, each of R^{4a}, R^{4b}, R^{4c}, and R^{4d} is independently selected from hydrogen, —F, —Cl, —CN, —NH₂, —OH, —NO₂, —N═C═S, methyl, —CF₃, —CHF₂, —CH₂F, —CCl₃, —CHCl₂, —CH₂Cl, —CH₂CN. —CH₂OH, —OCF₃, —OCHF₂, —OCH₂F, —OCCl₃, —OCHCl₂, —OCH₂Cl, —OCH₃, —NHCH₃, —N(CH₃)₂, —CH₂NH₂, —OCH₂R¹³, —OCH₂CH₂OR¹⁴, —NHR¹⁵, —B(OR¹⁶)₂, —OCy¹, and Cy¹.

In one aspect, each of R^{4a}, R^{4b}, R^{4c}, and R^{4d} is independently selected from hydrogen, —NH₂, C1-C8 alkoxy, —O—(C1-C8 alkyl)-R¹³, and —NHR⁵. In a further aspect, each of R^{4a}, R^{4b}, R^{4c}, and R^{4d} is independently selected from hydrogen, —NH₂, C1-C4 alkoxy, —O—(C1-C4 alkyl)-R¹³, and —NHR⁵. In yet a further aspect, each of R^{4a}, R^{4b}, R^{4c}, and R^{4d} is independently selected from hydrogen, —NH₂, —OCH₃, —OCH₂CH₃, —OCH(CH₃)₂, —OCH₂CH₂CH₃, —OCH₂R¹³, —OCH₂CH₂R¹³, —OCH(CH₃)CH₂R¹³, —OCH₂CH₂CH₂R¹³, and —NHR¹⁵. In an even further aspect, each of R^{4a}, R^{4b}, R^{4c}, and R^{4d} is independently selected from hydrogen, —NH₂, —OCH₃, —OCH₂CH₃, —OCH₂R¹³, —OCH₂CH₂R¹³, -and —NHR¹⁵. In a still further aspect, each of R^{4a}, R^{4b}, R^{4c}, and R^{4d} is independently selected from hydrogen, —NH₂, —OCH₃, —OCH₂R¹³, and —NHR¹⁵.

In various aspects, each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, and Cy¹. In a further aspect, each of R^{4a}, R^{4b}, R^{4c}, and R^{4d} is independently selected from hydrogen, halogen, —CN, —NH₂, —OH, —NO₂, —N═C═S, C1-C4 alkyl, C2-C4 alkenyl, and Cy¹. In yet a further aspect, each of R^{4a}, R^{4b}, R^{4c}, and R^{4d} is independently selected from hydrogen, —F, —Cl, —CN, —NH₂, —OH, —NO₂, —N═C═S, methyl, ethyl, n-propyl, isopropyl, ethenyl, n-propenyl, isopropenyl, and Cy¹. In an even further aspect, each of R^{4a}, R^{4b}, R^{4c}, and R^{4d} is independently selected from hydrogen, —F, —Cl, —CN, —NH₂, —OH, —NO₂, —N═C═S, methyl, ethyl, ethenyl, and Cy¹. In a still further aspect, each of R^{4a}, R^{4b}, R^{4c}, and R^{4d} is independently selected from hydrogen, —F, —Cl, —CN, —NH₂, —OH, —NO₂, —N═C═S, methyl, and Cy¹.

In various aspects, each of R^{4a}, R^{4b}, R^{4c}, and R^{4d} is independently selected from hydrogen, halogen, —CN, —NH₂, —OH, —NO₂, —N═C═S, C1-C8 haloalkyl, C1-C8 cyanoalkyl, C1-C8 haloalkoxy, —B(OR¹⁶)₂, and Cy¹. In a further aspect, each of R^{4a}, R^{4b}, R^{4c}, and R^{4d} is independently selected from hydrogen, halogen, —CN, —NH₂, —OH, —NO₂, —N═C═S, C1-C4 haloalkyl, C1-C4 cyanoalkyl, C1-C4 haloalkoxy, —B(OR¹⁶)₂, and Cy¹. In yet a further aspect, each of R^{4a}, R^{4b}, R^{4c}, and R^{4d} is independently selected from hydrogen, —F, —Cl, —CN, —NH₂, —OH, —NO₂, —N═C═S, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —CH₂CN, —CH₂CH₂CN, —CH(CH₃)CH₂CN, —CH₂CH₂CH₂CN, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, —OCH₂CH(Cl)CH₃, —B(OR¹⁶)₂, and Cy¹. In an even further aspect, each of R^{4a}, R^{4b}, R^{4c}, and R^{4d} is independently selected from hydrogen, —F, —Cl, —CN, —NH₂, —OH, —NO₂, —N═C═S, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CH(Cl)CH₃, —CH₂CN, —CH₂CH₂CN, —OCF₃, —OCHF₂, —OCH₂F, —OCH₂CF₃, —OCH₂CHF₂, —OCH₂CH₂F, OCH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —B(OR¹⁶)₂, and Cy¹. In a still further aspect, each of R^{4a}, R^{4b}, R^{4c}, and R^{4d} is independently selected from hydrogen, —F, —Cl, —CN, —NH₂, —OH, —NO₂, —N═C═S, —CF₃, —CHF₂, —CH₂F, —CCl₃, —CHCl₂, —CH₂Cl, —CH₂CN, —OCF₃, —OCHF₂, —OCH₂F, —OCCl₃, —OCHCl₂, —OCH₂Cl, —B(OR¹⁶)₂, and Cy¹.

In various aspects, each of R^{4a}, R^{4b}, R^{4c}, and R^{4d} is independently selected from hydrogen, halogen, —CN, —NH₂, —OH, —NO₂, —N═C═S, C1-C8 hydroxyalkyl, C1-C8 alkoxy, —O—(C1-C8 alkyl)-R¹³, —(OCH₂CH₂)ₙOR¹⁴, —OCy¹, and Cy¹. In a further aspect, each of R^{4a}, R^{4b}, R^{4c}, and R^{4d} is independently selected from hydrogen, halogen, —CN, —NH₂, —OH, —NO₂, —N═C═S, C1-C4 hydroxyalkyl, C1-C4 alkoxy, —O—(C1-C4 alkyl)-R¹³, —(OCH₂CH₂), OR¹⁴, —OCy¹, and Cy¹. In yet a further aspect, each of R^{4a}, R^{4b}, R^{4c}, and R^{4d} is independently selected from hydrogen, —F, —Cl, —CN, —NH₂, —OH, —NO₂, —N═C═S, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OCH₂R¹³, —OCH₂CH₂R¹³, —OCH(CH₃)CH₂R¹³, —OCH₂CH₂CH₂R¹³, —OCH₂CH₂OR¹⁴, —(OCH₂CH₂)₂OR¹⁴, —(OCH₂CH₂)₃OR¹⁴, —OCy¹, and Cy¹. In an even further aspect, each of R^{4a}, R^{4b}, R^{4c}, and R^{4d} is independently selected from hydrogen, —F, —Cl, —CN, —NH₂, —OH, —NO₂, —N═C═S, —CH₂OH, —CH₂CH₂OH, —OCH₃, —OCH₂CH₃, —OCH₂R¹³, —OCH₂CH₂R¹³, —OCH₂CH₂OR¹⁴, —(OCH₂CH₂)₂OR¹⁴, —OCy¹, and Cy¹. In a still further aspect, each of R^{4a}, R^{4b}, R^{4c}, and R^{4d} is independently selected from hydrogen, —F, —Cl, —CN, —NH₂, —OH, —NO₂, —N═C═S, —CH₂OH, —OCH₃, —OCH₂R¹³, —OCH₂CH₂OR¹⁴, —OCy¹, and Cy¹.

In various aspects, each of R^{4a}, R^{4b}, R^{4c}, and R^{4d} is independently selected from hydrogen, halogen, —CN, —NH₂, —OH, —NO₂, —N═C═S, C1-C8 alkylamino, (C1-C8)(C1-C8) dialkylamino, C1-C8 alkylamino, —NHR¹⁵, and Cy¹. In a further aspect, each of R^{4a}, R^{4b}, R^{4c}, and R^{4d} is independently selected from hydrogen, halogen, —CN, —NH₂, —OH, —NO₂, —N═C═S, C1-C4 alkylamino, (C1-C4)(C1-C4) dialkylamino, C1-C4 aminoalkyl, —NHR¹⁵, and Cy¹. In yet a further aspect, each of R^{4a}, R^{4b}, R^{4c}, and R^{4d} is independently selected from hydrogen, —F, —Cl, —CN, —NH₂, —OH, —NO₂, —N═C═S, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —CH₂NH₂, —CH₂CH₂NH₂, —CH(CH₃)CH₂NH₂, —CH₂CH₂CH₂NH₂, —NHR¹⁵, and Cy¹. In an even further aspect, each of R^{4a}, R^{4b}, R^{4c}, and R^{4d} is independently selected from hydrogen, —F, —Cl. —CN, —NH₂, —OH, —NO₂, —N═C═S, —NHCH₃, —NHCH₂CH₃, —N(CH₃)₂, —N(CH₃)CH₂CH₃, —CH₂NH₂, —CH₂CH₂NH₂, —NHR¹⁵, and Cy¹. In a still further aspect, each of R^{4a}, R^{4b}, R^{4c}, and R^{4d} is independently selected from hydrogen, —F, —Cl, —CN, —NH₂, —OH, —NO₂, —N═C═S, —NHCH₃, —N(CH₃)₂, —CH₂NH₂, —NHR⁵, and Cy¹.

In various aspects, each of R^{4a}, R^{4b}, R^{4c}, and R^{4d} is independently selected from hydrogen and C1-C8 alkyl. In a further aspect, each of R^{4a}, R^{4b}, R^{4c}, and R^{4d} is independently selected from hydrogen and C1-C4 alkyl. In yet a further aspect, each of R^{4a}, R^{4b}, R^{4c}, and R^{4d} is independently selected from hydrogen, methyl, ethyl, n-propyl, and isopropyl. In an even further aspect, each of R^{4a}, R^{4b}, R^{4c}, and R^{4d} is independently selected from hydrogen, methyl, and ethyl. In a still further aspect, each of R^{4a}, R^{4b}, R^{4c}, and R^{4d} is independently selected from hydrogen and methyl.

In various aspects, each of R^{4a}, R^{4b}, R^{4c}, and R^{4d} is independently selected from hydrogen and halogen. In a further aspect, each of R^{4a}, R^{4b}, R^{4c}, and R^{4d} is independently selected from hydrogen, —F, —Br, and —Cl. In yet a further aspect, each of R^{4a}, R^{4b}, R^{4c}, and R^{4d} is independently selected from hydrogen, —F, and —Cl. In an even further aspect, each of R^{4a}, R^{4b}, R^{4c}, and R^{4d} is independently selected from hydrogen and —Cl. In a still further aspect, each of R^{4a}, R^{4b}, R^{4c}, and R^{4d} is independently selected from hydrogen and —Cl.

In various aspects, at least one of R^{4a}, R^{4b}, R^{4c}, and R^{4d} is hydrogen. In a still further aspect, at least two of R^{4a}, R^{4b}, R^{4c}, and R^{4d} is hydrogen. In yet a further aspect, at least three of R^{4a}, R^{4b}, R^{4c}, and R^{4d} is hydrogen. In an even further aspect, each of R^{4a}, R^{4b}, R^{4c}, and R^{4d} is hydrogen.

In various aspects, R^{4d} is selected from halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂), OR¹⁴, —NHR¹⁵, —B(OR¹⁶)₂, —OCy¹, and Cy¹. In a further aspect, R^{4d} is selected from hydrogen, halogen, —CN, —NH₂, —OH, —NO₂, —N═C═S, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —O—(C1-C4 alkyl)-R¹³, —(OCH₂CH₂)OR¹⁴, —NHR¹⁵, —B(OR¹⁶)₂, —OCy¹, and Cy¹. In yet a further aspect, R^{4d} is selected from hydrogen, —F, —Cl, —CN, —NH₂, —OH, —NO₂, —N═C═S, methyl, ethyl, n-propyl, isopropyl, ethenyl, n-propenyl, isopropenyl, —CF₃, —CHF₂, —CH₂F, —CH₂CF₃, —CH₂CHF₂, —CH₂CH₂F, —CH(F)CH₃, —CH(CH₃)CF₃, —CH(CH₃)CHF₂, —CH(CH₃)CH₂F, —CH₂CH₂CF₃, CH₂CH₂CHF₂, —CH₂CH₂CF₃, —CH₂CH(F)CH₃, —CCl₃, —CHCl₂, —CH₂Cl, —CH₂CCl₃, —CH₂CHCl₂, —CH₂CH₂Cl, —CH(Cl)CH₃, —CH(CH₃)CCl₃, —CH(CH₃)CHCl₂, —CH(CH₃)CH₂Cl, —CH₂CH₂CCl₃, —CH₂CH₂CHCl₂, —CH₂CH₂CCl₃, —CH₂CH(Cl)CH₃, —CH₂CN, —CH₂CH₂CN, —CH(CH₃)CH₂CN, —CH₂CH₂CH₂CN, —CH₂OH, —CH₂CH₂OH, —CH(CH₃)CH₂OH, —CH₂CH₂CH₂OH, —OCF₃, —OCHF₂, —OCH₂F, —OCH₂CF₃, —OCH₂CHF₂, —OCH₂CH₂F, —OCH(F)CH₃, —OCH(CH₃)CF₃, —OCH(CH₃)CHF₂, —OCH(CH₃)CH₂F, —OCH₂CH₂CF₃, —OCH₂CH₂CHF₂, —OCH₂CH₂CF₃, —OCH₂CH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH(CH₃)CCl₃, —OCH(CH₃)CHCl₂, —OCH(CH₃)CH₂Cl, —OCH₂CH₂CCl₃, —OCH₂CH₂CHCl₂, —OCH₂CH₂CCl₃, —OCH₂CH(Cl)CH₃, —OCH₃, —OCH₂CH₃, —OCH(CH₃)₂, —OCH₂CH₂CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —CH₂NH₂, —CH₂CH₂NH₂, —CH(CH₃)CH₂NH₂, —CH₂CH₂CH₂NH₂, —OCH₂R¹³, —OCH₂CH₂R¹³, —OCH(CH₃)CH₂R¹³, —OCH₂CH₂CH₂R¹³, —OCH₂CH₂OR¹⁴, —(OCH₂CH₂)₂OR¹⁴, —(OCH₂CH₂)₃OR¹⁴, —NHR¹⁵, —B(OR¹⁶)₂, —OCy¹, and Cy¹. In an even further aspect, R^{4d} is selected from hydrogen, —F, —Cl, —CN, —NH₂, —OH, —NO₂, —N═C═S, methyl, ethyl, ethenyl, —CF₃, —CHF₂, —CH₂F, —CH₂CF₃, —CH₂CHF₂, —CH₂CH₂F, —CH(F)CH₃, —CCl₃, —CHCl₂, —CH₂Cl, —CH₂CCl₃, —CH₂CHCl₂, —CH₂CH₂Cl, —CH(Cl)CH₃, —CH₂CN, —CH₂CH₂CN, —CH₂OH, —CH₂CH₂OH, —OCF₃, —OCHF₂, —OCH₂F, —OCH₂CF₃, —OCH₂CHF₂, —OCH₂CH₂F, —OCH(F)CH₃, —OCCl₃, —OCHCl₂, —OCH₂Cl, —OCH₂CCl₃, —OCH₂CHCl₂, —OCH₂CH₂Cl, —OCH(Cl)CH₃, —OCH₃, —OCH₂CH₃, —NHCH₃, NHCH₂CH₃, —N(CH₃)₂, —N(CH₃)CH₂CH₃, —CH₂NH₂, —CH₂CH₂NH₂, —OCH₂R¹³, —OCH₂CH₂R¹³, —OCH₂CH₂OR¹⁴, —(OCH₂CH₂)₂OR¹⁴, —NHR¹⁵, —B(OR¹⁶)₂, —OCy¹, and Cy¹.

In a still further aspect, R^{4d} is selected from hydrogen, —F, —Cl, —CN, —NH₂, —OH, —NO₂, —N═C═S, methyl, —CF₃, —CHF₂, —CH₂F, —CCl₃, —CHCl₂, —CH₂Cl, —CH₂CN, —CH₂OH, —OCF₃, —OCHF₂, —OCH₂F, —OCCl₃, —OCHCl₂, —OCH₂Cl, —OCH₃, —NHCH₃, —N(CH₃)₂, —CH₂NH₂, —OCH₂R¹³, —OCH₂CH₂OR¹⁴, —NHR¹⁵, —B(OR¹⁶)₂, —OCy¹, and Cy¹.

In various aspects, R^{4d} is selected from C1-C8 alkoxy, C1-C8 alkylamino, (C1-C8)(C1-C8) dialkylamino, —O—(C1-C8 alkyl)-R¹³, —(OCH₂CH₂)ₙOR¹⁴, and —NHR¹⁵. In a further aspect, R^{4d} is selected from C1-C4 alkoxy, C1-C4 alkylamino, (C1-C4)(C1-C4) dialkylamino, —O—(C1-C4 alkyl)-R¹³, —(OCH₂CH₂)ₙOR¹⁴, and —NHR¹⁵. In yet a further aspect, R^{4d} is selected from —OCH₃, —OCH₂CH₃, —OCH(CH₃)₂, —OCH₂CH₂CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —OCH₂R¹³, —OCH₂CH₂R¹³, OCH(CH₃)CH₂R¹³, —OCH₂CH₂CH₂R¹³, —OCH₂CH₂OR¹⁴, —(OCH₂CH₂)₂OR¹⁴, —(OCH₂CH₂)₃OR¹⁴, and —NHR¹⁵. In an even further aspect, R^{4d} is selected from —OCH₃, —OCH₂CH₃, —NHCH₃, —NHCH₂CH₃, —N(CH₃)₂, —N(CH₃)CH₂CH₃, —OCH₂R¹³, —OCH₂CH₂R¹³, —OCH₂CH₂OR¹⁴, —(OCH₂CH₂)₂OR¹⁴, and —NHR¹⁵. In a still further aspect, R^{4d} is selected from —OCH₃, —NHCH₃, —N(CH₃)₂, —OCH₂R¹³, —OCH₂CH₂OR¹⁴, and —NHR¹⁵.

In various aspects, R^{4d} is selected from C1-C8 alkoxy and —O—(C1-C8 alkyl)-R¹³. In a further aspect, R^{4d} is selected from C1-C4 alkoxy and —O—(C1-C4 alkyl)-R¹³. In yet a further aspect, R^{4d} is selected from —OCH₃, —OCH₂CH₃, —OCH(CH₃)₂, —OCH₂CH₂CH₃, —OCH₂R¹³, —OCH₂CH₂R¹³, —OCH(CH₃)CH₂R¹³, and —OCH₂CH₂CH₂R¹³. In an even further aspect, R^{4d} is selected from —OCH₃, —OCH₂CH₃, —OCH₂R¹³, and —OCH₂CH₂R¹³. In a still further aspect, R^{4d} is selected from —OCH₃ and —OCH₂R¹³.

In various aspects, R^{4d} is selected from hydrogen, C1-C8 alkoxy, —O—(C1-C8 alkyl)-R¹³, and —(OCH₂CH₂)ₙOR¹⁴. In a further aspect, R^{4d} is selected from hydrogen, C1-C4 alkoxy, —O—(C1-C4 alkyl)-R¹³, and —(OCH₂CH₂)ₙOR¹⁴. In yet a further aspect, R^{4d} is selected from hydrogen, —OCH₃, —OCH₂CH₃, —OCH(CH₃)₂, —OCH₂CH₂CH₃, —OCH₂R¹³, —OCH₂CH₂R¹³, —OCH(CH₃)CH₂R¹³, —OCH₂CH₂CH₂R¹³, —OCH₂CH₂OR¹⁴, —(OCH₂CH₂)₂OR¹⁴, and —(OCH₂CH₂)₃OR¹⁴. In an even further aspect, R^{4d} is selected from hydrogen, —OCH₃, —OCH₂CH₃, —OCH₂R¹³, —OCH₂CH₂R¹³, —OCH₂CH₂OR¹⁴, and —(OCH₂CH₂)₂OR¹⁴. In a still further aspect, R^{4d} is selected from hydrogen, —OCH₃, —OCH₂R¹³, and —OCH₂CH₂OR¹⁴.

In various aspects, R^{4d} is selected from —NH₂, —N═C═S, C1-C8 alkoxy, C1-C8 alkylamino, (C1-C8)(C1-C8) dialkylamino, —O—(C1-C8 alkyl)-R¹³, —(OCH₂CH₂)OR¹⁴, —NHR¹⁵, and Cy¹. In a further aspect, R^{4d} is selected from —NH₂, —N═C═S, C1-C4 alkoxy, C1-C4 alkylamino, (C1-C4)(C1-C4) dialkylamino, —O—(C1-C4 alkyl)-R¹³, —(OCH₂CH₂)ₙOR¹⁴, —NHR¹⁵, and Cy¹. In yet a further aspect, R^{4d} is selected from —NH₂, —N═C═S, —OCH₃, —OCH₂CH₃, —OCH(CH₃)₂, —OCH₂CH₂CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —OCH₂R¹³, —OCH₂CH₂R¹³, —OCH(CH₃)CH₂R¹³, —OCH₂CH₂CH₂R¹³, —OCH₂CH₂OR¹⁴, —(OCH₂CH₂)₂OR¹⁴, —(OCH₂CH₂)₃OR¹⁴, —NHR¹⁵, and Cy¹. In an even further aspect, R^{4d} is selected from —NH₂, —N═C═S, —OCH₃, —OCH₂CH₃, —NHCH₃, —NHCH₂CH₃, —N(CH₃)₂, —N(CH₃)CH₂CH₃, —OCH₂R¹³, —OCH₂CH₂R¹³, —OCH₂CH₂OR¹⁴, —(OCH₂CH₂)₂OR¹⁴, —NHR¹⁵, and Cy¹. In a still further aspect, R^{4d} is selected from hydrogen, —NH₂, —N═C═S, —OCH₃, —NHCH₃, —N(CH₃)₂, —OCH₂R¹³, —OCH₂CH₂OR¹⁴, —NHR¹⁵, and Cy¹ .

In various aspects, R^{4d} is C1-C8 alkoxy. In a further aspect, R^{4d} is C1-C4 alkoxy. In yet a further aspect, R^{4d} is selected from —OCH₃, —OCH₂CH₃, —OCH(CH₃)₂, and —OCH₂CH₂CH₃. In an even further aspect, R^{4d} is selected from —OCH₃ and —OCH₂CH₃. In a still further aspect, R^{4d} is —OCH₃.

In various aspects, R^{4b} is selected from hydrogen, —NH₂, and —NHR¹⁵. In a further aspect, R^{4b} is selected from hydrogen and —NH₂. In a still further aspect, R^{4b} is selected from hydrogen and —NHR¹⁵. In yet a further aspect, R^{4b} is —NH₂. In an even further aspect, R^{4b} is —NHR¹⁵.

In various aspects, each of R^{4b} and R^{4d} is hydrogen. In a still further aspect, each of R^{4b} and R^{4c} is hydrogen. In yet a further aspect, In various aspects, each of R^{4a}, R^{4b}, and R^{4c} is hydrogen.

h. R⁵ Groups

In one aspect, R⁵ is selected from hydrogen, halogen, and C1-C4 alkoxy. In a further aspect, R⁵ is selected from hydrogen, —F, —Cl, —OCH₃, —OCH₂CH₃, —OCH(CH₃)₂, and —OCH₂CH₂CH₃.

In various aspects, R⁵ is selected from hydrogen and C1-C4 alkoxy. In a further aspect, R⁵ is selected from hydrogen, —OCH₃, —OCH₂CH₃, —OCH(CH₃)₂, and —OCH₂CH₂CH₃. In a still further aspect, R⁵ is selected from hydrogen, —OCH₃, and —OCH₂CH₃. In yet a further aspect, R⁵ is selected from hydrogen and —OCH₃. In a still further aspect, R⁵ is —OCH₃.

In various aspects, R⁵ is C1-C4 alkoxy. In a further aspect, R⁵ is selected from OCH₃, —OCH₂CH₃, —OCH(CH₃)₂, and —OCH₂CH₂CH₃. In a still further aspect, R⁵ is selected from —OCH₃ and —OCH₂CH₃.

In various aspects, R⁵ is selected from hydrogen and halogen. In a further aspect, R⁵ is selected from hydrogen, —F, and —Cl. In yet a further aspect, R⁵ is selected from hydrogen and —F. In a still further aspect, R⁵ is selected from hydrogen and —C1.

In various aspects, R⁵ is halogen. In a further aspect, R⁵ is selected from —F, —Cl, and —Br. In a still further aspect, R⁵ is selected from —F and —Cl. In yet a further aspect, R⁵ is —Cl. In an even further aspect, R⁵ is —F.

In various aspects, R⁵ is hydrogen.

i. R⁶ Groups

In one aspect, R⁶ is selected from hydrogen and C1-C4 alkoxy. In a further aspect, R⁶ is selected from hydrogen, —OCH₃, —OCH₂CH₃, —OCH(CH₃)₂, and —OCH₂CH₂CH₃. In a still further aspect, R⁶ is selected from hydrogen, —OCH₃, and —OCH₂CH₃. In yet a further aspect, R⁶ is selected from hydrogen and —OCH₃. In a still further aspect, R⁶ is —OCH₃.

In various aspects, R⁶ is C1-C4 alkoxy. In a further aspect, R⁶ is selected from hydrogen, —OCH₃, —OCH₂CH₃, —OCH(CH₃)₂, and —OCH₂CH₂CH₃. In a still further aspect, R⁶ is selected from hydrogen, —OCH₃, and —OCH₂CH₃. In yet a further aspect, R⁶ is selected from hydrogen and —OCH₃. In a still further aspect, R⁶ is —OCH₃.

In various aspects, R⁶ is hydrogen.

j. R⁷ Groups

In one aspect, R⁷ is selected from hydrogen and C1-C4 alkyl. In a further aspect, R⁷ is selected from hydrogen, methyl, ethyl, n-propyl, and isopropyl. In yet a further aspect, R⁷ is selected from hydrogen, methyl, and ethyl. In a still further aspect, R⁷ is selected from hydrogen and methyl. In yet a further aspect, R⁷ is methyl.

In various aspects, R⁷ is C1-C4 alkyl. In a further aspect, R⁷ is selected from methyl, ethyl, n-propyl, and isopropyl. In yet a further aspect, R⁷ is selected from methyl and ethyl.

In various aspects, R⁷ is hydrogen.

k. R⁸ Groups

In one aspect, R⁸ is selected from hydrogen, halogen, and C1-C4 alkoxy. In a further aspect, R⁸ is selected from hydrogen, —F, —Cl, —OCH₃, —OCH₂CH₃, —OCH(CH₃)₂, and —OCH₂CH₂CH₃. In a still further aspect, R⁸ is selected from hydrogen, —F, —Cl, —OCH₃, and —OCH₂CH₃. In yet a further aspect, R⁸ is selected from hydrogen, —F, —Cl, and —OCH₃.

In one aspect. R⁸ is selected from hydrogen and halogen. In a further aspect, R⁸ is selected from hydrogen, —F, —Cl, and —Br. In a still further aspect, R⁸ is selected from hydrogen, —F, and —Cl. In yet a further aspect, R⁸ is selected from hydrogen and —F. In an even further aspect, R⁸ is selected from hydrogen and —Cl.

In one aspect, R⁸ is selected from hydrogen and C1-C4 alkoxy. In a further aspect, R⁸ is selected from hydrogen, —OCH₃, —OCH₂CH₃, —OCH(CH₃)₂, and —OCH₂CH₂CH₃. In a still further aspect, R⁸ is selected from hydrogen, —OCH₃, and —OCH₂CH₃. In yet a further aspect, R⁸ is selected from hydrogen and —OCH₃.

In various aspects, R⁸ is halogen. In a further aspect, R⁸ is selected from —F, —Cl, and —Br. In a still further aspect, R⁸ is selected from —F and —Cl. In yet a further aspect, R⁸ is —Cl. In an even further aspect, R⁸ is —F.

In various aspects, R⁸ is C1-C4 alkoxy. In a further aspect, R⁸ is selected from —OCH₃, —OCH₂CH₃, —OCH(CH₃)₂, and —OCH₂CH₂CH₃. In a still further aspect, R⁸ is selected from —OCH₃ and —OCH₂CH₃. In yet a further aspect, R is —OCH₃.

In various aspects, R⁸ is hydrogen.

### 1. R¹⁰ Groups

In one aspect, R¹⁰ is selected from hydrogen and C1-C4 alkyl. In a further aspect, R¹⁰ is selected from hydrogen, methyl, ethyl, n-propyl, and isopropyl. In yet a further aspect, R¹⁰ is selected from hydrogen, methyl, and ethyl. In a still further aspect, R¹⁰ is selected from hydrogen and methyl. In yet a further aspect, R¹⁰ is methyl.

In various aspects, R¹⁰ is C1-C4 alkyl. In a further aspect, R¹⁰ is selected from methyl, ethyl, n-propyl, and isopropyl. In yet a further aspect, R¹⁰ is selected from methyl and ethyl.

In various aspects, R¹⁰ is hydrogen.

m. R¹¹ Groups

In one aspect, R¹¹, when present, is selected from hydrogen and C1-C4 alkyl. In a further aspect, R¹¹, when present, is selected from hydrogen, methyl, ethyl, n-propyl, and isopropyl. In yet a further aspect, R¹¹, when present, is selected from hydrogen, methyl, and ethyl. In a still further aspect, R¹¹, when present, is selected from hydrogen and methyl. In yet a further aspect, R¹¹ is methyl.

In various aspects, R¹¹, when present, is C1-C4 alkyl. In a further aspect, R¹¹, when present, is selected from methyl, ethyl, n-propyl, and isopropyl. In yet a further aspect, R¹¹, when present, is selected from methyl and ethyl.

In various aspects, R¹¹, when present, is hydrogen.

n. R¹² Groups

In one aspect, R¹² is selected from hydrogen and C1-C4 alkoxy. In a further aspect, R¹² is selected from hydrogen, —OCH₃, —OCH₂CH₃, —OCH(CH₃)₂, and —OCH₂CH₂CH₃. In a still further aspect, R¹² is selected from hydrogen, —OCH₃, and —OCH₂CH₃. In yet a further aspect, R¹² is selected from hydrogen and —OCH₃.

In various aspects, R¹² is C1-C4 alkoxy. In a further aspect, R¹² is selected from —OCH₃, —OCH₂CH₃, —OCH(CH₃)₂, and —OCH₂CH₂CH₃. In a still further aspect, R¹² is selected from —OCH₃ and —OCH₂CH₃. In yet a further aspect, R¹² is —OCH₃.

In various aspects, R¹² is hydrogen.

o. R¹³ Groups

In one aspect, R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

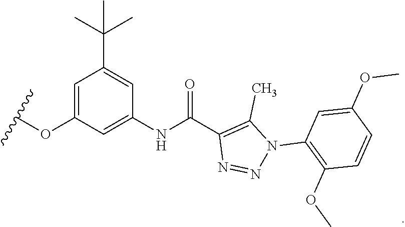 [SMILES: *Oc1cc(NC(=O)c2nnn(-c3cc(OC)ccc3OC)c2C)cc(C(C)(C)C)c1]

In a further aspect, R¹³, when present, is selected from —F, —Cl, —CN, —NH₂, —OH, —C≡CH, —CHO, —CO₂H, —CO₂CH₃, —CO₂CH₂CH₃, —CO₂CH(CH₃)₂, —CO₂CH₂CH₂CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, unsubstituted morpholine, and a structure represented by a formula:

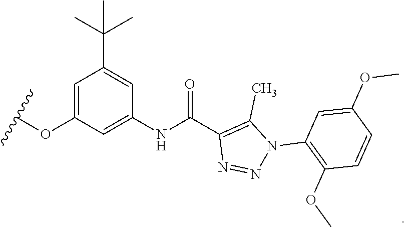 [SMILES: *Oc1cc(NC(=O)c2nnn(-c3cc(OC)ccc3OC)c2C)cc(C(C)(C)C)c1]

In a still further aspect, R¹³, when present, is selected from —F, —Cl, —CN, —NH₂, —OH, —C≡CH, —CHO, —CO₂H, —CO₂CH₃, —CO₂CH₂CH₃. —NHCH₃, —NHCH₂CH₃, —N(CH₃)₂, —N(CH₃)CH₂CH₃, unsubstituted morpholine, and a structure represented by a formula:

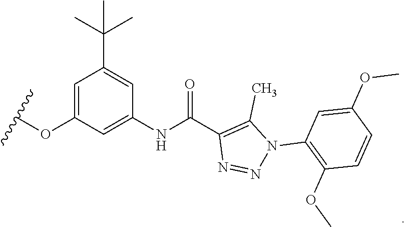 [SMILES: *Oc1cc(NC(=O)c2nnn(-c3cc(OC)ccc3OC)c2C)cc(C(C)(C)C)c1]

In yet a further aspect, R¹³, when present, is selected from —F, —Cl, —CN, —NH₂, —OH, —C≡CH, —CHO, —CO₂H, —CO₂CH₃, —NHCH₃, —N(CH₃)₂, unsubstituted morpholine, and a structure represented by a formula:

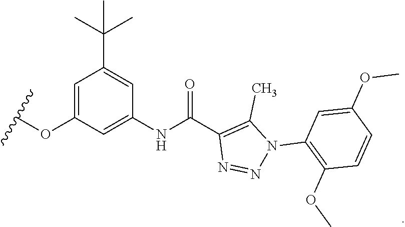 [SMILES: *Oc1cc(NC(=O)c2nnn(-c3cc(OC)ccc3OC)c2C)cc(C(C)(C)C)c1]

In one aspect, R¹³, when present, is selected from —CO₂H and —CO₂(C1-C4 alkyl). In a further aspect, R¹³, when present, is selected from —CO₂H, —CO₂CH₃, —CO₂CH₂CH₃, —CO₂CH(CH₃)₂, and —CO₂CH₂CH₂CH₃. In a still further aspect, R¹³, when present, is selected from —CO₂H, —CO₂CH₃, and —CO₂CH₂CH₃. In yet a further aspect, R¹³, when present, is selected from —CO₂H and —CO₂CH₃.

In various aspects, R¹³, when present, is selected from halogen, —CN, —NH₂, —OH, —C≡CH, —CHO, —CO₂H, —CO₂(C1-C4 alkyl), C1-C4 alkylamino, and (C1-C4)(C1-C4) dialkylamino. In a further aspect, R¹³, when present, is selected from —F, —Cl, —CN, —NH₂, —OH, —C≡CH, —CHO, —CO₂H, —CO₂CH₃, —CO₂CH₂CH₃, —CO₂CH(CH₃)₂, —CO₂CH₂CH₂CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, and —N(CH₃)CH₂CH₂CH₃. In a still further aspect, R¹³, when present, is selected from —F, —Cl, —CN, —NH₂, —OH, —C≡CH, —CHO, —CO₂H, —CO₂CH₃, —CO₂CH₂CH₃, —NHCH₃, —NHCH₂CH₃, —N(CH₃)₂, and —N(CH₃)CH₂CH₃. In yet a further aspect, R¹³, when present, is selected from —F, —Cl, —CN, —NH₂, —OH, —C≡CH, —CHO, —CO₂H, —CO₂CH₃, —NHCH₃, and —N(CH₃)₂.

In various aspects, R¹³, when present, is selected from halogen, —CN, —NH₂, C1-C4 alkylamino, and (C1-C4)(C1-C4) dialkylamino. In a further aspect, R¹³, when present, is selected from —F, —Cl, —CN, —NH₂, —NHCH₃, —NHCH₂CH₃, —NHCH(CH₃)₂, NHCH₂CH₂CH₃, —N(CH₃)₂, —N(CH₃)CH₂CH₃, —N(CH₂CH₃)CH(CH₃)₂, and —N(CH₃)CH₂CH₂CH₃. In a still further aspect, R¹³, when present, is selected from —F, —Cl, —CN, —NH₂, —NHCH₃, —NHCH₂CH₃, —N(CH₃)₂, and —N(CH₃)CH₂CH₃. In yet a further aspect, R¹³, when present, is selected from —F, —Cl, —CN, —NH₂, —NHCH₃, and —N(CH₃)₂.

In various aspects, R¹³, when present, is selected from —OH, —C≡CH, —CHO, —CO₂H, and —CO₂(C1-C4 alkyl). In a further aspect, R¹³, when present, is selected from —OH, —C≡CH, —CHO, —CO₂H, —CO₂CH₃, —CO₂CH₂CH₃, —CO₂CH(CH₃)₂, and —CO₂CH₂CH₂CH₃. In a still further aspect, R¹³, when present, is selected from —OH, —C≡CH, —CHO, —CO₂H, —CO₂CH₃, and —CO₂CH₂CH₃. In yet a further aspect, R¹³, when present, is selected from —OH, —C≡CH, —CHO, —CO₂H, and —CO₂CH₃.

In various aspects, R¹³, when present, is selected from —CO₂H and —CO₂(C1-C4 alkyl). In a further aspect, R¹³, when present, is selected from —CO₂H, —CO₂CH₃, —CO₂CH₂CH₃, —CO₂CH(CH₃)₂, and —CO₂CH₂CH₂CH₃. In a still further aspect, R¹³, when present, is selected from —CO₂H, —CO₂CH₃, and —CO₂CH₂CH₃. In yet a further aspect, R¹³, when present, is selected from —CO₂H and —CO₂CH₃.

In various aspects, R¹³, when present, is selected from unsubstituted morpholine, and a structure represented by a formula:

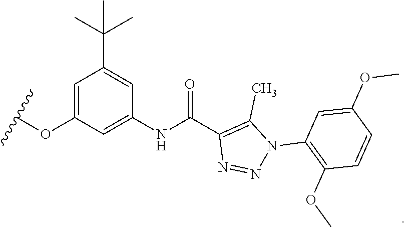 [SMILES: *Oc1cc(NC(=O)c2nnn(-c3cc(OC)ccc3OC)c2C)cc(C(C)(C)C)c1]

In a further aspect, R¹³, when present, is unsubstituted morpholine. In a still further aspect, R¹³, when present, is a structure represented by a formula:

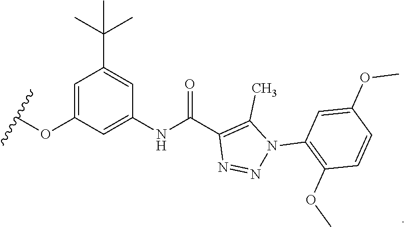 [SMILES: *Oc1cc(NC(=O)c2nnn(-c3cc(OC)ccc3OC)c2C)cc(C(C)(C)C)c1]

p. R¹⁴ Groups

In one aspect, R¹⁴, when present, is selected from hydrogen and C1-C4 alkyl. In a further aspect, R¹⁴, when present, is selected from hydrogen, methyl, ethyl, n-propyl, and isopropyl. In yet a further aspect, R¹⁴, when present, is selected from hydrogen, methyl, and ethyl. In a still further aspect, R¹⁴, when present, is selected from hydrogen and ethyl. In yet a further aspect, R¹⁴, when present, is selected from hydrogen and methyl.

In various aspects, R¹⁴, when present, is C1-C4 alkyl. In a further aspect, R¹⁴, when present, is selected from methyl, ethyl, n-propyl, and isopropyl. In yet a further aspect, R¹⁴, when present, is selected from methyl and ethyl. In an even further aspect, R¹⁴, when present, is ethyl. In a still further aspect, R¹⁴, when present, is methyl.

In various aspects, R¹⁴, when present, is hydrogen.

q. R¹⁵ Groups

In one aspect, R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl). In a further aspect, R¹¹, when present, is selected from —CH₂CO₂H, —CH₂CH₂CO₂H, —CH(CH₃)CH₂CO₂H, —CH₂CH₂CH₂CO₂H, —CH₂CO₂CH₃, —CH₂CH₂CO₂CH₃, —CH(CH₃)CH₂CO₂CH₂CH₃, —CH₂CH₂CH₂CO₂CH₃, —C(O)CH₃, —C(O)CH₂CH₃, —C(O)CH(CH₃)₂, —C(O)CH₂CH₂CH₃, —CO₂CH₃, —CO₂CH₂CH₃, —CO₂CH(CH₃)₂, —CO₂CH₂CH₂CH₃, —C(O)CH₂CO₂H, —C(O)CH₂CH₂CO₂H, —C(O)CH(CH₃)CH₂CO₂H, —C(O)CH₂CH₂CH₂CO₂H, —C(O)CH₂CO₂CH₃, —C(O)CH₂CH₂CO₂CH₂CH₂, —C(O)CH(CH₃)CH₂CO₂CH₃, and C(O)CH₂CH₂CH₂CO₂CH₃. In a still further aspect, R¹⁵, when present, is selected from —CH₂CO₂H, —CH₂CH₂CO₂H, —CH₂CO₂CH₃, —CH₂CH₂CO₂CH₃, —C(O)CH₃, —C(O)CH₂CH₃, —CO₂CH₃, —CO₂CH₂CH₃, —C(O)CH₂CO₂H, —C(O)CH₂CH₂CO₂H, —C(O)CH₂CO₂CH₃, and —C(O)CH₂CH₂CO₂CH₂CH₂. In yet a further aspect, R¹⁵, when present, is selected from —CH₂CO₂H, —CH₂CO₂CH₃, —C(O)CH₃, —CO₂CH₃, —C(O)CH₂CO₂H, and —C(O)CH₂CO₂CH₃.

In one aspect, R¹⁵, when present, is selected from —C(O)(C1-C4 alkyl) and —CO₂(C1-C4 alkyl). In a further aspect, R¹¹, when present, is selected from —C(O)CH₃, —C(O)CH₂CH₃, —C(O)CH(CH₃)₂, —C(O)CH₂CH₂CH₃, —CO₂CH₃, —CO₂CH₂CH₃, —CO₂CH(CH₃)₂, and —CO₂CH₂CH₂CH₃. In a still further aspect, R¹¹, when present, is selected from —C(O)CH₃, —C(O)CH₂CH₃, —CO₂CH₃, and —CO₂CH₂CH₃. In yet a further aspect, R¹⁵, when present, is selected from —C(O)CH₃ and —CO₂CH₃.

In various aspects, R¹⁵, when present, is selected from —(C1-C4 alkyl)CO₂H, —(C1-C4 alkyl)CO₂(C1-C4 alkyl), and —CO₂(C1-C4 alkyl). In a further aspect, R¹⁵, when present, is selected from —CH₂CO₂H, —CH₂CH₂CO₂H, —CH(CH₃)CH₂CO₂H, —CH₂CH₂CH₂CO₂H, —CH₂CO₂CH₃, —CH₂CH₂CO₂CH₃, —CH(CH₃)CH₂CO₂CH₂CH₃, —CH₂CH₂CH₂CO₂CH₃, —CO₂CH₃, —CO₂CH₂CH₃, —CO₂CH(CH₃)₂, and —CO₂CH₂CH₂CH₃. In a still further aspect, R¹⁵, when present, is selected from —CH₂CO₂H, —CH₂CH₂CO₂H, —CH₂CO₂CH₃, —CH₂CH₂CO₂CH₃, —CO₂CH₃, and —CO₂CH₂CH₃. In yet a further aspect, R¹⁵, when present, is selected from —CH₂CO₂H, —CH₂CO₂CH₃, and —CO₂CH₃.

In various aspects, R¹⁵, when present, is selected from —C(O)(C1-C4 alkyl), —C(O)(C1-C4 alkyl)CO₂H, and —C(O)(C1-C4 alkyl)CO₂(C1-C4 alkyl). In a further aspect, R¹⁵, when present, is selected from —C(O)CH₃, —C(O)CH₂CH₃, —C(O)CH(CH₃)₂, —C(O)CH₂CH₂CH₃, —C(O)CH₂CO₂H, —C(O)CH₂CH₂CO₂H, —C(O)CH(CH₃)CH₂CO₂H, —C(O)CH₂CH₂CH₂CO₂H, —C(O)CH₂CO₂CH₃, —C(O)CH₂CH₂CO₂CH₂CH₂, —C(O)CH(CH₃)CH₂CO₂CH₃, and —C(O)CH₂CH₂CH₂CO₂CH₃. In a still further aspect, R¹⁵, when present, is selected from —C(O)CH₃, —C(O)CH₂CH₃, —C(O)CH₂CO₂H, —C(O)CH₂CH₂CO₂H, —C(O)CH₂CO₂CH₃, and —C(O)CH₂CH₂CO₂CH₂CH₂. In yet a further aspect, R¹⁵, when present, is selected from —C(O)CH₃, —C(O)CH₂CO₂H, and —C(O)CH₂CO₂CH₃.

In various aspects, R¹⁵, when present, is selected from —C(O)(C1-C4 alkyl) and —CO₂(C1-C4 alkyl). In a further aspect, R¹⁵, when present, is selected from —C(O)CH₃, —C(O)CH₂CH₃, —C(O)CH(CH₃)₂, —C(O)CH₂CH₂CH₃, —CO₂CH₃, —CO₂CH₂CH₃, —CO₂CH(CH₃)₂, and —CO₂CH₂CH₂CH₃. In a still further aspect, R¹⁵, when present, is selected from —C(O)CH₃, —C(O)CH₂CH₃, —CO₂CH₃, and —CO₂CH₂CH₃. In yet a further aspect, R¹⁵, when present, is selected from —C(O)CH₃ and —CO₂CH₃.

r. R¹⁶ Groups

In one aspect, each occurrence of R¹⁶, when present, is independently selected from hydrogen and C1-C4 alkyl, or each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups.

In various aspects, each occurrence of R¹⁶, when present, is independently selected from hydrogen and C1-C4 alkyl. In a further aspect, each occurrence of R¹⁶, when present, is independently selected from hydrogen, methyl, ethyl, n-propyl, and isopropyl. In a still further aspect, each occurrence of R¹⁶, when present, is independently selected from hydrogen, methyl, and ethyl. In yet a further aspect, each occurrence of R¹⁶, when present, is independently selected from hydrogen and ethyl. In an even further aspect, each occurrence of R¹⁶, when present, is independently selected from hydrogen and methyl.

In various aspects, each occurrence of R¹⁶, when present, is independently C1—C4 alkyl. In a further aspect, each occurrence of R¹⁶, when present, is independently selected from methyl, ethyl, n-propyl, and isopropyl. In a still further aspect, each occurrence of R¹⁶, when present, is independently selected from methyl and ethyl. In yet a further aspect, each occurrence of R¹⁶, when present, is ethyl. In an even further aspect, each occurrence of R¹⁶, when present, is methyl.

In various aspects, each occurrence of R¹⁶, when present, is hydrogen.

In various aspects, each occurrence of R¹⁶, when present, is covalently bonded and, together with the intermediate atoms, comprise a 5- or 6-membered heterocycloalkyl substituted with 0, 1, 2, 3, or 4 independently selected C1-C4 alkyl groups. In a further aspect, each occurrence of R¹⁶, when present, is covalently bonded and, together with the intermediate atoms, comprise a 5- or 6-membered heterocycloalkyl substituted with 0, 1, 2, or 3 independently selected C1-C4 alkyl groups. In a still further aspect, each occurrence of R¹⁶, when present, is covalently bonded and, together with the intermediate atoms, comprise a 5- or 6-membered heterocycloalkyl substituted with 0, 1, or 2 independently selected C1-C4 alkyl groups. In yet a further aspect, each occurrence of R¹⁶, when present, is covalently bonded and, together with the intermediate atoms, comprise a 5- or 6-membered heterocycloalkyl substituted with 0 or 1 C1-C4 alkyl groups. In an even further aspect, each occurrence of R¹⁶, when present, is covalently bonded and, together with the intermediate atoms, comprise a 5- or 6-membered heterocycloalkyl monosubstituted with a C1-C4 alkyl group. In a still further aspect, each occurrence of R¹⁶, when present, is covalently bonded and, together with the intermediate atoms, comprise an unsubstituted 5- or 6-membered heterocycloalkyl.

In various aspects, each occurrence of R¹⁶, when present, is covalently bonded and, together with the intermediate atoms, comprise a 5-membered heterocycloalkyl substituted with 0, 1, 2, 3, or 4 independently selected C1-C4 alkyl groups. In a further aspect, each occurrence of R¹⁶, when present, is covalently bonded and, together with the intermediate atoms, comprise a 5-membered heterocycloalkyl substituted with 0, 1, 2, or 3 independently selected C1-C4 alkyl groups. In a still further aspect, each occurrence of R¹⁶, when present, is covalently bonded and, together with the intermediate atoms, comprise a 5-membered heterocycloalkyl substituted with 0, 1, or 2 independently selected C1-C4 alkyl groups. In yet a further aspect, each occurrence of R¹⁶, when present, is covalently bonded and, together with the intermediate atoms, comprise a 5-membered heterocycloalkyl substituted with 0 or 1 C1-C4 alkyl groups. In an even further aspect, each occurrence of R¹⁶, when present, is covalently bonded and, together with the intermediate atoms, comprise a 5-membered heterocycloalkyl monosubstituted with a C1-C4 alkyl group. In a still further aspect, each occurrence of R¹⁶, when present, is covalently bonded and, together with the intermediate atoms, comprise an unsubstituted 5-membered heterocycloalkyl.

In various aspects, each occurrence of R¹⁶, when present, is covalently bonded and, together with the intermediate atoms, comprise a 6-membered heterocycloalkyl substituted with 0, 1, 2, 3, or 4 independently selected C1-C4 alkyl groups. In a further aspect, each occurrence of R¹⁶, when present, is covalently bonded and, together with the intermediate atoms, comprise a 6-membered heterocycloalkyl substituted with 0, 1, 2, or 3 independently selected C1-C4 alkyl groups. In a still further aspect, each occurrence of R¹, when present, is covalently bonded and, together with the intermediate atoms, comprise a 6-membered heterocycloalkyl substituted with 0, 1, or 2 independently selected C1-C4 alkyl groups. In yet a further aspect, each occurrence of R¹⁶, when present, is covalently bonded and, together with the intermediate atoms, comprise a 6-membered heterocycloalkyl substituted with 0 or 1 C1-C4 alkyl groups. In an even further aspect, each occurrence of R¹⁶, when present, is covalently bonded and, together with the intermediate atoms, comprise a 6-membered heterocycloalkyl monosubstituted with a C1-C4 alkyl group. In a still further aspect, each occurrence of R¹⁶, when present, is covalently bonded and, together with the intermediate atoms, comprise an unsubstituted 6-membered heterocycloalkyl.

s. CY¹ Groups

In one aspect, Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl). In a further aspect, Cy¹, when present, is selected from a C2-C5 heterocycloalkyl and a C2-C5 heteroaryl, and is substituted with 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl). In a still further aspect, Cy¹, when present, is selected from a C2-C5 heterocycloalkyl and a C2-C5 heteroaryl, and is substituted with 1 or 2 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl). In yet a further aspect, Cy¹, when present, is selected from a C2-C5 heterocycloalkyl and a C2-C5 heteroaryl, and is monosubstituted with a group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl).

In various aspects, Cy¹, when present, is a C2-C5 heterocycloalkyl substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl). Examples of C2-C5 heterocycloalkyls include, but are not limited to, thiirane, oxirane, aziridine, thietane, azetidine, oxetane, pyrrolidine, imidazolidine, tetrahydrothiophene, tetrahydrofuran, piperidine, piperazine, thiane, and morpholine. In a further aspect, Cy¹, when present, is a C2-C5 heterocycloalkyl substituted with 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl). In a still further aspect, Cy¹, when present, is a C2-C5 heterocycloalkyl substituted with 1 or 2 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl). In yet a further aspect, Cy¹, when present, is a C2-C5 heterocycloalkyl monosubstituted with a group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl).

In various aspects, Cy¹, when present, is a C2-C5 heteroaryl substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl). Examples of C2-C5 heteroaryls include, but are not limited to, furan, pyrrole, thiophene, oxazole, isothiazole, pyridine, and triazine. In a further aspect, Cy¹, when present, is a C2-C5 heteroaryl substituted with 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl). In a still further aspect, Cy¹, when present, is a C2-C5 heteroaryl substituted with 1 or 2 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl). In yet a further aspect, Cy¹, when present, is a C2-C5 heteroaryl monosubstituted with a group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl).

t. CY² Groups

In one aspect. Cy², when present, is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a further aspect, Cy², when present, is selected from a C2-C5 heterocycloalkyl, a C6 aryl, and a C2-C5 heteroaryl, and is substituted with 0, 1, or 2 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a still further aspect, Cy², when present, is selected from a C2-C5 heterocycloalkyl, a C6 aryl, and a C2-C5 heteroaryl, and is substituted with 0 or 1 group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In yet a further aspect, Cy², when present, is selected from a C2-C5 heterocycloalkyl, a C6 aryl, and a C2-C5 heteroaryl, and is monosubstituted with a group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In an even further aspect, Cy², when present, is selected from a C2-C5 heterocycloalkyl, a C6 aryl, and a C2-C5 heteroaryl, and is unsubstituted.

In various aspects, Cy², when present, is a C2-C5 heterocycloalkyl substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. Examples of C2-C5 heterocycloalkyls include, but are not limited to, thiirane, oxirane, aziridine, thietane, azetidine, oxetane, pyrrolidine, imidazolidine, tetrahydrothiophene, tetrahydrofuran, piperidine, piperazine, thiane, and morpholine. In a further aspect, Cy², when present, is a C2-C5 heterocycloalkyl substituted with 0, 1, or 2 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a still further aspect, Cy², when present, is a C2-C5 heterocycloalkyl substituted with 0 or 1 group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In yet a further aspect, Cy², when present, is a C2-C5 heterocycloalkyl monosubstituted with a group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In an even further aspect, Cy², when present, is an unsubstituted C₂-C5 heterocycloalkyl.

In various aspects, Cy², when present, is a piperazine substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a further aspect, Cy², when present, is a piperazine substituted with 0, 1, or 2 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a still further aspect, Cy², when present, is a piperazine substituted with 0 or 1 group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In yet a further aspect, Cy², when present, is a piperazine monosubstituted with a group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In an even further aspect, Cy², when present, is an unsubstituted piperazine.

In various aspects, Cy², when present, is selected from a C6 aryl and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a further aspect, Cy², when present, is selected from a C6 aryl and a C2-C5 heteroaryl, and is substituted with 0, 1, or 2 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a still further aspect, Cy², when present, is selected from a C6 aryl and a C2-C5 heteroaryl, and is substituted with 0 or 1 group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In yet a further aspect, Cy², when present, is selected from a C6 aryl and a C2-C5 heteroaryl, and is monosubstituted with a group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In an even further aspect, Cy², when present, is selected from a C6 aryl and a C2-C5 heteroaryl, and is unsubstituted.

In various aspects, Cy², when present, is a C6 aryl substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a further aspect, Cy², when present, is a C6 aryl substituted with 0, 1, or 2 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a still further aspect, Cy², when present, is a C6 aryl substituted with 0 or 1 group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy. C1-C8 alkoxy, C1-C8 alkylamino, (C1-C8)(C1-C8) dialkylamino, C1-C8 aminoalkyl, —C(O)(C1-C4 alkyl), and Cy³. In yet a further aspect, Cy², when present, is a C6 aryl monosubstituted with a group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In an even further aspect, Cy², when present, is an unsubstituted C6 aryl.

In various aspects, Cy², when present, is a C2-C5 heteroaryl substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. Examples of C2-C5 heteroaryls include, but are not limited to, furan, pyrrole, thiophene, oxazole, isothiazole, pyridine, and triazine. In a further aspect, Cy², when present, is a C2-C5 heteroaryl substituted with 0, 1, or 2 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In a still further aspect, Cy², when present, is a C2-C5 heteroaryl substituted with 0 or 1 group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In yet a further aspect, Cy², when present, is a C2-C5 heteroaryl monosubstituted with a group selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³. In an even further aspect, Cy², when present, is an unsubstituted C₂-C5 heteroaryl.

u. CY³ Groups

In one aspect, Cy³, when present, is a C2-C5 heterocycloalkyl substituted with 0 or 1 group selected from C1-C4 alkyl and —C(O)(C1-C4 alkyl). Examples of C2-C5 heterocycloalkyls include, but are not limited to, thiirane, oxirane, aziridine, thietane, azetidine, oxetane, pyrrolidine, imidazolidine, tetrahydrothiophene, tetrahydrofuran, piperidine, piperazine, thiane, and morpholine. In a further aspect, Cy³, when present, is a C2-C5 heterocycloalkyl monosubstituted with a group selected from C1-C4 alkyl and —C(O)(C1-C4 alkyl). In a still further aspect, Cy³, when present, is an unsubstituted C₂-C5 heterocycloalkyl.

In various aspects, Cy³, when present, is a piperazine substituted with 0 or 1 group selected from C1-C4 alkyl and —C(O)(C1-C4 alkyl). In a further aspect, Cy³, when present, is a piperazine monosubstituted with a group selected from C1-C4 alkyl and —C(O)(C1-C4 alkyl). In a still further aspect, Cy³, when present, is an unsubstituted piperazine.

### 2. Example Compounds

In one aspect, a compound can be present as one or more of the following structures:

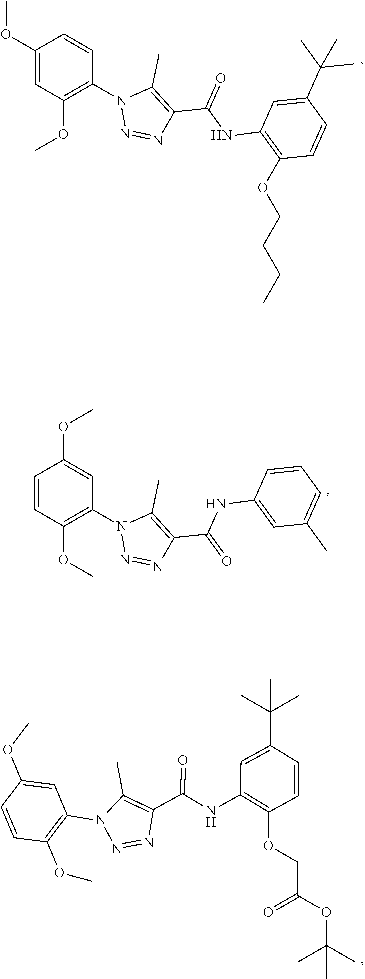 [SMILES: CCCCOc1ccc(C(C)(C)C)cc1NC(=O)c1nnn(-c2ccc(OC)cc2OC)c1C ; COc1ccc(OC)c(-n2nnc(C(=O)Nc3cccc(C)c3)c2C)c1 ; COc1ccc(OC)c(-n2nnc(C(=O)Nc3cc(C(C)(C)C)ccc3OCC(=O)OC(C)(C)C)c2C)c1]

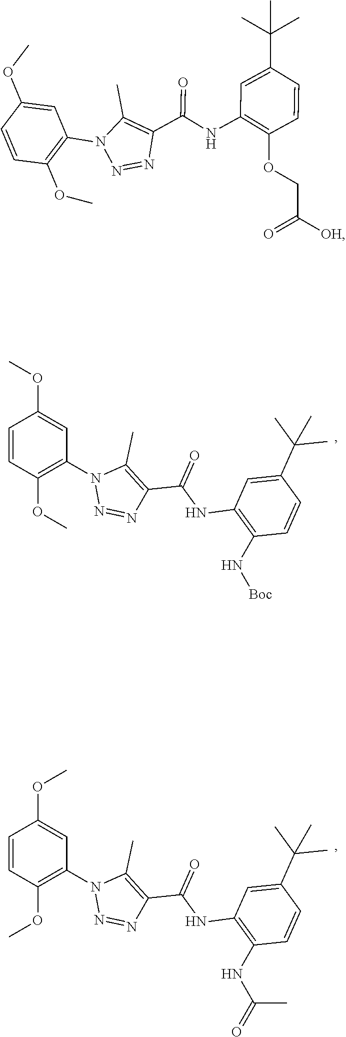 [SMILES: COc1ccc(OC)c(-n2nnc(C(=O)Nc3cc(C(C)(C)C)ccc3OCC(=O)O)c2C)c1 ; COc1ccc(OC)c(-n2nnc(C(=O)Nc3cc(C(C)(C)C)ccc3NC(=O)OC(C)(C)C)c2C)c1 ; COc1ccc(OC)c(-n2nnc(C(=O)Nc3cc(C(C)(C)C)ccc3NC(C)=O)c2C)c1]

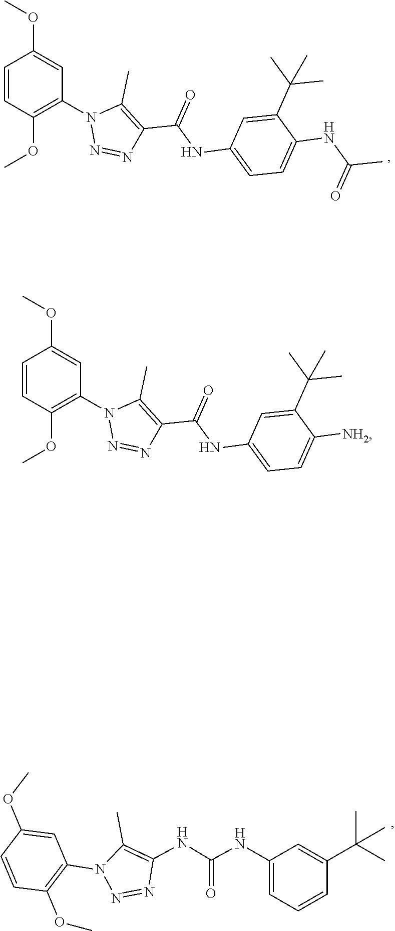 [SMILES: COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1 ; COc1ccc(OC)c(-n2nnc(NC(=O)Nc3cccc(C(C)(C)C)c3)c2C)c1]

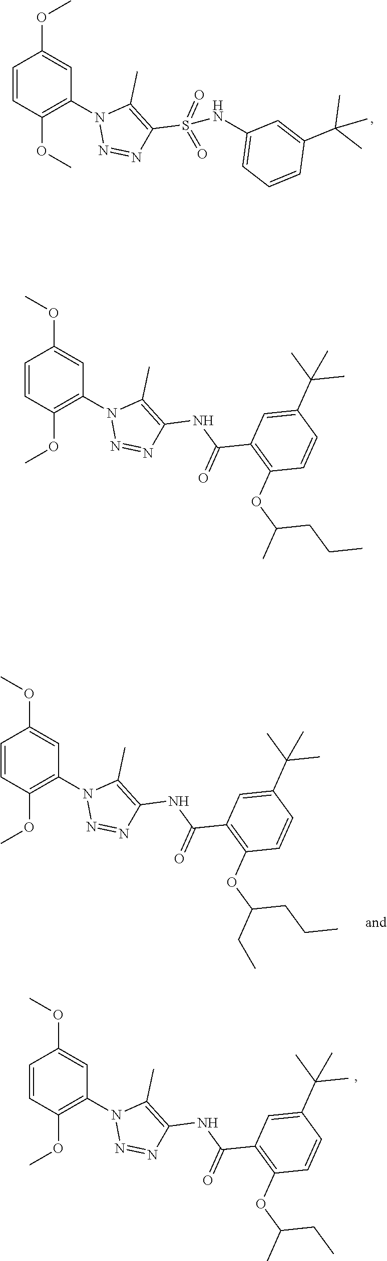 [SMILES: COc1ccc(OC)c(-n2nnc(S(=O)(=O)Nc3cccc(C(C)(C)C)c3)c2C)c1 ; CCCC(C)Oc1ccc(C(C)(C)C)cc1C(=O)Nc1nnn(-c2cc(OC)ccc2OC)c1C ; CCCC(CC)Oc1ccc(C(C)(C)C)cc1C(=O)Nc1nnn(-c2cc(OC)ccc2OC)c1C ; CCC(C)Oc1ccc(C(C)(C)C)cc1C(=O)Nc1nnn(-c2cc(OC)ccc2OC)c1C]

or a pharmaceutically acceptable salt thereof.

In one aspect, a compound can be present as one or more of the following

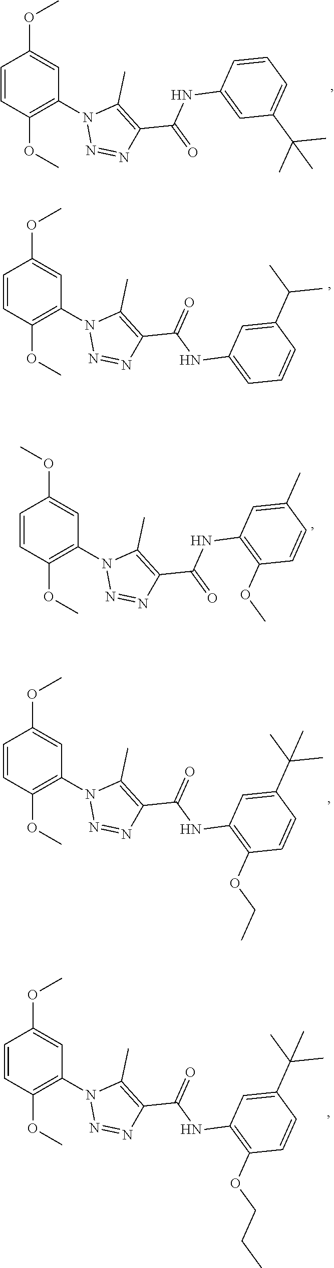

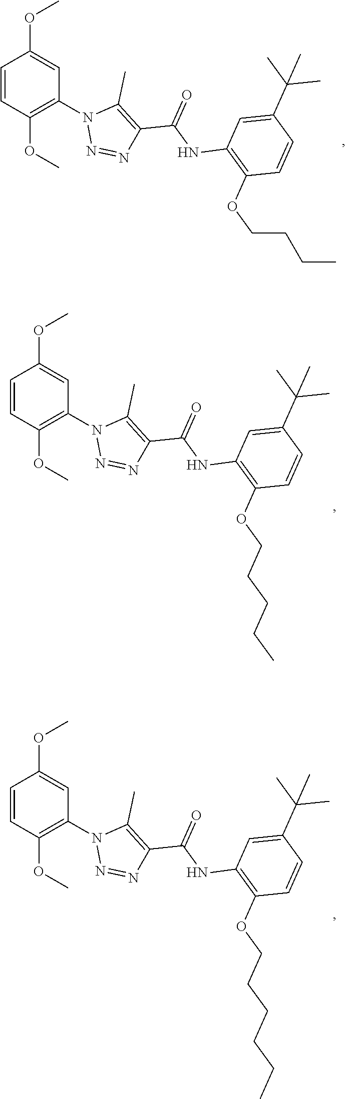 [SMILES: CCCCOc1ccc(C(C)(C)C)cc1NC(=O)c1nnn(-c2cc(OC)ccc2OC)c1C ; CCCCCOc1ccc(C(C)(C)C)cc1NC(=O)c1nnn(-c2cc(OC)ccc2OC)c1C ; CCCCCCOc1ccc(C(C)(C)C)cc1NC(=O)c1nnn(-c2cc(OC)ccc2OC)c1C]

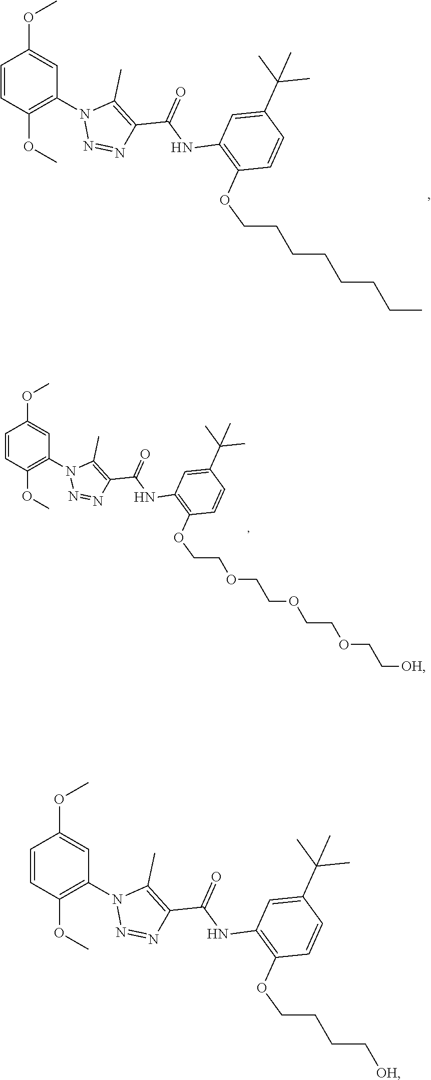 [SMILES: CCCCCCCCOc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OCCOCCOCCOCCO)c2C)c1 ; COc1ccc(OC)c(-n2nnc(C(=O)Nc3cc(C(C)(C)C)ccc3OCCCCO)c2C)c1]

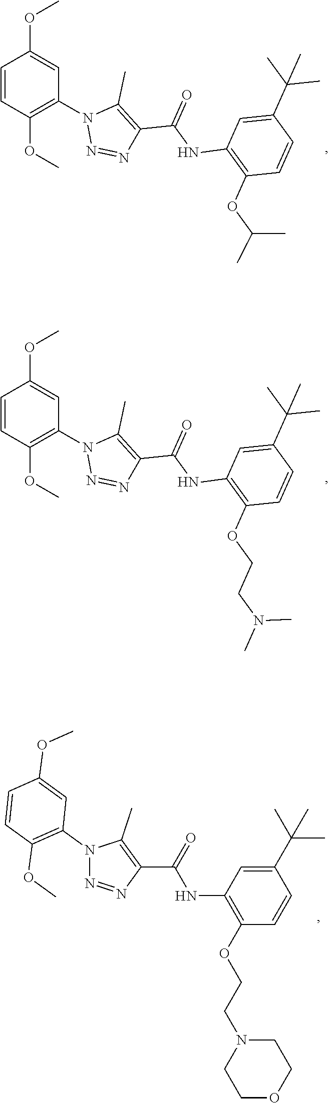 [SMILES: COc1ccc(OC)c(-n2nnc(C(=O)Nc3cc(C(C)(C)C)ccc3OC(C)C)c2C)c1 ; COc1ccc(OC)c(-n2nnc(C(=O)Nc3cc(C(C)(C)C)ccc3OCCN(C)C)c2C)c1 ; COc1ccc(OC)c(-n2nnc(C(=O)Nc3cc(C(C)(C)C)ccc3OCCN3CCOCC3)c2C)c1]

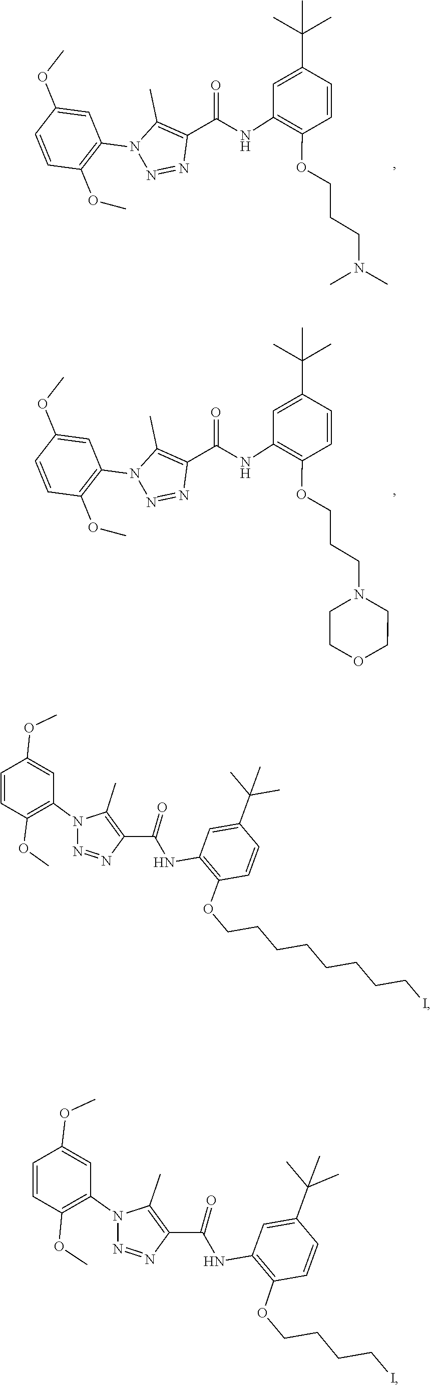 [SMILES: COc1ccc(OC)c(-n2nnc(C(=O)Nc3cc(C(C)(C)C)ccc3OCCCN(C)C)c2C)c1 ; COc1ccc(OC)c(-n2nnc(C(=O)Nc3cc(C(C)(C)C)ccc3OCCCN3CCOCC3)c2C)c1 ; COc1ccc(OC)c(-n2nnc(C(=O)Nc3cc(C(C)(C)C)ccc3OCCCCCCCCI)c2C)c1 ; COc1ccc(OC)c(-n2nnc(C(=O)Nc3cc(C(C)(C)C)ccc3OCCCCI)c2C)c1]

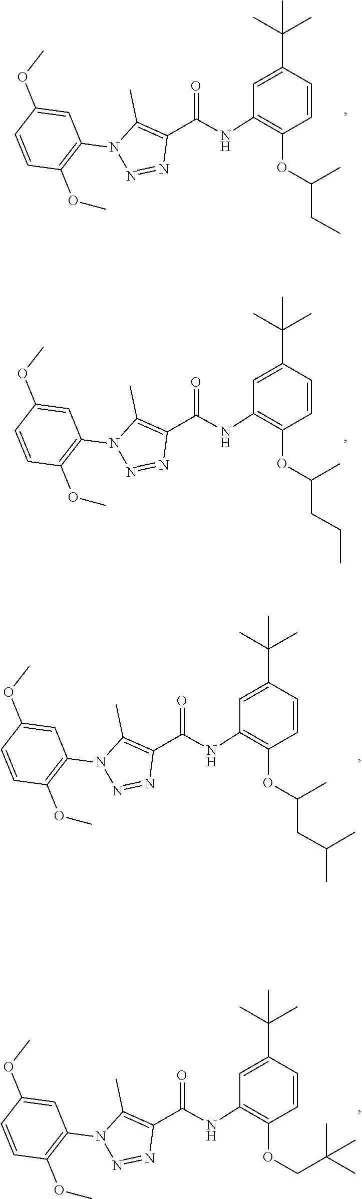 [SMILES: CCC(C)Oc1ccc(C(C)(C)C)cc1NC(=O)c1nnn(-c2cc(OC)ccc2OC)c1C ; CCCC(C)Oc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OC(C)CC(C)C)c2C)c1 ; COc1ccc(OC)c(-n2nnc(C(=O)Nc3cc(C(C)(C)C)ccc3OCC(C)(C)C)c2C)c1]

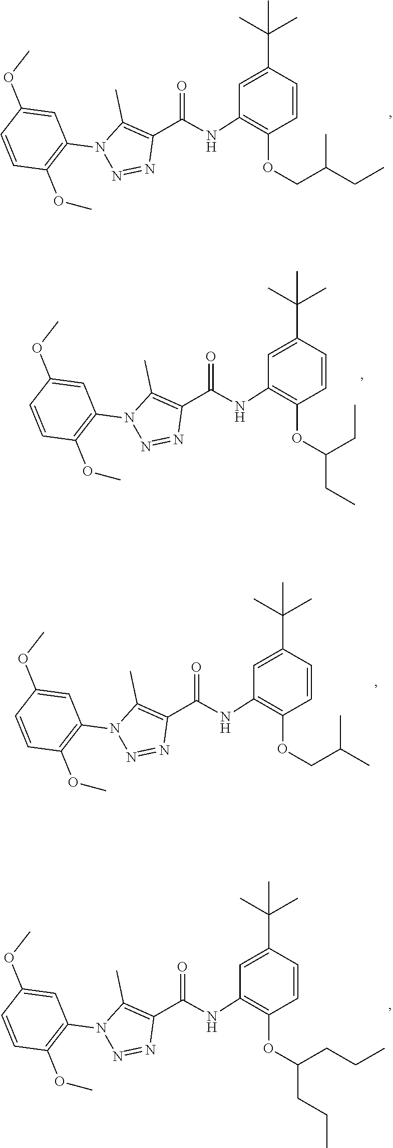 [SMILES: CCC(C)COc1ccc(C(C)(C)C)cc1NC(=O)c1nnn(-c2cc(OC)ccc2OC)c1C ; CCC(CC)Oc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OCC(C)C)c2C)c1 ; CCCC(CCC)Oc1ccc(C(C)(C)C)cc1NC(=O)c1nnn(-c2cc(OC)ccc2OC)c1C]

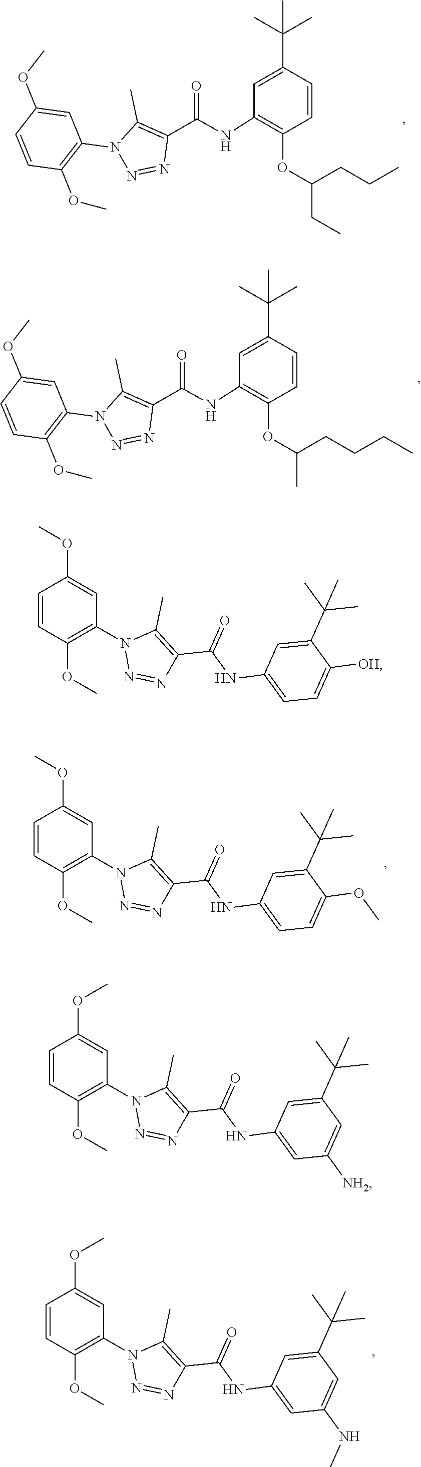

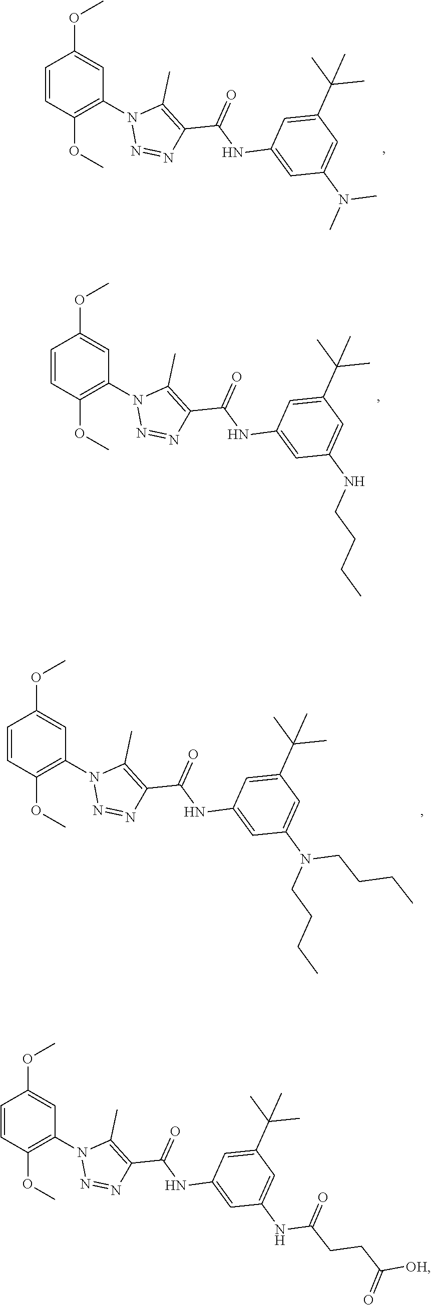 [SMILES: COc1ccc(OC)c(-n2nnc(C(=O)Nc3cc(N(C)C)cc(C(C)(C)C)c3)c2C)c1 ; CCCCNc1cc(NC(=O)c2nnn(-c3cc(OC)ccc3OC)c2C)cc(C(C)(C)C)c1 ; CCCCN(CCCC)c1cc(NC(=O)c2nnn(-c3cc(OC)ccc3OC)c2C)cc(C(C)(C)C)c1 ; COc1ccc(OC)c(-n2nnc(C(=O)Nc3cc(NC(=O)CCC(=O)O)cc(C(C)(C)C)c3)c2C)c1]

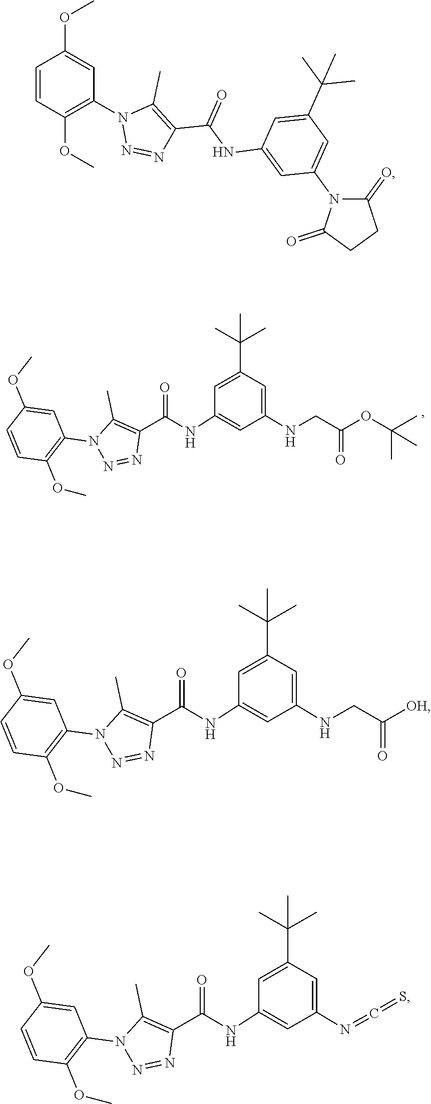 [SMILES: COc1ccc(OC)c(-n2nnc(C(=O)Nc3cc(N4C(=O)CCC4=O)cc(C(C)(C)C)c3)c2C)c1 ; COc1ccc(OC)c(-n2nnc(C(=O)Nc3cc(NCC(=O)OC(C)(C)C)cc(C(C)(C)C)c3)c2C)c1 ; COc1ccc(OC)c(-n2nnc(C(=O)Nc3cc(NCC(=O)O)cc(C(C)(C)C)c3)c2C)c1 ; COc1ccc(OC)c(-n2nnc(C(=O)Nc3cc(N=C=S)cc(C(C)(C)C)c3)c2C)c1]

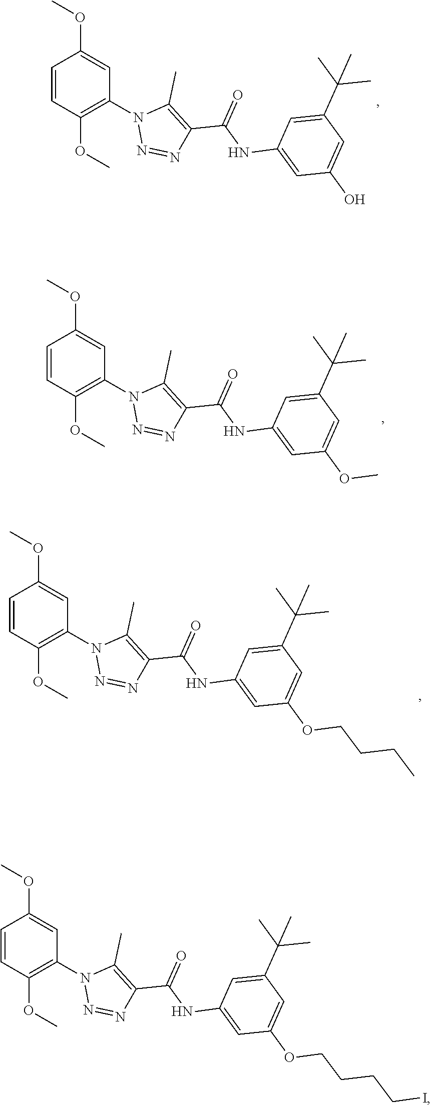

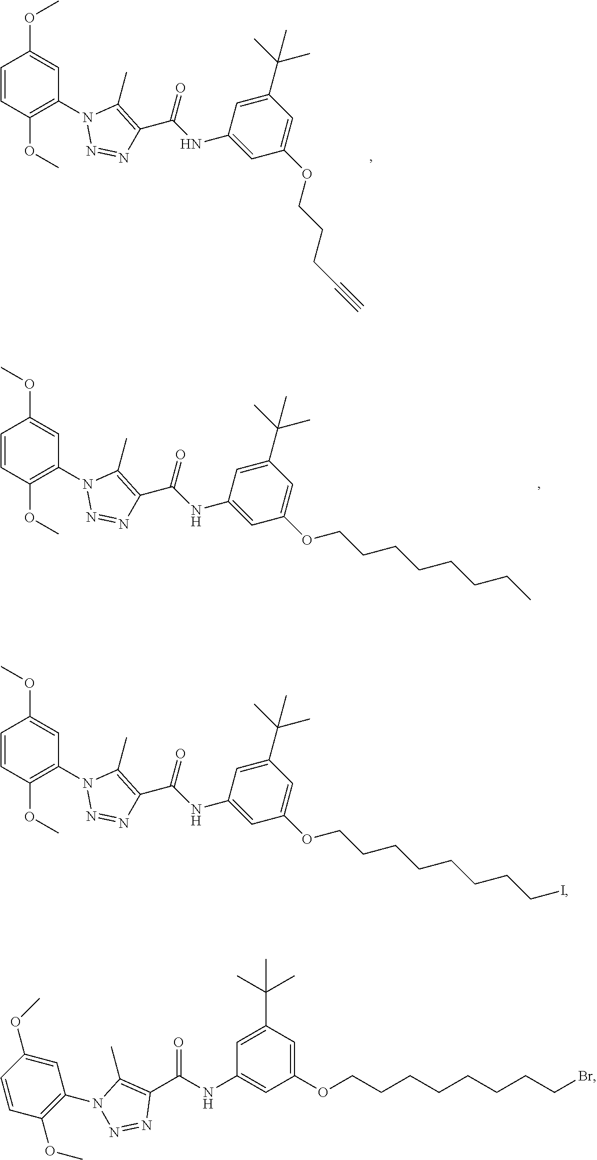 [SMILES: C#CCCCOc1cc(NC(=O)c2nnn(-c3cc(OC)ccc3OC)c2C)cc(C(C)(C)C)c1 ; CCCCCCCCOc1cc(NC(=O)c2nnn(-c3cc(OC)ccc3OC)c2C)cc(C(C)(C)C)c1 ; COc1ccc(OC)c(-n2nnc(C(=O)Nc3cc(OCCCCCCCCI)cc(C(C)(C)C)c3)c2C)c1 ; COc1ccc(OC)c(-n2nnc(C(=O)Nc3cc(OCCCCCCCCBr)cc(C(C)(C)C)c3)c2C)c1]

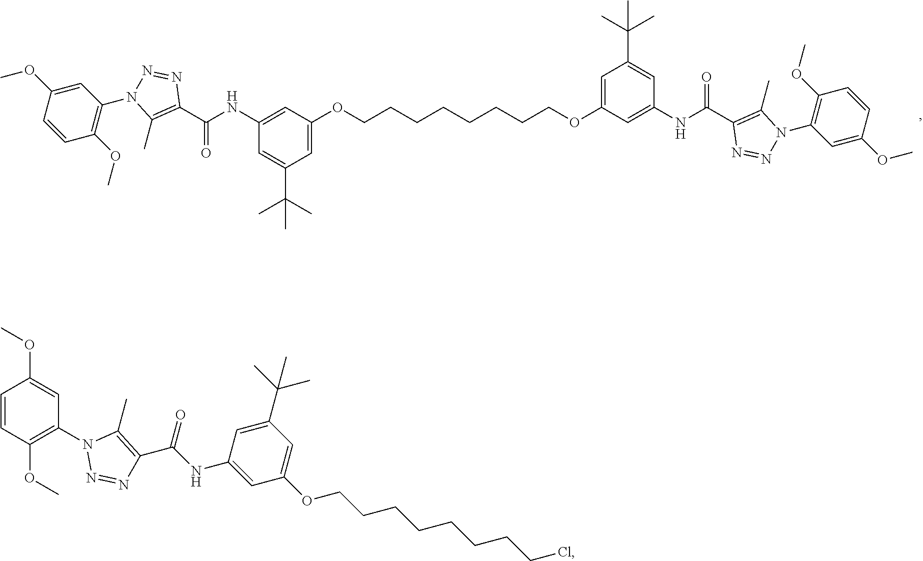

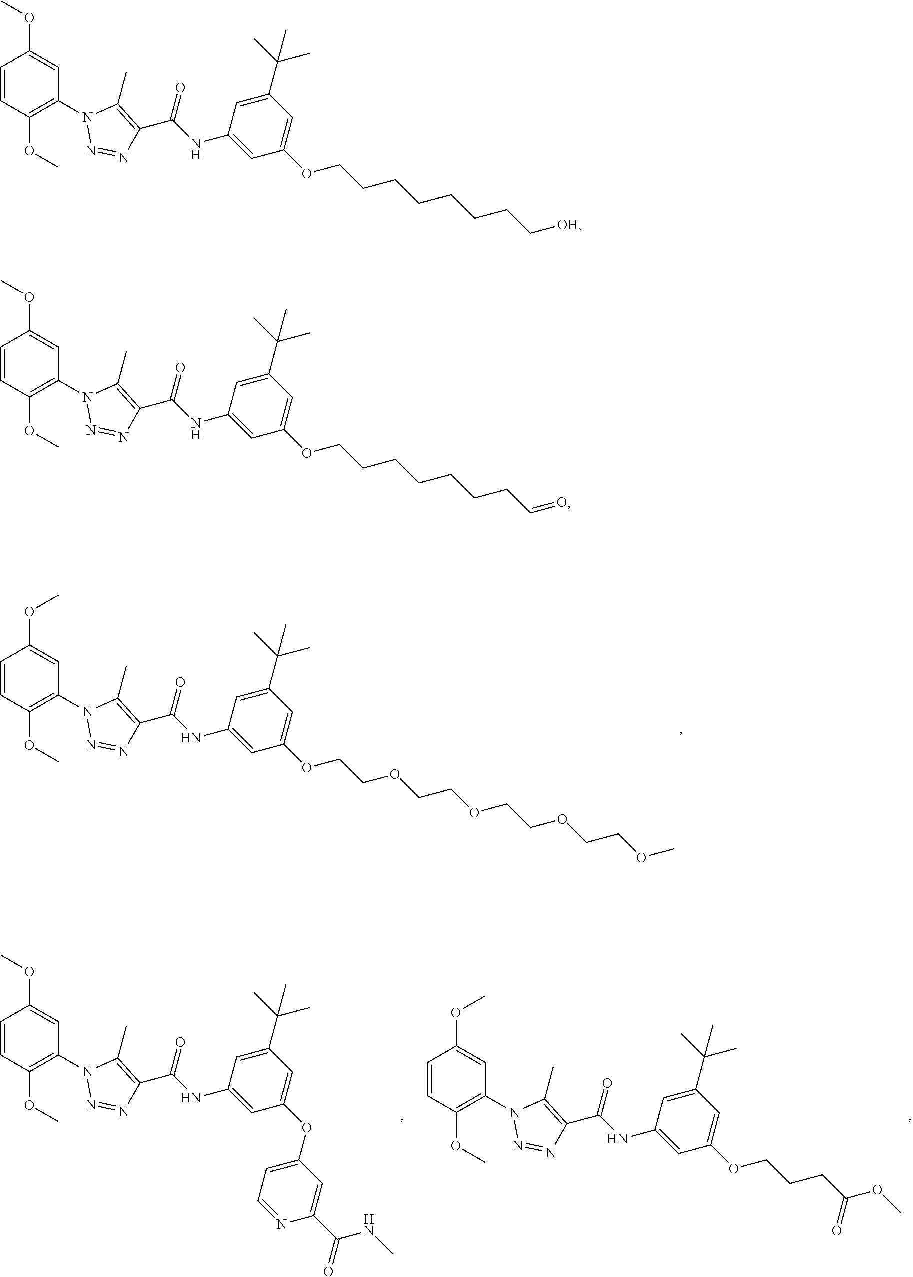 [SMILES: COc1ccc(OC)c(-n2nnc(C(=O)Nc3cc(OCCCCCCCCO)cc(C(C)(C)C)c3)c2C)c1 ; COc1ccc(OC)c(-n2nnc(C(=O)Nc3cc(OCCCCCCCC=O)cc(C(C)(C)C)c3)c2C)c1 ; COCCOCCOCCOCCOc1cc(NC(=O)c2nnn(-c3cc(OC)ccc3OC)c2C)cc(C(C)(C)C)c1 ; CNC(=O)c1cc(Oc2cc(NC(=O)c3nnn(-c4cc(OC)ccc4OC)c3C)cc(C(C)(C)C)c2)ccn1 ; COC(=O)CCCOc1cc(NC(=O)c2nnn(-c3cc(OC)ccc3OC)c2C)cc(C(C)(C)C)c1]

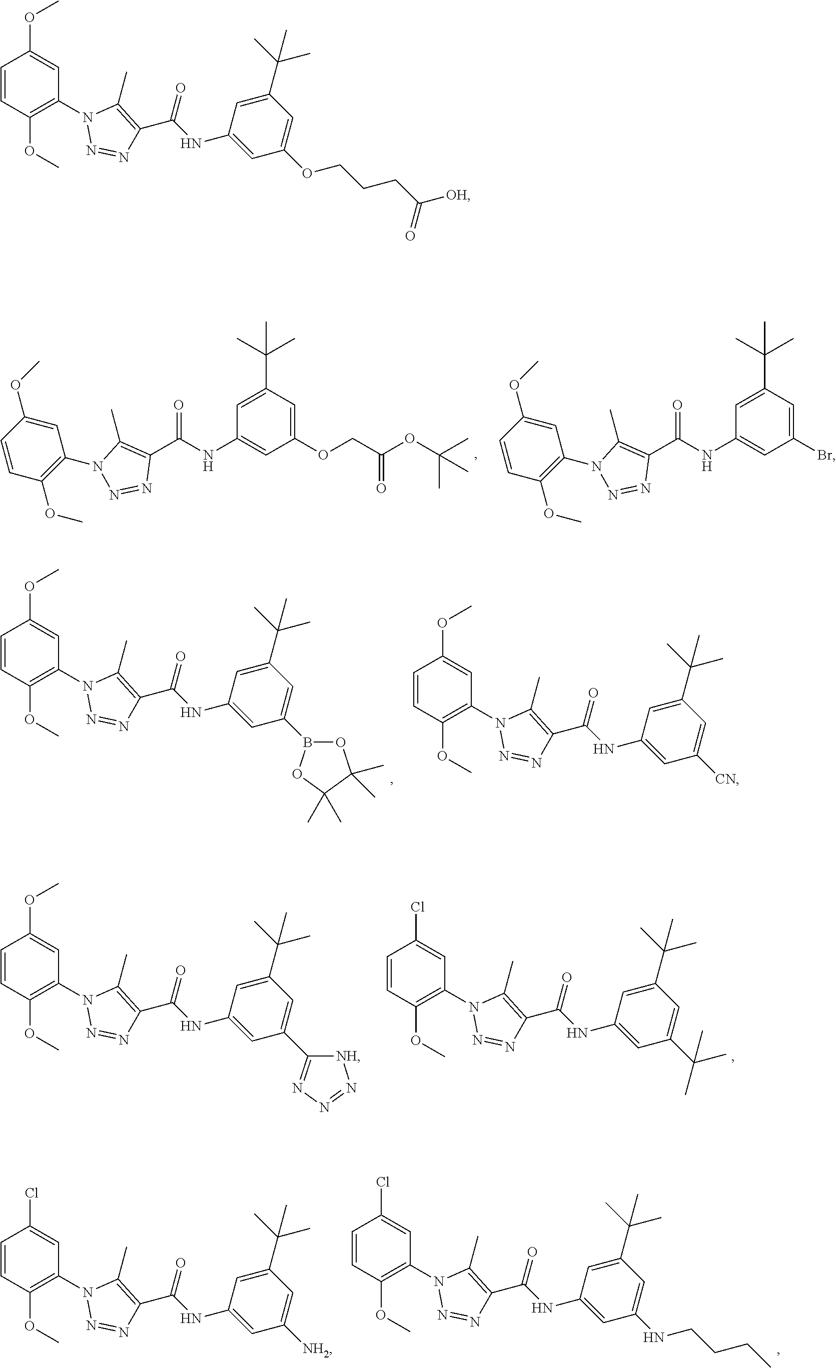

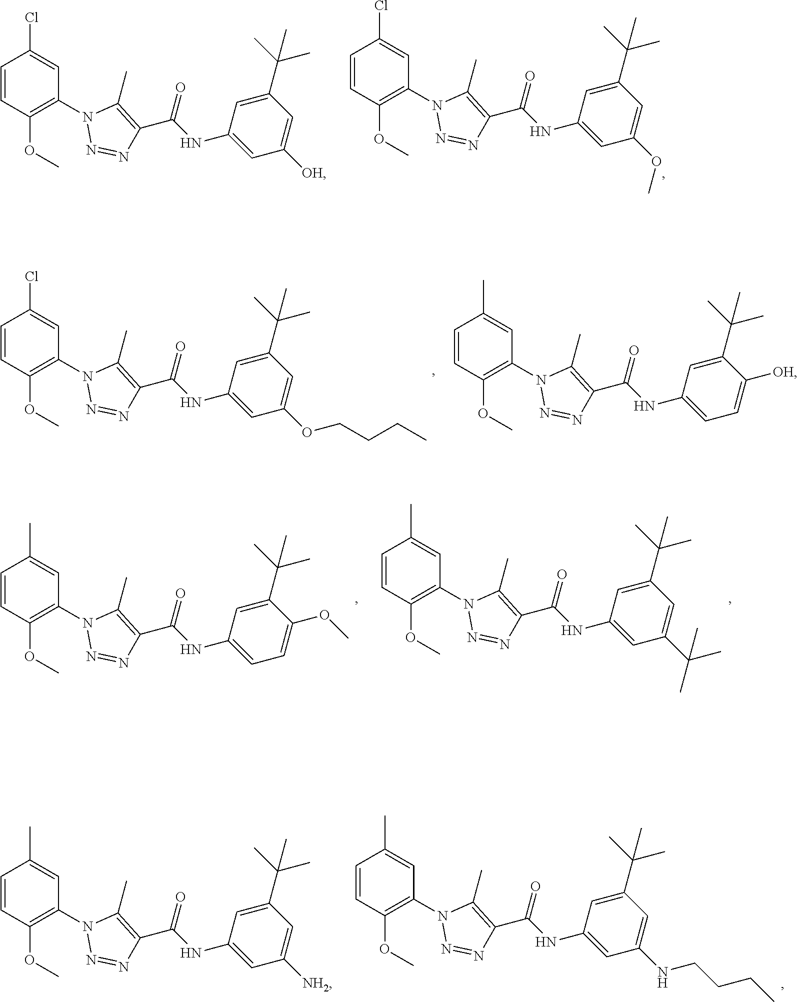

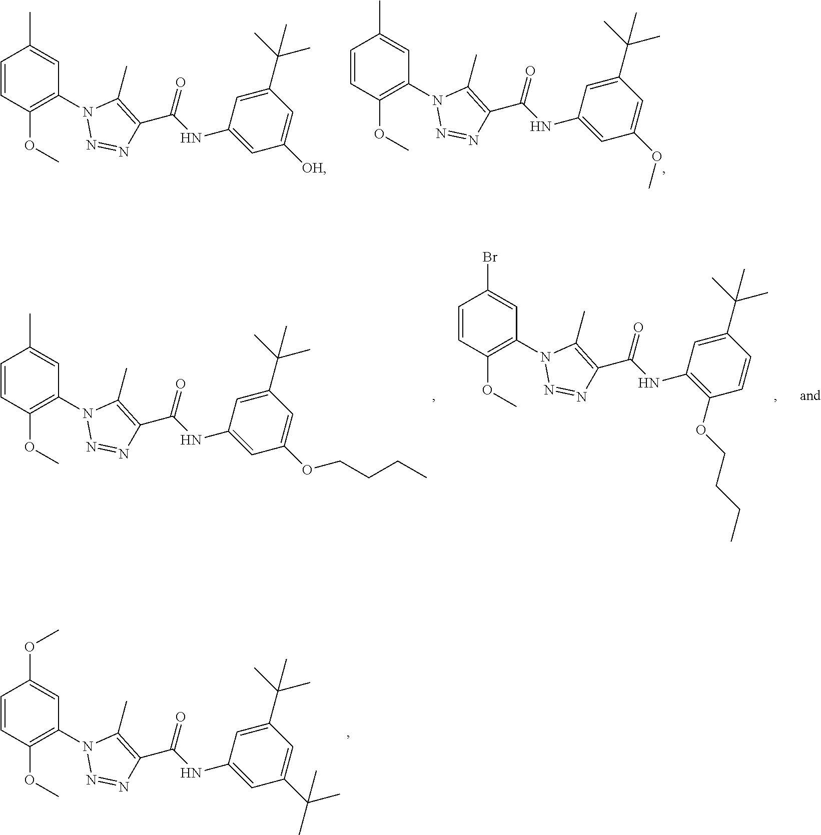

or a pharmaceutically acceptable salt thereof.

In one aspect, a compound can be present as one or more of the following structures:

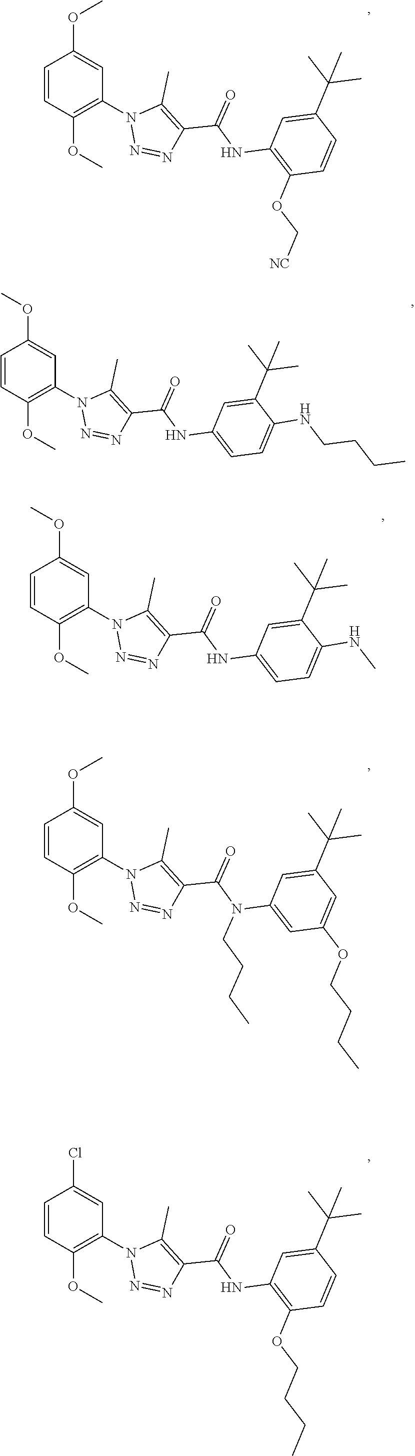 [SMILES: COc1ccc(OC)c(-n2nnc(C(=O)Nc3cc(C(C)(C)C)ccc3OCC#N)c2C)c1 ; CCCCNc1ccc(NC(=O)c2nnn(-c3cc(OC)ccc3OC)c2C)cc1C(C)(C)C ; CNc1ccc(NC(=O)c2nnn(-c3cc(OC)ccc3OC)c2C)cc1C(C)(C)C ; CCCCOc1cc(N(CCCC)C(=O)c2nnn(-c3cc(OC)ccc3OC)c2C)cc(C(C)(C)C)c1 ; CCCCOc1ccc(C(C)(C)C)cc1NC(=O)c1nnn(-c2cc(Cl)ccc2OC)c1C]

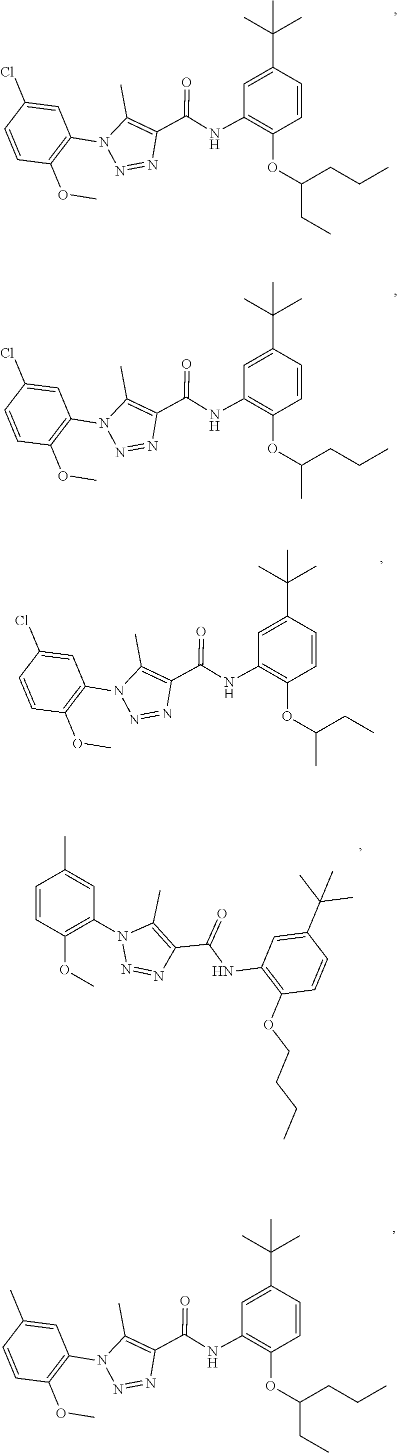 [SMILES: CCCC(CC)Oc1ccc(C(C)(C)C)cc1NC(=O)c1nnn(-c2cc(Cl)ccc2OC)c1C ; CCCC(C)Oc1ccc(C(C)(C)C)cc1NC(=O)c1nnn(-c2cc(Cl)ccc2OC)c1C ; CCC(C)Oc1ccc(C(C)(C)C)cc1NC(=O)c1nnn(-c2cc(Cl)ccc2OC)c1C ; CCCCOc1ccc(C(C)(C)C)cc1NC(=O)c1nnn(-c2cc(C)ccc2OC)c1C ; CCCC(CC)Oc1ccc(C(C)(C)C)cc1NC(=O)c1nnn(-c2cc(C)ccc2OC)c1C]

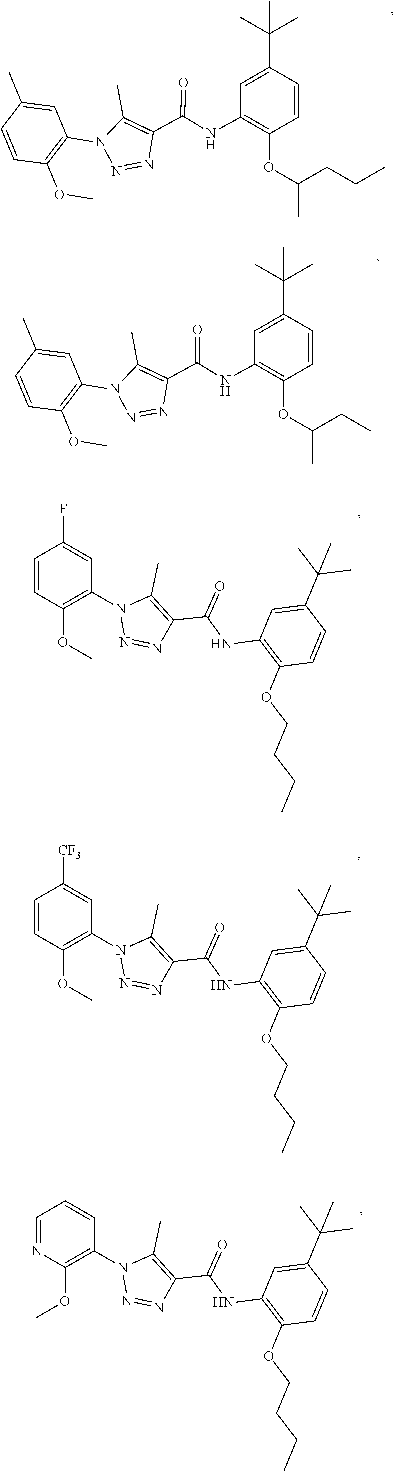

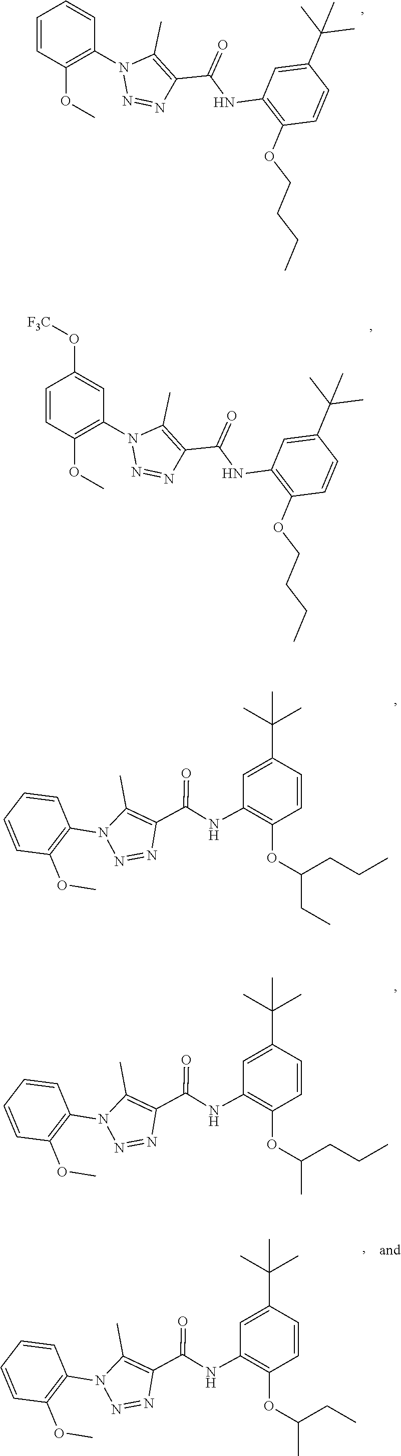 [SMILES: CCCCOc1ccc(C(C)(C)C)cc1NC(=O)c1nnn(-c2ccccc2OC)c1C ; CCCCOc1ccc(C(C)(C)C)cc1NC(=O)c1nnn(-c2cc(OC(F)(F)F)ccc2OC)c1C ; CCCC(CC)Oc1ccc(C(C)(C)C)cc1NC(=O)c1nnn(-c2ccccc2OC)c1C ; CCCC(C)Oc1ccc(C(C)(C)C)cc1NC(=O)c1nnn(-c2ccccc2OC)c1C ; CCC(C)Oc1ccc(C(C)(C)C)cc1NC(=O)c1nnn(-c2ccccc2OC)c1C]

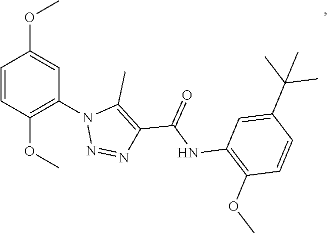 [SMILES: COc1ccc(OC)c(-n2nnc(C(=O)Nc3cc(C(C)(C)C)ccc3OC)c2C)c1]

or a pharmaceutically acceptable salt thereof.

In one aspect, a compound can be present as one or more of the following

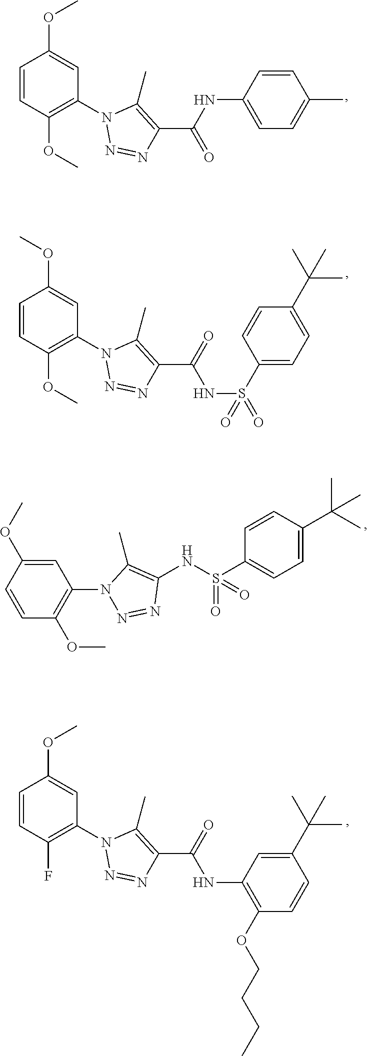 [SMILES: COc1ccc(OC)c(-n2nnc(C(=O)Nc3ccc(C)cc3)c2C)c1 ; COc1ccc(OC)c(-n2nnc(C(=O)NS(=O)(=O)c3ccc(C(C)(C)C)cc3)c2C)c1 ; COc1ccc(OC)c(-n2nnc(NS(=O)(=O)c3ccc(C(C)(C)C)cc3)c2C)c1 ; CCCCOc1ccc(C(C)(C)C)cc1NC(=O)c1nnn(-c2cc(OC)ccc2F)c1C]

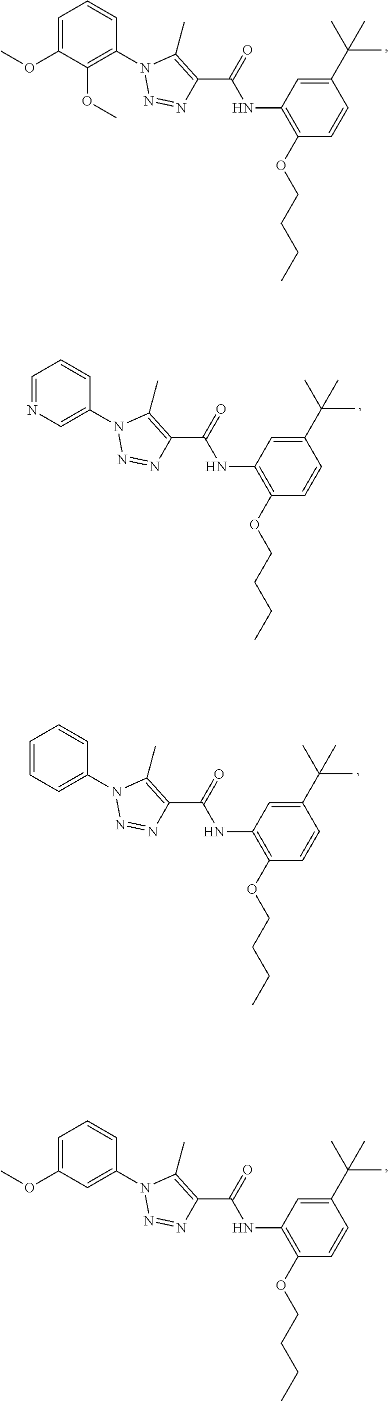

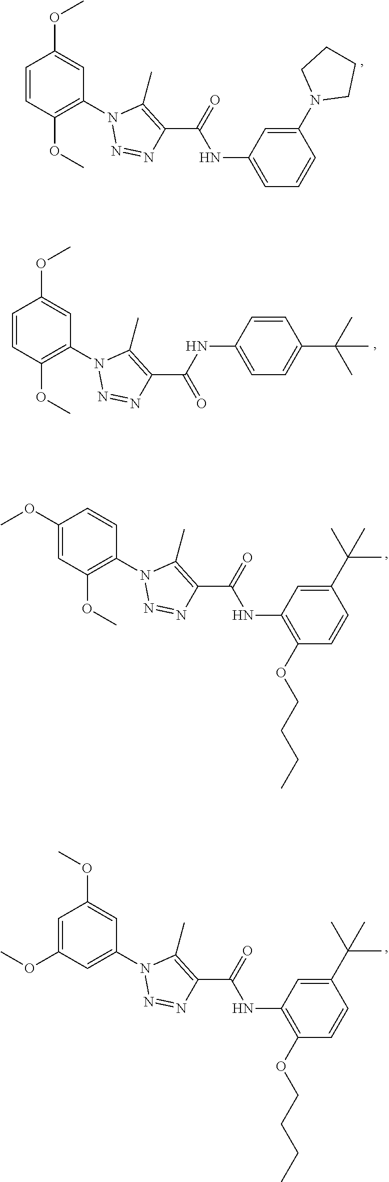

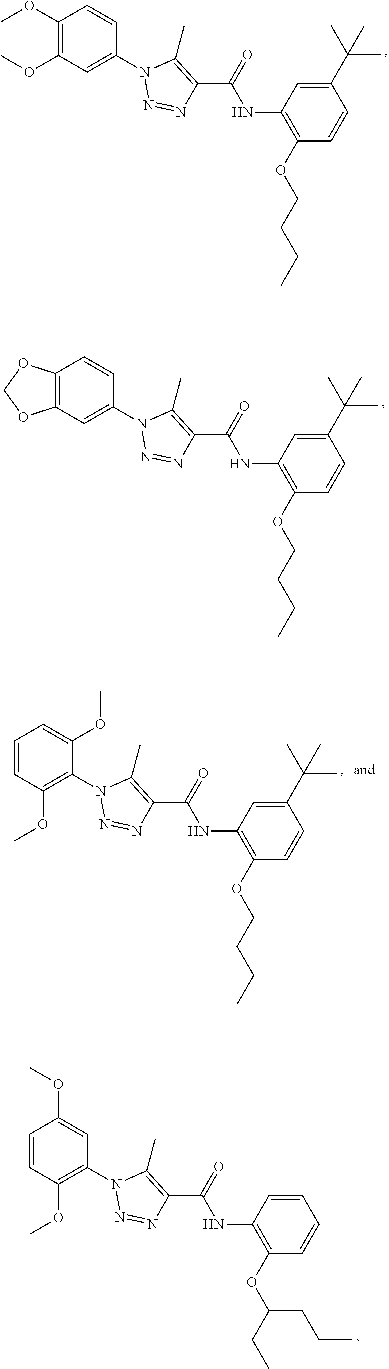 [SMILES: CCCCOc1ccc(C(C)(C)C)cc1NC(=O)c1nnn(-c2ccc(OC)c(OC)c2)c1C ; CCCCOc1ccc(C(C)(C)C)cc1NC(=O)c1nnn(-c2ccc3c(c2)OCO3)c1C ; CCCCOc1ccc(C(C)(C)C)cc1NC(=O)c1nnn(-c2c(OC)cccc2OC)c1C ; CCCC(CC)Oc1ccccc1NC(=O)c1nnn(-c2cc(OC)ccc2OC)c1C]

or a pharmaceutically acceptable salt thereof.

In one aspect, a compound can be present as one or more of the following structures:

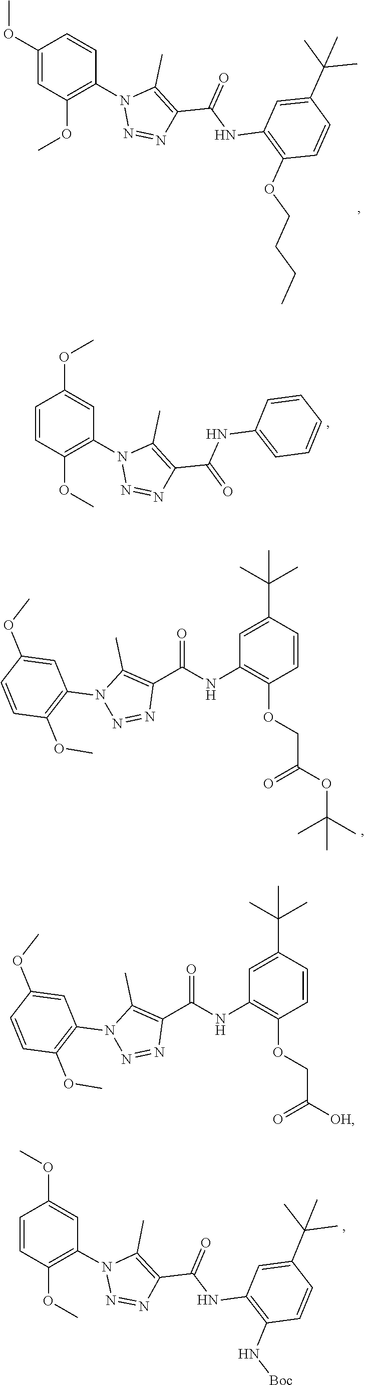 [SMILES: CCCCOc1ccc(C(C)(C)C)cc1NC(=O)c1nnn(-c2ccc(OC)cc2OC)c1C ; COc1ccc(OC)c(-n2nnc(C(=O)Nc3ccccc3)c2C)c1 ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1 ; COc1ccc(OC)c(-n2nnc(C(=O)Nc3cc(C(C)(C)C)ccc3NC(=O)OC(C)(C)C)c2C)c1]

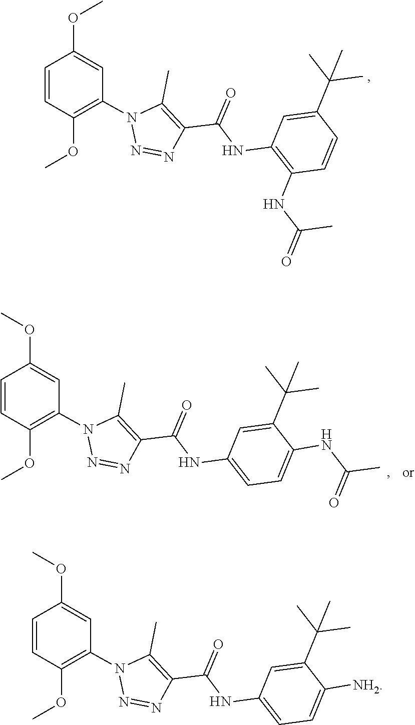 [SMILES: COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1]

or a pharmaceutically acceptable salt thereof.

In one aspect, a compound can be present as one or more of the following structures:

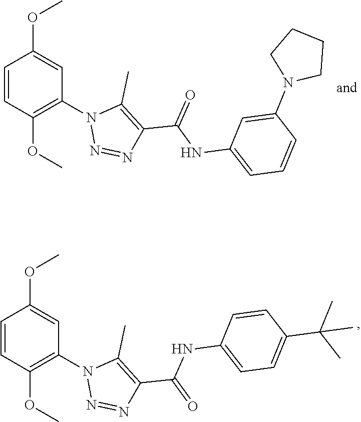 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

In one aspect, a compound can be present as one or more of the following

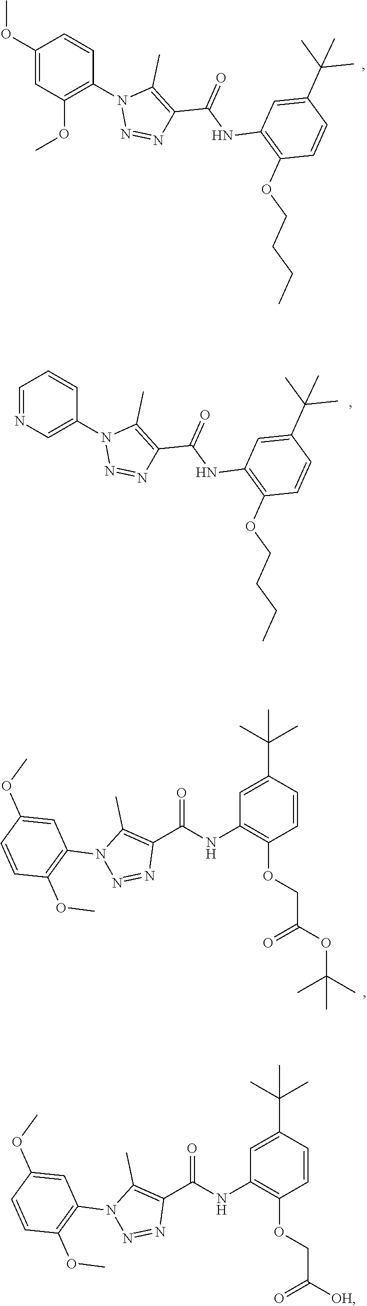 [SMILES: CCCCOc1ccc(C(C)(C)C)cc1NC(=O)c1nnn(-c2ccc(OC)cc2OC)c1C ; CCCCOc1ccc(C(C)(C)C)cc1NC(=O)c1nnn(-c2cccnc2)c1C ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1]

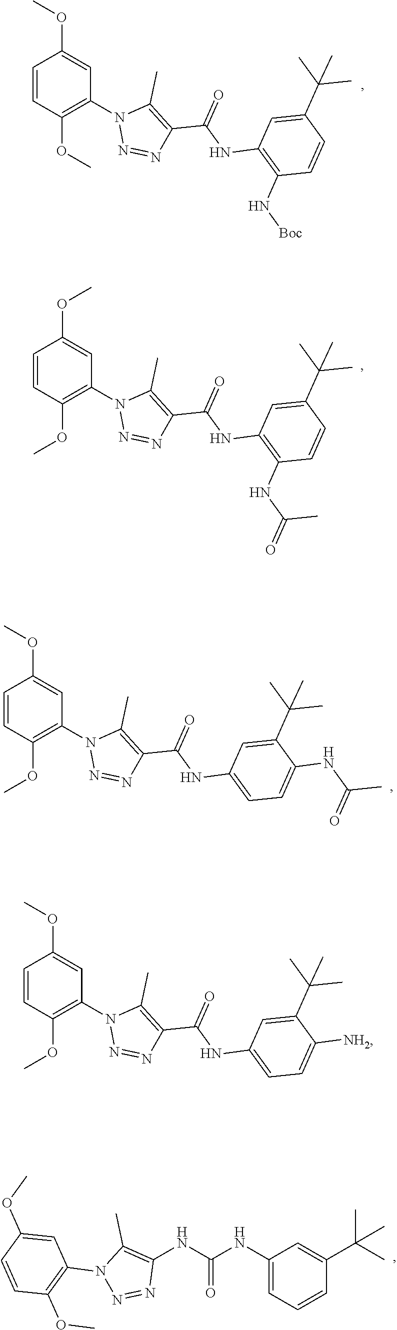 [SMILES: COc1ccc(OC)c(-n2nnc(C(=O)Nc3cc(C(C)(C)C)ccc3NC(=O)OC(C)(C)C)c2C)c1 ; COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1 ; COc1ccc(OC)c(-n2nnc(NC(=O)Nc3cccc(C(C)(C)C)c3)c2C)c1]

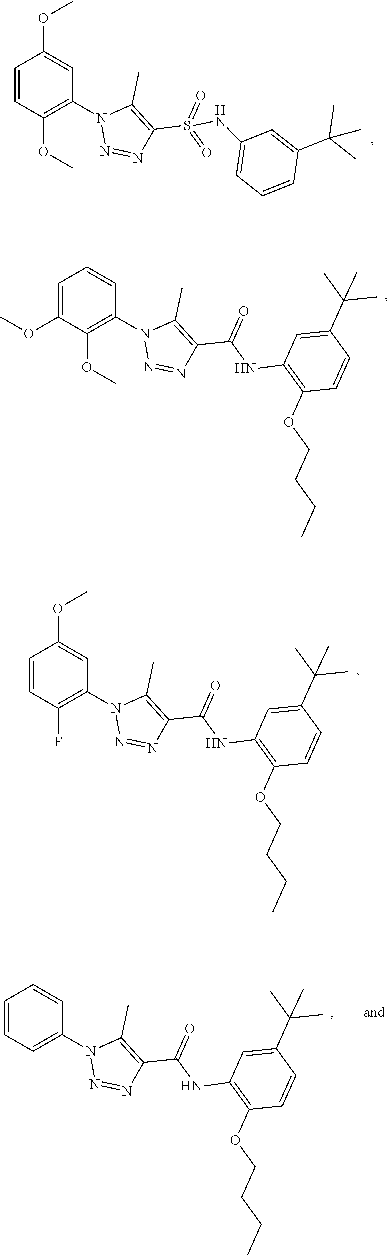

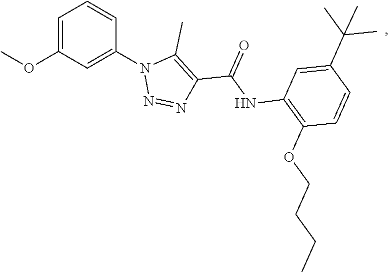 [SMILES: CCCCOc1ccc(C(C)(C)C)cc1NC(=O)c1nnn(-c2cccc(OC)c2)c1C]

or a pharmaceutically acceptable salt thereof.

In one aspect, a compound can be present as one or more of the following structures:

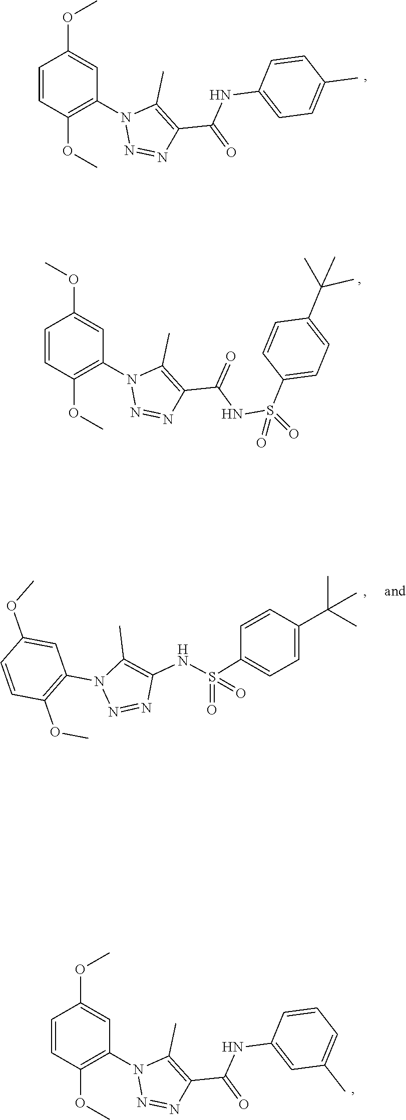 [SMILES: COc1ccc(OC)c(-n2nnc(C(=O)Nc3ccc(C)cc3)c2C)c1 ; COc1ccc(OC)c(-n2nnc(C(=O)NS(=O)(=O)c3ccc(C(C)(C)C)cc3)c2C)c1 ; COc1ccc(OC)c(-n2nnc(NS(=O)(=O)c3ccc(C(C)(C)C)cc3)c2C)c1 ; COc1ccc(OC)c(-n2nnc(C(=O)Nc3cccc(C)c3)c2C)c1]

or a pharmaceutically acceptable salt thereof.

In one aspect, a compound can be present as one or more of the following

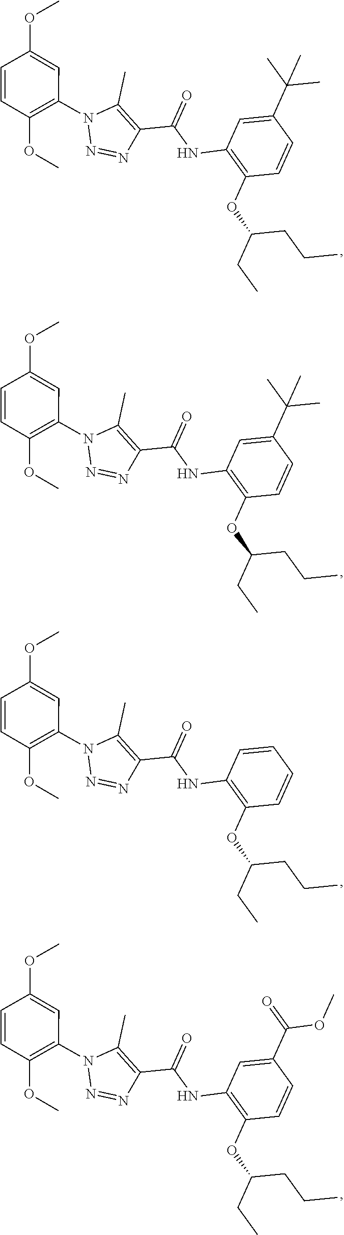 [SMILES: CCC[C@H](CC)Oc1ccc(C(C)(C)C)cc1NC(=O)c1nnn(-c2cc(OC)ccc2OC)c1C ; CCC[C@@H](CC)Oc1ccc(C(C)(C)C)cc1NC(=O)c1nnn(-c2cc(OC)ccc2OC)c1C ; CCC[C@H](CC)Oc1ccccc1NC(=O)c1nnn(-c2cc(OC)ccc2OC)c1C ; CCC[C@H](CC)Oc1ccc(C(=O)OC)cc1NC(=O)c1nnn(-c2cc(OC)ccc2OC)c1C]

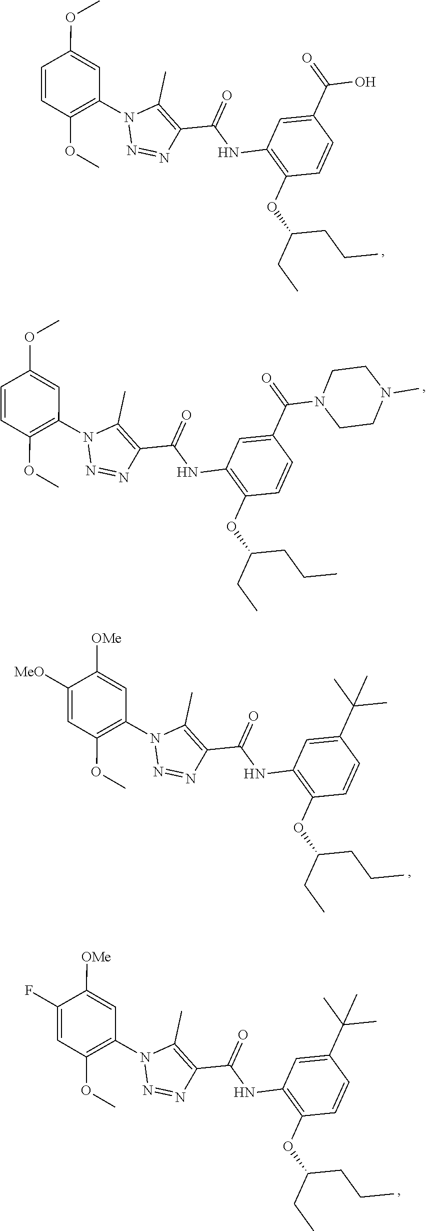 [SMILES: CCC[C@H](CC)Oc1ccc(C(=O)O)cc1NC(=O)c1nnn(-c2cc(OC)ccc2OC)c1C ; CCC[C@H](CC)Oc1ccc(C(=O)N2CCN(C)CC2)cc1NC(=O)c1nnn(-c2cc(OC)ccc2OC)c1C ; CCC[C@H](CC)Oc1ccc(C(C)(C)C)cc1NC(=O)c1nnn(-c2cc(OC)c(OC)cc2OC)c1C ; CCC[C@H](CC)Oc1ccc(C(C)(C)C)cc1NC(=O)c1nnn(-c2cc(OC)c(F)cc2OC)c1C]

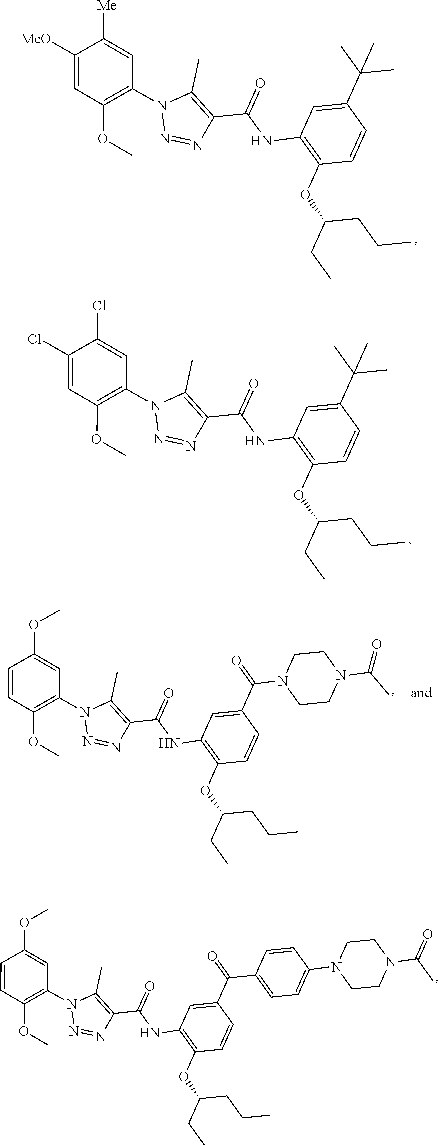 [SMILES: CCC[C@H](CC)Oc1ccc(C(C)(C)C)cc1NC(=O)c1nnn(-c2cc(C)c(OC)cc2OC)c1C ; CCC[C@H](CC)Oc1ccc(C(C)(C)C)cc1NC(=O)c1nnn(-c2cc(Cl)c(Cl)cc2OC)c1C ; CCC[C@H](CC)Oc1ccc(C(=O)N2CCN(C(C)=O)CC2)cc1NC(=O)c1nnn(-c2cc(OC)ccc2OC)c1C ; CCC[C@H](CC)Oc1ccc(C(=O)c2ccc(N3CCN(C(C)=O)CC3)cc2)cc1NC(=O)c1nnn(-c2cc(OC)ccc2OC)c1C]

or a pharmaceutically acceptable salt thereof.

### C. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-VIII, as described and exemplified below. The following examples are provided so that the invention might be more fully understood, are illustrative only, and should not be construed as limiting.

### 1. Route I

In one aspect, 1,4,5-substituted 1,2,3-triazole compounds can be prepared as shown below.

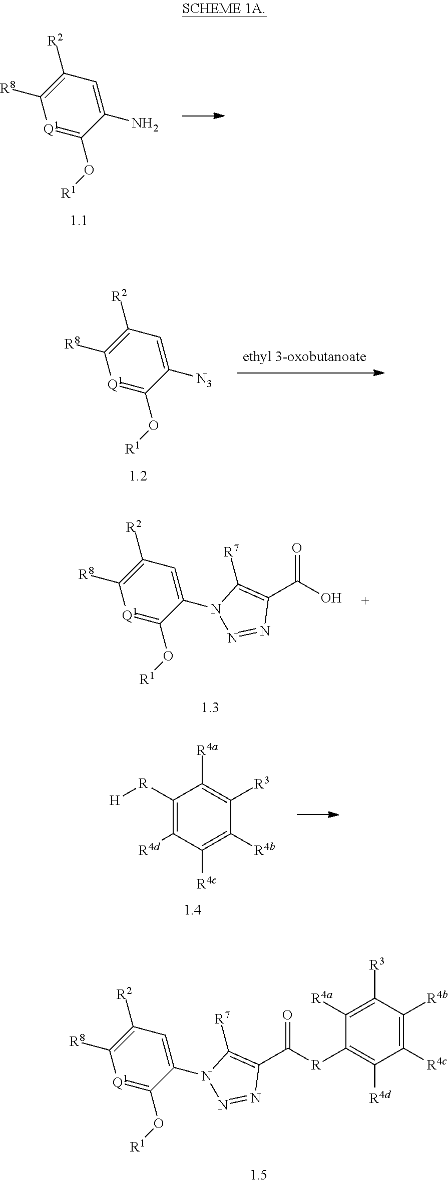

Compounds are represented in generic form, wherein R is —NR¹⁰— or —S—, and with substituents as noted in compound descriptions elsewhere herein. As would be understood by one of ordinary skill in the art, a similar reaction could also be performed in which the —CO₂H group is replaced with a —SO₂H and the —RH group is replaced by a —N(R¹⁰)H, thereby generated a —SO₂N(R¹⁰)— linker moiety. A more specific example is set forth below.

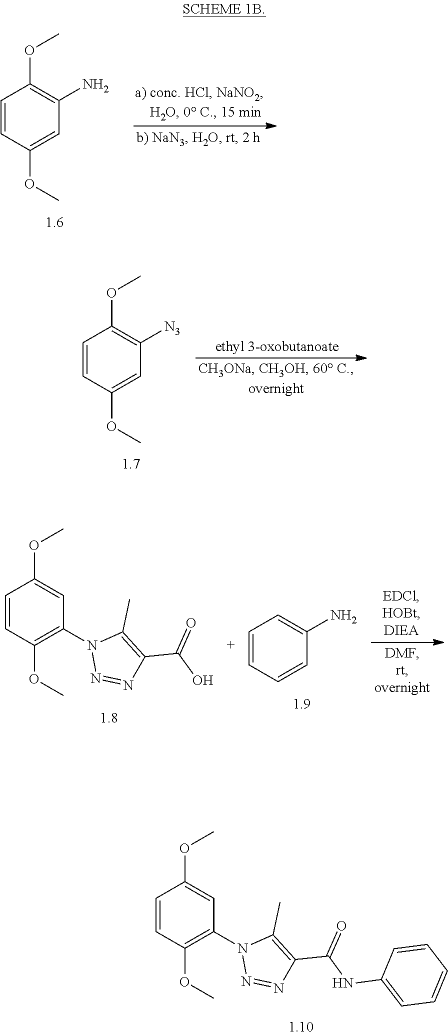 [SMILES: COc1ccc(OC)c(N)c1 ; COc1ccc(OC)c(N=[N+]=[N-])c1 ; COc1ccc(OC)c(-n2nnc(C(=O)O)c2C)c1 ; Nc1ccccc1 ; COc1ccc(OC)c(-n2nnc(C(=O)Nc3ccccc3)c2C)c1]

In one aspect, compounds of type 1.10, and similar compounds, can be prepared according to reaction Scheme 1B above. Thus, compounds of type 1.2 can be prepared by oxidation of an appropriate aniline, e.g., 1.1 as shown above. Appropriate anilines are commercially available or prepared by methods known to one skilled in the art. The oxidation is carried out in the presence of an appropriate acid, e.g., concentrated hydrochloric acid, and an appropriate oxidizing agent, e.g., sodium nitrite, followed by addition of an appropriate azide, e.g., sodium azide. Compounds of type 1.3 can be prepared by cyclization of an appropriate azide, e.g., 1.2 as shown above, with ethyl 3-oxobutanoate. The cyclization is carried out in the presence of an appropriate base, e.g., sodium methoxide, in an appropriate solvent, e.g., methanol, at an appropriate temperature, e.g., 60° C. Compounds of type 1.5 can be prepared by a coupling reaction between an appropriate triazole, e.g., 1.3 as shown above, and an appropriate aniline, e.g., 1.4 as shown above. Appropriate anilines are commercially available or prepared by methods known to one skilled in the art. The coupling reaction is carried out in the presence of an appropriate coupling agent, e.g., 1-ethyl-3-(3-dimethylaminopropyl)carbodiimide (EDCI), an appropriate activating agent, e.g., hydroxybenzotriazole (HOBt), and an appropriate base, e.g., N, N-diisopropylethylamine (DIEA), in an appropriate solvent, e.g., dimethylformamide (DMF). As can be appreciated by one skilled in the art, the above reaction provides an example of a generalized approach wherein compounds similar in structure to the specific reactants above (compounds similar to compounds of type 1.1, 1.2, 1.3 and 1.4), can be substituted in the reaction to provide 1,4,5-substituted 1,2,3-triazole compounds similar to Formula 1.5.

### 2. Route II

In one aspect, 1,4,5-substituted 1,2,3-triazole compounds can be prepared as shown below.

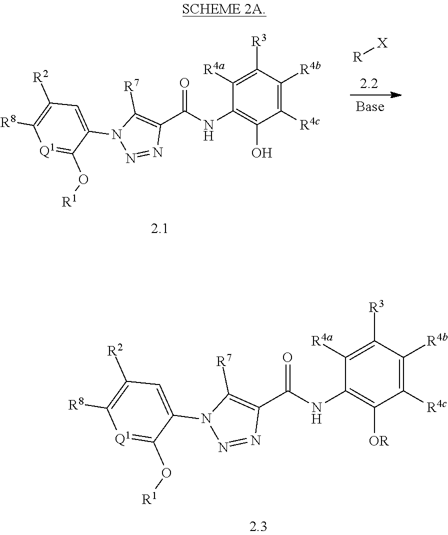

Compounds are represented in generic form, wherein X is a halogen, R is C1-C8 alkyl, and with other substituents as noted in compound descriptions elsewhere herein. As would be appreciated by one of skill in the art, a similar reaction can be used in which R^{4a}, R^{4b}, or R^{4c} is —OH, and R^{4d} is as defined elsewhere herein. A more specific example is set forth below.

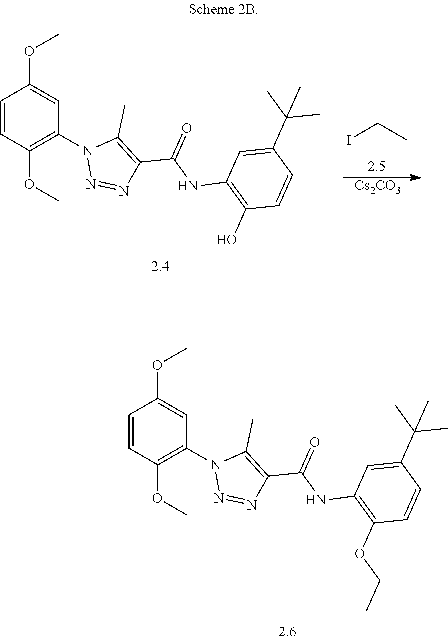

In one aspect, compounds of type 2.6, and similar compounds, can be prepared according to reaction Scheme 2B above. Thus, compounds of type 2.6 can be prepared by alkylation reaction between an appropriate phenol, e.g., 2.4 as shown above, and an appropriate aryl halide, e.g., 2.5 as shown above. Appropriate phenols and appropriate aryl halides are commercially available or prepared by methods known to one skilled in the art. The alkylation reaction is carried out in the presence of an appropriate base, e.g., cesium carbonate. As can be appreciated by one skilled in the art, the above reaction provides an example of a generalized approach wherein compounds similar in structure to the specific reactants above (compounds similar to compounds of type 2.1 and 2.2), can be substituted in the reaction to provide 1,4,5-substituted 1,2,3-triazole compounds similar to Formula 2.3.

### 3. Route III

In one aspect, 1,4,5-substituted 1,2,3-triazole compounds can be prepared as shown below.

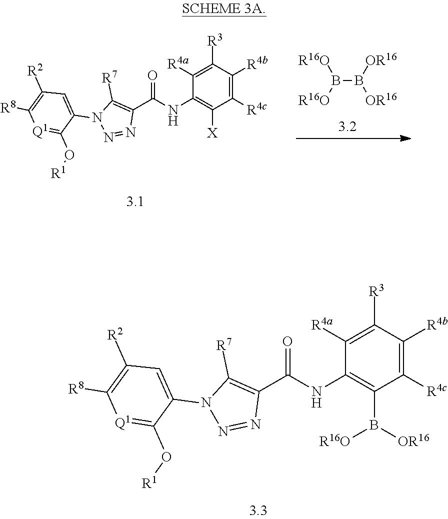

Compounds are represented in generic form, wherein X is a halogen, and with other substituents as noted in compound descriptions elsewhere herein. As would be appreciated by one of skill in the art, a similar reaction can be used in which R^{4a}, R^{4b}, or R^{4c} is —X, and R^{4d} is as defined elsewhere herein. A more specific example is set forth below.

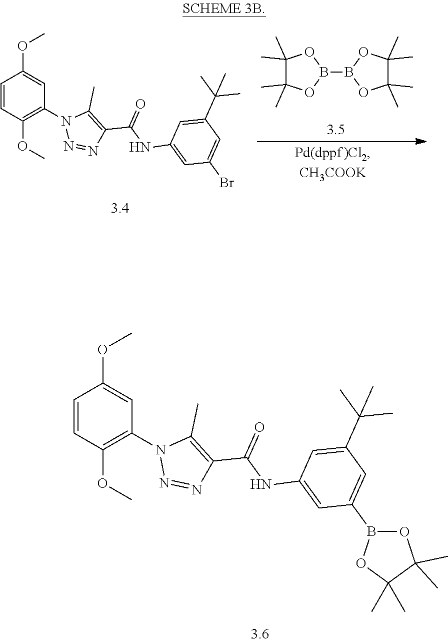

In one aspect, compounds of type 3.6, and similar compounds, can be prepared according to reaction Scheme 3B above. Thus, compounds of type 3.6 can be prepared by a coupling reaction between an appropriate aryl halide, e.g., 3.4 as shown above, and an appropriate dioxaborolane, e.g., 3.5 as shown above. Appropriate aryl halides and appropriate dioxaborolanes are commercially available or prepared by methods known to one skilled in the art. The coupling reaction is carried out in the presence of an appropriate catalyst, e.g., [1,1′-bis(diphenylphosphino)ferrocene]dichloropalladium (II), and an appropriate salt, e.g., potassium acetate. As can be appreciated by one skilled in the art, the above reaction provides an example of a generalized approach wherein compounds similar in structure to the specific reactants above (compounds similar to compounds of type 3.1 and 3.2), can be substituted in the reaction to provide 1,4,5-substituted 1,2,3-triazole compounds similar to Formula 3.3.

### 4. Route IV

In one aspect, 1,4,5-substituted 1,2,3-triazole compounds can be prepared as shown below.

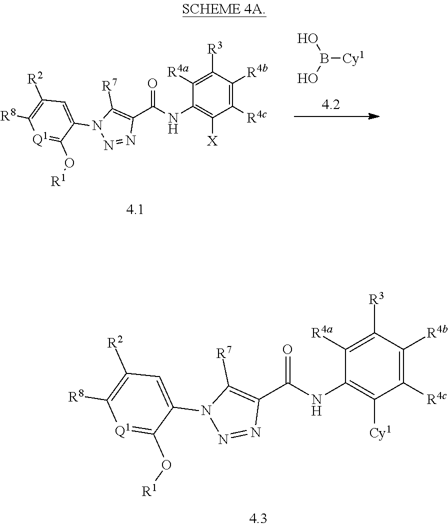

Compounds are represented in generic form, wherein X is a halogen, Cy¹ is a C2-C5 heteroaryl, and with other substituents as noted in compound descriptions elsewhere herein. As would be appreciated by one of skill in the art, a similar reaction can be used in which R^{4a}, R^{4b}, or R^{4c} is —X, and R^{4d} is as defined elsewhere herein. A more specific example is set forth below.

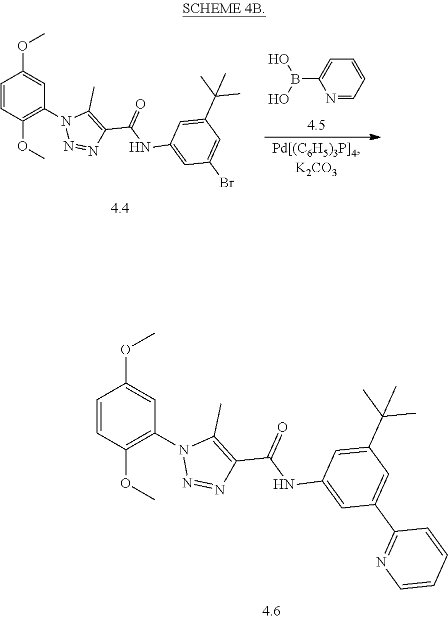 [SMILES: COc1ccc(OC)c(-n2nnc(C(=O)Nc3cc(Br)cc(C(C)(C)C)c3)c2C)c1 ; OB(O)c1ccccn1 ; COc1ccc(OC)c(-n2nnc(C(=O)Nc3cc(-c4ccccn4)cc(C(C)(C)C)c3)c2C)c1]

In one aspect, compounds of type 4.6, and similar compounds, can be prepared according to reaction Scheme 4B above. Thus, compounds of type 4.6 can be prepared by a coupling reaction between an appropriate aryl halide, e.g., 4.4 as shown above, and an appropriate aryl boronic acid, e.g., 4.5 as shown above. Appropriate aryl halides and appropriate aryl boronic acids are commercially available or prepared by methods known to one skilled in the art. The coupling reaction is carried out in the presence of an appropriate catalyst, e.g., tetrakis(triphenylphosphine)palladium(O), and an appropriate base, e.g., potassium carbonate. As can be appreciated by one skilled in the art, the above reaction provides an example of a generalized approach wherein compounds similar in structure to the specific reactants above (compounds similar to compounds of type 4.1 and 4.2), can be substituted in the reaction to provide 1,4,5-substituted 1,2,3-triazole compounds similar to Formula 4.3.

### 5. Route V

In one aspect, 1,4,5-substituted 1,2,3-triazole compounds can be prepared as shown below.

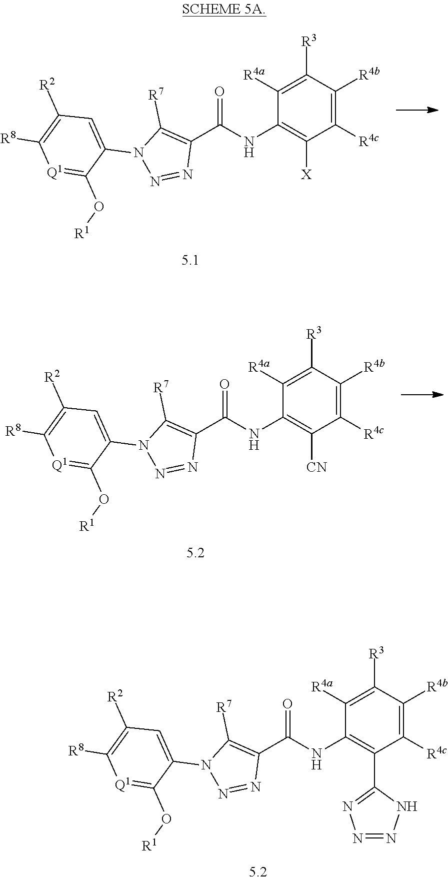

Compounds are represented in generic form, wherein X is a halogen, and with other substituents as noted in compound descriptions elsewhere herein. As would be appreciated by one of skill in the art, a similar reaction can be used in which R^{4a}, R^{4b}, or R^{4c} is —X, and R^{4d} is as defined elsewhere herein. A more specific example is set forth below.

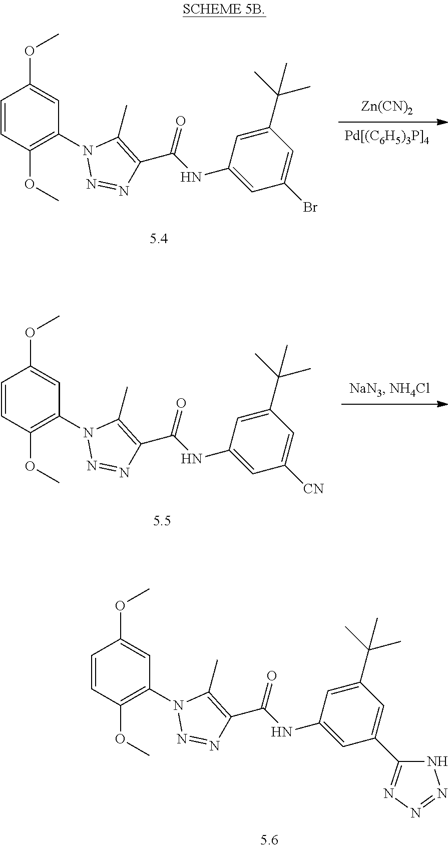

In one aspect, compounds of type 5.6, and similar compounds, can be prepared according to reaction Scheme 5B above. Thus, compounds of type 5.5 can be prepared by a nucleophilic substitution reaction of an appropriate aryl halide, e.g., 5.4 as shown above. Appropriate aryl halides are commercially available or prepared by methods known to one skilled in the art. The nucleophilic substitution reaction is carried out in the presence of an appropriate nucleophile, e.g., zinc cyanide, and an appropriate catalyst, e.g., tetrakis(triphenylphosphine)palladium(0). Compounds of type 5.6 can be prepared by cyclization of an appropriate cyanide, e.g., 5.5 as shown above. The cyclization is carried out in the presence of an appropriate azide, e.g., sodium azide, and an appropriate acidic salt, e.g., ammonium chloride. As can be appreciated by one skilled in the art, the above reaction provides an example of a generalized approach wherein compounds similar in structure to the specific reactants above (compounds similar to compounds of type 5.1 and 5.2), can be substituted in the reaction to provide 1,4,5-substituted 1,2,3-triazole compounds similar to Formula 5.3.

### 6. Route VI

In one aspect. 1,4,5-substituted 1,2,3-triazole compounds can be prepared as shown below.

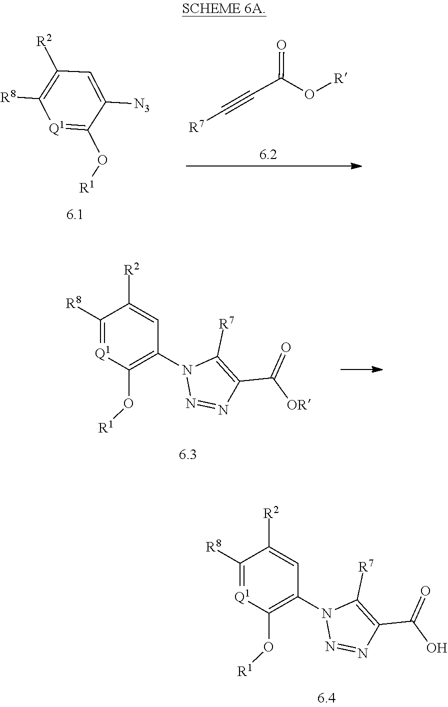

Compounds are represented in generic form, with substituents as noted in compound descriptions elsewhere herein. A more specific example is set forth below.

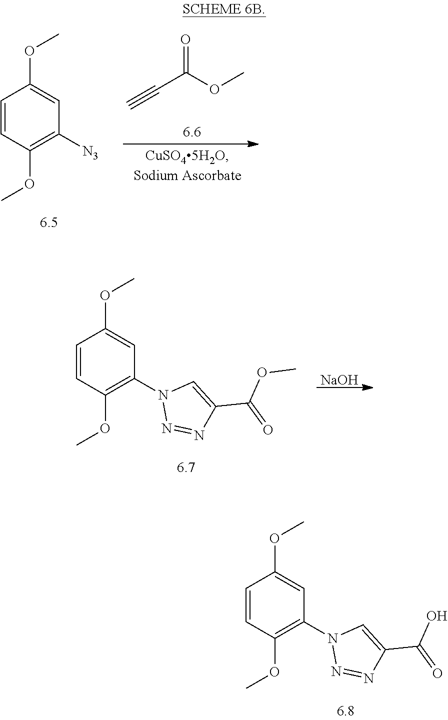

In one aspect, compounds of type 6.8, and similar compounds, can be prepared according to reaction Scheme 6B above. Thus, compounds of type 6.7 can be prepared by a cyclization of an appropriate azide, e.g., 6.5 as shown above. Appropriate azides are commercially available or prepared by methods known to one skilled in the art. The cyclization is carried out in the presence of an appropriate alkyne, e.g., 6.6 as shown above, an appropriate catalyst, e.g., copper sulfate pentahydrate, and an appropriate salt, e.g., sodium ascorbate. Appropriate alkynes are commercially available or prepared by methods known to one skilled in the art. Compounds of type 6.8 can be prepared by reduction of an appropriate alkyl ester, e.g., 6.7 as shown above. The reduction is carried out in the presence of an appropriate base, e.g., sodium hydroxide. As can be appreciated by one skilled in the art, the above reaction provides an example of a generalized approach wherein compounds similar in structure to the specific reactants above (compounds similar to compounds of type 6.1, 6.2, and 6.3), can be substituted in the reaction to provide 1,4,5-substituted 1,2,3-triazole compounds similar to Formula 6.4.

### 7. Route VII

In one aspect, 1,4,5-substituted 1,2,3-triazole compounds can be prepared as shown below.

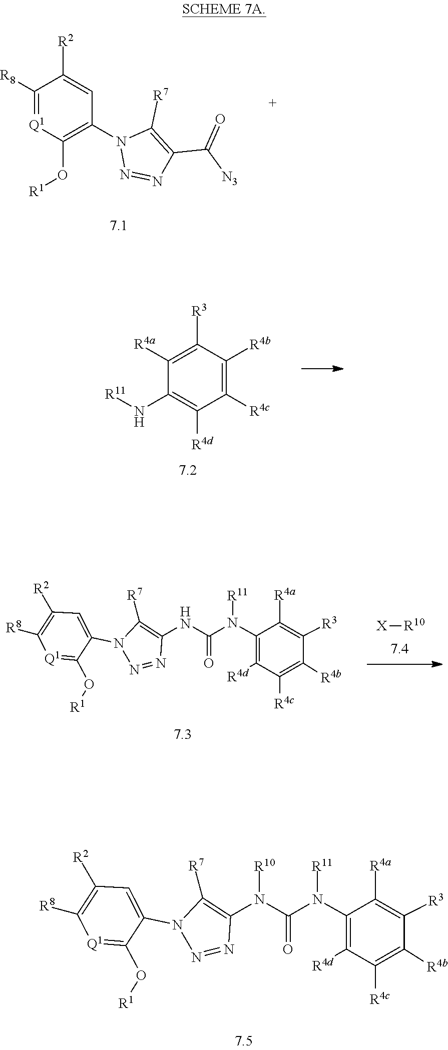

Compounds are represented in generic form, where X is halogen and with other substituents as noted in compound descriptions elsewhere herein. A more specific example is set forth below.

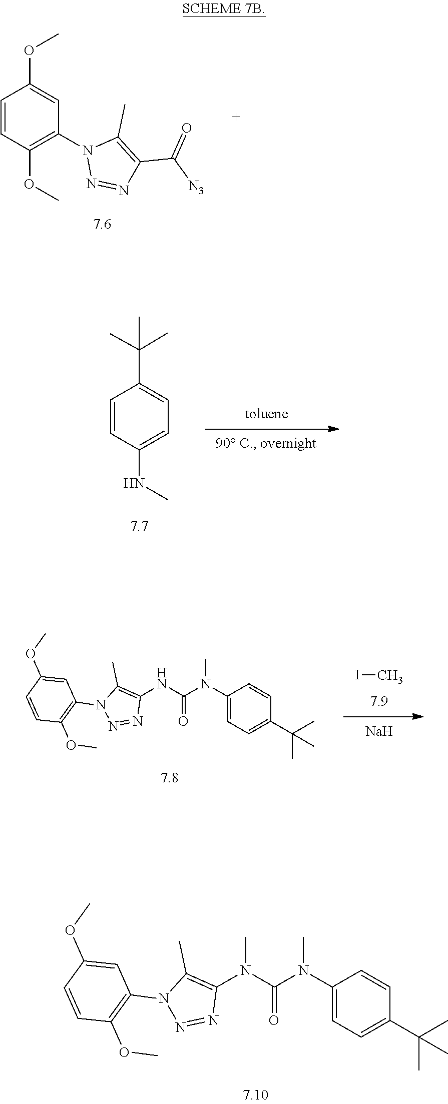

In one aspect, compounds of type 7.6, and similar compounds, can be prepared according to reaction Scheme 7B above. Thus, compounds of type 7.8 can be prepared by a addition and rearrangement of an appropriate azide, e.g., 7.6 as shown above, and an appropriate aniline, e.g., 7.7 as shown above. Appropriate azides and appropriate anilines are commercially available or prepared by methods known to one skilled in the art. The reaction is carried out in the presence of an appropriate solvent, e.g., toluene, at an appropriate temperature, e.g., 90° C. Compounds of type 7.10 can be prepared by alkylation of an appropriate urea, e.g., 7.8 as shown above. The reaction is carried out in the presence of an appropriate alkyl halide, e.g., 7.9 as shown above, and an appropriate base, e.g., sodium hydride as shown above. As can be appreciated by one skilled in the art, the above reaction provides an example of a generalized approach wherein compounds similar in structure to the specific reactants above (compounds similar to compounds of type 7.1, 7.2, 7.3, and 7.4), can be substituted in the reaction to provide 1,4,5-substituted 1,2,3-triazole compounds similar to Formula 7.5.

### 8. Route VIII

In one aspect. 1,4,5-substituted 1,2,3-triazole compounds can be prepared as shown below.

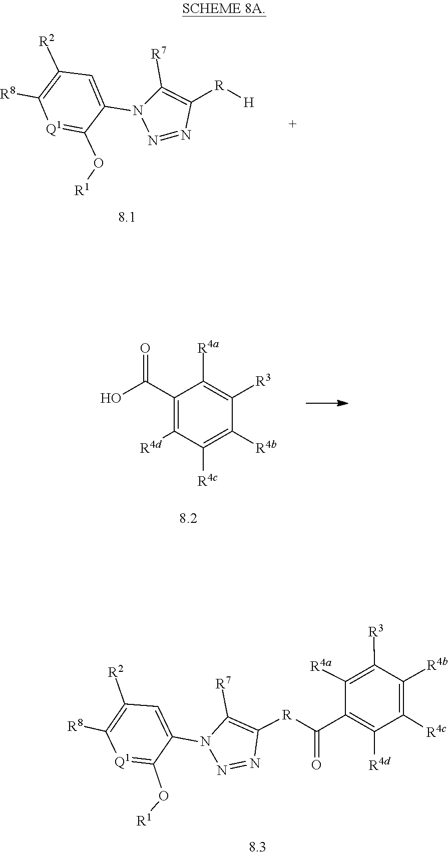

Compounds are represented in generic form, wherein R is —NR¹⁰— or —S—, and with substituents as noted in compound descriptions elsewhere herein. As would be understood by one of ordinary skill in the art, a similar reaction could also be performed in which the —CO₂H group is replaced with a —SO₂H and the —RH group is replaced by a —N(R¹⁰)H, thereby generated a —N(R¹⁰)SO₂— linker moiety. A more specific example is set forth below.

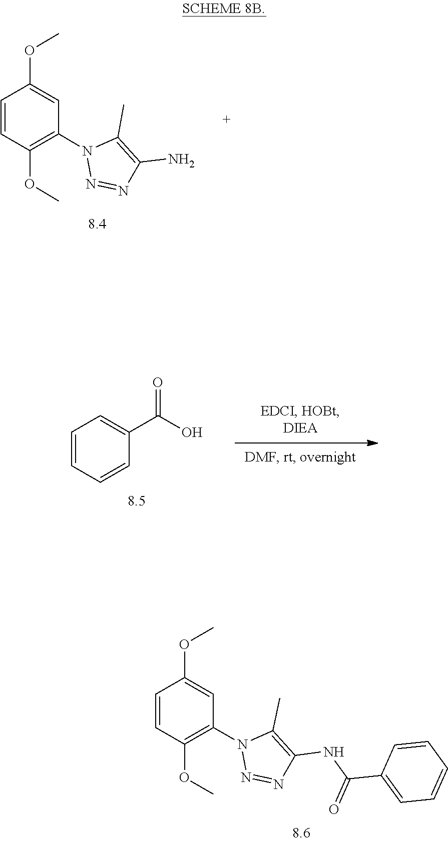 [SMILES: COc1ccc(OC)c(-n2nnc(N)c2C)c1 ; O=C(O)c1ccccc1 ; COc1ccc(OC)c(-n2nnc(NC(=O)c3ccccc3)c2C)c1]

In one aspect, compounds of type 8.6, and similar compounds, can be prepared according to reaction Scheme 8B above. Thus, compounds of type 8.6 can be prepared by a coupling reaction between an appropriate amine, e.g., 8.4 as shown above, and appropriate carboxylic acid, e.g., 8.5 as shown above. Appropriate amines and appropriate carboxylic acids are commercially available or prepared by methods known to one skilled in the art. The coupling reaction is carried out in the presence of an appropriate coupling agent, e.g., 1-ethyl-3-(3-dimethylaminopropyl)carbodiimide (EDCI), an appropriate activating agent, e.g., hydroxybenzotriazole (HOBt), and an appropriate base, e.g., N, N-diisopropylethylamine (DIEA), in an appropriate solvent, e.g., dimethylformamide (DMF). As can be appreciated by one skilled in the art, the above reaction provides an example of a generalized approach wherein compounds similar in structure to the specific reactants above (compounds similar to compounds of type 8.2 and 8.3), can be substituted in the reaction to provide 1,4,5-substituted 1,2,3-triazole compounds similar to Formula 8.4.

### D. Pharmaceutical Compositions

In one aspect, the invention relates to pharmaceutical compositions comprising the disclosed compounds and products of disclosed methods. That is, a pharmaceutical composition can be provided comprising an effective amount of at least one disclosed compound, at least one product of a disclosed method, or a pharmaceutically acceptable salt, thereof, and a pharmaceutically acceptable carrier. In an aspect, the invention relates to pharmaceutical compositions comprising a pharmaceutically acceptable carrier and an effective amount of at least one disclosed compound; or a pharmaceutically acceptable salt, thereof.

Thus, in one aspect, disclosed are pharmaceutical compositions comprising an effective amount of a disclosed compound or a pharmaceutically acceptable salt thereof, and a pharmaceutically acceptable carrier.

In a further aspect, the compound has a structure represented by a formula:

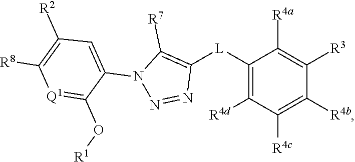

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is hydrogen, halogen, C1-C8 alkyl, C1-C8 haloalkyl, C1-C8 alkoxy, C1-C8 haloalkoxy, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

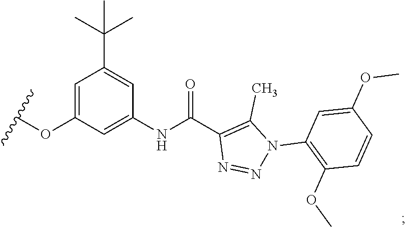

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy. C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R⁸ is selected from hydrogen, halogen, and C1-C4 alkoxy; wherein R⁷ is selected from hydrogen and C1-C4 alkyl; provided that when L is —C(O)NR¹⁰—, then R² is selected from C1-C4 haloalkyl, C1-C4 alkoxy, and C1-C4 haloalkoxy, and R³ is C1-C8 alkyl, —CO₂(C1-C4 alkyl), or —C(O)Cy², or a pharmaceutically acceptable salt thereof.

In a further aspect, the compound has a structure a structure represented by a formula:

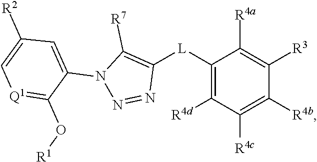

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is selected from hydrogen, halogen, C1-C8 alkyl, C1-C8 haloalkyl, C1-C8 alkoxy, and C1-C8 haloalkoxy; and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl. C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, B(OR¹⁶)₂, —OCy¹, and Cy¹; wherein n, when present, is selected from 1, 2, 3, 4, and 5; wherein R¹¹, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

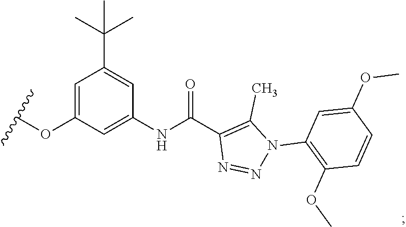

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein R⁷ is selected from hydrogen and C1-C4 alkyl, and wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl), provided that when L is —C(O)NR¹⁰—, then R² is selected from C1-C4 haloalkyl, C1-C4 alkoxy, and C1-C4 haloalkoxy, and R³ is C1-C8 alkyl, or a pharmaceutically acceptable salt thereof.

In a further aspect, the compound has a structure a structure represented by a formula:

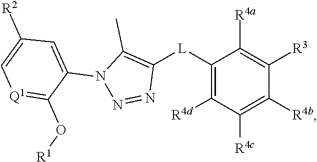

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is selected from hydrogen, halogen, C1-C8 alkyl, C1-C8 haloalkyl, C1-C8 alkoxy, and C1-C8 haloalkoxy; and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)OR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

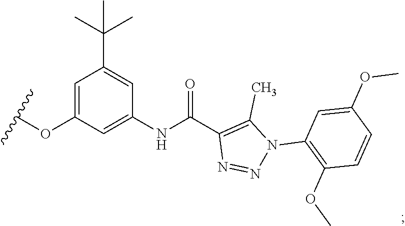

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; and wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl), provided that when L is —C(O)NR¹⁰—, then R² is selected from C1-C4 haloalkyl, C1-C4 alkoxy, and C1-C4 haloalkoxy, and R³ is C1-C8 alkyl, or a pharmaceutically acceptable salt thereof.

In a further aspect, the compound is selected from:

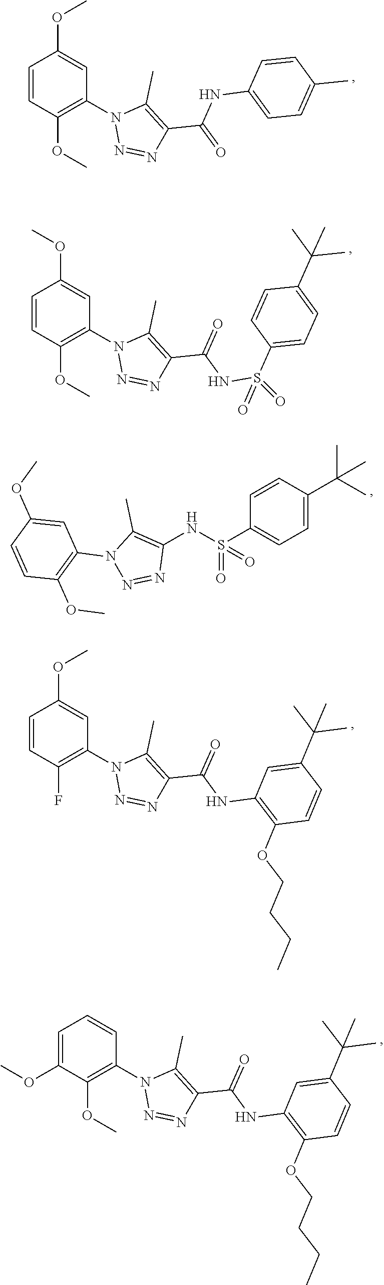

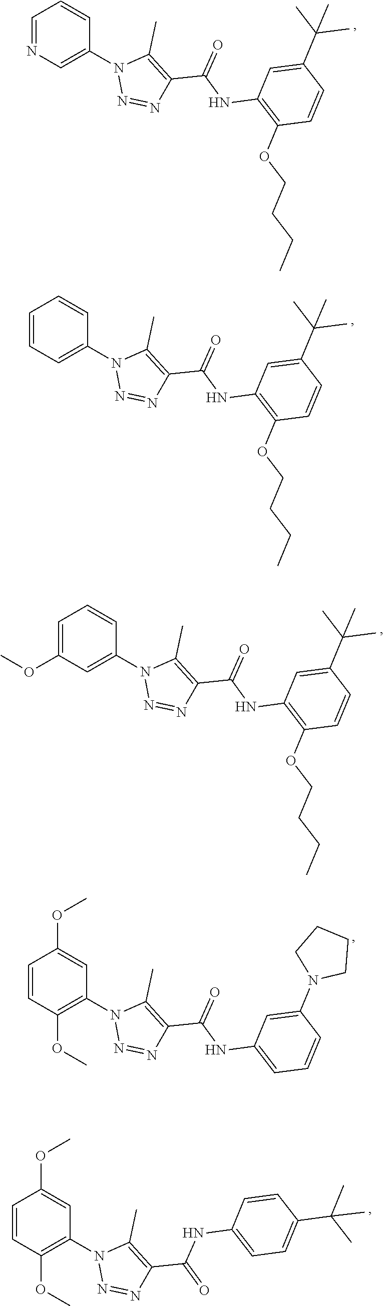

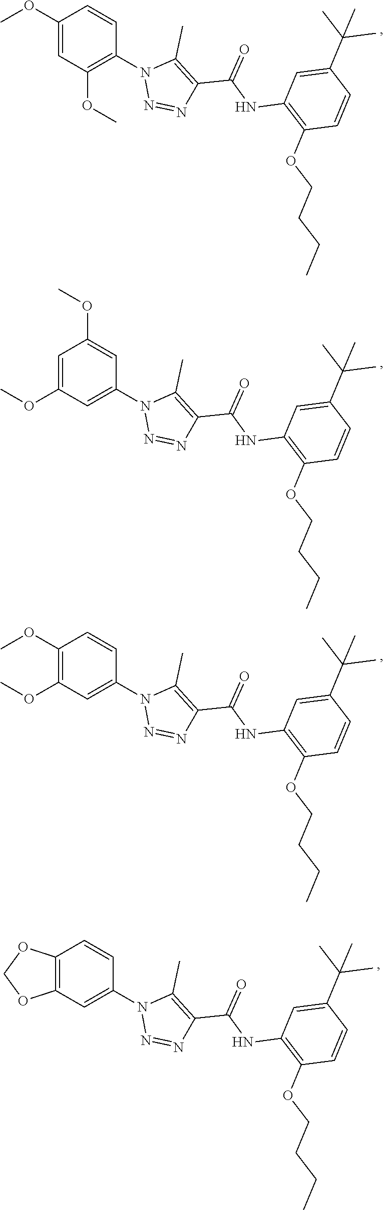

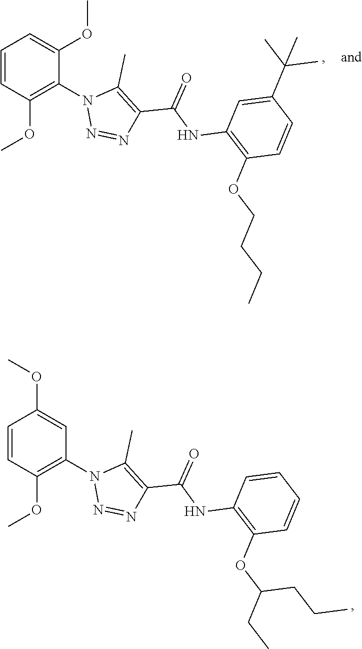 [SMILES: CCCCOc1ccc(C(C)(C)C)cc1NC(=O)c1nnn(-c2c(OC)cccc2OC)c1C ; CCCC(CC)Oc1ccccc1NC(=O)c1nnn(-c2cc(OC)ccc2OC)c1C]

or a pharmaceutically acceptable salt thereof.

In a further aspect, the compound has a structure represented by a formula:

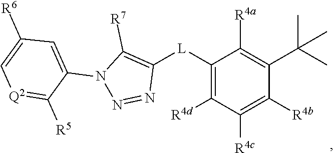

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹¹; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy, and wherein R⁷ is selected from hydrogen and Ct-C4 alkyl, or a pharmaceutically acceptable salt thereof.

In a further aspect, the compound has a structure represented by a formula:

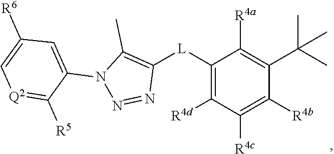

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹¹; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; and wherein R⁶ is selected from hydrogen and C1-C4 alkoxy, or a pharmaceutically acceptable salt thereof.

In a further aspect, the compound is selected from:

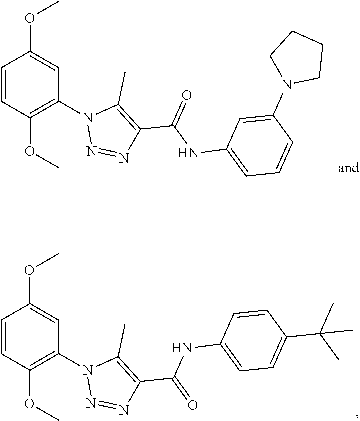 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

In a further aspect, the compound has a structure represented by a formula:

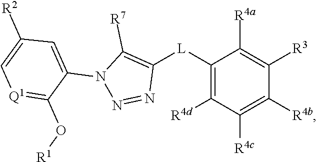

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

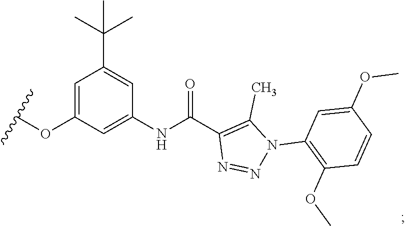

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1—C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof.

In a further aspect, the compound has a structure represented by a formula:

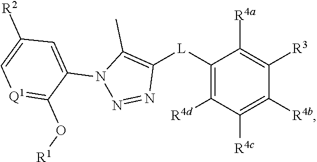

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

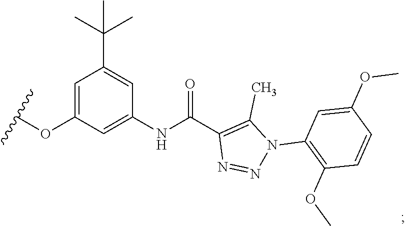

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R¹⁰ is selected from hydrogen and C1-C4 alkyl, or a pharmaceutically acceptable salt thereof.

In a further aspect, the effective amount is a therapeutically effective amount. In a still further aspect, the effective amount is a prophylactically effective amount. In a still further aspect, the pharmaceutical composition comprises a compound that is a product of a disclosed method of making.

In a further aspect, the pharmaceutical composition comprises a therapeutically effective amount of a compound having a structure represented by a formula:

In a further aspect, the pharmaceutical composition comprises a disclosed compound. In yet a further aspect, the pharmaceutical composition comprises a product of a disclosed method of making.

In a further aspect, the mammal has been diagnosed with a need for treatment of a disorder of uncontrolled cellular proliferation such as, for example, a cancer. In yet a further aspect, the mammal has been diagnosed with a need for treatment of a disorder of uncontrolled cellular proliferation prior to the administering step. In an even further aspect, the mammal has been identified to be in need of treatment of a disorder of uncontrolled cellular proliferation. In a still further aspect, a therapeutic agent known to treat a disorder of uncontrolled cellular proliferation is co-administered with the pharmaceutical composition. In yet a further aspect, the therapeutic agent known to treat a disorder of uncontrolled cellular proliferation that is co-administered with the pharmaceutical composition is paclitaxel, irinotecan, leucovorin, dasatinib, or erlotinib.

In a further aspect, the pharmaceutical composition is used to treat a mammal. In a further aspect, the mammal is a human. In a still further aspect, the mammal has been diagnosed with a need for modulating an adverse drug reaction prior to the administering step. In an even further aspect, the mammal has been identified to be in need of treatment of an adverse drug reaction. In a still further aspect, the adverse drug reaction is enhanced toxicity, increased metabolism, and/or decreased efficacy. In yet a further aspect, the adverse drug reaction is associated with another therapeutic agent, and the pharmaceutical composition is administered to treat the adverse drug reaction associated with the other therapeutic agent. In an even further aspect, the adverse drug reaction is associated with another therapeutic agent, and the pharmaceutical composition is co-administered with the other therapeutic agent in order to treat the adverse drug reaction associated with the other therapeutic agent. In a further aspect, the pharmaceutical composition is used to decrease an adverse drug reaction.

In a further aspect, the mammal has been diagnosed with a need for treatment of a bowel disorder such as, for example, irritable bowel syndrome (IBS). In yet a further aspect, the mammal has been diagnosed with a need for treatment of a bowel disorder prior to the administering step. In an even further aspect, the mammal has been identified to be in need of treatment of a bowel disorder.

In various aspects, the pharmaceutical composition of the present invention comprises a pharmaceutically acceptable carrier; an effective amount of at least one disclosed compound; or a pharmaceutically acceptable salt thereof; and an anticancer agent. In a further aspect, the anticancer agent comprises a compound selected from paclitaxel, irinotecan, leucovorin, dasatinib, and erlotinib, or combinations thereof. In a still further aspect, the anticancer agent comprises a compound selected from paclitaxel, docetaxel, vinblastine, vincristine, vinorelbine, camptothecin, topotecan, irinotecan, belotecan, gimatecan, inidimitecan, indotecan, Genz-644282, daunorubicin, epirubicin, etoposide, teniposide, mitoxantrone, ellipticinium, vasaroxin, dexrazoxane, mebarone, 3-hydroxy-2-[(1R)-6-isopropenyl-3-methyl-cyclohex-2-en-1-yl]-5-pentyl-1,4-benzoquinone (HU-331), axitinib, crizotinib, dasatinib, erlotinib, gefitinib, imatinib, lapatinib, nilotinib, pazopanib, regorafenib, ruxolitinib, sorafenib, sunitinib, vandetanib, vemurafenib, doxorubicin, mitoxantrone, bleomycin, daunorubicin, dactinomycin, epirubicin, idarubicin, plicamycin, mitomycin, pentostatin, valrubicin, gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, methotrexate coadministered with leucovorin, thioguanine, carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin, etopside, vincristine, ixabepilone, vinorelbine, vinblastine, teniposide, everolimus, siroliumus, temsirolimus, or combinations thereof.

In various aspects, the pharmaceutical composition of the present invention comprises a pharmaceutically acceptable carrier; an effective amount of at least one disclosed compound; or a pharmaceutically acceptable salt thereof; and an anticancer agent. In a further aspect, the anticancer agent comprises a compound selected from paclitaxel, irinotecan, leucovorin, dasatinib, and erlotinib, or combinations thereof. In a still further aspect, the anticancer agent comprises a compound selected from paclitaxel, docetaxel, vinblastine, vincristine, vinorelbine, camptothecin, topotecan, irinotecan, belotecan, gimatecan, inidimitecan, indotecan, Genz-644282, daunorubicin, epirubicin, etoposide, teniposide, mitoxantrone, ellipticinium, vasaroxin, dexrazoxane, mebarone, 3-hydroxy-2-[(1R)-6-isopropenyl-3-methyl-cyclohex-2-en-1-yl]-5-pentyl-1,4-benzoquinone (HU-331), axitinib, crizotinib, dasatinib, erlotinib, gefitinib, imatinib, lapatinib, nilotinib, pazopanib, regorafenib, ruxolitinib, sorafenib, sunitinib, vandetanib, vemurafenib, doxorubicin, mitoxantrone, bleomycin, daunorubicin, dactinomycin, epirubicin, idarubicin, plicamycin, mitomycin, pentostatin, valrubicin, gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, methotrexate coadministered with leucovorin, thioguanine, carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin, etopside, vincristine, ixabepilone, vinorelbine, vinblastine, teniposide, everolimus, siroliumus, temsirolimus, or combinations thereof.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids,” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require modulation of PXR activity, an appropriate dosage level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The present invention is further directed to a method for the manufacture of a medicament for modulating PXR activity (e.g., modulating adverse reactions, treating a disease of uncontrolled cellular proliferation, treating a bowel disorder) in mammals (e.g., humans) comprising combining one or more disclosed compounds, products, or compositions with a pharmaceutically acceptable carrier or diluent. Thus, in an aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### E. Methods of Modulating Pregnane X Receptor Activity in a Subject

In one aspect, disclosed are methods of modulating pregnane X receptor (PXR) activity in a subject in need thereof, the method comprising administering to the subject an effective amount of a disclosed compound or a pharmaceutically acceptable salt thereof.

Thus, in one aspect, disclosed are methods of modulating PXR activity in a subject in need thereof, the method comprising administering to the subject an effective amount of a compound having a structure represented by a formula:

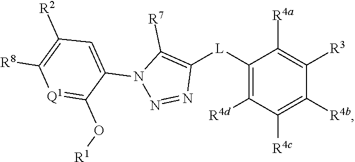

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is hydrogen, halogen, C1-C8 alkyl, C1-C8 haloalkyl, C1-C8 alkoxy, C1-C8 haloalkoxy, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2—C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

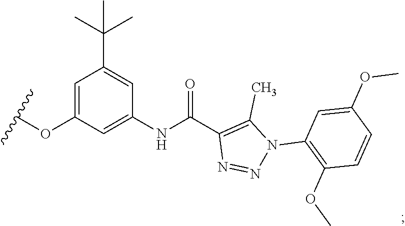

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R⁸ is selected from hydrogen, halogen, and C1-C4 alkoxy; wherein R⁷ is selected from hydrogen and C1-C4 alkyl; provided that when L is —C(O)NR¹⁰—, then R² is selected from C1-C4 haloalkyl, C1-C4 alkoxy, and C1-C4 haloalkoxy, and R³ is C1-C8 alkyl, —CO₂(C1-C4 alkyl), or —C(O)Cy², or a pharmaceutically acceptable salt thereof.

In one aspect, disclosed are methods of modulating PXR activity in a subject in need thereof, the method comprising administering to the subject an effective amount of a compound having a structure a structure represented by a formula:

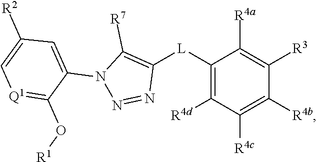

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is selected from hydrogen, halogen, C1-C8 alkyl, C1-C8 haloalkyl, C1-C8 alkoxy, and C1-C8 haloalkoxy; and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl. C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

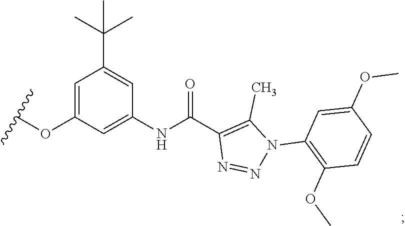

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1—C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein R⁷ is selected from hydrogen and C1-C4 alkyl, and wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl), provided that when L is —C(O)NR¹⁰—, then R² is selected from C1-C4 haloalkyl, C1-C4 alkoxy, and C1-C4 haloalkoxy, and R³ is C1-C8 alkyl, or a pharmaceutically acceptable salt thereof.

In one aspect, disclosed are methods of modulating PXR activity in a subject in need thereof, the method comprising administering to the subject an effective amount of a compound having a structure a structure represented by a formula:

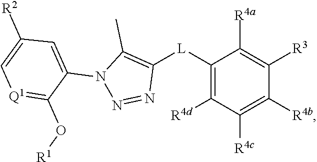

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is selected from hydrogen, halogen, C1-C8 alkyl, C1-C8 haloalkyl, C1-C8 alkoxy, and C1-C8 haloalkoxy; and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

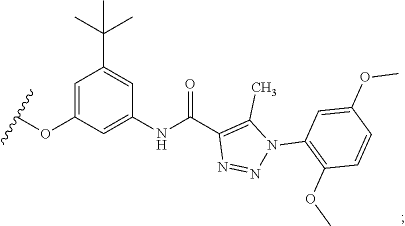

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; and wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl), provided that when L is —C(O)NR¹⁰—, then R² is selected from C1-C4 haloalkyl, C1-C4 alkoxy, and C1-C4 haloalkoxy, and R³ is C1-C8 alkyl, or a pharmaceutically acceptable salt thereof.

In a further aspect, the compound is selected from:

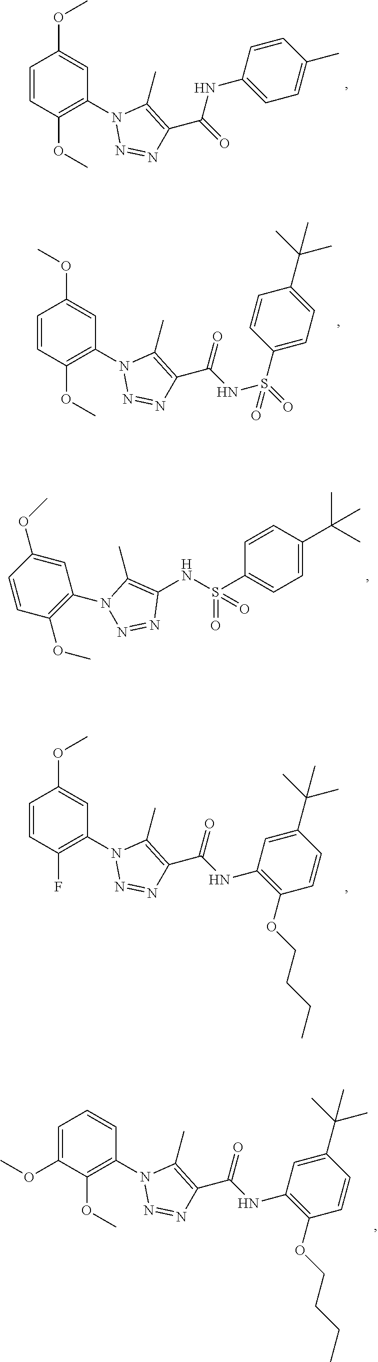

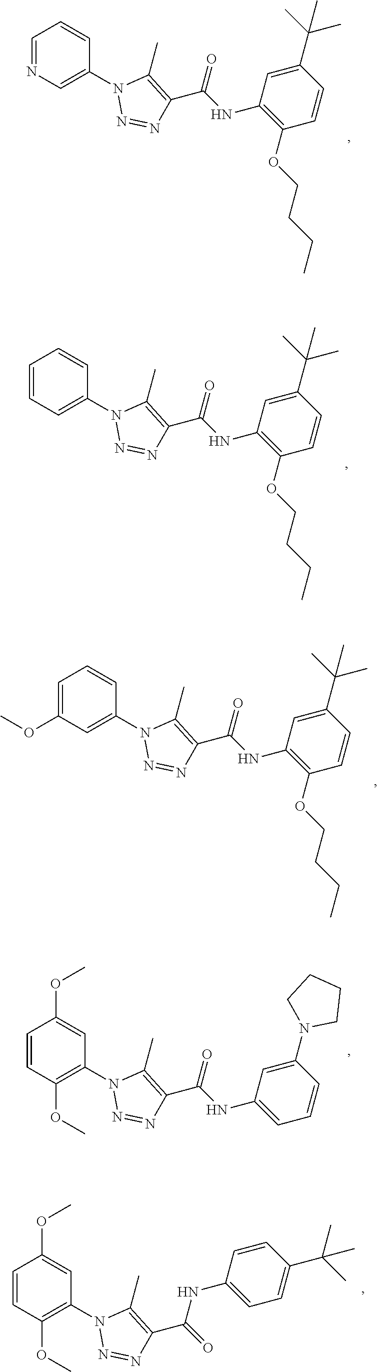

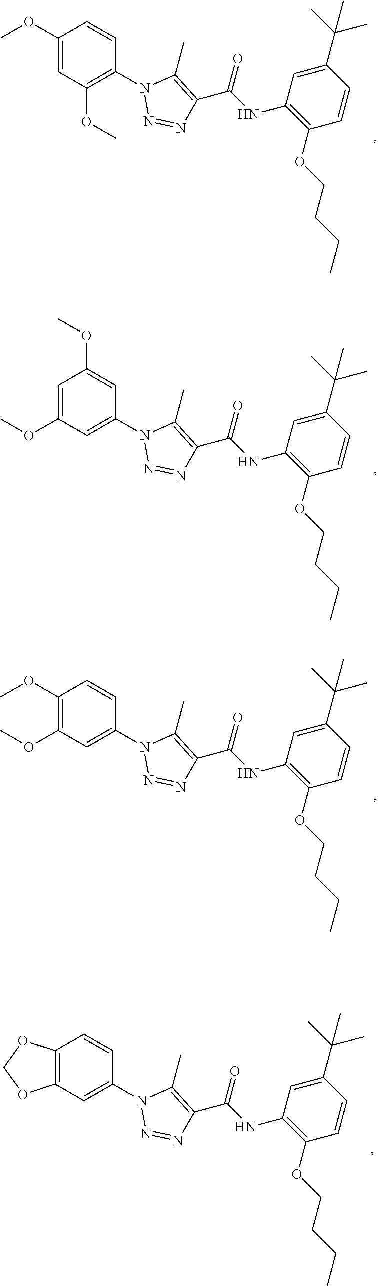

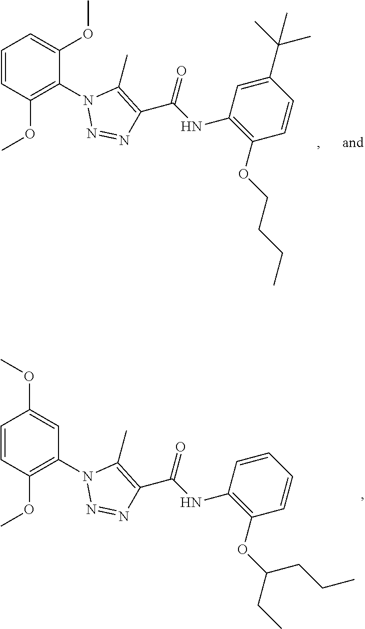 [SMILES: CCCCOc1ccc(C(C)(C)C)cc1NC(=O)c1nnn(-c2c(OC)cccc2OC)c1C ; CCCC(CC)Oc1ccccc1NC(=O)c1nnn(-c2cc(OC)ccc2OC)c1C]

or a pharmaceutically acceptable salt thereof.

In a further aspect, the compound has a structure represented by a formula:

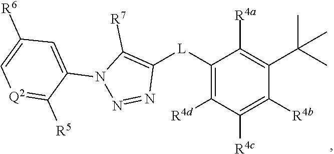

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy, and wherein R⁷ is selected from hydrogen and C1-C4 alkyl, or a pharmaceutically acceptable salt thereof.

In a further aspect, the compound has a structure represented by a formula:

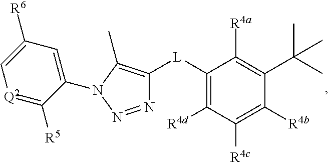

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; and wherein R⁶ is selected from hydrogen and C1-C4 alkoxy, or a pharmaceutically acceptable salt thereof.

In a further aspect, the compound is selected from:

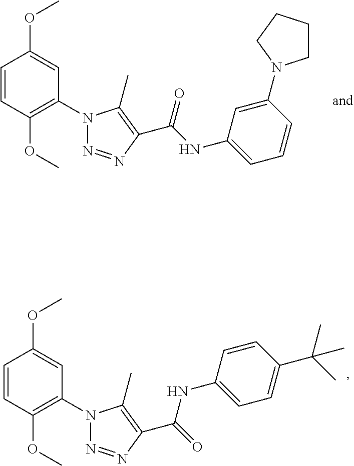 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

In a further aspect, the compound has a structure represented by a formula:

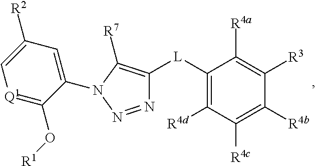

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

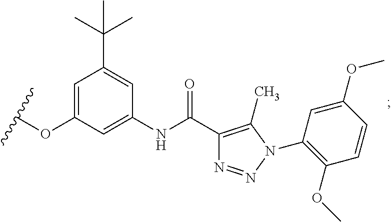

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof.

In a further aspect, the compound has a structure represented by a formula:

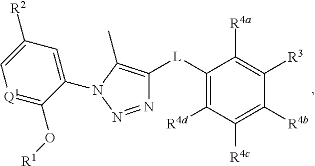

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

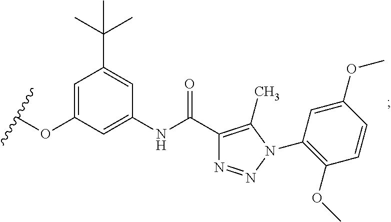

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R¹⁰ is selected from hydrogen and C1-C4 alkyl, or a pharmaceutically acceptable salt thereof.

In a further aspect, the subject is a mammal. In a still further aspect, the subject is a human.

In a further aspect, modulating is increasing. In a still further aspect, modulating is activating. In yet a further aspect, modulating is activating and the compound has a structure represented by a formula:

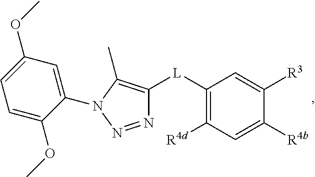

or a pharmaceutically acceptable salt thereof.

In a further aspect, modulating is decreasing. In a still further aspect, modulating is inhibiting. In yet a further aspect, modulating is inhibiting, and wherein the compound has a structure represented by a formula:

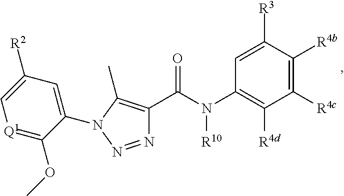

or a pharmaceutically acceptable salt thereof.

In a further aspect, the subject has been diagnosed with a need for modulating PXR activity prior to the administering step. For example, in various aspects, the subject has been diagnosed as having a disorder for which modulation of PXR activity is beneficial (e.g., cancer, a bowel disorder). In various further aspects, the subject has been diagnosed as having an adverse drug reaction for which modulation of PXR activity can be beneficial.

In a further aspect, the method further comprises identifying a subject in need for modulating PXR activity.

### F. Methods of Modulating Cellular Proliferation Activity in a Cell

In one aspect, disclosed are methods for modulating pregnane X receptor (PXR) activity in a cell, the method comprising contacting the cell with an effective amount of a disclosed compound or a pharmaceutically acceptable salt thereof.

Thus, in one aspect, disclosed are methods for modulating PXR activity in a cell, the method comprising contacting the cell with an effective amount of a compound having a structure represented by a formula:

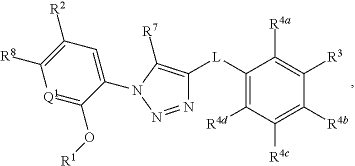

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is hydrogen, halogen, C1-C8 alkyl, C1-C8 haloalkyl, C1-C8 alkoxy, C1-C8 haloalkoxy, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

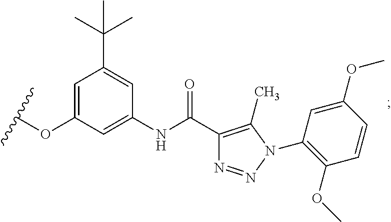

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R⁸ is selected from hydrogen, halogen, and C1-C4 alkoxy; wherein R⁷ is selected from hydrogen and C1-C4 alkyl; provided that when L is —C(O)NR¹⁰—, then R² is selected from C1-C4 haloalkyl, C1-C4 alkoxy, and C1-C4 haloalkoxy, and R³ is C1-C8 alkyl, —CO₂(C1-C4 alkyl), or C(O)Cy², or a pharmaceutically acceptable salt thereof.

In one aspect, disclosed are methods for modulating PXR activity in a cell, the method comprising contacting the cell with an effective amount of a compound having a structure a structure represented by a formula:

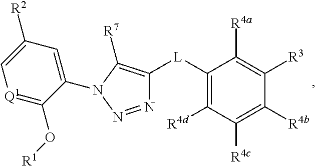

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is selected from hydrogen, halogen, C1-C8 alkyl, C1-C8 haloalkyl, C1-C8 alkoxy, and C1-C8 haloalkoxy; and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

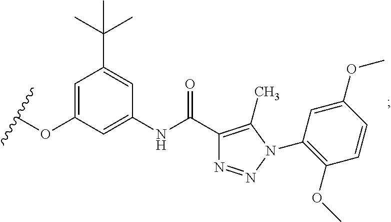

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein R⁷ is selected from hydrogen and C1-C4 alkyl, and wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl), provided that when L is —C(O)NR¹⁰—, then R² is selected from C1-C4 haloalkyl, C1-C4 alkoxy, and C1-C4 haloalkoxy, and R³ is C1-C8 alkyl, or a pharmaceutically acceptable salt thereof.

In one aspect, disclosed are methods for modulating PXR activity in a cell, the method comprising contacting the cell with an effective amount of a compound having a structure a structure represented by a formula:

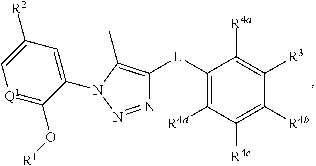

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from halogen, C1-C4 alkyl, C1-C4 haloalkyl, C1-C4 alkoxy, and C1-C4 haloalkoxy; wherein R³ is selected from hydrogen, halogen, C1-C8 alkyl, C1-C8 haloalkyl, C1-C8 alkoxy, and C1-C8 haloalkoxy; and wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

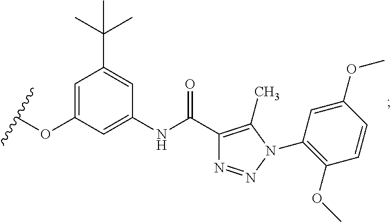

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; and wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl), provided that when L is —C(O)NR¹⁰—, then R² is selected from C1-C4 haloalkyl, C1-C4 alkoxy, and C1-C4 haloalkoxy, and R³ is C1-C8 alkyl, or a pharmaceutically acceptable salt thereof.

In a further aspect, the compound is selected from:

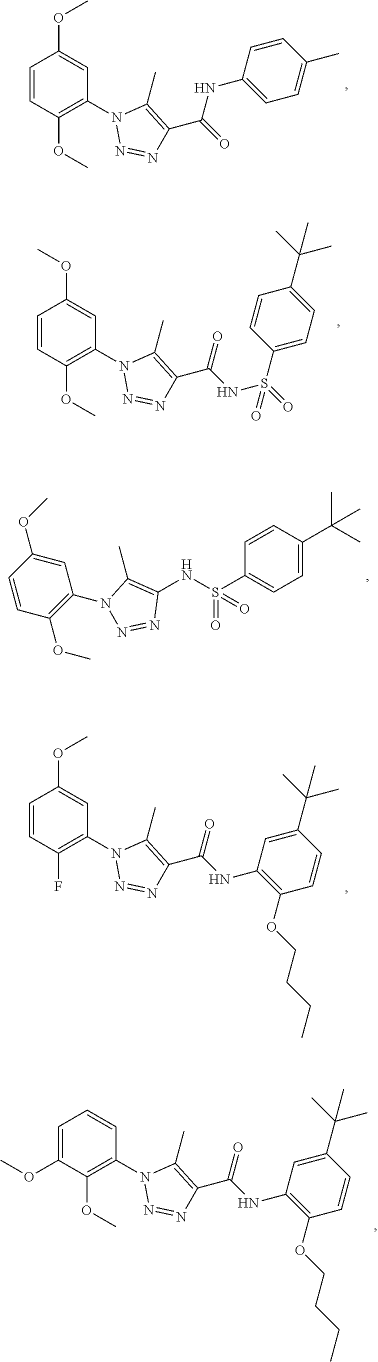

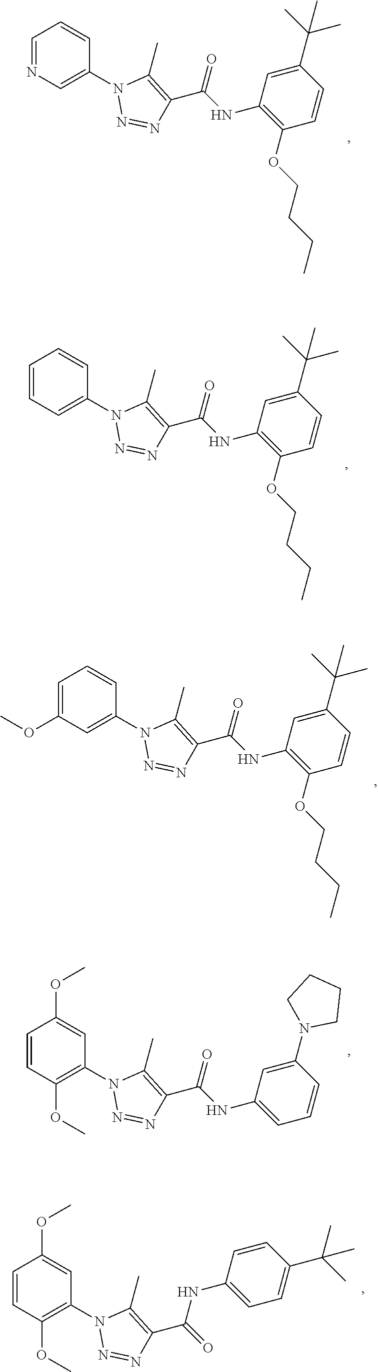

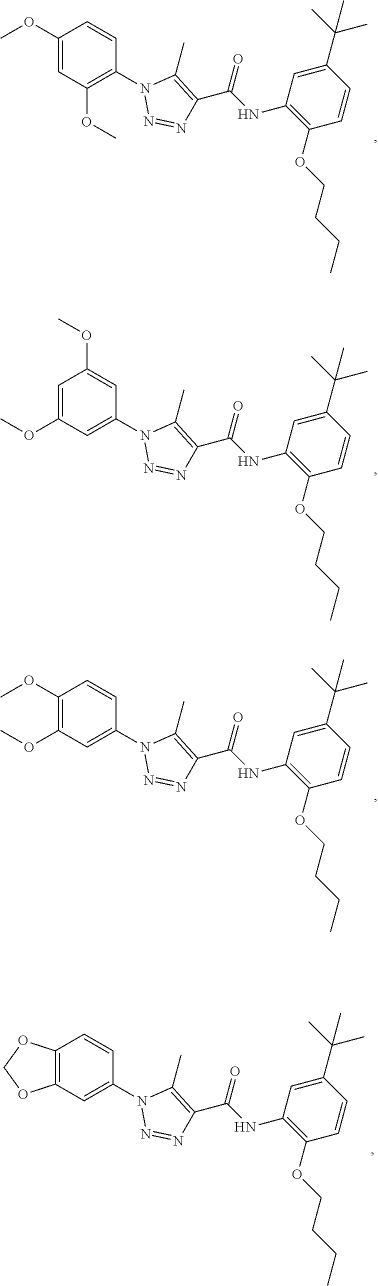

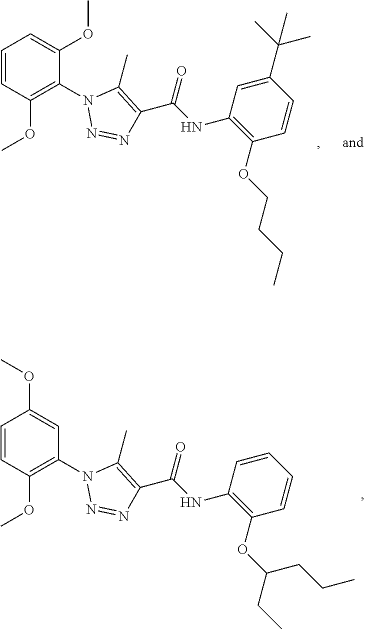 [SMILES: CCCCOc1ccc(C(C)(C)C)cc1NC(=O)c1nnn(-c2c(OC)cccc2OC)c1C ; CCCC(CC)Oc1ccccc1NC(=O)c1nnn(-c2cc(OC)ccc2OC)c1C]

or a pharmaceutically acceptable salt thereof.

In a further aspect, the compound has a structure represented by a formula:

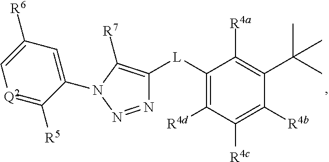

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹¹; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy, and wherein R⁷ is selected from hydrogen and C1-C4 alkyl, or a pharmaceutically acceptable salt thereof.

In a further aspect, the compound has a structure represented by a formula:

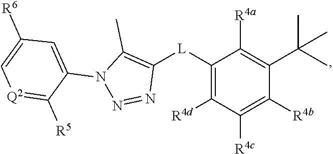

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; and wherein R⁶ is selected from hydrogen and C1-C4 alkoxy, or a pharmaceutically acceptable salt thereof.

In a further aspect, the compound is selected from:

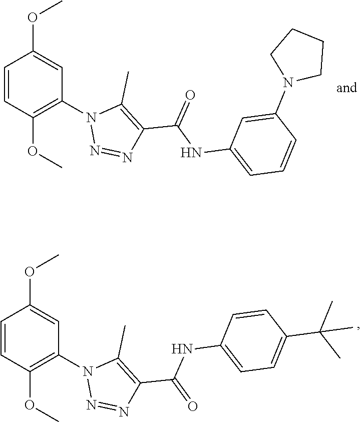 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

In a further aspect, the compound has a structure represented by a formula:

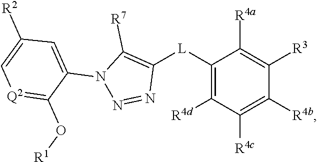

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

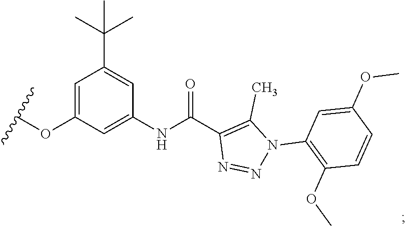

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof.

In a further aspect, the compound has a structure represented by a formula:

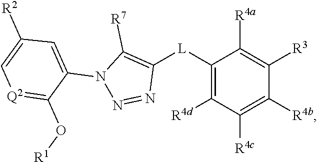

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

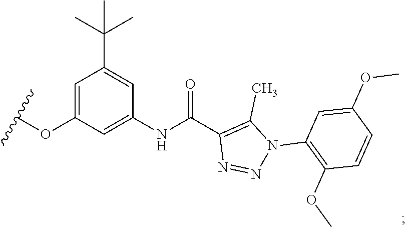

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, CONH₂, CONH(C1-C4 alkyl), and CON(C1-C4 alkyl)(C1-C4 alkyl); and wherein R¹⁰ is selected from hydrogen and C1-C4 alkyl, or a pharmaceutically acceptable salt thereof.

In a further aspect, the cell is mammalian. In a still further aspect, the cell is human. In yet a further aspect, the cell has been isolated from a mammal prior to the contacting step.

In a further aspect, contacting is ex vivo. In a still further aspect, contacting is in vitro.

In a further aspect, contacting is via administration to a mammal.

In a further aspect, the mammal has been diagnosed with a need for modulating PXR activity prior to the administering step. In a still further aspect, the mammal has been diagnosed with a need for treatment of a disorder related to PXR activity prior to the administering step.

### G. Methods of Decreasing an Adverse Drug Reaction

In various aspects, the compounds and compositions disclosed herein are useful for treating, preventing, ameliorating, controlling, reducing the risk of, or otherwise decreasing an adverse reaction associated with a drug such as, for example, an anticancer agent, an antibacterial agent, a non-steroidal anti-inflammatory agent, or an anticonvulsant agent. Thus, in one aspect, disclosed are methods for decreasing an adverse drug reaction in a subject in need thereof, the method comprising administering to the subject an effective amount of a disclosed compound or a pharmaceutically acceptable salt thereof. In a further aspect, the compound is an antagonist or an inverse agonist of pregnane X receptor activity.

In one aspect, disclosed are methods for decreasing an adverse drug reaction in a subject in need thereof, the method comprising administering to the subject an effective amount of a compound having a structure represented by a formula:

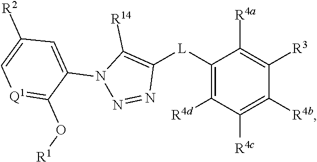

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

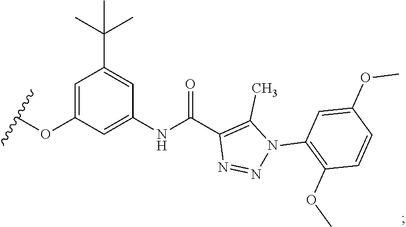

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, CONH₂, CONH(C1-C4 alkyl), and CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof.

In one aspect, disclosed are methods for decreasing an adverse drug reaction in a subject in need thereof, the method comprising administering to the subject an effective amount of a compound having a structure represented by a formula:

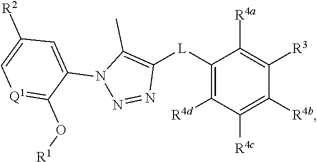

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is C1-C8 alkyl; wherein each of R^{4a}, R^{4b}, R⁴, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

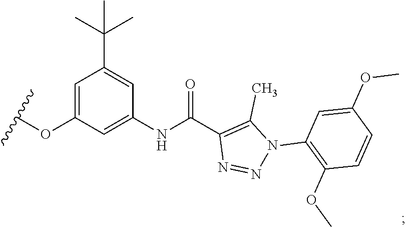

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R¹⁰ is selected from hydrogen and C1-C4 alkyl, or a pharmaceutically acceptable salt thereof.

In one aspect, disclosed are methods for decreasing an adverse drug reaction in a subject in need thereof, the method comprising administering an effective amount of a compound having a structure represented by a formula:

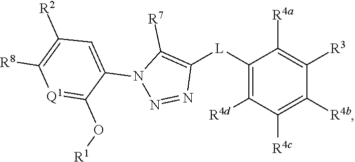

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is selected from C1-C8 alkyl, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); wherein each of R^{4a}, R^{4b}, Ra, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

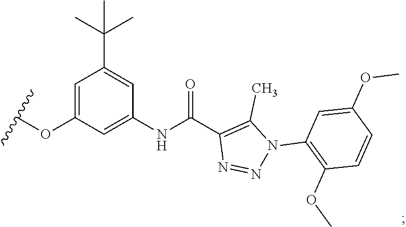

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1—C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl; wherein R⁸ is selected from hydrogen and halogen; and wherein R¹⁰ is selected from hydrogen and C1-C4 alkyl, or a pharmaceutically acceptable salt thereof.

In one aspect, disclosed are methods for decreasing an adverse drug reaction in a subject in need thereof, the method comprising administering an effective amount of a compound selected from:

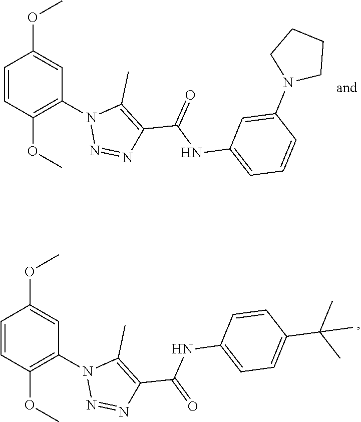 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

In one aspect, disclosed are methods of decreasing an adverse drug reaction in a subject in need thereof, the method comprising administering an effective amount of a compound selected from:

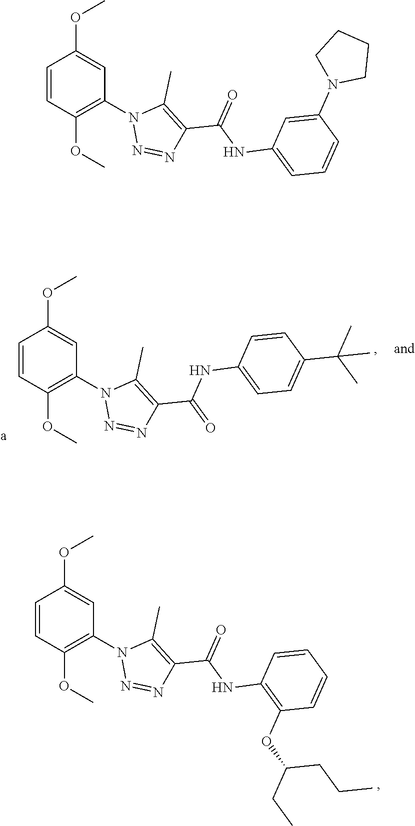 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1 ; CCC[C@H](CC)Oc1ccccc1NC(=O)c1nnn(-c2cc(OC)ccc2OC)c1C]

or a pharmaceutically acceptable salt thereof.

In various aspects, the compound is selected from:

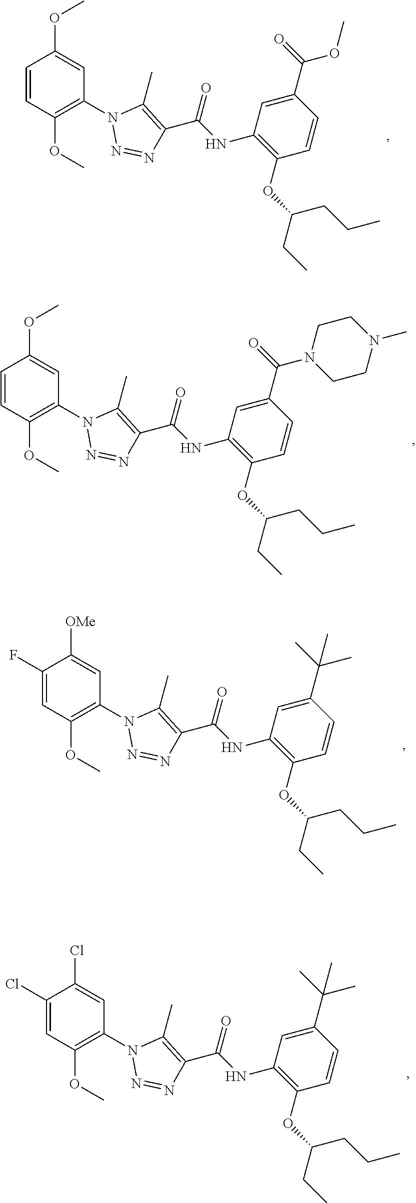 [SMILES: CCC[C@H](CC)Oc1ccc(C(=O)OC)cc1NC(=O)c1nnn(-c2cc(OC)ccc2OC)c1C ; CCC[C@H](CC)Oc1ccc(C(=O)N2CCN(C)CC2)cc1NC(=O)c1nnn(-c2cc(OC)ccc2OC)c1C ; CCC[C@H](CC)Oc1ccc(C(C)(C)C)cc1NC(=O)c1nnn(-c2cc(OC)c(F)cc2OC)c1C ; CCC[C@H](CC)Oc1ccc(C(C)(C)C)cc1NC(=O)c1nnn(-c2cc(Cl)c(Cl)cc2OC)c1C]

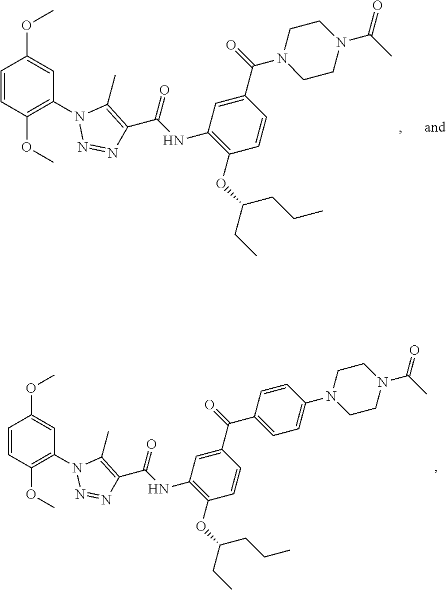 [SMILES: CCC[C@H](CC)Oc1ccc(C(=O)N2CCN(C(C)=O)CC2)cc1NC(=O)c1nnn(-c2cc(OC)ccc2OC)c1C ; CCC[C@H](CC)Oc1ccc(C(=O)c2ccc(N3CCN(C(C)=O)CC3)cc2)cc1NC(=O)c1nnn(-c2cc(OC)ccc2OC)c1C]

or a pharmaceutically acceptable salt thereof.

In various aspects, the compound is not:

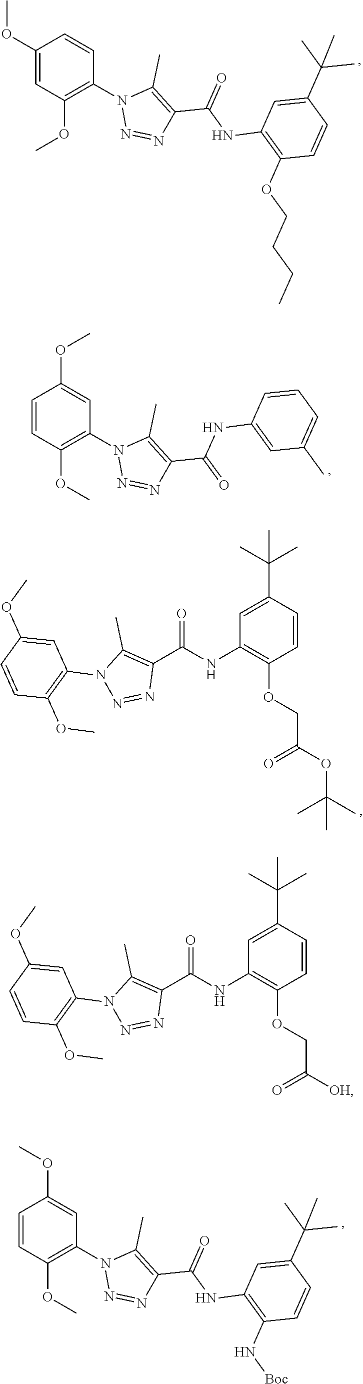 [SMILES: CCCCOc1ccc(C(C)(C)C)cc1NC(=O)c1nnn(-c2ccc(OC)cc2OC)c1C ; COc1ccc(OC)c(-n2nnc(C(=O)Nc3cccc(C)c3)c2C)c1 ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1 ; COc1ccc(OC)c(-n2nnc(C(=O)Nc3cc(C(C)(C)C)ccc3NC(=O)OC(C)(C)C)c2C)c1]

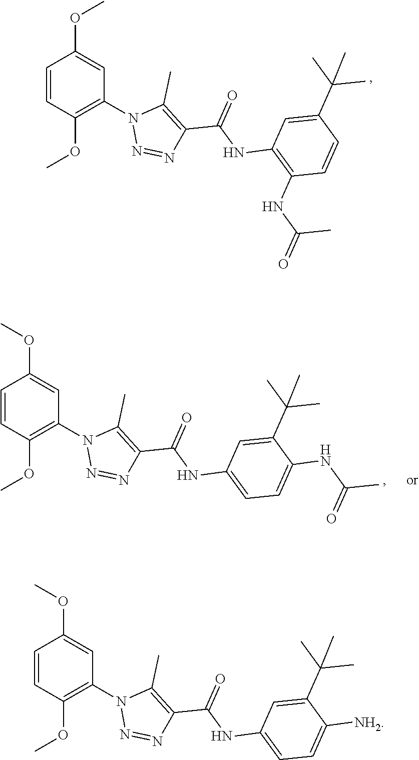 [SMILES: COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1]

In a further aspect, the subject is a mammal. In a still further aspect, the mammal is human.

In a further aspect, the subject has been diagnosed with a need for modulating an adverse drug reaction prior to the administering step. In a still further aspect, the subject is at risk for developing an adverse drug reaction prior to the administering step.

In a further aspect, the method further comprises identifying a subject in need of decreasing an adverse drug reaction.

In a further aspect, decreasing an adverse drug reaction is associated with the subject receiving treatment for a disorder of uncontrolled cellular proliferation.

In a further aspect, the method further comprises administering an anticancer agent. Examples of anticancer agents include, but are not limited to, paclitaxel, irinotecan, leucovorin, dasatinib, and erlotinib. In a still further aspect, the compound and the anticancer agent are administered simultaneously. In yet a further aspect, the compound and the anticancer agent are administered sequentially.

In a further aspect, the anticancer agent is a topoisomerase inhibitor. Examples of topoisomerase inhibitors include, but are not limited to, camptothecin, topotecan, irinotecan, belotecan, gimatecan, inidimitecan, indotecan, Genz-644282, daunorubicin, epirubicin, etoposide, teniposide, mitoxantrone, ellipticinium, vasaroxin, dexrazoxane, mebarone, and 3-hydroxy-2-[(1R)-6-isopropenyl-3-methyl-cyclohex-2-en-1-yl]-5-pentyl-1,4-benzoquinone (HU-331). In a still further aspect, the topoisomerase inhibitor is irinotecan.

In a further aspect, the anticancer agent is a tyrosine kinase inhibitor. Examples of tyrosine kinase inhibitors include, but are not limited to, axitinib, crizotinib, dasatinib, erlotinib, gefitinib, imatinib, lapatinib, nilotinib, pazopanib, regorafenib, ruxolitinib, sorafenib, sunitinib, vandetanib, and vemurafenib. In a still further aspect, the tyrosine kinase inhibitor is dasatinib or erlotinib.

In a further aspect, the anticancer agent is a mitotic inhibitor. Examples of mitotic inhibitors include, but are not limited to, paclitaxel, docetaxel, vinblastine, vincristine, and vinorelbine. In a still further aspect, the mitotic inhibitor is selected from paclitaxel and docetaxel. In yet a further aspect, the mitotic inhibitor is paclitaxel.

In a further aspect, the anticancer agent is a chemotherapeutic agent. In a still further aspect, the chemotherapeutic agent is selected from an alkylating or alkylating-like agent (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin, or a pharmaceutically acceptable salt thereof), an antimetabolite agent (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, or a pharmaceutically acceptable salt thereof), an antineoplastic antibiotic agent (e.g., doxorubicin, mitoxantrone, bleomycin, daunorubicin, dactinomycin, epirubicin, idarubicin, plicamycin, mitomycin, pentostatin, valrubicin, or a pharmaceutically acceptable salt thereof), a mitotic inhibitor agent (e.g., etopside, vincristine, ixabepilone, vinorelbine, vinblastine, teniposide, or a pharmaceutically acceptable salt thereof), and an mTor inhibitor agent (e.g., everolimus, siroliumus, temsirolimus, or a pharmaceutically acceptable salt thereof).

In a further aspect, the method further comprises administering an antibacterial agent. Examples of antibacterial agents include, but are not limited to, isoniazid, rifampicin, and flucloxacillin, or a combination thereof. In a still further aspect, the compound and the antibacterial agent are administered simultaneously. In yet a further aspect, the compound and the antibacterial agent are administered sequentially.

In a further aspect, the method further comprises administering a non-steroidal anti-inflammatory agent. Examples of non-steroidal anti-inflammatory agents include, but are not limited to, acetaminophen. In a still further aspect, the compound and the non-steroidal anti-inflammatory agent are administered simultaneously. In yet a further aspect, the compound and the non-steroidal anti-inflammatory agent are administered sequentially.

In a further aspect, the method further comprises administering an anticonvulsant agent. Examples of anticonvulsant agents include, but are not limited to, phenytoin. In a still further aspect, the compound and the anticonvulsant agent are administered simultaneously. In yet a further aspect, the compound and the anticonvulsant agent are administered sequentially.

### H. Methods of Treating a Disorder of Uncontrolled Cellular Proliferation in a Subject

In various aspects, the compounds and compositions disclosed herein are useful for treating, preventing, ameliorating, controlling, or reducing the risk of a variety of disorders associated with uncontrolled cellular proliferation, such as, for example cancer. Thus, in one aspect, disclosed are methods of treating a disorder associated with uncontrolled cellular proliferation activity in a subject, the method comprising administering to the subject an effective amount of a disclosed compound or a pharmaceutically acceptable salt thereof. In a further aspect, the compound is an antagonist or an inverse agonist of pregnane X receptor activity.

In one aspect, disclosed are methods of treating a disorder associated with uncontrolled cellular proliferation activity in a subject, the method comprising administering to the subject an effective amount of a compound having a structure represented by a formula:

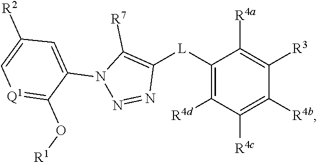

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

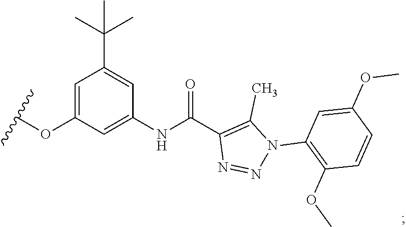

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, CONH₂, CONH(C1-C4 alkyl), and CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof.

In one aspect, disclosed are methods of treating a disorder associated with uncontrolled cellular proliferation activity in a subject, the method comprising administering to the subject an effective amount of a compound having a structure represented by a formula:

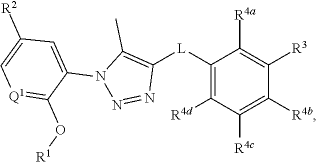

wherein Q¹ is selected from N and CH; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1—C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

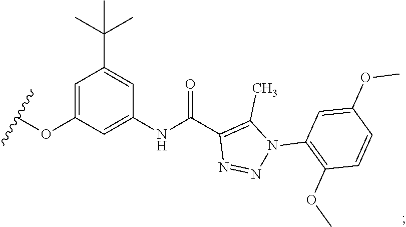

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R¹⁰ is selected from hydrogen and C1-C4 alkyl, or a pharmaceutically acceptable salt thereof.

In one aspect, disclosed are methods for treating a disorder of uncontrolled cellular proliferation in a subject in need thereof, the method comprising administering to the subject an effective amount of a compound having a structure represented by a formula:

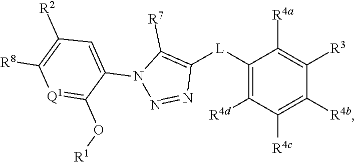

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is selected from C1-C8 alkyl, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); wherein each of R^{4a}, R^{4b}, Ra, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

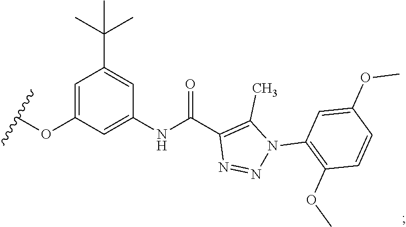

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1—C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl; wherein R⁸ is selected from hydrogen and halogen; and wherein R¹⁰ is selected from hydrogen and C1-C4 alkyl, or a pharmaceutically acceptable salt thereof.

In one aspect, disclosed are methods for treating a disorder of uncontrolled cellular proliferation in a subject in need thereof, the method comprising administering to the subject an effective amount of a compound selected from:

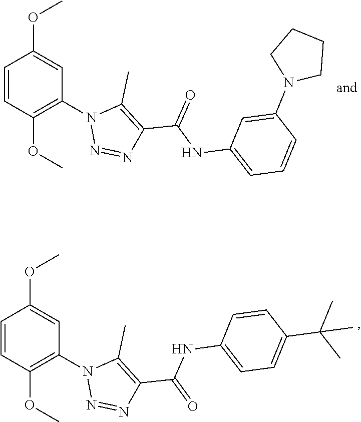 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

In one aspect, disclosed are methods for treating a disorder of uncontrolled cellular proliferation in a subject in need thereof, the method comprising administering to the subject an effective amount of a compound selected from:

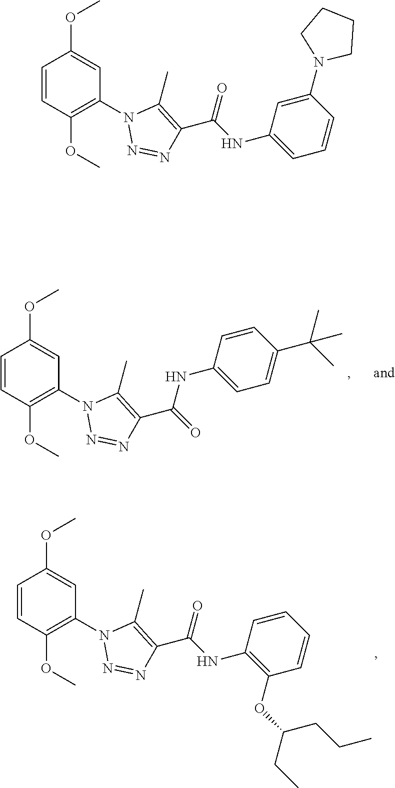 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1 ; CCC[C@H](CC)Oc1ccccc1NC(=O)c1nnn(-c2cc(OC)ccc2OC)c1C]

or a pharmaceutically acceptable salt thereof.

In one aspect, the the compound is selected from:

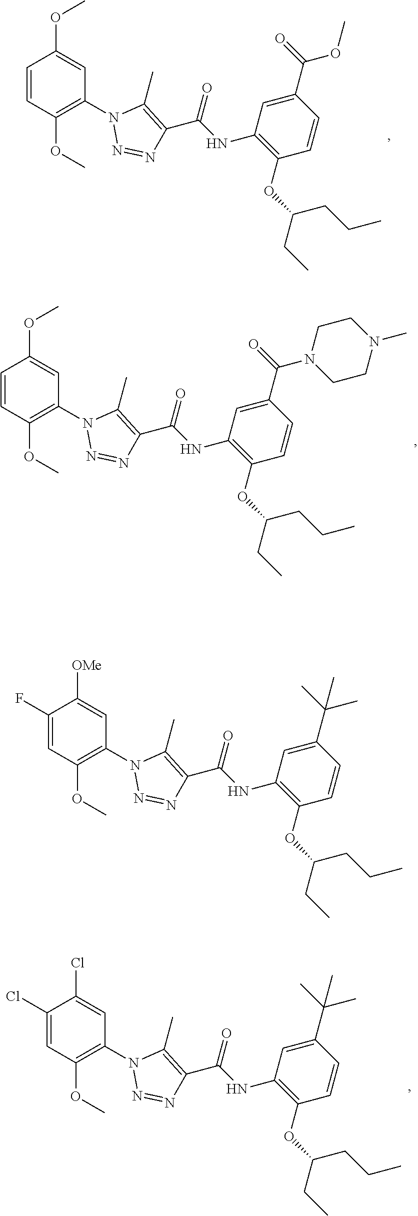 [SMILES: CCC[C@H](CC)Oc1ccc(C(=O)OC)cc1NC(=O)c1nnn(-c2cc(OC)ccc2OC)c1C ; CCC[C@H](CC)Oc1ccc(C(=O)N2CCN(C)CC2)cc1NC(=O)c1nnn(-c2cc(OC)ccc2OC)c1C ; CCC[C@H](CC)Oc1ccc(C(C)(C)C)cc1NC(=O)c1nnn(-c2cc(OC)c(F)cc2OC)c1C ; CCC[C@H](CC)Oc1ccc(C(C)(C)C)cc1NC(=O)c1nnn(-c2cc(Cl)c(Cl)cc2OC)c1C]

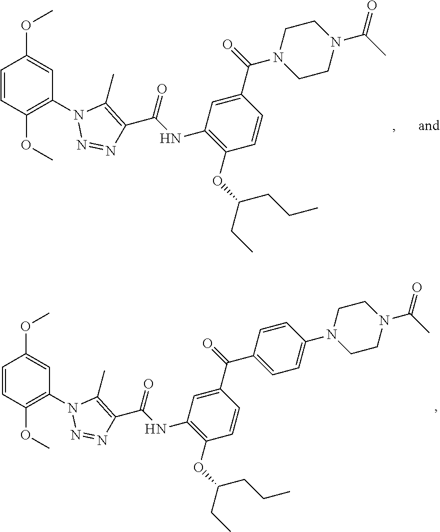 [SMILES: CCC[C@H](CC)Oc1ccc(C(=O)N2CCN(C(C)=O)CC2)cc1NC(=O)c1nnn(-c2cc(OC)ccc2OC)c1C ; CCC[C@H](CC)Oc1ccc(C(=O)c2ccc(N3CCN(C(C)=O)CC3)cc2)cc1NC(=O)c1nnn(-c2cc(OC)ccc2OC)c1C]

or a pharmaceutically acceptable salt thereof.

In various aspects, the compound is not:

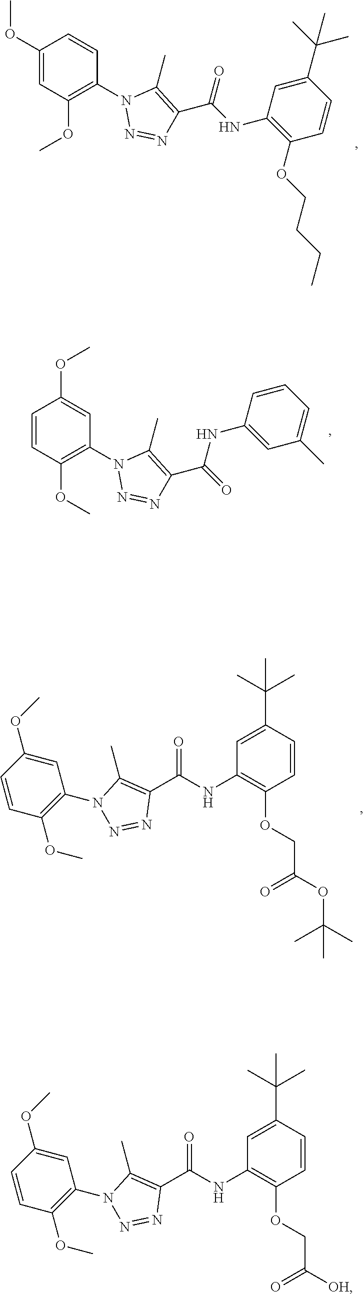 [SMILES: CCCCOc1ccc(C(C)(C)C)cc1NC(=O)c1nnn(-c2ccc(OC)cc2OC)c1C ; COc1ccc(OC)c(-n2nnc(C(=O)Nc3cccc(C)c3)c2C)c1 ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1]

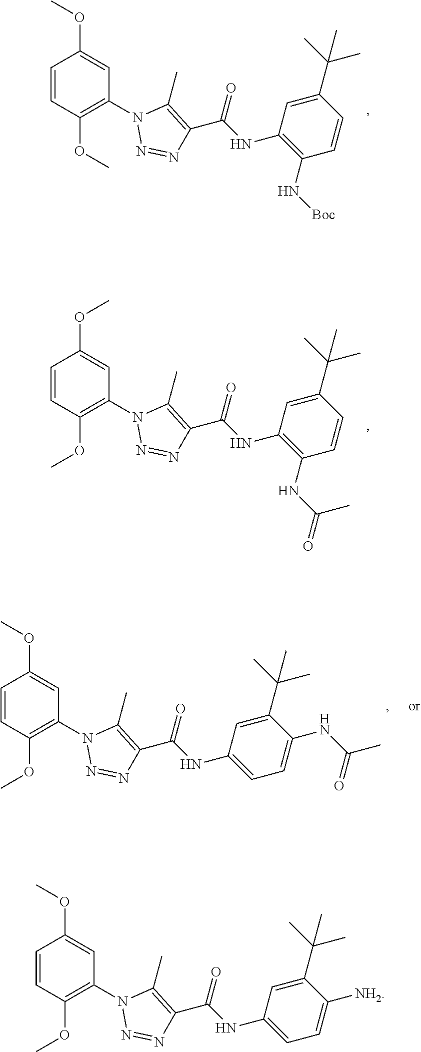 [SMILES: COc1ccc(OC)c(-n2nnc(C(=O)Nc3cc(C(C)(C)C)ccc3NC(=O)OC(C)(C)C)c2C)c1 ; COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1]

or a pharmaceutically acceptable salt thereof.

In various aspects, the disclosed compounds can be used in combination with one or more other drugs in the treatment, prevention, control, amelioration, or reduction of risk of disorders associated with uncontrolled cellular proliferation, such as for example cancer, sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), in another example acute lymphoblastic leukemia, activity for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly. Accordingly, the pharmaceutical compositions include those that contain one or more other active ingredients, in addition to a compound of the present invention.

In one aspect, the one or more other active ingredients is a chemotherapeutic agent. The chemotherapeutic agent can be selected from an alkylating agent, an antimetabolite agent, an antineoplastic antibiotic agent, a mitotic inhibitor agent, and a mTor inhibitor agent. In one aspect, the one or more other active ingredients is an antineoplastic antibiotic agent selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof. In a further aspect, the one or more other active ingredients is an antimetabolite agent selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof. In a further aspect, the one or more other active ingredients is an alkylating agent selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof. In a further aspect, the one or more other active ingredients is a mitotic inhibitor agent selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof. In a further aspect, the one or more other active ingredients is an mTor inhibitor agent selected from everolimus, siroliumus, and temsirolimus, or a pharmaceutically acceptable salt, hydrate, solvate, or polymorph thereof.

In a further aspect, the subject is a mammal. In a still further aspect, the mammal is human.

In a further aspect, the subject has been diagnosed with a need for treatment of the disorder prior to the administering step. In a still further aspect, the subject is at risk for developing the disorder prior to the administering step.

In a further aspect, the method further comprises identifying a subject at risk for developing the disorder prior to the administering step.

In a further aspect, the disorder associated with uncontrolled cellular proliferation is cancer. In a still further aspect, the cancer is selected from breast cancer, renal cancer, gastric cancer, and colorectal cancer. In yet a further aspect, the cancer is selected from lymphoma, cancers of the brain, genitourinary tract cancer, lymphatic system cancer, stomach cancer, larynx cancer, lung, pancreatic cancer, breast cancer, and malignant melanoma. In an even further aspect, the cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, plasma cell neoplasm (myeloma), and acute lymphoblastic leukemia, activity.

### I. Methods of Treating a Bowel Disorder

In various aspects, the compounds and compositions disclosed herein are useful for treating, preventing, ameliorating, controlling, or reducing the risk of a variety of bowel disorders, such as, for example, irritable bowel syndrome (IBS), Crohn's disease, celiac disease, and intestinal obstruction. Thus, in one aspect, disclosed are methods of treating a bowel disorder in a subject, the method comprising administering to the subject an effective amount of a disclosed compound or a pharmaceutically acceptable salt thereof. In a further aspect, the compound is an agonist of pregnane X receptor activity.

In one aspect, disclosed are methods of treating a bowel disorder in a subject, the method comprising administering to the subject an effective amount of a compound having a structure represented by a formula:

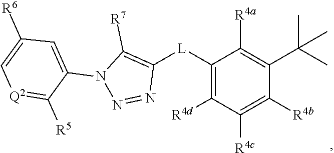

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁸ is selected from hydrogen, halogen, and C1-C4 alkoxy; wherein R⁶ is selected from hydrogen and C1-C4 alkoxy; and wherein R⁷ is selected from hydrogen and C1-C4 alkyl, or a pharmaceutically acceptable salt thereof.

In one aspect, disclosed are methods of treating a bowel disorder in a subject, the method comprising administering to the subject an effective amount of a compound having a structure represented by a formula:

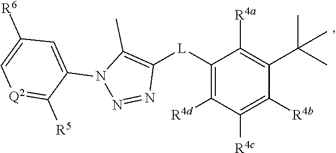

wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; and wherein R⁶ is selected from hydrogen and C1-C4 alkoxy, or a pharmaceutically acceptable salt thereof.

In one aspect, disclosed are methods of treating a bowel disorder in a subject in need thereof, the method comprising administering to the subject an effective amount of a compound having a structure represented by a formula:

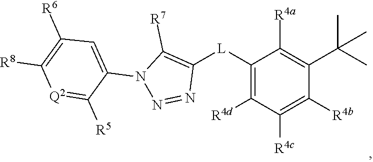

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q² is selected from N and CR¹²; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹³, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy; wherein R⁷ is selected from hydrogen and C1-C4 alkyl; and wherein R⁸ is selected from hydrogen and C1-C4 alkoxy, or a pharmaceutically acceptable salt thereof.

In one aspect, disclosed are methods of treating a bowel disorder in a subject, the method comprising administering to the subject an effective amount of a compound selected from:

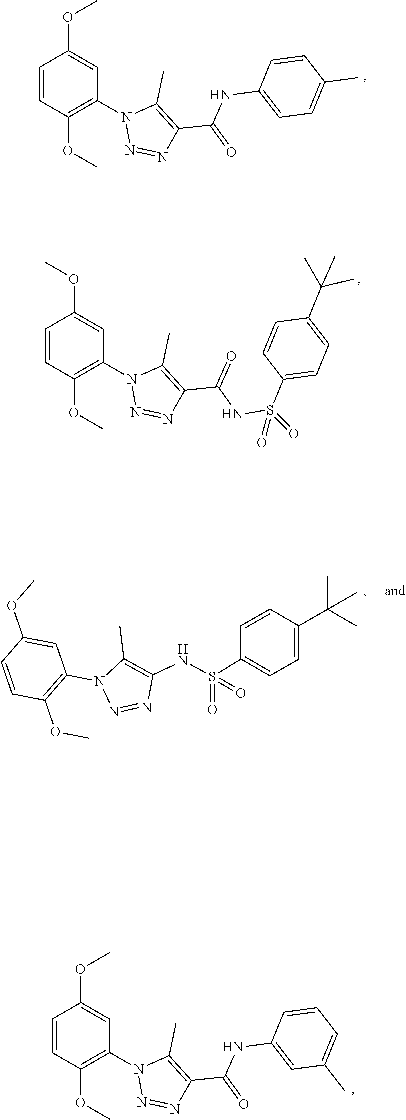 [SMILES: COc1ccc(OC)c(-n2nnc(C(=O)Nc3ccc(C)cc3)c2C)c1 ; COc1ccc(OC)c(-n2nnc(C(=O)NS(=O)(=O)c3ccc(C(C)(C)C)cc3)c2C)c1 ; COc1ccc(OC)c(-n2nnc(NS(=O)(=O)c3ccc(C(C)(C)C)cc3)c2C)c1 ; COc1ccc(OC)c(-n2nnc(C(=O)Nc3cccc(C)c3)c2C)c1]

or a pharmaceutically acceptable salt thereof.

In various aspects, Q² is selected from N and CH.

In various aspects, R⁵ is C1-C4 alkoxy.

In various aspects, R⁶ is C1-C4 alkoxy.

In various aspects, R⁸ is hydrogen. In a further aspect, R¹ is C1-C4 alkoxy.

In various aspects, the compound has a structure represented by a formula:

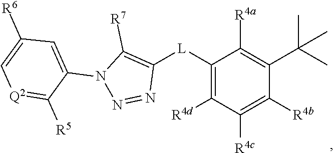

or a pharmaceutically acceptable salt thereof.

In various aspects, the compound has a structure represented by a formula:

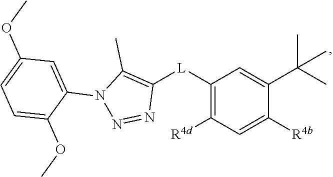

or a pharmaceutically acceptable salt thereof.

In various aspects, the compound is not:

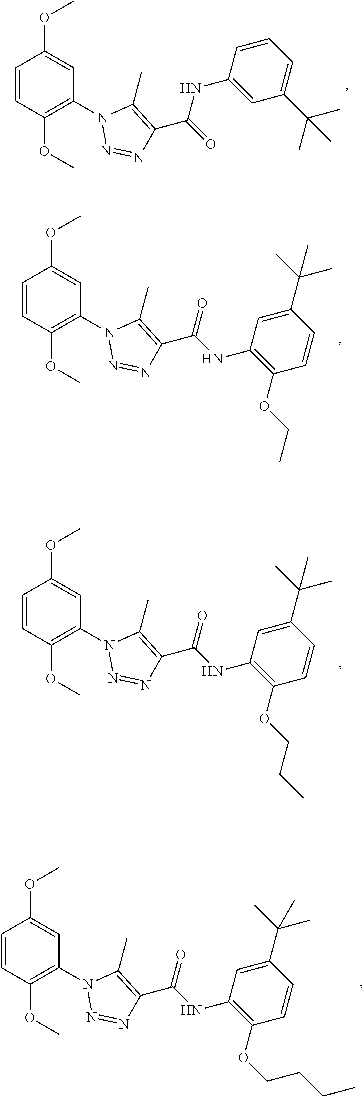 [SMILES: COc1ccc(OC)c(-n2nnc(C(=O)Nc3cccc(C(C)(C)C)c3)c2C)c1 ; CCOc1ccc(C(C)(C)C)cc1NC(=O)c1nnn(-c2cc(OC)ccc2OC)c1C ; CCCOc1ccc(C(C)(C)C)cc1NC(=O)c1nnn(-c2cc(OC)ccc2OC)c1C ; CCCCOc1ccc(C(C)(C)C)cc1NC(=O)c1nnn(-c2cc(OC)ccc2OC)c1C]

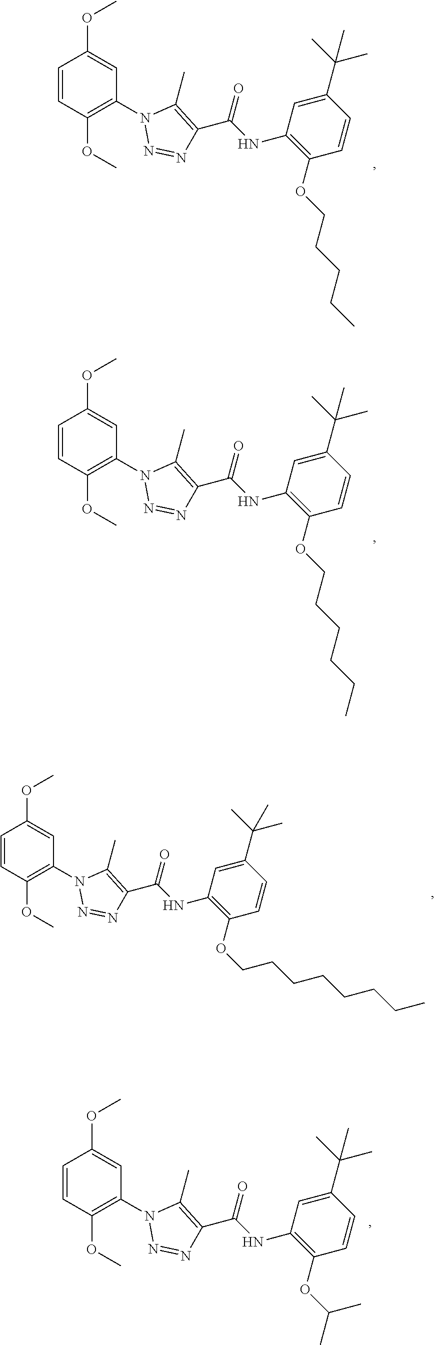 [SMILES: CCCCCOc1ccc(C(C)(C)C)cc1NC(=O)c1nnn(-c2cc(OC)ccc2OC)c1C ; CCCCCCOc1ccc(C(C)(C)C)cc1NC(=O)c1nnn(-c2cc(OC)ccc2OC)c1C ; CCCCCCCCOc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OC(C)C)c2C)c1]

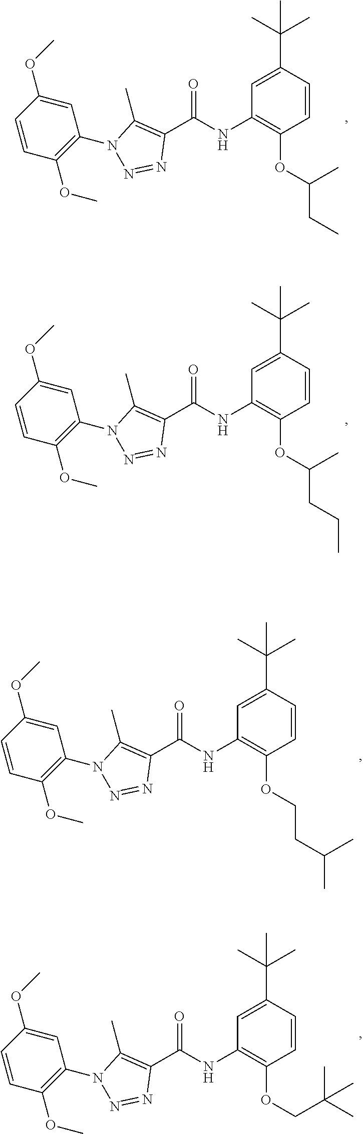 [SMILES: CCC(C)Oc1ccc(C(C)(C)C)cc1NC(=O)c1nnn(-c2cc(OC)ccc2OC)c1C ; CCCC(C)Oc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OCCC(C)C)c2C)c1 ; COc1ccc(OC)c(-n2nnc(C(=O)Nc3cc(C(C)(C)C)ccc3OCC(C)(C)C)c2C)c1]

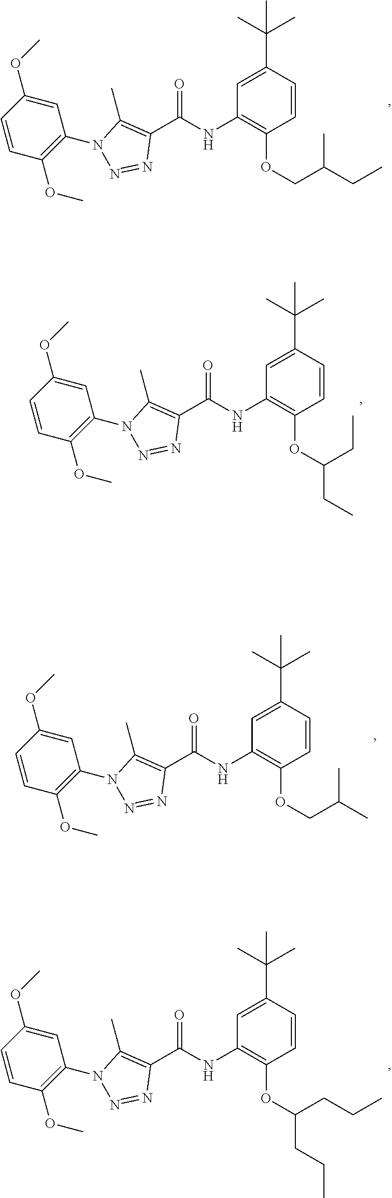 [SMILES: CCC(C)COc1ccc(C(C)(C)C)cc1NC(=O)c1nnn(-c2cc(OC)ccc2OC)c1C ; CCC(CC)Oc1ccc(C(C)(C)C)cc1NC(=O)c1nnn(-c2cc(OC)ccc2OC)c1C ; COc1ccc(OC)c(-n2nnc(C(=O)Nc3cc(C(C)(C)C)ccc3OCC(C)C)c2C)c1 ; CCCC(CCC)Oc1ccc(C(C)(C)C)cc1NC(=O)c1nnn(-c2cc(OC)ccc2OC)c1C]

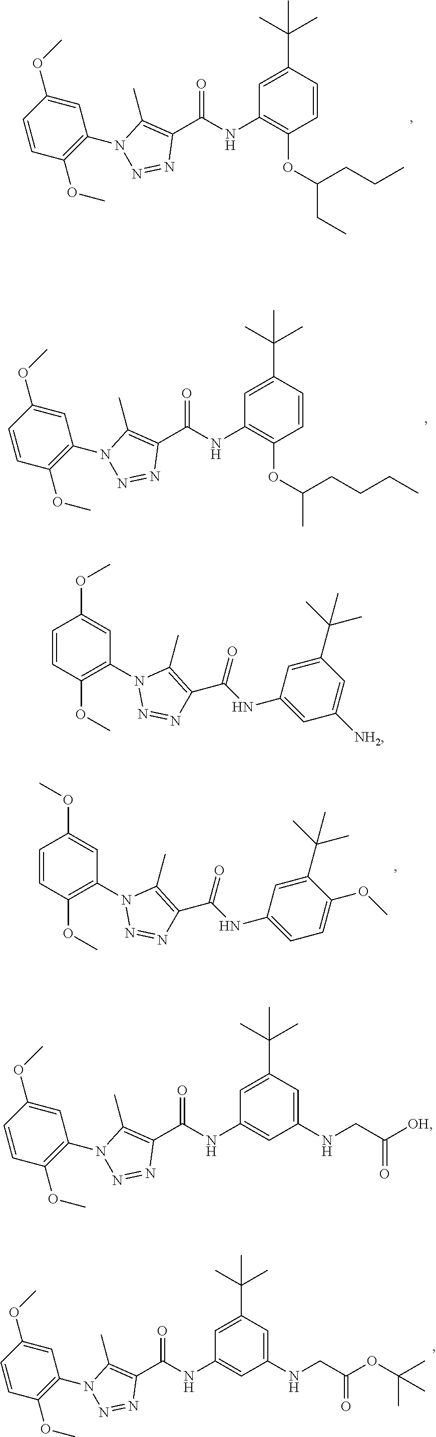 [SMILES: CCCC(CC)Oc1ccc(C(C)(C)C)cc1NC(=O)c1nnn(-c2cc(OC)ccc2OC)c1C ; CCCCC(C)Oc1ccc(C(C)(C)C)cc1NC(=O)c1nnn(-c2cc(OC)ccc2OC)c1C ; COc1ccc(OC)c(-n2nnc(C(=O)Nc3cc(N)cc(C(C)(C)C)c3)c2C)c1 ; COc1ccc(OC)c(-n2nnc(C(=O)Nc3ccc(OC)c(C(C)(C)C)c3)c2C)c1 ; COc1ccc(OC)c(-n2nnc(C(=O)Nc3cc(NCC(=O)O)cc(C(C)(C)C)c3)c2C)c1 ; COc1ccc(OC)c(-n2nnc(C(=O)Nc3cc(NCC(=O)OC(C)(C)C)cc(C(C)(C)C)c3)c2C)c1]

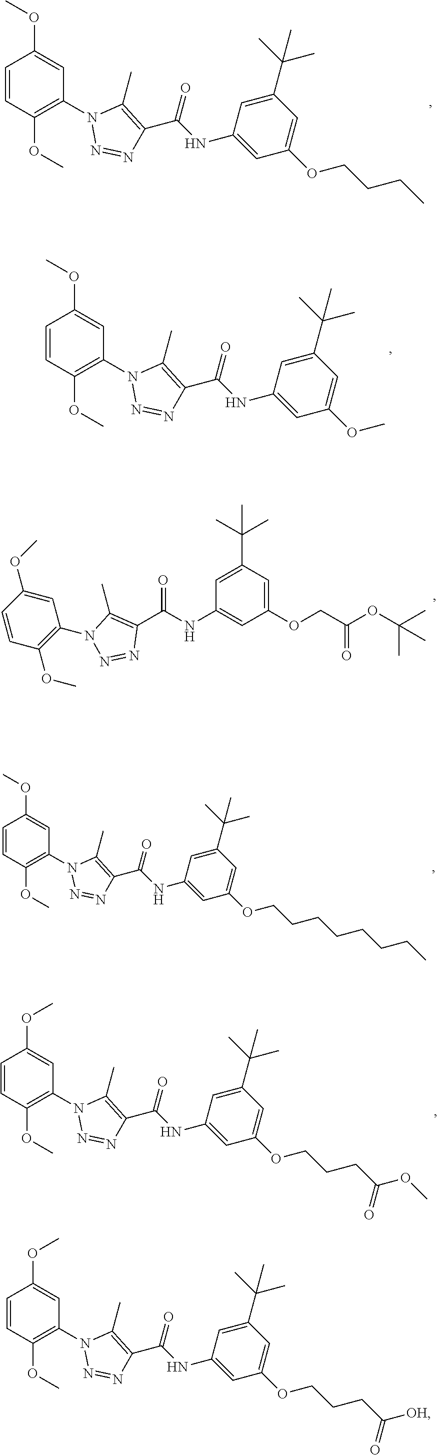 [SMILES: CCCCOc1cc(NC(=O)c2nnn(-c3cc(OC)ccc3OC)c2C)cc(C(C)(C)C)c1 ; COc1cc(NC(=O)c2nnn(-c3cc(OC)ccc3OC)c2C)cc(C(C)(C)C)c1 ; COc1ccc(OC)c(-n2nnc(C(=O)Nc3cc(OCC(=O)OC(C)(C)C)cc(C(C)(C)C)c3)c2C)c1 ; CCCCCCCCOc1cc(NC(=O)c2nnn(-c3cc(OC)ccc3OC)c2C)cc(C(C)(C)C)c1 ; COC(=O)CCCOc1cc(NC(=O)c2nnn(-c3cc(OC)ccc3OC)c2C)cc(C(C)(C)C)c1 ; COc1ccc(OC)c(-n2nnc(C(=O)Nc3cc(OCCCC(=O)O)cc(C(C)(C)C)c3)c2C)c1]

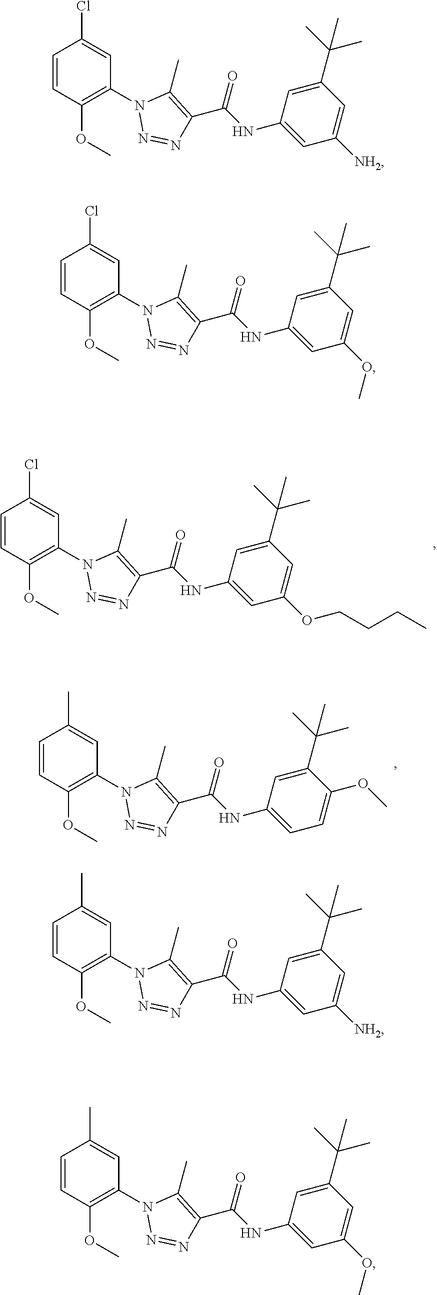

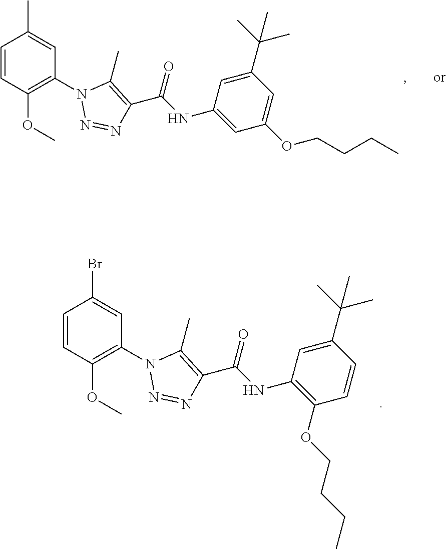 [SMILES: CCCCOc1cc(NC(=O)c2nnn(-c3cc(C)ccc3OC)c2C)cc(C(C)(C)C)c1 ; CCCCOc1ccc(C(C)(C)C)cc1NC(=O)c1nnn(-c2cc(Br)ccc2OC)c1C]

In various aspects, the compound is selected from:

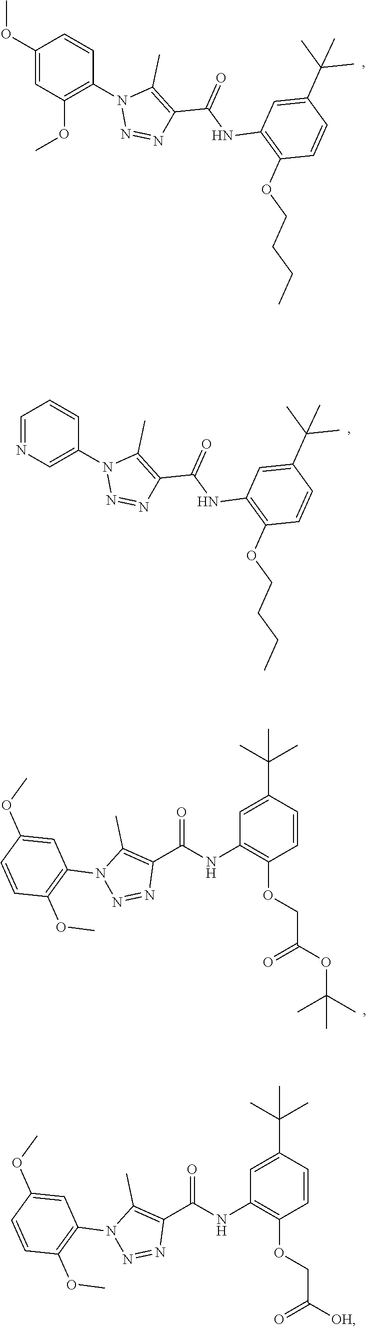 [SMILES: CCCCOc1ccc(C(C)(C)C)cc1NC(=O)c1nnn(-c2ccc(OC)cc2OC)c1C ; CCCCOc1ccc(C(C)(C)C)cc1NC(=O)c1nnn(-c2cccnc2)c1C ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1]

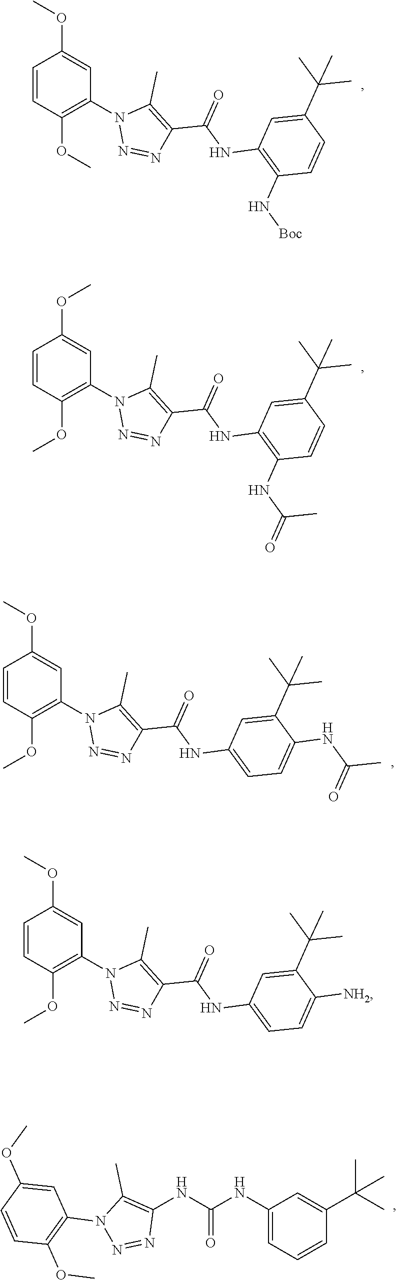 [SMILES: COc1ccc(OC)c(-n2nnc(C(=O)Nc3cc(C(C)(C)C)ccc3NC(=O)OC(C)(C)C)c2C)c1 ; COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1 ; COc1ccc(OC)c(-n2nnc(NC(=O)Nc3cccc(C(C)(C)C)c3)c2C)c1]

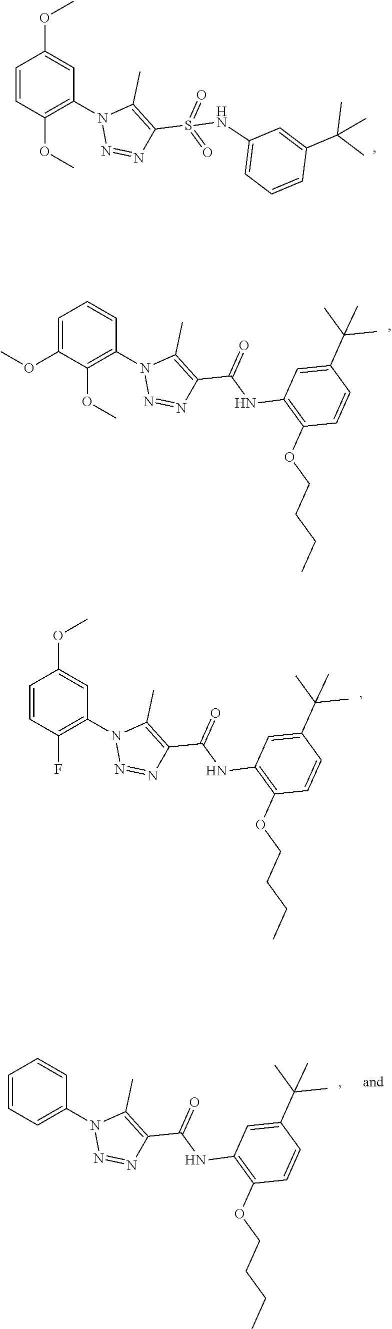

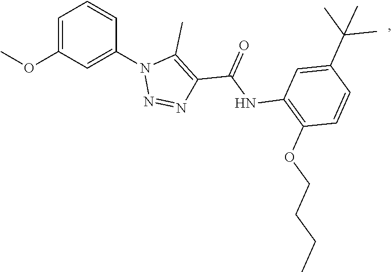 [SMILES: CCCCOc1ccc(C(C)(C)C)cc1NC(=O)c1nnn(-c2cccc(OC)c2)c1C]

or a pharmaceutically acceptable salt thereof.

In a further aspect, the compound is selected from:

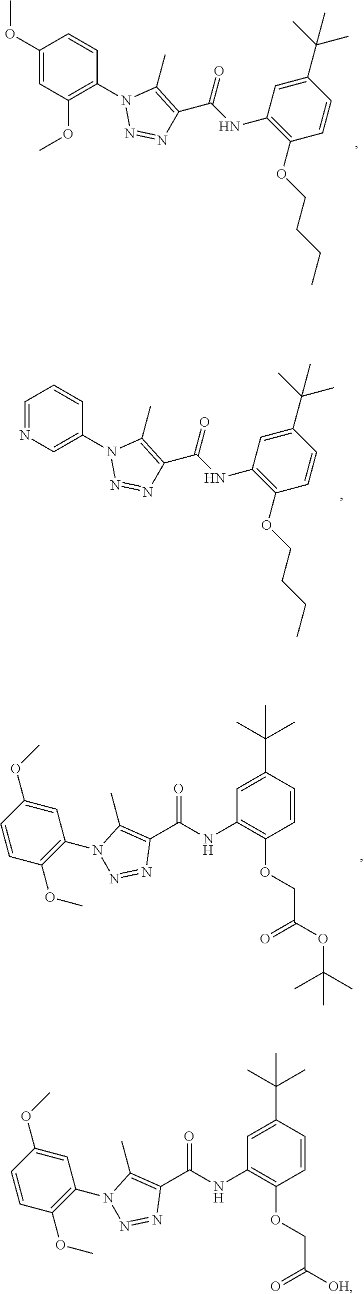 [SMILES: CCCCOc1ccc(C(C)(C)C)cc1NC(=O)c1nnn(-c2ccc(OC)cc2OC)c1C ; CCCCOc1ccc(C(C)(C)C)cc1NC(=O)c1nnn(-c2cccnc2)c1C ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1]

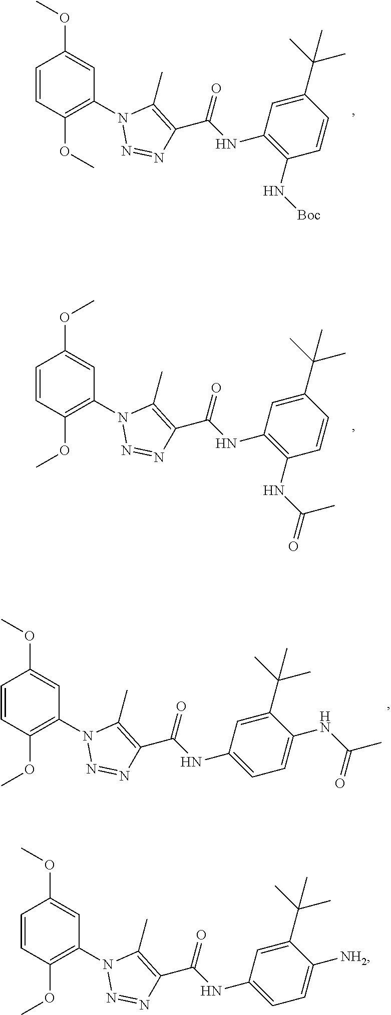 [SMILES: COc1ccc(OC)c(-n2nnc(C(=O)Nc3cc(C(C)(C)C)ccc3NC(=O)OC(C)(C)C)c2C)c1 ; COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1]

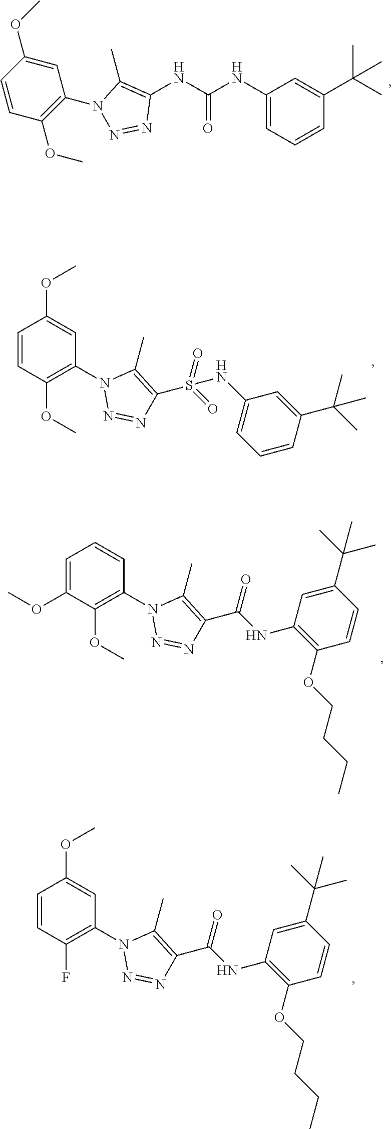

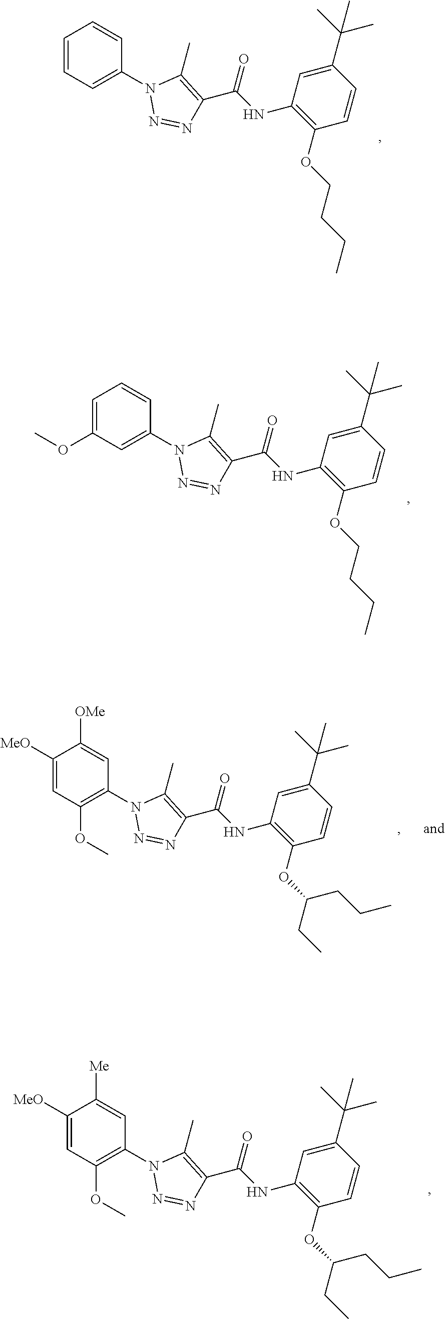

or a pharmaceutically acceptable salt thereof.

In a further aspect, the subject is a mammal. In a still further aspect, the mammal is human.

In a further aspect, the subject has been diagnosed with a need for treatment of the disorder prior to the administering step. In a still further aspect, the subject is at risk for developing the disorder prior to the administering step.

In a further aspect, the method further comprises identifying a subject at risk for developing the disorder prior to the administering step.

In a further aspect, the bowel disorder is selected from irritable bowel syndrome (IBS), Crohn's disease, celiac disease, and intestinal obstruction. In a still further aspect, the bowel disorder is irritable bowel syndrome (IBS).

### J. Additional Methods of Using the Compositions

Provided are methods of using of a disclosed composition or medicament. In one aspect, the method of use is directed to the treatment of a disorder. In a further aspect, the disclosed compounds can be used as single agents or in combination with one or more other drugs in the treatment, prevention, control, amelioration, or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

### 1. Manufacture of a Medicament

In one aspect, the invention relates to a method for the manufacture of a medicament for treating a disorder associated with dysfunctional PXR activity such as, for example, a disorder of uncontrolled cellular proliferation and a bowel disorder, in a mammal, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in modulating PXR activity as further disclosed herein. The dose administered to an animal, particularly a human, in the context of the present invention should be sufficient to affect a therapeutic response in the animal over a reasonable time-frame. One skilled in the art will recognize that dosage will depend upon a variety of factors including the condition of the animal, the body weight of the animal, as well as the severity and stage of the disorder.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 2. Use of Compounds and Compositions

Also provided are the uses of the disclosed compounds and compositions. Thus, in one aspect, the invention relates to the use of the disclosed compounds and compositions as modulators of PXR activity.

In a further aspect, the invention relates to the use of a disclosed compound or product of a disclosed method in the manufacture of a medicament for the treatment of a disorder associated with uncontrolled cellular proliferation, for example, cancer, including, but not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and in another example acute lymphoblastic leukemia.

In a further aspect, the invention relates to the use of the disclosed compounds and compositions in decreasing an adverse drug reaction.

In a further aspect, the invention relates to the use of the disclosed compounds and compositions in treating a bowel disorder such as, for example, irritable bowel syndrome.

In a further aspect, the use relates to a process for preparing a pharmaceutical composition comprising a therapeutically effective amount of a disclosed compound or a product of a disclosed method, and a pharmaceutically acceptable carrier, for use as a medicament.

In a further aspect, the use relates to a process for preparing a pharmaceutical composition comprising a therapeutically effective amount of a disclosed compound or a product of a disclosed method, wherein a pharmaceutically acceptable carrier is intimately mixed with a therapeutically effective amount of the disclosed compound or the product of a disclosed method.

In various aspects, the use relates to the treatment of uncontrolled cellular proliferation in a vertebrate animal. In a further aspect, the use relates to the treatment of uncontrolled cellular proliferation in a human subject.

In a further aspect, the use is the treatment of uncontrolled cellular proliferation, for example cancer, for example a cancer selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and, in another example, acute lymphoblastic leukemia.

In various aspects, the use relates to decreasing an adverse drug reaction in a vertebrate animal. In a further aspect, the use relates to decreasing an adverse drug reaction in a human subject.

In a further aspect, the use is decreasing an adverse drug reaction such as, for example an adverse drug reaction associated with administration of an anticancer agent, an antibacterial agent, a non-steroidal anti-inflammatory agent, or an anticonvulsant.

In various aspects, the use relates to the treatment of a bowel disorder in a vertebrate animal. In a further aspect, the use relates to the treatment of a bowel disorder in a human subject.

In a further aspect, the use is the treatment of a bowel disorder such as, for example, irritable bowel syndrome (IBS), Crohn's disease, celiac disease, and intestinal obstruction.

It is understood that the disclosed uses can be employed in connection with the disclosed compounds, methods, compositions, and kits. In a further aspect, the invention relates to the use of a disclosed compound or composition of a medicament for the treatment of a disorder or condition associated with dysfunctional PXR activity in a mammal.

In a further aspect, the invention relates to the use of a disclosed compound or composition in the manufacture of a medicament for the treatment of a disorder or condition associated with dysfunctional PXR activity such as, for example, cancer, a bowel disorder, and an adverse drug reaction.

### 9. Kits

In various aspects, the agents and pharmaceutical compositions described herein can be provided in a kit. The kit can also include combinations of the agents and pharmaceutical compositions described herein.

Thus, in one aspect, disclosed are kits comprising a disclosed compound and one or more selected from: (a) an agent known to increase pregnane X receptor (PXR) activity; (b) an agent known for the treatment of a disorder associated with pregnane X receptor (PXR) dysfunction; (c) administering the compound in connection with a disorder associated with pregnane X receptor (PXR) dysfunction; (d) instructions for decreasing an adverse drug reaction; and (e) instructions for treating a disorder associated with pregnane X receptor (PXR) dysfunction.

In a further aspect, the compound has a structure represented by a formula:

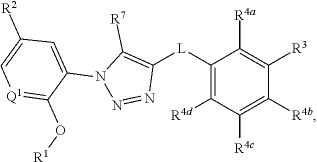

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

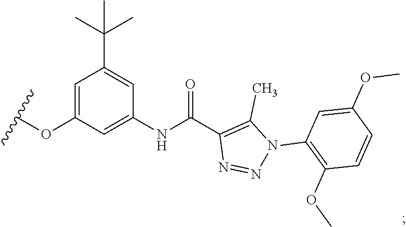

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl, and wherein R¹⁰ is selected from hydrogen and C1-C4 alkyl, or a pharmaceutically acceptable salt thereof.

In a further aspect, the compound has a structure represented by a formula:

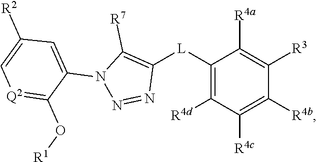

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is C1-C8 alkyl; wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula:

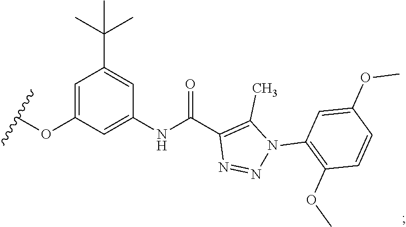

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and wherein R¹⁰ is selected from hydrogen and C1-C4 alkyl, or a pharmaceutically acceptable salt thereof.

In a further aspect, the compound is selected from:

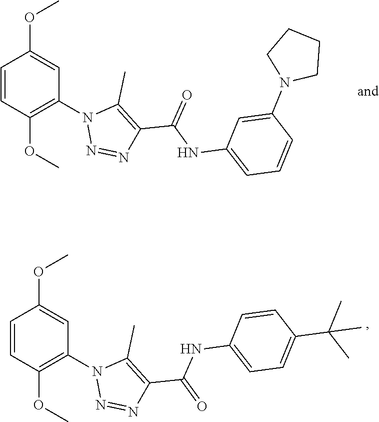 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1]

or a pharmaceutically acceptable salt thereof.

In one aspect, disclosed are kits comprising a compound having a structure represented by a formula:

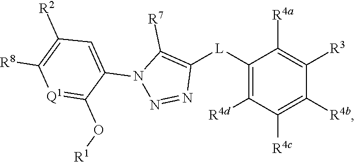

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q¹ is selected from N and CH; wherein R¹ is C1-C4 alkyl; wherein R² is selected from C1-C4 haloalkyl, C1-C4 alkoxy, and C1-C4 haloalkoxy; wherein R³ is selected from C1-C8 alkyl, —CO₂(C1-C4 alkyl), and —C(O)Cy²; wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy³; wherein Cy³, when present, is a C2-C5 heterocycloalkyl substituted with 0 or 1 group selected from C1-C4 alkyl and —C(O)(C1-C4 alkyl); wherein each of R^{4a}, R^{4b}, R⁴¹, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)ₙOR¹⁴, —NHR¹⁵, —B(OR¹⁶)₂, —OCy¹, and Cy¹; wherein n, when present, is selected from 1, 2, 3, 4, and 5; wherein R¹³, when present, is selected from halogen, —CN, —NH₂, —OH, —C≡CH, —CHO, —CO₂H, CO₂(C1-C4 alkyl), C1-C4 alkylamino, (C1-C4)(C1-C4) dialkylamino, unsubstituted morpholine, and a structure represented by a formula:

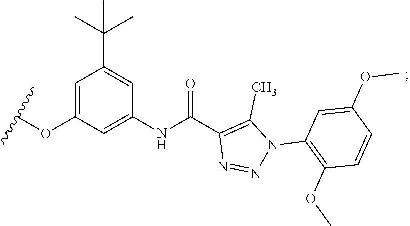

wherein R¹⁴, when present, is selected from hydrogen and C1-C4 alkyl; wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl); wherein each occurrence of R¹⁶, when present, is independently selected from hydrogen and C1-C4 alkyl, or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups; wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); wherein R⁷ is selected from hydrogen and C1-C4 alkyl; wherein R⁸ is selected from hydrogen and halogen; and wherein R¹⁰ is selected from hydrogen and C1-C4 alkyl, or a pharmaceutically acceptable salt thereof, and one or more selected from: (a) an agent known to increase pregnane X receptor (PXR) activity; (b) an agent known for the treatment of a disorder associated with pregnane X receptor (PXR) dysfunction; (c) administering the compound in connection with a disorder associated with pregnane X receptor (PXR) dysfunction; (d) instructions for decreasing an adverse drug reaction; and (e) instructions for treating a disorder associated with pregnane X receptor (PXR) dysfunction.

In one aspect, disclosed are kits comprising a compound selected from:

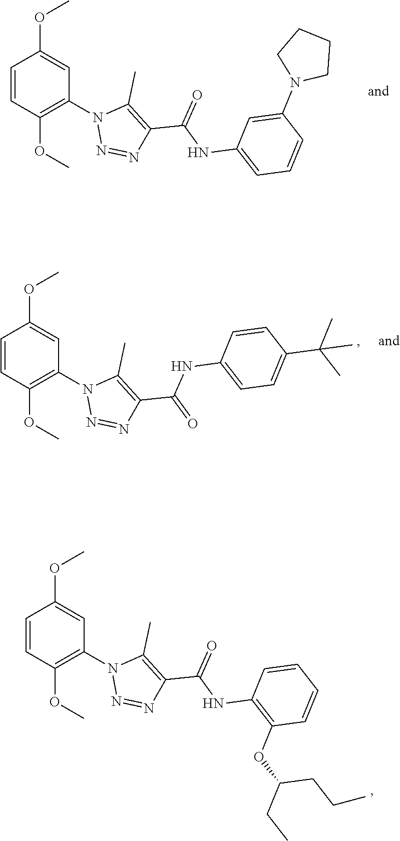 [SMILES: COc1ccc(OC)c(-n2nnc(C(=O)Nc3cccc(N4CCCC4)c3)c2C)c1 ; COc1ccc(OC)c(-n2nnc(C(=O)Nc3ccc(C(C)(C)C)cc3)c2C)c1 ; CCC[C@H](CC)Oc1ccccc1NC(=O)c1nnn(-c2cc(OC)ccc2OC)c1C]

or a pharmaceutically acceptable salt thereof, and one or more selected from: (a) an agent known to increase pregnane X receptor (PXR) activity; (b) an agent known for the treatment of a disorder associated with pregnane X receptor (PXR) dysfunction; (c) administering the compound in connection with a disorder associated with pregnane X receptor (PXR) dysfunction; (d) instructions for decreasing an adverse drug reaction; and (e) instructions for treating a disorder associated with pregnane X receptor (PXR) dysfunction.

In one aspect, disclosed are kits comprising a compound having a structure represented by a formula:

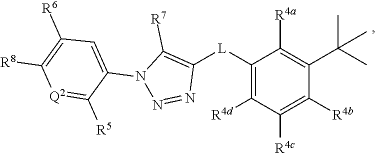

wherein L is selected from —NR¹⁰C(O)—, —N(R¹⁰)C(O)NR¹¹—, —C(O)NR¹⁰—, —SO₂NR¹⁰—, and —NR¹⁰SO₂—; wherein R¹⁰ is selected from hydrogen and C1-C4 alkyl; wherein R¹¹, when present, is selected from hydrogen and C1-C4 alkyl; wherein Q² is selected from N and CR¹; wherein R¹², when present, is selected from hydrogen and C1-C4 alkoxy; wherein each of R^{4a}, R^{4b}, R^{4c}, and R^{4d} is independently selected from hydrogen, —NH₂, C1-C8 alkoxy, —O—(C1-C8 alkyl)-R¹, and —NHR¹⁵; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; wherein R⁶ is selected from hydrogen and C1-C4 alkoxy; and wherein R⁷ is selected from hydrogen and C1-C4 alkyl; and wherein R⁸ is selected from hydrogen and C1-C4 alkoxy, or a pharmaceutically acceptable salt thereof, and one or more selected from: (a) an agent known for the treatment of a disorder associated with pregnane X receptor (PXR) dysfunction; (b) instructions for administering the compound in connection with a disorder associated with pregnane X receptor (PXR) dysfunction; (c) instructions for decreasing an adverse drug reaction; and (d) instructions for treating a disorder associated with pregnane X receptor (PXR) dysfunction.

In a further aspect, the compound and the agent known to increase PXR activity are co-formulated. In a still further aspect, the compound and the agent known to increase PXR activity are co-packaged.

In a further aspect, the agent known to increase PXR activity is selected from an agent known to treat a disorder of uncontrolled cellular proliferation (e.g., paclitaxel, irinotecan, leucovorin, dasatinib, erlotinib), an agent known to treat an infectious disease (e.g., an antibacterial agent such as, for example, isoniazid, rifampicin, and flucloxacillin), a non-steroidal anti-inflammatory agent (e.g., acetaminophen), and an anticonvulsant agent (e.g., phenytoin).

In a further aspect, the kit further comprises a plurality of dosage forms, the plurality comprising one or more doses; wherein each dose comprises an effective amount of the compound and the agent known to increase PXR activity. In a still further aspect, the effective amount is a therapeutically effective amount. In yet a further aspect, the effective amount is a prophylactically effective amount. In an even further aspect, each dose of the compound and the agent known to increase PXR activity are co-packaged. In a still further aspect, each dose of the compound and the agent known to increase PXR activity are co-formulated.

In a further aspect, the compound and the agent known for the treatment of a disorder associated with pregnane X receptor (PXR) dysfunction are co-formulated. In a still further aspect, the compound and the agent known for the treatment of a disorder associated with pregnane X receptor (PXR) dysfunction are co-packaged.

In a further aspect, the agent known for the treatment of a disorder associated with pregnane X receptor (PXR) dysfunction is an agent known to treat a disorder of uncontrolled cellular proliferation.

In a further aspect, the kit further comprises a plurality of dosage forms, the plurality comprising one or more doses; wherein each dose comprises an effective amount of the compound and the agent known for the treatment of a disorder associated with pregnane X receptor (PXR) dysfunction. In a still further aspect, the effective amount is a therapeutically effective amount. In yet a further aspect, the effective amount is a prophylactically effective amount. In an even further aspect, each dose of the compound and the agent known for the treatment of a disorder associated with pregnane X receptor (PXR) dysfunction. In a still further aspect, each dose of the compound and the agent known for the treatment of a disorder associated with pregnane X receptor (PXR) dysfunction are co-formulated.

In one aspect, disclosed are kits comprising a disclosed compound, and one or more selected from: (a) an agent known for the treatment of a disorder associated with pregnane X receptor (PXR) dysfunction; (b) instructions for administering the compound in connection with a disorder associated with pregnane X receptor (PXR) dysfunction; (c) instructions for decreasing an adverse drug reaction; and (d) instructions for treating a disorder associated with pregnane X receptor (PXR) dysfunction.

In a further aspect, the compound has a structure represented by a formula:

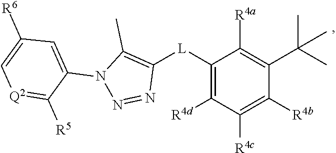

wherein Q² is selected from N and CR¹²; wherein R¹², when present, is selected from hydrogen and C1-C4 alkoxy; wherein each of R^{4a}, R^{4b}, R^{4c}, and R⁴⁶ is independently selected from hydrogen, —NH₂, C1-C8 alkoxy, —O—(C1-C8 alkyl)-R¹³, and —NHR¹¹; wherein R¹³, when present, is selected from —CO₂H and —CO₂(C1-C4 alkyl); wherein R¹⁵, when present, is selected from —C(O)(C1-C4 alkyl) and —CO₂(C1-C4 alkyl); wherein R⁵ is selected from hydrogen, halogen, and C1-C4 alkoxy; and wherein R⁶ is selected from hydrogen and C1-C4 alkoxy, or a pharmaceutically acceptable salt thereof.

In a further aspect, the compound is selected from:

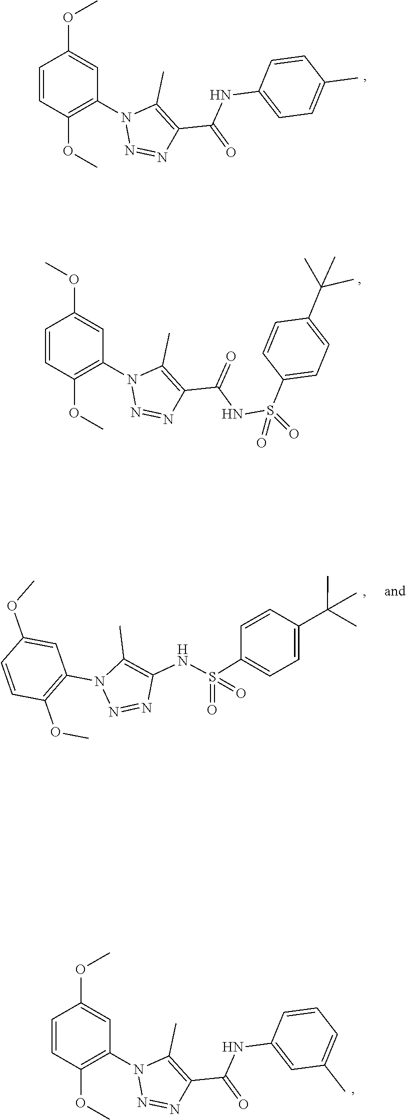 [SMILES: COc1ccc(OC)c(-n2nnc(C(=O)Nc3ccc(C)cc3)c2C)c1 ; COc1ccc(OC)c(-n2nnc(C(=O)NS(=O)(=O)c3ccc(C(C)(C)C)cc3)c2C)c1 ; COc1ccc(OC)c(-n2nnc(NS(=O)(=O)c3ccc(C(C)(C)C)cc3)c2C)c1 ; COc1ccc(OC)c(-n2nnc(C(=O)Nc3cccc(C)c3)c2C)c1]

or a pharmaceutically acceptable salt thereof.

In various aspects, the compound and the agent are co-formulated. In a further aspect, the compound and the agent are co-packaged.

In a further aspect, the disorder is a bowel disorder.

In a further aspect, the agent is selected from an anti-inflammatory agent, an immune system suppressor, a biologic, and antibiotic. In a still further aspect, the anti-inflammatory agent is selected from a corticosteroid and an aminosalicylate. In yet a further aspect, the anti-inflammatory agent is selected from mesalamine, balsalazie, and olsalazine. In an even further aspect, the immune system suppressor is selected from azathioprine, mercaptopurine, and methotrexate. In a still further aspect, the biologic is selected from infliximab, adalimumab, golimumab, certolizumab, vedolizumab, and ustekinumab. In yet a further aspect, the antibiotic is selected from ciprofloxacin and metronidazole.

In various aspects, the informational material can be descriptive, instructional, marketing or other material that relates to the methods described herein and/or to the use of the agents for the methods described herein. For example, the informational material may relate to the use of the agents herein to treat a subject who has, or who is at risk for developing, a disorder or condition associated with dysfunctional PXR activity such as, for example, cancer, a bowel disorder, and an adverse drug reaction. The kits can also include paraphernalia for administering the agents of this invention to a cell (in culture or in vivo) and/or for administering a cell to a patient.

In various aspects, the informational material can include instructions for administering the pharmaceutical composition and/or cell(s) in a suitable manner to treat a human, e.g., in a suitable dose, dosage form, or mode of administration (e.g., a dose, dosage form, or mode of administration described herein). In a further aspect, the informational material can include instructions to administer the pharmaceutical composition to a suitable subject, e.g., a human having, or at risk for developing, a disorder or condition associated with dysfunctional PXR activity such as, for example, cancer, a bowel disorder, and an adverse drug reaction.

In various aspects, the composition of the kit can include other ingredients, such as a solvent or buffer, a stabilizer, a preservative, a fragrance or other cosmetic ingredient. In such aspects, the kit can include instructions for admixing the agent and the other ingredients, or for using one or more compounds together with the other ingredients.

### 10. Subjects

In various aspects, the subject of the herein disclosed methods is a vertebrate, e.g., a mammal. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with a disorder or condition associated with dysfunctional PXR activity such as, for example, cancer, a bowel disorder, or an adverse drug reaction, prior to the administering step. In some aspects of the disclosed methods, the subject has been identified with a need for treatment prior to the administering step. In one aspect, a subject can be treated prophylactically with a disclosed compound or composition, as discussed elsewhere herein.

a. Dosage

Toxicity and therapeutic efficacy of the agents and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on a human's ability to hear. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

b. Routes of Administration

Also provided are routes of administering the disclosed compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In various aspects, the modes of administration described above may be combined in any order.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### K. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### 1. Chemistry Methods

a. General Methods and Synthesis

Organic reagents were purchased from commercial suppliers unless otherwise noted and were used without further purification. All solvents were analytical or reagent grade and the solvents were dried using the Glass Contour Solvent Systems by SG Water USA. All reactions with water- and/or air-sensitive starting materials were carried out in pre-dried glassware under argon atmosphere with standard procedure. Flash column chromatography was performed by using Biotage Isolera Flash Systems and Biotage SNAP Ultra or Biotage SNAP Ultra C18 columns. All reactions as well as compound purities were monitored by UPLC-MS by using a Waters Acquity UPLC MS system with a C18 column in a 2-min gradient [H₂O+0.1% formic acid (FA)→acetonitrile (ACN)+0.1% FA] and detectors of PDA (215-400 nm), ELSD, and Acquity SQD ESI-positive MS (Waters Corporation, Milford, MA). High-resolution mass spectra were determined by using a Waters Acquity UPLC system with a C18 column (H₂O+0.1% FA→ACN+0.10% FA gradient over 2.5 min) and Xevo G2Q-TOF ESI-positive MS in resolution mode. Compounds were internally normalized to leucine-enkephalin lock solution, with a calculated error of <3 ppm. All final compounds used for SAR studies have purity at 95% or greater by HPLC. All NMR spectra were recorded on Bruker 500 MHz spectrometer in the solvents indicated, and spectra were processed using MestReNova (14.1.0). The chemical shift values are expressed in parts per million (ppm) relative to tetramethylsilane as the internal standard. Coupling constants (J) are reported in hertz (Hz).

b. Preparation of A1

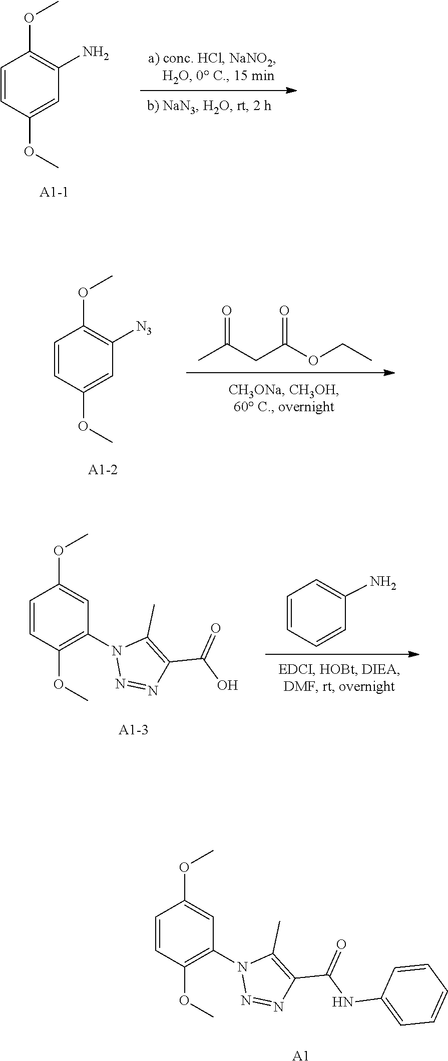

To a solution of 2,5-dimethoxyaniline (7.66 g, 50 mmol) in water (50 ml) was added hydrochloric acid (37%, 30 ml) with stirring at 0° C. Then a solution of sodium nitrite (3.45 g, 50.0 mmol) in water (10 ml) was added dropwise to the mixture at 0° C. After being stirred for 15 min, hexane (50 mL) was added to the reaction mixture. Then a solution of NaN₃ (3.58 g, 55.0 mmol) in water (10 ml) was added dropwise at 0° C. The reaction mixture was stirred at 0° C. for 15 min and then kept at ambient temperature for 1.5 hrs. The mixture was extracted with hexane 150 mL, and then the organic layer was washed with water (2×50 mL) and once with brine (50 mL). The organic layer was dried over MgSO₄, filtered, and concentrated under reduced pressure to provide an oil. The residue was used directly for the next step without further purification.

To the residue was added CH₃OH 100 mL, ethyl acetoacetate (7.65 ml, 60 mmol) and CH₃ONa (10.8 g, 200 mmol). The reaction mixture was stirred at 60° C. for overnight. The mixture was evaporated to remove solvent. The residue was added NaOH (8 g, 200 mmol) and water 100 mL, and stirred at ambient temperature for 3 hrs. Then the pH was adjusted to around 4 with hydrochloric acid (37%). The mixture was extracted with ethyl acetate (2×200 mL) and dried over MgSO₄, filtered, and concentrated under reduced pressure to provide 1-(2,5-dimethoxyphenyl)-5-methyl-1H-1,2,3-triazole-4-carboxylic acid (7.4 g, 56% yield) as a brown solid. ¹H NMR (400 MHz, DMSO-d6) δ 13.10 (s, 1H), 7.26 (d, J=9.2 Hz, 1H), 7.20 (dd, J=9.1, 3.1 Hz, 1H), 7.11 (d, J=3.0 Hz, 1H), 3.76 (s, 3H), 3.74 (s, 3H), 2.31 (s, 3H). ¹³C NMR (101 MHz, DMSO-d6) δ 162.56, 153.04, 147.62, 140.31, 135.76, 123.71, 117.32, 113.93, 113.82, 56.27, 55.81, 9.06.

To a solution of 1-(2,5-dimethoxyphenyl)-5-methyl-1H-1,2,3-triazole-4-carboxylic acid (0.527 g, 2 mmol) in DMF 5 mL was added aniline (0.219 ml, 2.400 mmol) and 3-(3-Dimethylaminopropyl)-1-ethyl-carbodiimide hydrochloride (0.4608 g, 3.00 mmol), followed by N-ethyl-N-isopropylpropan-2-amine (0.517 g, 4.00 mmol) at ambient temperature. The reaction mixture was stirred for overnight. The rection mixture was diluted with water (30 mL) and extracted with ethyl acetate (2×20 mL). The combined organic layers were washed with brine (50 mL) and dried over MgSO₄. After filtration, the extract was evaporated in vacuum. The residue was purified by silica gel chromatography (0% to 100% EtOAc in hexane) to get 1-(2,5-dimethoxyphenyl)-5-methyl-N-phenyl-1H-1,2,3-triazole-4-carboxamide (0.456 g, 67% yield) as a white solid. ¹H NMR (400 MHz, Chloroform-d) δ 9.08 (s, 1H), 7.75-7.68 (m, 2H), 7.41-7.35 (m, 2H), 7.17-7.07 (m, 2H), 7.03 (d, J=9.1 Hz, 1H), 6.95 (d, J=2.9 Hz, 1H), 3.82 (s, 3H), 3.76 (s, 3H), 2.52 (s, 3H). ¹³C NMR (101 MHz, Chloroform-d) δ 159.41, 153.71, 148.03, 139.54, 137.86, 137.80, 129.08, 124.33, 124.25, 119.81, 117.51, 113.82, 113.35, 56.34, 56.01, 9.22. ESI-TOF HRMS: m/z 339.1453 (C₁₈H₁₈N₄O₃+H⁺ requires 339.1452).

c. Preparation of A2

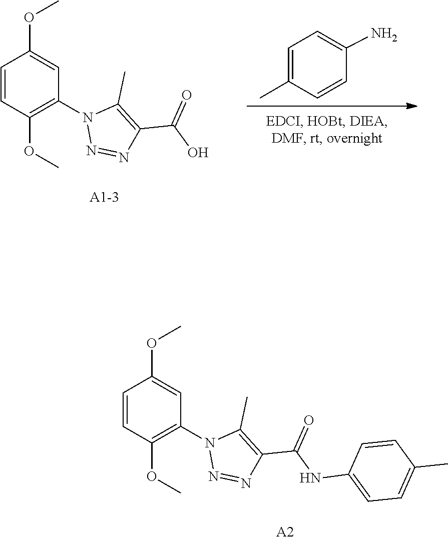

The preparation of compound A2 was prepared by reaction of 1-(2,5-dimethoxyphenyl)-5-methyl-1H-1,2,3-triazole-4-carboxylic acid and p-toluidine according to the procedure for A1 synthesis. light brown solid, 0.42 g, 60% yield. ¹H NMR (400 MHz, Chloroform-d) δ 9.02 (s, 1H), 7.60 (d, J=8.4 Hz, 2H), 7.20-7.14 (m, 2H), 7.08 (dd, J=9.1, 3.0 Hz, 1H), 7.02 (d, J=9.1 Hz, 1H), 6.94 (d, J=3.0 Hz, 1H), 3.81 (s, 3H), 3.75 (s, 3H), 2.51 (s, 3H), 2.34 (s, 3H). ¹³C NMR (101 MHz, CDCl₃) δ 159.32, 153.69, 148.03, 139.39, 137.95, 135.25, 133.83, 129.56, 124.36, 119.82, 117.46, 113.83, 113.34, 56.33, 56.00, 20.91, 9.21. ESI-TOF HRMS: m/z 353.1608 (C₁₉H₂₀N₄O₃+H⁺ requires 353.1608).

d. Preparation of A3

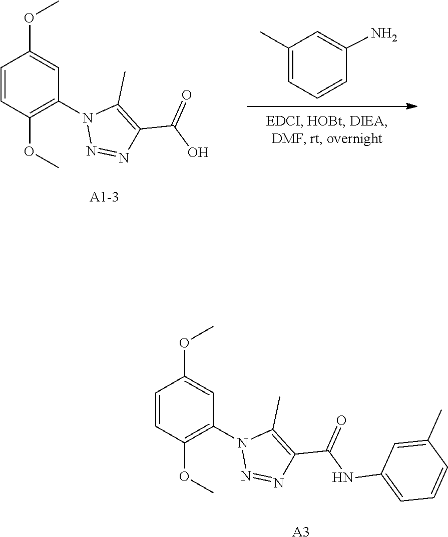

The preparation of compound A3 was prepared by reaction of 1-(2,5-dimethoxyphenyl)-5-methyl-1H-1,2,3-triazole-4-carboxylic acid and m-toluidine according to the procedure for A1 synthesis. Light brown solid, 0.562 g, 80% yield. ¹H NMR (400 MHz, CDCl₃) δ 9.05 (s, 1H), 7.61 (d, J=2.2 Hz, 1H). 7.47 (d, J=7.6 Hz, 1H), 7.30-7.21 (m, 1H), 7.09 (ddd, J=9.1, 3.0, 1.1 Hz, 1H), 7.03 (dd, J=9.2, 1.1 Hz, 1H), 6.98-6.93 (m, 2H), 3.82 (d, J=1.0 Hz, 3H), 3.76 (d, J=1.1 Hz, 3H), 2.52 (d, J=1.0 Hz, 3H), 2.38 (s, 3H). 13C NMR (101 MHz, CDCl₃) δ 159.35, 153.71, 148.03, 139.52, 139.01, 137.87, 137.72, 128.89, 125.07, 124.32, 120.44, 117.52, 116.87, 113.80, 113.34, 56.35, 56.01, 21.56, 9.23.

ESI-TOF HRMS: m/z 353.1602 (C₁₉H₂₀N₄O₃+H⁺ requires 353.1608).

e. Preparation of A4

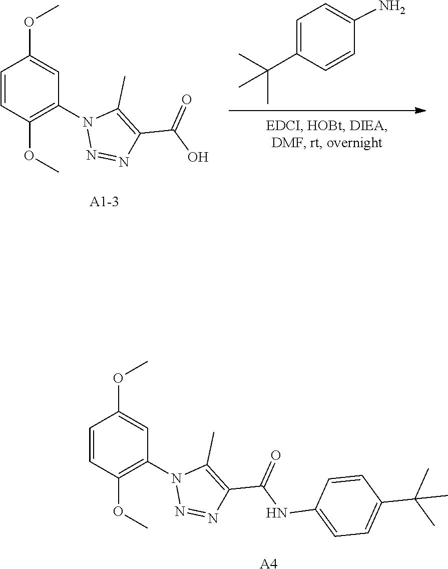 [SMILES: COc1ccc(OC)c(-n2nnc(C(=O)O)c2C)c1 ; CC(C)(C)c1ccc(N)cc1 ; COc1ccc(OC)c(-n2nnc(C(=O)Nc3ccc(C(C)(C)C)cc3)c2C)c1 ; "A4"]

The preparation of compound A4 was prepared by reaction of 1-(2,5-dimethoxyphenyl)-5-methyl-1H-1,2,3-triazole-4-carboxylic acid and 4-(tert-butyl)aniline according to the procedure for A1 synthesis. White solid, 625 mg, 79% yield. ¹H NMR (400 MHz, Chloroform-d) δ 9.04 (s, 1H), 7.69-7.56 (m, 2H), 7.43-7.36 (m, 2H), 7.09 (dd, J=9.2, 3.0 Hz, 1H), 7.03 (d, J=9.1 Hz, 1H), 6.95 (d, J=3.0 Hz, 1H), 3.82 (d, J=1.1 Hz, 3H), 3.77 (d, J=1.0 Hz, 3H), 2.52 (d, J=0.9 Hz, 3H), 1.33 (d, J=0.9 Hz, 9H). ¹³C NMR (101 MHz, CDCl₃) δ 159.31, 153.70, 148.04, 147.20, 139.41, 137.95, 135.16, 125.88, 124.36, 119.61, 117.49, 113.82, 113.34, 56.34, 56.00, 34.41, 31.40, 9.22. ESI-TOF HRMS: m/z 395.2078 (C₂₂H₂₆N₄O₃+H⁺ requires 395.2077).

f. Preparation of A5

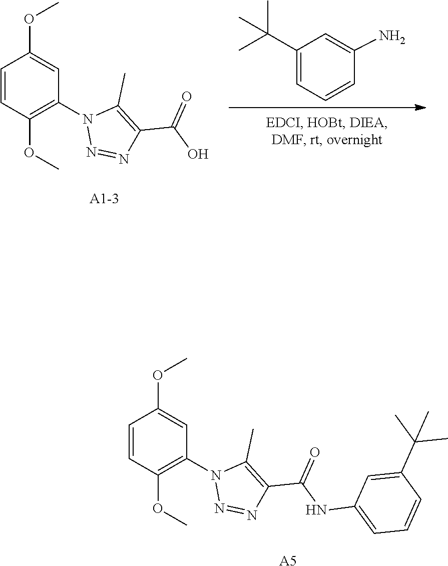 [SMILES: COc1ccc(OC)c(-n2nnc(C(=O)O)c2C)c1 ; CC(C)(C)c1cccc(N)c1 ; COc1ccc(OC)c(-n2nnc(C(=O)Nc3cccc(C(C)(C)C)c3)c2C)c1 ; "A5"]

The preparation of compound A5 was prepared by reaction of 1-(2,5-dimethoxyphenyl)-5-methyl-1H-1,2,3-triazole-4-carboxylic acid and 3-(tert-butyl)aniline according to the procedure for A1 synthesis. White solid, 653 mg, 83% yield. ¹H NMR (400 MHz, Chloroform-d) δ 9.08 (s, 1H), 7.69 (t, J=2.0 Hz, 1H), 7.61 (ddd, J=8.0, 2.2, 1.0 Hz, 1H), 7.31 (t, J=7.9 Hz, 1H), 7.18 (ddd, J=7.9, 1.9, 1.0 Hz, 1H), 7.09 (dd, J=9.1, 3.0 Hz, 1H), 7.03 (d, J=9.2 Hz, 1H), 6.96 (d, J=3.0 Hz, 1H), 3.82 (s, 3H), 3.76 (s, 3H), 2.53 (s, 3H), 1.35 (s, 9H). ¹³C NMR (101 MHz, CDCl₃) δ 159.37, 153.71, 152.30, 148.04, 139.41, 137.98, 137.57, 128.71, 124.36, 121.33, 117.49, 117.07, 117.00, 113.83, 113.34, 56.34, 56.01, 34.81, 31.34, 9.24. ESI-TOF HRMS: m/z 395.2079 (C₂₂H₂₆N₄O₃+H⁺ requires 395.2077).

g. Preparation of A6

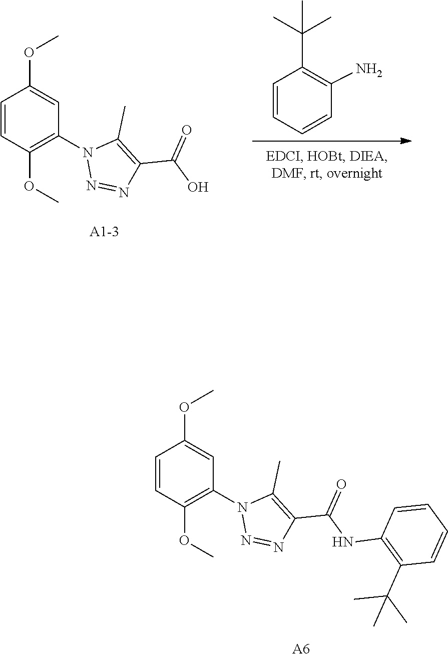 [SMILES: COc1ccc(OC)c(-n2nnc(C(=O)O)c2C)c1 ; CC(C)(C)c1ccccc1N ; COc1ccc(OC)c(-n2nnc(C(=O)Nc3ccccc3C(C)(C)C)c2C)c1 ; "A6"]

The preparation of compound A6 was prepared by reaction of 1-(2,5-dimethoxyphenyl)-5-methyl-1H-1,2,3-triazole-4-carboxylic acid and 2-(tert-butyl)aniline according to the procedure for A1 synthesis. White solid, 653 mg, 83% yield. ¹H NMR (400 MHz, DMSO-d₆) δ 9.71 (s, 1H), 7.51 (dd, J=7.5, 1.9 Hz, 1H), 7.45 (dd, J=7.6, 1.9 Hz, 1H), 7.32-7.16 (m, 5H), 3.78 (s, 3H), 3.77 (s, 3H), 2.39 (s, 3H), 1.43 (s, 9H). ¹³C NMR (101 MHz, DMSO) δ 159.52, 153.07, 147.66, 144.10, 138.72, 137.48, 135.27, 128.87, 126.55, 126.37, 126.21, 123.69, 117.42, 114.02, 113.84, 56.28, 55.83, 34.59, 30.60, 8.69.

ESI-TOF HRMS: m/z 395.2074 (C₂₂H₂₆N₄O₃+H⁺ requires 395.2077).

h. Preparation of A7

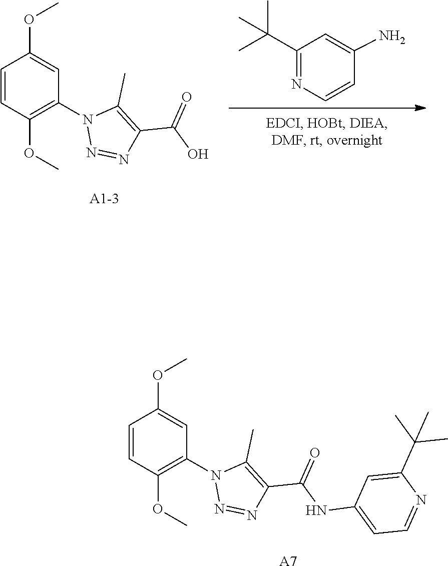 [SMILES: COc1ccc(OC)c(-n2nnc(C(=O)O)c2C)c1 ; CC(C)(C)c1cc(N)ccn1 ; COc1ccc(OC)c(-n2nnc(C(=O)Nc3ccnc(C(C)(C)C)c3)c2C)c1 ; "A7"]

The preparation of compound A7 was prepared by reaction of 1-(2,5-dimethoxyphenyl)-5-methyl-1H-1,2,3-triazole-4-carboxylic acid and 2-(tert-butyl)pyridin-4-amine according to the procedure for A1 synthesis. Light brown solid, 122.4 mg, 47% yield. ¹H NMR (400 MHz, Chloroform-d) δ 9.20 (s, 1H), 8.52 (d, J=5.5 Hz, 1H), 7.67 (d, J=2.0 Hz, 1H), 7.54 (dd, J=5.6, 2.0 Hz, 1H), 7.10 (dd, J=9.1, 3.0 Hz, 1H), 7.04 (d, J=9.1 Hz, 1H), 6.95 (d, J=3.0 Hz, 1H), 3.82 (s, 3H), 3.76 (s, 3H), 2.52 (s, 3H), 1.40 (s, 9H). ¹³C NMR (101 MHz, CDCl₃) δ 170.51, 159.86, 153.73, 149.36, 147.95, 145.31, 140.11, 137.40, 124.10, 117.60, 113.80, 113.34, 110.92, 109.15, 56.34, 56.02, 37.47, 30.12, 9.25. ESI-TOF HRMS: m/z 396.2038 (C₂₁H₂₅N₅O₃+H⁺ requires 396.2030).

i. Preparation of A8

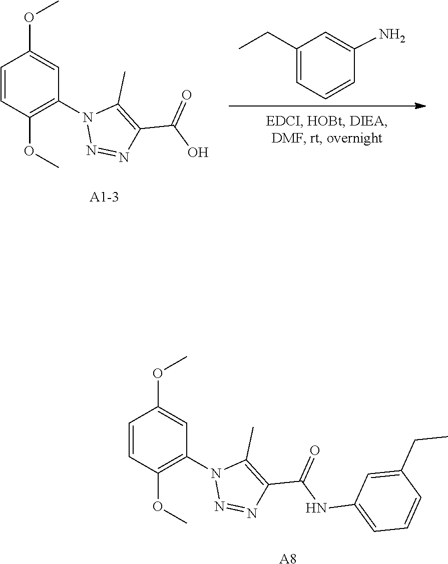

The preparation of compound A8 was prepared by reaction of 1-(2,5-dimethoxyphenyl)-5-methyl-1H-1,2,3-triazole-4-carboxylic acid and 3-ethylaniline according to the procedure for A1 synthesis. White solid, 209.9 mg, 57% yield. ¹H NMR (400 MHz, DMSO-d6) δ 10.36 (s, 1H), 7.76 (t, J=1.9 Hz, 1H), 7.70-7.64 (m, 1H), 7.31-7.19 (m, 3H), 7.15 (d, J=3.0 Hz, 1H), 6.95 (dt, J=7.7, 1.2 Hz, 1H), 3.78 (s, 3H), 3.75 (s, 3H), 2.61 (q, J=7.6 Hz, 2H), 2.39 (s, 3H), 1.20 (t, J=7.6 Hz, 3H). ¹³C NMR (101 MHz, DMSO) δ 159.41, 153.06, 147.69, 144.08, 138.93, 138.58, 137.59, 128.41, 123.72, 123.15, 119.77, 117.81, 117.35, 113.99, 113.84, 56.28, 55.84, 28.28, 15.47, 8.77. ESI-TOF HRMS: m/z 367.1762 (C₂₀H₂₂N₄O₃+H^{|} requires 367.1765).

j. Preparation of A9

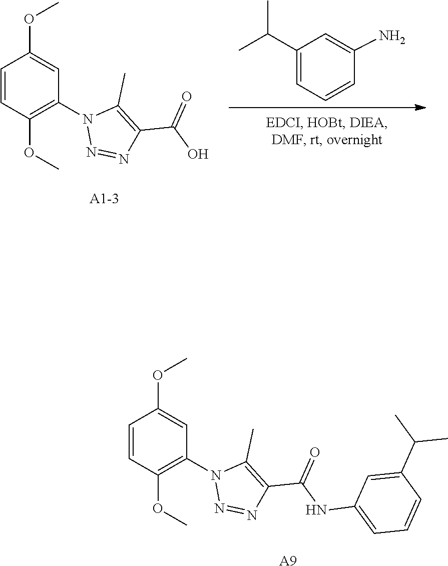

The preparation of compound A9 was prepared by reaction of 1-(2,5-dimethoxyphenyl)-5-methyl-1H-1,2,3-triazole-4-carboxylic acid and 3-isopropylaniline according to the procedure for A1 synthesis. White solid, 305.8 mg, 80% yield. ¹H NMR (400 MHz, DMSO-d6) δ 10.36 (s, 1H), 7.80 (t, J=2.0 Hz, 1H), 7.69 (ddd, J=8.1, 2.2, 1.1 Hz, 1H), 7.27 (dd, J=8.5, 6.8 Hz, 2H), 7.24-7.19 (m, 1H), 7.15 (d, J=3.0 Hz, 1H), 6.98 (dt, J=7.7, 1.4 Hz, 1H), 3.78 (s, 3H), 3.75 (s, 3H), 2.87 (p, J=6.9 Hz, 1H), 2.40 (s, 3H), 1.22 (d, J=6.9 Hz, 6H). ¹³C NMR (101 MHz, DMSO) δ 159.41, 153.07, 148.80, 147.68, 138.93, 138.59, 137.61, 128.39, 123.74, 121.71, 118.40, 117.91, 117.34, 113.99, 113.82, 56.27, 55.82, 33.50, 23.82, 8.77. ESI-TOF HRMS: m/z 381.1924 (C₂₁H₂₄N₄O₃+H⁺ requires 381.1921).

k. Preparation of A10

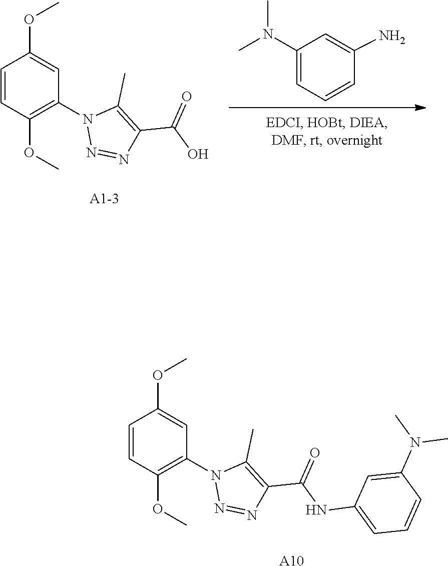

The preparation of compound A10 was prepared by reaction of 1-(2,5-dimethoxyphenyl)-5-methyl-1H-1,2,3-triazole-4-carboxylic acid and N1,N1-dimethylbenzene-1,3-diamine according to the procedure for A1 synthesis. Light brown solid, 301.5 mg, 79% yield. ¹H NMR (400 MHz, DMSO-d6) δ 10.18 (s, 1H), 7.32 (t, J=2.2 Hz, 1H), 7.29-7.24 (m, 2H), 7.21 (dd, J=9.1, 3.1 Hz, 1H), 7.16-7.09 (m, 2H), 6.49 (ddd, J=8.3, 2.6, 0.9 Hz, 1H), 3.78 (s, 3H), 3.75 (s, 3H), 2.90 (s, 6H), 2.39 (s, 3H). ¹³C NMR (101 MHz, DMSO) δ 159.31, 153.06, 150.76, 147.68, 139.33, 138.83, 137.70, 128.86, 123.74, 117.33, 113.99, 113.83, 108.47, 108.17, 104.54, 56.28, 55.83, 40.10, 8.78. ESI-TOF HRMS: m/z 382.1877 (C₂₀H₂₃N₅O₃+H^{|} requires 382.1874).

### 1. Preparation of A11

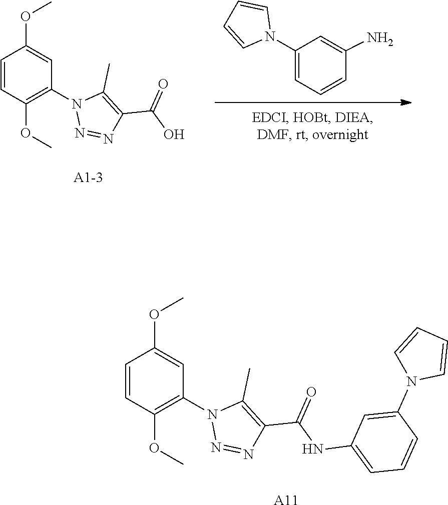 [SMILES: COc1ccc(OC)c(-n2nnc(C(=O)O)c2C)c1 ; Nc1cccc(-n2cccc2)c1 ; COc1ccc(OC)c(-n2nnc(C(=O)Nc3cccc(-n4cccc4)c3)c2C)c1 ; "A11"]

The preparation of compound A11 was prepared by reaction of 1-(2,5-dimethoxyphenyl)-5-methyl-1H-1,2,3-triazole-4-carboxylic acid and 3-(1H-pyrrol-1-yl)aniline according to the procedure for A1 synthesis. White solid, 352.2 mg, 69% yield. ¹H NMR (400 MHz, DMSO-d6) δ 10.63 (s, 1H), 8.15 (t, J=2.1 Hz, 1H), 7.84 (dd, J=8.4, 1.6 Hz, 1H), 7.43 (t, J=8.1 Hz, 1H), 7.31 (q, J=3.8, 2.9 Hz, 3H), 7.27 (d, J=9.1 Hz, 1H), 7.21 (dd, J=9.1, 3.0 Hz, 1H), 7.17 (d, J=3.0 Hz, 1H), 6.29 (t, J=2.2 Hz, 2H), 3.78 (s, 3H), 3.75 (s, 3H), 2.42 (s, 3H). ¹³C NMR (101 MHz, DMSO) δ 159.68, 153.08, 147.67, 140.15, 139.86, 139.27, 137.41, 129.81, 123.69, 118.86, 117.34, 117.09, 114.63, 114.01, 113.80, 111.39, 110.48, 56.26, 55.81, 8.80. ESI-TOF HRMS: m/z 404.1716 (C₂₂H₂₁N₅O₃+H⁺ requires 404.1717).

m. Preparation of A12

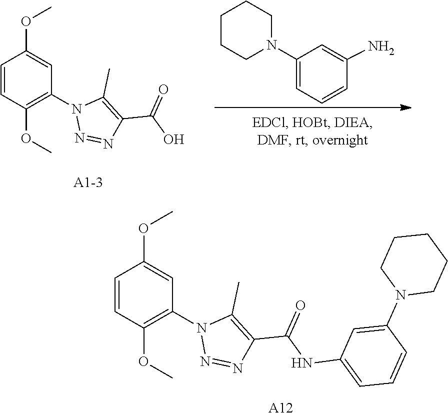 [SMILES: COc1ccc(OC)c(-n2nnc(C(=O)O)c2C)c1 ; Nc1cccc(N2CCCCC2)c1 ; COc1ccc(OC)c(-n2nnc(C(=O)Nc3cccc(N4CCCCC4)c3)c2C)c1 ; "A12"]

The preparation of compound A12 was prepared by reaction of 1-(2,5-dimethoxyphenyl)-5-methyl-1H-1,2,3-triazole-4-carboxylic acid and 3-(piperidin-1-yl)aniline according to the procedure for A1 synthesis. Light brown solid, 316 mg, 75% yield. ¹H NMR (400 MHz, Chloroform-d) δ 9.03 (s, 1H), 7.46 (s, 1H), 7.23 (t, J=8.1 Hz, 1H), 7.08 (dq, J=5.9, 2.9 Hz, 2H), 7.03 (d, J=9.1 Hz, 1H), 6.95 (d, J=3.0 Hz, 1H), 6.74 (s, 1H), 3.82 (s, 3H), 3.76 (s, 3H), 3.21 (t, J=5.4 Hz, 4H), 2.52 (s, 3H), 1.77-1.66 (m, 4H), 1.59 (q, J=5.8 Hz, 2H). ¹³C NMR (101 MHz, CDCl3) δ 159.36, 153.70, 148.04, 139.36, 138.68, 138.02, 129.46, 124.36, 117.49, 113.81, 113.34, 112.46, 110.42, 107.68, 56.34, 56.01, 50.51, 25.78, 24.32, 9.25. ESI-TOF HRMS: m/z 422.2190 (C₂₃H₂₇N₅O₃+H⁺ requires 422.2187).

n. Preparation of A13

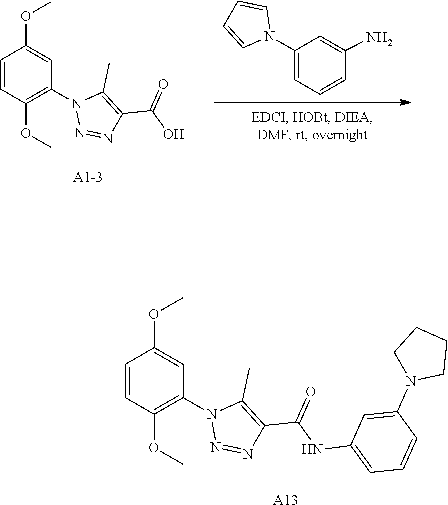 [SMILES: COc1ccc(OC)c(-n2nnc(C(=O)O)c2C)c1 ; Nc1cccc(-n2cccc2)c1 ; COc1ccc(OC)c(-n2nnc(C(=O)Nc3cccc(N4CCCC4)c3)c2C)c1 ; "A13"]

The preparation of compound A13 was prepared by reaction of 1-(2,5-dimethoxyphenyl)-5-methyl-1H-1,2,3-triazole-4-carboxylic acid and 3-(piperidin-1-yl)aniline according to the procedure for A1 synthesis. White solid, 341.0 mg, 84% yield. ¹H NMR (400 MHz, Chloroform-d) δ 9.03 (s, 1H), 7.19 (t, J=8.1 Hz, 1H), 7.06 (dd, J=9.1, 2.9 Hz, 2H), 7.00 (d, J=9.2 Hz, 1H), 6.98-6.92 (m, 2H), 6.40-6.33 (m, 1H), 3.79 (s, 3H), 3.73 (s, 3H), 3.37-3.29 (m, 4H), 2.55-2.45 (m, 3H), 2.02-1.95 (m, 4H). ¹³C NMR (101 MHz, CDCl₃) δ 159.32, 153.67, 148.51, 148.01, 139.32, 138.78, 138.11, 129.58, 124.35, 117.40, 113.86, 113.35, 108.01, 107.09, 103.08, 56.32, 55.99, 47.82, 25.45, 9.24. ESI-TOF HRMS: m/z 408.2026 (C₂₂H₂₅N₅O₃+H⁺ requires 408.2030).

o. Preparation of A14

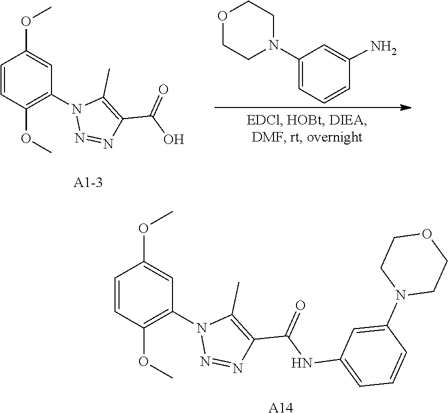 [SMILES: COc1ccc(OC)c(-n2nnc(C(=O)O)c2C)c1 ; Nc1cccc(N2CCOCC2)c1 ; COc1ccc(OC)c(-n2nnc(C(=O)Nc3cccc(N4CCOCC4)c3)c2C)c1 ; "A14"]

The preparation of compound A14 was prepared by reaction of 1-(2,5-dimethoxyphenyl)-5-methyl-1H-1,2,3-triazole-4-carboxylic acid and 3-morpholinoaniline according to the procedure for A1 synthesis. White solid, 248.2 mg, 59% yield. ¹H NMR (400 MHz, DMSO-d6) δ 10.26 (s, 1H), 7.54 (t, J=2.2 Hz, 1H), 7.40-7.35 (m, 1H), 7.28 (d, J=9.2 Hz, 1H), 7.24-7.16 (m, 2H), 7.15 (d, J=3.0 Hz, 1H), 6.70 (ddd, J=8.3, 2.5, 0.9 Hz, 1H), 3.78 (s, 3H), 3.77-3.73 (m, 7H), 3.13-3.08 (m, 4H), 2.38 (s, 3H). ¹³C NMR (101 MHz, DMSO) δ 159.38, 153.06, 151.37, 147.68, 139.40, 138.92, 137.59, 128.96, 123.71, 117.36, 113.99, 113.84, 111.29, 110.73, 107.12, 66.06, 56.29, 55.84, 48.48, 8.78. ESI-TOF HRMS: m/z 424.1982 (C₂₂H₂₅N₅O₄+H⁺ requires 424.1980).

p. Preparation of A15

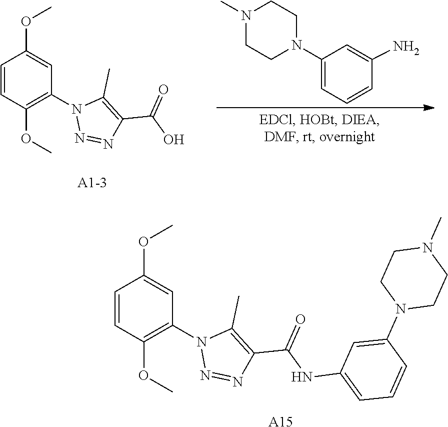 [SMILES: COc1ccc(OC)c(-n2nnc(C(=O)O)c2C)c1 ; CN1CCN(c2cccc(N)c2)CC1 ; COc1ccc(OC)c(-n2nnc(C(=O)Nc3cccc(N4CCN(C)CC4)c3)c2C)c1 ; "A15"]

The preparation of compound A15 was prepared by reaction of 1-(2,5-dimethoxyphenyl)-5-methyl-1H-1,2,3-triazole-4-carboxylic acid and 3-(4-methylpiperazin-1-yl)aniline according to the procedure for A1 synthesis. Light brown solid, 348.5 mg, 80% yield. ¹H NMR (400 MHz, DMSO-d6) δ 10.23 (s, 1H), 7.53 (t, J=2.2 Hz, 1H), 7.35 (dd, J=7.9, 1.8 Hz, 1H), 7.28 (d, J=9.2 Hz, 1H), 7.22 (dd, J=9.1, 3.0 Hz, 1H), 7.19-7.13 (m, 2H), 6.69 (dd, J=8.3, 2.4 Hz, 1H), 3.78 (s, 3H), 3.75 (s, 3H), 3.13 (t, J=4.8 Hz, 4H), 2.46 (t, J=4.9 Hz, 4H), 2.39 (s, 3H), 2.22 (s, 3H). ¹³C NMR (101 MHz, DMSO) δ 159.35, 153.06, 151.28, 147.68, 139.35, 138.89, 137.61, 128.91, 123.72, 117.35, 113.99, 113.84, 110.93, 110.90, 107.35, 56.29, 55.84, 54.56, 48.06, 45.73, 8.78. ESI-TOF HRMS: m/z 437.2295 (C₂₃H₂₈N₆O₃+H⁺ requires 437.2296).

q. Preparation of A16

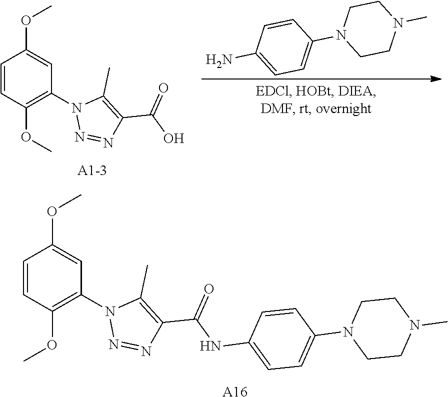 [SMILES: COc1ccc(OC)c(-n2nnc(C(=O)O)c2C)c1 ; CN1CCN(c2ccc(N)cc2)CC1 ; COc1ccc(OC)c(-n2nnc(C(=O)Nc3ccc(N4CCN(C)CC4)cc3)c2C)c1 ; "A16"]

The preparation of compound A16 was prepared by reaction of 1-(2,5-dimethoxyphenyl)-5-methyl-1H-1,2,3-triazole-4-carboxylic acid and 4-(4-methylpiperazin-1-yl)anilin according to the procedure for A1 synthesis. White solid, 329.8 mg, 75% yield. ¹H NMR (400 MHz, DMSO-d6) δ 10.25 (s, 1H), 7.72-7.64 (m, 2H), 7.27 (d, J=9.2 Hz, 1H), 7.21 (dd, J=9.1, 3.0 Hz, 1H), 7.13 (d, J=3.0 Hz, 1H), 6.94-6.90 (m, 2H), 3.78 (s, 3H), 3.75 (s, 3H), 3.10 (dd, J=6.2, 3.8 Hz, 4H), 2.46 (t, J=5.0 Hz, 4H), 2.38 (s, 3H), 2.22 (s, 3H). ¹³C NMR (101 MHz, DMSO) δ 158.97, 153.05, 147.69, 147.51, 138.58, 137.72, 130.46, 123.78, 121.46, 117.30, 115.43, 114.00, 113.83, 56.28, 55.83, 54.59, 48.45, 45.71, 8.75. ESI-TOF HRMS: m/z 437.2294 (C₂₃H₂₈N₆O₃+H⁺ requires 437.2296).

r. Preparation of A17

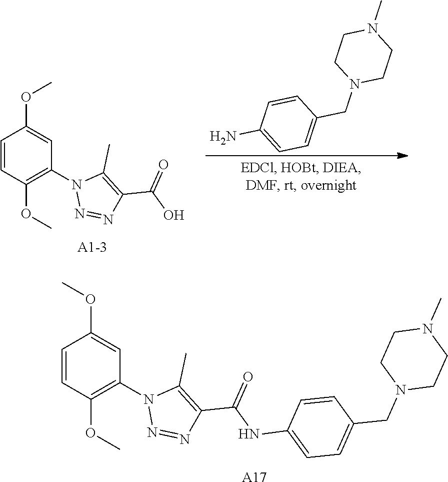

The preparation of compound A17 was prepared by reaction of 1-(2,5-dimethoxyphenyl)-5-methyl-1H-1,2,3-triazole-4-carboxylic acid and 4-((4-methylpiperazin-1-yl)methyl)aniline according to the procedure for A1 synthesis. Light brown solid, 311.2 mg, 69% yield. ¹H NMR (400 MHz, DMSO-d₆) δ 10.43 (s, 1H), 7.82-7.75 (m, 2H), 7.32-7.18 (m, 4H), 7.14 (d, J=3.0 Hz, 1H), 3.78 (s, 3H), 3.75 (s, 3H), 3.42 (s, 2H), 2.38 (s, 3H), 2.38-2.27 (m, 8H), 2.16 (s, 3H). ¹³C NMR (101 MHz, DMSO-d₆) δ 159.38, 153.05, 147.69, 138.93, 137.55, 137.36, 133.48, 129.00, 123.71, 120.24, 117.37, 114.00, 113.86, 61.62, 56.30, 55.85, 54.66, 52.39, 45.62, 8.77. ESI-TOF HRMS: m/z 451.2454 (C₂₄H₃₀N₆O₃+H⁺ requires 451.2452).

s. Preparation of A18

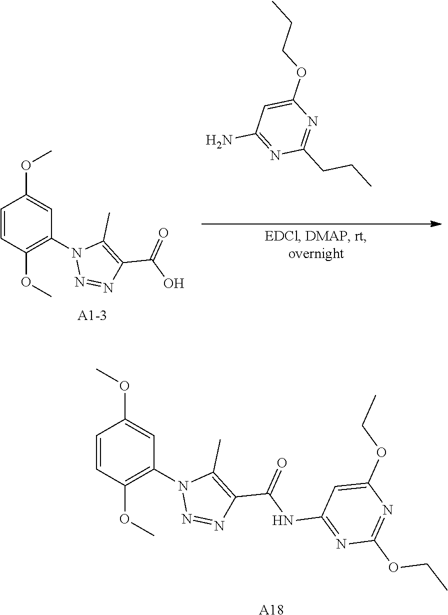 [SMILES: COc1ccc(OC)c(-n2nnc(C(=O)O)c2C)c1 ; CCCOc1cc(N)nc(CCC)n1 ; CCOc1cc(NC(=O)c2nnn(-c3cc(OC)ccc3OC)c2C)nc(OCC)n1 ; "A18"]

To a solution of 1-(2,5-dimethoxyphenyl)-5-methyl-1H-1,2,3-triazole-4-carboxylic acid (100 mg, 0.380 mmol) in DMF 5 mL was added 2,6-diethoxypyrimidin-4-amine (139 mg, 0.760 mmol), EDCI (146 mg, 0.760 mmol) and N,N-dimethylpyridin-4-amine (46.4 mg, 0.380 mmol). The reaction mixture was stirred at room temperature for overnight. The reaction mixture was then diluted with water (50 mL) and extracted with EtOAc (50 mL×2). The combine organic layers were washed with water (50 mL) and brine (50 mL), dried with anhydrous Na₂SO₄, and concentrated. The residue was purified by silica gel chromatography (0% to 100% acetonitrile in water) to give product as white solid (93.8 mg, 58% yield). ¹H NMR (400 MHz, Chloroform-d) δ 9.53 (s, 1H), 7.31 (s, 1H), 7.09 (dd, J=9.1, 3.0 Hz, 1H), 7.03 (d, J=9.2 Hz, 1H), 6.94 (d, J=3.0 Hz, 1H), 4.40 (dq, J=10.2, 7.0 Hz, 4H), 3.82 (s, 3H), 3.76 (s, 3H), 2.50 (s, 3H), 1.40 (dt, J=13.8, 7.1 Hz, 6H). ¹³C NMR (101 MHz, CDCl₃) δ 172.97, 164.23, 160.03, 158.47, 153.72, 147.94, 140.32, 137.22, 124.15, 117.62, 113.74, 113.36, 88.68, 63.40, 62.77, 56.35, 56.02, 14.51, 14.45, 9.26. ESI-TOF HRMS: m/z 429.1880 (C₂₀H₂₄N₆O₅+H⁺ requires 429.1881).

t. Preparation of A19

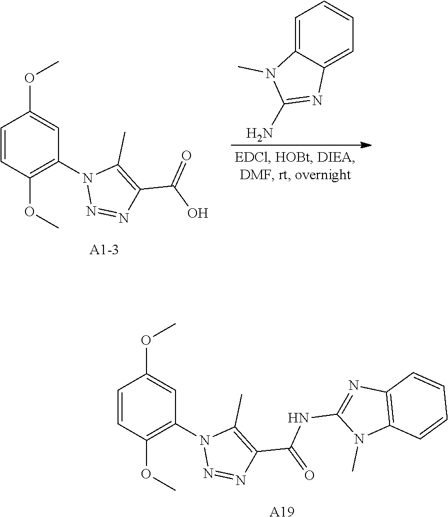 [SMILES: COc1ccc(OC)c(-n2nnc(C(=O)O)c2C)c1 ; Cn1c(N)nc2ccccc21 ; COc1ccc(OC)c(-n2nnc(C(=O)Nc3nc4ccccc4n3C)c2C)c1 ; "A19"]

The preparation of compound A19 was prepared by reaction of 1-(2,5-dimethoxyphenyl)-5-methyl-1H-1,2,3-triazole-4-carboxylic acid and 1-methyl-1H-benzo[d]imidazol-2-amine according to the procedure for A1 synthesis. Light brown solid, 261 mg, 67% yield. ¹H NMR (400 MHz, Chloroform-d) δ 7.41-7.34 (m, 1H), 7.33-7.21 (m, 3H), 7.05 (dd, J=9.1, 2.9 Hz, 1H), 7.00 (d, J=9.1 Hz, 1H), 6.95 (d, J=2.9 Hz, 1H), 3.80 (s, 3H), 3.79 (s, 3H), 3.73 (s, 3H), 2.56 (s, 3H). ¹³C NMR (101 MHz, CDCl₃) δ 153.69, 148.22, 142.01, 139.04, 130.49, 125.52, 125.11, 123.22, 123.19, 117.15, 113.99, 113.52, 111.77, 109.94, 109.18, 56.47, 55.99, 28.77, 10.04. ESI-TOF HRMS: m/z 393.1671 (C₂₀H₂₀N₆O₃+H⁺ requires 393.1670).

u. Preparation of A20

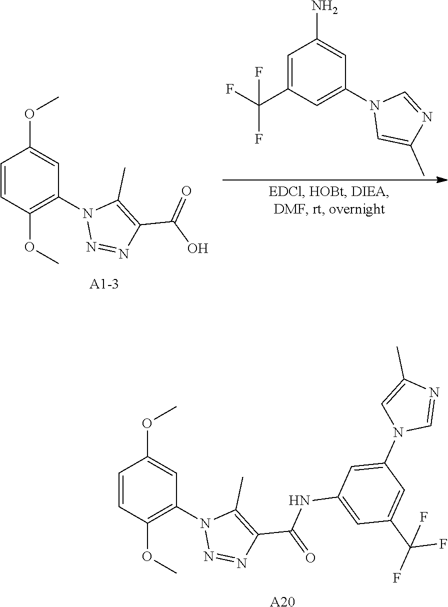 [SMILES: COc1ccc(OC)c(-n2nnc(C(=O)O)c2C)c1 ; Cc1cn(-c2cc(N)cc(C(F)(F)F)c2)cn1 ; COc1ccc(OC)c(-n2nnc(C(=O)Nc3cc(-n4cnc(C)c4)cc(C(F)(F)F)c3)c2C)c1 ; "A20"]

The preparation of compound A20 was prepared by reaction of 1-(2,5-dimethoxyphenyl)-5-methyl-1H-1,2,3-triazole-4-carboxylic acid and 3-(4-methyl-1H-imidazol-1-yl)-5-(trifluoromethyl)aniline according to the procedure for A1 synthesis. White solid, 722.9 mg, 74% yield. ¹H NMR (400 MHz, DMSO-d6) δ 10.98 (s, 1H), 8.37 (d, J=16.4 Hz, 2H), 8.21 (s, 1H), 7.73 (s, 1H), 7.49 (s, 1H), 7.32-7.12 (m, 3H), 3.78 (s, 3H), 3.76 (s, 3H), 2.41 (s, 3H), 2.19 (s, 3H). ¹³C NMR (101 MHz, DMSO) δ 160.03, 153.07, 147.65, 140.84, 139.74, 138.88, 137.84, 136.94, 134.88, 131.00, 130.68, 130.36, 125.00, 123.54, 122.29, 117.48, 114.89, 114.09, 113.97, 113.86, 111.43, 56.31, 55.85, 13.52, 8.83. ESI-TOF HRMS: m/z 487.1697 (C₂₃H₂₁F₃N₆O₃+H⁺ requires 487.1700).

v. Preparation of A21

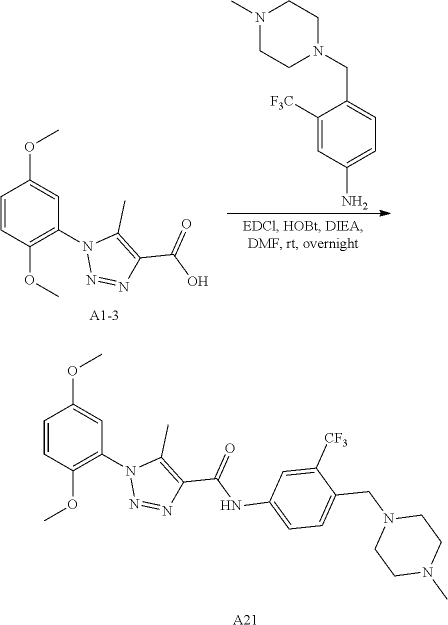

The preparation of compound A21 was prepared by reaction of 1-(2,5-dimethoxyphenyl)-5-methyl-1H-1,2,3-triazole-4-carboxylic acid and 4-((4-methylpiperazin-1-yl)methyl)-3-(trifluoromethyl)aniline (0.273 g, 1.000 mmol) according to the procedure for A1 synthesis. Light brown solid, 398.5 mg, 77% yield. ¹H NMR (400 MHz, DMSO-d6) δ 10.83 (s, 1H), 8.36 (d, J=2.2 Hz, 1H), 8.10 (dd, J=8.5, 2.3 Hz, 1H), 7.68 (d, J=8.5 Hz, 1H), 7.28 (d, J=9.2 Hz, 1H), 7.22 (dd, J=9.1, 3.0 Hz, 1H), 7.15 (d, J=3.0 Hz, 1H), 3.78 (s, 3H), 3.75 (s, 3H), 3.57 (s, 2H), 2.47-2.34 (m, 11H), 2.20 (s, 3H). ¹³C NMR (101 MHz, DMSO) δ 159.80, 153.06, 147.66, 139.35, 137.78, 137.23, 131.91, 131.07, 128.40, 127.81, 127.51, 127.22, 126.92, 125.67, 123.63, 122.95, 120.22, 117.39, 117.31, 113.97, 113.84, 57.35, 56.28, 55.82, 54.52, 52.38, 45.35, 8.79. ESI-TOF HRMS: m/z 519.2333 (C₂₅H₂₉F₃N₆O₃+H⁺ requires 519.2326).

w. Preparation of A22

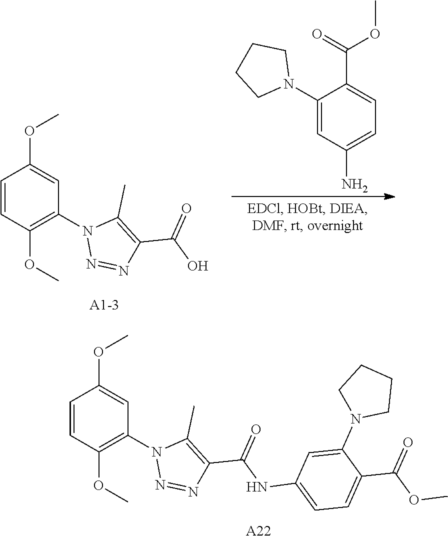

The preparation of compound A22 was prepared by reaction of 1-(2,5-dimethoxyphenyl)-5-methyl-1H-1,2,3-triazole-4-carboxylic acid and 4-amino-2-(pyrrolidin-1-yl)benzoate according to the procedure for A1 synthesis. White solid, 2.92 g, 66% yield. ¹H NMR (400 MHz, Chloroform-d) δ 9.12 (s, 1H), 7.63 (d, J=8.5 Hz, 1H), 7.41 (s, 1H), 7.08 (dd, J=9.1, 3.0 Hz, 1H), 7.02 (d, J=9.1 Hz, 2H), 6.94 (d, J=3.0 Hz, 1H), 3.87 (s, 3H), 3.80 (s, 3H), 3.75 (s, 3H), 3.29 (d, J=6.3 Hz, 4H), 2.50 (s, 3H), 1.98-1.92 (m, 4H). ¹³C NMR (101 MHz, CDCl3) δ 168.62, 159.50, 153.69, 149.08, 147.99, 141.19, 139.63, 137.83, 132.55, 124.25, 117.50, 113.81, 113.34, 113.12, 107.49, 104.60, 56.33, 56.00, 51.91, 51.36, 25.86, 9.25. ESI-TOF HRMS: m/z 466.2085 (C₂₄H₂₇N₅O₅+H⁺ requires 466.2085).

x. Preparation of A23

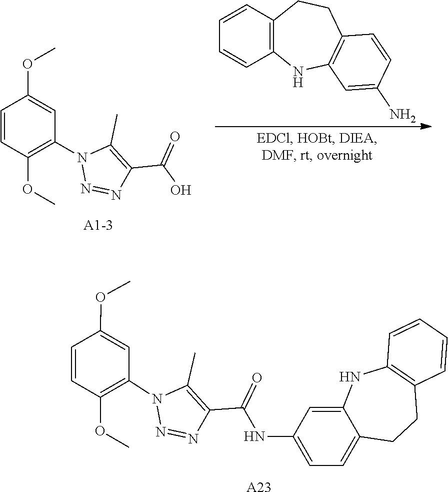 [SMILES: COc1ccc(OC)c(-n2nnc(C(=O)O)c2C)c1 ; Nc1ccc2c(c1)Nc1ccccc1CC2 ; COc1ccc(OC)c(-n2nnc(C(=O)Nc3ccc4c(c3)Nc3ccccc3CC4)c2C)c1 ; "A23"]

The preparation of compound A23 was prepared by reaction of 1-(2,5-dimethoxyphenyl)-5-methyl-1H-1,2,3-triazole-4-carboxylic acid and 10,11-dihydro-5H-dibenzo[b,f]azepin-3-amine according to the procedure for A1 synthesis. White solid, 2.08 g, 79% yield. ¹H NMR (400 MHz, Chloroform-d) δ 9.01 (s, 1H), 7.56 (d, J=2.1 Hz, 1H), 7.12-6.99 (m, 5H), 6.95 (d, J=3.0 Hz, 1H), 6.84-6.75 (m, 2H), 6.73 (dd, J=8.0, 1.2 Hz, 1H), 6.18 (s, 1H), 3.82 (s, 3H), 3.77 (s, 3H), 3.12-3.03 (m, 4H), 2.52 (s, 3H). ¹³C NMR (101 MHz, CDCl3) δ 159.40, 153.71, 148.03, 142.89, 142.38, 139.47, 137.90, 136.61, 131.17, 130.60, 128.88, 126.87, 124.51, 124.33, 119.65, 118.07, 117.52, 113.82, 113.34, 110.59, 109.05, 56.35, 56.01, 34.99, 34.62, 9.23. ESI-TOF HRMS: m/z 456.2030 (C₂₆H₂₅N₅O₃+H⁺ requires 456.2030).

y. Preparation of A24

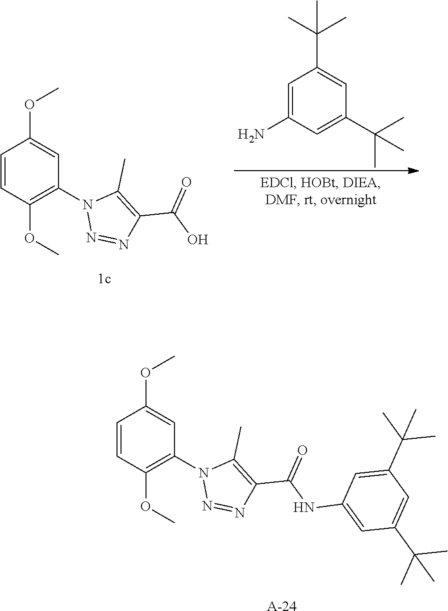 [SMILES: COc1ccc(OC)c(-n2nnc(C(=O)O)c2C)c1 ; CC(C)(C)c1cc(N)cc(C(C)(C)C)c1 ; COc1ccc(OC)c(-n2nnc(C(=O)Nc3cc(C(C)(C)C)cc(C(C)(C)C)c3)c2C)c1 ; "A-24"]

The preparation of compound A24 was prepared by reaction of 1-(2,5-dimethoxyphenyl)-5-methyl-1H-1,2,3-triazole-4-carboxylic acid and 3,5-di-tert-butylaniline according to the procedure for A1 synthesis. White solid, 177.2 mg, 79% yield. ¹H NMR (400 MHz, Chloroform-d) δ 9.08 (s, 1H), 7.59 (d, J=1.7 Hz, 2H), 7.22 (t, J=1.7 Hz, 1H), 7.09 (dd, J=9.1, 3.0 Hz, 1H), 7.03 (d, J=9.1 Hz, 1H), 6.96 (d, J=2.9 Hz, 1H), 3.82 (s, 3H), 3.76 (s, 3H), 2.53 (s, 3H), 1.36 (s, 18H). ¹³C NMR (101 MHz, CDCl3) δ 159.32, 153.71, 151.71, 148.05, 139.29, 137.26, 124.40, 118.43, 117.48, 114.38, 113.84, 113.35, 56.34, 56.01, 35.00, 31.47, 9.28. ESI-TOF HRMS: m/z 451.2695 (C₂₆H₃₄N₄O₃+H⁺ requires 451.2704).

z. Preparation of A25

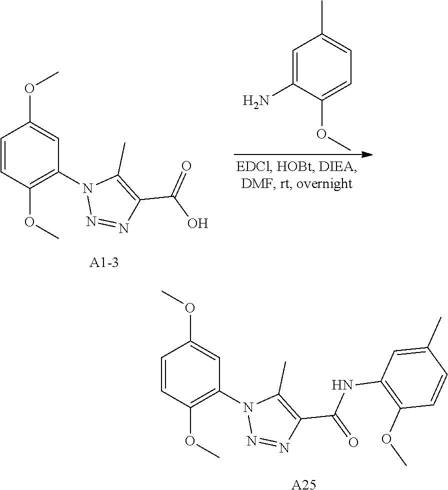

The preparation of compound A25 was prepared by reaction of 1-(2,5-dimethoxyphenyl)-5-methyl-1H-1,2,3-triazole-4-carboxylic acid and 2-methoxy-5-methylaniline according to the procedure for A1 synthesis. White solid, 122.9 mg, 85% yield. ¹H NMR (500 MHz, DMSO-d6) δ 9.54 (s, 1H), 8.15 (d, J=2.1 Hz, 1H), 7.29 (d, J=9.2 Hz, 1H), 7.23 (dd, J=9.2, 3.1 Hz, 1H), 7.15 (d, J=3.0 Hz, 1H), 7.01 (d, J=8.3 Hz, 1H), 6.93 (ddd, J=8.4, 2.2, 0.9 Hz, 1H), 3.90 (s, 3H), 3.78 (s, 3H), 3.75 (s, 3H), 2.40 (s, 3H), 2.28 (s, 3H). ¹³C NMR (126 MHz, DMSO) δ 158.89, 153.54, 148.15, 146.86, 139.42, 137.65, 129.88, 127.06, 124.81, 124.05, 120.59, 117.95, 114.51, 114.40, 111.32, 56.83, 56.56, 56.35, 21.08, 9.19. ESI-TOF HRMS: m/z 383.1719 (C₂₀H₂₂N₄O₄+H⁺ requires 383.1714).

aa. Preparation of B1

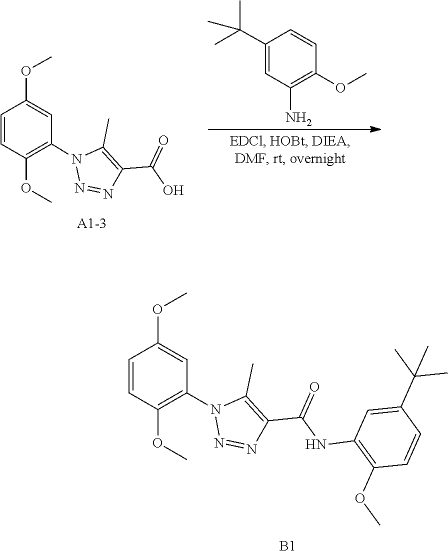 [SMILES: COc1ccc(OC)c(-n2nnc(C(=O)O)c2C)c1 ; COc1ccc(C(C)(C)C)cc1N ; COc1ccc(OC)c(-n2nnc(C(=O)Nc3cc(C(C)(C)C)ccc3OC)c2C)c1 ; "B1"]

The preparation of compound B1 was prepared by reaction of 1-(2,5-dimethoxyphenyl)-5-methyl-1H-1,2,3-triazole-4-carboxylic acid and 5-(tert-butyl)-2-methoxyaniline according to the procedure for A1 synthesis. White solid, 355.9 mg, 84% yield. ¹H NMR (400 MHz, DMSO-d6) δ 9.56 (s, 1H), 8.41 (d, J=2.4 Hz, 1H), 7.29 (d, J=9.2 Hz, 1H), 7.23 (dd, J=9.1, 3.0 Hz, 1H), 7.17-7.09 (m, 2H), 7.03 (d, J=8.6 Hz, 1H), 5.75 (s, 1H), 3.91 (s, 3H), 3.78 (s, 3H), 3.76 (s, 3H), 2.41 (s, 3H), 1.29 (s, 9H). ¹³C NMR (101 MHz, DMSO) δ 158.47, 153.06, 147.65, 146.31, 142.91, 138.90, 137.20, 126.36, 123.58, 120.65, 117.44, 116.78, 113.99, 113.91, 110.48, 56.33, 56.04, 55.85, 34.00, 31.31, 8.72. ESI-TOF HRMS: m/z 425.2189 (C₂₃H₂₈N₄O₄+H⁺ requires 425.2184).

bb. Preparation of B2 and B3

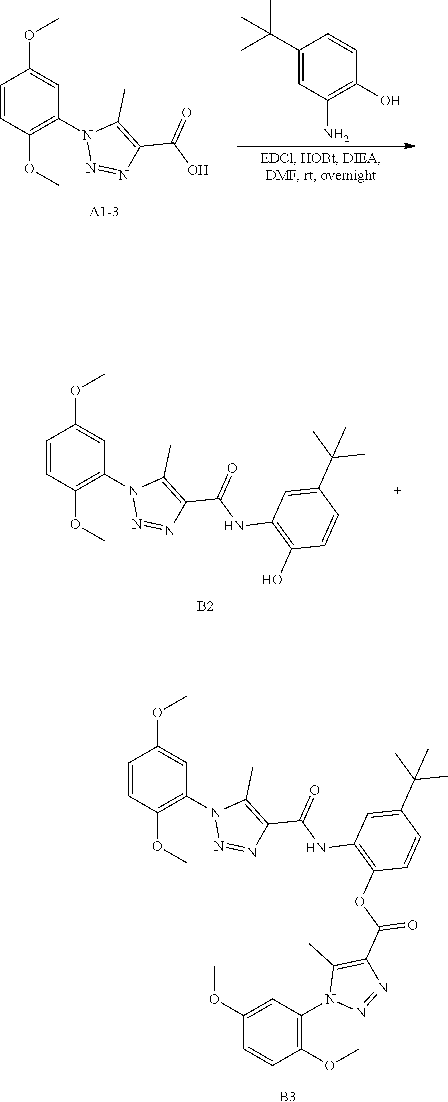

The preparation of compound B2 and B3 was prepared by reaction of 1-(2,5-dimethoxyphenyl)-5-methyl-1H-1,2,3-triazole-4-carboxylic acid and 2-amino-4-(tert-butyl)phenol according to the procedure for A1 synthesis.

N-(5-(tert-butyl)-2-hydroxyphenyl)-1-(2,5-dimethoxyphenyl)-5-methyl-1H-1,2,3-triazole-4-carboxamide (181.8 mg, 56% yield) as a white solid. ¹H NMR (400 MHz, DMSO-d₆) δ 10.01 (s, 1H), 9.61 (s, 1H), 8.33 (d, J=2.4 Hz, 1H), 7.29 (d, J=9.2 Hz, 1H), 7.22 (dd, J=9.1, 3.0 Hz, 1H), 7.16 (d, J=3.0 Hz, 1H), 6.99 (dd, J=8.4, 2.4 Hz, 1H), 6.86 (d, J=8.5 Hz, 1H), 3.78 (s, 3H), 3.76 (s, 3H), 2.41 (s, 3H), 1.27 (s, 9H). ¹³C NMR (101 MHz, DMSO) δ 158.45, 153.06, 147.65, 144.15, 141.51, 138.80, 137.30, 125.64, 123.61, 120.65, 117.44, 116.80, 114.28, 113.98, 113.89, 56.32, 55.85, 33.91, 31.39, 8.72. ESI-TOF HRMS: m/z 411.2025 (C₂₂H₂₆N₄O₄+H⁺ requires 411.2027).

4-(tert-butyl)-2-(1-(2,5-dimethoxyphenyl)-5-methyl-1H-1,2,3-triazole-4-carboxamido)phenyl 1-(2,5-dimethoxyphenyl)-5-methyl-1H-1,2,3-triazole-4-carboxylate (78.5 mg, 24% yield) as a white solid. ¹H NMR (400 MHz, DMSO-d₆) δ 9.97 (s, 1H), 8.00 (d, J=2.1 Hz, 1H), 7.37 (d, J=8.5 Hz, 1H), 7.34 (dd, J=8.6, 2.2 Hz, 1H), 7.28 (t, J=9.1 Hz, 2H), 7.22 (dd, J=6.3, 3.0 Hz, 1H), 7.21-7.18 (m, OH), 7.12 (dd, J=5.0, 3.0 Hz, 2H), 3.76 (s, 3H), 3.75 (s, 5H), 3.73 (s, 3H), 3.69 (s, 3H), 2.42 (s, 3H), 2.31 (s, 3H), 1.35 (s, 9H). ¹³C NMR (101 MHz, DMSO-d₆) δ 159.17, 158.85, 153.07, 153.04, 148.65, 147.59, 141.50, 139.81, 138.98, 137.18, 134.69, 128.99, 123.58, 123.40, 122.68, 122.31, 121.55, 117.59, 117.41, 113.95, 113.89, 113.84, 56.25, 56.22, 55.82, 34.39, 31.12, 18.52, 9.37, 8.64. ESI-TOF HRMS: m/z 656.2833 (C₃₄H₃₇N₇O₇+H⁺ requires 656.2827).

cc. Preparation of B4

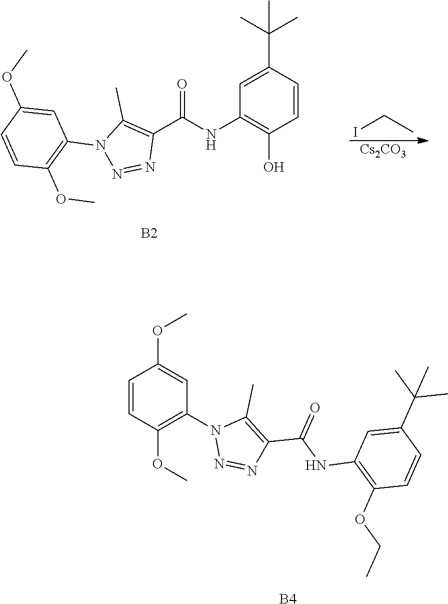

To a solution of N-(5-(tert-butyl)-2-hydroxyphenyl)-1-(2,5-dimethoxyphenyl)-5-methyl-1H-1,2,3-triazole-4-carboxamide (100 mg, 0.244 mmol) in DMF 5 mL at room temperature was added Cs₂CO₃ (95 mg, 0.292 mmol) and iodoethane (45.6 mg, 0.292 mmol). The suspension was stirred at 60° C. for overnight. The reaction mixture was then diluted with water (50 mL) and extracted with EA (50 mL×2). The EA layer was washed with water, dried with anhydrous Na₂SO₄, and concentrated with a Rotavapor. The residue was purified by silica gel chromatography (0% to 100% EA in hexane) to give product (87.6 mg, 82% yield, white solid). ¹H NMR (500 MHz, DMSO-d6) δ 9.61 (s, 1H), 8.44 (d, J=2.4 Hz, 1H), 7.29 (d, J=9.2 Hz, 1H), 7.23 (dd, J=9.2, 3.1 Hz, 1H), 7.16 (d, J=3.0 Hz, 1H), 7.10 (dd, J=8.6, 2.4 Hz, 1H), 7.03 (d, J=8.6 Hz, 1H), 4.16 (q, J=6.9 Hz, 2H), 3.78 (s, 3H), 3.76 (s, 3H), 2.41 (s, 3H), 1.41 (t, J=6.9 Hz, 3H), 1.29 (s, 9H). ¹³C NMR (126 MHz, DMSO) δ 158.91, 153.55, 148.12, 145.79, 143.44, 139.40, 137.76, 127.18, 124.07, 121.04, 117.95, 116.96, 114.46, 114.40, 112.11, 64.77, 56.83, 56.34, 34.52, 31.81, 15.22, 9.24. ESI-TOF HRMS: m/z 439.2329 (C₂₄H₃₀N₄O₄+H⁺ requires 439.2340).

dd. Preparation of B5

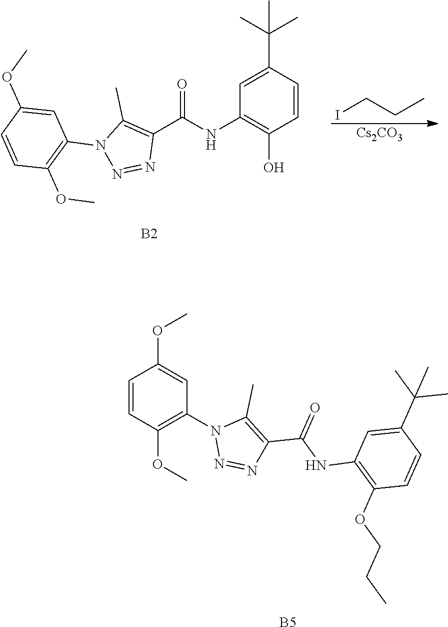

The preparation of compound B5 was prepared by reaction of N-(5-(tert-butyl)-2-hydroxyphenyl)-1-(2,5-dimethoxyphenyl)-5-methyl-1H-1,2,3-triazole-4-carboxamide and 1-iodopropane according to the procedure for B4 synthesis. 99.4 mg, 90% yield, white solid. ¹H NMR (500 MHz, DMSO-d₆) δ 9.66 (s, 1H), 8.46 (d, J=2.4 Hz, 1H), 7.29 (d, J=9.2 Hz, 1H), 7.22 (dd, J=9.1, 3.1 Hz, 1H), 7.17 (d, J=3.1 Hz, 1H), 7.10 (dd, J=8.6, 2.4 Hz, 1H), 7.02 (d, J=8.6 Hz, 1H), 4.07 (t, J=6.3 Hz, 2H), 3.78 (s, 3H), 3.76 (s, 3H), 2.41 (s, 3H), 1.81 (h, J=7.4 Hz, 2H), 1.29 (s, 9H), 1.07 (t, J=7.4 Hz, 3H). ¹³C NMR (126 MHz, DMSO) δ 158.89, 153.55, 148.10, 145.82, 143.43, 139.39, 137.77, 127.25, 124.06, 120.97, 117.98, 116.76, 114.44, 114.38, 111.99, 70.39, 56.82, 56.34, 34.53, 31.82, 22.62, 10.91, 9.23. ESI-TOF HRMS: m/z 453.2500 (C₂₅H₃₂N₄O₄+H⁺ requires 453.2496).

ee. Preparation of B6

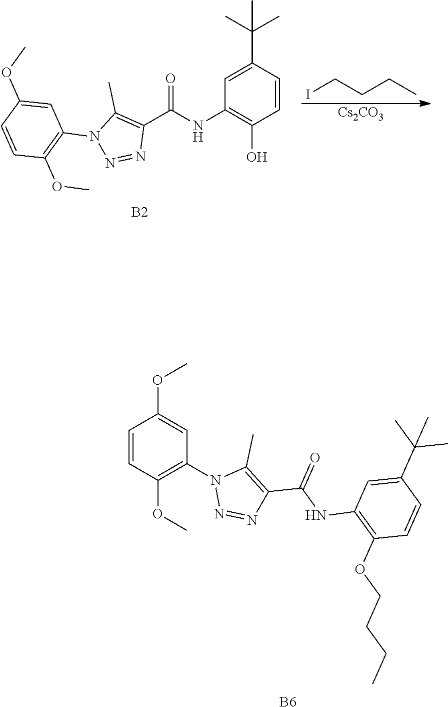

The preparation of compound B6 was prepared by reaction of N-(5-(tert-butyl)-2-hydroxyphenyl)-1-(2,5-dimethoxyphenyl)-5-methyl-1H-1,2,3-triazole-4-carboxamide and 1-iodobutane according to the procedure for B4 synthesis. 391.8 mg, 84% yield, white solid. ¹H NMR (400 MHz, DMSO-d6) δ 9.65 (s, 1H), 8.46 (d, J=2.4 Hz, 1H), 7.29 (d, J=9.2 Hz, 1H), 7.22 (dd, J=9.1, 3.1 Hz, 1H), 7.17 (d, J=3.0 Hz, 1H), 7.09 (dd, J=8.6, 2.4 Hz, 1H), 7.02 (d, J=8.6 Hz, 1H), 4.10 (t, J=6.3 Hz, 2H), 3.78 (s, 3H), 3.76 (s, 3H), 2.41 (s, 3H), 1.78 (dq, J=8.4, 6.4 Hz, 2H), 1.61-1.47 (m, 2H), 1.29 (s, 9H), 0.96 (t, J=7.4 Hz, 3H). ¹³C NMR (101 MHz, DMSO) δ 158.40, 153.06, 147.61, 145.34, 142.91, 138.87, 137.27, 126.74, 123.58, 120.44, 117.48, 116.27, 113.94, 113.88, 111.44, 68.08, 56.31, 55.84, 34.01, 31.30, 30.75, 18.65, 13.65, 8.71. ESI-TOF HRMS: m/z 467.2645 (C₂₆H₃₄N₄O₄+H⁺ requires 467.2653).

ff. Preparation of B7

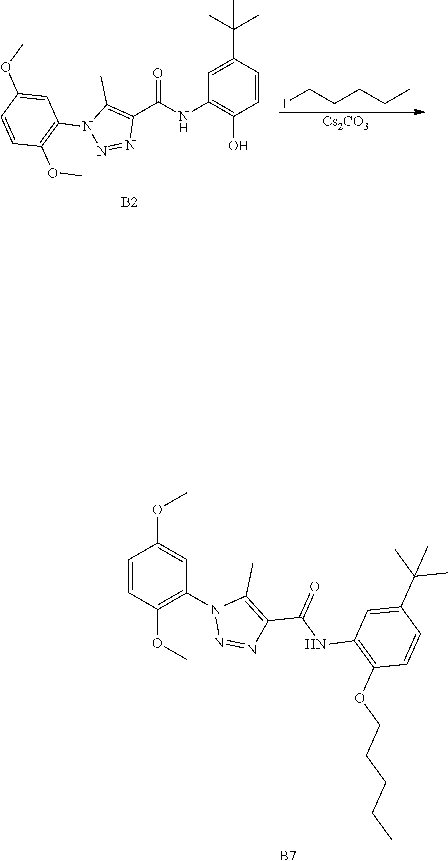

The preparation of compound B7 was prepared by reaction of N-(5-(tert-butyl)-2-hydroxyphenyl)-1-(2,5-dimethoxyphenyl)-5-methyl-1H-1,2,3-triazole-4-carboxamide and 1-iodopentane according to the procedure for B4 synthesis. 97.3 mg, 83% yield, white solid. ¹H NMR (500 MHz, DMSO-d6) δ 9.66 (s, 1H), 8.45 (d, J=2.4 Hz, 1H), 7.29 (d, J=9.2 Hz, 1H), 7.23 (dd, J=9.2, 3.1 Hz, 1H), 7.16 (d, J=3.1 Hz, 1H), 7.09 (dd, J=8.6, 2.4 Hz, 1H), 7.02 (d, J=8.6 Hz, 1H), 4.10 (t, J=6.3 Hz, 2H), 3.78 (s, 3H), 3.75 (s, 3H), 2.41 (s, 3H), 1.84-1.75 (m, 2H), 1.56-1.46 (m, 2H), 1.38 (dt, J=14.9, 7.3 Hz, 2H), 1.29 (s, 9H), 0.91 (t, J=7.3 Hz, 3H). ¹³C NMR (126 MHz, DMSO) δ 158.91, 153.55, 148.12, 145.87, 143.43, 139.36, 137.75, 127.26, 124.07, 120.97, 117.97, 116.77, 114.46, 114.38, 112.00, 68.90, 56.82, 56.34, 34.53, 31.81, 28.91, 28.12, 22.38, 14.39, 9.22. ESI-TOF HRMS: m/z 481.2825 (C₂₇H₃₆N₄O₄+H⁺ requires 481.2809).

gg. Preparation of B8

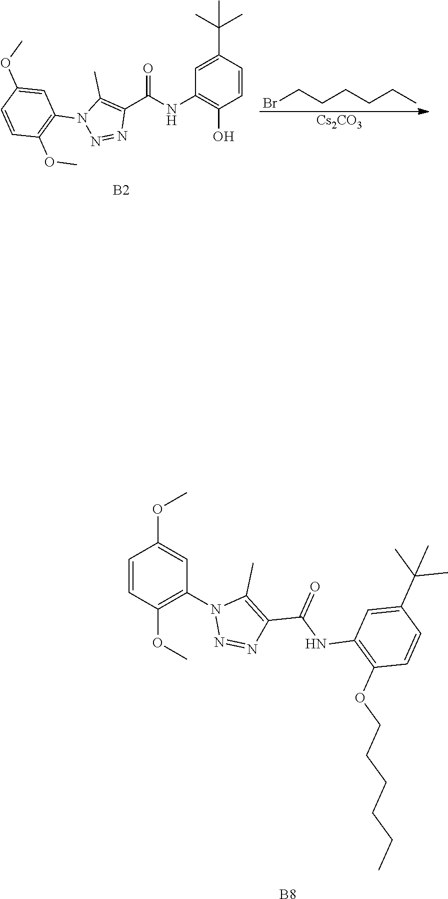

The preparation of compound B8 was prepared by reaction of N-(5-(tert-butyl)-2-hydroxyphenyl)-1-(2,5-dimethoxyphenyl)-5-methyl-1H-1,2,3-triazole-4-carboxamide and 1-bromohexane according to the procedure for B4 synthesis. 105.2 mg, 87% yield, colorless oil. ¹H NMR (500 MHz, DMSO-d6) δ 9.65 (s, 1H), 8.45 (d, J=2.4 Hz, 1H), 7.29 (d, J=9.2 Hz, 1H), 7.23 (dd, J=9.2, 3.0 Hz, 1H), 7.15 (d, J=3.0 Hz, 1H), 7.09 (dd, J=8.5, 2.4 Hz, 1H), 7.02 (d, J=8.6 Hz, 1H), 4.09 (t, J=6.3 Hz, 2H), 3.78 (s, 3H), 3.75 (s, 3H), 2.41 (s, 3H), 1.82-1.73 (m, 2H), 1.52 (tt, J=9.5, 6.1 Hz, 2H), 1.37-1.30 (m, 4H), 1.29 (s, 9H), 0.86 (t, J=7.0 Hz, 3H). ¹³C NMR (126 MHz, DMSO) δ 158.92, 153.55, 148.12, 145.88, 143.42, 139.35, 137.75, 127.25, 124.08, 120.98, 117.95, 116.80, 114.46, 114.39, 112.00, 68.92, 56.82, 56.34, 34.52, 31.82, 31.47, 29.18, 25.60, 22.53, 14.34, 9.22.

ESI-TOF HRMS: m/z 495.2966 (C₂₈H₃₈N₄O₄+H⁺ requires 495.2966).

hh. Preparation of B9

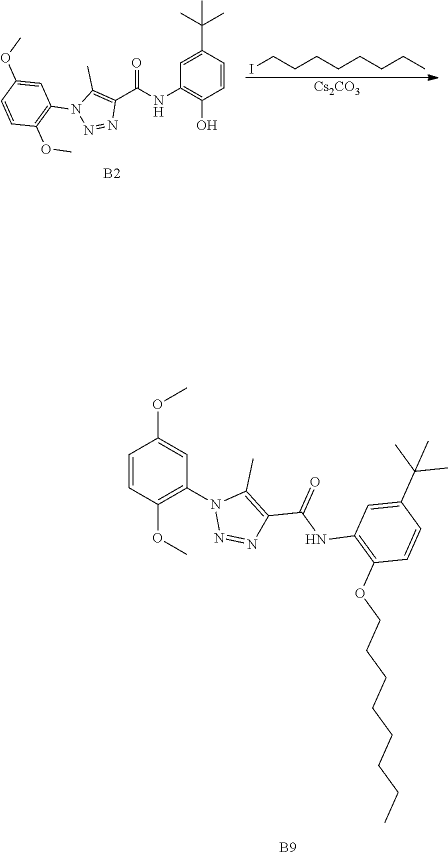

The preparation of compound B9 was prepared by reaction of N-(5-(tert-butyl)-2-hydroxyphenyl)-1-(2,5-dimethoxyphenyl)-5-methyl-1H-1,2,3-triazole-4-carboxamide and 1-iodooctane according to the procedure for B4 synthesis. 158.2 mg, 61% yield, colorless oil. ¹H NMR (400 MHz, DMSO-d6) δ 9.65 (s, 1H), 8.45 (d, J=2.4 Hz, 1H), 7.29 (d, J=9.2 Hz, 1H), 7.22 (dd, J=9.1, 3.0 Hz, 1H), 7.13 (d, J=3.0 Hz, 1H), 7.08 (dd, J=8.5, 2.4 Hz, 1H), 7.01 (d, J=8.6 Hz, 1H), 4.09 (t, J=6.3 Hz, 2H), 3.77 (s, 3H), 3.75 (s, 3H), 2.41 (s, 3H), 1.85-1.71 (m, 2H), 1.51 (p, J=7.1 Hz, 2H), 1.28 (s, 17H), 0.84-0.74 (m, 3H). ¹³C NMR (101 MHz, DMSO) δ 158.41, 153.05, 147.61, 145.33, 142.92, 138.82, 137.25, 126.77, 123.60, 120.43, 117.43, 116.26, 113.90, 113.88, 111.49, 68.41, 56.30, 55.82, 34.01, 31.29, 31.14, 28.69, 28.62, 25.42, 22.03, 13.84, 8.70. ESI-TOF HRMS: m/z 523.3279 (C₃₀H₄₂N₄O₄+H⁺ requires 523.3279).

ii. Preparation of B10

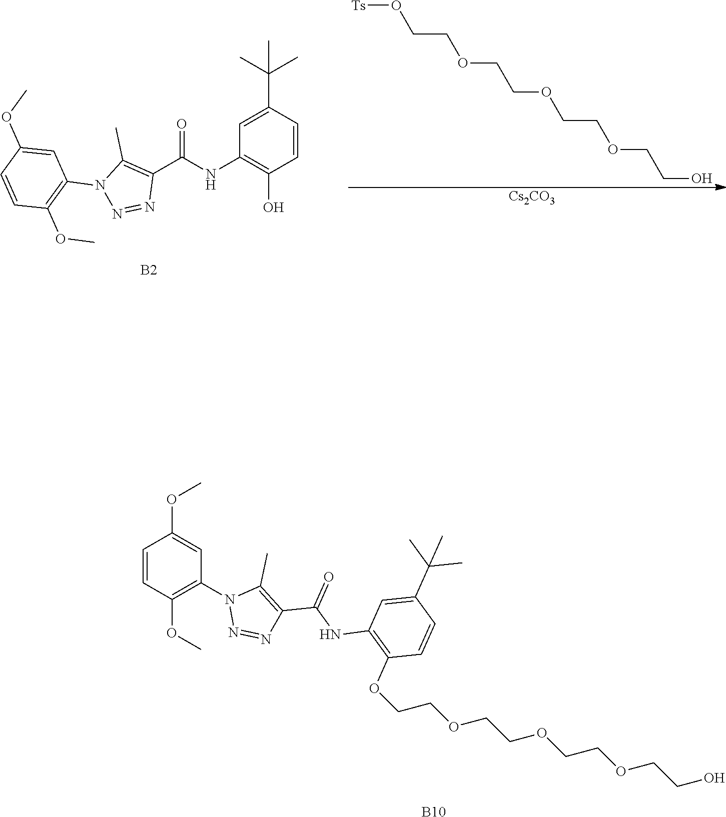

The preparation of compound B10 was prepared by reaction of N-(5-(tert-butyl)-2-hydroxyphenyl)-1-(2,5-dimethoxyphenyl)-5-methyl-1H-1,2,3-triazole-4-carboxamide and 2-(2-(2-(2-hydroxyethoxy)ethoxy)ethoxy)ethyl 4-methylbenzenesulfonate according to the procedure for B4 synthesis. 198.5 mg, 68% yield, colorless oil. ¹H NMR (400 MHz, DMSO-d6) δ 9.64 (s, 1H), 8.46 (d, J=2.3 Hz, 1H), 7.29 (d, J=9.2 Hz, 1H), 7.22 (dd, J=9.1, 3.0 Hz, 1H), 7.15 (d, J=3.0 Hz, 1H), 7.10 (dd, J=8.6, 2.3 Hz, 1H), 7.05 (d, J=8.6 Hz, 1H), 4.54 (s, 1H), 4.29-4.18 (m, 2H), 3.84-3.80 (m, 2H), 3.78 (s, 3H), 3.76 (s, 3H), 3.66 (dd, J=5.8, 3.7 Hz, 2H), 3.55 (dd, J=5.8, 3.8 Hz, 2H), 3.51-3.41 (m, 6H), 3.36 (dd, J=6.3, 3.7 Hz, 6H), 2.41 (s, 3H), 1.29 (s, 9H). ¹³C NMR (101 MHz, DMSO) δ 158.51, 153.06, 147.63, 145.31, 143.34, 138.89, 137.25, 127.04, 123.60, 120.52, 117.43, 116.55, 113.93, 113.88, 112.24, 72.27, 70.15, 69.79, 69.74, 69.66, 68.93, 68.76, 60.14, 56.30, 55.82, 34.03, 31.28, 8.71. ESI-TOF HRMS: m/z 587.3087 (C₃₀H₄₂N₄O₈+H⁺ requires 587.3075).

jj. Preparation of B11

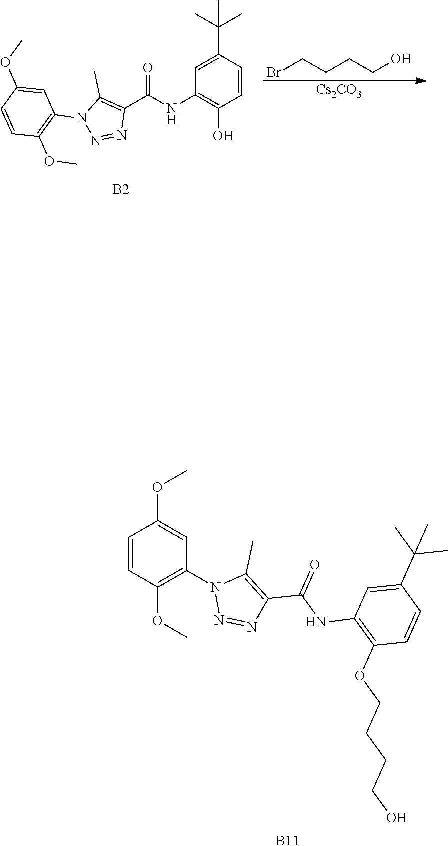

The preparation of compound B11 was prepared by reaction of N-(5-(tert-butyl)-2-hydroxyphenyl)-1-(2,5-dimethoxyphenyl)-5-methyl-1H-1,2,3-triazole-4-carboxamide and 4-bromobutan-1-ol according to the procedure for B4 synthesis. 329.5 mg, 30% yield, white solid. ¹H NMR (500 MHz, CDCl₃) δ 9.80 (s, 1H), 8.64 (d, J=2.3 Hz, 1H), 7.08 (ddd, J=8.5, 5.1, 2.7 Hz, 2H), 7.02 (d, J=9.2 Hz, 1H), 6.96 (d, J=3.0 Hz, 1H), 6.86 (d, J=8.6 Hz, 1H), 4.13 (t, J=5.9 Hz, 2H), 3.81 (d, J=3.4 Hz, 5H), 3.75 (s, 3H), 2.54 (s, 3H), 2.07-1.96 (m, 2H), 1.90 (dt, J=8.3, 6.3 Hz, 2H), 1.36 (s, 9H). ¹³C NMR (126 MHz, CDCl₃) δ 159.22, 153.68, 148.00, 145.58, 144.05, 139.25, 138.42, 127.54, 124.37, 120.17, 117.55, 116.95, 113.74, 113.30, 110.65, 68.83, 62.35, 56.34, 56.02, 34.48, 31.59, 29.72, 25.51, 9.30. ESI-TOF HRMS: m/z 483.2599 (C₂₆H₃₄N₄O₅+H^{|} requires 483.2602).

kk. Preparation of B12

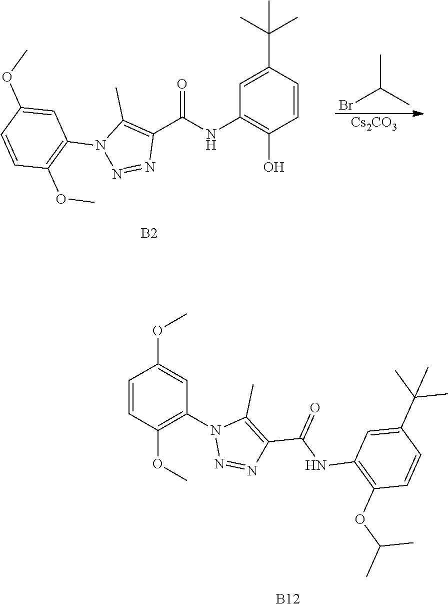

The preparation of compound B12 was prepared by reaction of N-(5-(tert-butyl)-2-hydroxyphenyl)-1-(2,5-dimethoxyphenyl)-5-methyl-1H-1,2,3-triazole-4-carboxamide and 2-bromopropane according to the procedure for B4 synthesis. 94.1 mg, 86% yield, white solid. ¹H NMR (500 MHz, DMSO-d6) δ 9.64 (s, 1H), 8.48 (d, J=2.3 Hz, 1H), 7.29 (d, J=9.2 Hz, 1H), 7.23 (dd, J=9.2, 3.1 Hz, 1H), 7.17 (d, J=3.0 Hz, 1H), 7.10 (dd, J=8.6, 2.4 Hz, 1H), 7.05 (d, J=8.7 Hz, 1H), 4.65 (p, J=6.1 Hz, 1H), 3.78 (s, 3H), 3.76 (s, 3H), 2.41 (s, 3H), 1.35 (d, J=6.0 Hz, 6H), 1.29 (s, 9H). ¹³C NMR (126 MHz, DMSO) δ 158.84, 153.55, 148.10, 144.54, 143.67, 139.39, 137.79, 128.26, 124.07, 120.97, 117.97, 116.84, 114.45, 114.39, 114.08, 71.94, 56.82, 56.34, 34.55, 31.80, 22.50, 9.24. ESI-TOF HRMS: m/z 453.2501 (C₂₅H₃₂N₄O₄+H⁺ requires 453.2496).

ll. Preparation of B13

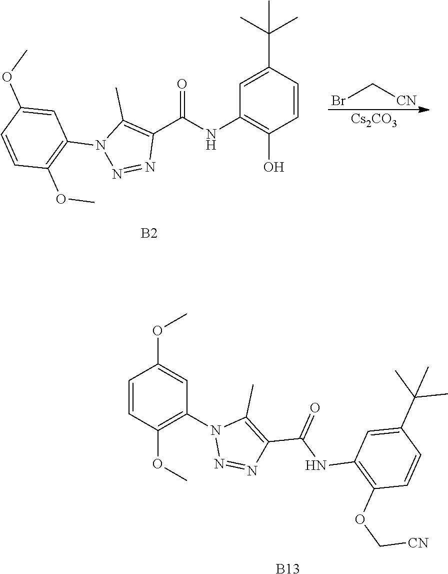

The preparation of compound B13 was prepared by reaction of N-(5-(tert-butyl)-2-hydroxyphenyl)-1-(2,5-dimethoxyphenyl)-5-methyl-1H-1,2,3-triazole-4-carboxamide and 2-bromoacetonitrile according to the procedure for B4 synthesis. 81.9 mg, 74% yield, white solid. ¹H NMR (500 MHz, DMSO-d6) δ 9.60 (s, 1H), 8.31 (d, J=2.1 Hz, 1H), 7.29 (d, J=9.2 Hz, 1H), 7.25-7.18 (m, 3H), 5.31 (s, 2H), 3.78 (s, 3H), 3.76 (s, 3H), 2.40 (s, 3H), 1.30 (s, 9H). ¹³C NMR (126 MHz, DMSO) δ 159.21, 153.55, 148.14, 145.58, 144.81, 139.53, 137.60, 127.33, 124.06, 121.72, 119.03, 117.95, 117.09, 114.50, 114.40, 112.90, 56.83, 56.35, 54.87, 34.66, 31.70, 9.24. ESI-TOF HRMS: m/z 450.2132 (C₂₄H₂₇N₅O₄+H⁺ requires 450.2136).

mm. Preparation of B14

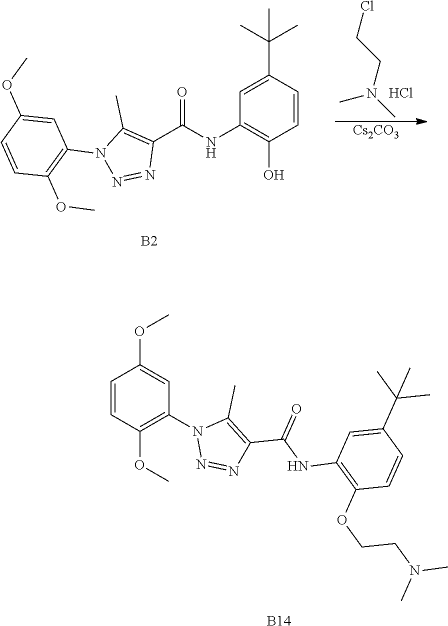

To a solution of N-(5-(tert-butyl)-2-hydroxyphenyl)-1-(2,5-dimethoxyphenvl)-5-methyl-1H-1,2,3-triazole-4-carboxamide (100 mg, 0.244 mmol) in DMF 5 mL was added Cs₂CO₃ (175 mg, 0.536 mmol) and 2-chloro-N,N-dimethylethan-1-amine hydrochloride (42.1 mg, 0.292 mmol) at room temperature. The suspension was stirred at 60° C. for overnight. The reaction mixture was then diluted with water (50 mL) and extracted with EA (50 mL×2). The combine EA layers were washed with water, dried with anhydrous Na₂SO₄, and concentrated. The residue was purified by silica gel chromatography (0% to 100% EA in hexane) to give product as a white solid (67.1 mg, 57% yield). ¹H NMR (500 MHz, DMSO-d6) δ 9.92 (s, 1H), 8.42 (d, J=2.3 Hz, 1H), 7.29 (d, J=9.2 Hz, 1H), 7.22 (dd, J=9.2, 3.1 Hz, 1H), 7.16 (d, J=3.0 Hz, 1H), 7.11 (dd, J=8.6, 2.4 Hz, 1H), 7.07 (d, J=8.6 Hz, 1H), 4.20 (t, J=5.5 Hz, 2H), 3.78 (s, 3H), 3.75 (s, 3H), 2.68 (t, J=5.5 Hz, 2H), 2.40 (s, 3H), 2.27 (s, 6H), 1.29 (s, 9H). ¹³C NMR (126 MHz, CDCl₃) δ 159.47, 153.68, 148.03, 145.36, 144.50, 139.15, 138.46, 127.78, 124.44, 120.33, 117.48, 117.45, 113.75, 113.30, 111.35, 67.29, 58.01, 56.33, 56.01, 45.77, 34.50, 31.56, 9.30. ESI-TOF HRMS: m/z 482.2776 (C₂₆H₃₅N₅O₄+H⁺ requires 482.2762).

nn. Preparation of B15

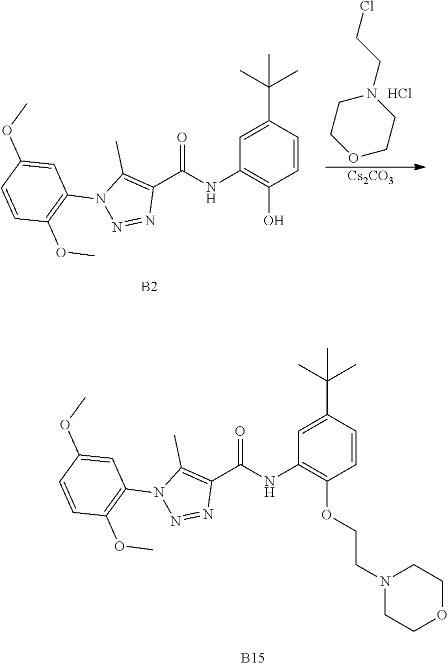

The preparation of compound B15 was prepared by reaction of N-(5-(tert-butyl)-2-hydroxyphenyl)-1-(2,5-dimethoxyphenyl)-5-methyl-1H-1,2,3-triazole-4-carboxamide and 4-(2-chloroethyl)morpholine hydrochloride according to the procedure for B14 synthesis. 88.5 mg, 69% yield, white solid. ¹H NMR (500 MHz, Chloroform-d) δ 9.75 (s, 1H), 8.62 (d, J=2.4 Hz, 1H), 7.08 (ddd, J=8.6, 4.7, 2.7 Hz, 2H), 7.03 (d, J=9.1 Hz, 1H), 6.95 (d, J=3.1 Hz, 1H), 6.87 (d, J=8.6 Hz, 1H), 4.30 (s, 2H), 3.82 (s, 3H), 3.76 (s, 8H), 3.01 (s, 2H), 2.90-2.68 (m, 4H), 2.53 (s, 3H), 1.35 (s, 9H). ^{13C} NMR (126 MHz, CDCl3) δ 159.39, 153.69, 148.03, 145.25, 144.61, 139.18, 138.34, 127.59, 124.40, 120.30, 117.49, 117.37, 113.75, 113.32, 111.03, 66.82, 66.52, 57.62, 56.34, 56.02, 53.96, 34.51, 31.56, 9.28. ESI-TOF HRMS: m/z 524.2872 (C₂₈H₃₇N₅O₅+H⁺ requires 524.2867).

oo. Preparation of B16

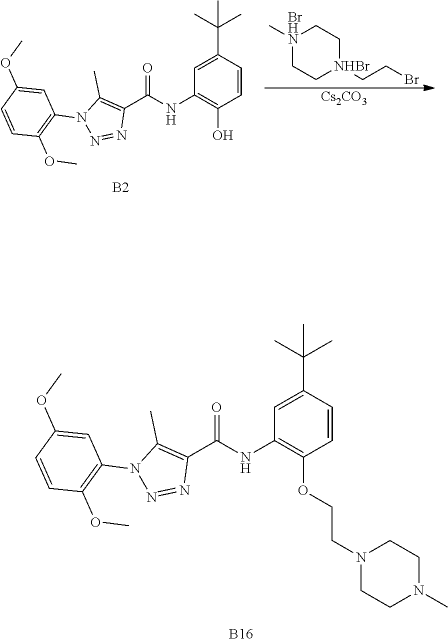

To a solution of N-(5-(tert-butyl)-2-hydroxyphenyl)-1-(2,5-dimethoxyphenvl)-5-methyl-1H-1,2,3-triazole-4-carboxamide (100 mg, 0.244 mmol) in DMF 5 mL at rt was added Cs₂CO₃ (254 mg, 0.780 mmol) and 1-(2-bromoethyl)-4-methylpiperazine dihydrobromide (108 mg, 0.292 mmol). The suspension was stirred at 60° C. for overnight. The reaction mixture was then diluted with water (50 mL) and extracted with EtOAc (50 mL×2). The combine EtOAc layers were washed with water, dried with anhydrous Na₂SO₄, and concentrated. The residue was purified by silica gel chromatography (0% to 30% methanol in dichloromethane) to give product (51.9 mg, 40% yield) as a white solid. ¹H NMR (500 MHz, Chloroform-d) δ 9.78 (s, 1H), 8.61 (d, J=2.3 Hz, 1H), 7.08 (ddd, J=8.5, 7.3, 2.7 Hz, 2H), 7.03 (d, J=9.1 Hz, 1H), 6.94 (d, J=3.0 Hz, 1H), 6.88 (d, J=8.6 Hz, 1H), 4.21 (t, J=5.6 Hz, 2H), 3.82 (s, 3H), 3.76 (s, 3H), 2.91 (t, J=5.6 Hz, 2H), 2.88-2.62 (m, 8H), 2.53 (s, 3H), 2.41 (s, 3H), 1.35 (s, 9H). ¹³C NMR (126 MHz, CDCl3) δ 159.44, 153.69, 147.99, 145.36, 144.56, 139.28, 138.39, 127.92, 124.41, 120.37, 117.47, 117.36, 113.86, 113.28, 111.64, 66.73, 56.73, 56.30, 56.02, 54.47, 52.41, 45.15, 34.51, 31.56, 9.30. ESI-TOF HRMS: m/z 537.3182 (C₂₉H₄₀N₆O₄+H⁺ requires 537.3184).

pp. Preparation of B17

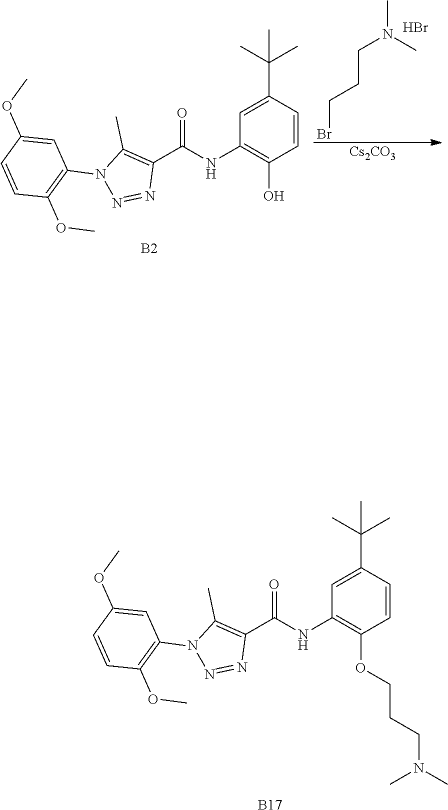

The preparation of compound B17 was prepared by reaction of N-(5-(tert-butyl)-2-hydroxyphenyl)-1-(2,5-dimethoxyphenyl)-5-methyl-1H-1,2,3-triazole-4-carboxamide and 3-bromo-N,N-dimethylpropan-1-amine hydrobromide according to the procedure for B14 synthesis. 88.9 mg, 73% yield, white solid. ¹H NMR (500 MHz, Chloroform-d) δ 9.78 (s, 1H), 8.62 (d, J=2.4 Hz, 1H), 7.11-7.05 (m, 2H), 7.03 (d, J=9.1 Hz, 1H), 6.95 (d, J=3.0 Hz, 1H), 6.87 (d, J=8.6 Hz, 1H), 4.15 (t, J=6.1 Hz, 2H), 3.82 (s, 3H), 3.76 (s, 3H), 2.89-2.74 (m, 2H), 2.53 (s, 3H), 2.44 (s, 6H), 2.32-2.12 (m, 2H), 1.35 (s, 9H). ¹³C NMR (126 MHz, CDCl3) δ 159.27, 153.69, 148.04, 145.35, 144.25, 139.15, 138.45, 127.50, 124.43, 120.19, 117.37, 116.98, 113.88, 113.30, 110.73, 66.69, 56.32, 56.23, 56.01, 45.02, 34.49, 31.57, 26.95, 9.26. ESI-TOF HRMS: m/z 496.2916 (C₂₇H₃₇N₅O₄+H⁺ requires 496.2918).

qq. Preparation of B18

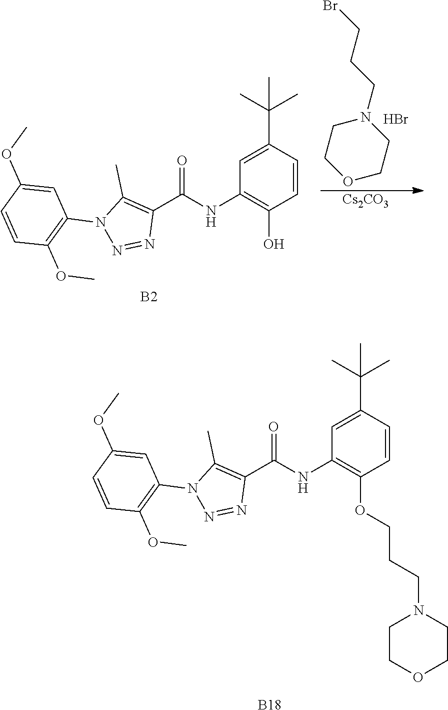

The preparation of compound B18 was prepared by reaction of N-(5-(tert-butyl)-2-hydroxyphenyl)-1-(2,5-dimethoxyphenyl)-5-methyl-1H-1,2,3-triazole-4-carboxamide and 4-(3-bromopropyl)morpholine hydrobromide according to the procedure for B14 synthesis. 92.9 mg, 71% yield, white solid. ¹H NMR (500 MHz, Chloroform-d) δ 9.78 (s, 1H), 8.63 (d, J=2.4 Hz, 1H), 7.08 (ddd, J=8.5, 7.7, 2.7 Hz, 2H), 7.03 (d, J=9.1 Hz, 1H), 6.95 (d, J=3.1 Hz, 1H), 6.87 (d, J=8.6 Hz, 1H), 4.16 (t, J=6.1 Hz, 2H), 3.82 (s, 3H), 3.80-3.63 (m, 7H), 2.53 (s, 9H), 2.11 (s, 2H), 1.35 (s, 9H). ¹³C NMR (126 MHz, CDCl3) δ 159.31, 153.69, 148.01, 145.39, 144.25, 139.14, 138.40, 127.54, 124.45, 120.16, 117.38, 117.04, 113.85, 113.30, 110.75, 66.71, 56.33, 56.01, 55.46, 53.53, 34.49, 31.57, 26.14, 9.27.

ESI-TOF HRMS: m/z 538.3035 (C₂₉H₃₉N₅O₅+H⁺ requires 538.3024).

rr. Preparation of B20

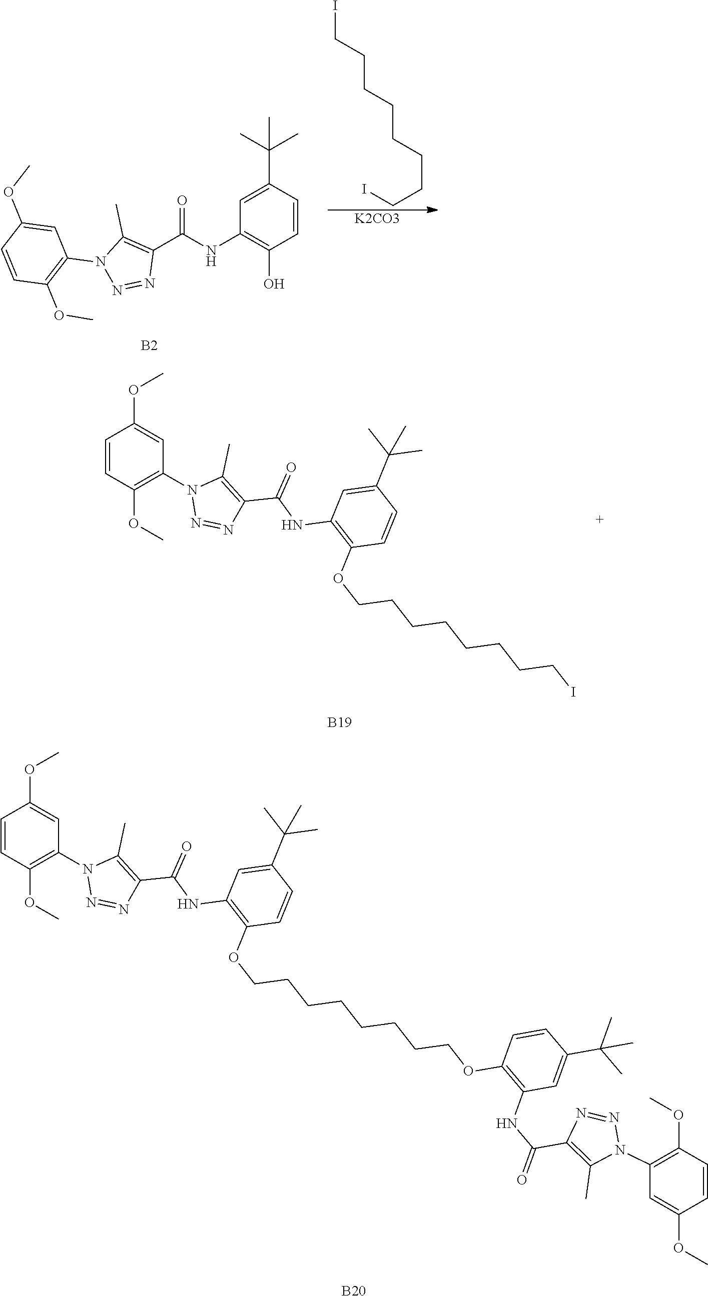

To a solution of N-(5-(tert-butyl)-2-hydroxyphenyl)-1-(2,5-dimethoxyphenyl)-5-methyl-1H-1,2,3-triazole-4-carboxamide (1.642 g, 4 mmol) in DMF 15 mL was added K₂CO₃ (1.106 g, 8.00 mmol) at room temperature. The reaction mixture was stirred at room temperature for 15 mins and then to the solution was added 1,8-diiodooctane (1.464 g, 4.00 mmol). The suspension was stirred at room temperature for overnight. The solvent was diluted with water (100 mL) and extracted with EA (200 mL). The organic phase washed by NaHCO₃ saturated solution (100 mL), water (100 mL), and brine (100 mL). The organic phase dried by MgSO₄, filtered, and evaporated in vacuum. The residue was purified to providing N-(5-(tert-butyl)-2-((8-iodooctyl)oxy)phenyl)-1-(2,5-dimethoxyphenyl)-5-methyl-1H-1,2,3-triazole-4-carboxamide (1.39 g, 54% yield) as a light brown solid. ¹H NMR (400 MHz, DMSO-d6) δ 9.66 (s, 1H), 8.47 (d, J=2.4 Hz, 1H), 7.28 (d, J=9.2 Hz, 1H), 7.22 (dd, J=9.1, 3.1 Hz, 1H), 7.14 (d, J=3.0 Hz, 1H), 7.08 (dd, J=8.6, 2.4 Hz, 1H), 7.00 (d, J=8.7 Hz, 1H), 4.08 (t, J=6.2 Hz, 2H), 3.78 (s, 3H), 3.75 (s, 3H), 3.20 (t, J=6.9 Hz, 2H), 2.41 (s, 3H), 1.78 (dd, J=8.5, 6.2 Hz, 2H), 1.71 (t, J=6.8 Hz, 2H), 1.59-1.46 (m, 2H), 1.40-1.30 (m, 6H), 1.28 (s, 9H). ¹³C NMR (101 MHz, DMSO) δ 158.39, 153.06, 147.60, 145.28, 142.93, 138.83, 137.27, 126.79, 123.58, 120.39, 117.44, 116.19, 113.89, 111.45, 68.38, 56.33, 55.84, 34.01, 32.91, 31.30, 29.71, 28.66, 28.54, 27.79, 25.34, 8.81, 8.73. ESI-TOF HRMS: m/z 649.2256 (C₃₀H₄₁IN₄O₄+H⁺ requires 649.2245).

N,N-((octane-1,8-diylbis(oxy))bis(5-(tert-butyl)-2,1-phenylene))bis(1-(2,5-dimethoxyphenyl)-5-methyl-1H-1,2,3-triazole-4-carboxamide) as white solid, 432.8 mg, 23 % yield. ¹H NMR (400 MHz, DMSO) δ 9.63 (s, 1H), 8.46 (d, J=2.4 Hz, 1H), 7.25 (d, J=9.2 Hz, 1H), 7.18 (dd, J=9.1, 3.0 Hz, 1H), 7.13 (d, J=3.0 Hz, 1H), 7.01 (dd, J=8.6, 2.4 Hz, 1H), 6.90 (d, J=8.7 Hz, 1H), 4.10-3.97 (m, 2H), 3.74 (s, 3H), 3.72 (s, 3H), 2.40 (s, 3H), 1.76 (p, J=6.5 Hz, 2H), 1.51 (q, J=7.0, 6.4 Hz, 2H), 1.43-1.33 (m, 2H), 1.26 (s, 9H). ¹³C NMR (101 MHz, DMSO) δ 158.37, 153.05, 147.58, 145.25, 142.82, 138.79, 137.28, 126.76, 123.61, 120.28, 117.33, 116.20, 113.90, 113.80, 111.25, 68.33, 56.24, 55.75, 33.96, 31.26, 28.70, 28.63, 25.22, 8.69. ESI-TOF HRMS: m/z 931.5073 (C₅₂H₆₆N₈O₈+H⁺ requires 931.5076).

ss. Preparation of B21

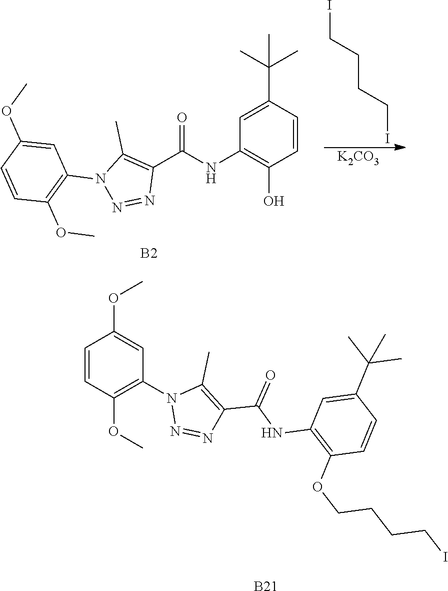

To a solution of N-(5-(tert-butyl)-2-hydroxyphenyl)-1-(2,5-dimethoxyphenvl)-5-methyl-1H-1,2,3-triazole-4-carboxamide (0.92 g, 2.241 mmol) in DMF 15 mL was added K₂CO₃ (3.10 g, 22.41 mmol) at room temperature. The reaction mixture was stirred at room temperature for 15 mins and then to the solution was added 1,4-diiodobutane (2.96 ml, 22.41 mmol). The suspension was stirred for overnight. The organic phase washed by NaHCO₃ saturated solution (100 mL), water (100 mL), and brine (100 mL), The organic phase dried by MgSO₄, filtered, and evaporated in vacuum. The residue was purified to providing N-(5-(tert-butyl)-2-(4-iodobutoxy)phenyl)-1-(2,5-dimethoxyphenyl)-5-methyl-1H-1,2,3-triazole-4-carboxamide (445 mg, 36% yield) as a light red solid. ¹H NMR (400 MHz, DMSO-d6) δ 9.63 (s, 1H), 8.41 (d, J=2.4 Hz, 1H), 7.29 (d, J=9.2 Hz, 1H), 7.22 (dd, J=9.1, 3.1 Hz, 1H), 7.16 (d, J=3.0 Hz, 1H), 7.11 (dd, J=8.6, 2.4 Hz, 1H), 7.03 (d, J=8.6 Hz, 1H), 4.14 (t, J=6.3 Hz, 2H), 3.78 (s, 3H), 3.76 (s, 3H), 3.37 (t, J=6.8 Hz, 2H), 2.41 (s, 3H), 2.02 (dq, J=8.5, 6.8 Hz, 2H), 1.89 (dq, J=9.4, 6.3 Hz, 2H), 1.29 (s, 9H). 13C NMR (101 MHz, DMSO) δ 158.45, 153.06, 147.62, 145.43, 143.04, 138.85, 137.26, 126.66, 123.59, 120.58, 117.46, 116.63, 113.96, 113.90, 111.58, 67.34, 56.33, 55.85, 34.02, 31.30, 29.73, 29.56, 8.73, 8.23.

ESI-TOF HRMS: m/z 593.1624 (C26H33IN4O4+H^{|} requires 593.1619).

tt. Preparation of B22

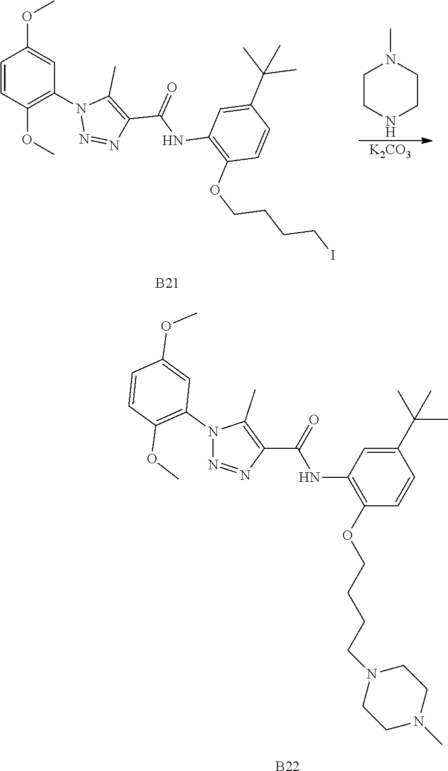

To a solution of N-(5-(tert-butyl)-2-(4-iodobutoxy)phenyl)-1-(2,5-dimethoxyphenyl)-5-methyl-1H-1,2,3-triazole-4-carboxamide (0.118 g, 0.2 mmol) in DMF 5 mL at rt was added K₂CO₃ (0.041 g, 0.300 mmol) and 1-methylpiperazine (0.030 g, 0.300 mmol). The suspension was stirred overnight. The solvent was diluted with water (100 mL) and extracted with EA (200 mL). The organic solvent washed by NaHCO₃ saturated solution (100 mL), water (100 mL), and brine (100 mL). The organic phase dried by MgSO₄, filtered, and evaporated in vacuum. The residue was purified to providing N-(5-(tert-butyl)-2-(4-(4-methylpiperazin-1-yl)butoxy)phenyl)-1-(2,5-dimethoxyphenyl)-5-methyl-1H-1,2,3-triazole-4-carboxamide (75.9 mg, 67% yield) as alight yellow solid. ¹H NMR (400 MHz, DMSO-d6) δ 9.65 (s, 1H), 8.46 (d, J=2.3 Hz, 1H), 7.28 (d, J=9.2 Hz, 1H), 7.22 (dd, J=9.2, 2.9 Hz, 1H), 7.15 (d, J=2.9 Hz, 1H), 7.08 (dd, J=8.6, 2.4 Hz, 1H), 7.00 (d, J=8.6 Hz, 1H), 4.10 (t, J=5.9 Hz, 2H), 3.78 (s, 3H), 3.76 (s, 3H), 2.70-2.17 (m, 16H), 1.87-1.63 (m, 4H), 1.28 (s, 9H). ¹³C NMR (101 MHz, DMSO) δ 158.37, 153.07, 147.60, 145.30, 142.92, 138.86, 137.28, 126.70, 123.59, 120.41, 117.40, 116.30, 113.93, 113.87, 111.35, 68.19, 56.86, 56.30, 55.81, 54.85, 53.60, 51.51, 33.99, 31.28, 26.58, 22.41, 8.71. ESI-TOF HRMS: m/z 565.3500 (C₃₁H₄₄N₆O₄+H⁺ requires 565.3497).

uu. Preparation of B23

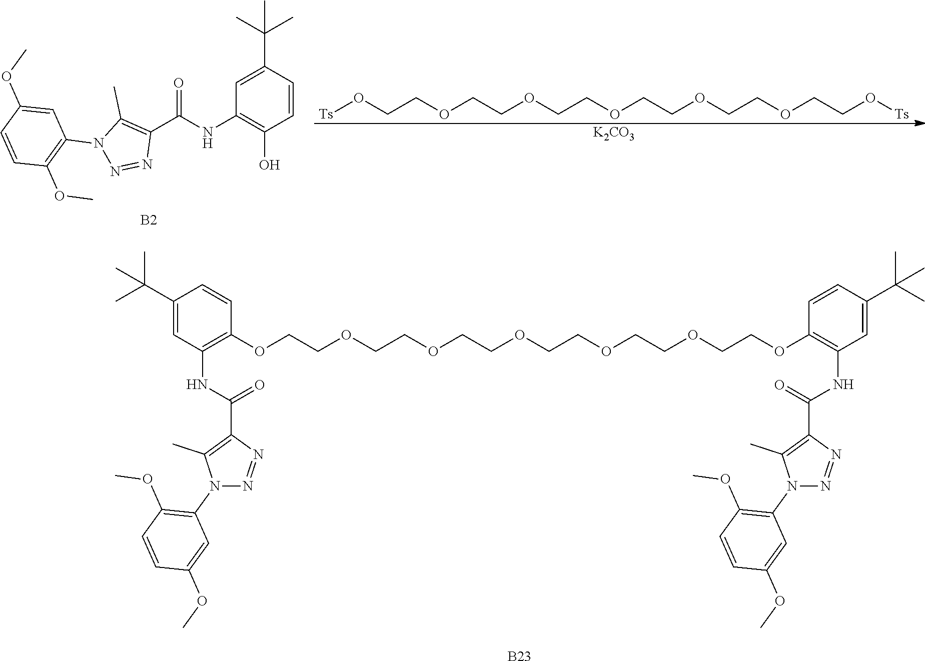

To a solution of N-(5-(tert-butyl)-2-hydroxyphenyl)-1-(2,5-dimethoxyphenvl)-5-methyl-1H-1,2,3-triazole-4-carboxamide (0.205 g, 0.5 mmol) in DMF 5 mL was added K₂CO₃ (0.083 g, 0.600 mmol) and 3,6,9,12,15-pentaoxaheptadecane-1,17-diyl bis(4-methylbenzenesulfonate) (0.140 g, 0.238 mmol). The suspension was stirred at 60° C. for overnight. Then the reaction mixture was diluted with water (50 mL) and extracted with EA (100 mL). The organic solvent washed by NaHCO₃ saturated solution (50 mL), water (50 mL), and brine (50 mL). The organic phase was dried over MgSO₄, filtered, and concentrated. The residue was purified to providing N,N′-((3,6,9,12,15-pentaoxaheptadecane-1,17-diylbis(oxy))bis(3-(tert-butyl)-6,1-phenylene))bis(1-(2,5-dimethoxyphenyl)-5-methyl-1H-1,2,3-triazole-4-carboxamide) (175.8 mg, 69% yield) as a white solid. ¹H NMR (400 MHz, DMSO-d6) δ 9.64 (s, 2H), 8.47 (d, 0.1=2.3 Hz, 2H), 7.26 (d, J=9.2 Hz, 2H), 7.20 (dd, J=9.1, 3.0 Hz, 2H), 7.14 (d, J=3.0 Hz, 2H), 7.08 (dd, J=8.6, 2.4 Hz, 2H), 7.02 (d, J=8.6 Hz, 2H), 4.25-4.17 (m, 4H), 3.82-3.77 (m, 6H), 3.76 (s, 6H), 3.74 (s, 5H), 3.62 (dd, J=5.8, 3.8 Hz, 4H), 3.53-3.49 (m, 4H), 3.44-3.37 (m, 8H), 2.40 (s, 6H), 1.28 (s, 18H). ¹³C NMR (101 MHz, DMSO) δ 158.49, 153.05, 147.61, 145.27, 143.32, 138.86, 137.25, 127.05, 123.60, 120.47, 117.37, 116.50, 113.94, 113.83, 112.18, 70.12, 69.75, 69.66, 69.61, 68.91, 68.74, 56.27, 55.79, 34.02, 31.26, 8.70. ESI-TOF HRMS: m/z 1067.5458 (C₅₆H₇₄N₈O₁₃+H⁺ requires 1067.5448).

vv. Preparation of B24

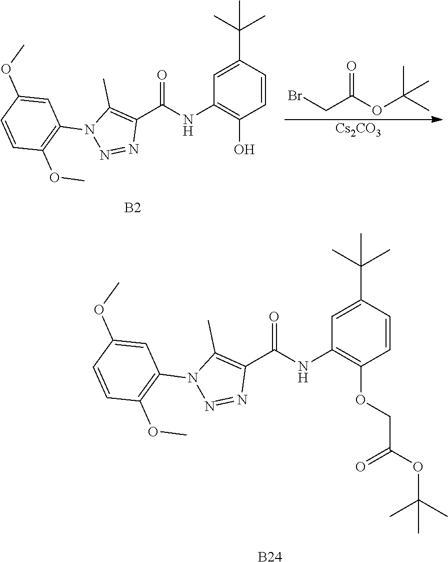

Tert-butyl 2-bromoacetate (1.108 ml, 7.50 mmol) was added to a suspension solution of N-(5-(tert-butyl)-2-hydroxyphenyl)-1-(2,5-dimethoxyphenyl)-5-methyl-1H-1,2,3-triazole-4-carboxamide (2.8 g, 6.82 mmol) and Cs₂CO₃ (2.67 g, 8.19 mmol) in DMF (5 mL). Then the reaction mixture was stirred at 60° C. for 3 h. The reaction mixture was cool down and then diluted with water (50 mL) and extracted with EA (50 mL×2). The combine EA layers were washed with water (50 mL), dried with anhydrous Na₂SO₄, and concentrated. The residue was purified by silica gel chromatography (0% to 100% EA in hexane) to give product as a white solid (3.08 g, 96% yield). ¹H NMR (500 MHz, DMSO-d6) δ 9.68 (s, 1H), 8.46 (d, J=2.4 Hz, 1H), 7.29 (d, J=9.2 Hz, 1H), 7.23 (dd, J=9.1, 3.1 Hz, 1H), 7.16 (d, J=3.0 Hz, 1H), 7.10 (dd, J=8.6, 2.4 Hz, 1H), 6.97 (d, J=8.7 Hz, 1H), 4.82 (s, 2H), 3.78 (s, 3H), 3.76 (s, 3H), 2.41 (s, 3H), 1.44 (s, 9H), 1.29 (s, 9H). ¹³C NMR (126 MHz, DMSO) δ 167.22, 157.96, 152.48, 147.07, 144.14, 143.23, 138.40, 136.59, 126.38, 123.00, 119.93, 116.88, 116.28, 113.40, 113.32, 111.42, 81.09, 65.52, 55.76, 55.28, 33.51, 30.70, 27.10, 8.17.

ESI-TOF HRMS: m/z 525.2710 (C₂₈H₃₆N₄O₆+H⁺ requires 525.2708).

ww. Preparation of B25

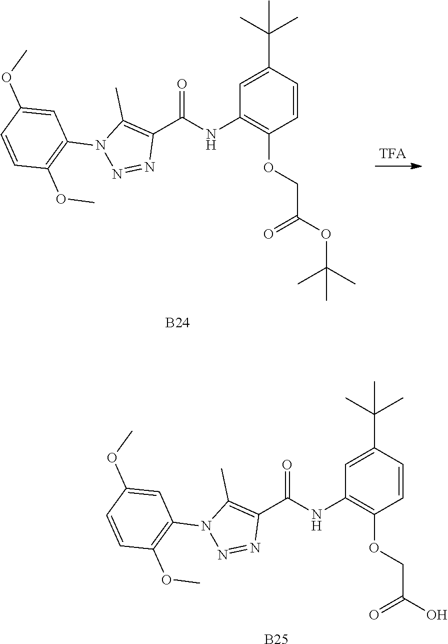

A stirred mixture containing tert-butyl 2-(4-(tert-butyl)-2-(1-(2,5-dimethoxyphenyl)-5-methyl-1H-1,2,3-triazole-4-carboxamido)phenoxy)acetate (3 g, 5.72 mmol) in 20 ml dry DCM, 10 mL of TFA was added and stirred at room temperature for 20 h. Then the mixture was concentrated, and the residue mixture was added EA (100 mL) and washed by water (100 mL). The organic phase was concentrated under the reduced pressure. The residue was purified by silica gel chromatography to give product (2.11 g, 79%) as a white solid. ¹H NMR (500 MHz, DMSO-d6) δ 9.74 (s, 1H), 8.43 (d, J=2.4 Hz, 1H), 7.29 (d, J=9.2 Hz, 1H), 7.23 (dd, J=9.1, 3.1 Hz, 1H), 7.16 (d, J=3.0 Hz, 1H), 7.10 (dd, J=8.7, 2.4 Hz, 1H), 6.99 (d, J=8.7 Hz, 1H), 4.86 (s, 2H), 3.78 (s, 3H), 3.76 (s, 3H), 2.41 (s, 3H), 1.29 (s, 9H). ¹³C NMR (126 MHz, DMSO) δ 169.71, 157.98, 152.47, 147.07, 144.41, 143.20, 138.39, 136.63, 126.46, 123.01, 120.04, 116.86, 116.49, 113.41, 113.31, 111.74, 65.23, 55.75, 55.27, 33.49, 30.69, 8.18. ESI-TOF HRMS: m/z 469.2087 (C₂₄H₂₈N₄O₆+H⁺ requires 469.2082).

xx. Preparation of B26

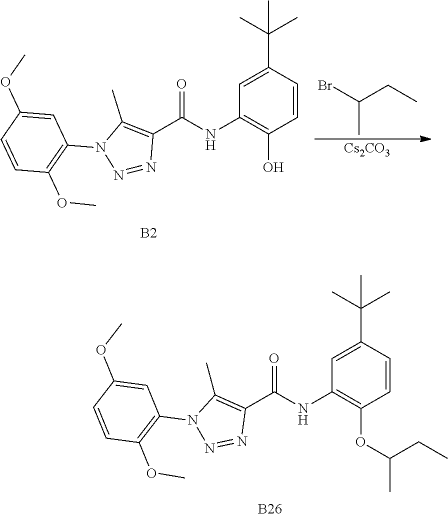

The preparation of compound B26 was prepared by reaction of N-(5-(tert-butyl)-2-hydroxyphenyl)-1-(2,5-dimethoxyphenyl)-5-methyl-1H-1,2,3-triazole-4-carboxamide and 2-bromobutane according to the procedure for B4 synthesis. 88.7 mg, 79% yield, white solid. ¹H NMR (500 MHz, Chloroform-d) δ 9.86 (s, 1H), 8.66 (d, J=2.4 Hz, 1H), 7.08 (dd, J=9.1, 3.0 Hz, 1H), 7.06 (dd, J=8.5, 2.4 Hz, 1H), 7.03 (d, J=9.1 Hz, 1H), 6.96 (d, J=3.0 Hz, 1H), 6.86 (d, J=8.6 Hz, 1H), 4.37 (h, J=6.0 Hz, 1H), 3.81 (s, 3H), 3.76 (s, 3H), 2.54 (s, 3H), 1.93-1.82 (m, 1H), 1.79-1.68 (m, 1H), 1.38 (d, J=6.1 Hz, 3H), 1.36 (s, 9H). 1.05 (t, J=7.4 Hz, 3H). ¹³C NMR (126 MHz, CDCl3) δ 159.40, 153.68, 148.01, 144.69, 143.83, 139.06, 138.54, 128.50, 124.52, 120.09, 117.44, 117.06, 113.72, 113.31, 112.43, 76.71, 56.34, 56.00, 34.46, 31.60, 29.31, 19.40, 9.78, 9.32.

yy. Preparation of B27

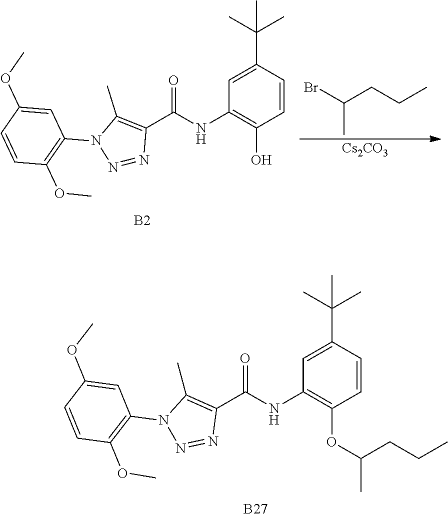

The preparation of compound B27 was prepared by reaction of N-(5-(tert-butyl)-2-hydroxyphenyl)-1-(2,5-dimethoxyphenyl)-5-methyl-1H-1,2,3-triazole-4-carboxamide and 2-bromopentane according to the procedure for B4 synthesis. 98.8 mg, 84% yield, white solid. ¹H NMR (500 MHz, Chloroform-d) δ 9.85 (s, 1H), 8.66 (d, J=2.4 Hz, 1H), 7.08 (dd, J=9.1, 3.0 Hz, 1H), 7.06 (dd, J=8.6, 2.5 Hz, 1H), 7.03 (d, J=9.2 Hz, 1H), 6.96 (d, J=3.0 Hz, 1H), 6.86 (d, J=8.6 Hz, 1H), 4.43 (h, J=6.1 Hz, 1H), 3.81 (s, 3H), 3.76 (s, 3H), 2.54 (s, 3H), 1.93-1.82 (m, 1H), 1.70-1.60 (m, 1H), 1.57-1.45 (m, 2H), 1.37 (d, J=6.1 Hz, 3H), 1.36 (s, 9H), 0.96 (t, J=7.3 Hz, 3H). ¹³C NMR (126 MHz, CDCl3) δ 159.40, 153.68, 148.02, 144.71, 143.80, 139.06, 138.54, 128.48, 124.52, 120.10, 117.43, 117.07, 113.74, 113.31, 112.37, 75.36, 56.34, 56.00, 38.68, 34.46, 31.60, 19.92, 18.79, 14.14, 9.32.

zz. Preparation of B28

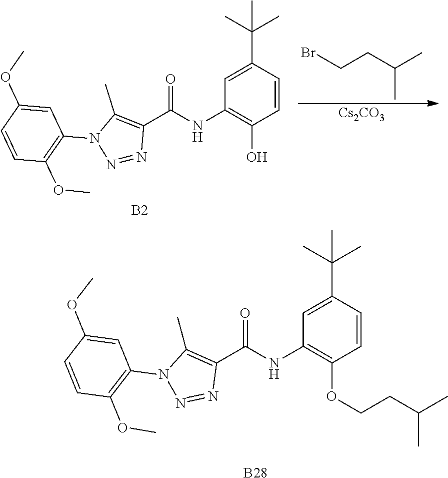

The preparation of compound B28 was prepared by reaction of N-(5-(tert-butyl)-2-hydroxyphenyl)-1-(2,5-dimethoxyphenyl)-5-methyl-1H-1,2,3-triazole-4-carboxamide and 1-bromo-3-methylbutane according to the procedure for B4 synthesis. 88.1 mg, 75% yield, white solid. ¹H NMR (500 MHz, Chloroform-d) δ 9.81 (s, 1H), 8.65 (d, J=2.4 Hz, 1H), 7.10-7.06 (m, 2H), 7.03 (d, J=9.1 Hz, 1H), 6.97 (d, J=3.0 Hz, 1H), 6.86 (d, J=8.6 Hz, 1H), 4.10 (t, J=6.6 Hz, 2H), 3.82 (s, 3H), 3.76 (s, 3H), 2.54 (s, 3H), 1.96 (dp, J=13.4, 6.7 Hz, 1H), 1.80 (q, J=6.7 Hz, 2H), 1.36 (s, 9H), 1.00 (d, J=6.7 Hz, 6H). ¹³C NMR (126 MHz, CDCl3) δ 159.44, 153.68, 148.04, 145.72, 143.75, 139.08, 138.48, 127.51, 124.50, 120.07, 117.45, 117.03, 113.76, 113.31, 110.45, 67.26, 56.34, 56.00, 38.00, 34.45, 31.60, 25.23, 22.69, 9.31.

aaa. Preparation of B29

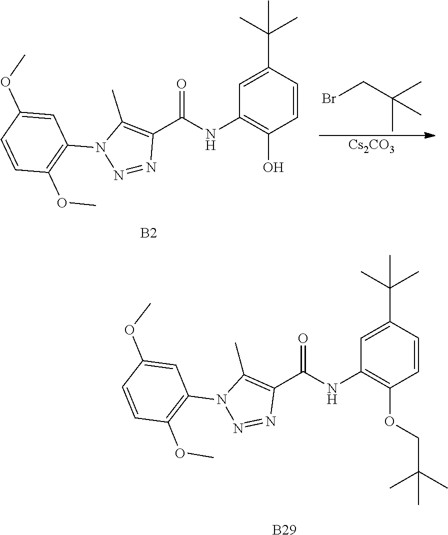

The preparation of compound B29 was prepared by reaction of N-(5-(tert-butyl)-2-hydroxyphenyl)-1-(2,5-dimethoxyphenyl)-5-methyl-1H-1,2,3-triazole-4-carboxamide and 1-bromo-2,2-dimethylpropane according to the procedure for B4 synthesis. 34.2 mg, 29% yield, white solid. ¹H NMR (500 MHz, CDCl3) δ 9.98 (s, 1H), 8.67 (d, J=2.4 Hz, 1H), 7.10-7.05 (m, 2H), 7.02 (d, J=9.1 Hz, 1H), 6.97 (d, J=3.0 Hz, 1H), 6.84 (d, J=8.5 Hz, 1H), 3.81 (s, 3H), 3.76 (s, 3H), 3.71 (s, 2H), 2.54 (s, 3H), 1.36 (s, 9H), 1.15 (s, 9H). ¹³C NMR (126 MHz, CDCl3) δ 159.41, 153.66, 147.99, 145.80, 143.75, 138.99, 138.49, 127.58, 124.46, 119.97, 117.55, 116.76, 113.65, 113.25, 110.17, 78.49, 56.31, 56.01, 34.47, 32.09, 31.60, 26.78, 9.29.

bbb. Preparation of B30

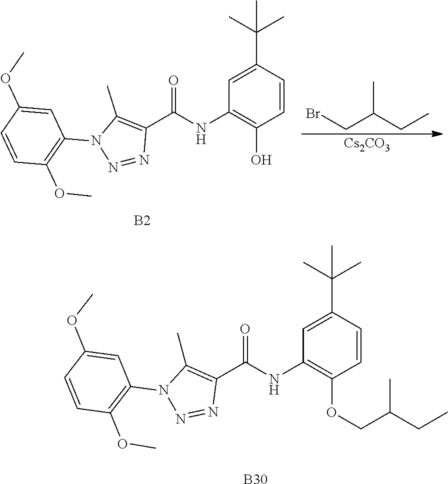

The preparation of compound B30 was prepared by reaction of N-(5-(tert-butyl)-2-hydroxyphenyl)-1-(2,5-dimethoxyphenyl)-5-methyl-1H-1,2,3-triazole-4-carboxamide and 1-bromo-2-methylbutane according to the procedure for B4 synthesis. 94.2 mg, 81% yield, white solid. ¹H NMR (500 MHz, Chloroform-d) δ 9.88 (s, 1H), 8.66 (d, J=2.4 Hz, 1H), 7.10-7.05 (m, 2H), 7.02 (d, J=9.1 Hz, 1H), 6.97 (d, J=3.0 Hz, 1H), 6.85 (d, J=8.5 Hz, 1H), 3.95 (dd, J=8.9, 5.6 Hz, 1H), 3.87 (dd, J=8.8, 6.4 Hz, 1H), 3.81 (s, 3H), 3.76 (s, 3H), 2.54 (s, 3H), 2.04-1.94 (m, 1H), 1.71-1.61 (m, 1H), 1.45-1.37 (m, 1H), 1.36 (s, 9H). 1.13 (d, J=6.8 Hz, 3H). 0.98 (t. J=7.5 Hz, 3H). ¹³C NMR (126 MHz, CDCl3) δ 159.43, 153.67, 148.01, 145.77, 143.73, 139.04, 138.49, 127.57, 124.49, 120.03, 117.48, 116.90, 113.72, 113.29, 110.36, 73.37, 56.32, 56.00, 34.83, 34.46, 31.60, 26.20, 16.78, 11.42, 9.30.

ccc. Preparation of B31

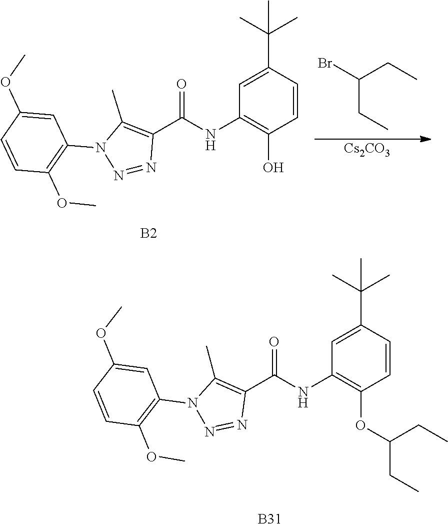

The preparation of compound B31 was prepared by reaction of N-(5-(tert-butyl)-2-hydroxyphenyl)-1-(2,5-dimethoxyphenyl)-5-methyl-1H-1,2,3-triazole-4-carboxamide and 3-bromopentane according to the procedure for B4 synthesis. 84.6 mg, 72% yield, white solid. ¹H NMR (500 MHz, CDCl3) δ 9.90 (s, 1H), 8.66 (d, J=2.4 Hz, 1H), 7.08 (dd, J=9.1, 3.0 Hz, 1H), 7.05 (dd, J=8.6, 2.5 Hz, 1H), 7.03 (d, J=9.1 Hz, 1H), 6.97 (d, J=3.0 Hz, 1H), 6.85 (d, J=8.6 Hz, 1H), 4.21 (p, J=5.8 Hz, 1H), 3.81 (s, 3H), 3.76 (s, 3H), 2.54 (s, 3H), 1.87-1.69 (m, 4H), 1.36 (s, 9H), 1.01 (t, J=7.4 Hz, 6H). ¹³C NMR (126 MHz, CDCl3) δ 159.41, 153.67, 148.00, 145.00, 143.65, 139.04, 138.56, 128.41, 124.53, 120.04, 117.46, 117.00, 113.70, 113.30, 112.14, 81.62, 56.33, 56.00, 34.45, 31.60, 26.03, 9.63, 9.32.

ddd. Preparation of B32

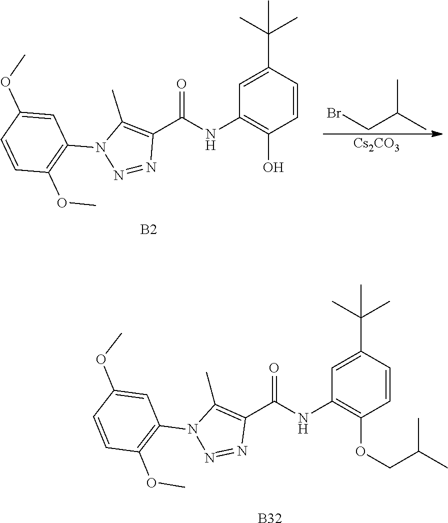

The preparation of compound B32 was prepared by reaction of N-(5-(tert-butyl)-2-hydroxyphenyl)-1-(2,5-dimethoxyphenyl)-5-methyl-1H-1,2,3-triazole-4-carboxamide and 1-bromo-2-methylpropane according to the procedure for B4 synthesis. 83.2 mg, 73% yield, white solid. ¹H NMR (500 MHz, CDCl3) δ 9.89 (s, 1H), 8.66 (d, J=2.4 Hz, 1H), 7.07 (td, J=8.7, 8.2, 2.7 Hz, 2H), 7.02 (d, J=9.2 Hz, 1H), 6.97 (d, J=3.0 Hz, 1H), 6.84 (d, J=8.5 Hz, 1H), 3.84 (d, J=6.4 Hz, 2H), 3.81 (s, 3H), 3.76 (s, 3H), 2.54 (s, 3H), 1.36 (s, 9H), 1.12 (d, J=6.7 Hz, 6H). ¹³C NMR (126 MHz, CDCl₃) δ 159.42, 153.67, 148.01, 145.72, 143.76, 139.04, 138.50, 127.55, 124.50, 120.03, 117.48, 116.92, 113.70, 113.29, 110.40, 75.03, 56.33, 56.00, 34.46, 31.60, 28.41, 19.35, 9.31.

eee. Preparation of B33

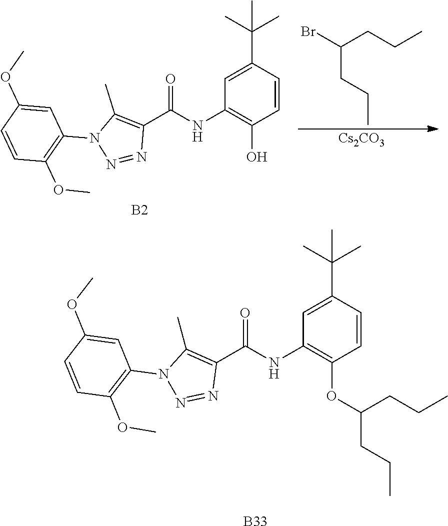

The preparation of compound B33 was prepared by reaction of N-(5-(tert-butyl)-2-hydroxyphenyl)-1-(2,5-dimethoxyphenyl)-5-methyl-1H-1,2,3-triazole-4-carboxamide and 4-bromoheptane according to the procedure for B4 synthesis. 92.3 mg, 74% yield, white solid. ¹H NMR (500 MHz, CDCl3) δ 9.87 (s, 1H), 8.66 (d, J=2.4 Hz, 1H), 7.08 (dd, J=9.1, 3.0 Hz, 1H), 7.06-7.01 (m, 2H), 6.97 (d, J=3.0 Hz, 1H), 6.84 (d, J=8.6 Hz, 1H), 4.33 (p, J=5.9 Hz, 1H), 3.82 (s, 3H), 3.76 (s, 3H), 2.54 (s, 3H), 1.82-1.74 (m, 2H), 1.67 (ddt, J=13.8, 9.8, 5.7 Hz, 2H), 1.54-1.41 (m, 4H), 1.36 (s, 9H), 0.93 (t, J=7.3 Hz, 6H). ¹³C NMR (126 MHz, CDCl3) δ 159.43, 153.68, 148.02, 145.07, 143.55, 139.03, 138.56, 128.32, 124.53, 120.05, 117.45, 117.02, 113.72, 113.31, 111.92, 78.98, 56.34, 56.00, 36.09, 34.44, 31.60, 18.65, 14.23, 9.32.

fff. Preparation of B34

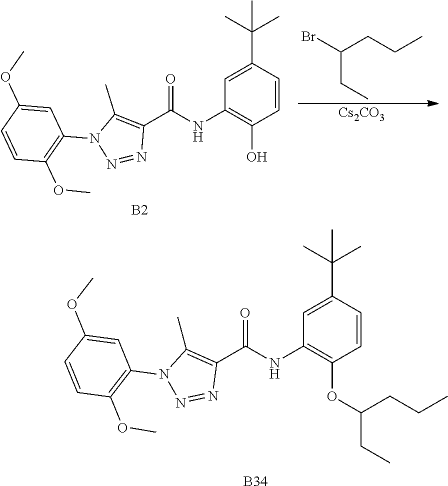

The preparation of compound B34 was prepared by reaction of N-(5-(tert-butyl)-2-hydroxyphenyl)-1-(2,5-dimethoxyphenyl)-5-methyl-1H-1,2,3-triazole-4-carboxamide and 3-bromohexane according to the procedure for B4 synthesis. 95.3 mg, 79% yield, 99.24% purity, white solid. ¹H NMR (500 MHz, CDCl3) δ 9.88 (s, 1H), 8.66 (d, J=2.4 Hz, 1H), 7.08 (dd, J=9.1, 3.0 Hz, 1H), 7.07-7.01 (m, 2H), 6.97 (d, J=3.0 Hz, 1H), 6.84 (d, J=8.6 Hz, 1H), 4.27 (p, J=5.8 Hz, 1H), 3.82 (s, 3H), 3.76 (s, 3H), 2.54 (s, 3H), 1.82-1.63 (m, 4H), 1.53-1.42 (m, 2H), 1.36 (s, 9H), 1.01 (t, J=7.5 Hz, 3H), 0.94 (t, J=7.4 Hz, 3H). ¹³C NMR (126 MHz, CDCl3) δ 159.42, 153.68, 148.01, 145.03, 143.60, 139.04, 138.56, 128.36, 124.53, 120.05, 117.46, 117.01, 113.71, 113.30, 112.03, 80.27, 56.34, 56.01, 35.51, 34.45, 31.60, 26.57, 18.69, 14.23, 9.58, 9.32. ESI-TOF HRMS: m/z 495.2967 (C₂₈H₃₈N₄O₄+H⁺ requires 495.2966).

ggg. Preparation of B35

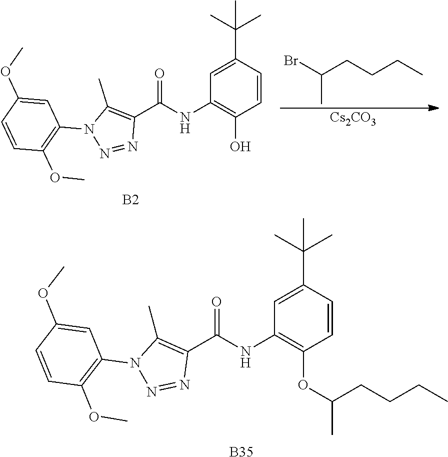

The preparation of compound B35 was prepared by reaction of N-(5-(tert-butyl)-2-hydroxyphenyl)-1-(2,5-dimethoxyphenyl)-5-methyl-1H-1,2,3-triazole-4-carboxamide and 2-chlorohexane according to the procedure for B4 synthesis. 67.3 mg, 56% yield, white solid. ¹H NMR (500 MHz, CDCl3) δ 9.84 (s, 1H), 8.65 (d, J=2.4 Hz, 1H), 7.08 (dd, J=9.1, 3.0 Hz, 1H), 7.05 (dd, J=8.5, 2.4 Hz, 1H), 7.03 (d, J=9.1 Hz, 1H), 6.96 (d, J=3.0 Hz, 1H), 6.86 (d, J=8.6 Hz, 1H), 4.41 (h, J=6.1 Hz, 1H), 3.82 (s, 3H), 3.76 (s, 3H), 2.54 (s, 3H), 1.94-1.83 (m, 1H), 1.73-1.62 (m, 1H), 1.52-1.41 (m, 2H), 1.41-1.33 (m, 14H), 0.91 (t, J=7.2 Hz, 3H). ¹³C NMR (126 MHz, CDCl3) δ 159.42, 153.68, 148.03, 144.73, 143.80, 139.06, 138.53, 128.47, 124.54, 120.11, 117.43, 117.10, 113.75, 113.32, 112.38, 75.66, 56.34, 56.00, 36.24, 34.46, 31.60, 27.75, 22.72, 19.95, 14.10, 9.32.

hhh. Preparation of B36

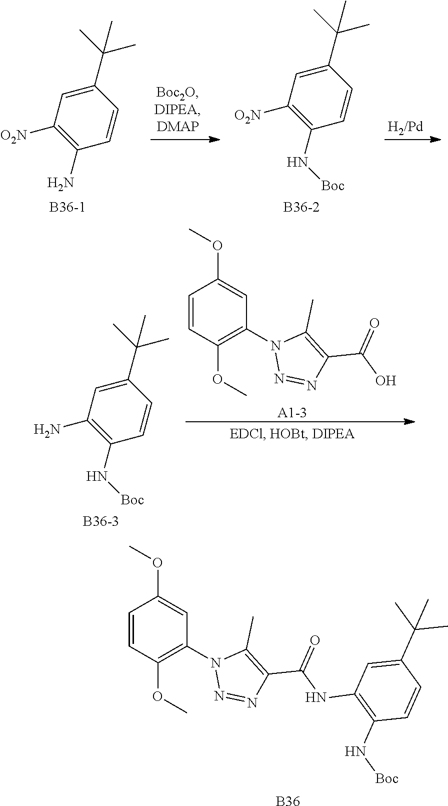

To a solution of 4-(tert-butyl)-2-nitroaniline (5 g, 25.7 mmol), N,N-dimethylpyridin-4-amine (0.314 g, 2.57 mmol), and N-ethyl-N-isopropylpropan-2-amine (6.65 g, 51.5 mmol) in DCM (100 ml) was added di-tert-butyl dicarbonate (6.18 g, 28.3 mmol). The reaction mixture was stirred at room temperature for overnight. The reaction mixture was evaporated to afford the crude product. The residue was added water (100 ml) and extracted with EtOAc (200 ml). The organic phase was washed with NH₄C1 saturated solution. The organic layer was dried over MgSO₄, and concentrated. The residue was purified by silica gel chromatography (0% to 100% EA in hexane) to give product (5.88 g, 78% yield) as a yellow solid. ¹H NMR (400 MHz, Chloroform-d) δ 7.14 (d, J=8.0 Hz, 1H), 6.83-6.78 (m, 2H), 6.13 (s, 1H), 1.51 (s, 9H), 1.27 (s, 9H). ¹³C NMR (101 MHz, CDCl3) δ 154.02, 149.63, 139.64, 124.67, 121.99, 116.80, 114.84, 80.43, 34.32, 31.31, 28.34.

To a solution of tert-butyl (4-(tert-butyl)-2-nitrophenyl)carbamate (5.88 g, 19.98 mmol) in 100 ml MeOH was added Pd/C (1 g, 10%) under N2. The suspension was degassed under vacuum and purged with H₂ several times. The mixture was stirred under H2 balloon at room temperature for 12 hours. Then the reaction mixture was filtrated. The filter was concentrated to obtain tert-butyl (2-amino-4-(tert-butyl)phenyl)carbamate without purified for the next step.

To a solution of 1-(2,5-dimethoxyphenyl)-5-methyl-1H-1,2,3-triazole-4-carboxylic acid (0.526 g, 2 mmol) in DMF 5 mL was added tert-butyl (2-amino-4-(tert-butyl)phenyl)carbamate (0.529 g, 2.000 mmol), HOBt 80% (0.405 g, 2.400 mmol) and EDCI (0.575 g, 3.00 mmol), followed by N-ethyl-N-isopropylpropan-2-amine (0.661 ml, 4.00 mmol). The suspension was stirred at room temperature for overnight. The reaction mixture was then diluted with water (100 mL) and extracted with EA (100 mL×2). The combine EA layers were washed with water(100 mL), dried with anhydrous Na₂SO₄, and concentrated. The residue was purified by silica gel chromatography (0% to 100% EA in hexane) to give product as white solid (891.2 mg, 87% yield). ¹H NMR (400 MHz, Chloroform-d) δ 9.23 (s, 1H), 7.63-7.56 (m, 2H), 7.26 (dd, J=8.6, 2.2 Hz, 1H), 7.10 (dd, J=9.1, 3.0 Hz, 2H), 7.04 (d, J=9.1 Hz, 1H), 6.95 (d, J=3.0 Hz, 1H), 3.82 (s, 3H), 3.78 (s, 3H), 2.52 (s, 3H), 1.52 (s, 9H), 1.32 (s, 9H). ¹³C NMR (101 MHz, CDCl3) δ 160.30, 153.88, 153.72, 148.34, 148.04, 139.65, 137.68, 128.81, 128.60, 124.33(2C), 123.57, 121.61, 117.49, 113.85, 113.35, 80.55, 56.36, 56.00, 34.47, 31.32, 28.36, 9.27. ESI-TOF HRMS: m/z 510.2720 (C₂₇H₃₅N₅O₅+H⁺ requires 510.2711).

iii. Preparation of B37

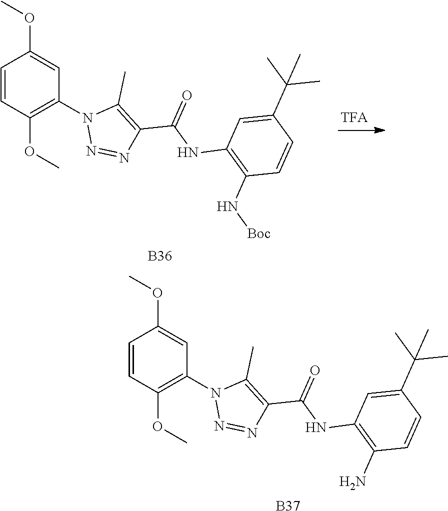

To a solution of tert-butyl (4-(tert-butyl)-2-(1-(2,5-dimethoxyphenyl)-5-methyl-1H-1,2,3-triazole-4-carboxamido)phenyl)carbamate (200 mg, 0.392 mmol) in DCM 10 mL was added 2,2,2-trifluoroacetic acid (10 ml, 0.392 mmol) at ice bath. Then the solution was stirred at room temperature for 1 h. The reaction mixture was then diluted with water (20 mL) and extracted with EA (25 mL×2). The combine EA layers were washed with water (30 mL), dried with anhydrous Na₂SO₄, and concentrated. The residue was purified by silica gel chromatography (0% to 100% EA in hexane) to give product (117.5 mg, 79% yield). ¹H NMR (400 MHz, Chloroform-d) δ 8.98 (s, 1H), 7.40 (d, J=2.2 Hz, 1H), 7.16-7.06 (m, 2H), 7.03 (d, J=9.1 Hz, 1H), 6.95 (d, J=3.0 Hz, 1H), 6.80 (dd, J=8.3, 2.0 Hz, 1H), 3.82 (s, 3H), 3.77 (s, 3H), 2.51 (s, 3H), 1.30 (s, 9H). ¹³C NMR (101 MHz, CDCl3) δ 159.71, 153.71, 148.05, 142.68, 142.66, 139.43, 138.14, 137.75, 124.40, 124.08, 123.47, 122.09, 117.85, 117.46, 113.84, 113.36, 56.36, 56.01, 34.06, 31.50, 9.22. ESI-TOF HRMS: m/z 410.2181 (C₂₂H27N₅O3+H⁺ requires 410.2187).

jjj. Preparation of B38

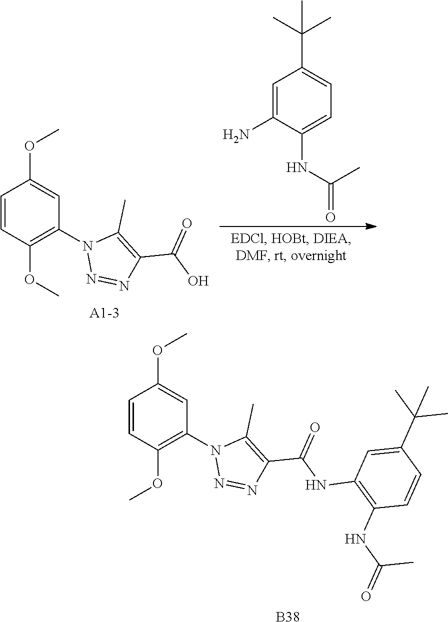

The preparation of compound B38 was prepared by reaction of 1-(2,5-dimethoxyphenyl)-5-methyl-1H-1,2,3-triazole-4-carboxylic acid and N-(2-amino-4-(tert-butyl)phenyl)acetamide according to the procedure for A1 synthesis. White solid, 3.65 g, 81% yield. ¹H NMR (400 MHz, Chloroform-d) δ 9.24 (s. 1H), 8.56 (s, 1H), 7.66 (d, J=8.5 Hz, 1H), 7.46 (d, J=2.2 Hz, 1H), 7.28 (dd, J=8.5, 2.3 Hz, 1H), 7.09 (dd, J=9.1, 2.9 Hz, 1H), 7.04 (d, J=9.1 Hz, 1H), 6.95 (d, J=3.0 Hz, 1H), 3.81 (s, 3H), 3.77 (s, 3H), 2.50 (s, 3H), 2.16 (s, 3H), 1.31 (s, 9H). ¹³C NMR (101 MHz, CDCl3) δ 167.10, 158.55, 151.84, 147.23, 146.11, 137.85, 135.51, 126.85, 126.53, 123.68, 122.32, 121.90, 119.42, 115.67, 111.96, 111.49, 54.48, 54.13, 32.65, 29.40, 22.33, 7.38. ESI-TOF HRMS: m/z 452.2290 (C₂₄H₂₉N₅O₄+H⁺ requires 452.2293).

kkk. Preparation of C1

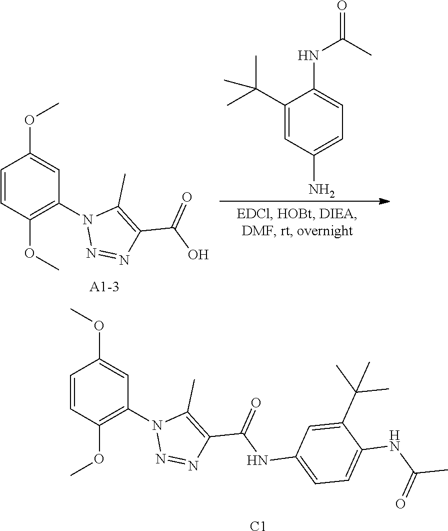

The preparation of compound C1 was prepared by reaction of 1-(2,5-dimethoxyphenyl)-5-methyl-1H-1,2,3-triazole-4-carboxylic acid and N-(4-amino-2-(tert-butyl)phenyl)acetamide according to the procedure for A1 synthesis. White solid, 341.0 mg, 75% yield. ¹H NMR (400 MHz, DMSO-d₆) δ 10.44 (s, 1H), 9.25 (s, 1H), 7.64 (h, J=2.4 Hz, 2H), 7.33 (d, J=9.4 Hz, 1H), 7.28 (d, J=9.2 Hz, 1H), 7.22 (dd, J=9.1, 3.0 Hz, 1H), 7.15 (d, J=3.0 Hz, 1H), 3.78 (s, 3H), 3.75 (s, 3H), 2.38 (s, 3H), 2.04 (s, 3H), 1.31 (s, 9H). ¹³C NMR (101 MHz, DMSO) δ 168.98, 159.35, 153.05, 147.69, 141.79, 138.95, 137.51, 136.80, 136.01, 126.50, 123.69, 123.42, 118.67, 117.39, 113.99, 113.85, 56.30, 55.85, 34.39, 30.80, 23.11, 8.77. ESI-TOF HRMS: m/z 452.2288 (C₂₄H₂₉N₅O₄+H⁺ requires 452.2293).

lll. PREPARATION C2

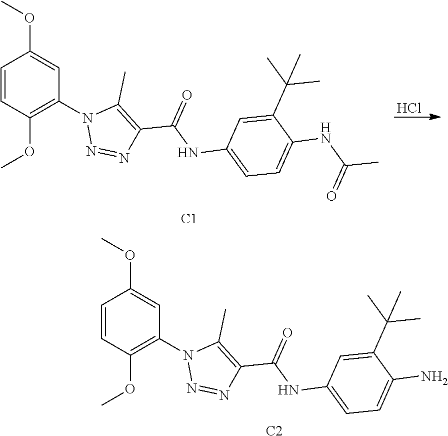

To a solution of N-(4-acetamido-3-(tert-butyl)phenyl)-1-(2,5-dimethoxyphenyl)-5-methyl-1H-1,2,3-triazole-4-carboxamide (0.135 g, 0.3 mmol) in water (10 mL) and EtOH (5 mL) was added 12 M hydrochloric acid (2 mL, 6 mmol). The suspension was stirred 80° C. for overnight. After the reaction mixture was cool down, the pH was adjusted to around 8 by NaHCO₃ saturated solution. Then the mixture was extracted with EtOAc (100 mL). The organic solvent washed by NaHCO₃ saturated solution (50 mL), water (50 mL) and brine (50 mL). The organic phase was dried over MgSO₄, filtered and concentrated. The residue was purified to provide N-(4-amino-3-(tert-butyl)phenyl)-1-(2,5-dimethoxyphenyl)-5-methyl-1H-1,2,3-triazole-4-carboxamide (78.5 mg, 64% yield) as a white solid. ¹H NMR (400 MHz, DMSO-d₆) δ 9.99 (s, 1H), 7.28 (d, J=9.1 Hz, 1H), 7.22 (dt, J=6.2, 3.4 Hz, 2H), 7.13 (d, J=2.9 Hz, 1H), 7.00 (d, J=8.5 Hz, 1H), 6.89 (dd, J=8.5, 2.2 Hz, 1H), 4.77 (s, 2H), 3.78 (s, 3H), 3.75 (s, 3H), 2.37 (s, 3H), 1.33 (s, 9H). ¹³C NMR (101 MHz, DMSO) δ 159.01, 153.04, 147.70, 146.01, 138.63, 137.73, 136.78, 128.04, 125.90, 123.76, 117.33, 114.01, 113.84, 109.01, 108.91, 56.29, 55.84, 33.58, 29.37, 8.73.

ESI-TOF HRMS: m/z 410.2183 (C₂₂H₂₇N₅O₃+H⁺ requires 410.2187).

mmm. Preparation of C3

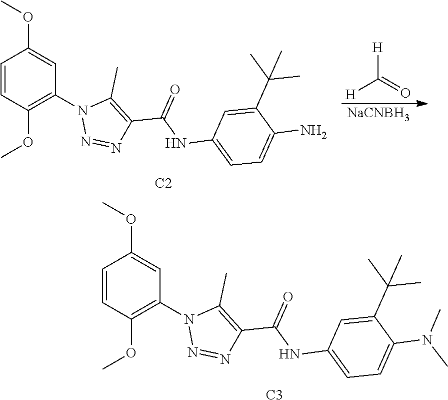

N-(4-amino-3-(tert-butyl)phenyl)-1-(2,5-dimethoxyphenyl)-5-methyl-1H-1,2,3-triazole-4-carboxamide (0.205 g, 0.5 mmol), formaldehyde (0.150 g, 5.00 mmol) and sodium cyanoborohydride (0.094 g, 1.500 mmol) were added into MeOH (5 mL). The reaction mixture was stirred at room temperature for 12 hours. The reaction mixture was diluted with water (30 mL) and extracted with EA (25 mL×2). The combine EA layers were washed with water (30 mL), dried with anhydrous Na₂SO₄, and concentrated. The residue was purified by reverse silica gel chromatography (0% to 100% acetonitrile in water) to give product as a white solid (143.2 mg, 65% yield). ¹H NMR (400 MHz, Chloroform-d) δ 9.02 (s, 1H), 7.77 (d, J=2.3 Hz, 1H), 7.39 (dd, J=8.6, 2.3 Hz, 1H), 7.34 (d, J=8.6 Hz, 1H), 7.09 (dd, J=9.1, 3.0 Hz, 1H), 7.03 (d, J=9.1 Hz, 1H), 6.95 (d, J=2.9 Hz, 1H), 3.82 (s, 3H), 3.77 (s, 3H), 2.64 (s, 6H), 2.52 (s, 3H), 1.44 (s, 9H). ¹³C NMR (101 MHz, CDCl3) δ 159.28, 155.72, 153.72, 148.04, 143.39, 139.37, 137.95, 136.66, 127.25, 124.36, 117.50, 117.01, 116.81, 113.83, 113.35, 56.34, 56.01, 47.11, 35.22, 30.94, 9.24. ESI-TOF HRMS: m/z 438.2498 (C₂₄H₃₁N₅O₃+H⁺ requires 438.2500).

nnn. Preparation of C4

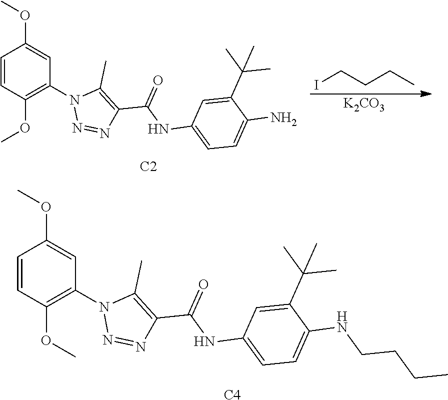

N-(4-amino-3-(tert-butyl)phenyl)-1-(2,5-dimethoxyphenyl)-5-methyl-H-1,2,3-triazole-4-carboxamide (0.205 g, 0.5 mmol), potassium carbonate (0.138 g, 1.000 mmol) and 1-iodobutane (0.057 ml, 0.500 mmol) were added into DMF (5 mL). The reaction mixture was stirred at room temperature for 12 hours. The reaction mixture was then diluted with water (30 mL) and extracted with EtOAc (25 mL×2). The combine EtOAc layers were washed with water (30 mL), dried with anhydrous Na₂SO₄, and concentrated. The residue was purified by silica gel chromatography (0% to 100% EA in hexane) to give product as a white solid (148.9 mg, 64% yield). ¹H NMR (400 MHz, Chloroform-d) δ 8.98 (s, 1H), 7.20 (d, J=8.4 Hz, 1H), 7.11-7.05 (m, 2H), 7.03 (d, J=9.1 Hz, 1H), 6.99 (dd, J=8.4, 2.3 Hz, 1H), 6.95 (d, J=3.0 Hz, 1H), 3.82 (s, 3H), 3.76 (s, 3H), 3.22 (t, J=6.9 Hz, 2H), 2.52 (s, 3H), 1.84-1.61 (m, 2H), 1.56-1.47 (m, 2H), 1.42 (s, 9H), 1.00 (t, J=7.3 Hz, 3H). ¹³C NMR (101 MHz, CDCl3) δ 159.20, 153.70, 148.07, 147.09, 139.24, 138.16, 137.00, 129.24, 126.65, 124.43, 117.47, 113.83, 113.34, 107.81, 103.13, 56.34, 56.01, 44.13, 33.89, 31.70, 30.05, 20.60, 13.97, 9.25. ESI-TOF HRMS: m/z 466.2810 (C₂₆H₃₅N₅O₃+H⁺ requires 466.2813).

ooo. Preparation of C5

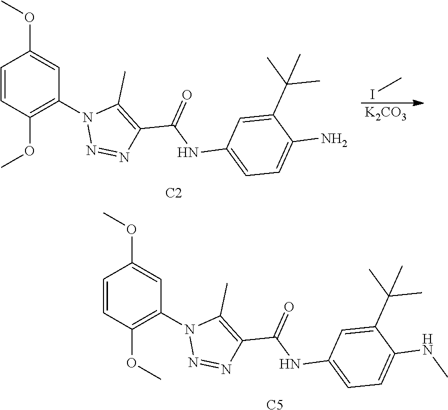

The preparation of compound C5 was prepared by reaction of 1 N-(4-amino-3-(tert-butyl)phenyl)-1-(2,5-dimethoxyphenyl)-5-methyl-1H-1,2,3-triazole-4-carboxamide and iodomethane according to the procedure for C4 synthesis. White solid, 142.1 mg, 67% yield. ¹H NMR (400 MHz, Chloroform-d) δ 9.01 (s, 1H), 7.21 (d, J=8.4 Hz, 1H), 7.12-7.06 (m, 2H), 7.05-6.99 (m, 2H), 6.95 (d, J=3.0 Hz, 1H), 3.81 (s, 3H), 3.76 (s, 3H), 2.95 (s, 3H), 2.52 (s, 3H), 1.41 (s, 9H). ¹³C NMR (101 MHz, CDCl3) δ 159.22, 153.70, 148.06, 147.99, 139.26, 138.15, 137.08, 129.46, 126.58, 124.42, 117.46, 113.84, 113.35, 107.89, 102.74, 56.34, 56.00, 33.88, 31.20, 29.98, 9.25. ESI-TOF HRMS: m/z 424.2343 (C₂₃H₂₉N₅O₃+H⁺ requires 424.2343).

ppp. Preparation of C6

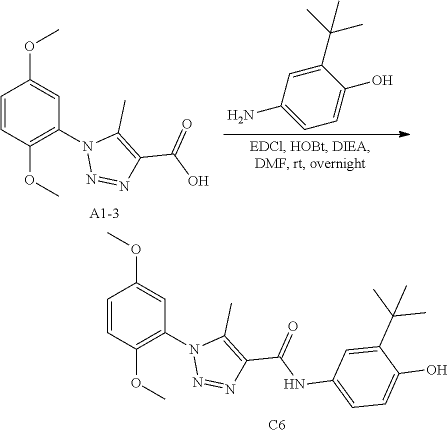

The preparation of compound C6 was prepared by reaction of 1-(2,5-dimethoxyphenyl)-5-methyl-1H-1,2,3-triazole-4-carboxylic acid and 4-amino-2-(tert-butyl)phenol according to the procedure for A1 synthesis. White solid, 579.2 mg, 47% yield. ¹H NMR (400 MHz, DMSO-d6) δ 10.14 (s, 1H), 9.17 (s, 1H), 7.61 (d, J=2.6 Hz, 1H), 7.52 (dd, J=8.5, 2.5 Hz, 1H), 7.27 (d, J=9.2 Hz, 1H), 7.21 (dd, J=9.1, 3.1 Hz, 1H), 7.13 (d, J=3.0 Hz, 1H), 6.73 (d, J=8.6 Hz, 1H), 3.78 (s, 3H), 3.75 (s, 3H), 2.37 (s, 3H), 1.36 (s, 9H). 13C NMR (101 MHz, DMSO) δ 158.92, 153.05, 152.16, 147.69, 138.45, 137.80, 135.13, 129.89, 123.80, 119.83, 119.29, 117.30, 115.62, 113.99, 113.83, 56.28, 55.84, 34.39, 29.30, 8.74. ESI-TOF HRMS: m/z 411.2021 (C₂₂H₂₆N₄O₄+H⁺ requires 411.2027).

qqq. Preparation of C7

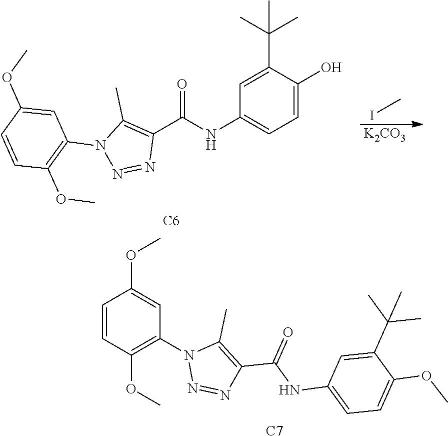

To a solution of N-(3-(tert-butyl)-4-hydroxyphenyl)-1-(2,5-dimethoxyphenyl)-5-methyl-1H-1,2,3-triazole-4-carboxamide (100 mg, 0.244 mmol) in DMF 5 mL was added iodomethane (34.6 mg, 0.244 mmol), and K₂CO₃ (67.3 mg, 0.487 mmol). The suspension was stirred at room temperature for overnight. Then the reaction mixture was then diluted with water (25 mL) and extracted with EtOAc (25 mL×2). The combine EtOAc layers were washed with water (20 mL), dried with anhydrous Na₂SO₄, and concentrated. The residue was purified by silica gel chromatography (0% to 100% EtOAc in hexane) to give product as a white solid (84.2 mg, 82% yield). ¹H NMR (400 MHz, Chloroform-d) δ 8.96 (s, 1H), 7.66 (dd, J=8.7, 2.7 Hz, 1H), 7.45 (d, J=2.7 Hz, 1H), 7.09 (dd, J=9.1, 3.0 Hz, 1H), 7.03 (d, J=9.1 Hz, 1H), 6.95 (d, J=3.0 Hz, 1H), 6.88 (d, J=8.8 Hz, 1H), 3.85 (s, 3H), 3.82 (s, 3H), 3.76 (s, 3H), 2.52 (s, 3H), 1.40 (s, 9H). ¹³C NMR (101 MHz, CDCl3) δ 159.21, 155.44, 153.70, 148.06, 139.20, 138.97, 138.07, 130.44, 124.42, 119.30, 118.77, 117.46, 113.83, 113.34, 111.94, 56.34, 56.01, 55.36, 34.94, 29.67, 9.22. ESI-TOF HRMS: m/z 425.2190 (C₂₃H₂₈N₄O₄+H⁺ requires 425.2184).

rrr. Preparation of C8

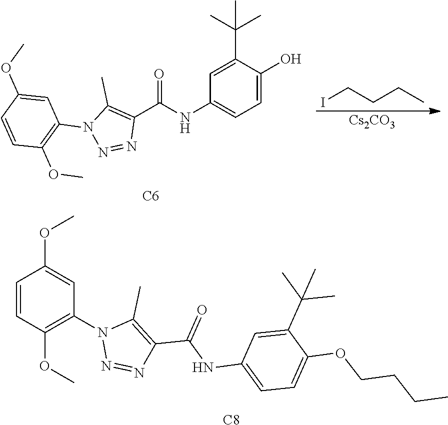

To a solution of N-(3-(tert-butyl)-4-hydroxyphenyl)-1-(2,5-dimethoxyphenyl)-5-methyl-1H-1,2,3-triazole-4-carboxamide (0.164 g, 0.4 mmol) in DMF 5 mL was added 1-iodobutane (0.088 g, 0.480 mmol), and Cs₂CO₃ (0.156 g, 0.480 mmol). The suspension was stirred overnight. Then the reaction mixture was then diluted with water (20 mL) and extracted with EtOAc (25 mL×2). The combine EtOAc layers were washed with water (20 mL), dried with anhydrous Na₂SO₄, and concentrated. The residue was purified by silica gel chromatography (0% to 100% EtOAc in hexane) to give product (138.6 mg, 74% yield). ¹H NMR (400 MHz, Chloroform-d) δ 8.95 (s, 1H), 7.64 (dd, J=8.7, 2.7 Hz, 1H), 7.45 (d, J=2.7 Hz, 1H), 7.09 (dd, J=9.1, 3.0 Hz, 1H), 7.03 (d, J=9.2 Hz, 1H), 6.95 (d, J=3.0 Hz, 1H), 6.86 (d, J=8.8 Hz, 1H), 3.99 (t, J=6.4 Hz, 2H), 3.82 (s, 3H), 3.76 (s, 3H), 2.52 (s, 3H), 1.84 (dq, J=8.6, 6.5 Hz, 2H), 1.59-1.51 (m, 2H), 1.41 (s, 9H), 1.00 (t, J=7.4 Hz, 3H). ¹³C NMR (101 MHz, CDCl3) δ 159.18, 154.82, 153.70, 148.07, 139.17, 138.68, 138.10, 130.12, 124.43, 119.34, 118.73, 117.46, 113.83, 113.34, 112.05, 67.76, 56.34, 56.01, 34.98, 31.62, 29.74, 19.63, 13.90, 9.22. ESI-TOF HRMS: m/z 467.2645 (C26H34N₄O₄+H⁺ requires 467.2653).

sss. Preparation of D1

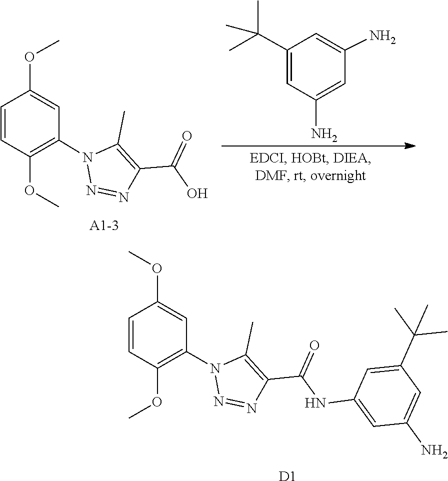 [SMILES: COc1ccc(OC)c(-n2nnc(C(=O)O)c2C)c1 ; CC(C)(C)c1cc(N)cc(N)c1 ; COc1ccc(OC)c(-n2nnc(C(=O)Nc3cc(N)cc(C(C)(C)C)c3)c2C)c1 ; "D1"]

The preparation of compound D1 was prepared by reaction of 1-(2,5-dimethoxyphenyl) -5-methyl-1H-1,2,3-triazole-4-carboxylic acid and 5-(tert-butyl)benzene-1,3-diamine according to the procedure for A1 synthesis. Brown solid, 1.33 g, 65% yield. ¹H NMR (400 MHz, Chloroform-d) δ 9.00 (s, 1H), 7.25 (t, J=2.0 Hz, 1H), 7.09 (dd, J=9.2, 3.0 Hz, 1H), 7.03 (d, J=9.1 Hz, 1H), 6.95 (d, J=3.0 Hz, 1H), 6.92 (t, J=1.8 Hz, 1H), 6.53 (t, J=1.8 Hz, 1H), 3.82 (s, 3H), 3.76 (s, 3H), 2.51 (s, 3H), 1.30 (s, 9H). ¹³C NMR (101 MHz, CDCl3) δ 159.32, 153.70, 153.36, 148.04, 146.57, 139.37, 138.47, 138.02, 124.36, 117.50, 113.81, 113.34, 108.67, 107.63, 104.16, 56.34, 56.01, 34.70, 31.25, 9.23. ESI-TOF HRMS: m/z 410.2190 (C₂₂H₂₇N₅O₃+H⁺ requires 410.2187).

ttt. Preparation of D2 and D3

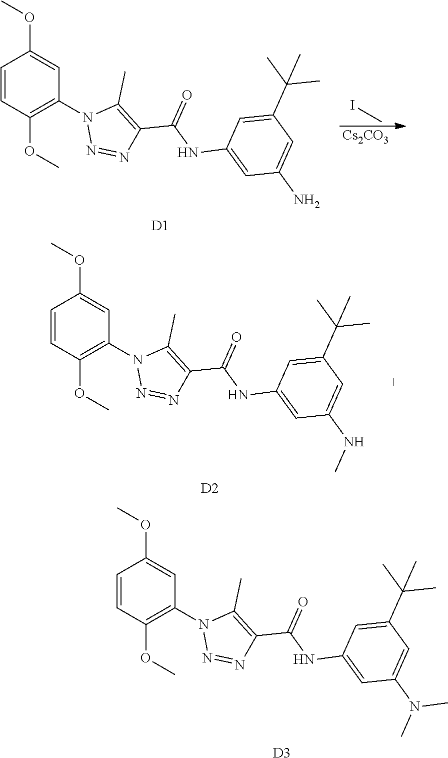

To a solution of N-(3-amino-5-(tert-butyl)phenyl)-1-(2,5-dimethoxyphenyl)-5-methyl-1H-1,2,3-triazole-4-carboxamide (0.205 g, 0.5 mmol) in DMF 5 mL was added iodomethane (0.300 ml, 0.600 mmol), and Cs₂CO₃ (0.195 g, 0.600 mmol). The suspension was stirred at room temperature for overnight. Then the reaction mixture was diluted with water (20 mL) and extracted with EtOAc (25 mL×2). The combine EtOAc layers were washed with water (20 mL), dried with anhydrous Na₂SO₄, and concentrated. The residue was purified by silica gel chromatography (0% to 100% EA in hexane) to give product N-(3-(tert-butyl)-5-(methylamino)phenyl)-1-(2,5-dimethoxyphenyl)-5-methyl-1H-1,2,3-triazole-4-carboxamide (102.5 mg, 48% yield), ¹H NMR (400 MHz, Chloroform-d) δ 9.02 (s, 1H), 7.11 (t, J=2.0 Hz, 1H), 7.10-7.07 (m, 1H), 7.03 (d, J=9.1 Hz, 1H), 6.97-6.94 (m, 2H), 6.46 (t, J=1.9 Hz, 1H), 3.82 (s, 3H), 3.76 (s, 3H), 2.89 (s, 3H), 2.52 (s, 3H), 1.32 (s, 9H). ¹³C NMR (101 MHz, CDCl3) δ 159.30, 153.70, 153.08, 149.59, 148.06, 139.29, 138.60, 138.11, 124.40, 117.49, 113.82, 113.34, 106.59, 106.41, 101.20, 56.34, 56.01, 34.79, 31.31, 31.03, 9.26. ESI-TOF HRMS: m/z 424.2346 (C₂₃H₂₉N₅O₃+H⁺ requires 424.2343).

N-(3-(tert-butyl)-5-(dimethylamino)phenyl)-1-(2,5-dimethoxyphenyl)-5-methyl-1H-1,2,3-triazole-4-carboxamide (79.2 mg, 34% yield), ¹H NMR (400 MHz, Chloroform-d) δ 9.04 (s, 1H), 7.17 (s, 1H), 7.09 (dd, J=9.1, 3.0 Hz, 1H), 7.06-7.00 (m, 2H), 6.96 (d, J=3.0 Hz, 1H), 6.58 (s, 1H), 3.82 (s, 3H), 3.76 (s, 3H), 3.00 (s, 6H), 2.53 (s, 3H), 1.35 (s, 9H). ¹³C NMR (101 MHz, CDCl3) δ 159.30, 153.70, 152.80, 151.07, 148.06, 139.24, 138.44, 138.17, 124.41, 117.48, 113.83, 113.35, 106.21, 105.95, 101.75, 56.34, 56.02, 40.89, 35.01, 31.41, 9.28. ESI-TOF HRMS: m/z 438.2501 (C₂₄H₃₁N5O₃+H⁺ requires 438.2500).

uuu. Preparation of D4 and D5

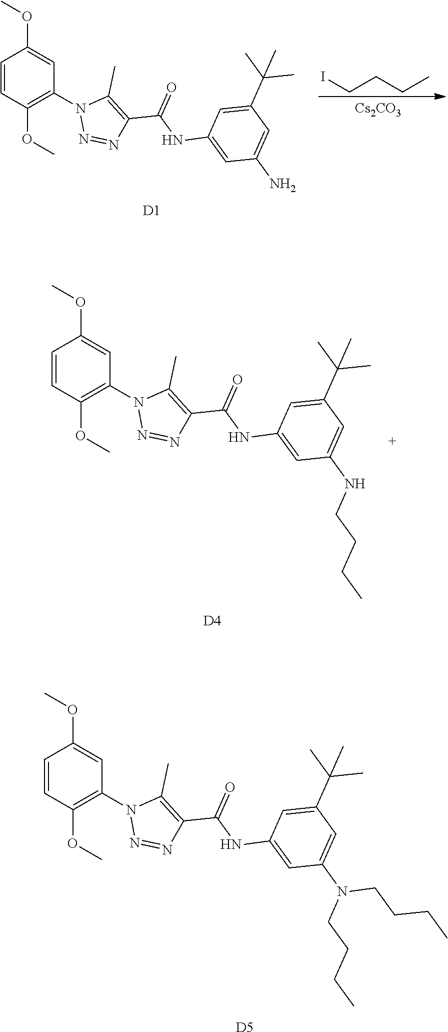

The preparation of compound D4 and D5 was prepared by reaction of N-(3-amino-5-(tert-butyl)phenyl)-1-(2,5-dimethoxyphenyl)-5-methyl-1H-1,2,3-triazole-4-carboxamide and 1-iodobutane to the procedure for D2 and D3 synthesis. N-(3-(tert-butyl)-5-(butylamino)phenyl)-1-(2,5-dimethoxyphenyl)-5-methyl-1H-1,2,3-triazole-4-carboxamide (89.6 mg, 48% yield), ¹H NMR (400 MHz, Chloroform-d) δ 8.98 (s, 1H), 7.12-7.00 (m, 3H), 6.95 (d, J=3.0 Hz, 1H), 6.92 (t, J=2.1 Hz, 1H), 6.48 (dd, J=2.4, 1.6 Hz, 1H), 3.82 (s, 3H), 3.76 (s, 3H), 3.34-3.26 (m, 4H), 2.53 (s, 3H), 1.65-1.56 (m, 4H), 1.45-1.36 (m, 4H), 1.34 (s, 9H), 0.98 (t, J=7.3 Hz, 6H). ¹³C NMR (101 MHz, CDCl3) δ 159.19, 153.70, 152.68, 148.60, 148.08, 139.19, 138.55, 138.29, 124.45, 117.44, 113.84, 113.35, 105.49, 104.77, 100.83, 56.34, 56.01, 50.96, 34.96, 31.40, 29.56, 20.43, 14.07, 9.28. ESI-TOF HRMS: m/z 466.2807 (C₂₆H₃₅N₅O₃+H⁺ requires 466.2813).

N-(3-(tert-butyl)-5-(dibutylamino)phenyl)-1-(2,5-dimethoxyphenyl)-5-methyl-1H-1,2,3-triazole-4-carboxamide (45.9 mg, 22% yield), ¹H NMR (400 MHz, Chloroform-d) δ 9.00 (s, 1H), 7.13-7.06 (m, 2H), 7.03 (d, J=9.1 Hz, 1H), 6.95 (d, J=3.0 Hz, 1H), 6.92 (t, J=1.7 Hz, 1H), 6.44 (t, J=1.9 Hz, 1H), 3.82 (s, 3H), 3.76 (s, 3H), 3.16 (t, J=7.1 Hz, 2H), 2.52 (s, 3H), 1.68-1.56 (m, 2H), 1.52-1.40 (m, 2H), 1.31 (s, 9H), 0.97 (t, J=7.3 Hz, 3H). ¹³C NMR (101 MHz, CDCl3) δ 159.27, 153.70, 153.05, 148.87, 148.06, 139.27, 138.57, 138.13, 124.41, 117.48, 113.82, 113.34, 106.59, 106.34, 101.39, 56.34, 56.01, 43.89, 34.77, 31.71, 31.31, 20.33, 13.96, 9.25. ESI-TOF HRMS: m/z 522.3427 (C₃₀H₄₃N₅O₃+H⁺ requires 522.3439).

vvv. Preparation of D6

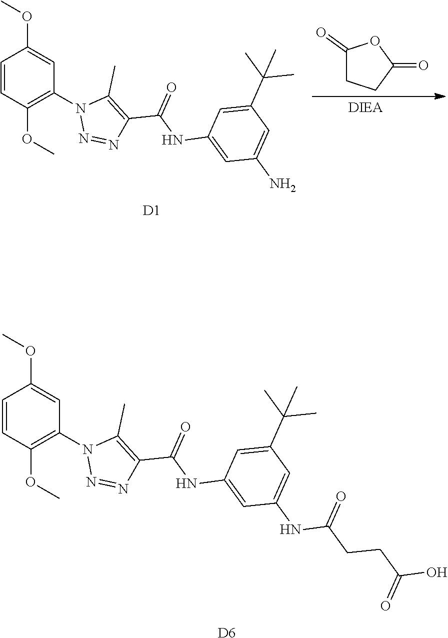

To a solution of N-(3-amino-5-(tert-butyl)phenyl)-1-(2,5-dimethoxyphenyl)-5-methyl-1H-1,2,3-triazole-4-carboxamide (200 mg, 0.488 mmol) in DMF 5 mL was added dihydrofuran-2,5-dione (58.7 mg, 0.586 mmol) and N-ethyl-N-isopropylpropan-2-amine (126 mg, 0.977 mmol). The suspension was stirred at room temperature for 3 hours. Then the reaction mixture was diluted with water (20 mL) and extracted with EtOAc (25 mL×2). The combine EtOAc layers were washed with water (20 mL), dried with anhydrous Na₂SO₄, and concentrated. The residue was purified by silica gel chromatography (0% to 100% EA in hexane) to give product (188.9 mg, 76% yield). ¹H NMR (400 MHz, DMSO-d6) δ 12.11 (s, 1H), 10.32 (s, 1H), 9.93 (s, 1H), 8.09 (s, 1H), 7.48 (d, J=12.7 Hz, 2H), 7.32-7.11 (m, 3H), 3.78 (s, 3H), 3.75 (s, 3H), 2.63-2.51 (m, 4H), 2.39 (s, 3H), 1.27 (s, 9H). ¹³C NMR (101 MHz, DMSO) δ 173.84, 169.93, 159.34, 153.06, 151.29, 147.68, 139.11, 138.88, 138.40, 137.63, 123.72, 117.38, 113.98, 113.86, 112.65, 111.70, 108.76, 56.30, 55.85, 34.53, 31.06, 30.99, 28.77, 8.76. ESI-TOF HRMS: m/z 510.2335 (C₂₆H₃₁N₅O₆+H⁺ requires 510.2347).

www. Preparation of D7

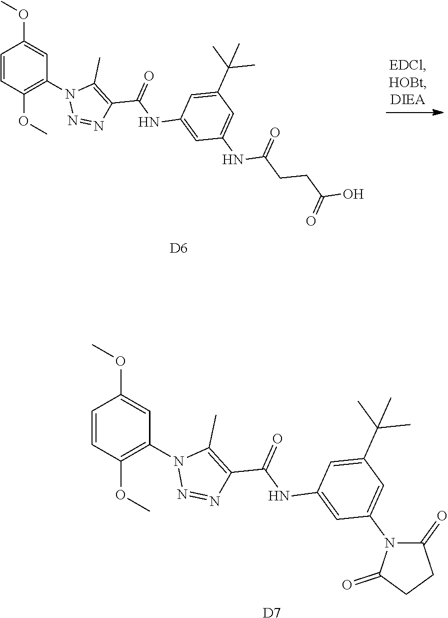 [SMILES: COc1ccc(OC)c(-n2nnc(C(=O)Nc3cc(NC(=O)CCC(=O)O)cc(C(C)(C)C)c3)c2C)c1 ; "D6" ; COc1ccc(OC)c(-n2nnc(C(=O)Nc3cc(N4C(=O)CCC4=O)cc(C(C)(C)C)c3)c2C)c1 ; "D7"]

To a solution of 4-((3-(tert-butyl)-5-(1-(2,5-dimethoxyphenyl)-5-methyl-1H-1,2,3-triazole-4-carboxamido)phenyl)amino)-4-oxobutanoic acid (0.102 g, 0.2 mmol) in DMF 5 mL was added HOBt, 80% (0.041 g, 0.240 mmol) and EDCI (0.058 g, 0.300 mmol), followed by N-ethyl-N-isopropylpropan-2-amine (0.052 g, 0.400 mmol). The suspension was stirred at room temperature for overnight. Then the reaction mixture was diluted with water (20 mL) and extracted with EtOAc (25 mL×2). The combine EtOAc layers were washed with water (20 mL), dried with anhydrous Na₂SO₄, and concentrated. The residue was purified by silica gel chromatography (0% to 100% EA in hexane) to give product as a white solid (74.1 mg, 76% yield). ¹H NMR (400 MHz, Chloroform-d) δ 9.15 (s, 1H), 7.73 (t, J=1.9 Hz, 1H), 7.61 (t, J=1.9 Hz, 1H), 7.09 (dd, J=9.1, 3.0 Hz, 1H), 7.05-7.00 (m, 2H), 6.95 (d, J=3.0 Hz, 1H), 3.82 (s, 3H), 3.76 (s, 3H), 2.89 (s, 4H), 2.50 (s, 3H), 1.34 (s, 9H). 13C NMR (101 MHz, CDCl3) δ 176.22, 159.33, 153.70, 153.28, 148.02, 139.60, 138.39, 137.73, 132.23, 124.27, 119.70, 117.58, 117.13, 115.26, 113.76, 113.34, 56.34, 56.02, 35.03, 31.20, 28.49, 9.22. ESI-TOF HRMS: m/z 492.2242 (C₂₆H₂₉N₅O₅+H⁺ requires 492.2242).

xxx. Preparation of D8

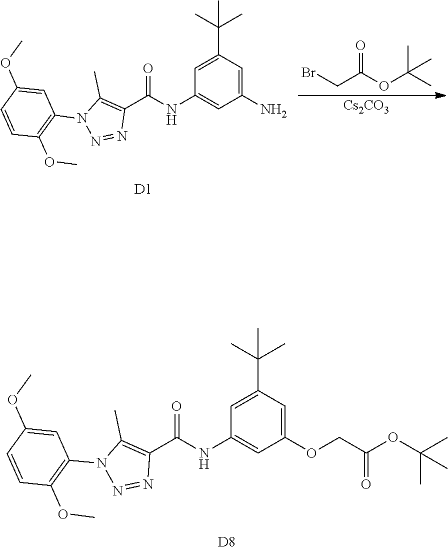

The preparation of compound D8 was prepared by reaction of N-(3-amino-5-(tert-butyl)phenyl)-1-(2,5-dimethoxyphenyl)-5-methyl-1H-1,2,3-triazole-4-carboxamide and tert-butyl 2-bromoacetate according to the procedure for B14 synthesis. 407.1 mg, 64% yield, white solid. ¹H NMR (500 MHz, DMSO-d6) δ 10.02 (s, 1H), 7.28 (d, J=9.2 Hz, 1H), 7.22 (dd, J=9.1, 3.1 Hz, 1H), 7.15 (d, J=3.0 Hz, 1H), 7.11 (d, J=1.7 Hz, 1H), 7.08 (t, J=2.0 Hz, 1H), 6.33 (t, J=1.9 Hz, 1H), 5.91 (t, J=6.5 Hz, 1H), 3.78 (s, 3H), 3.76-3.72 (m, 5H), 2.38 (s, 3H), 1.43 (s, 9H), 1.24 (s, 9H). ¹³C NMR (126 MHz, DMSO) δ 171.09, 159.63, 153.55, 151.88, 148.57, 148.18, 139.40, 139.19, 138.26, 124.23, 117.87, 114.48, 114.35, 106.83, 105.65, 102.17, 80.85, 56.79, 56.35, 46.13, 34.87, 31.62, 28.23, 9.24. ESI-TOF HRMS: m/z 524.2877 (C₂₈H₃₇N₅O₅+H⁺ requires 524.2867).

yyy. Preparation of D9

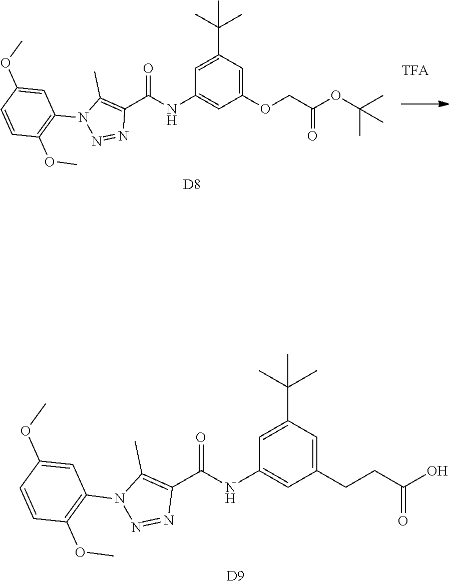 [SMILES: COc1ccc(OC)c(-n2nnc(C(=O)Nc3cc(OCC(=O)OC(C)(C)C)cc(C(C)(C)C)c3)c2C)c1 ; "D8" ; COc1ccc(OC)c(-n2nnc(C(=O)Nc3cc(CCC(=O)O)cc(C(C)(C)C)c3)c2C)c1 ; "D9"]

The preparation of compound D9 was prepared by reaction of tert-butyl (3-(tert-butyl)-5-(1-(2,5-dimethoxyphenyl)-5-methyl-1H-1,2,3-triazole-4-carboxamido)phenyl)glycinate with TFA according to the procedure for B25 synthesis. 124.1 mg, 69%, white solid. ¹H NMR (500 MHz, DMSO-d6) δ 10.03 (s, 1H), 7.28 (d, J=9.2 Hz, 1H), 7.22 (dd, J=9.1, 3.1 Hz, 1H), 7.16 (t, J=1.8 Hz, 1H), 7.14 (d, J=3.1 Hz, 1H), 7.00 (t, J=2.0 Hz, 1H), 6.39 (t, J=1.9 Hz, 1H), 3.78 (s, 3H), 3.77 (s, 2H), 3.75 (s, 3H), 2.37 (s, 3H), 1.24 (s, 9H). ¹³C NMR (126 MHz, DMSO) δ 172.09, 158.55, 152.46, 150.79, 147.50, 147.09, 138.36, 138.11, 137.17, 123.15, 116.78, 113.38, 113.27, 105.66, 105.12, 100.59, 55.71, 55.27, 44.18, 33.79, 30.56, 8.20. ESI-TOF HRMS: m/z 468.2239 (C₂₄H₂₉N₅O₅+H⁺ requires 468.2241).

zzz. Preparation of D10

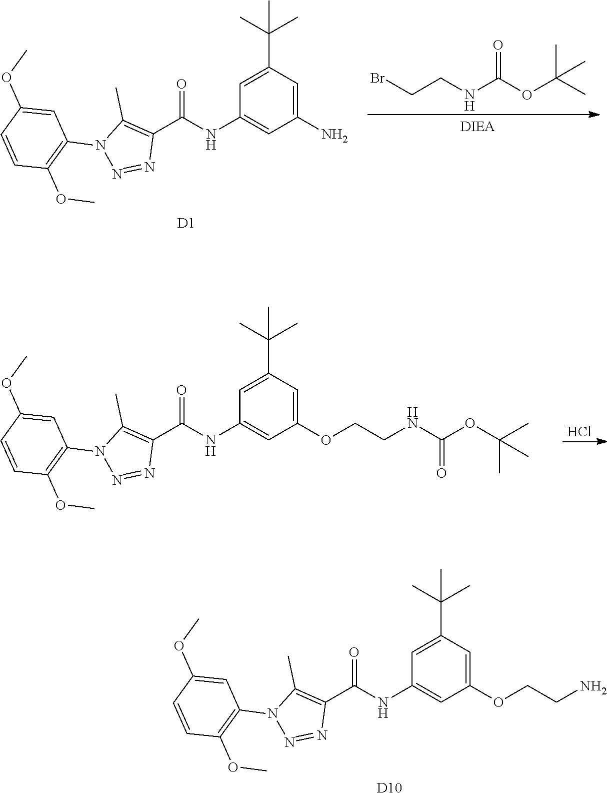

Tert-butyl (2-bromoethyl)carbamate (301 mg, 1.343 mmol) was added to a solution of N-(3-amino-5-(tert-butyl)phenyl)-1-(2,5-dimethoxyphenyl)-5-methyl-1H-1,2,3-triazole-4-carboxamide (500 mg, 1.221 mmol), N-ethyl-N-isopropylpropan-2-amine (316 mg, 2.442 mmol) in toluene 5 mL, then the mixture was stirred at 80° C. for 3 h. Then the reaction mixture was added EA (50 mL) and washed by water (30 mL). The organic phase was concentrated and the residue was used for next step without purified.

To a stirred solution of tert-butyl (2-((3-(tert-butyl)-5-(1-(2,5-dimethoxyphenyl)-5-methyl-1H-1,2,3-triazole-4-carboxamido)phenyl)amino)ethyl)carbamate in dry DCM (10 ml) was added TFA (5 mL) and stirred at room temperature for 20 hours. Then the mixture was concentrated, and the residue was added EA (100 mL) and 1 N Na₂CO₃ solution (50 mL). The separated organic phase was washed by water (50 mL), brine (50 mL) and dried by MgSO₄. The organic phases filtered and concentrated. The residue was purified by Colum chromatography (0% to 100% acetonitrile in water) to give product as light brown solid (312.8 mg, 76%, two steps). ¹H NMR (500 MHz, DMSO-d6) δ 10.07 (s, 1H), 7.81 (s, 3H), 7.29 (d, J=9.3 Hz, 1H), 7.23 (dd, J=9.2, 3.1 Hz, 1H), 7.17 (t, J=1.7 Hz, 1H), 7.13 (d, J=3.1 Hz, 1H), 7.11 (t, J=2.0 Hz, 1H), 6.40 (t, J=1.9 Hz, 1H), 5.72 (s, 1H), 3.78 (s, 3H), 3.75 (s, 3H), 3.29 (d, J=16.7 Hz, 3H), 2.99 (t, J=6.2 Hz, 2H), 2.38 (s, 3H), 1.25 (s, 9H). 13C NMR (126 MHz, DMSO) δ 158.62, 152.47, 151.03, 147.43, 147.08, 138.52, 138.17, 137.12, 123.12, 116.77, 113.41, 113.29, 105.96, 105.29, 100.86, 55.73, 55.28, 40.27, 37.55, 33.83, 30.56, 8.19. ESI-TOF HRMS: m/z 453.2618 (C₂₄H₃₂N₆O₃+H⁺ requires 453.2609).

aaaa. Preparation of D11

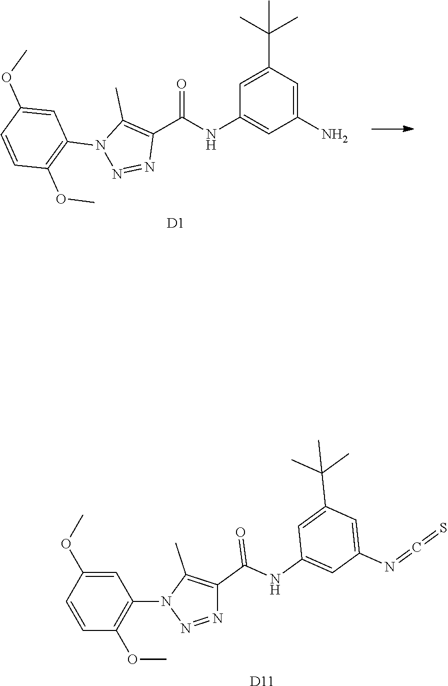 [SMILES: COc1ccc(OC)c(-n2nnc(C(=O)Nc3cc(N)cc(C(C)(C)C)c3)c2C)c1 ; "D1" ; COc1ccc(OC)c(-n2nnc(C(=O)Nc3cc(N=C=S)cc(C(C)(C)C)c3)c2C)c1 ; "D11"]

Thiophosgene (168 μl, 2.198 mmol) was added to a suspension solution of N-ethyl-N-isopropylpropan-2-amine (473 mg, 3.66 mmol) and N-(3-amino-5-(tert-butyl)phenyl)-1-(2,5-dimethoxyphenyl)-5-methyl-1H-1,2,3-triazole-4-carboxamide (750 mg, 1.832 mmol) in DCM (15 mL), then the mixture was stirred at room temperature for overnight. Then the reaction mixture was diluted with water (50 mL) and extracted with EtOAc (50 mL×2). The combine EtOAc layers were washed with water, dried with anhydrous Na₂SO₄, and concentrated. The residue was purified by silica gel chromatography (0% to 100% EA in hexane) to give product as a white solid (301 mg, 36%). ¹H NMR (500 MHz, DMSO-d6) δ 10.68 (s, 1H), 7.92 (t, J=1.8 Hz, 1H), 7.91 (t, J=1.9 Hz, 1H), 7.28 (d, J=9.2 Hz, 1H), 7.22 (dd, J=9.1, 3.1 Hz, 1H), 7.18 (t, J=1.8 Hz, 1H), 7.15 (d, J=3.1 Hz, 1H), 3.78 (s, 3H), 3.75 (s, 3H), 2.39 (s, 3H), 1.28 (s, 9H). ¹³C NMR (126 MHz, DMSO) δ 160.26, 153.68, 153.55, 148.16, 140.09, 139.85, 137.74, 133.52, 130.21, 124.11, 118.57, 117.93, 117.66, 114.95, 114.47, 114.35, 60.22, 56.81, 56.35, 35.31, 31.26, 9.32. ESI-TOF HRMS: m/z 452.1753 (C₂₃H₂₅N₅O₃S+H⁺ requires 452.1751).

bbbb. Preparation of D12 and D13

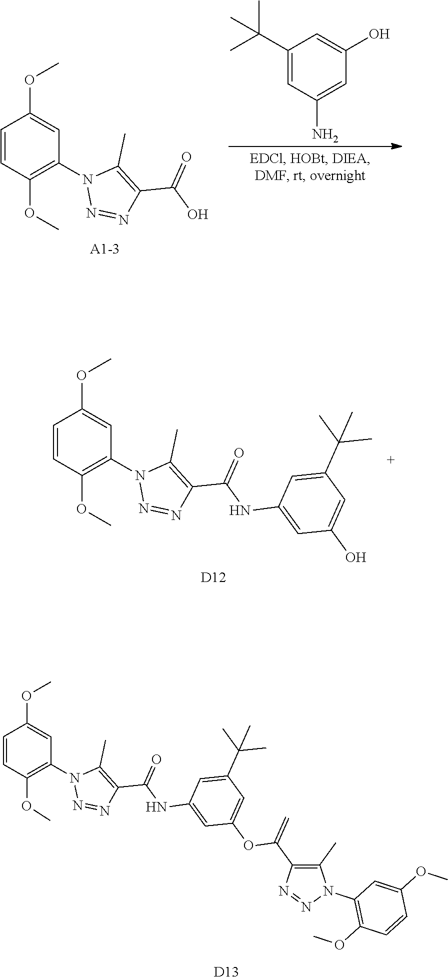 [SMILES: COc1ccc(OC)c(-n2nnc(C(=O)O)c2C)c1 ; CC(C)(C)c1cc(N)cc(O)c1 ; COc1ccc(OC)c(-n2nnc(C(=O)Nc3cc(O)cc(C(C)(C)C)c3)c2C)c1 ; "D12" ; C=C(Oc1cc(NC(=O)c2nnn(-c3cc(OC)ccc3OC)c2C)cc(C(C)(C)C)c1)c1nnn(-c2cc(OC)ccc2OC)c1C ; "D13"]

The preparation of compound D12 and D13 was prepared by reaction of 1-(2,5-dimethoxyphenyl)-5-methyl-1H-1,2,3-triazole-4-carboxylic acid and 3-amino-5-(tert-butyl)phenol according to the procedure for A1 synthesis. N-(3-(tert-butyl)-5-hydroxyphenyl)-1-(2,5-dimethoxyphenyl)-5-methyl-1H-1,2,3-triazole-4-carboxamide (892.4 mg, 55% yield) as a white solid. ¹H NMR (400 MHz, Chloroform-d) δ 9.16 (s, 1H), 7.57 (t, J=2.1 Hz, 1H), 7.09 (dd, J=9.1, 3.0 Hz, 1H), 7.06-6.98 (m, 2H), 6.95 (d, J=2.9 Hz, 1H), 6.71 (dd, J=2.3, 1.6 Hz, 1H), 3.81 (s, 3H), 3.76 (s, 3H), 2.51 (s, 3H), 1.29 (s, 9H). ¹³C NMR (101 MHz, CDCl3) δ 159.66, 156.61, 153.79, 153.69, 148.05, 139.72, 138.11, 137.83, 124.20, 117.62, 113.79, 113.35, 109.22, 109.06, 104.77, 56.34, 56.02, 34.79, 31.22, 9.29.

ESI-TOF HRMS: m/z 411.2022 (C₂₂H₂₆N₄O₄+H⁺ requires 411.2027).

3-(tert-butyl)-5-(1-(2,5-dimethoxyphenyl)-5-methyl-1H-1,2,3-triazole-4-carboxamido)phenyl 1-(2,5-dimethoxyphenyl)-5-methyl-1H-1,2,3-triazole-4-carboxylate (349.5 mg, 27% yield) as a white solid. ¹H NMR (400 MHz, Chloroform-d) δ 9.14 (s, 1H), 7.74 (t, J=2.0 Hz, 1H), 7.50 (t, J=1.8 Hz, 1H), 7.11-7.05 (m, 3H), 7.05-7.00 (m, 2H), 6.98 (dd, J=3.0, 1.3 Hz, 1H), 6.95 (dd, J=3.1, 1.3 Hz, 1H), 3.81 (s, 3H), 3.80 (s, 3H), 3.77 (d, J=1.6 Hz, 3H), 3.75 (d, J=1.6 Hz, 3H), 2.50 (s, 3H), 2.49 (s, 4H), 1.35 (s, 9H). ¹³C NMR (101 MHz, CDCl3) δ 160.28, 159.34, 153.73, 153.69, 153.66, 150.64, 148.01, 147.99, 142.05, 139.55, 138.37, 137.80, 135.38, 124.29, 124.27, 117.63, 117.53, 114.90, 114.37, 113.78, 113.42, 113.35, 110.75, 56.40, 56.34, 56.02, 56.01, 35.04, 31.23, 9.60, 9.22. ESI-TOF HRMS: m/z 656.2833 (C₃₄H₃₇N₇O₇+H^{|} requires 656.2827).

cccc. Preparation of D14

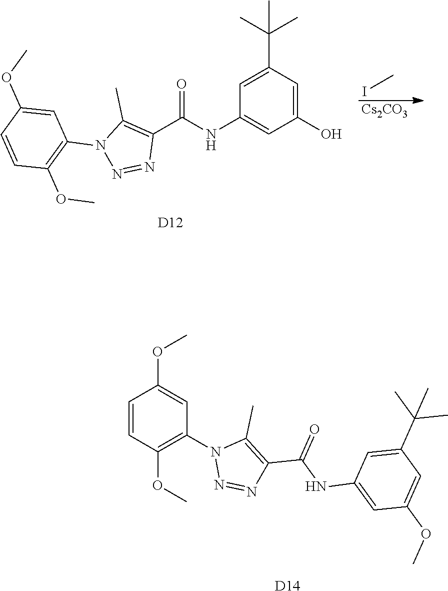

The preparation of compound D14 was prepared by reaction of N-(3-(tert-butyl)-5-hydroxyphenyl)-1-(2,5-dimethoxyphenyl)-5-methyl-1H-1,2,3-triazole-4-carboxamide and iodomethane according to the procedure for B4 synthesis. 118.2 mg, 70% yield, white solid. ¹H NMR (400 MHz, Chloroform-d) δ 9.09 (s, 1H), 7.41 (t, J=2.1 Hz, 1H), 7.16 (t, J=1.7 Hz, 1H), 7.09 (dd, J=9.1, 3.0 Hz, 1H), 7.03 (d, J=9.2 Hz, 1H), 6.96 (d, J=3.0 Hz, 1H), 6.75 (dd, J=2.4, 1.6 Hz, 1H), 3.85 (s, 3H), 3.82 (s, 3H), 3.76 (s, 3H), 2.52 (s, 3H), 1.33 (s, 9H). ¹³C NMR (101 MHz, CDCl3) δ 159.98, 159.37, 153.71, 153.49, 148.03, 139.41, 138.62, 137.96, 124.33, 117.50, 113.83, 113.34, 109.37, 108.59, 101.80, 56.34, 56.01, 55.36, 34.90, 31.27, 9.26. ESI-TOF HRMS: m/z 425.2173 (C₂₃H₂₈N₄O₄+H⁺ requires 425.2184).

dddd. Preparation of D15

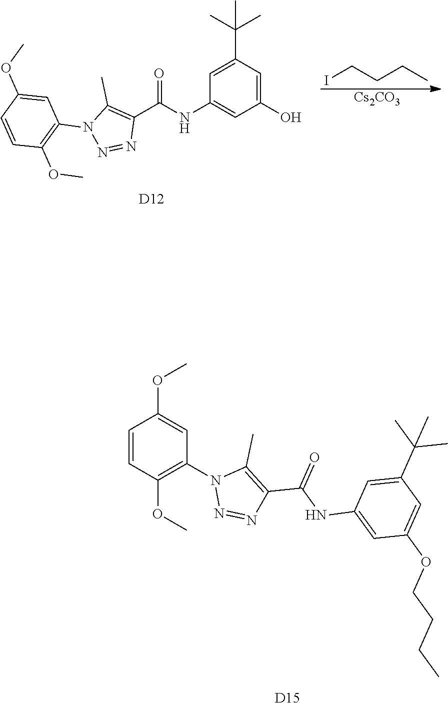

The preparation of compound D15 was prepared by reaction of N-(3-(tert-butyl)-5-hydroxyphenyl)-1-(2,5-dimethoxyphenyl)-5-methyl-1H-1,2,3-triazole-4-carboxamide and 1-iodobutane according to the procedure for B4 synthesis. 125.9 mg, 67% yield, white solid. ¹H NMR (400 MHz, Chloroform-d) δ 9.07 (s, 1H), 7.40 (t, J=2.1 Hz, 1H), 7.14 (t, J=1.8 Hz, 1H), 7.09 (dd, J=9.1, 3.0 Hz, 1H), 7.03 (d, J=9.1 Hz, 1H), 6.96 (d, J=2.9 Hz, 1H), 6.74 (dd, J=2.3, 1.6 Hz, 1H), 4.01 (t, J=6.5 Hz, 2H), 3.82 (s, 3H), 3.76 (s, 3H), 2.52 (s, 3H), 1.84-1.72 (m, 2H), 1.57-1.45 (m, 2H), 1.33 (s, 9H), 0.99 (t, J=7.4 Hz, 3H). ¹³C NMR (101 MHz, CDCl3) δ 159.57, 159.34, 153.71, 153.41, 148.04, 139.38, 138.53, 137.99, 124.35, 117.50, 113.83, 113.34, 109.18, 109.07, 102.38, 67.73, 56.34, 56.02, 34.89, 31.46, 31.28, 19.31, 13.92, 9.26. ESI-TOF HRMS: m/z 467.2658 (C₂₆H₃₄N₄O₄+H⁺ requires 467.2653).

eeee. Preparation of D16

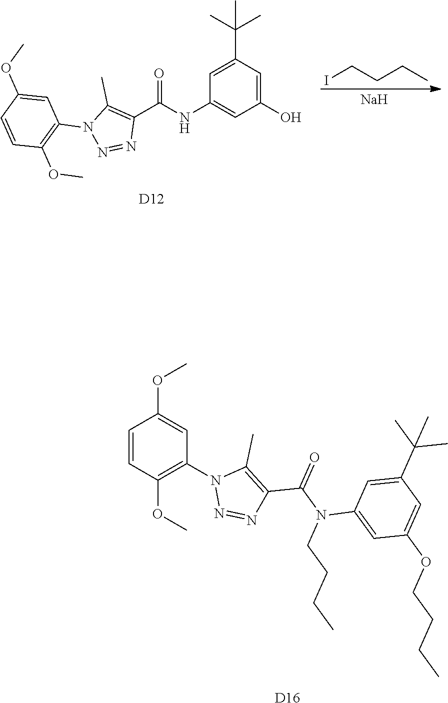

N-(3-(tert-butyl)-5-hydroxyphenyl)-1-(2,5-dimethoxyphenyl)-5-methyl-1H-1,2,3-triazole-4-carboxamide (100 mg, 0.244 mmol) was dissolved in 5 ml dry THF and stirred at 0° C. under N₂. Then sodium hydride (5.85 mg, 0.244 mmol) was added slowly and stirred for 0.5 hour. Then 1-iodobutane (44.8 mg, 0.244 mmol) was added and the reaction was stirred at room temperature under N₂ for 2 hours. Then the reaction solution was added water (50 mL), extracted by EtOAc (50 mL×2). The combine organic phases were dried over MgSO₄ and concentrated. The residue was purified by silica gel chromatography (0% to 100% EA in hexane) to give product as a white solid (81.2 mg, 64%). ¹H NMR (400 MHz, Chloroform-d) δ 6.98 (dd, J=9.0, 3.0 Hz, 1H), 6.92 (d, J=9.2 Hz, 1H), 6.76 (s, 2H), 6.65 (s, 1H), 6.55 (s, 1H), 4.01-3.91 (m, 3H), 3.86 (t, J=6.5 Hz, 2H), 3.72 (s, 3H), 3.62 (s, 3H), 2.11 (s, 3H), 1.77-1.60 (m, 4H), 1.52-1.32 (m, 4H), 1.19 (s, 9H), 1.01-0.85 (m, 6H). 13C NMR (101 MHz, CDCl3) δ 163.02, 159.23, 153.57, 152.90, 147.88, 143.41, 140.10, 136.28, 124.73, 117.65, 117.06, 113.71, 113.28, 110.85, 109.82, 67.70, 56.12, 55.87, 49.96, 34.68, 31.35, 31.10, 29.97, 20.14, 19.24, 13.86, 13.84, 9.03. ESI-TOF HRMS: m/z 523.3265 (C₃₀H₄₂N₄O₄+H⁺ requires 523.3279).

ffff. Preparation of D17

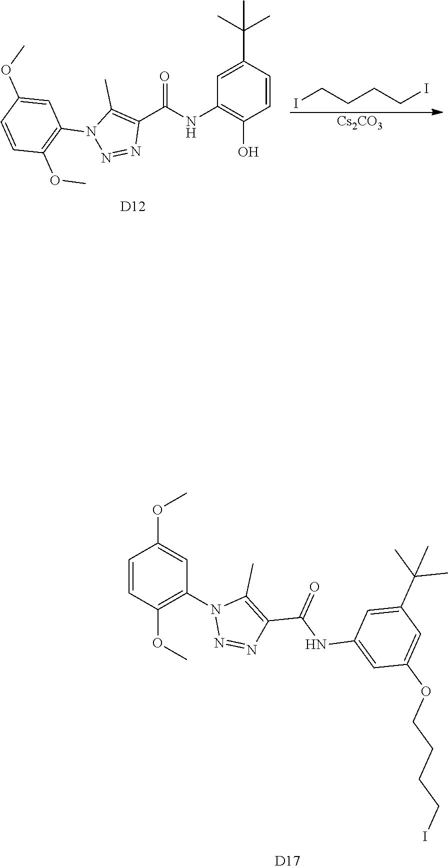

The preparation of compound D17 was prepared by reaction of N-(3-(tert-butyl)-5-hydroxyphenyl)-1-(2,5-dimethoxyphenyl)-5-methyl-1H-1,2,3-triazole-4-carboxamide and 1,4-diiodobutane according to the procedure for B4 synthesis. 122.6 mg, 69% yield, white solid. ¹H NMR (400 MHz, Chloroform-d) δ 9.08 (s, 1H), 7.43 (t, J=2.1 Hz, 1H), 7.12-7.07 (m, 2H), 7.03 (d, J=9.1 Hz, 1H), 6.96 (d, J=2.9 Hz, 1H), 6.73 (dd, J=2.3, 1.6 Hz, 1H), 4.04 (t, J=6.0 Hz, 2H), 3.82 (s, 3H), 3.76 (s, 3H), 3.28 (t, J=6.9 Hz, 2H), 2.52 (s, 3H), 2.11-2.02 (m, 2H), 1.96-1.87 (m, 2H), 1.33 (s, 9H). ¹³C NMR (101 MHz, CDCl3) δ 159.38, 159.28, 153.71, 153.50, 148.03, 139.41, 138.59, 137.94, 124.33, 117.50, 113.83, 113.34, 109.37, 108.88, 102.42, 66.64, 56.35, 56.02, 34.90, 31.27, 30.24, 9.26, 6.56.

ESI-TOF HRMS: m/z 593.1614 (C₂₆H₃₃IN₄O₄+H⁺ requires 593.1619).

gggg. Preparation of D18

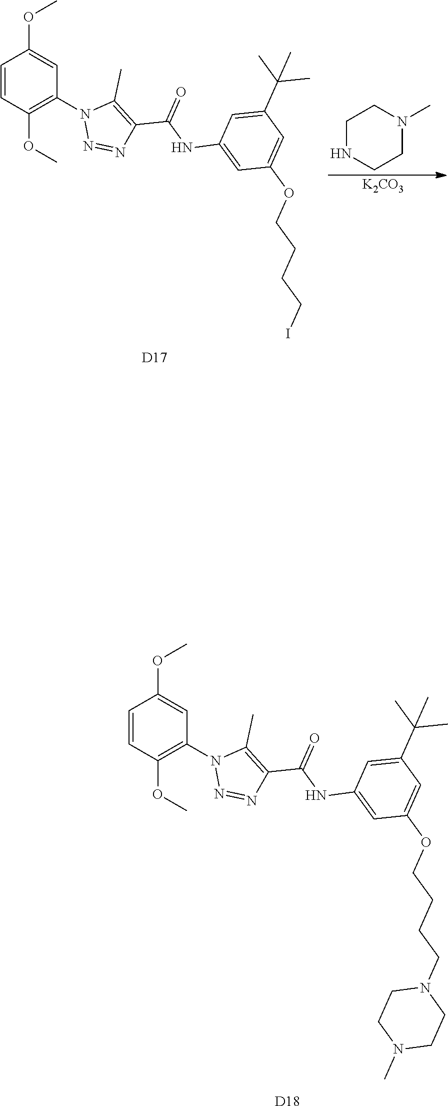

To a solution of N-(3-(tert-butyl)-5-(4-iodobutoxy)phenyl)-1-(2,5-dimethoxyphenyl)-5-methyl-1H-1,2,3-triazole-4-carboxamide (0.118 g, 0.2 mmol) in DMF 5 mL was added potassium carbonate (0.055 g, 0.400 mmol) and 1-methylpiperazine (0.040 g, 0.400 mmol). Then the reaction mixture was stirred at room temperature for overnight. The reaction mixture was diluted with water (20 mL) and extracted with EtOAc (25 mL×2). The combine EtOAc layers were washed with water, dried with anhydrous Na₂SO₄ and concentrated. The residue was purified by column chromatography (0% to 100% acetonitrile in water) to give product as a white solid (87.2 mg, 77% yield). ¹H NMR (400 MHz, Chloroform-d) δ 9.06 (s, 1H), 7.40 (t, J=2.1 Hz, 1H), 7.12 (t, J=1.7 Hz, 1H), 7.09 (dd, J=9.1, 3.0 Hz, 1H), 7.03 (d, J=9.1 Hz, 1H), 6.95 (d, J=3.0 Hz, 1H), 6.73 (dd, J=2.3, 1.6 Hz, 1H), 4.02 (t, J=6.3 Hz, 2H), 3.82 (s, 3H), 3.76 (s, 3H), 2.66-2.36 (m, 13H), 2.31 (s, 3H), 1.82 (dt, J=14.7, 6.4 Hz, 2H), 1.70 (ddd, J=15.0, 8.7, 5.7 Hz, 2H), 1.32 (s, 9H). ¹³C NMR (101 MHz, CDCl3) δ 159.43, 159.35, 153.71, 153.43, 148.03, 139.38, 138.53, 137.97, 124.33, 117.50, 113.82, 113.34, 109.24, 109.03, 102.39, 67.71, 58.21, 56.34, 56.02, 55.05, 53.01, 45.94, 34.89, 31.28, 27.41, 23.48, 9.25. ESI-TOF HRMS: m/z 565.3506 (C₃₁H₄₄N₆O₄+H⁺ requires 565.3497).

hhhh. Preparation of D19

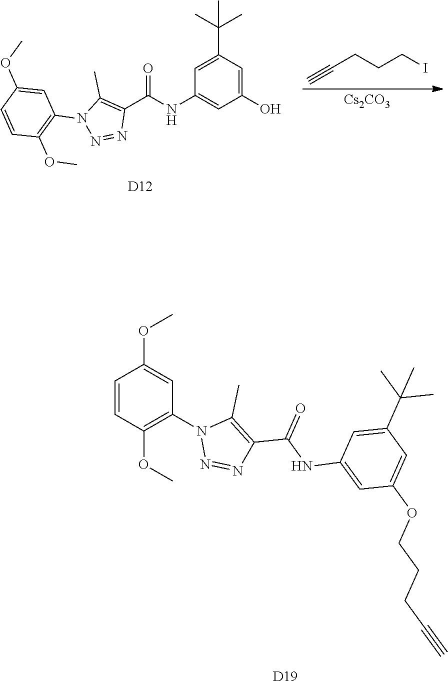

The preparation of compound D19 was prepared by reaction of N-(3-(tert-butyl)-5-hydroxyphenyl)-1-(2,5-dimethoxyphenyl)-5-methyl-1H-1,2,3-triazole-4-carboxamide and 5-iodopent-1-yne according to the procedure for B4 synthesis. 188.9 mg, 81%, white solid. ¹H NMR (400 MHz, Chloroform-d) δ 9.08 (s, 1H), 7.42 (t, J=2.1 Hz, 1H), 7.14 (t, J=1.8 Hz, 1H), 7.09 (dd, J=9.1, 3.0 Hz, 1H), 7.03 (d, J=9.1 Hz, 1H), 6.96 (d, J=2.9 Hz, 1H), 6.74 (dd, J=2.3, 1.6 Hz, 1H), 4.12 (t, J=6.1 Hz, 2H), 3.82 (s, 3H), 3.76 (s, 3H), 2.52 (s, 3H), 2.43 (td, J=7.1, 2.7 Hz, 2H), 2.06-2.00 (m, 2H), 1.64-1.61 (m, 1H), 1.33 (s, 9H). ¹³C NMR (101 MHz, CDCl3) δ 159.36, 159.28, 153.71, 153.49, 148.03, 139.41, 138.57, 137.96, 124.33, 117.51, 113.82, 113.34, 109.41, 108.91, 102.53, 83.64, 68.83, 66.18, 56.34, 56.02, 34.90, 31.27, 28.35, 15.26, 9.25. ESI-TOF HRMS: m/z 477.2495 (C₂₇H₃₂N₄O₄+H⁺ requires 477.2497).

iiii. Preparation of D20

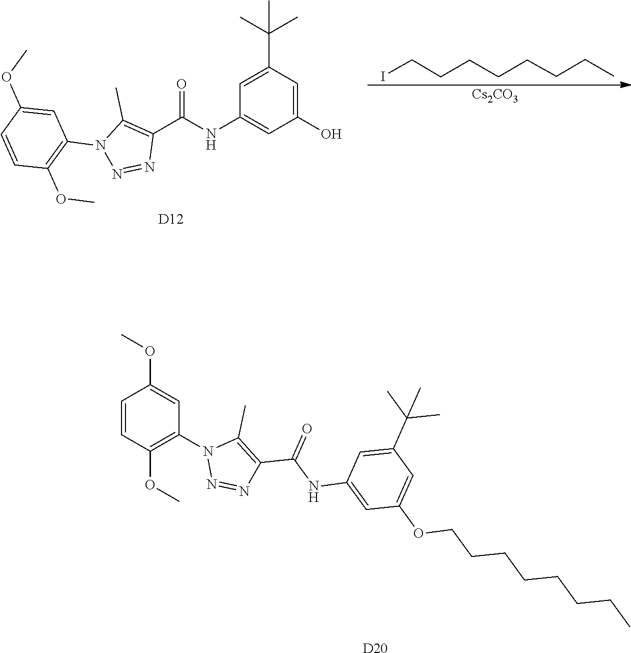

The preparation of compound D20 was prepared by reaction of N-(3-(tert-butyl)-5-hydroxyphenyl)-1-(2,5-dimethoxyphenyl)-5-methyl-1H-1,2,3-triazole-4-carboxamide and 1-iodooctane according to the procedure for B4 synthesis. 89.8 mg, 71% yield, colorless oil. ¹H NMR (400 MHz, Chloroform-d) δ 9.09 (s, 1H), 7.40 (t, J=2.1 Hz, 1H), 7.14 (t, J=1.7 Hz, 1H), 7.08 (dd, J=9.1, 3.0 Hz, 1H), 7.02 (d, J=9.2 Hz, 1H), 6.95 (d, J=3.0 Hz, 1H), 6.76-6.72 (m, 1H), 4.00 (t, J=6.5 Hz, 2H), 3.80 (s, 3H), 3.75 (s, 3H), 2.52 (s, 3H), 1.79 (p, J=6.7 Hz, 2H), 1.57-1.41 (m, 2H), 1.39-1.25 (m, 17H), 0.93-0.83 (m, 3H). ¹³C NMR (101 MHz, CDCl3) δ 159.56, 159.34, 153.70, 153.38, 148.01, 139.37, 138.55, 137.98, 124.33, 117.45, 113.84, 113.33, 109.17, 109.03, 102.42, 68.03, 56.31, 55.98, 34.88, 31.83, 31.27, 29.40, 29.38, 29.26, 26.11, 22.67, 14.11, 9.24. ESI-TOF HRMS: m/z 523.3282 (C₃₀H₄₂N₄O₄+H⁺ requires 523.3279).

jjjj. Preparation of D21 and D22

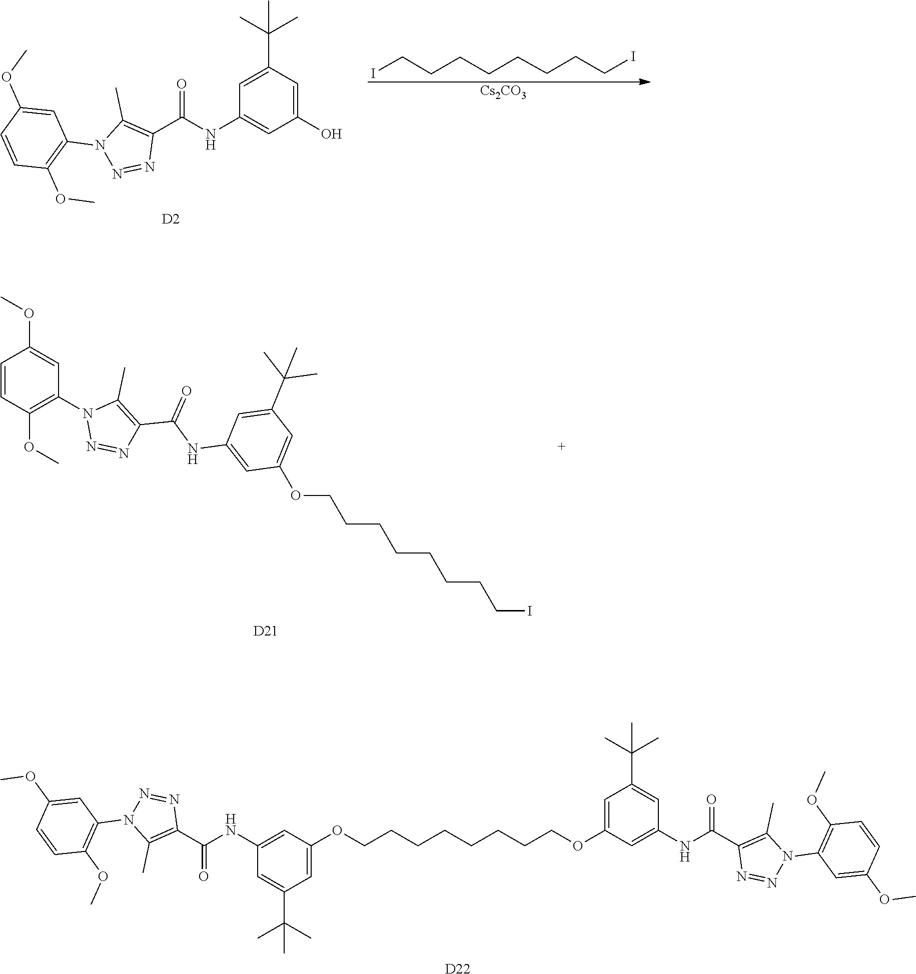

The preparation of compound D21 and D22 was prepared by reaction of N-(3-(tert-butyl)-5-hydroxyphenyl)-1-(2,5-dimethoxyphenyl)-5-methyl-1H-1,2,3-triazole-4-carboxamide and 1,8-diiodooctane according to the procedure for B4 synthesis.

N-(3-(tert-butyl)-5-((8-iodooctyl)oxy)phenyl)-1-(2,5-dimethoxyphenyl)-5-methyl-1H-1,2,3-triazole-4-carboxamide, 452.9 mg, 57% yield, colorless oil. ¹H NMR (400 MHz, Chloroform-d) δ 9.07 (s, 1H), 7.40 (t, J=2.1 Hz, 1H), 7.13 (t, J=1.7 Hz, 1H), 7.09 (dd, J=9.1, 3.0 Hz, 1H), 7.03 (d, J=9.1 Hz, 1H), 6.96 (d, J=2.9 Hz, 1H), 6.74 (dd, J=2.3, 1.6 Hz, 1H), 4.00 (t, J=6.5 Hz, 2H), 3.82 (s, 3H), 3.76 (s, 3H), 3.19 (t, J=7.0 Hz, 2H), 2.52 (s, 3H), 1.92-1.71 (m, 4H), 1.53-1.35 (m, 8H), 1.33 (s, 9H). ¹³C NMR (101 MHz, CDCl3) δ 159.52, 159.35, 153.71, 153.42, 148.03, 139.38, 138.54, 137.97, 124.34, 117.50, 113.83, 113.34, 109.20, 109.04, 102.39, 67.93, 56.35, 56.02, 34.89, 33.55, 31.28, 30.45, 29.32, 29.20, 28.48, 26.01, 9.26, 7.27. ESI-TOF HRMS: m/z 523.3282 (C₃₀H₄₂N₄O₄+H⁺ requires 523.3279).

N,N-((octane-1,8-diylbis(oxy))bis(5-(tert-butyl)-3,1-phenylene))bis(1-(2,5-dimethoxyphenyl)-5-methyl-1H-1,2,3-triazole-4-carboxamide), 143.8 mg, 12.7% yield, white solid. ¹H NMR (500 MHz, DMSO-d6) δ 10.31 (s, 2H), 7.52 (t, J=1.7 Hz, 2H), 7.46 (t, J=2.1 Hz, 2H), 7.28 (d, J=9.2 Hz, 2H), 7.22 (dd, J=9.2, 3.1 Hz, 2H), 7.14 (d, J=3.1 Hz, 2H), 6.65 (t, J=2.0 Hz, 2H), 3.96 (t, J=6.5 Hz, 4H), 3.78 (s, 6H), 3.75 (s, 6H), 2.38 (s, 6H), 1.74 (p, J=6.7 Hz, 4H), 1.42 (dt, J=31.4, 6.1 Hz, 8H), 1.27 (s, 18H). ¹³C NMR (126 MHz, DMSO) δ 158.83, 157.99, 152.48, 151.67, 147.09, 138.76, 138.37, 137.01, 123.12, 116.80, 113.39, 113.27, 109.18, 106.90, 102.72, 66.69, 55.71, 55.27, 33.99, 30.48, 28.21, 28.16, 24.97, 8.22. ESI-TOF HRMS: m/z 649.2252 (C₃₀H₄₁IN₄O₄+H⁺ requires 649.2245).

kkkk. Preparation of D23

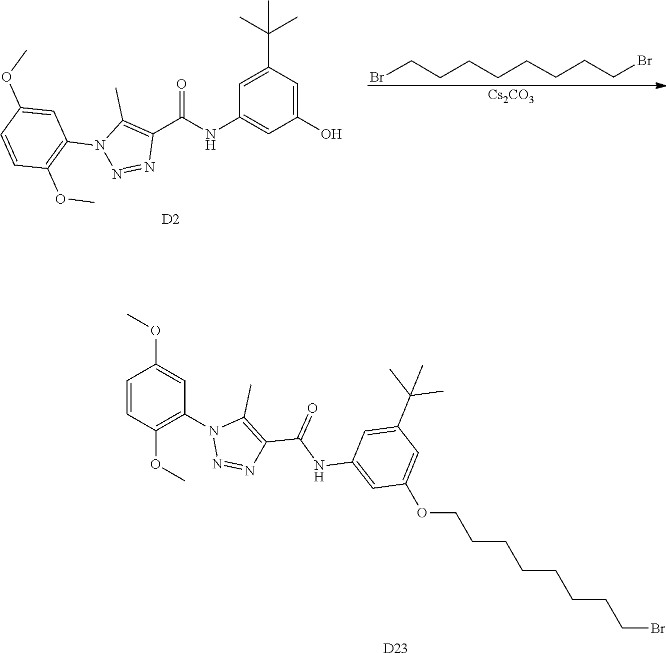

The preparation of compound D22 was prepared by reaction of N-(3-(tert-butyl)-5-hydroxyphenyl)-1-(2,5-dimethoxyphenyl)-5-methyl-1H-1,2,3-triazole-4-carboxamide and 1,8-dibromooctane according to the procedure for B4 synthesis. 628.3 mg, 86%, colorless oil. ¹H NMR (500 MHz, DMSO-d6) δ 10.31 (s, 1H), 7.51 (t, J=1.7 Hz, 1H), 7.44 (t, J=2.1 Hz, 1H), 7.28 (d, J=9.2 Hz, 1H), 7.22 (dd, J=9.2, 3.1 Hz, 1H), 7.14 (d, J=3.0 Hz, 1H), 6.64 (t, J=2.0 Hz, 1H), 3.94 (t, J=6.5 Hz, 2H), 3.78 (s, 3H), 3.75 (s, 3H), 3.53 (t, J=6.7 Hz, 2H), 2.38 (s, 3H), 1.84-1.76 (m, 2H), 1.75-1.68 (m, 2H), 1.46-1.30 (m, 8H), 1.27 (s, 9H). ¹³C NMR (126 MHz, DMSO) δ 158.83, 157.97, 152.47, 151.68, 147.09, 138.74, 138.37, 137.00, 123.10, 116.81, 113.39, 113.28, 109.18, 106.88, 102.71, 66.65, 55.72, 55.28, 34.61, 33.99, 31.63, 30.48, 28.08, 28.03, 27.45, 26.87, 24.86, 8.22. ESI-TOF HRMS: m/z 601.2390 (C₃₀H₄₁BrN₄O₄+H⁺ requires 601.2384).

llll. Preparation of D24

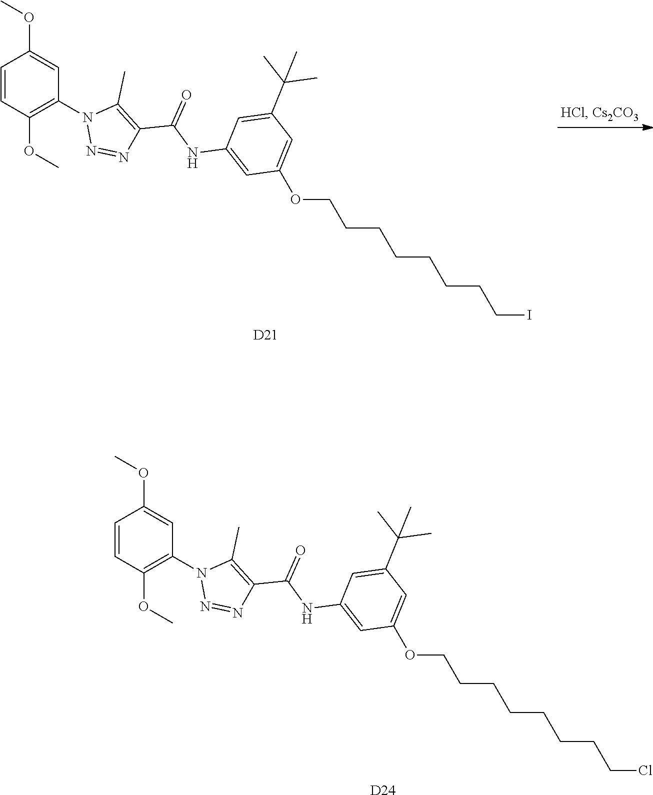 [SMILES: COc1ccc(OC)c(-n2nnc(C(=O)Nc3cc(OCCCCCCCCI)cc(C(C)(C)C)c3)c2C)c1 ; "D21" ; COc1ccc(OC)c(-n2nnc(C(=O)Nc3cc(OCCCCCCCCCl)cc(C(C)(C)C)c3)c2C)c1 ; "D24"]

To a solution of N-(3-(tert-butyl)-5-((8-iodooctyl)oxy)phenyl)-1-(2,5-dimethoxyphenyl)-5-methyl-1H-1,2,3-triazole-4-carboxamide (57 mg, 0.088 mmol) in DMF 5 mL was added hydrochloride (0.1 mmol) and Cs₂CO₃ (31.5 mg, 0.097 mmol). Then stirred at 80° C. for overnight. Then the reaction mixture was diluted with water (50 mL) and extracted with EtOAc (50 mL×2). The combine EtOAc layers were washed with water, dried with anhydrous Na2SO4 and concentrated. The residue was purified by silica gel chromatography (0% to 100% EA in hexane to give product (35.2 mg, 72% yield). ¹H NMR (400 MHz, Chloroform-d) δ 9.08 (s, 1H), 7.41 (t, J=2.1 Hz, 1H), 7.13 (t, J=1.8 Hz, 1H), 7.09 (dd, J=9.1, 3.0 Hz, 1H), 7.03 (d, J=9.1 Hz, 1H), 6.95 (d, J=2.9 Hz, 1H), 6.74 (dd, J=2.3, 1.6 Hz, 1H), 4.00 (t, J=6.5 Hz, 2H), 3.82 (s, 3H), 3.76 (s, 3H), 3.53 (t, J=6.8 Hz, 2H), 2.52 (s, 3H), 1.85-1.70 (m, 4H), 1.56-1.35 (m, 8H), 1.33 (s, 9H). ¹³C NMR (101 MHz, CDCl3) δ 159.52, 159.35, 153.71, 153.41, 148.02, 139.38, 138.55, 137.98, 124.34, 117.49, 113.83, 113.34, 109.20, 109.03, 102.39, 67.93, 56.34, 56.01, 45.16, 34.89, 32.64, 31.28, 29.32, 29.24, 28.83, 26.84, 26.01, 9.26. ESI-TOF HRMS: m/z 557.2899 (C₃₀H₄₁ClN₄O₄+H⁺ requires 557.2889).

mmmm. Preparation of D25

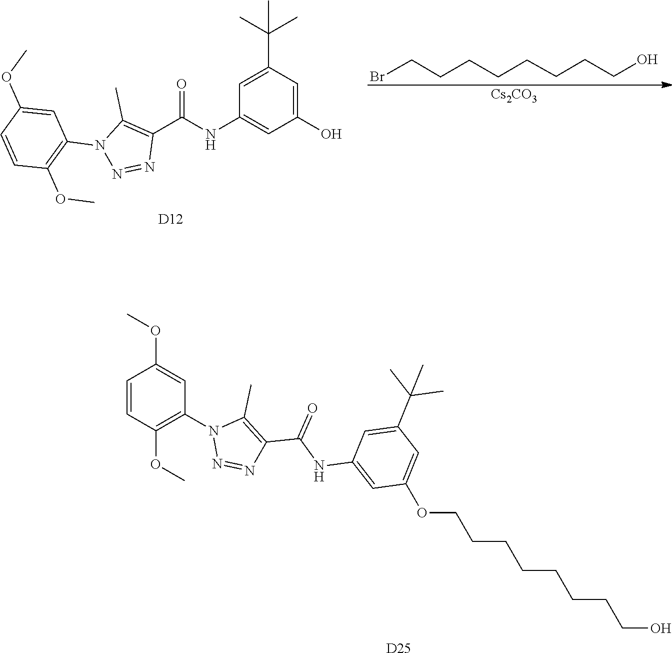

The preparation of compound D25 was prepared by reaction of N-(3-(tert-butyl)-5-hydroxyphenyl)-1-(2,5-dimethoxyphenyl)-5-methyl-1H-1,2,3-triazole-4-carboxamide and 8-bromooctan-1-ol according to the procedure for B4 synthesis. 496.5 mg, 76% yield, colorless oil. ¹H NMR (400 MHz, Chloroform-d) δ 9.09 (t, J=2.3 Hz, 1H), 7.40 (q, J=1.6, 1.1 Hz, 1H), 7.13 (t, J=1.8 Hz, 1H), 7.09 (dt, J=9.2, 2.6 Hz, 1H), 7.03 (dd, J=9.2, 1.8 Hz, 1H), 6.95 (dt, J=3.2, 1.6 Hz, 1H), 6.74 (dq, J=2.9, 1.5 Hz, 1H), 4.00 (td, J=6.5, 1.5 Hz, 2H), 3.82 (d, J=2.5 Hz, 3H), 3.76 (d, J=2.2 Hz, 3H), 3.68-3.61 (m, 2H), 2.52 (d, J=1.4 Hz, 3H), 1.85-1.72 (m, 2H), 1.63-1.53 (m, 2H), 1.52-1.45 (m, 2H), 1.43-1.34 (m, 6H), 1.32 (s, 9H). ¹³C NMR (101 MHz, CDCl₃) δ 159.53, 159.36, 153.70, 153.40, 148.02, 139.39, 138.53, 137.97, 124.32, 117.50, 113.82, 113.34, 109.21, 109.02, 102.47, 67.98, 63.04, 63.00, 56.33, 56.01, 34.89, 32.79, 31.28, 29.32, 26.01, 25.67, 9.26. ESI-TOF HRMS: m/z 539.3227(C₃₀H₄₂N₄O₅+H⁺ requires 539.3228).

nnnn. Preparation of D26

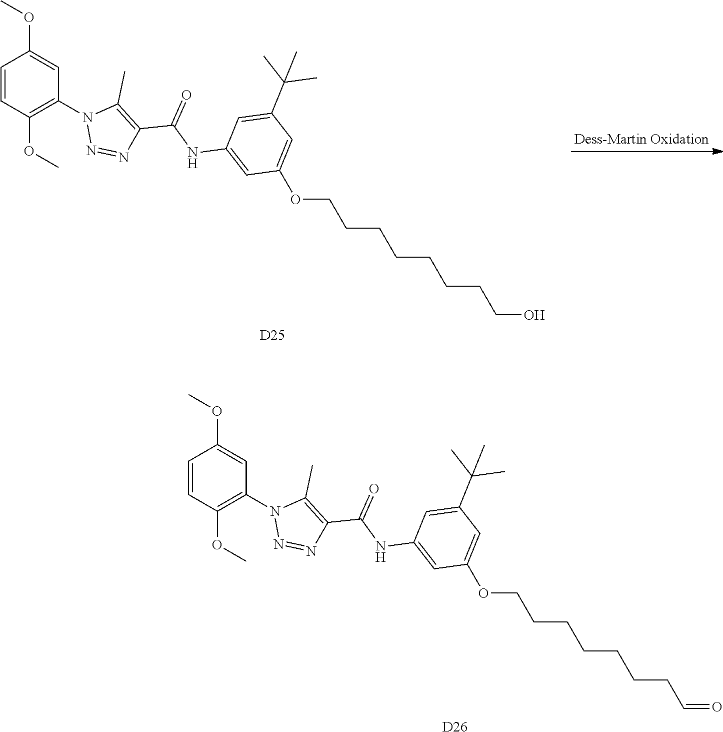 [SMILES: COc1ccc(OC)c(-n2nnc(C(=O)Nc3cc(OCCCCCCCCO)cc(C(C)(C)C)c3)c2C)c1 ; COc1ccc(OC)c(-n2nnc(C(=O)Nc3cc(OCCCCCCCC=O)cc(C(C)(C)C)c3)c2C)c1 ; "D25" ; "D26"]

To a solution of N-(3-(tert-butyl)-5-((8-hydroxyoctyl)oxy)phenyl)-1-(2,5-dimethoxyphenyl)-5-methyl-1H-1,2,3-triazole-4-carboxamide (250 mg, 0.464 mmol) in DCM (25 mL) was added Dess-Martin Oxidation (236 mg, 0.557 mmol). The reaction mixture was stirred at room temperature for overnight. Then the reaction mixture was then diluted with water (50 mL) and extracted with EtOAc (50 mL×2). The combine EtOAc layers were washed with water, dried with anhydrous Na₂SO₄, and concentrated. The residue was purified by silica gel chromatography (0% to 100% EA in hexane to give product (177.6 mg, 71% yield). ¹H NMR (400 MHz, Chloroform-d) δ 9.75 (t, J=1.8 Hz, 1H), 9.08 (s, 1H), 7.40 (t, J=2.1 Hz, 1H), 7.12 (t, J=1.7 Hz, 1H), 7.07 (dd, J=9.1, 3.0 Hz, 1H), 7.01 (d, J=9.1 Hz, 1H), 6.94 (d, J=3.0 Hz, 1H), 6.72 (dd, J=2.4, 1.6 Hz, 1H), 3.99 (t, J=6.4 Hz, 2H), 3.80 (s, 3H), 3.74 (s, 3H), 2.50 (s, 3H), 2.42 (td, J=7.3, 1.9 Hz, 2H), 1.84-1.72 (m, 2H), 1.64 (dq, J=12.3, 7.2 Hz, 2H), 1.53-1.43 (m, 2H), 1.43-1.34 (m, 4H), 1.31 (s, 9H). ¹³C NMR (101 MHz, CDCl3) δ 202.84, 159.49, 159.35, 153.69, 153.40, 148.00, 139.37, 138.55, 137.95, 124.30, 117.45, 113.83, 113.33, 109.21, 108.98, 102.41, 67.85, 56.31, 55.98, 43.86, 34.88, 31.26, 29.26, 29.12, 29.08, 25.90, 22.01, 9.23. ESI-TOF HRMS: m/z 537.3078 (C₃₀H₄₀N₄O₅+H⁺ requires 537.3072).

oooo. Preparation of D27

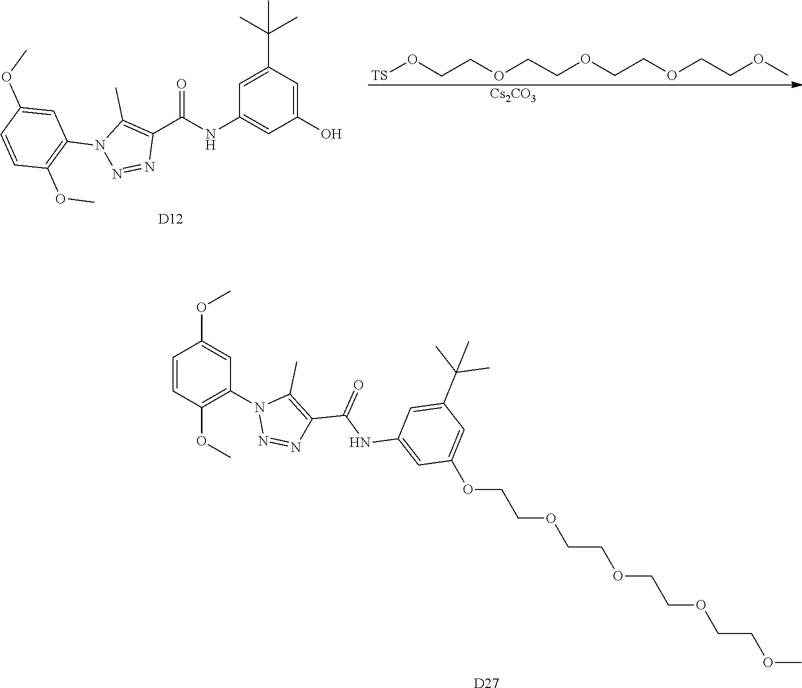

The preparation of compound D27 was prepared by reaction of N-(3-(tert-butyl)-5-hydroxyphenyl)-1-(2,5-dimethoxyphenyl)-5-methyl-1H-1,2,3-triazole-4-carboxamide and 2,5,8,11-tetraoxatridecan-13-yl 4-methylbenzenesulfonate according to the procedure for B4 synthesis. 53.8 mg, 65% yield, colorless oil. ¹H NMR (500 MHz, DMSO-d6) δ 10.33 (s, 1H), 7.53 (t, J=1.7 Hz, 1H), 7.45 (t, J=2.1 Hz, 1H), 7.29 (d, J=9.2 Hz, 1H), 7.22 (dd, J=9.1, 3.1 Hz, 1H), 7.15 (d, J=3.0 Hz, 1H), 6.68 (t, J=1.9 Hz, 1H), 4.09-4.06 (m, 2H), 3.78 (s, 3H), 3.75 (s, 5H), 3.60 (td, J=4.1, 1.1 Hz, 2H), 3.57-3.49 (m, 8H), 3.43-3.40 (m, 2H), 3.22 (s, 3H), 2.38 (s, 3H), 1.27 (s, 9H). ¹³C NMR (126 MHz, DMSO) δ 159.92, 158.82, 153.55, 152.83, 148.17, 139.84, 139.46, 138.07, 124.18, 117.89, 114.47, 114.35, 110.45, 107.94, 103.87, 71.74, 70.41, 70.30, 70.25, 70.04, 69.45, 67.43, 58.50, 56.80, 56.35, 35.11, 31.55, 9.30. ESI-TOF HRMS: m/z 601.3229 (C₃₁H₄₄N₄O₈+H⁺ requires 601.3232).

pppp. Preparation of D28

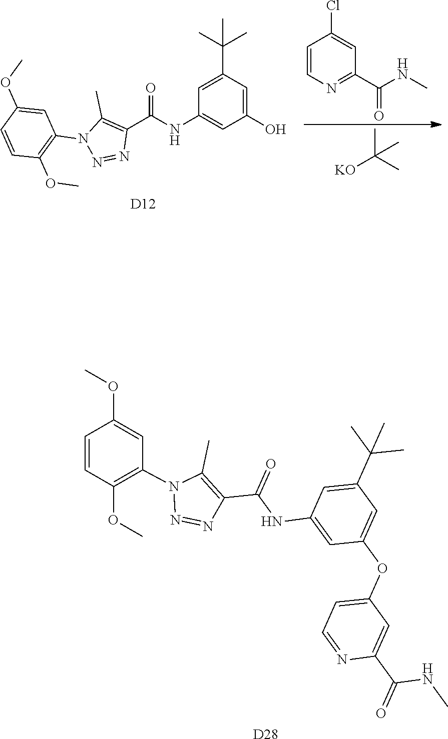

To a solution of N-(3-(tert-butyl)-5-hydroxyphenyl)-1-(2,5-dimethoxyphenyl)-5-methyl-1H-1,2,3-triazole-4-carboxamide (100 mg, 0.244 mmol) in N,N-dimethylacetamide (10 ml) was added potassium 2-methylpropan-2-olate (54.7 mg, 0.487 mmol). After the mixture was stirred at room temperature for 30 minutes, 4-chloro-N-methylpicolinamide (83 mg, 0.487 mmol) was added. The reaction mixture was stirred at 100° C. for overnight. The reaction solution was cooled to room temperature and diluted with water (100 ml), extracted with EA (3×40 ml) and the combine organic layers were dried over Na₂SO₄ and concentrated. The residue was purified by silica gel chromatography (0% to 100% EA in hexane to give product as a white solid (89.3 mg, 67% yield). ¹H NMR (400 MHz, Chloroform-d) δ 9.14 (s, 1H), 8.38 (d, J=5.6 Hz, 1H), 8.03 (d, J=7.5 Hz, 1H), 7.77 (d, J=2.5 Hz, 1H), 7.62 (t, J=2.0 Hz, 1H), 7.41 (t, J=1.8 Hz, 1H), 7.09 (dd, J=9.1, 3.0 Hz, 1H), 7.03 (d, J=9.1 Hz, 1H), 6.99 (dd, J=5.7, 2.5 Hz, 1H), 6.95 (d, J=3.0 Hz, 1H), 6.91 (t, J=1.9 Hz, 1H), 3.82 (s, 3H), 3.76 (s, 3H), 3.01 (d, J=5.1 Hz, 3H), 2.49 (s, 3H), 1.35 (s, 9H). ¹³C NMR (101 MHz, CDCl3) δ 166.64, 164.22, 159.41, 154.82, 153.96, 153.71, 151.98, 149.23, 148.00, 139.64, 139.22, 137.67, 124.25, 117.56, 114.11, 114.07, 113.78, 113.77, 113.34, 110.77, 109.47, 56.34, 56.02, 35.10, 31.20, 26.18, 9.21. ESI-TOF HRMS: m/z 545.2504 (C₂₉H₃₂N₆O₅+H⁺ requires 545.2507).

qqqq. Preparation of D29

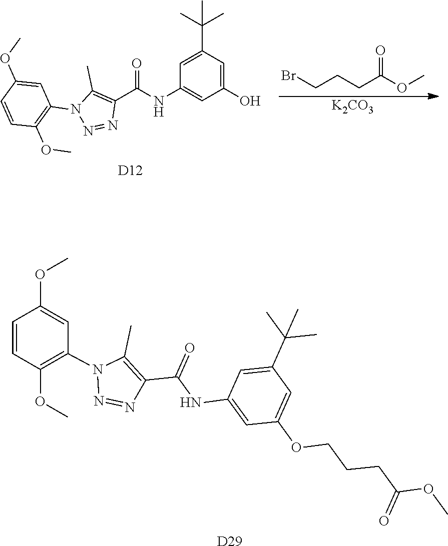

To a solution of N-(3-(tert-butyl)-5-hydroxyphenyl)-1-(2,5-dimethoxyphenyl)-5-methyl-1H-1,2,3-triazole-4-carboxamide (500 mg, 1.218 mmol) in DMF 20 mL was added methyl 4-bromobutanoate (221 mg, 1.218 mmol) and K₂CO₃ (337 mg, 2.436 mmol). The mixture was stirred at 80° C. for overnight. The reaction mixture was cool down and then diluted with water (50 mL) and extracted with EtOAc (50 mL×2). The combine EtOAc layers were washed with water, dried with anhydrous Na₂SO₄ and concentrated. The residue was purified by silica gel chromatography (0% to 100% EA in hexane) to give product as a white solid (489.1 mg, 79% yield). ¹H NMR (400 MHz, Chloroform-d) δ 9.07 (s, 1H), 7.41 (t, J=2.1 Hz, 1H), 7.12 (t, J=1.7 Hz, 1H), 7.09 (dd, J=9.1, 3.0 Hz, 1H), 7.03 (d, J=9.1 Hz, 1H), 6.96 (d, J=3.0 Hz, 1H), 6.72 (dd, J=2.3, 1.6 Hz, 1H), 4.05 (t, J=6.1 Hz, 2H), 3.82 (s, 3H), 3.76 (s, 3H), 3.70 (s, 3H), 2.55 (t, J=7.4 Hz, 2H), 2.52 (s, 3H), 2.17-2.09 (m, 2H), 1.32 (s, 9H). ¹³C NMR (101 MHz, CDCl3) δ 173.72, 159.34, 159.23, 153.71, 153.49, 148.03, 139.41, 138.57, 137.94, 124.33, 117.51, 113.82, 113.34, 109.40, 108.84, 102.50, 66.72, 56.35, 56.02, 51.63, 34.90, 31.27, 30.66, 24.74, 9.25. ESI-TOF HRMS: m/z 511.2561 (C₂₇H₃₄N₄O₆+H⁺ requires 511.2551).

rrrr. Preparation D30

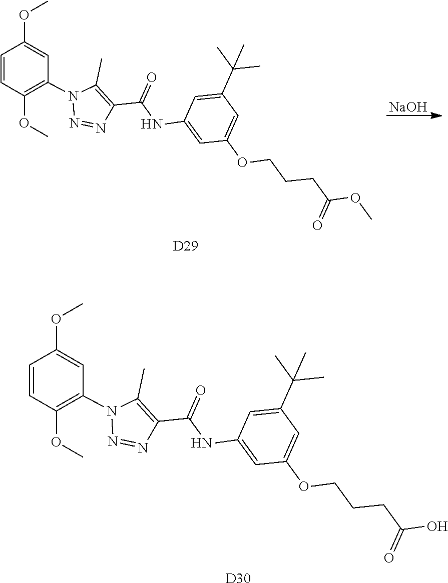 [SMILES: COC(=O)CCCOc1cc(NC(=O)c2nnn(-c3cc(OC)ccc3OC)c2C)cc(C(C)(C)C)c1 ; "NaOH" ; COc1ccc(OC)c(-n2nnc(C(=O)Nc3cc(OCCCC(=O)O)cc(C(C)(C)C)c3)c2C)c1 ; "D29" ; "D30"]

4-(3-(tert-butyl)-5-(1-(2,5-dimethoxyphenyl)-5-methyl-1H-1,2,3-triazole-4-carboxamido)phenoxy) butanoate (529 mg, 1.036 mmol), sodium hydroxide (414 mg, 10.36 mmol) was added to acetone (10 mL) and water (10 mL). The mixture was stirred at room temperature for overnight. The reaction mixture was then diluted with water (50 mL) and extracted with EtOAc (50 mL×2). The combine EtOAc layers were washed with water, dried with anhydrous Na₂SO₄ and concentrated. The residue was purified by silica gel chromatography (0% to 100% EA in hexane) to give product as a white solid (447.5 mg, 87% yield). ¹H NMR (400 MHz, Chloroform-d) δ 9.18 (s, 1H), 7.42 (t, J=2.1 Hz, 1H), 7.17 (t, J=1.7 Hz, 1H), 7.09 (dd, J=9.1, 3.0 Hz, 1H), 7.02 (d, J=9.1 Hz, 1H), 6.95 (d, J=3.0 Hz, 1H), 6.75-6.71 (m, 1H), 4.08 (t, J=6.0 Hz, 2H), 3.81 (s, 3H), 3.75 (s, 3H), 2.62 (t, J=7.3 Hz, 2H), 2.52 (s, 3H), 2.20-2.08 (m, 2H), 1.32 (s, 9H). ¹³C NMR (101 MHz, CDCl3) δ 178.69, 159.35, 159.14, 153.69, 153.47, 148.02, 139.56, 138.58, 137.93, 124.27, 117.56, 113.80, 113.34, 109.60, 108.92, 102.59, 66.58, 56.33, 56.01, 34.90, 31.26, 30.56, 24.48, 9.28.

ESI-TOF HRMS: m/z 497.2395 (C₂₆H₃₂N₄O₆+H⁺ requires 497.2395).

ssss. Preparation of D31

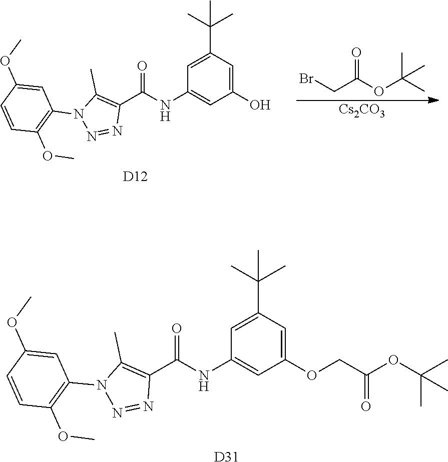

The preparation of compound D31 was prepared by reaction of N-(3-(tert-butyl)-5-hydroxyphenyl)-1-(2,5-dimethoxyphenyl)-5-methyl-1H-1,2,3-triazole-4-carboxamide and tert-butyl 2-bromoacetate according to the procedure for B4 synthesis. 571.1 mg, 89% yield, white solid. ¹H NMR (500 MHz, DMSO-d6) δ 10.36 (s, 1H), 7.54 (t, J=1.7 Hz, 1H), 7.47 (t, J=2.1 Hz, 1H), 7.28 (d, J=9.2 Hz, 1H), 7.22 (dd, J=9.1, 3.1 Hz, 1H), 7.15 (d, J=3.0 Hz, 1H), 6.63 (dd, J=2.4, 1.6 Hz, 1H), 4.62 (s, 2H), 3.78 (s, 3H), 3.75 (s, 3H), 2.38 (s, 3H), 1.45 (s, 9H), 1.27 (s, 9H). ¹³C NMR (126 MHz, DMSO) δ 167.35, 158.86, 156.96, 152.47, 151.71, 147.10, 138.81, 138.41, 136.97, 123.10, 116.82, 113.40, 113.28, 109.80, 106.29, 103.12, 80.73, 64.40, 55.72, 55.28, 34.04, 30.42, 27.11, 8.20. ESI-TOF HRMS: m/z 525.2718 (C₂₈H₃₆N₄O₆+H⁺ requires 525.2708).

tttt. Preparation of D32

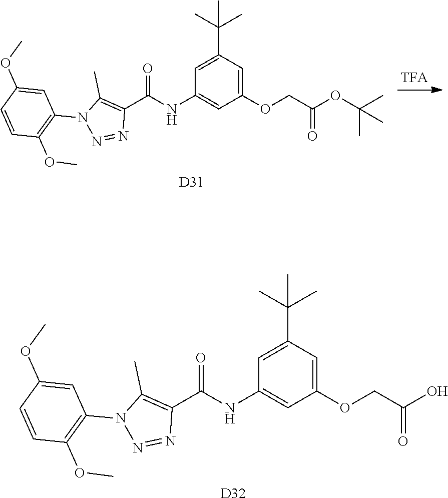 [SMILES: COc1ccc(OC)c(-n2nnc(C(=O)Nc3cc(OCC(=O)OC(C)(C)C)cc(C(C)(C)C)c3)c2C)c1 ; COc1ccc(OC)c(-n2nnc(C(=O)Nc3cc(OCC(=O)O)cc(C(C)(C)C)c3)c2C)c1 ; "D31" ; "D32"]

The preparation of compound D32 was prepared by reaction of tert-butyl 2-(3-(tert-butyl)-5-(1-(2,5-dimethoxyphenyl)-5-methyl-1H-1,2,3-triazole-4-carboxamido)phenoxy)acetate and TFA according to the procedure for B25 synthesis. 388.5 mg, 87% yield, white solid. ¹H NMR (500 MHz, DMSO-d6) δ 12.99 (s, 1H), 10.37 (s, 1H), 7.58 (t, J=1.7 Hz, 1H), 7.42 (t, J=2.1 Hz, 1H), 7.28 (d, J=9.2 Hz, 1H), 7.22 (dd, J=9.1, 3.1 Hz, 1H), 7.15 (d, J=3.1 Hz, 1H), 6.66 (t, J=2.1 Hz, 1H), 4.64 (s, 2H), 3.78 (s, 3H), 3.75 (s, 3H), 2.38 (s, 3H), 1.27 (s, 9H). ¹³C NMR (126 MHz, DMSO) δ 169.61, 158.86, 157.03, 152.47, 151.75, 147.09, 138.80, 138.41, 136.98, 123.10, 116.82, 113.39, 113.28, 109.72, 106.73, 102.72, 63.82, 55.72, 55.28, 34.03, 30.44, 8.24. ESI-TOF HRMS: m/z 469.2087 (C₂₄H₂₈N₄O₆+H⁺ requires 469.2082).

uuuu. Preparation of D33

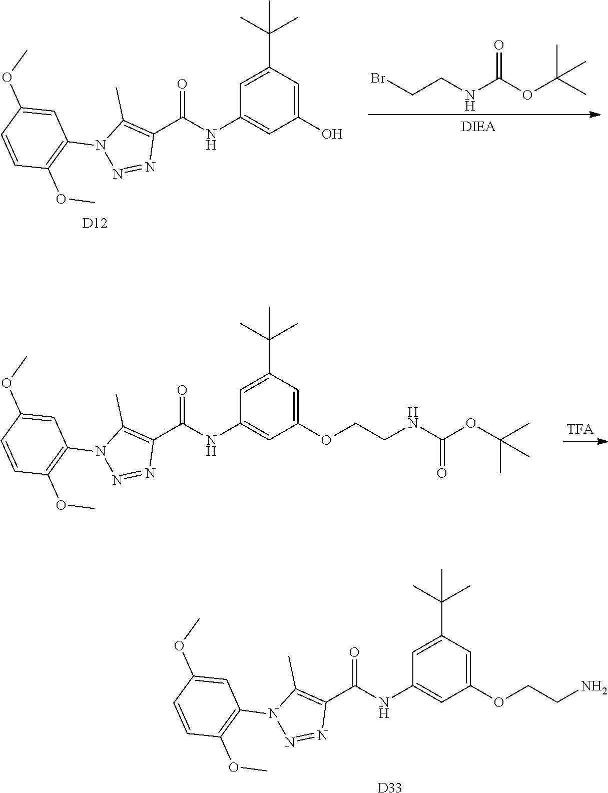

Preparation of D33 was prepared by reaction of tert-butyl 2-(3-(tert-butyl)-5-(1-(2,5-dimethoxyphenyl)-5-methyl-1H-1,2,3-triazole-4-carboxamido)phenoxy)acetate and TFA according to the procedure for B10 synthesis. 372.4 mg, 81% yield, white solid. ¹H NMR (400 MHz, DMSO-d6) δ 10.21 (s, 1H), 7.50 (t, J=1.7 Hz, 1H), 7.46 (t, J=2.1 Hz, 1H), 7.29 (d, J=9.2 Hz, 1H), 7.22 (dd, J=9.1, 3.0 Hz, 1H), 7.13 (d, J=3.0 Hz, 1H), 6.68 (dd, J=2.4, 1.6 Hz, 1H), 3.94 (t, J=5.8 Hz, 2H), 3.79 (s, 3H), 3.76 (s, 3H), 2.92 (t, J=5.7 Hz, 2H), 2.39 (s, 3H), 1.29 (s, 9H). ¹³C NMR (101 MHz, DMSO) δ 159.38, 158.56, 153.15, 152.31, 147.75, 139.26, 138.84, 137.58, 123.84, 117.41, 114.02 (2C), 109.94, 107.58, 103.54, 69.82, 56.38, 55.89, 40.84, 34.52, 31.01, 8.70. ESI-TOF HRMS: m/z 454.2449 (C₂₄H₃₂N₆O₃+H⁺ requires 454.2449).

vvvv. Preparation of D34

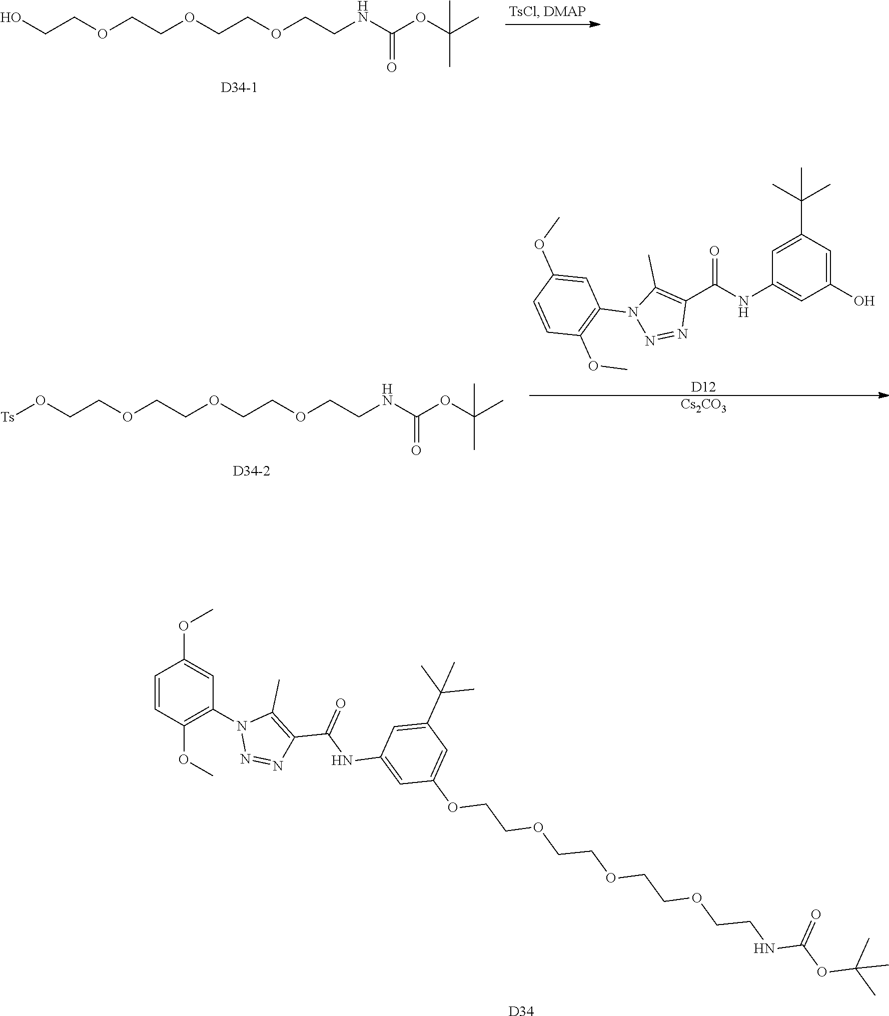

To a solution of tert-butyl (2-(2-(2-(2-hydroxyethoxy)ethoxy)ethoxy)ethyl)carbamate (0.7 g, 2.386 mmol) in pyridine 5 mL was added 4-methylbenzenesulfonyl chloride (0.500 g, 2.62 mmol) and N,N-dimethylpyridin-4-amine (0.292 g, 2.386 mmol). The suspension was stirred at room temperature for overnight. The reaction mixture was then diluted with water (50 mL) and extracted with EtOAc (50 mL×2). The combine EtOAc layers were washed with water, dried with anhydrous Na₂SO₄, and concentrated. The residue was purified by silica gel chromatography (0% to 100% EA in hexane) to give product (782.3 mg, 73% yield). ¹H NMR (400 MHz, Chloroform-d) δ 7.78 (dq, J=8.5, 2.1 Hz, 2H), 7.34-7.30 (m, 2H), 4.19-4.11 (m, 2H), 3.70-3.66 (m, 2H), 3.60-3.55 (m, 8H), 3.51 (dd, J=5.6, 4.9 Hz, 2H), 3.28 (q, J=5.2 Hz, 2H), 2.43 (s, 3H), 1.43 (s, 9H). ¹³C NMR (101 MHz, CDCl3) δ 155.93, 144.67, 133.35, 129.75, 127.91, 79.12, 70.78, 70.57, 70.52, 70.26, 70.19, 69.14, 68.72, 40.52, 28.39, 21.50.

A mixture of N-(3-(tert-butyl)-5-hydroxyphenyl)-1-(2,5-dimethoxyphenyl)-5-methyl-1H-1,2,3-triazole-4-carboxamide (0.5 g, 1.218 mmol), Cs₂CO₃ (0.397 g, 1.218 mmol) and 2,2-dimethyl-4-oxo-3,8,11,14-tetraoxA5-azahexadecan-16-yl 4-methylbenzenesulfonate (0.545 g, 1.218 mmol) was stirred in acetone 30 mL at room temperature for overnight. Then the reaction was stirred at 80° C. for another overnight. The reaction mixture was diluted with water (50 mL) and extracted with EtOAc (50 mL×2). The combine EtOAc layers were washed with water, dried with anhydrous Na₂SO₄, and concentrated. The residue was purified by silica gel chromatography (0% to 100% acetonitrile in water) to give product as a colorless oil, 0.73 g 87% yield. ¹H NMR (400 MHz, DMSO-d6) δ 10.20 (s, 1H), 7.51 (t, J=1.7 Hz, 1H), 7.44 (t, J=2.1 Hz, 1H), 7.28 (d, J=9.2 Hz, 1H), 7.22 (dd, J=9.1, 3.0 Hz, 1H), 7.12 (d, J=3.0 Hz, 1H), 6.68 (dd, J=2.3, 1.7 Hz, 1H), 6.59 (s, 1H), 4.11-4.07 (m, 2H), 3.79 (s, 3H), 3.78-3.76 (m, 2H), 3.76 (s, 3H), 3.62 (ddd, J=5.9, 3.8, 1.1 Hz, 2H), 3.58-3.48 (m, 6H), 3.39 (t, J=6.1 Hz, 2H), 2.39 (s, 3H), 1.37 (s, 9H), 1.29 (s, 9H). ¹³C NMR (101 MHz, DMSO) δ 159.38, 158.37, 155.49, 153.15, 152.37, 147.76, 139.26, 138.85, 137.58, 123.85, 117.42, 114.01, 110.04, 107.52, 103.60, 77.51, 69.93, 69.80, 69.74, 69.49, 69.17, 68.98, 67.07, 56.38, 55.89, 34.54, 31.01, 28.15, 13.99, 8.70. ESI-TOF HRMS: m/z 686.3766 (C₃₅H₅₁N₅O₉+H⁺ requires 686.3760).

wwww. Preparation of D35

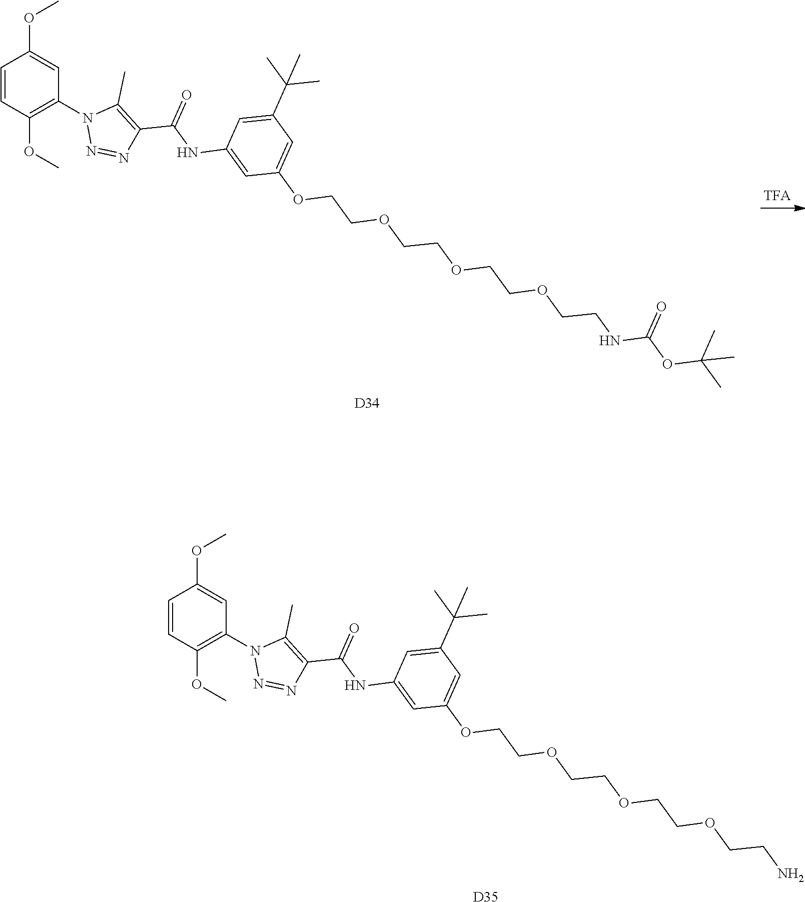 [SMILES: COc1ccc(OC)c(-n2nnc(C(=O)Nc3cc(OCCOCCOCCOCCNC(=O)OC(C)(C)C)cc(C(C)(C)C)c3)c2C)c1 ; COc1ccc(OC)c(-n2nnc(C(=O)Nc3cc(OCCOCCOCCOCCN)cc(C(C)(C)C)c3)c2C)c1 ; "D34" ; "D35"]

The preparation of compound D35 was prepared by reaction of tert-butyl (2-(2-(2-(2-(3-(tert-butyl)-5-(1-(2,5-dimethoxyphenyl)-5-methyl-1H-1,2,3-triazole-4-carboxamido)phenoxy)ethoxy)ethoxy)ethoxy)ethyl) carbamate and TFA according to the procedure for B25 synthesis. 76.5 mg, 90% yield, light brown oil. ¹H NMR (400 MHz, Chloroform-d) δ 9.04 (s, 1H), 7.39 (t, J=2.1 Hz, 1H), 7.16 (t, J=1.8 Hz, 1H), 7.08 (dd, J=9.1, 3.0 Hz, 1H), 7.02 (d, J=9.1 Hz, 1H), 6.94 (d, J=2.9 Hz, 1H), 6.77 (dd, J=2.4, 1.6 Hz, 1H), 4.17 (dd, J=5.8, 4.2 Hz, 2H), 3.86 (dd, J=5.5, 4.3 Hz, 2H), 3.81 (s, 3H), 3.75 (s, 3H), 3.74-3.71 (m, 2H), 3.70-3.62 (m, 8H), 3.55-3.51 (m, 2H), 3.36 (s, 3H), 2.51 (s, 3H), 1.32 (s, 9H). ¹³C NMR (101 MHz, CDCl3) δ 159.32, 159.25, 153.86, 153.46, 148.15, 139.29, 138.56, 137.99, 124.60, 117.49, 113.98, 113.57, 109.60, 109.15, 102.88, 71.98, 70.84, 70.67, 70.64, 70.51, 69.81, 67.60, 58.89, 56.42, 56.00, 34.86, 31.23, 9.12. ESI-TOF HRMS: m/z 586.3245 (C₃₀H₄₃N₅O₇+H⁺ requires 586.3235).

xxxx. Preparation of D36

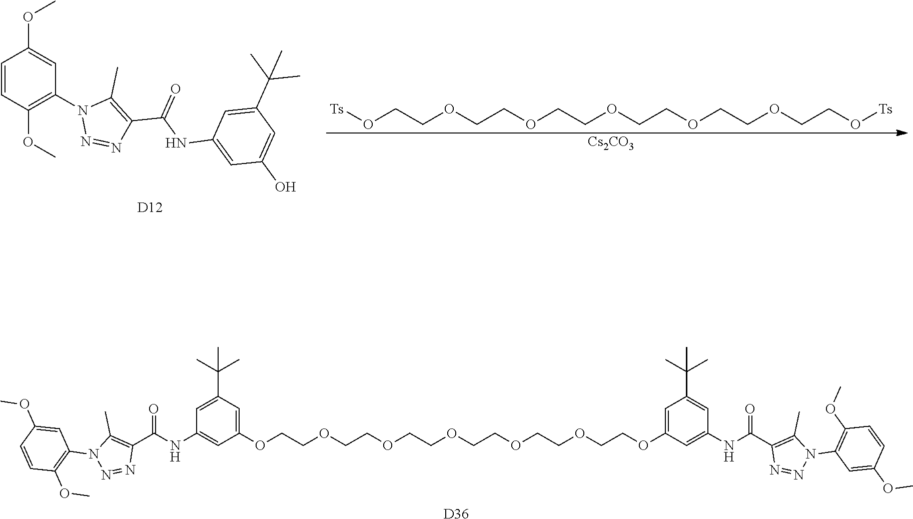

To a solution of N-(3-(tert-butyl)-5-hydroxyphenyl)-1-(2,5-dimethoxyphenvl)-5-methyl-1H-1,2,3-triazole-4-carboxamide (150 mg, 0.365 mmol) in DMF 5 mL was added 3,6,9,12,15-pentaoxaheptadecane-1,17-diyl bis(4-methylbenzenesulfonate) (162 mg, 0.274 mmol) and Cs₂CO₃ (143 mg, 0.439 mmol). Then the reaction mixture was stirred at room temperature for overnight. Then the reaction mixture was diluted with water (50 mL) and extracted with EtOAc (50 mL×2). The combine EtOAc layers were washed with water, dried with anhydrous Na₂SO₄, and concentrated. The residue was purified by silica gel chromatography (0% to 100% acetonitrile in water) to give product N,N-(((3,6,9,12,15-pentaoxaheptadecane-1,17-diyl)bis(oxy))bis(5-(tert-butyl)-3,1-phenylene))bis(1-(2,5-dimethoxyphenyl)-5-methyl-1H-1,2,3-triazole-4-carboxamide) (78.6 mg, 40% yield). ¹H NMR (400 MHz, Chloroform-d) δ 9.07 (s, 2H), 7.40 (t, J=2.1 Hz, 2H), 7.15 (t, J=1.7 Hz, 2H), 7.08 (dd, J=9.1, 3.0 Hz, 2H), 7.02 (d, J=9.1 Hz, 2H), 6.95 (d, J=3.0 Hz, 2H), 6.77 (t, J=1.9 Hz, 2H), 4.17 (t, J=4.9 Hz, 4H), 3.86 (dd, J=5.7, 4.0 Hz, 4H), 3.81 (s, 6H), 3.75 (s, 6H), 3.74-3.71 (m, 4H), 3.70-3.65 (m, 12H), 2.51 (s, 6H), 1.31 (s, 18H). ¹³C NMR (101 MHz, CDCl3) δ 159.32, 159.16, 153.70, 153.44, 148.03, 139.39, 138.52, 137.96, 124.33, 117.50, 113.81, 113.34, 109.48, 109.21, 102.51, 70.81, 70.65, 70.61, 69.79, 67.46, 56.34, 56.01, 34.90, 31.26, 29.28, 9.25. ESI-TOF HRMS: m/z 1067.5450 (C₅₆H₇₄N₈O₁₃+H^{|} requires 1067.5448).

yyyy. Preparation of D38

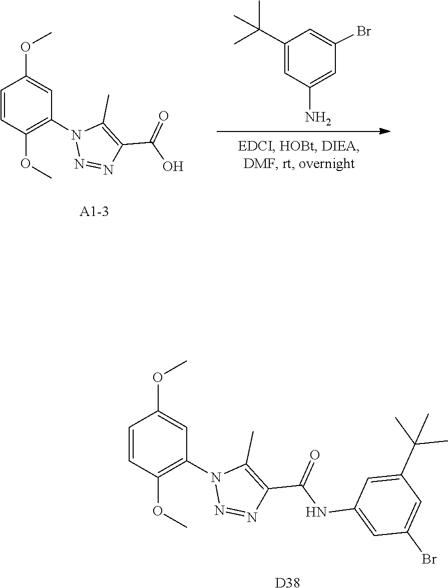 [SMILES: COc1ccc(OC)c(-n2nnc(C(=O)O)c2C)c1 ; CC(C)(C)c1cc(N)cc(Br)c1 ; COc1ccc(OC)c(-n2nnc(C(=O)Nc3cc(Br)cc(C(C)(C)C)c3)c2C)c1 ; "D38"]

The preparation of compound D38 was prepared by reaction of 1-(2,5-dimethoxyphenyl)-5-methyl-1H-1,2,3-triazole-4-carboxylic acid and 3-bromo-5-(tert-butyl)aniline according to the procedure for A1 synthesis. White solid, 17.2 g, 61% yield. ¹H NMR (400 MHz, DMSO-d6) δ 10.50 (s, 1H), 8.09 (t, J=1.8 Hz, 1H), 7.90 (t, J=1.8 Hz, 1H), 7.30-7.26 (m, 2H), 7.24-7.21 (m, 1H), 7.13 (d, J=3.0 Hz, 1H), 3.79 (s, 3H), 3.76 (s, 3H), 3.22 (s, 3H), 2.39 (s, 3H), 1.30 (s, 9H). ¹³C NMR (101 MHz, DMSO) δ 159.69, 153.67, 153.19, 147.79, 139.95, 139.22, 137.34, 123.82, 123.23, 121.24, 119.87, 117.49, 116.58, 114.06, 56.43, 55.93, 34.75, 30.84, 8.76. ESI-TOF HRMS: m/z 473.1186 (C₂₂H₂₅BrN₄O₃+H⁺ requires 473.1183).

zzzz. Preparation of D39

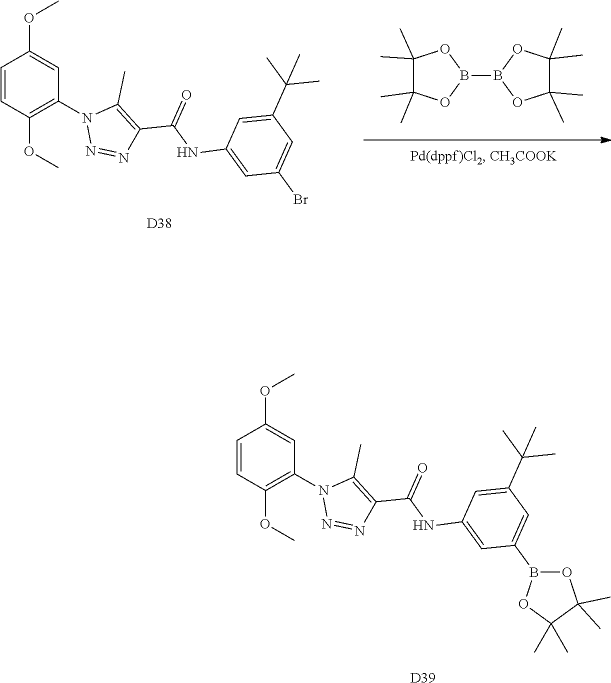

Under N₂, to a degassed solution of N-(3-bromo-5-(tert-butyl)phenyl)-1-(2,5-dimethoxyphenyl)-5-methyl-1H-1,2,3-triazole-4-carboxamide (5 g, 10.56 mmol) in DMF (30 ml) was added CH₃COOK (2.073 g, 21.13 mmol) and 4,4,4′,4′,5,5,5′,5′-octamethyl-2,2′-bi(1,3,2-dioxaborolane) (4.02 g, 15.84 mmol), Pd(dppf)Cl₂ (0.386 g, 0.528 mmol). The reaction mixture was stirred at 100° C. for 8 hours. The reaction mixture was cool down and dilution with EtOAc (100 mL), the suspension is filtered over celite and concentrated. The residue was purified by silica gel chromatography (0% to 100% EtOAc in hexane) to give product as a white solid 5.1 g, 93% yield. ¹H NMR (400 MHz, Chloroform-d) δ 9.08 (s, 1H), 8.03 (t, J=2.1 Hz, 1H), 7.82 (dd, J=2.2, 0.9 Hz, 1H), 7.62 (dd, J=1.9, 0.9 Hz, 1H), 7.08 (dd, J=9.1, 3.0 Hz, 1H), 7.02 (d, J=9.1 Hz, 1H), 6.95 (d, J=2.9 Hz, 1H), 3.82 (s, 3H), 3.75 (s, 3H), 2.52 (s, 3H), 1.38 (s, 9H), 1.35 (s, 12H). ¹³C NMR (101 MHz, CDCl3) δ 159.38, 153.86, 151.63, 148.16, 139.20, 138.05, 137.24, 127.40, 124.66, 123.35, 120.18, 117.50, 113.95, 113.57, 83.76, 56.42, 56.01, 34.83, 31.39, 24.87, 9.14. ESI-TOF HRMS: m/z 521.2922 (C₂₈H₃₇BN₄O₅+H⁺ requires 521.2930).

aaaaa. Preparation of D40

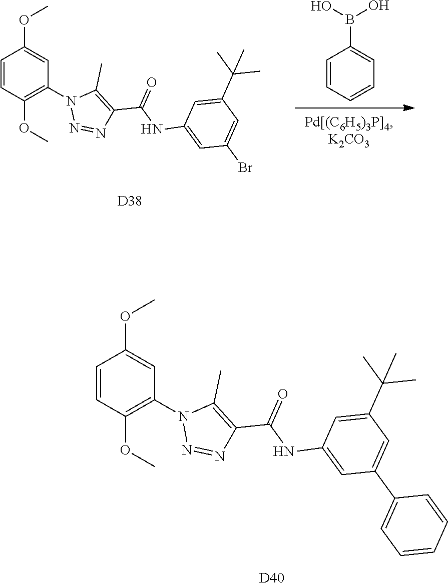 [SMILES: COc1ccc(OC)c(-n2nnc(C(=O)Nc3cc(Br)cc(C(C)(C)C)c3)c2C)c1 ; OB(O)c1ccccc1 ; COc1ccc(OC)c(-n2nnc(C(=O)Nc3cc(-c4ccccc4)cc(C(C)(C)C)c3)c2C)c1 ; "D38" ; "D40"]

Under N₂, K₂CO₃ (58.4 mg, 0.423 mmol), phenylboronic acid (30.9 mg, 0.254 mmol), and palladium-tetrakis(triphenylphosphine) (12.2 mg, 10.56 μmol) were added to a degassed solution of N-(3-bromo-5-(tert-butyl)phenyl)-1-(2,5-dimethoxyphenyl)-5-methyl-1H-1,2,3-triazole-4-carboxamide (100 mg, 0.211 mmol) in DMF (10 ml) and water (5 mL). The reaction mixture was stirred at 100° C. for 8 hours. After cooling down and dilution with EtOAc (100 mL), the suspension is filtered over celite and concentrated. The residue was purified by silica gel chromatography (0% to 100% EtOAc in hexane) to give product as a white solid, 79 mg, 80% yield. ¹H NMR (400 MHz, Chloroform-d) δ 9.15 (s, 1H), 7.89 (t, J=1.8 Hz, 1H), 7.68 (t, J=1.9 Hz, 1H), 7.66 (d, J=1.4 Hz, 1H), 7.65-7.62 (m, 1H), 7.48-7.39 (m, 3H), 7.38-7.32 (m, 1H), 7.10 (dd, J=9.1, 2.9 Hz, 1H), 7.04 (d, J=9.1 Hz, 1H), 6.97 (d, J=3.0 Hz, 1H), 3.83 (s, 3H), 3.77 (s, 3H), 2.54 (s, 3H), 1.42 (s, 9H). ¹³C NMR (101 MHz, CDCl3) δ 159.45, 153.88, 152.72, 148.18, 141.94, 141.50, 139.36, 138.06, 138.03, 128.62, 127.37, 127.25, 124.63, 120.41, 117.52, 116.10, 116.01, 113.99, 113.57, 56.43, 56.02, 34.97, 31.38, 9.17. ESI-TOF HRMS: m/z 471.2395 (C₂₈H₃₀N₄O₃+H⁺ requires 471.2391).

bbbbb. Preparation of D41

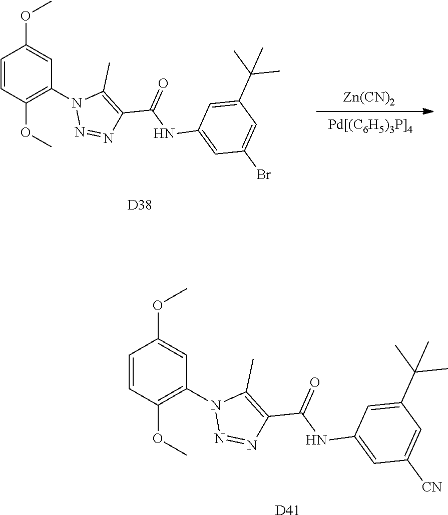

Under N₂, zinc cyanide (0.322 g, 2.75 mmol), and tetrakis(triphenylphosphine)palladium(0) (0.122 g, 0.106 mmol) were added to a degassed solution of N-(3-bromo-5-(tert-butyl)phenyl)-1-(2,5-dimethoxyphenyl)-5-methyl-1H-1,2,3-triazole-4-carboxamide (1 g, 2.113 mmol) in DMF (10 ml). The reaction mixture was stirred at 100° C. for 8 hours. After dilution with EtOAc (100 mL), the suspension is filtered over celite and concentrated. The residue was purified by silica gel chromatography (0% to 100% EtOAc in hexane) to give product as a white solid, 726 mg, 82% yield. ¹H NMR (400 MHz, Chloroform-d) δ 9.16 (s, 1H), 8.07 (dd, J=2.1, 1.4 Hz, 1H), 7.79 (t, J=1.9 Hz, 1H), 7.43 (t, J=1.6 Hz, 1H), 7.10 (dd, J=9.1, 3.0 Hz, 1H), 7.04 (d, J=9.1 Hz, 1H), 6.95 (d, J=3.0 Hz, 1H), 3.82 (s, 3H), 3.76 (s, 3H), 2.52 (s, 3H), 1.36 (s, 9H). ¹³C NMR (101 MHz, CDCl3) δ 159.55, 153.88, 153.72, 148.09, 139.82, 138.47, 137.45, 124.95, 124.40, 121.17, 120.19, 118.92, 117.58, 113.97, 113.55, 112.89, 56.42, 56.02, 35.02, 30.99, 9.12. ESI-TOF HRMS: m/z 420.2025 (C₂₃H₂₅N₅O₃+H⁺ requires 420.2030).

ccccc. Preparation of D42

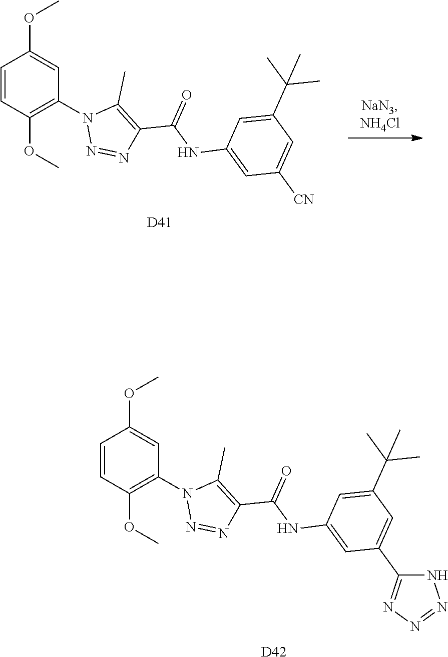 [SMILES: COc1ccc(OC)c(-n2nnc(C(=O)Nc3cc(C#N)cc(C(C)(C)C)c3)c2C)c1 ; "D41" ; COc1ccc(OC)c(-n2nnc(C(=O)Nc3cc(-c4nnn[nH]4)cc(C(C)(C)C)c3)c2C)c1 ; "D42"]

A mixture of N-(3-(tert-butyl)-5-cyanophenyl)-1-(2,5-dimethoxyphenyl)-5-methyl-1H-1,2,3-triazole-4-carboxamide (300 mg, 0.715 mmol), sodium azide (186 mg, 2.86 mmol) and ammonium chloride (153 mg, 2.86 mmol) was stirred in 10 mL DMSO at 100° C. for 18 hours. The mixture was cool down and extracted with EtOAc (100 mL). The organic layer was washed with brine, dried over anhydrous Na₂SO₄ concentrated. The crude product was purified by column chromatography (0% to 100% EtOAc in hexane) to provide product as a white solid, 135.2 mg, 41% yield. ¹H NMR (400 MHz, Chloroform-d) δ 9.31 (s, 1H), 8.36 (s, 1H), 7.99 (s, 1H), 7.70 (s, 1H), 7.14-6.98 (m, 2H), 7.00-6.90 (m, 1H), 3.80 (s, 3H), 3.74 (s, 3H), 2.48 (s, 3H), 1.34 (s, 9H). ¹³C NMR (101 MHz, CDCl3) δ 160.04, 154.04, 153.86, 148.05, 139.91, 137.95, 137.48, 124.24, 121.17, 120.21, 117.66, 116.27, 113.95, 113.53, 56.40, 56.03, 35.08, 31.09, 9.19. ESI-TOF HRMS: m/z 463.2203 (C₂₃H₂₆N₈O₃+H⁺ requires 463.2201).

ddddd. Preparation of E1

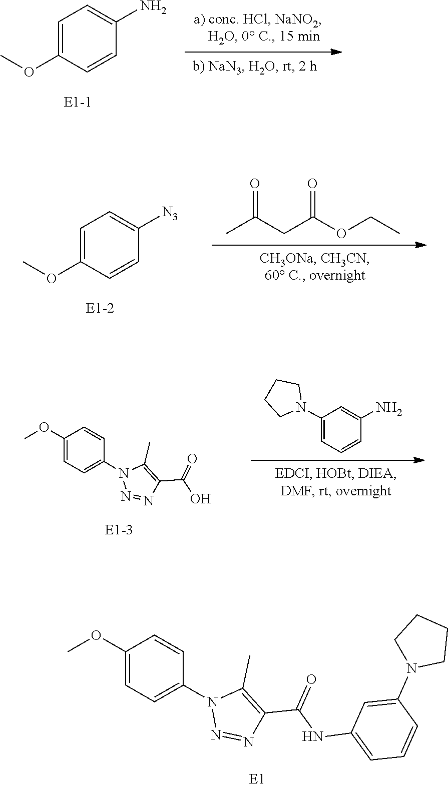

To a solution of 4-methoxyaniline (12.32 g, 100 mmol) in water (100 ml)/hexane (100 mL) was added hydrochloric Acid, 37% (24.00 ml). The reaction mixture was cooled to 0° C. Sodium nitrite (6.90 g, 100 mmol) dissolved into water (20.00 ml) was added to the reaction mixture. After being stirred for 10 mins, sodium azide (7.15 g, 110 mmol) in water (20.00 ml) was added dropwise to the reaction mixture. The mixture was stirred at room temperature for 1.5 hours. The mixture was extracted with hexane (200 mL). The organic phase was washed with water (200 mL) and once with brine (200 mL). The organic layer was dried over anhydrous MgSO₄, filtered and concentrated to get a brown oil. The resulting aryl azides were used without further purification.

To the residue was added ethyl acetoacetate (15.30 ml, 120 mmol), sodium methanolate (21.61 g, 400 mmol) and MeOH 100 mL. The mixture was stirred at 60° C. for overnight. Then the reaction mixture was evaporated under reduced pressure. The residue was added sodium hydroxide (16.00 g, 400 mmol) and water (400 mL) and stirred at room temperature for 3 hours. Then the pH was adjusted to around 4 with hydrochloric acid (37%). The mixture was extracted with ethyl acetate (2×500 mL) and dried over MgSO₄, filtered, and concentrated under reduced pressure to provide 1-(4-methoxyphenyl)-5-methyl-1H-1,2,3-triazole-4-carboxylic acid (4.32 g, 19% yield) as a brown solid. ¹H NMR (400 MHz, DMSO-d6) δ 7.59-7.50 (m, 2H), 7.19-7.10 (m, 2H), 3.85 (s, 3H), 2.46 (s, 3H). ¹³C NMR (101 MHz, DMSO) δ 162.63, 160.13, 138.97, 136.32, 128.07, 126.95, 114.70, 55.58, 9.62.

To a solution of 1-(4-methoxyphenyl)-5-methyl-1H-1,2,3-triazole-4-carboxylic acid (0.233 g, 1 mmol) in DMF 5 mL was added 3-(pyrrolidin-1-yl)aniline (0.162 g, 1.000 mmol), HOBt, 80% (0.203 g, 1.200 mmol) and EDCI (0.288 g, 1.500 mmol), followed by N-ethyl-N-isopropylpropan-2-amine (0.331 ml, 2.000 mmol). The suspension was stirred at room temperature for overnight. The solvent was diluted with water and extracted with ethyl acetate (2×50 mL). The organic solvent washed by NaHCO₃ saturated solution, water and brine, dried by MgSO₄, filtered and concentrated. The residue purified to providing 1-(4-methoxyphenyl)-5-methyl-N-(3-(pyrrolidin-1-yl)phenyl)-1H-1,2,3-triazole-4-carboxamide (256.3 mg, 68% yield) as a white solid. ¹H NMR (400 MHz, DMSO-d6) δ 10.12 (s, 1H), 7.60-7.52 (m, 2H), 7.21-7.13 (m, 4H), 7.09 (dd, J=8.7, 7.4 Hz, 1H), 6.29 (dt, J=8.0, 1.5 Hz, 1H), 3.86 (s, 3H), 3.26-3.19 (m, 4H), 2.53 (s, 3H), 2.00-1.89 (m, 4H). ¹³C NMR (101 MHz, DMSO) δ 160.16, 159.29, 148.01, 139.33, 138.19, 137.45, 128.90, 128.07, 126.94, 114.73, 107.48, 107.45, 103.68, 55.60, 47.31, 24.91, 9.36. ESI-TOF HRMS: m/z 378.1918 (C21H23N₅O2+H⁺ requires 378.1925).

eeeee. Preparation of E2

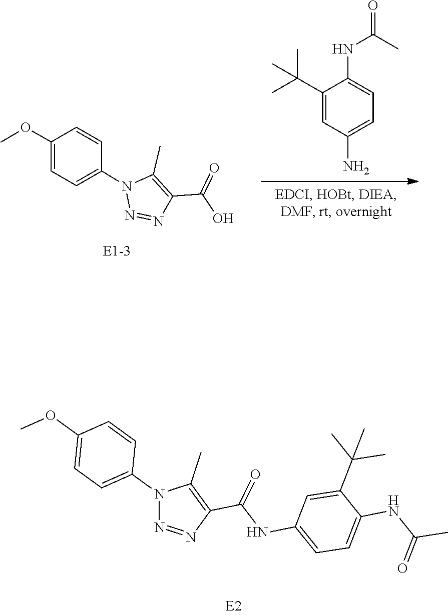 [SMILES: COc1ccc(-n2nnc(C(=O)O)c2C)cc1 ; CC(=O)Nc1ccc(N)cc1C(C)(C)C ; COc1ccc(-n2nnc(C(=O)Nc3ccc(NC(C)=O)c(C(C)(C)C)c3)c2C)cc1]

The preparation of compound E-2 was prepared by reaction of 1-(4-methoxyphenyl)-5-methyl-1H-1,2,3-triazole-4-carboxylic acid and N-(4-amino-2-(tert-butyl)phenyl)acetamide according to the procedure for A1 synthesis. White solid, 371.0 mg, 88% yield. ¹H NMR (400 MHz, DMSO-d6) δ 10.44 (s, 1H), 9.25 (s, 1H), 7.68-7.61 (m, 2H), 7.61-7.53 (m, 2H), 7.33 (d, J=9.1 Hz, 1H), 7.22-7.13 (m, 2H), 3.86 (s, 3H), 2.53 (s, 3H), 2.04 (s, 3H), 1.31 (s, 9H). ¹³C NMR (101 MHz, DMSO) δ 168.98, 160.18, 159.40, 141.78, 137.99, 137.64, 136.81, 136.00, 128.05, 126.97, 126.50, 123.40, 118.65, 114.72, 55.60, 34.38, 30.79, 23.11, 9.35. ESI-TOF HRMS: m/z 422.2195 (C₂₃H₂₇N₅O₃+H⁺ requires 422.2187).

fffff. Preparation of E3

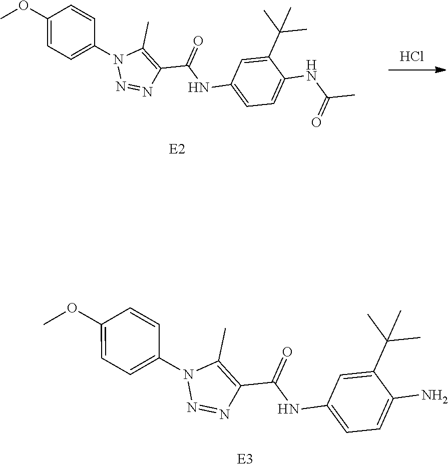

To a solution of N-(4-acetamido-3-(tert-butyl)phenyl)-1-(4-methoxyphenyl)-5-methyl-1H-1,2,3-triazole-4-carboxamide (0.126 g, 0.3 mmol) in water (10 mL) and EtOH (5 mL) was added 12 M hydrochloric acid (2 mL, 6 mmol). The suspension was stirred 80° C. for overnight. After the reaction mixture was cool down the pH was adjusted to more than 7 by NaHCO₃ saturated solution. The reaction mixture was diluted with water and extracted with EtOAc (100 mL). The organic solvent washed by NaHCO₃ saturated solution, water and brine, dried by MgSO₄, filtered and concentrated. The residue was purified to providing N-(4-amino-3-(tert-butyl)phenyl)-1-(4-methoxyphenyl)-5-methyl-1H-1,2,3-triazole-4-carboxamide (81.5 mg, 67% yield) as a white solid. ¹H NMR (400 MHz, DMSO-d6) δ 9.99 (s, 1H), 7.58-7.53 (m, 2H), 7.22 (d, J=2.3 Hz, 1H), 7.20-7.14 (m, 2H), 7.00 (d, J=8.6 Hz, 1H), 6.88 (dd, J=8.5, 2.3 Hz, 1H), 4.77 (s, 2H), 3.86 (s, 3H), 2.52 (s, 3H), 1.33 (s, 9H). 13C NMR (101 MHz, DMSO) δ 160.15, 159.05, 146.01, 138.21, 137.32, 136.78, 128.10, 128.03, 126.95, 125.90, 114.73, 108.97, 108.87, 55.60, 33.58, 29.37, 9.31. ESI-TOF HRMS: m/z 422.2195 (C₂₃H₂₇N₅O₃+H⁺ requires 422.2187).

ggggg. Preparation of E4

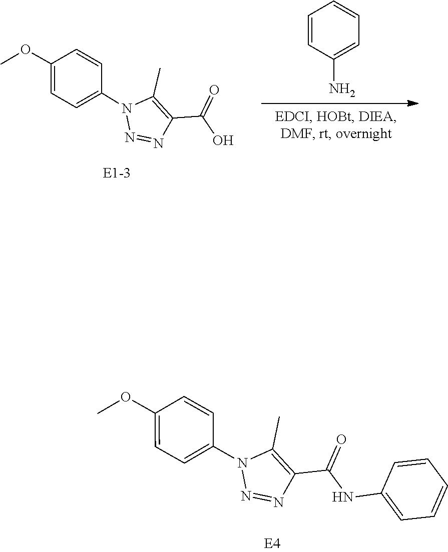 [SMILES: COc1ccc(-n2nnc(C(=O)O)c2C)cc1 ; Nc1ccccc1 ; COc1ccc(-n2nnc(C(=O)Nc3ccccc3)c2C)cc1]

The preparation of compound E4 was prepared by reaction of 1-(4-methoxyphenyl)-5-methyl-1H-1,2,3-triazole-4-carboxylic acid and aniline according to the procedure for A1 synthesis. White solid, 138.5 mg, 45% yield. ¹H NMR (400 MHz, DMSO-d₆) δ 10.46 (s, 1H), 7.89-7.83 (m, 2H), 7.62-7.53 (m, 2H), 7.37-7.31 (m, 2H), 7.21-7.15 (m, 2H), 7.13-7.07 (m, 1H), 3.86 (s, 3H), 2.54 (s, 3H). ESI-TOF HRMS: m/z 309.1346 (C₁₇H₁₆N₄O₂+H⁺ requires 309.1346).

hhhhh. Preparation of E5

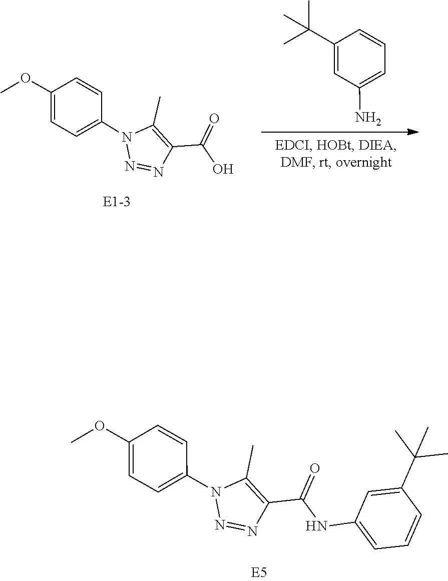 [SMILES: COc1ccc(-n2nnc(C(=O)O)c2C)cc1 ; CC(C)(C)c1cccc(N)c1 ; COc1ccc(-n2nnc(C(=O)Nc3cccc(C(C)(C)C)c3)c2C)cc1]

The preparation of compound E5 was prepared by reaction of 1-(4-methoxyphenyl)-5-methyl-1H-1,2,3-triazole-4-carboxylic acid and 3-(tert-butyl)aniline according to the procedure for A1 synthesis. White solid, 273.5 mg, 75% yield. ¹H NMR (400 MHz, DMSO-d6) δ 10.37 (s, 1H), 7.92 (t, J=2.0 Hz, 1H), 7.74 (ddd, J=8.0, 2.1, 1.0 Hz, 1H), 7.60-7.54 (m, 2H), 7.26 (t, J=7.9 Hz, 1H), 7.20-7.15 (m, 2H), 7.13 (ddd, J=7.9, 2.0, 1.1 Hz, 1H), 3.86 (s, 3H), 2.54 (s, 3H), 1.29 (s, 9H). ¹³C NMR (101 MHz, DMSO) δ 160.17, 159.45, 151.11, 138.37, 138.09, 137.56, 128.12, 128.06, 126.95, 120.51, 117.52, 117.44, 114.73, 55.60, 34.46, 31.11, 9.37. ESI-TOF HRMS: m/z 365.1966 (C₂₁H₂₄N₄O₂+H⁺ requires 365.1972).

iiiii. Preparation of E6

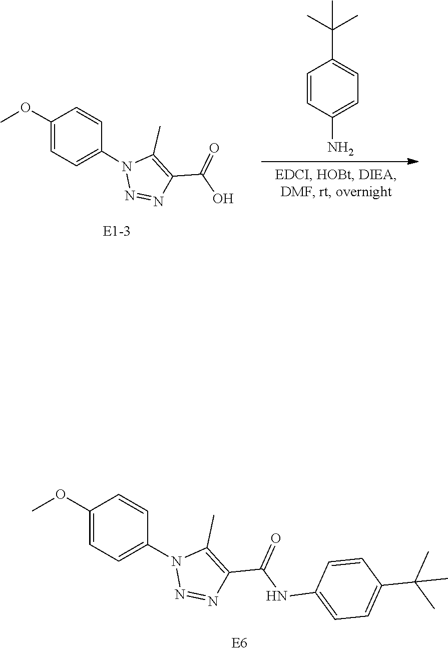 [SMILES: COc1ccc(-n2nnc(C(=O)O)c2C)cc1 ; CC(C)(C)c1ccc(N)cc1 ; COc1ccc(-n2nnc(C(=O)Nc3ccc(C(C)(C)C)cc3)c2C)cc1]

The preparation of compound E6 was prepared by reaction of 1-(4-methoxyphenyl)-5-methyl-1H-1,2,3-triazole-4-carboxylic acid and 4-(tert-butyl)aniline according to the procedure for A1 synthesis. White solid, 215.6 mg, 59% yield. ¹H NMR (400 MHz, DMSO-d₆) δ 10.38 (s, 1H), 7.80-7.72 (m, 2H), 7.61-7.53 (m, 2H), 7.39-7.31 (m, 2H), 7.22-7.13 (m, 2H), 3.86 (s, 3H), 2.53 (s, 3H), 1.28 (s, 9H). ¹¹C NMR (101 MHz, DMSO) δ 160.17, 159.35, 145.97, 138.05, 137.54, 135.99, 128.07, 126.96, 125.14, 120.18, 114.73, 55.60, 34.02, 31.17, 9.36. ESI-TOF HRMS: m/z 365.1965 (C₂₁H₂₄N₄O₂+H⁺ requires 365.1972).

jjjjj. Preparation of E7

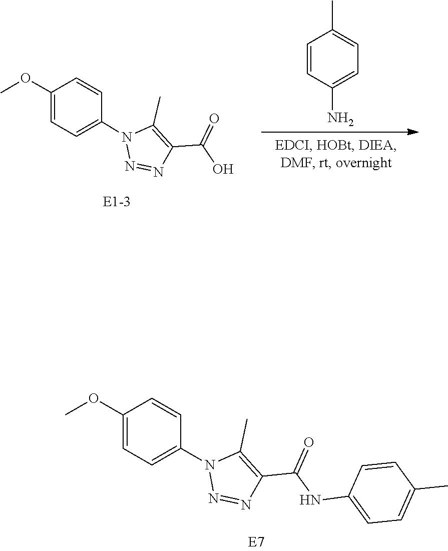

The preparation of compound E7 was prepared by reaction of 1-(4-methoxyphenyl)-5-methyl-1H-1,2,3-triazole-4-carboxylic acid and according to the procedure for A1 synthesis. White solid, 278.5 mg, 86% yield. ¹H NMR (500 MHz, CDCl₃) δ 9.08 (s, 1H), 7.76-7.68 (m, 2H), 7.38 (ddd, J=8.4, 4.6, 1.9 Hz, 4H), 7.15 (tt, J=7.4, 1.2 Hz, 1H), 7.11-7.03 (m, 2H), 3.90 (s, 3H), 2.64 (s, 3H). ¹³C NMR (126 MHz, CDCl₃) δ 160.76, 159.29, 138.39, 137.72, 137.57, 129.11, 128.30, 126.73, 124.33, 119.80, 114.83, 55.71, 9.78. ESI-TOF HRMS: m/z 323.1501 (C₁₈H₁₈N₄O₂+H⁺ requires 323.1503).

kkkkk. Preparation of E8

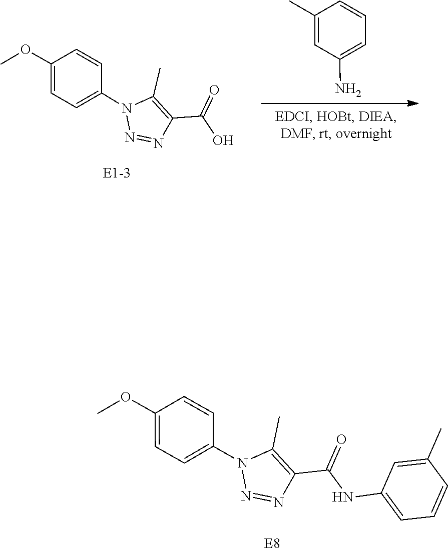

The preparation of compound E8 was prepared by reaction of 1-(4-methoxyphenyl)-5-methyl-1H-1,2,3-triazole-4-carboxylic acid and m-toluidine according to the procedure for A1 synthesis. White solid, 261.2 mg, 810% yield. ¹H NMR (400 MHz, DMSO-d6) δ 10.35 (s, 1H), 7.73 (d, J=2.0 Hz, 1H), 7.66-7.60 (m, 1H), 7.60-7.53 (m, 2H), 7.22 (t, J=7.8 Hz, 1H), 7.19-7.15 (m, 2H), 6.92 (ddd, J=7.4, 1.8, 1.0 Hz, 1H), 3.86 (s, 3H), 2.53 (s, 3H), 2.31 (s, 3H). ¹³C NMR (101 MHz, DMSO) δ 160.18, 159.45, 138.52, 138.05, 137.69, 137.61, 128.37, 128.05, 126.95, 124.33, 120.85, 117.54, 114.73, 55.60, 21.21, 9.35. ESI-TOF HRMS: m/z 323.1504 (C₁₈H₁₈N₄O₂+H⁺ requires 323.1503).

lllll. Preparation of E9

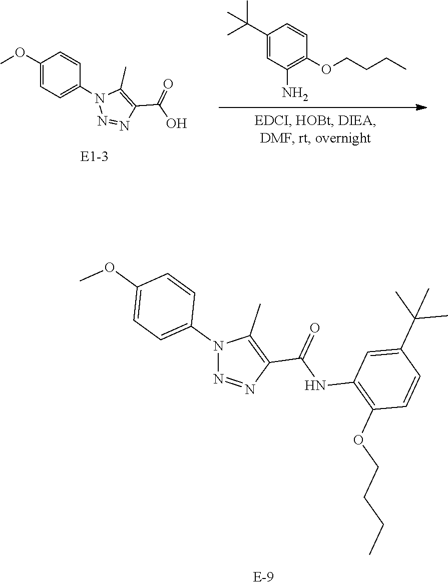 [SMILES: COc1ccc(-n2nnc(C(=O)O)c2C)cc1 ; CCCCOc1ccc(C(C)(C)C)cc1N ; CCCCOc1ccc(C(C)(C)C)cc1NC(=O)c1nnn(-c2ccc(OC)cc2)c1C]

The preparation of compound E9 was prepared by reaction of 1-(4-methoxyphenyl)-5-methyl-1H-1,2,3-triazole-4-carboxylic acid and 2-butoxy-5-(tert-butyl)aniline according to the procedure for A1 synthesis. White solid, 315.7 mg, 72% yield. ¹H NMR (400 MHz, Chloroform-d) δ 9.81 (s, 1H), 8.64 (d, J=2.4 Hz, 1H), 7.42-7.36 (m, 2H), 7.07 (dq, J=8.2, 3.4, 2.8 Hz, 3H), 6.86 (d, J=8.5 Hz, 1H), 4.08 (t, J=6.5 Hz, 2H), 3.89 (s, 3H), 2.66 (s, 3H), 1.88 (dq, J=8.2, 6.5 Hz, 2H), 1.64-1.53 (m, 2H), 1.36 (s, 9H), 1.01 (t, J=7.4 Hz, 3H). ¹³C NMR (101 MHz, CDCl3) δ 160.64, 159.29, 145.72, 143.80, 139.04, 137.04, 128.50, 127.47, 126.70, 120.18, 117.03, 114.76, 110.61, 68.52, 55.67, 34.44, 31.59, 31.30, 19.24, 13.87, 9.81. ESI-TOF HRMS: m/z 437.2543 (C₂₅H₃₂N₄O₃+H⁺ requires 437.2547).

mmmmm. Preparation of E10

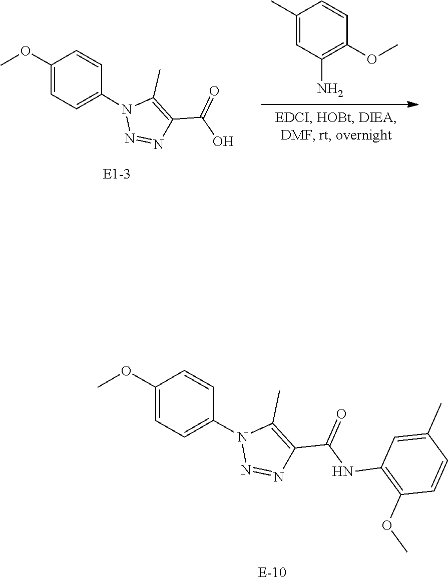

The preparation of compound E10 was prepared by reaction of 1-(4-methoxyphenyl)-5-methyl-1H-1,2,3-triazole-4-carboxylic acid and 2-methoxy-5-methylaniline according to the procedure for A1 synthesis. White solid, 137.8 mg, 91% yield. ¹H NMR (500 MHz, DMSO-d6) δ 9.55 (s, 1H), 8.17 (d, J=2.1 Hz, 1H), 7.63-7.55 (m, 2H), 7.22-7.14 (m, 2H), 7.01 (d, J=8.3 Hz, 1H), 6.92 (ddd, J=8.3, 2.2, 0.9 Hz, 1H), 3.90 (s, 3H), 3.86 (s, 3H), 2.55 (s, 3H), 2.28 (s, 3H). ¹³C NMR (126 MHz, DMSO) δ 160.75, 158.94, 146.75, 138.14, 138.12, 129.89, 128.44, 127.46, 127.09, 124.75, 120.43, 115.26, 111.30, 56.56, 56.12, 21.09, 9.76. ESI-TOF HRMS: m/z 353.1614 (C₁₉H₂₀N₄O₃+H⁺ requires 353.1608).

nnnnn. Preparation of F1

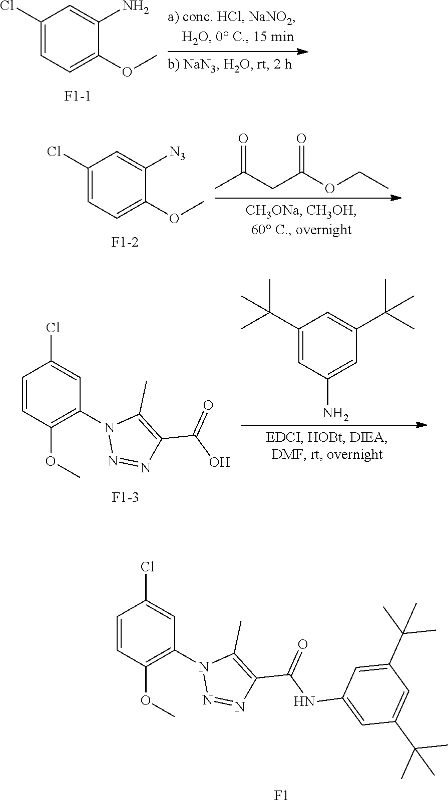

The preparation of compound F1 was prepared by 5-chloro-2-methoxyaniline as the starting compound to get 1-(5-chloro-2-methoxyphenyl)-5-methyl-1H-1,2,3-triazole-4-carboxylic acid and then reaction with 3,5-di-tert-butylaniline according to the procedure for A1 synthesis. White solid, 155.9 mg, 69% yield. ¹H NMR (400 MHz, Chloroform-d) δ 9.07 (s, 1H), 7.59 (d, J=1.7 Hz, 2H), 7.51 (dt, J=8.9, 1.7 Hz, 1H), 7.43 (d, J=2.6 Hz, 1H), 7.23 (t, J=1.7 Hz, 1H), 7.05 (d, J=8.9 Hz, 1H), 3.82 (s, 3H), 2.53 (s, 3H), 1.36 (s, 18H). ¹³C NMR (101 MHz, CDCl3) δ 159.14, 152.75, 151.74, 139.28, 138.21, 137.18, 131.92, 128.58, 125.94, 124.88, 118.50, 114.39, 113.29, 56.23, 35.00, 31.46, 9.25. ESI-TOF HRMS: m/z 455.2207 (C₂₅H₃₁ClN₄O₂+H⁺ requires 455.2209).

ooooo. Preparation of F2

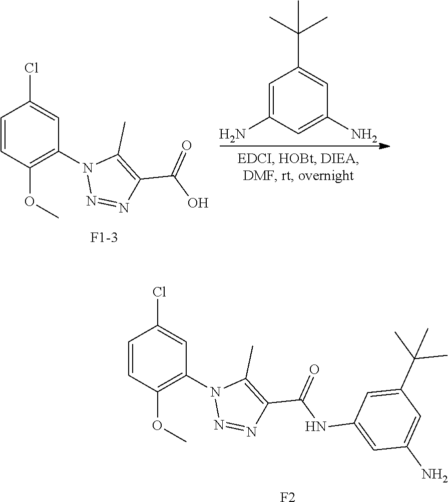 [SMILES: COc1ccc(Cl)cc1-n1nnc(C(=O)O)c1C ; CC(C)(C)c1cc(N)cc(N)c1 ; COc1ccc(Cl)cc1-n1nnc(C(=O)Nc2cc(N)cc(C(C)(C)C)c2)c1C ; "F2"]

The preparation of compound F2 was prepared by reaction of 1-(5-chloro-2-methoxyphenyl)-5-methyl-1H-1,2,3-triazole-4-carboxylic acid (0.535 g, 2 mmol) and 5-(tert-butyl)benzene-1,3-diamine according to the procedure for A1 synthesis. Light brown solid, 572.3 mg, 69% yield. ¹H NMR (500 MHz, CDCl₃) δ 9.01 (s, 1H), 7.52 (dd, J=9.0, 2.6 Hz, 1H), 7.41 (d, J=2.6 Hz, 1H), 7.37 (t, J=2.0 Hz, 1H), 7.07-7.02 (m, 2H), 6.69 (t, J=1.8 Hz, 1H), 3.81 (s, 3H), 2.50 (s, 3H), 1.30 (s, 9H). ¹³C NMR (126 MHz, CDCl₃) δ 159.19, 153.64, 152.73, 143.68, 139.45, 138.44, 138.03, 131.94, 128.57, 125.93, 124.82, 113.27, 110.15, 109.40, 105.58, 56.24, 34.80, 31.24, 9.23. ESI-TOF HRMS: m/z 414.1685 (C₂₁H₂₄ClN₅O₂+H⁺ requires 414.1692).

ppppp. Preparation of F3

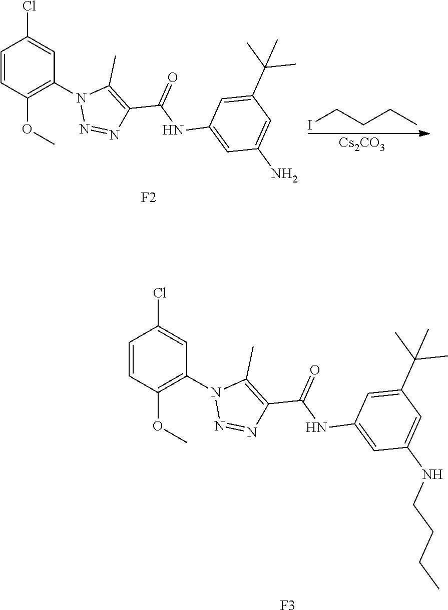

The preparation of compound F3 was prepared by reaction of N-(3-amino-5-(tert-butyl)phenyl)-1-(5-chloro-2-methoxyphenyl)-5-methyl-1H-1,2,3-triazole-4-carboxamide and 1-iodobutane to the procedure for D2 synthesis. White solid, 129.9 mg, 69% yield. ¹H NMR (400 MHz, Chloroform-d) δ 8.98 (s, 1H), 7.52 (dd, J=8.9, 2.6 Hz, 1H), 7.42 (d, J=2.6 Hz, 1H), 7.09 (t, J=2.0 Hz, 1H), 7.04 (d, J=8.9 Hz, 1H), 6.92 (t, J=1.8 Hz, 1H), 6.45 (t, J=1.9 Hz, 1H), 3.82 (s, 3H), 3.16 (t, J=7.1 Hz, 2H), 2.52 (s, 3H), 1.69-1.57 (m, 2H), 1.50-1.39 (m, 2H), 1.31 (s, 9H), 0.97 (t, J=7.3 Hz, 3H). ¹³C NMR (101 MHz, CDCl3) δ 159.09, 153.07, 152.76, 148.87, 139.26, 138.49, 138.24, 131.90, 128.58, 125.93, 124.89, 113.27, 106.68, 106.35, 101.38, 56.23, 43.89, 34.77, 31.70, 31.30, 20.33, 13.95, 9.23. ESI-TOF HRMS: m/z 470.2308 (C₂₅H₃₂ClN₅O₂+H⁺ requires 470.2318).

qqqqq. Preparation of F4

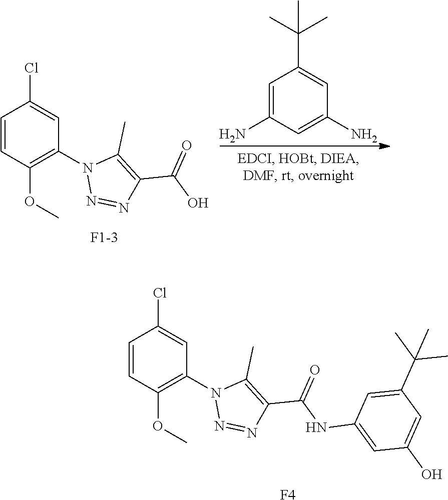 [SMILES: COc1ccc(Cl)cc1-n1nnc(C(=O)O)c1C ; CC(C)(C)c1cc(N)cc(N)c1 ; COc1ccc(Cl)cc1-n1nnc(C(=O)Nc2cc(O)cc(C(C)(C)C)c2)c1C ; "F4"]

The preparation of compound F4 was prepared by reaction of 1-(5-chloro-2-methoxyphenyl)-5-methyl-1H-1,2,3-triazole-4-carboxylic acid (0.535 g, 2 mmol) and 3-amino-5-(tert-butyl)phenol according to the procedure for A1 synthesis. White solid, 892.1 mg, 72% yield. ¹H NMR (400 MHz, Chloroform-d) δ 9.12 (s, 1H), 7.56 (q, J=2.0 Hz, 1H), 7.53 (dd, J=8.9, 2.6 Hz, 1H), 7.42 (d, J=2.6 Hz, 1H), 7.05 (d, J=8.9 Hz, 1H), 6.99 (t, J=1.7 Hz, 1H), 6.71 (dd, J=2.3, 1.6 Hz, 1H), 3.82 (s, 3H), 2.52 (s, 3H), 1.30 (s, 9H). ¹³C NMR (101 MHz, CDCl3) δ 159.45, 156.44, 153.87, 152.76, 139.67, 138.11, 137.93, 132.02, 128.57, 125.96, 124.73, 113.30, 109.16, 109.13, 104.66, 56.26, 34.81, 31.22, 9.26. ESI-TOF HRMS: m/z 415.1527 (C₂₁H₂₃ClN₄O₃+H^{|} requires 415.1532).

rrrrr. Preparation of F5

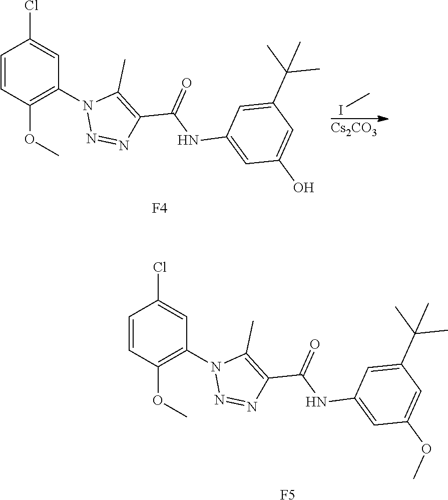

To a solution of N-(3-(tert-butyl)-5-methoxyphenyl)-1-(5-chloro-2-methoxyphenyl)-5-methyl-1H-1,2,3-triazole-4-carboxamide (0.172 g, 0.4 mmol) in DMF 5 mL was added iodomethane (0.068 g, 0.480 mmol) and Cs₂CO₃ (0.156 g, 0.480 mmol). The suspension was stirred at room temperature for overnight. Then the reaction mixture was diluted with water (50 mL) and extracted with EtOAc (50 mL×2). The EtOAc layer was washed with water, dried with anhydrous Na₂SO₄, and concentrated. The residue was purified by silica gel chromatography (0% to 100% EA in hexane) to give product as white solid (144.0 mg, 84% yield). ¹H NMR (400 MHz, Chloroform-L) δ 9.07 (s, 1H), 7.52 (ddd, J=8.9, 2.6, 0.9 Hz, 1H), 7.44-7.38 (m, 2H), 7.16 (t, J=1.8 Hz, 1H), 7.05 (d, J=8.9 Hz, 1H), 6.75 (dd, J=2.4, 1.6 Hz, 1H), 3.85 (s, 3H), 3.82 (s, 3H), 2.52 (s, 3H), 1.33 (s, 9H). ¹³C NMR (101 MHz, CDCl3) δ 159.98, 159.18, 153.52, 152.74, 139.40, 138.53, 138.06, 131.96, 128.57, 125.96, 124.82, 113.29, 109.39, 108.65, 101.82, 56.24, 55.37, 34.90, 31.26, 9.23.

ESI-TOF HRMS: m/z 429.1692 (C₂₂H₂₅ClN₄O₃+H⁺ requires 429.1688).

sssss. Preparation of F6

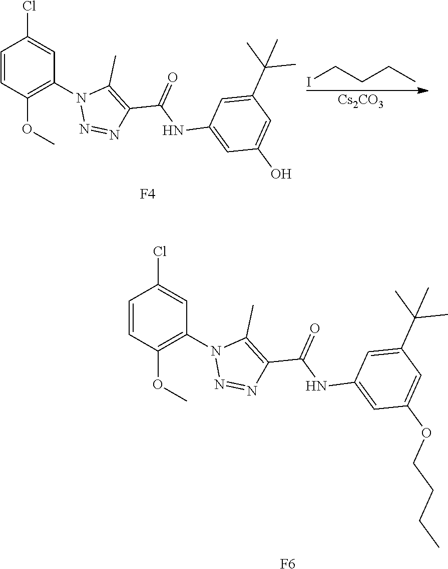

The preparation of compound F6 was prepared by reaction of N-(3-(tert-butyl)-5-methoxyphenyl)-1-(5-chloro-2-methoxyphenyl)-5-methyl-1H-1,2,3-triazole-4-carboxamide and 1-iodobutane according to the procedure for F5 synthesis. White solid, 149.6 mg, 80% yield. ¹H NMR (400 MHz, Chloroform-d) δ 9.06 (s, 1H), 7.52 (ddd, J=8.9, 2.6, 0.8 Hz, 1H), 7.45-7.36 (m, 2H), 7.13 (t, J=1.8 Hz, 1H), 7.05 (d, J=8.9 Hz, 1H), 6.75 (dd, J=2.3, 1.6 Hz, 1H), 4.01 (t, J=6.5 Hz, 2H), 3.82 (s, 3H), 2.52 (s, 3H), 1.78 (dq, J=8.0, 6.6 Hz, 2H), 1.59-1.45 (m, 2H), 1.33 (s, 9H), 0.99 (t, J=7.4 Hz, 3H). ¹³C NMR (101 MHz, CDCl3) δ 159.58, 159.15, 153.43, 152.74, 139.37, 138.45, 138.09, 131.95, 128.57, 125.96, 124.83, 113.29, 109.19, 109.13, 102.41, 67.73, 56.24, 34.89, 31.46, 31.28, 19.31, 13.92, 9.23.

ESI-TOF HRMS: m/z 471.2154 (C₂₅H₃₁ClN₄O₃+H⁺ requires 471.2158).

ttttt. Preparation of F7

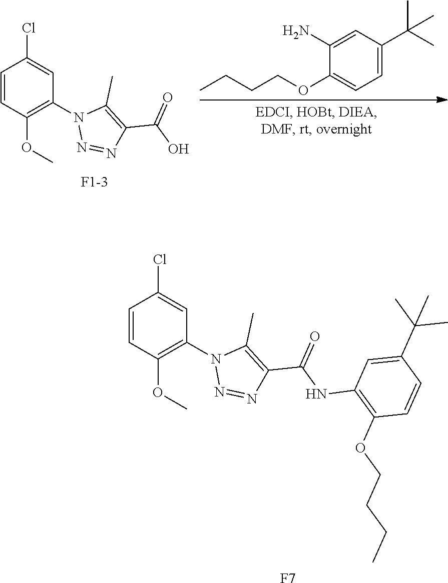

The preparation of compound F4 was prepared by reaction of 1-(5-chloro-2-methoxyphenyl)-5-methyl-1H-1,2,3-triazole-4-carboxylic acid (0.535 g, 2 mmol) and 2-butoxy-5-(tert-butyl)aniline according to the procedure for A1 synthesis. White solid, 378.9 mg, 80% yield. ¹H NMR (400 MHz, Chloroform-d) δ 9.81 (s, 1H), 8.65 (d, J=2.4 Hz, 1H), 7.49 (dd, J=8.9, 2.6 Hz, 1H), 7.42 (d, J=2.6 Hz, 1H), 7.07 (dd, J=8.5, 2.4 Hz, 1H), 7.03 (d, J=8.9 Hz, 1H), 6.86 (d, J=8.6 Hz, 1H), 4.08 (t, J=6.4 Hz, 2H), 3.80 (s, 3H), 2.54 (s, 3H), 1.88 (dq, J=8.4, 6.5 Hz, 2H), 1.66-1.53 (m, 2H), 1.36 (s, 9H), 1.00 (t, J=7.4 Hz, 3H). ¹³C NMR (101 MHz, CDCl3) δ 159.22, 152.73, 145.71, 143.78, 139.06, 138.59, 131.82, 128.55, 127.46, 125.88, 124.98, 120.20, 117.02, 113.29, 110.61, 68.52, 56.22, 34.45, 31.59, 31.32, 19.26, 13.88, 9.25. ESI-TOF HRMS: m/z 471.2154 (C₂₅H₃₁ClN₄O₃+H⁺ requires 471.2158).

uuuuu. Preparation of F8

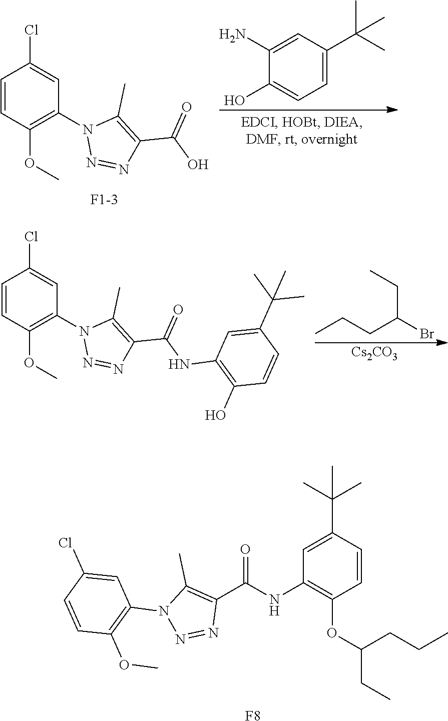

To a solution of 1-(5-chloro-2-methoxyphenyl)-5-methyl-1H-1,2,3-triazole-4-carboxylic acid (324 mg, 1.210 mmol) in DMF (5 mL) was added 2-amino-4-(tert-butyl)phenol (200 mg, 1.210 mmol), HOBt, 80% (307 mg, 1.816 mmol) and EDCI (348 mg, 1.816 mmol), followed by N-ethyl-N-isopropylpropan-2-amine (626 mg, 4.84 mmol). The suspension stirred at room temperature for overnight. The reaction mixture was diluted by (50 mL) and extracted with EtOAc (50 mL×2). The EtOAc layer was washed with water, dried with anhydrous Na₂SO₄ and concentrated. The residue was purified by silica gel chromatography (0% to 100% acetonitrile in water) to give product as a white solid (351.9 mg, 70% yield). ¹H NMR (500 MHz, DMSO-d6) δ 10.04 (s, 1H), 9.62 (s, 1H), 8.34 (d, J=2.4 Hz, 1H), 7.73 (dq, J=5.0, 2.7 Hz, 2H), 7.43-7.37 (m, 1H), 7.00 (dd, J=8.4, 2.4 Hz, 1H), 6.87 (d, J=8.4 Hz, 1H), 3.84 (s, 3H), 2.43 (s, 3H), 1.28 (s, 9H). ¹³C NMR (126 MHz, DMSO) δ 157.80, 152.22, 143.64, 140.95, 138.48, 136.82, 131.55, 127.71, 125.04, 123.67, 123.64, 120.16, 116.30, 114.02, 113.74, 55.92, 33.36, 30.83, 8.13.

To a solution of N-(5-(tert-butyl)-2-hydroxyphenyl)-1-(5-chloro-2-methoxyphenyl)-5-methyl-1H-1,2,3-triazole-4-carboxamide (100 mg, 0.241 mmol) in acetone (5 mL) was added Cs₂CO₃ (157 mg, 0.482 mmol) and 3-bromohexane (47.7 mg, 0.289 mmol). The suspension was stirred at 60° C. for overnight. The reaction mixture was diluted by water (25 mL) and extracted with EtOAc (25 mL×2). The EtOAc layer was washed with water, dried with anhydrous Na₂SO₄ and concentrated. The residue was purified by silica gel chromatography (0% to 100% EA in hexane) to give product as a white solid (89.8 mg, 75% yield). ¹H NMR (500 MHz, CDCl₃) δ 9.87 (s, 1H), 8.65 (d, J=2.4 Hz, 1H), 7.51 (dd, J=8.9, 2.6 Hz, 1H), 7.43 (d, J=2.6 Hz, 1H), 7.08-7.01 (m, 2H), 6.85 (d, J=8.7 Hz, 1H), 4.26 (q, J=5.9 Hz, 1H), 3.81 (s, 3H), 2.54 (s, 3H), 1.84-1.63 (m, 4H), 1.54-1.41 (m, 2H), 1.36 (s, 9H), 1.01 (t, J=7.4 Hz, 3H), 0.94 (t, J=7.3 Hz, 3H). ¹³C NMR (126 MHz, CDCl₃) δ 159.22, 152.73, 145.05, 143.62, 139.01, 138.66, 131.79, 128.59, 128.28, 125.90, 125.02, 120.14, 117.02, 113.23, 112.08, 80.32, 56.23, 35.51, 34.45, 31.60, 26.57, 18.69, 14.23, 9.57, 9.28.

vvvvv. Preparation of F9

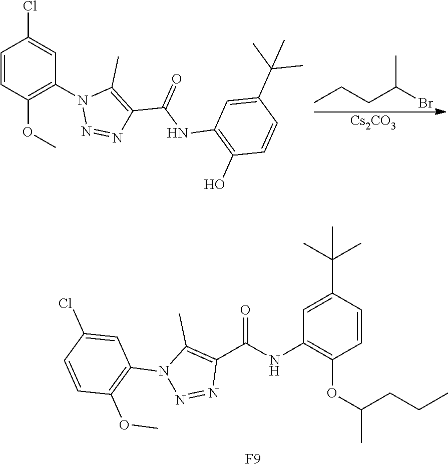

The preparation of compound F9 was prepared by reaction of N-(5-(tert-butyl)-2-hydroxyphenyl)-1-(5-chloro-2-methoxyphenyl)-5-methyl-1H-1,2,3-triazole-4-carboxamide and 2-bromopentane according to the procedure for F8 synthesis. 81.9 mg, 70% yield, white solid. ¹H NMR (500 MHz, CDCl3) δ 9.83 (s, 1H), 8.64 (d, J=2.4 Hz, 1H), 7.51 (dd, J=8.9, 2.6 Hz, 1H), 7.42 (d, J=2.6 Hz, 1H), 7.08-7.02 (m, 2H), 6.86 (d, J=8.6 Hz, 1H), 4.43 (h, J=6.1 Hz, 1H), 3.81 (s, 3H), 2.53 (s, 3H), 1.94-1.79 (m, 1H), 1.69-1.62 (m, 1H), 1.56-1.47 (m, 2H), 1.37 (d, J=6.2 Hz, 3H), 1.36 (s, 9H), 0.96 (t, J=7.3 Hz, 3H). 13C NMR (126 MHz, CDCl3) δ 159.21, 152.73, 144.72, 143.82, 139.05, 138.64, 131.81, 128.58, 128.39, 125.90, 125.00, 120.21, 117.08, 113.25, 112.40, 75.40, 56.23, 38.68, 34.46, 31.59, 19.92, 18.78, 14.14, 9.28.

wwwww. Preparation of F10

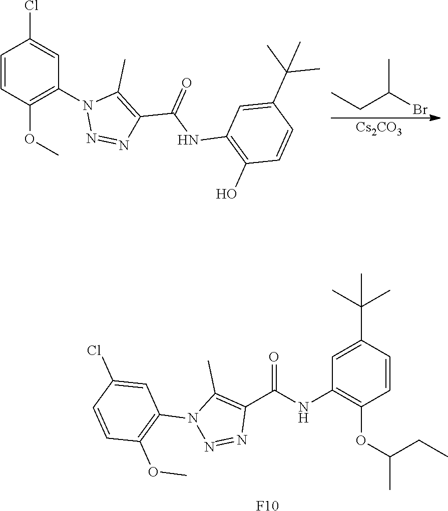

The preparation of compound F10 was prepared by reaction N-(5-(tert-butyl)-2-hydroxyphenyl)-1-(5-chloro-2-methoxyphenyl)-5-methyl-1H-1,2,3-triazole-4-carboxamide and 2-bromobutane according to the procedure for F8 synthesis. 84.6 mg, 74% yield, white solid. ¹H NMR (500 MHz, CDCl3) δ 9.84 (s, 1H), 8.64 (d, J=2.4 Hz, 1H), 7.51 (dd, J=8.9, 2.6 Hz, 1H), 7.42 (d, J=2.6 Hz, 1H), 7.08-7.01 (m, 2H), 6.86 (d, J=8.6 Hz, 1H), 4.37 (h, J=6.0 Hz, 1H), 3.81 (s, 3H), 2.53 (s, 3H), 1.87 (tt, J=13.6, 7.5 Hz, 1H), 1.79-1.69 (m, 1H), 1.38 (d, J=6.1 Hz, 3H), 1.36 (s, 9H), 1.05 (t, J=7.5 Hz, 3H). ¹³C NMR (126 MHz, CDCl3) δ 159.20, 152.72, 144.70, 143.85, 139.04, 138.64, 131.80, 128.58, 128.41, 125.90, 125.01, 120.20, 117.08, 113.24, 112.46, 76.74, 56.23, 34.46, 31.59, 29.31, 19.39, 9.76, 9.28.

xxxxx. Preparation of G1

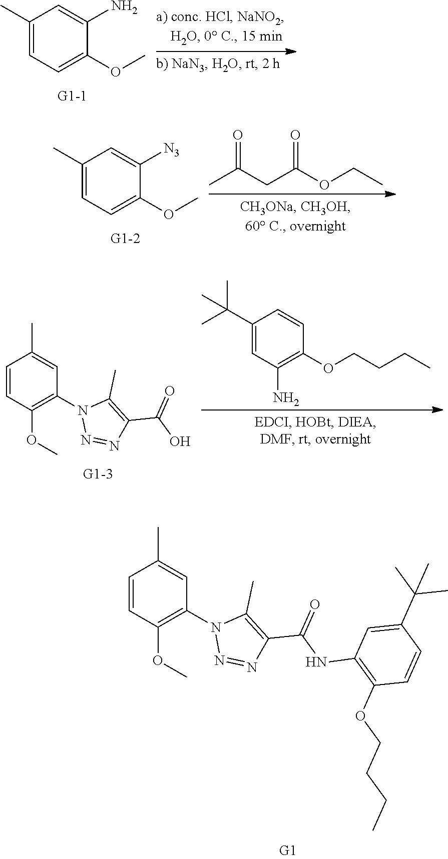

The preparation of compound G1 was prepared by 2-methoxy-5-methylaniline as start compound according to the procedure for A1 synthesis. White solid, 369.8 mg, 82% yield. ¹H NMR (400 MHz, Chloroform-d) δ 9.83 (s, 1H), 8.67 (d, J=2.4 Hz, 1H), 7.31 (dd, J=8.4, 2.3 Hz, 1H), 7.20 (d, J=2.2 Hz, 1H), 7.07 (dd, J=8.5, 2.4 Hz, 1H), 6.97 (d, J=8.5 Hz, 1H), 6.86 (d, J=8.5 Hz, 1H), 4.08 (t, J=6.4 Hz, 2H), 3.76 (s, 3H), 2.53 (s, 3H), 2.36 (s, 3H), 1.88 (dq, J=8.1, 6.5 Hz, 2H), 1.66-1.55 (m, 2H), 1.36 (s, 9H), 1.00 (t, J=7.4 Hz, 3H). ¹³C NMR (101 MHz, CDCl3) δ 159.51, 151.78, 145.72, 143.76, 138.98, 138.43, 132.33, 130.77, 128.83, 127.59, 123.91, 120.07, 117.01, 112.08, 110.59, 68.51, 55.88, 34.45, 31.60, 31.33, 20.26, 19.26, 13.89, 9.27. ESI-TOF HRMS: m/z 451.2706 (C₂₆H₃₄N₄O₃+H⁺ requires 451.2704).

yyyyy. Preparation of G2

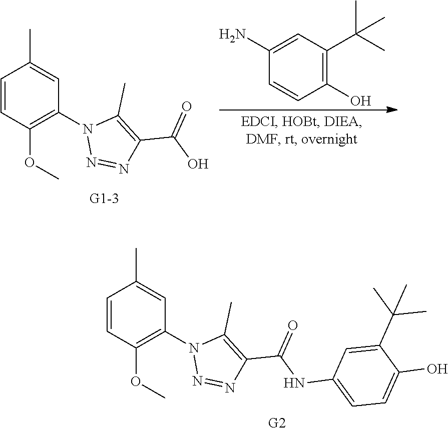 [SMILES: COc1ccc(C)cc1-n1nnc(C(=O)O)c1C ; CC(C)(C)c1cc(N)ccc1O ; COc1ccc(C)cc1-n1nnc(C(=O)Nc2ccc(O)c(C(C)(C)C)c2)c1C ; "G2"]

The preparation of compound G2 was prepared by reaction of 1-(2-methoxy-5-methylphenyl)-5-methyl-1H-1,2,3-triazole-4-carboxylic acid and 4-amino-2-(tert-butyl)phenol according to the procedure for A1 synthesis. White solid, 1.32 g, 67% yield. ¹H NMR (400 MHz, Chloroform-d) δ 8.96 (s, 1H), 7.53 (dd, J=8.5, 2.6 Hz, 1H), 7.44 (d, J=2.6 Hz, 1H), 7.33 (ddd, J=8.5, 2.2, 0.8 Hz, 1H), 7.19 (dd, J=2.2, 0.8 Hz, 1H), 6.99 (d, J=8.5 Hz, 1H), 6.69 (d, J=8.5 Hz, 1H), 3.78 (s, 3H), 2.50 (s, 3H), 2.37 (d, J=0.7 Hz, 3H), 1.43 (s, 9H). ¹³C NMR (101 MHz, CDCl3) δ 159.36, 151.80, 151.14, 139.18, 137.96, 136.79, 132.42, 130.84, 130.57, 128.84, 123.80, 119.70, 119.14, 116.80, 112.08, 55.90, 34.69, 29.52, 20.27, 9.21. ESI-TOF HRMS: m/z 395.2076 (C₂₂H₂₆N₄O₃+H⁺ requires 395.2078).

zzzzz. Preparation of G3

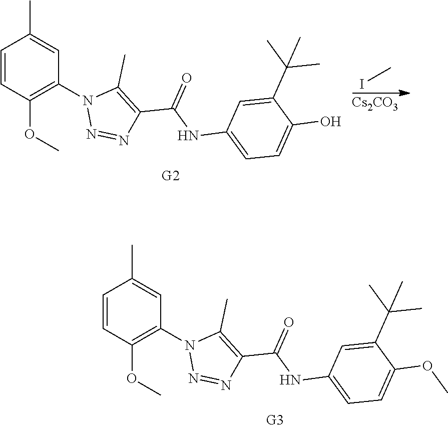

The preparation of compound G3 was prepared by reaction of N-(3-(tert-butyl)-4-hydroxyphenyl)-1-(2-methoxy-5-methylphenyl)-5-methyl-1H-1,2,3-triazole-4-carboxamide and iodomethane according to the procedure for F5 synthesis. White solid, 140.8 mg, 86% yield. ¹H NMR (400 MHz, Chloroform-d) δ 8.97 (s, 1H), 7.67 (dd, J=8.7, 2.6 Hz, 1H), 7.45 (d, J=2.7 Hz, 1H), 7.33 (ddd, J=8.5, 2.3, 0.8 Hz, 1H), 7.19 (dd, J=2.2, 0.8 Hz, 1H), 6.99 (d, J=8.5 Hz, 1H), 6.88 (d, J=8.7 Hz, 1H), 3.85 (s, 3H), 3.78 (s, 3H), 2.50 (s, 3H), 2.37 (d, J=0.7 Hz, 3H), 1.40 (s, 9H). ¹³C NMR (101 MHz, CDCl3) δ 159.29, 155.41, 151.81, 139.13, 138.95, 138.02, 132.40, 130.83, 130.48, 128.84, 123.83, 119.29, 118.76, 112.08, 111.94, 55.90, 55.36, 34.94, 29.67, 20.27, 9.21. ESI-TOF HRMS: m/z 395.2076 (C₂₂H₂₆N₄O₃+H⁺ requires 395.2078). ESI-TOF HRMS: m/z 409.2231 (C₂₃H₂₈N₄O₃+H⁺ requires 409.2234).

aaaaaa. Preparation of G4

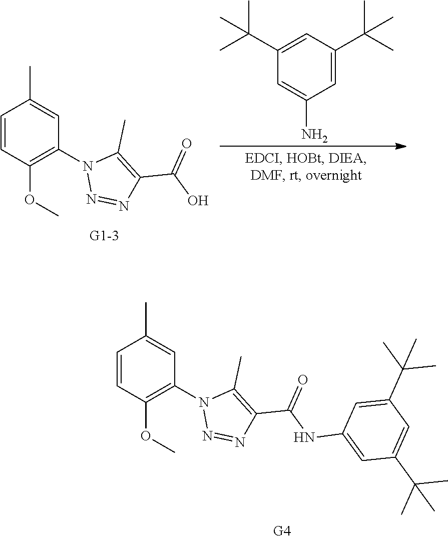 [SMILES: COc1ccc(C)cc1-n1nnc(C(=O)O)c1C ; CC(C)(C)c1cc(N)cc(C(C)(C)C)c1 ; COc1ccc(C)cc1-n1nnc(C(=O)Nc2cc(C(C)(C)C)cc(C(C)(C)C)c2)c1C ; "G4"]

The preparation of compound G4 was prepared by reaction of 1-(2-methoxy-5-methylphenyl)-5-methyl-1H-1,2,3-triazole-4-carboxylic acid and 3,5-di-tert-butylaniline according to the procedure for A1 synthesis. White solid, 165.3 mg, 76% yield. ¹H NMR (400 MHz, Chloroform-d) δ 9.10 (s, 1H), 7.60 (d, J=1.7 Hz, 2H), 7.33 (ddd, J=8.5, 2.3, 0.8 Hz, 1H), 7.24-7.19 (m, 2H), 6.99 (d, J=8.5 Hz, 1H), 3.78 (s, 3H), 2.52 (s, 3H), 2.37 (d, J=0.9 Hz, 3H), 1.36 (s, 18H). ¹³C NMR (101 MHz, CDCl3) δ 159.40, 151.80, 151.69, 139.21, 138.05, 137.30, 132.43, 130.83, 128.84, 123.81, 118.39, 114.37, 112.09, 55.89, 35.00, 31.47, 20.27, 9.27. ESI-TOF HRMS: m/z 435.2740 (C₂₆H₃₄N₄O₂+H^{|} requires 435.2755).

bbbbbb. Preparation of G5

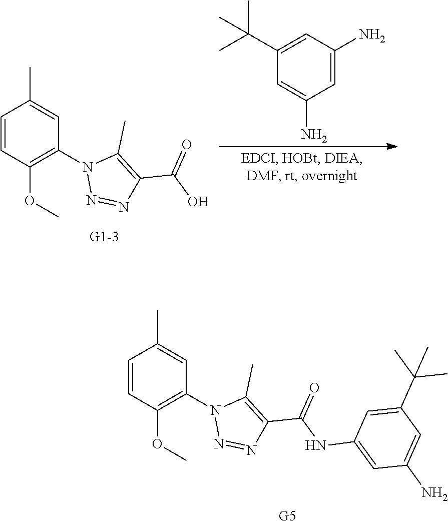 [SMILES: COc1ccc(C)cc1-n1nnc(C(=O)O)c1C ; CC(C)(C)c1cc(N)cc(N)c1 ; COc1ccc(C)cc1-n1nnc(C(=O)Nc2cc(N)cc(C(C)(C)C)c2)c1C ; "G5"]

The preparation of compound G5 was prepared by reaction of 1-(2-methoxy-5-methylphenyl)-5-methyl-1H-1,2,3-triazole-4-carboxylic acid and 5-(tert-butyl)benzene-1,3-diamine according to the procedure for A1 synthesis. ¹H NMR (500 MHz, CDCl₃) δ 9.03 (s, 1H), 7.37 (t, J=2.0 Hz, 1H), 7.36-7.31 (m, 1H), 7.19 (d, J=2.4 Hz, 1H), 7.05 (t, J=1.8 Hz, 1H), 6.98 (d, J=8.5 Hz, 1H), 6.67 (t, J=1.9 Hz, 1H), 3.78 (s, 3H), 2.49 (s, 3H), 2.37 (s, 3H), 1.31 (s, 9H). ¹³C NMR (126 MHz, CDCl₃) δ 159.44, 153.57, 151.78, 143.98, 139.37, 138.55, 137.88, 132.44, 130.83, 128.84, 123.75, 112.06, 109.90, 109.20, 105.41, 55.90, 34.79, 31.25, 20.28, 9.24. ESI-TOF HRMS: m/z 394.2238 (C₂₂H₂₇N₅O₂+H⁺ requires 394.2238).

cccccc. Preparation of G6

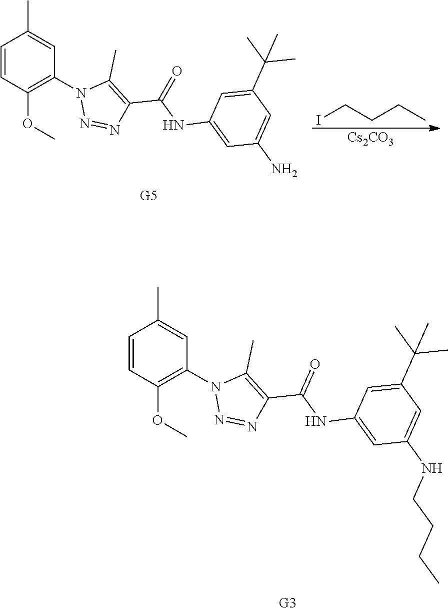

The preparation of compound G6 was prepared by reaction of N-(3-amino-5-(tert-butyl)phenyl)-1-(2-methoxy-5-methylphenyl)-5-methyl-1H-1,2,3-triazole-4-carboxamide and 1-iodobutane according to the procedure for D2 synthesis. White solid, 142.8 mg, 79% yield. ¹H NMR (400 MHz, Chloroform-d) δ 9.01 (s, 1H), 7.33 (ddd, J=8.5, 2.3, 0.8 Hz, 1H), 7.19 (dd, J=2.2, 0.8 Hz, 1H), 7.10 (t, J=2.0 Hz, 1H), 6.99 (d, J=8.5 Hz, 1H), 6.92 (t, J=1.7 Hz, 1H), 6.44 (t, J=1.9 Hz, 1H), 3.78 (s, 3H), 3.16 (t, J=7.1 Hz, 2H), 2.50 (s, 3H), 2.37 (s, 3H), 1.68-1.56 (m, 2H), 1.50-1.39 (m, 2H), 1.31 (s, 9H), 0.97 (t, J=7.3 Hz, 3H). ¹³C NMR (101 MHz, CDCl3) δ 159.35, 153.02, 151.80, 148.92, 139.19, 138.60, 138.08, 132.41, 130.82, 128.84, 123.82, 112.08, 106.53, 106.29, 101.34, 55.89, 43.86, 34.77, 31.73, 31.31, 20.33, 20.27, 13.96, 9.24. ESI-TOF HRMS: m/z 450.2855 (C₂₆H₃₅N₅O₂+H⁺ requires 450.2864).

dddddd. Preparation of G7

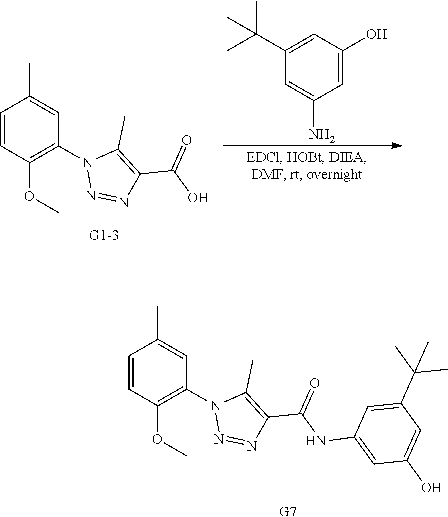 [SMILES: COc1ccc(C)cc1-n1nnc(C(=O)O)c1C ; CC(C)(C)c1cc(N)cc(O)c1 ; COc1ccc(C)cc1-n1nnc(C(=O)Nc2cc(O)cc(C(C)(C)C)c2)c1C ; "G7"]

The preparation of compound G7 was prepared by reaction of N-(3-amino-5-(tert-butyl)phenyl)-1-(2-methoxy-5-methylphenyl)-5-methyl-1H-1,2,3-triazole-4-carboxamide and 3-amino-5-(tert-butyl)phenol according to the procedure for A1 synthesis. White solid, 788.6 mg, 67% yield. ¹H NMR (400 MHz, Chloroform-d) δ 9.14 (s, 1H), 7.58-7.53 (m, 1H), 7.34 (ddd, J=8.4, 2.3, 0.8 Hz, 1H), 7.20 (dd, J=2.2, 0.8 Hz, 1H), 7.03-6.98 (m, 2H), 6.70 (dd, J=2.3, 1.6 Hz, 1H), 3.79 (s, 3H), 2.51 (s, 3H), 2.38 (s, 3H), 1.30 (s, 9H). 13C NMR (101 MHz, CDCl3) δ 159.68, 156.39, 153.83, 151.80, 139.59, 138.25, 137.78, 132.52, 130.86, 128.82, 123.67, 112.10, 109.13, 109.03, 104.62, 55.91, 34.81, 31.22, 20.27, 9.27. ESI-TOF HRMS: m/z 395.2082 (C₂₂H₂₆N₄O₃+H⁺ requires 395.2078).

eeeeee. Preparation of G8

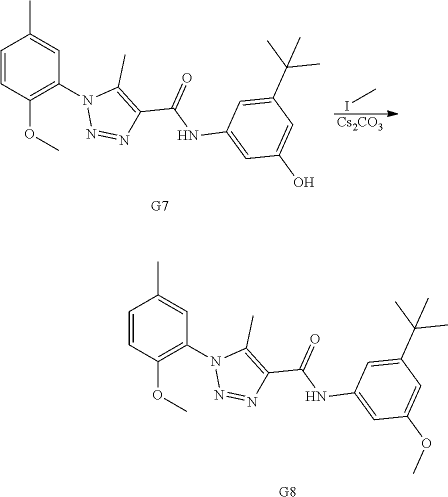

The preparation of compound G8 was prepared by reaction of N-(3-(tert-butyl)-5-hydroxyphenyl)-1-(2-methoxy-5-methylphenyl)-5-methyl-1H-1,2,3-triazole-4-carboxamide and iodomethane according to the procedure for F5 synthesis. White solid, 149.6 mg, 92% yield. ¹H NMR (400 MHz, Chloroform-d) δ 9.10 (s, 1H), 7.42 (t, J=2.1 Hz, 1H), 7.34 (ddd, J=8.5, 2.3, 0.8 Hz, 1H), 7.20 (dd, J=2.2, 0.8 Hz, 1H), 7.16 (t, J=1.7 Hz, 1H), 6.99 (d, J=8.5 Hz, 1H), 6.75 (dd, J=2.4, 1.6 Hz, 1H), 3.85 (s, 3H), 3.79 (s, 3H), 2.51 (s, 3H), 2.38 (s, 3H), 1.33 (s, 9H). ¹³C NMR (101 MHz, CDCl3) δ 159.97, 159.44, 153.48, 151.79, 139.34, 138.65, 137.90, 132.46, 130.86, 128.82, 123.75, 112.09, 109.37, 108.59, 101.76, 55.91, 55.37, 34.90, 31.27, 20.27, 9.25. ESI-TOF HRMS: m/z 409.2234 (C₂₃H₂₈N₄O₃+H⁺ requires 409.2234).

ffffff. Preparation of G9

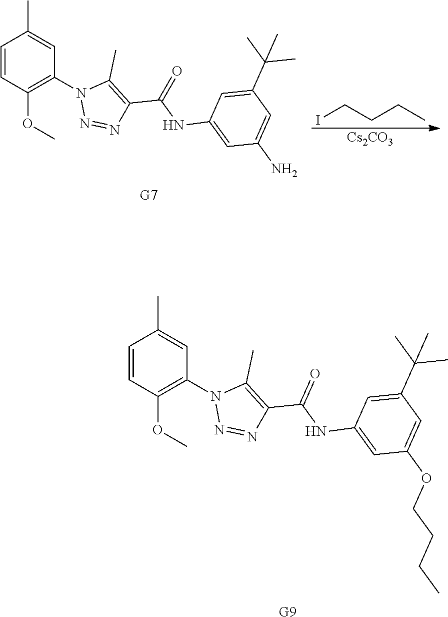

The preparation of compound G9 was prepared by reaction of N-(3-(tert-butyl)-5-hydroxyphenyl)-1-(2-methoxy-5-methylphenyl)-5-methyl-1H-1,2,3-triazole-4-carboxamide and 1-iodobutane according to the procedure for F5 synthesis. White solid, 144.3 mg, 80% yield. ¹H NMR (400 MHz, Chloroform-d) δ 9.09 (s, 1H), 7.41 (t, J=2.1 Hz, 1H), 7.33 (ddt, J=8.4, 2.2, 0.8 Hz, 1H), 7.20 (d, J=2.2 Hz, 1H), 7.14 (t, J=1.7 Hz, 1H), 6.99 (d, J=8.5 Hz, 1H), 6.74 (dd, J=2.3, 1.7 Hz, 1H), 4.01 (t, J=6.5 Hz, 2H), 3.78 (d, J=0.9 Hz, 3H), 2.51 (s, 3H), 2.37 (s, 3H), 1.84-1.69 (m, 2H), 1.59-1.45 (m, 2H), 1.33 (s, 9H), 0.99 (t, J=7.4 Hz, 3H). ¹³C NMR (101 MHz, CDCl3) δ 159.56, 159.39, 153.39, 151.79, 139.32, 138.57, 137.91, 132.46, 130.85, 128.82, 123.75, 112.09, 109.18, 109.06, 102.36, 67.72, 55.90, 34.89, 31.46, 31.28, 20.27, 19.31, 13.92, 9.25. ESI-TOF HRMS: m/z 451.2707 (C₂₆H₃₄N₄O₃+H⁺ requires 451.2704).

gggggg. Preparation of G10

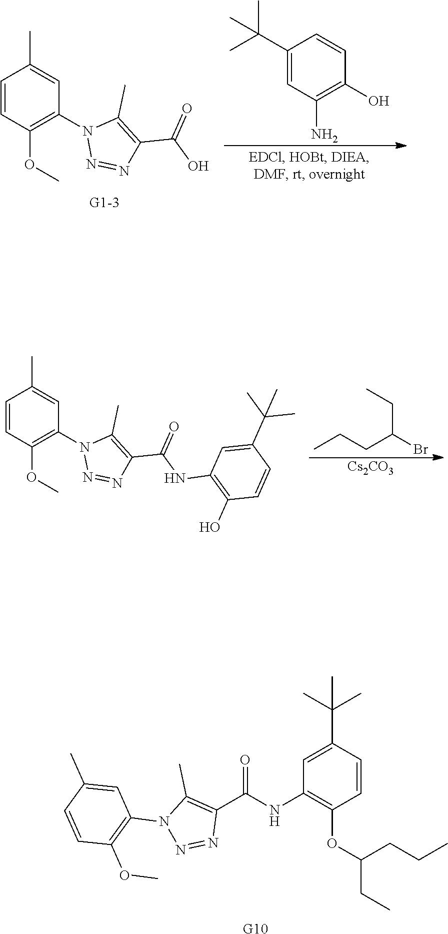

To a solution of 1-(2-methoxy-5-methylphenyl)-5-methyl-1H-1,2,3-triazole-4-carboxylic acid (299 mg, 1.210 mmol) in DMF (5 mL) was added 2-amino-4-(tert-butyl)phenol (200 mg, 1.210 mmol). HOBt, 80% (307 mg, 1.816 mmol) and EDCI (348 mg, 1.816 mmol), followed by N-ethyl-N-isopropylpropan-2-amine (626 mg, 4.84 mmol). The suspension stirred at room temperature for overnight. The reaction mixture was diluted with water (50 mL) and extracted with EtOAc (50 mL×2). The EtOAc layer was washed with water, dried with anhydrous Na₂SO₄ and concentrated. The residue was purified by silica gel chromatography (0% to 100% acetonitrile in water) to give product as white solid (300.8 mg, 63% yield). ¹H NMR (500 MHz, DMSO-d6) δ 10.02 (s, 1H), 9.61 (s, 1H), 8.33 (d, J=2.4 Hz, 1H), 7.45 (ddd, J=8.5, 2.3, 0.8 Hz, 1H), 7.32 (dd, J=2.1, 0.8 Hz, 1H), 7.24 (d, J=8.6 Hz, 1H), 6.98 (dd, J=8.5, 2.4 Hz, 1H), 6.86 (d, J=8.4 Hz, 1H), 3.78 (s, 3H), 2.39 (s, 3H), 2.34 (s, 3H), 1.27 (s, 9H). ¹³C NMR (126 MHz, DMSO) δ 157.91, 150.85, 143.58, 140.93, 138.17, 136.74, 132.04, 129.63, 128.02, 125.07, 122.43, 120.08, 116.22, 113.70, 112.16, 55.42, 33.35, 30.83, 19.13, 8.16.

To a solution of N-(5-(tert-butyl)-2-hydroxyphenyl)-1-(2-methoxy-5-methylphenyl)-5-methyl-1H-1,2,3-triazole-4-carboxamide (100 mg, 0.254 mmol) in acetone 5 mL at rt was added Cs₂CO₃ (165 mg, 0.507 mmol) and 3-bromohexane (50.2 mg, 0.304 mmol). The suspension was stirred at 60° C. for overnight. The reaction mixture was then diluted by water (25 mL) and extracted with EtOAc (25 mL×2). The EtOAc layer was washed with water, dried with anhydrous Na₂SO₄ and concentrated. The residue was purified by silica gel chromatography (0% to 100% EA in hexane) to give product as a white solid (97.8 mg, 81% yield). ¹H NMR (500 MHz, CDCl3) δ 9.89 (s, 1H), 8.66 (d, J=2.4 Hz, 1H), 7.32 (ddd, J=8.5, 2.3, 0.8 Hz, 1H), 7.22-7.19 (m, 1H), 7.05 (dd, J=8.6, 2.5 Hz, 1H), 6.98 (d, J=8.5 Hz, 1H), 6.84 (d, J=8.6 Hz, 1H), 4.27 (p, J=5.8 Hz, 1H), 3.78 (s, 3H). 2.52 (s, 3H), 2.37 (s, 3H), 1.82-1.63 (m, 4H), 1.53-1.42 (m, 2H), 1.36 (s, 9H), 1.01 (t, J=7.4 Hz, 3H), 0.94 (t, J=7.4 Hz, 3H). ¹³C NMR (126 MHz, CDCl3) δ 159.51, 151.77, 145.04, 143.59, 138.94, 138.50, 132.27, 130.77, 128.87, 128.40, 123.95, 120.01, 117.01, 112.05, 112.02, 80.29, 55.89, 35.51, 34.45, 31.60, 26.57, 20.28, 18.69, 14.24, 9.58, 9.30.

hhhhhh. Preparation of G11

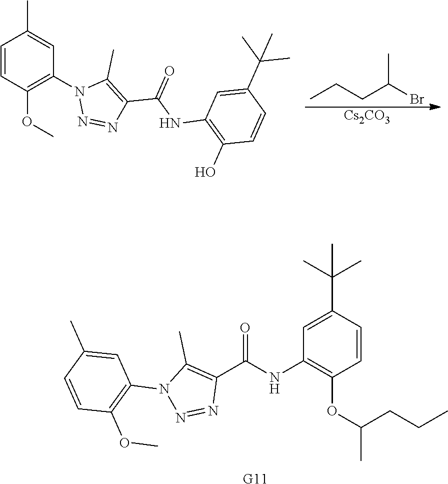

The preparation of compound G10 was prepared by reaction of N-(5-(tert-butyl)-2-hydroxyphenyl)-1-(2-methoxy-5-methylphenyl)-5-methyl-1H-1,2,3-triazole-4-carboxamide and 2-bromopentane according to the procedure for G10 synthesis. 89.8 mg, 75% yield, white solid. ¹H NMR (500 MHz, CDCl3) δ 9.85 (s, 1H), 8.66 (d, J=2.4 Hz, 1H), 7.32 (ddd, J=8.5, 2.2, 0.9 Hz, 1H), 7.20 (d, J=2.1 Hz, 1H), 7.05 (dd, J=8.5, 2.4 Hz, 1H), 6.98 (d, J=8.5 Hz, 1H), 6.86 (d, J=8.5 Hz, 1H), 4.43 (h, J=6.1 Hz, 1H), 3.78 (s, 3H), 2.52 (s, 3H), 2.37 (s, 3H), 1.93-1.83 (m, 1H), 1.68-1.63 (m, 1H), 1.58-1.45 (m, 2H), 1.37 (d, J=6.1 Hz, 3H), 1.36 (s, 9H), 0.96 (t, J=7.4 Hz, 3H). ¹³C NMR (126 MHz, CDCl3) δ 159.50, 151.78, 144.72, 143.79, 138.97, 138.48, 132.29, 130.78, 128.86, 128.51, 123.93, 120.07, 117.07, 112.39, 112.04, 75.38, 55.89, 38.68, 34.46, 31.60, 20.28, 19.91, 18.79, 14.15, 9.30.

iiiiii. Preparation of G12

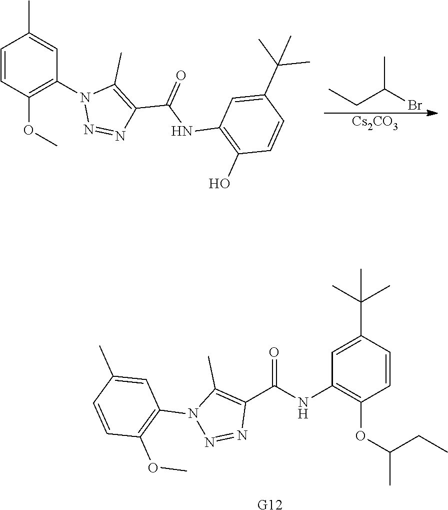

The preparation of compound G12 was prepared by reaction N-(5-(tert-butyl)-2-hydroxyphenyl)-1-(2-methoxy-5-methylphenyl)-5-methyl-1H-1,2,3-triazole-4-carboxamide and 2-bromobutane according to the procedure for G10 synthesis. 99.8 mg, 88% yield, white solid. ¹H NMR (500 MHz, CDCl3) δ 9.87 (s, 1H), 8.66 (d, J=2.4 Hz, 1H), 7.32 (dd, J=8.4, 2.2 Hz, 1H), 7.20 (d, J=2.2 Hz, 1H), 7.05 (dd, J=8.6, 2.4 Hz, 1H), 6.98 (d, J=8.5 Hz, 1H), 6.86 (d, J=8.6 Hz, 1H), 4.37 (h, J=6.0 Hz, 1H), 3.78 (s, 3H), 2.52 (s, 3H), 2.37 (s, 3H), 1.94-1.82 (m, 1H), 1.79-1.69 (m, 1H), 1.38 (d, J=6.1 Hz, 3H), 1.36 (s, 9H), 1.05 (t, J=7.5 Hz, 3H). ¹³C NMR (126 MHz, CDCl3) δ 159.49, 151.77, 144.70, 143.83, 138.97, 138.48, 132.29, 130.77, 128.86, 128.53, 123.94, 120.07, 117.07, 112.46, 112.04, 76.73, 55.89, 34.46, 31.60, 29.31, 20.28, 19.39, 9.78, 9.30.

jjjjjj. Preparation of H1

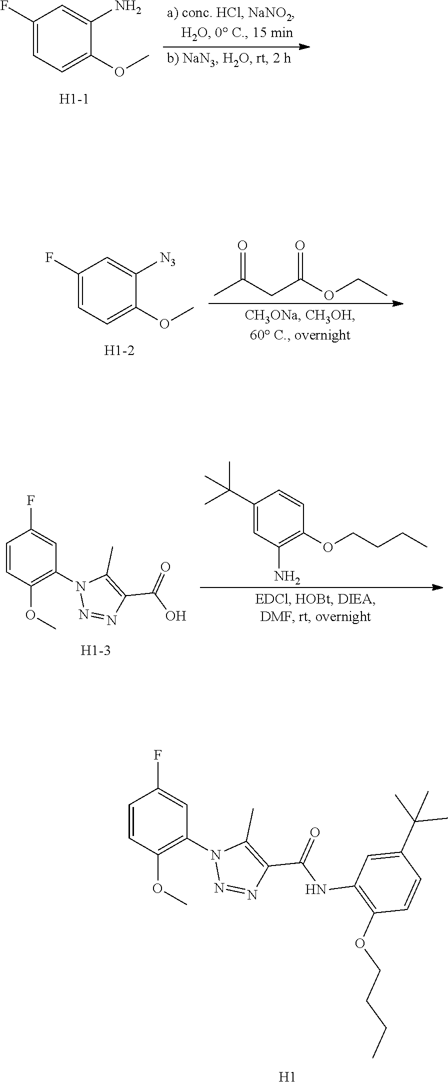

The preparation of compound H1 was prepared by 5-fluoro-2-methoxyaniline as start compound according to the procedure for A1 synthesis. White solid, 339.5 mg, 75% yield (final step). ¹H NMR (400 MHz, Chloroform-d) δ 9.81 (s, 1H), 8.65 (d, J=2.4 Hz, 1H), 7.28-7.21 (m, 1H), 7.18 (dd, J=7.8, 3.1 Hz, 1H), 7.09-7.01 (m, 2H), 6.86 (d, J=8.6 Hz, 1H), 4.08 (t, J=6.4 Hz, 2H), 3.78 (s, 3H), 2.54 (s, 3H), 1.87 (dq, J=7.9, 6.4 Hz, 2H), 1.64-1.53 (m, 2H), 1.36 (s, 9H), 1.00 (t, J=7.4 Hz, 3H). ¹³C NMR (101 MHz, CDCl3) δ 159.24, 157.43, 155.02, 150.43, 150.40, 145.71, 143.78, 139.09, 138.57, 127.47, 124.57, 124.47, 120.19, 120.15, 118.47, 118.25, 117.01, 116.02, 115.77, 113.12, 113.04, 110.62, 68.51, 56.36, 34.44, 31.59, 31.31, 19.25, 13.87, 9.25. ESI-TOF HRMS: m/z 455.2453 (C₂₅H₃₁FN₄O₃+H⁺ requires 455.2453).

kkkkkk. Preparation of H2

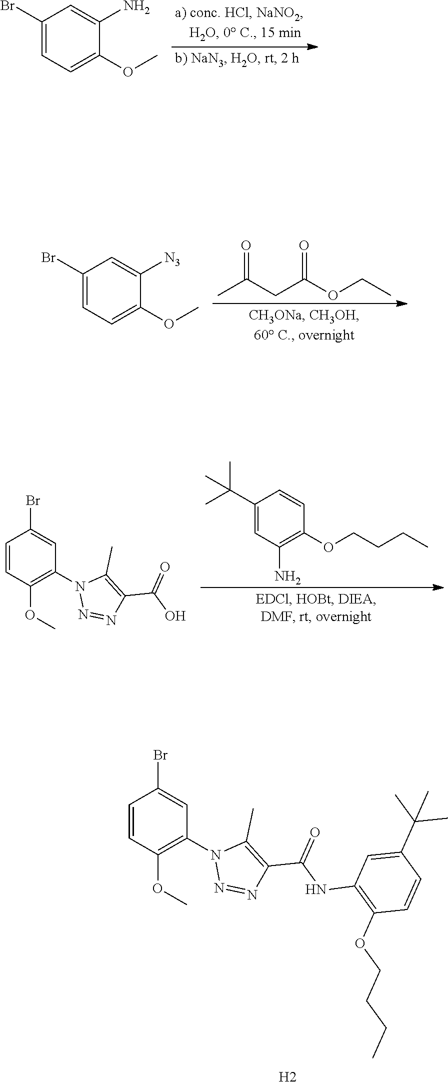

The preparation of compound H2 was prepared by 5-bromo-2-methoxyaniline as start compound according to the procedure for A1 synthesis. White solid, 436.8 mg, 85% yield (final step). ¹H NMR (400 MHz, Chloroform-d) δ 9.80 (s, 1H), 8.64 (d, J=2.4 Hz, 1H), 7.64 (dd, J=8.9, 2.5 Hz, 1H), 7.56 (d, J=2.4 Hz, 1H), 7.07 (dd, J=8.5, 2.4 Hz, 1H), 6.99 (d, J=8.9 Hz, 1H), 6.86 (d, J=8.6 Hz, 1H), 4.08 (t, J=6.4 Hz, 2H), 3.80 (s, 3H), 2.54 (s, 3H), 1.88 (dq, J=8.4, 6.5 Hz, 2H), 1.66-1.54 (m, 2H), 1.36 (s, 9H), 1.01 (t, J=7.4 Hz, 3H). ¹³C NMR (101 MHz, CDCl3) δ 159.22, 153.24, 145.72, 143.79, 139.06, 138.58, 134.76, 131.36, 127.46, 125.32, 120.18, 117.04, 113.71, 112.62, 110.60, 68.52, 56.18, 34.45, 31.59, 31.32, 19.25, 13.88, 9.26. ESI-TOF HRMS: m/z 515.1662 (C₂₅H₃₁BrN₄O₃+H⁺ requires 515.1653).

llllll. Preparation of H3

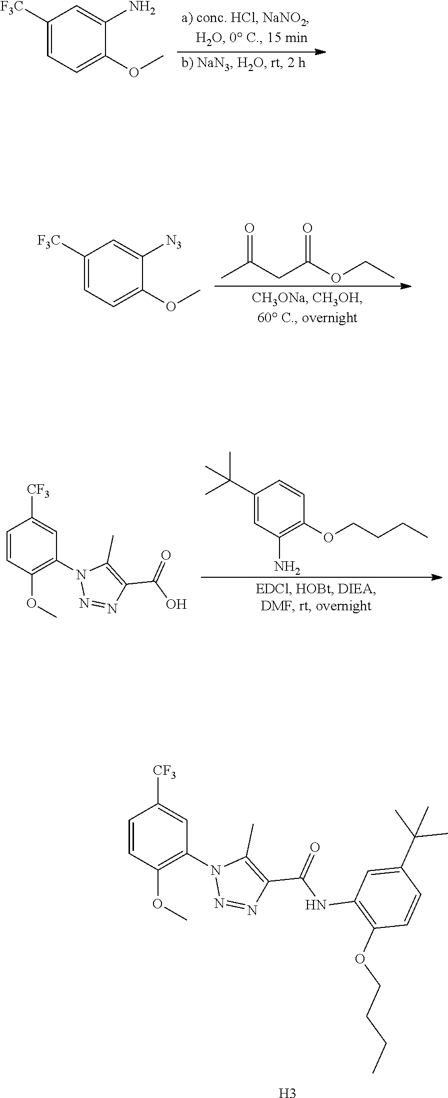

The preparation of compound H3 was prepared by 2-methoxy-5-(trifluoromethyl)aniline as start compound according to the procedure for A1 synthesis. White solid, 357.2 mg, 71% yield (final step). ¹H NMR (400 MHz, Chloroform-d) δ 9.82 (s, 1H), 8.65 (d, J=2.4 Hz, 1H), 7.81 (dd, J=8.8, 2.3 Hz, 1H), 7.72 (d, J=2.3 Hz, 1H), 7.20 (d, J=8.8 Hz, 1H), 7.08 (dd, J=8.5, 2.4 Hz, 1H), 6.87 (d, J=8.5 Hz, 1H), 4.09 (t, J=6.5 Hz, 2H), 3.88 (s, 3H), 2.55 (s, 3H), 1.88 (dq, J=8.3, 6.5 Hz, 2H). 1.66-1.55 (m, 2H), 1.36 (s, 9H), 1.01 (t, J=7.4 Hz, 3H). ¹³C NMR (101 MHz, CDCl3) δ 159.15, 156.43, 145.73, 143.80, 139.13, 138.71, 129.40, 129.36, 129.32, 129.28, 127.51, 127.43, 126.18, 126.14, 126.10, 126.07, 124.81, 124.51, 124.18, 123.84, 123.50, 123.17, 122.11, 120.25, 117.02, 112.42, 110.62, 68.52, 56.34, 34.44, 31.58, 31.31, 19.25, 13.86, 9.25. ESI-TOF HRMS: m/z 505.2417 (C₂₆H₃₁F₃N₄O₃+H⁺ requires 505.2421).

mmmmmm. Preparation of H4

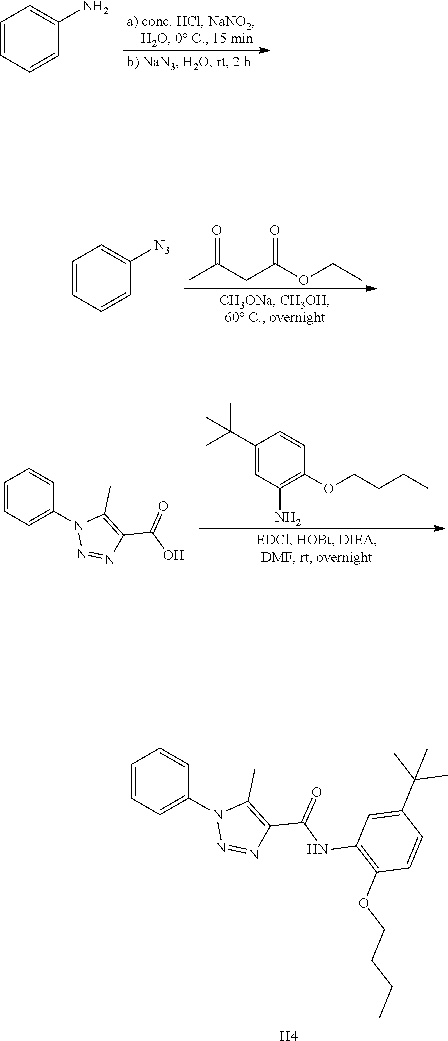

The preparation of compound H4 was prepared by aniline as start compound according to the procedure for A1 synthesis. White solid, 321.8 mg, 79% yield (final step). ¹H NMR (400 MHz, Chloroform-d) δ 9.82 (s, 1H), 8.64 (d, J=2.4 Hz, 1H), 7.62-7.55 (m, 3H), 7.52-7.48 (m, 2H), 7.08 (dd, J=8.5, 2.4 Hz, 1H), 6.86 (d, J=8.5 Hz, 1H), 4.09 (t, J=6.5 Hz, 2H), 2.70 (s, 3H), 1.89 (dq, J=8.3, 6.5 Hz, 2H), 1.65-1.56 (m, 2H), 1.36 (s, 9H), 1.01 (t, J=7.4 Hz, 3H). 13C NMR (101 MHz, CDCl3) δ 159.21, 145.73, 143.81, 139.24, 136.93, 135.67, 129.96, 129.67, 127.43, 125.29, 120.23, 117.05, 110.62, 68.53, 34.45, 31.59, 31.30, 19.24, 13.87, 9.91. ESI-TOF HRMS: m/z 407.2436 (C₂₄H₃₀N₄O₂+H⁺ requires 407.2442).

nnnnnn. Preparation of H5

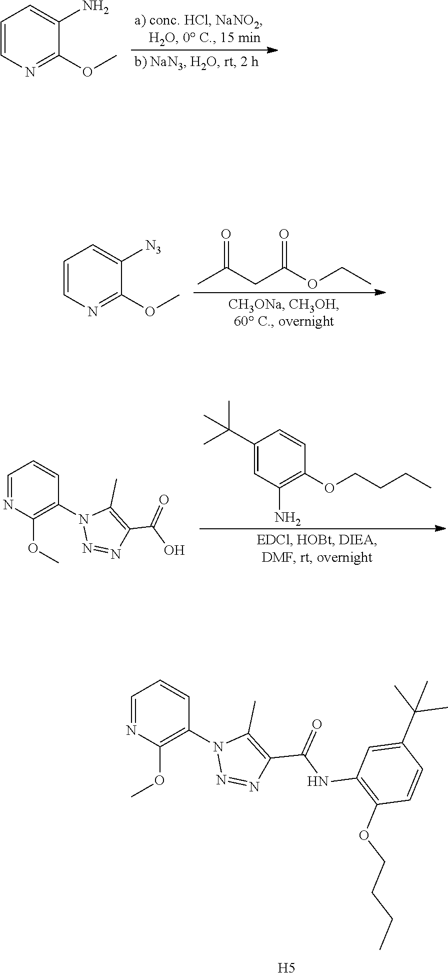

The preparation of compound H5 was prepared by 2-methoxypyridin-3-amine as start compound according to the procedure for A1 synthesis. White solid, 355.75 mg, 81% yield (final step). ¹H NMR (400 MHz, Chloroform-d) δ 9.80 (s, 1H), 8.64 (d, J=2.4 Hz, 1H), 8.38 (dd, J=5.0, 1.8 Hz, 1H), 7.75 (dd, J=7.6, 1.8 Hz, 1H), 7.12 (dd, J=7.6, 5.0 Hz, 1H), 7.07 (dd, J=8.5, 2.4 Hz, 1H), 6.86 (d, J=8.6 Hz, 1H), 4.08 (t, J=6.4 Hz, 2H), 3.98 (s, 3H), 2.56 (s, 3H), 1.94-1.81 (m, 2H), 1.67-1.50 (m, 2H), 1.36 (s, 9H), 1.00 (t, J=7.4 Hz, 3H). ¹³C NMR (101 MHz, CDCl3) δ 159.16, 158.21, 149.37, 145.71, 143.80, 138.98, 138.76, 137.06, 127.44, 120.19, 119.35, 117.10, 117.05, 110.58, 68.50, 54.14, 34.45, 31.58, 31.31, 19.24, 13.87, 9.30. ESI-TOF HRMS: m/z 438.2503 (C₂₄H₃₁N₅O₃+H⁺ requires 438.2500).

oooooo. Preparation of H6

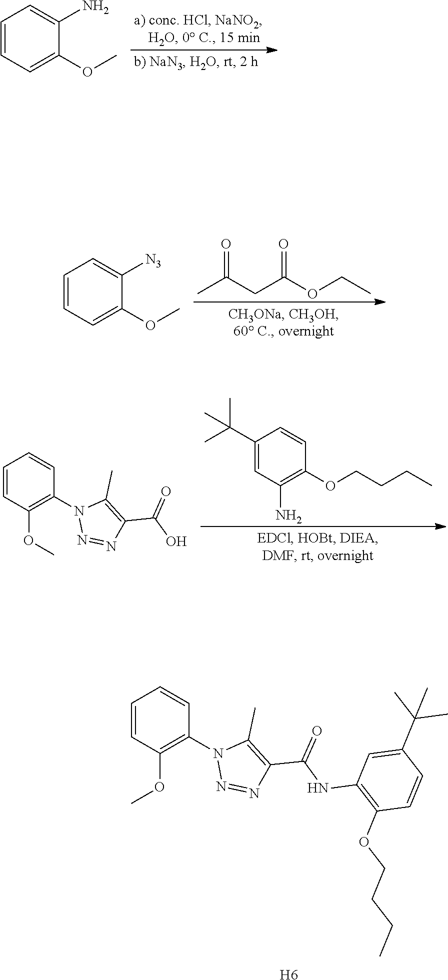

The preparation of compound H6 was prepared by 2-methoxyaniline as start compound according to the procedure for A1 synthesis. White solid, 352.2 mg, 81% yield (final step). ¹H NMR (400 MHz, Chloroform-d) δ 9.84 (s, 1H), 8.67 (d, J=2.4 Hz, 1H), 7.52 (ddd, J=8.3, 7.6, 1.7 Hz, 1H), 7.38 (dd, J=7.8, 1.7 Hz, 1H), 7.18-7.04 (m, 3H), 6.86 (d, J=8.6 Hz, 1H), 4.08 (t, J=6.4 Hz, 2H), 3.80 (s, 3H), 2.53 (s, 3H), 1.92-1.82 (m, 2H), 1.67-1.54 (m, 2H), 1.37 (s, 9H), 1.01 (t, J=7.4 Hz, 3H). ¹³C NMR (101 MHz, CDCl3) δ 159.47, 154.02, 145.72, 143.76, 139.02, 138.47, 132.02, 128.49, 127.57, 124.27, 121.04, 120.10, 117.02, 112.19, 110.59, 68.51, 55.84, 34.45, 31.61, 31.33, 19.26, 13.89, 9.27. ESI-TOF HRMS: m/z 437.2547 (C₂₅H₃₂N₄O₃+H⁺ requires 437.2547).

pppppp. Preparation of H7

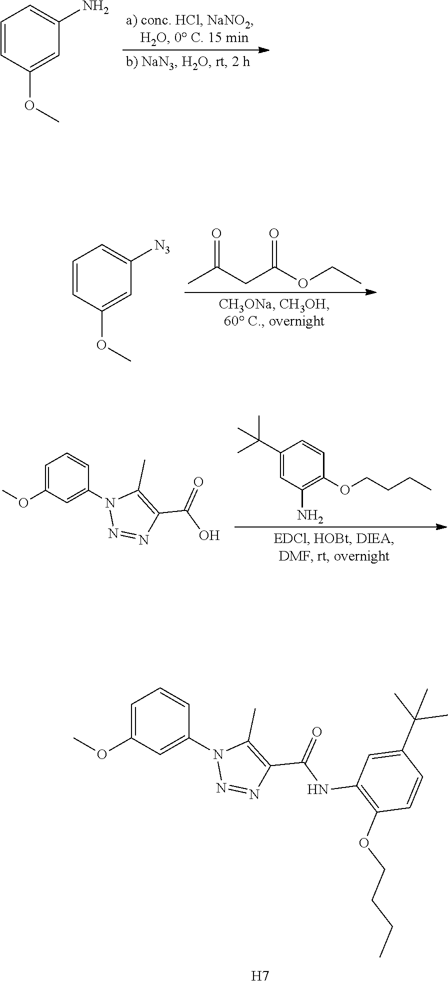

The preparation of compound H7 was prepared by 3-methoxyaniline as start compound according to the procedure for A1 synthesis. White solid, 337.6 mg, 77% yield (final step). H NMR (400 MHz, Chloroform-d) δ 9.81 (s, 1H), 8.63 (d, J=2.4 Hz, 1H), 7.50-7.45 (m, 1H), 7.12-7.03 (m, 4H), 6.86 (d, J=8.5 Hz, 1H), 4.09 (t, J=6.5 Hz, 2H), 3.88 (s, 3H), 2.71 (s, 3H), 1.88 (dq, J=8.4, 6.5 Hz, 2H), 1.67-1.53 (m, 2H), 1.36 (s, 9H), 1.01 (t, J=7.4 Hz, 3H). ¹³C NMR (101 MHz, CDCl3) δ 160.46, 159.19, 145.72, 143.81, 139.22, 136.95, 136.61, 130.35, 127.43, 120.22, 117.27, 117.04, 115.81, 111.09, 110.60, 68.52, 55.66, 34.45, 31.59, 31.30, 19.24, 13.87, 9.95. ESI-TOF HRMS: m/z 437.2541 (C₂₅H₃₂N₄O₃+H⁺ requires 437.2547).

qqqqqq. Preparation of H8

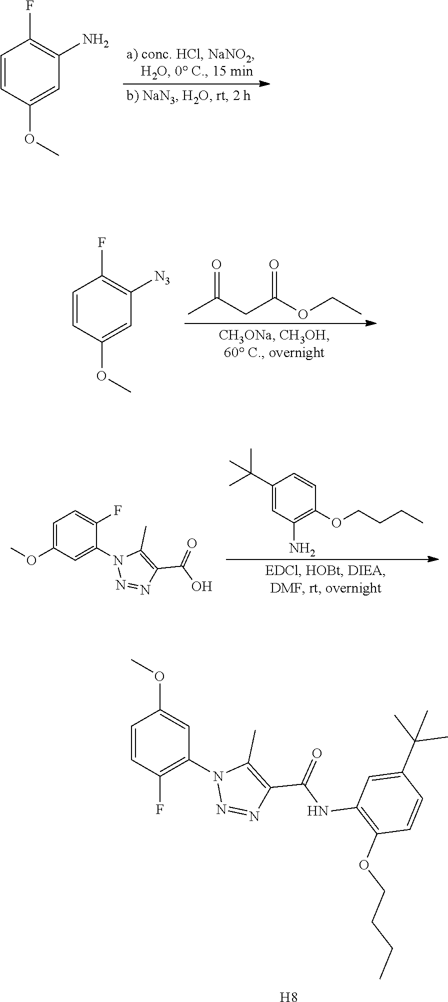

The preparation of compound H8 was prepared by 2-fluoro-5-methoxyaniline as start compound according to the procedure for A1 synthesis. White solid, 349.6 mg, 77% yield (final step). ¹H NMR (400 MHz, Chloroform-d) δ 9.79 (s, 1H), 8.63 (d, J=2.4 Hz, 1H), 7.24 (t, J=9.2 Hz, 1H), 7.13-7.05 (m, 2H), 7.01 (dd, J=5.7, 3.1 Hz, 1H), 6.86 (d, J=8.6 Hz, 1H), 4.09 (t, J=6.5 Hz, 2H), 3.85 (s, 3H), 2.63 (d, J=1.7 Hz, 3H), 1.88 (dq, J=8.4, 6.5 Hz, 2H), 1.64-1.57 (m, 2H), 1.36 (s, 9H), 1.01 (t, J=7.4 Hz, 3H). ¹³C NMR (101 MHz, CDCl3) δ 158.99, 156.23, 156.21, 151.57, 149.12, 145.72, 143.82, 138.85, 138.75, 127.40, 123.56, 123.42, 120.25, 118.01, 117.93, 117.57, 117.36, 117.07, 112.87, 110.60, 68.52, 56.11, 34.45, 31.58, 31.30, 19.24, 13.86, 9.14. ESI-TOF HRMS: m/z 455.2456 (C₂₅H₃₁FN₄O₃+H⁺ requires 455.2453).

rrrrrr. Preparation of H9

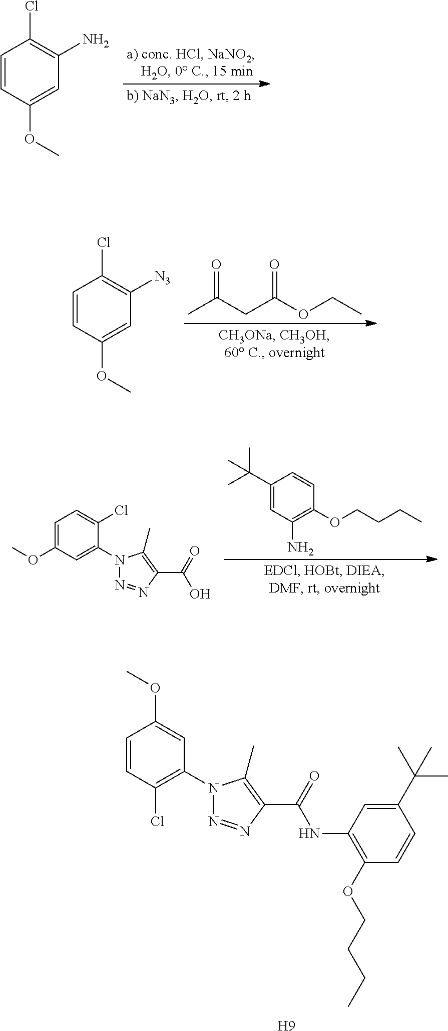

The preparation of compound H9 was prepared by 2-chloro-5-methoxyaniline as start compound according to the procedure for A1 synthesis. White solid, 372.1 mg, 79% yield (final step). ¹H NMR (400 MHz, Chloroform-d) δ 9.82 (s, 1H), 8.66 (d, J=2.4 Hz, 1H), 7.48 (d, J=9.0 Hz, 1H), 7.13-7.05 (m, 2H), 6.97 (d, J=3.0 Hz, 1H), 6.86 (d, J=8.6 Hz, 1H), 4.09 (t, J=6.4 Hz, 2H), 3.83 (s, 3H), 2.58 (s, 3H), 1.88 (dq, J=8.4, 6.5 Hz, 2H), 1.60 (h, J=7.4 Hz, 2H), 1.36 (s, 9H), 1.01 (t, J=7.4 Hz, 3H). ¹³C NMR (101 MHz, CDCl3) δ 159.07, 159.02, 145.71, 143.80, 138.74, 138.69, 133.64, 131.06, 127.43, 122.72, 120.25, 118.13, 117.03, 114.38, 110.62, 68.54, 55.97, 34.45, 31.60, 31.32, 19.26, 13.89, 9.28. ESI-TOF HRMS: m/z 471.2163 (C₂₅H₃₁ClN₄O₃+H⁺ requires 471.2158).

ssssss. Preparation of H10

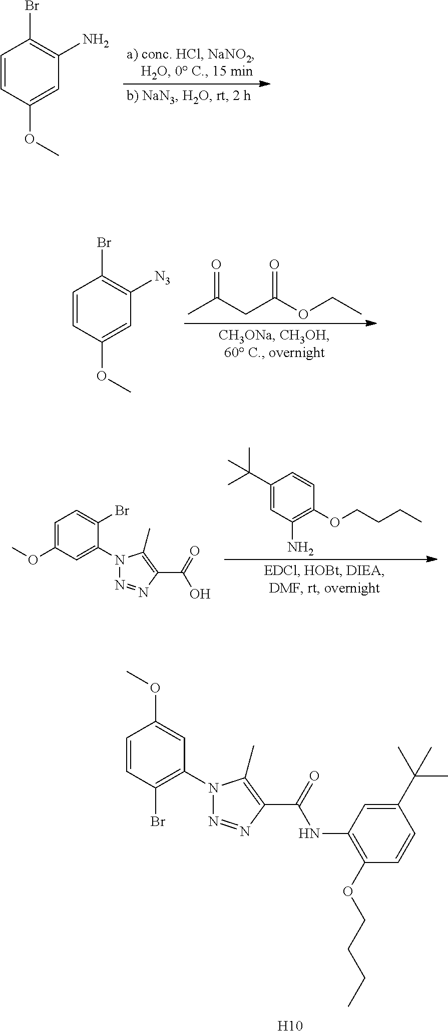

The preparation of compound H10 was prepared by 2-bromo-5-methoxyaniline as start compound according to the procedure for A1 synthesis. White solid, 402.8 mg, 78% yield (final step). ¹H NMR (400 MHz, Chloroform-d) δ 9.83 (s, 1H), 8.66 (d, J=2.4 Hz, 1H), 7.63 (d, J=8.9 Hz, 1H), 7.07 (dd, J=8.5, 2.4 Hz, 1H), 7.02 (dd, J=8.9, 2.9 Hz, 1H), 6.96 (d, J=2.9 Hz, 1H), 6.86 (d, J=8.5 Hz, 1H), 4.08 (t, J=6.4 Hz, 2H), 3.82 (s, 3H), 2.57 (s, 3H), 1.88 (dq, J=8.5, 6.5 Hz, 2H), 1.66-1.54 (m, 2H), 1.36 (s, 9H), 1.01 (t, J=7.4 Hz, 3H). ¹³C NMR (101 MHz, CDCl3) δ 159.68, 159.08, 145.70, 143.79, 138.70, 138.55, 135.36, 134.11, 127.44, 120.24, 118.41, 117.01, 114.73, 111.47, 110.63, 68.54, 55.94, 34.45, 31.60, 31.32, 19.27, 13.90, 9.41. ESI-TOF HRMS: m/z 515.1655 (C₂₅H₃₁BrN₄O₃+H⁺ requires 515.1653).

tttttt. Preparation of H11

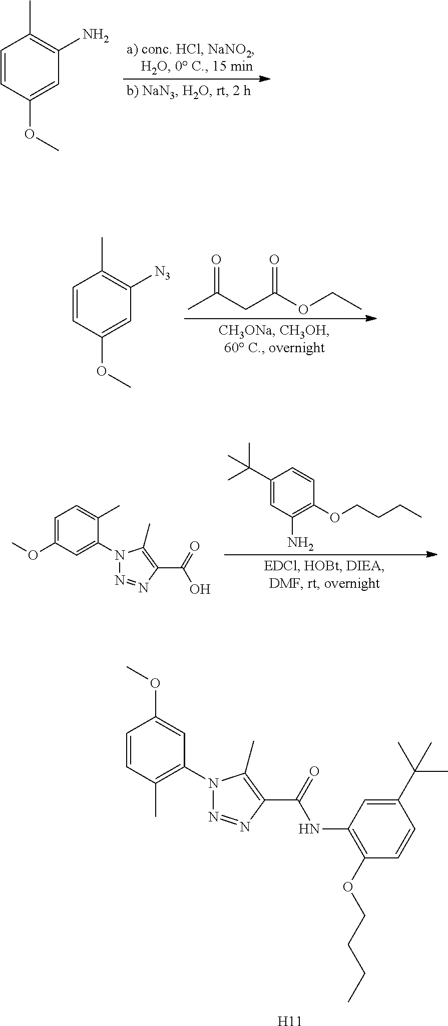

The preparation of compound H11 was prepared by 5-methoxy-2-methylaniline as start compound according to the procedure for A1 synthesis. White solid, 153.2 mg, 68% yield. ¹H NMR (400 MHz, Chloroform-d) δ 9.82 (s, 1H), 8.65 (d, J=2.4 Hz, 1H), 7.30 (d, J=8.6 Hz, 1H), 7.06 (ddd, J=16.1, 8.5, 2.5 Hz, 2H), 6.86 (d, J=8.5 Hz, 1H), 6.79 (d, J=2.7 Hz, 1H), 4.09 (t, J=6.5 Hz, 2H), 3.82 (s, 3H), 2.52 (s, 3H), 1.98 (s, 3H), 1.93-1.83 (m, 2H), 1.67-1.54 (m, 2H), 1.36 (s, 9H), 1.01 (t, J=7.4 Hz, 3H). ¹³C NMR (101 MHz, CDCl3) δ 159.27, 158.41, 145.73, 143.80, 138.71, 137.85, 134.91, 132.03, 127.46, 127.11, 120.20, 117.05, 116.83, 112.49, 110.60, 68.54, 55.66, 34.45, 31.59, 31.31, 19.26, 16.29, 13.88, 9.25. ESI-TOF HRMS: m/z 510.2720 (C₂₆H₃₄N₄O₃+H⁺ requires 510.2711).

uuuuuu. Preparation of H12

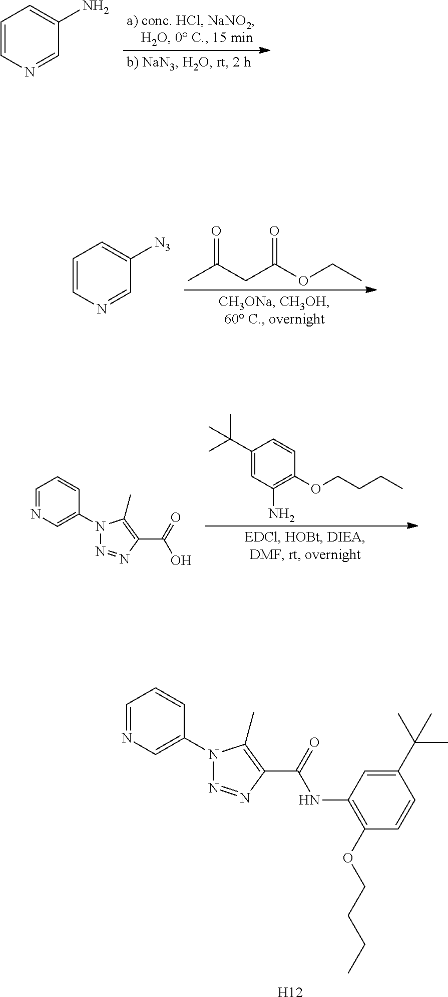

The preparation of compound H12 was prepared by pyridin-3-amine as start compound according to the procedure for A1 synthesis. White solid, 360.7 mg, 88% yield. ¹H NMR (400 MHz, Chloroform-d) δ 9.78 (s, 1H), 8.80 (dd, J=6.8, 3.7 Hz, 2H), 8.62 (d, J=2.4 Hz, 1H), 7.86 (ddd, J=8.2, 2.6, 1.5 Hz, 1H), 7.54 (dd, J=8.2, 4.8 Hz, 1H), 7.06 (dd, J=8.5, 2.4 Hz, 1H), 6.85 (d, J=8.5 Hz, 1H), 4.08 (d, J=6.4 Hz, 2H), 2.73 (s, 3H), 1.86 (dq, J=8.2, 6.5 Hz, 2H), 1.57 (h, J=7.4 Hz, 2H), 1.34 (s, 9H), 0.99 (t, J=7.4 Hz, 3H). ¹³C NMR (101 MHz, CDCl3) δ 158.77, 151.03, 145.87, 145.69, 143.81, 139.56, 137.20, 132.57, 132.50, 127.26, 124.18, 120.41, 117.02, 110.66, 68.52, 34.44, 31.58, 31.28, 19.23, 13.86, 9.80. ESI-TOF HRMS: m/z 408.2386 (C₂₃H₂₉N₅O₂+H⁺ requires 408.2394).

vvvvvv. Preparation of H13

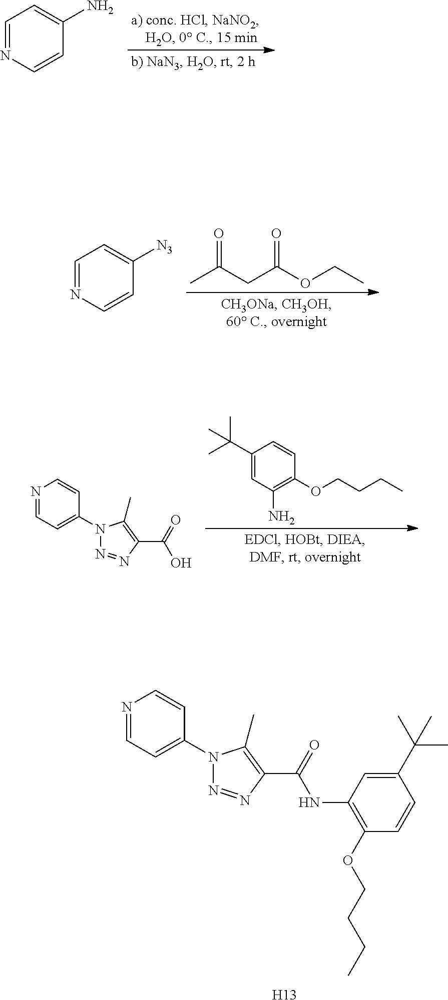

The preparation of compound H13 was prepared by pyridin-4-amine as start compound according to the procedure for A1 synthesis. White solid, 382.8 mg, 94% yield. ¹H NMR (400 MHz, Chloroform-d) δ 9.78 (s, 1H), 8.86 (d, J=5.6 Hz, 2H), 8.61 (d, J=2.4 Hz, 1H), 7.59-7.48 (m, 2H), 7.08 (dd, J=8.5, 2.4 Hz, 1H), 6.85 (d, J=8.5 Hz, 1H), 4.08 (t, J=6.4 Hz, 2H), 2.82 (s, 3H), 1.97-1.75 (m, 2H), 1.58 (h, J=7.5 Hz, 2H), 1.35 (s, 9H), 1.00 (t, J=7.4 Hz, 3H). ¹³C NMR (101 MHz, CDCl3) δ 158.64, 151.63, 145.70, 143.84, 142.68, 139.93, 136.77, 127.20, 120.48, 118.51, 117.04, 110.65, 68.52, 34.44, 31.57, 31.27, 19.23, 13.86, 10.18. ESI-TOF HRMS: m/z 408.2396 (C₂₃H₂₉N₅O₂+H⁺ requires 408.2394).

wwwwww. Preparation of H14

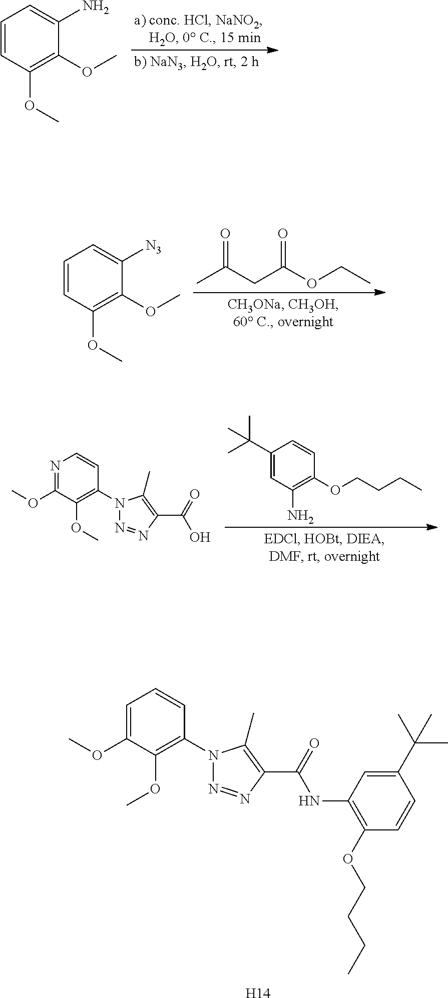

The preparation of compound H14 was prepared by 2,3-dimethoxyaniline as start compound according to the procedure for A1 synthesis. White solid, 401.8 mg, 86% yield. ¹H NMR (400 MHz, Chloroform-d) δ 9.83 (s, 1H), 8.67 (d, J=2.4 Hz, 1H), 7.20 (t, J=8.1 Hz, 1H), 7.11 (dd, J=8.4, 1.5 Hz, 1H), 7.06 (dd, J=8.5, 2.4 Hz, 1H), 6.98 (dd, J=7.9, 1.6 Hz, 1H), 6.85 (d, J=8.6 Hz, 1H), 4.08 (t, J=6.5 Hz, 2H), 3.92 (s, 3H), 3.64 (s, 3H), 2.55 (s, 3H), 1.88 (dq, J=8.5, 6.6 Hz, 2H), 1.65-1.53 (m, 2H), 1.35 (s, 9H), 1.00 (t, J=7.4 Hz, 3H). ¹³C NMR (101 MHz, CDCl3) δ 159.37, 153.49, 145.68, 144.38, 143.78, 139.07, 138.47, 129.50, 127.53, 124.36, 120.12, 119.65, 117.02, 114.46, 110.61, 68.53, 61.49, 56.19, 34.44, 31.59, 31.31, 19.25, 13.88, 9.33. ESI-TOF HRMS: m/z 467.2657 (C₂₆H₃₄N₄O₄+H⁺ requires 467.2653).

xxxxxx. Preparation of H15

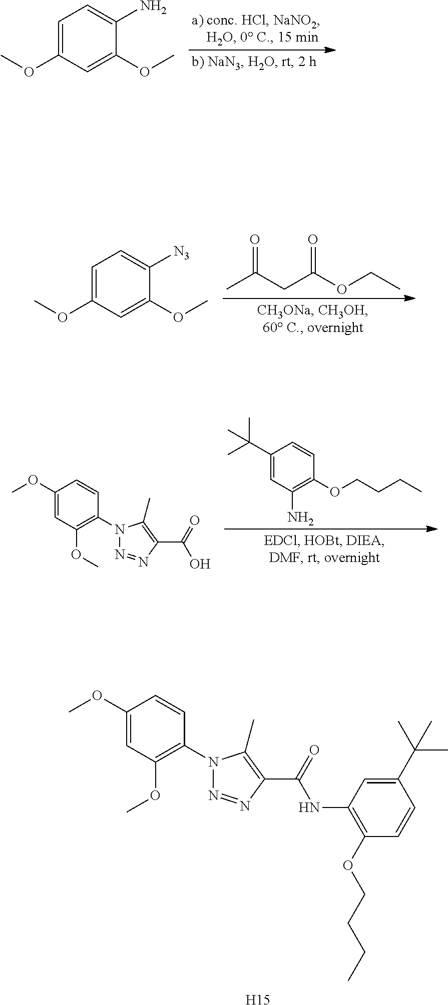

The preparation of compound H15 was prepared by 2,4-dimethoxyaniline as start compound according to the procedure for A1 synthesis. White solid, 379.5 mg, 81% yield. ¹H NMR (400 MHz, Chloroform-d) δ 9.82 (s, 1H), 8.65 (t, J=1.9 Hz, 1H), 7.33-7.21 (m, 1H), 7.06 (dd, J=8.6, 2.4 Hz, 1H). 6.85 (d, J=8.5 Hz, 1H). 6.67-6.54 (m, 2H), 4.08 (t, J=6.5 Hz, 2H), 3.88 (s, 3H), 3.78 (s, 3H), 2.50 (d, J=1.0 Hz, 3H), 1.88 (p, J=6.7 Hz, 2H), 1.59 (h, J=7.4 Hz, 2H), 1.36 (s, 9H), 1.00 (t, J=7.4 Hz, 3H). ¹³C NMR (101 MHz, CDCl3) δ 162.52, 159.54, 155.17, 145.72, 143.75, 139.15, 138.37, 129.12, 127.59, 120.03, 117.48, 117.03, 110.56, 104.81, 99.46, 68.50, 55.83, 55.74, 34.44, 31.60, 31.32, 19.25, 13.88, 9.24.

ESI-TOF HRMS: m/z 467.2656 (C₂₆H₃₄N₄O₄+H⁺ requires 467.2653).

yyyyyy. Preparation of H16

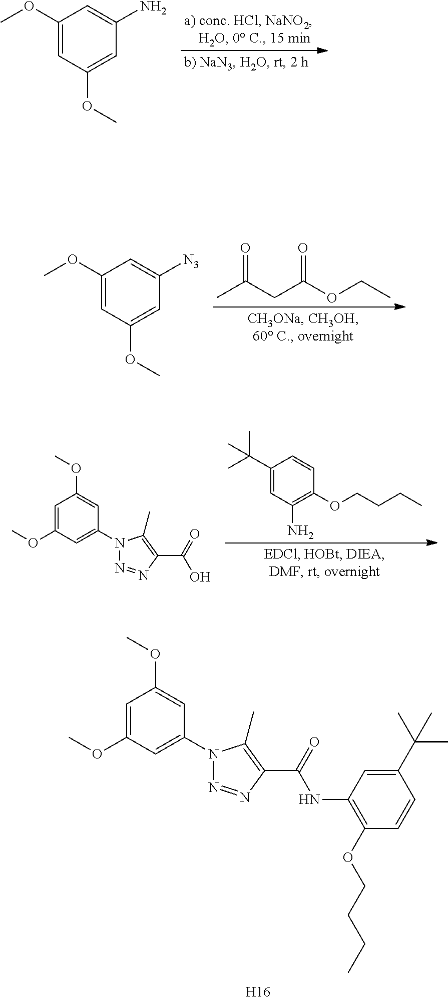

The preparation of compound H16 was prepared by 3,5-dimethoxyaniline as start compound according to the procedure for A1 synthesis. White solid, 363.4 mg, 78% yield. ¹H NMR (400 MHz, Chloroform-d) δ 9.81 (s, 1H), 8.65 (d, J=2.4 Hz, 1H), 7.05 (dd, J=8.5, 2.4 Hz, TH), 6.84 (d, J=8.5 Hz, TH), 6.60 (dd, J=7.7, 2.2 Hz, 3H), 4.07 (t, J=6.4 Hz, 2H), 3.81 (s, 6H), 2.71 (s, 3H), 1.87 (dq, J=8.2, 6.5 Hz, 2H), 1.58 (h, J=7.4 Hz, 2H), 1.35 (s, 9H), 1.00 (t, J=7.4 Hz, 3H). ¹³C NMR (101 MHz, CDCl3) δ 161.33, 159.11, 145.68, 143.75, 139.16, 137.05, 136.93, 127.45, 120.21, 116.96, 110.63, 103.68, 101.67, 68.49, 55.69, 34.43, 31.58, 31.31, 19.25, 13.88, 9.95. ESI-TOF HRMS: m/z 467.2657 (C₂₆H₃₄N₄O₄+H⁺ requires 467.2653).

zzzzzz. Preparation of H17

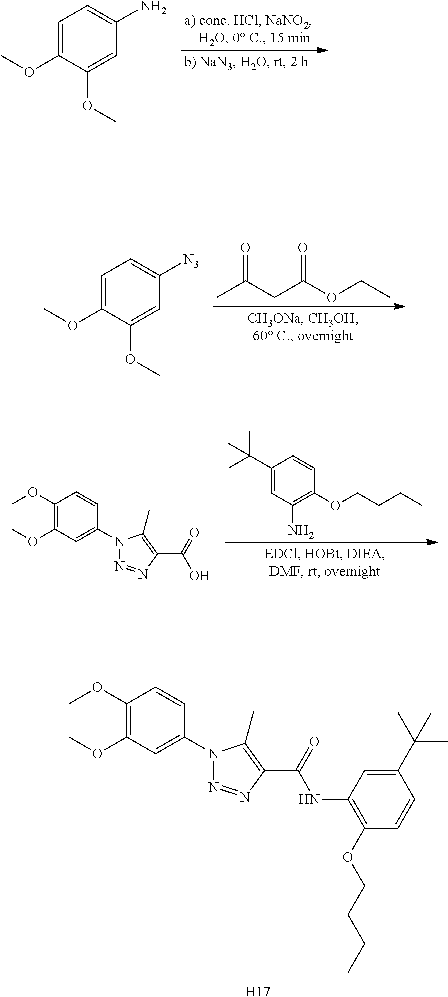

The preparation of compound H17 was prepared by 3,4-dimethoxyaniline as start compound according to the procedure for A1 synthesis. White solid, 382.7 mg, 82% yield. ¹H NMR (400 MHz, Chloroform-d) δ 9.80 (s, 1H), 8.63 (d, J=2.4 Hz, 1H), 7.06 (dd, J=8.5, 2.4 Hz, 1H), 7.02-6.97 (m, 3H), 6.85 (d, 0.1=8.5 Hz, 1H), 4.07 (t, J=6.5 Hz, 2H), 3.95 (s, 3H), 3.92 (s, 3H), 2.67 (s, 3H), 1.87 (dq, J=8.4, 6.5 Hz, 2H), 1.65-1.52 (m, 2H), 1.35 (s, 9H), 1.00 (t, J=7.4 Hz, 3H). ¹³C NMR (101 MHz, CDCl3) δ 159.21, 150.27, 149.68, 145.70, 143.79, 139.05, 137.09, 128.53, 127.45, 120.20, 117.62, 116.99, 111.01, 110.62, 109.03, 68.50, 56.24, 56.21, 34.44, 31.58, 31.29, 19.23, 13.87, 9.87. ESI-TOF HRMS: m/z 467.2656 (C₂₆H₃₄N₄O₄+H⁺ requires 467.2653).

aaaaaaa. Preparation of H18

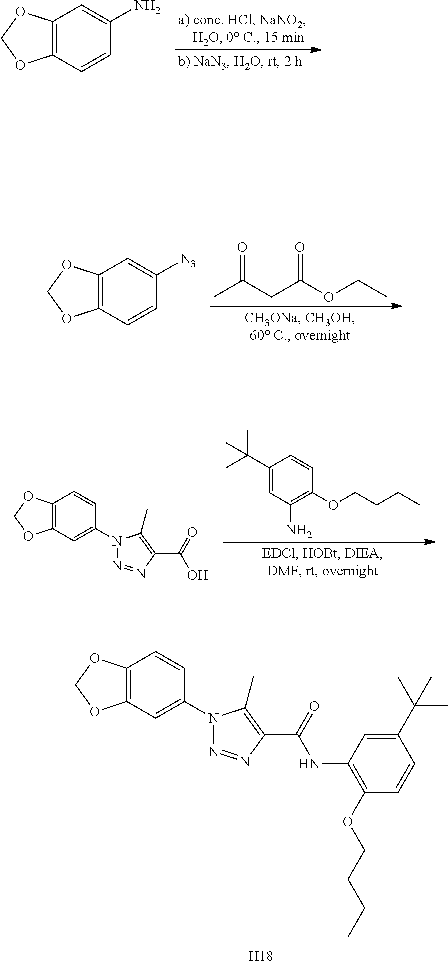

The preparation of compound H18 was prepared by benzo[d][1.3]dioxol-5-amine as start compound according to the procedure for A1 synthesis. White solid, 374.9 mg, 83% yield. ¹H NMR (400 MHz, Chloroform-d) δ 9.80 (s, 1H), 8.64 (d, J=2.7 Hz, 1H), 7.13-7.01 (m, 1H), 6.91 (s, 3H), 6.85 (d, J=8.5 Hz, 1H), 6.06 (s, 2H), 4.07 (t, J=6.5 Hz, 2H), 2.66 (s, 3H), 1.88 (h, J=8.1, 6.9 Hz, 2H), 1.59 (p, J=7.3 Hz, 2H), 1.35 (s, 9H), 1.00 (t, J=7.3 Hz, 3H). ¹³C NMR (101 MHz, CDCl3) δ 159.15, 148.96, 148.50, 145.69, 143.76, 139.00, 137.09, 129.33, 127.44, 120.22, 119.27, 116.99, 110.63, 108.46, 106.58, 102.33, 68.50, 34.44, 31.59, 31.30, 19.25, 13.88, 9.82. ESI-TOF HRMS: m/z 451.2339 (C₂₅H₃₀N₄O₄+H⁺ requires 451.2340).

bbbbbbb. Preparation of H19

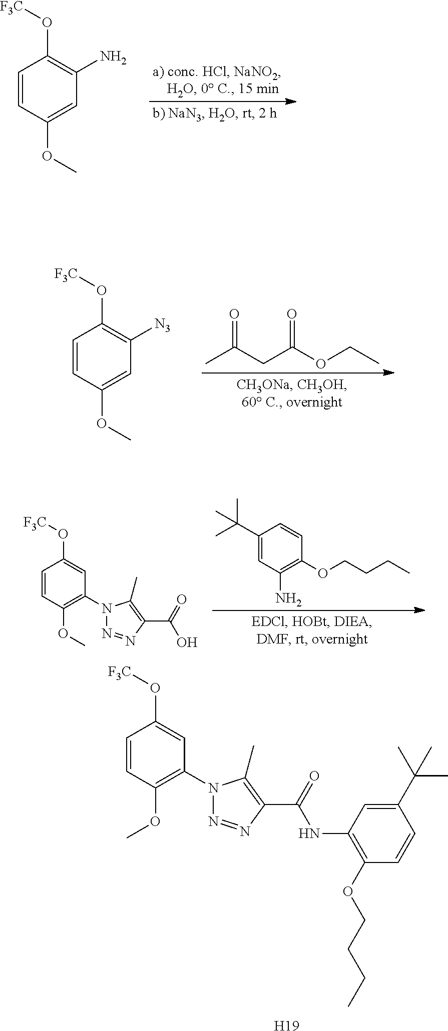

The preparation of compound H19 was prepared by 5-methoxy-2-(trifluoromethoxy)aniline as start compound according to the procedure for A1 synthesis. White solid, 422.9 mg, 81% yield. ¹H NMR (400 MHz, Chloroform-d) δ 9.82 (s, 1H), 8.66 (d, J=2.4 Hz, 1H), 7.42 (ddt, J=9.2, 3.0, 1.0 Hz, 1H), 7.35 (dd, J=3.0, 1.0 Hz, 1H), 7.14-7.06 (m, 2H), 6.86 (d, J=8.6 Hz, 1H), 4.08 (t, J=6.5 Hz, 2H), 3.82 (s, 3H), 2.55 (s, 3H), 1.94-1.82 (m, 2H), 1.68-1.55 (m, 2H), 1.36 (s, 9H), 1.01 (t, J=7.4 Hz, 3H). ¹³C NMR (101 MHz, CDCl3) δ 159.16, 152.69, 145.71, 143.79, 142.20, 142.18, 142.15, 142.13, 139.10, 138.65, 127.45, 124.80, 124.64, 121.96, 121.74, 120.23, 119.18, 117.01, 112.91, 110.63, 68.51, 56.31, 34.43, 31.56, 31.31, 19.24, 13.84, 9.24. ESI-TOF HRMS: m/z 521.2375 (C₂₆H₃₁F₃N₄O₄+H⁺ requires 521.2370).

ccccccc. Preparation of H20

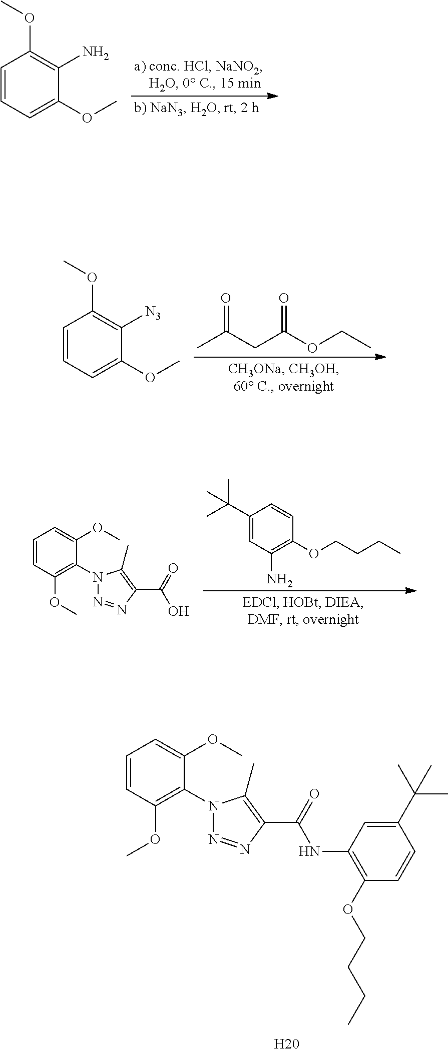

The preparation of compound 1120 was prepared by 2,6-dimethoxyaniline as start compound according to the procedure for A1 synthesis. White solid, 375.2 mg, 80% yield. ¹H NMR (400 MHz, Chloroform-d) δ 9.85 (s, 1H), 8.67 (d, J=2.4 Hz, 1H), 7.45 (t, J=8.5 Hz, 1H), 7.06 (dd, J=8.5, 2.4 Hz, 1H), 6.85 (d, J=8.6 Hz, 1H), 6.71 (s, 1H), 6.69 (s, 1H), 4.08 (t, J=6.5 Hz, 2H), 3.77 (s, 6H), 2.45 (s, 3H), 1.88 (dq, J=8.5, 6.5 Hz, 2H), 1.66-1.54 (m, 2H), 1.36 (d, J=0.6 Hz, 9H), 1.00 (t, J=7.4 Hz, 3H). ¹³C NMR (101 MHz, CDCl3) δ 159.65, 156.28, 145.74, 143.72, 139.50, 138.17, 132.09, 127.69, 119.91, 117.01, 112.86, 110.50, 104.28, 68.49, 56.16, 34.44, 31.60, 31.34, 19.26, 13.89, 8.87. ESI-TOF HRMS: m/z 467.2639 (C₂₆H₃₄N₄O₄+H⁺ requires 467.2653).

ddddddd. Preparation of I1

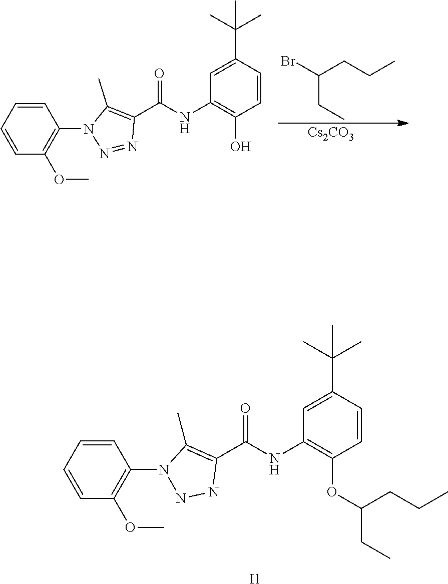

The preparation of compound I1 was prepared by reaction of N-(5-(tert-butyl)-2-hydroxyphenyl)-1-(2-methoxyphenyl)-5-methyl-1H-1,2,3-triazole-4-carboxamide and 3-bromohexane according to the procedure for B4 synthesis. 108.1 mg, 89% yield, white solid. ¹H NMR (500 MHz, CDCl3) δ 9.89 (s, 1H), 8.66 (d, J=2.5 Hz, 1H), 7.54 (ddd, J=8.4, 7.6, 1.7 Hz, 1H), 7.39 (dd, J=7.8, 1.7 Hz, 1H), 7.13 (td, J=7.6, 1.2 Hz, 1H), 7.10 (dd. J=8.4, 1.1 Hz, 1H), 7.05 (dd, J=8.6, 2.5 Hz, 1H), 6.84 (d, J=8.6 Hz, 1H), 4.27 (p, J=5.8 Hz, 1H), 3.82 (s, 3H), 2.53 (s, 3H), 1.83-1.63 (m, 4H), 1.54-1.43 (m, 2H), 1.36 (s, 9H), 1.01 (t, J=7.4 Hz, 3H), 0.94 (t, J=7.4 Hz, 3H). ¹³C NMR (126 MHz, CDCl3) δ 159.47, 154.02, 145.05, 143.59, 138.98, 138.54, 131.97, 128.52, 128.38, 124.32, 121.04, 120.04, 117.02, 112.14, 112.04, 80.29, 55.84, 35.52, 34.45, 31.61, 26.58, 18.70, 14.23, 9.59, 9.29.

eeeeeee. Preparation of I2

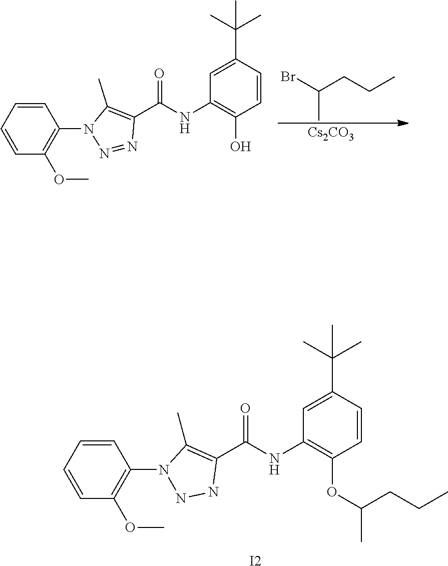

The preparation of compound I2 was prepared by reaction of N-(5-(tert-butyl)-2-hydroxyphenyl)-1-(2-methoxyphenyl)-5-methyl-1H-1,2,3-triazole-4-carboxamide and 2-bromopentane according to the procedure for B4 synthesis. 89.8 mg, 75% yield, white solid. ¹H NMR (500 MHz, CDCl3) δ 9.85 (s, 1H), 8.66 (d, J=2.4 Hz, 1H), 7.54 (ddd, J=8.3, 7.5, 1.7 Hz, 1H), 7.39 (dd, J=7.8, 1.7 Hz, 1H), 7.13 (td, J=7.7, 1.2 Hz, 1H), 7.10 (dd, J=8.6, 1.1 Hz, 1H), 7.06 (dd, J=8.5, 2.4 Hz, 1H), 6.86 (d, J=8.6 Hz, 1H), 4.43 (h, J=6.1 Hz, 1H), 3.81 (s, 3H), 2.53 (s, 3H), 1.96-1.78 (m, 1H), 1.69-1.60 (m, 1H), 1.57-1.45 (m, 2H), 1.37 (d, J=6.1 Hz, 3H), 1.36 (s, 9H), 0.96 (t, J=7.4 Hz, 3H). ¹³C NMR (126 MHz, CDCl3) δ 159.46, 154.03, 144.72, 143.79, 139.00, 138.53, 131.99, 128.51, 128.49, 124.31, 121.05, 120.10, 117.08, 112.38, 112.16, 75.37, 55.84, 38.68, 34.46, 31.60, 19.92, 18.80, 14.14, 9.29.

fffffff. Preparation of 13

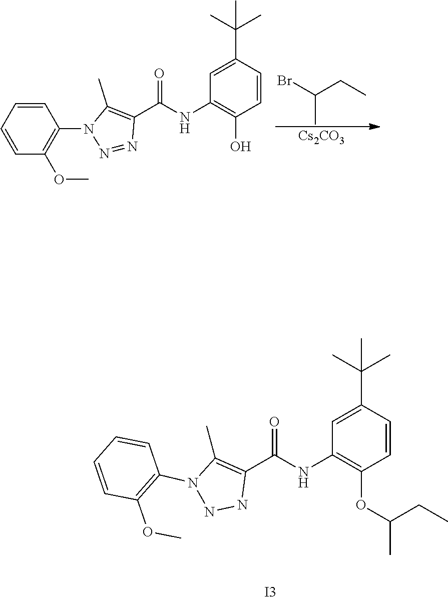

The preparation of compound 13 was prepared by reaction of N-(5-(tert-butyl)-2-hydroxyphenyl)-1-(2-methoxyphenyl)-5-methyl-1H-1,2,3-triazole-4-carboxamide and 2-bromobutane according to the procedure for B4 synthesis. 95.8 mg, 83% yield, white solid. ¹H NMR (500 MHz, CDCl3) δ 9.87 (s, 1H), 8.66 (d, J=2.4 Hz, 1H), 7.54 (ddd, J=8.4, 7.6, 1.7 Hz, 1H), 7.14 (dd, J=7.6, 1.2 Hz, 1H), 7.10 (dd, J=8.4, 1.1 Hz, 1H), 7.06 (dd, J=8.5, 2.4 Hz, 1H), 6.86 (d, J=8.6 Hz, 1H), 4.37 (h, J=6.0 Hz, 1H), 3.81 (s, 3H), 2.53 (s, 3H), 1.93-1.81 (m, 1H), 1.79-1.69 (m, 1H), 1.38 (d, J=6.1 Hz, 3H), 1.36 (s, 9H), 1.05 (t, J=7.4 Hz, 3H). ¹³C NMR (126 MHz, CDCl3) δ 158.41, 152.98, 143.66, 142.79, 137.96, 137.49, 130.95, 127.47, 123.27, 120.00, 119.05, 116.03, 111.39, 111.11, 75.68, 54.80, 33.42, 30.56, 28.27, 18.36, 8.74, 8.25.

ggggggg. Preparation of J1

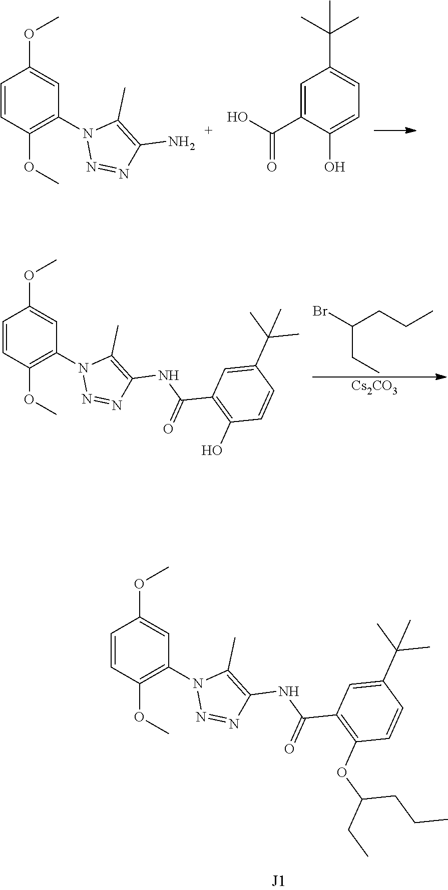

To a solution of 5-(tert-butyl)-2-hydroxybenzoic acid (500 mg, 2.57 mmol) in DCM (10 mL) was added oxalochloride (279 mg, 2.57 mmol) at 0° C. The reaction mixture was warmed to room temperature and stirred for 3 hours. The reaction mixture was concentrated under the reduced pressure. The residue was added to a solution of 1-(2,5-dimethoxyphenyl)-5-methyl-1H-1,2,3-triazol-4-amine (603 mg, 2.57 mmol) and N-ethyl-N-isopropylpropan-2-amine (998 mg, 7.72 mmol) in DCM 10 mL at 0° C. The reaction mixture was stirred at reflux for overnight. Then the reaction mixture was added EA 50 mL and washed by water, brin, dried by MgSO₄ and concentrated under the reduced pressure. The residue was purified by column chromatography (0% to 100% acetonitrile in water) to get a white solid (317.6 mg. 30% yield). ¹H NMR (500 MHz, CDCl3) δ 10.23 (s, 1H), 10.03 (s, 1H), 7.91 (dd, J=17.9, 2.4 Hz, 1H), 7.55-7.44 (m, 1H), 7.09 (dd, J=9.0, 2.8 Hz, 1H), 7.03 (d, J=9.2 Hz, 1H), 7.00-6.96 (m, 2H), 3.82 (s, 3H), 3.79 (d, 0.1=1.4 Hz, 3H), 2.25 (s, 3H), 1.31 (s, 9H). ¹³C NMR (126 MHz, CDCl3) δ 168.64, 159.26, 153.67, 148.06, 142.12, 142.10, 138.53, 132.46, 130.53, 124.82, 123.90, 117.95, 117.79, 113.66, 113.34, 56.38, 56.03, 34.35, 31.47, 9.67.

To a solution of 5-(tert-butyl)-N-(1-(2,5-dimethoxyphenyl)-5-methyl-1H-1,2,3-triazol-4-yl)-2-hydroxybenzamide (100 mg, 0.244 mmol) in acetone 5 mL was added Cs₂CO₃ (159 mg, 0.487 mmol) and 3-bromohexane (48.3 mg, 0.292 mmol). The suspension was stirred at 60° C. for overnight. The reaction mixture was then diluted with water (50 mL) and extracted with EtOAc (50 mL×2). The EtOAc layer was washed with water, dried with anhydrous Na₂SO₄, and concentrated. The residue was purified by silica gel chromatography (0% to 100% EA in hexane) to give product as a white solid (102.3 mg, 85% yield). ¹H NMR (500 MHz, CDCl3) δ 10.23 (s, 1H), 8.30 (d, J=2.7 Hz, 1H), 7.50 (dd, J=8.7, 2.8 Hz, 1H), 7.06 (dd, J=9.1, 2.9 Hz, 1H), 7.03-6.95 (m, 3H), 4.49 (p, J=5.9 Hz, 1H), 3.81 (s, 3H), 3.78 (s, 3H), 2.27 (s, 3H), 1.86-1.73 (m, 4H), 1.60-1.43 (m, 2H), 1.34 (s, 9H), 1.05 (t, J=7.4 Hz, 3H), 0.97 (t, J=7.4 Hz, 3H). ¹³C NMR (126 MHz, CDCl3) δ 163.92, 154.76, 153.66, 148.19, 143.86, 139.66, 130.51, 129.39, 128.96, 125.55, 120.60, 117.34, 113.75, 113.69, 113.46, 81.43, 56.51, 56.00, 35.53, 34.29, 31.38, 26.65, 18.79, 14.12, 9.80, 9.72.

hhhhhhh. Preparation of J2

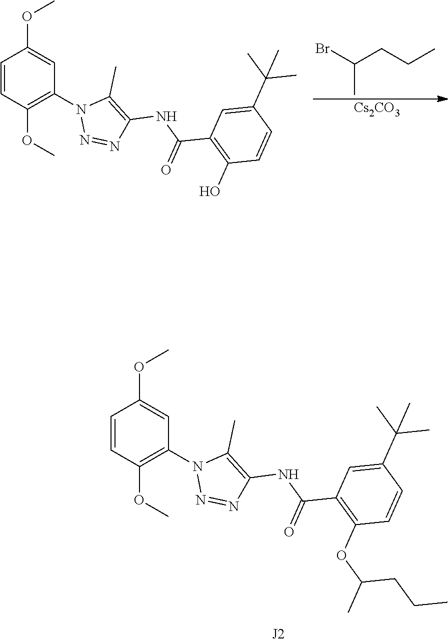

The preparation of compound J2 was prepared by reaction of 5-(tert-butyl)-N-(1-(2,5-dimethoxyphenyl)-5-methyl-1H-1,2,3-triazol-4-yl)-2-hydroxybenzamide and 2-bromopentaneaccording to the procedure for J1 synthesis. 83.5 mg, 71% yield, white solid. ¹H NMR (500 MHz, CDCl3) δ 10.20 (s, 1H), 8.30 (d, J=2.7 Hz, 1H), 7.50 (dd, J=8.7, 2.7 Hz, 1H), 7.05 (dd, J=9.1, 2.9 Hz, 1H), 7.01 (d, J=9.1 Hz, 1H), 7.00-6.95 (m, 2H), 4.65 (h, J=6.1 Hz, 1H), 3.80 (s, 3H), 3.78 (s, 3H), 2.27 (s, 3H), 1.99-1.91 (m, 1H), 1.74 (ddt, J=13.7, 9.7, 6.0 Hz, 1H), 1.63-1.48 (m, 2H), 1.47 (d, J=6.1 Hz, 3H), 1.34 (s, 9H), 0.98 (t, J=7.4 Hz, 3H). ¹³C NMR (126 MHz, CDCl3) δ 163.90, 154.33, 153.65, 148.20, 144.00, 139.75, 130.49, 129.36, 128.91, 125.58, 120.76, 117.28, 113.85, 113.79, 113.45, 76.38, 56.49, 55.99, 38.52, 34.30, 31.38, 19.93, 18.91, 14.01, 9.70.

iiiiii. Preparation of J3

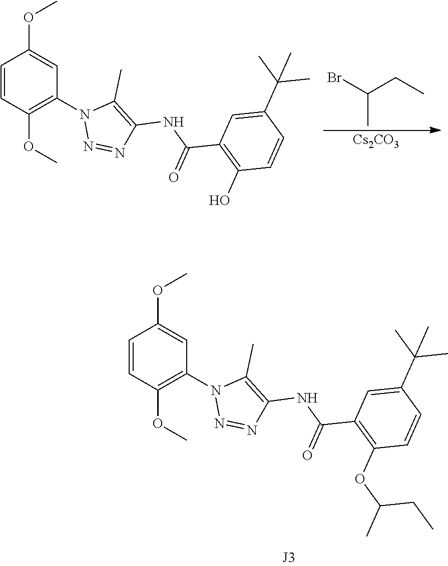

The preparation of compound J3 was prepared by reaction of 5-(tert-butyl)-N-(1-(2,5-dimethoxyphenyl)-5-methyl-1H-1,2,3-triazol-4-yl)-2-hydroxybenzamide and 2-bromobutane to the procedure for J1 synthesis. 71.8 mg, 63% yield, white solid. ¹H NMR (500 MHz, CDCl3) δ 10.21 (s, 1H), 8.31 (d, J=2.6 Hz, 1H), 7.50 (dd, J=8.7, 2.7 Hz, 1H), 7.05 (dd, J=9.1, 2.8 Hz, 1H), 7.01 (d, J=9.1 Hz, 1H), 6.99-6.96 (m, 2H), 4.58 (h, J=6.1 Hz, 1H), 3.80 (s, 3H), 3.78 (s, 3H), 2.27 (s, 3H), 2.01-1.91 (m, 1H), 1.87-1.74 (m, 1H), 1.47 (d, J=6.1 Hz, 3H), 1.34 (s, 9H), 1.07 (t, J=7.4 Hz, 3H). ¹³C NMR (126 MHz, CDCl3) δ 163.89, 154.32, 153.65, 148.21, 144.01, 139.78, 130.47, 129.37, 128.90, 125.59, 120.75, 117.27, 113.88, 113.81, 113.45, 77.79, 56.50, 56.00, 34.30, 31.38, 29.30, 19.44, 9.98, 9.71.

jjjjjjj. Preparation of K1

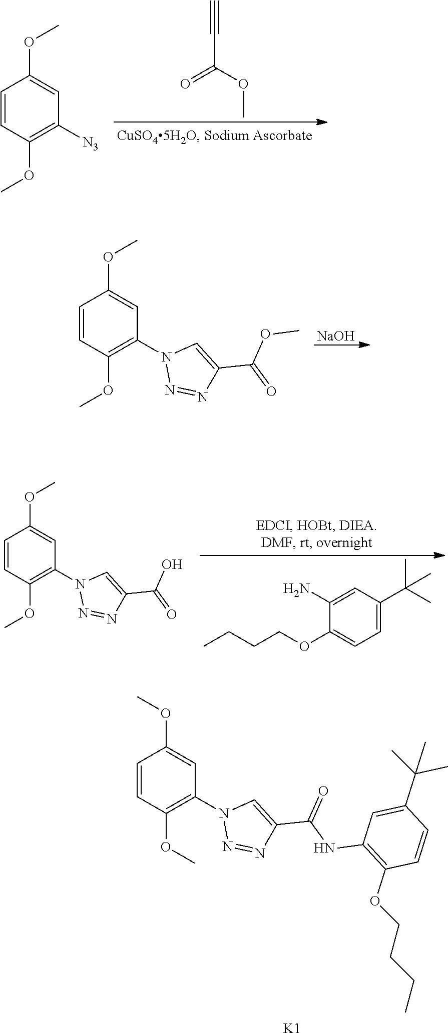

2-azido-1,4-dimethoxybenzene (2.014 g, 11.24 mmol), copper sulfate pentahydrate (0.140 g, 0.562 mmol), sodium ascorbate (0.445 g, 2.248 mmol) and methyl propiolate (1 ml, 11.24 mmol) were dissolved in CH₃OH (20 ml). The reaction was stirred under N₂ protect for 12 h. Then the reaction mixture filtered and concentrated. The residue was purified by reverse silica gel chromatography (0% to 100% acetonitrile in water) to give product (2.16 g, 73% yield). ¹H NMR (400 MHz, DMSO-d6) δ 8.22 (d, J=1.2 Hz, 1H), 6.47-6.42 (m, 2H), 6.31 (ddd, J=9.1, 3.1, 1.1 Hz, 1H), 3.04 (s, 3H), 2.96 (s, 3H), 2.94 (s, 3H), 2.50 (s, 6H). ¹³C NMR (101 MHz, DMSO) δ 160.59, 153.04, 145.78, 138.46, 130.79, 125.15, 116.50, 114.19, 111.42, 56.56, 55.84, 51.91.

To a solution of Methyl 1-(2,5-dimethoxyphenyl)-1H-1,2,3-triazole-4-carboxylate (2.16 g, 8.27 mmol) was dissolved in CH₃OH (30 mL) and water (30 mL) was added sodium hydroxide (3.31 g, 83 mmol. The reaction was stirred at room temperature for 12 hours. Then the reaction mixture was adjusted to pH 4 with 5N HCl. The reaction mixture was stirred at room temperature for 0.5 hour and filtered. The solid was dried to get a brown solid, 1.53 g, 75% yield. ¹H NMR (400 MHz, DMSO-d6) δ 8.95 (s, 1H), 7.33-7.20 (m, 2H), 7.14 (dd, J=9.2, 3.0 Hz, 1H), 3.80 (s, 3H), 3.78 (s, 3H). ¹³C NMR (101 MHz, DMSO) δ 161.55, 153.04, 145.76, 139.59, 130.58, 125.29, 116.37, 114.20, 111.40, 56.56, 55.84.

To a solution of 1-(2,5-dimethoxyphenyl)-1H-1,2,3-triazole-4-carboxylic acid (0.249 g, 1 mmol) in DMF 5 mL was added 2-butoxy-5-(tert-butyl)aniline (0.221 g, 1.000 mmol), HOBt, 80% (0.203 g, 1.200 mmol) and EDCI (0.288 g, 1.500 mmol), followed by N-ethyl-N-isopropylpropan-2-amine (0.331 ml, 2.000 mmol). The suspension was stirred for overnight. The reaction mixture was then diluted with water (20 mL) and extracted with EtOAc (25 mL×2). The EtOAc layers were washed with water, dried with anhydrous Na₂SO₄ and concentrated. The residue was purified by silica gel chromatography (0% to 100% EA in hexane) to give product as a white solid, 389.5 mg, 86% yield. ¹H NMR (400 MHz, Chloroform-d) δ 9.71 (s, 1H), 8.78 (d, J=1.6 Hz, 1H), 8.69 (d, J=2.4 Hz, 1H), 7.48 (d, J=2.9 Hz, 1H), 7.08 (dt, J=8.5, 2.0 Hz, 1H), 7.03 (d, J=9.1 Hz, 1H), 6.97 (dd, J=9.1, 2.9 Hz, 1H), 6.86 (d, J=8.3 Hz, 1H), 4.09 (t, J=6.4 Hz, 2H), 3.85 (d, J=1.6 Hz, 3H), 3.82 (d, J=1.6 Hz, 3H), 1.89 (ddt, J=10.0, 8.1, 4.1 Hz, 2H), 1.65-1.54 (m, 2H), 1.35 (d, J=1.7 Hz, 9H), 1.01 (t, J=7.4 Hz, 3H). ¹³C NMR (101 MHz, CDCl3) δ 157.99, 153.88, 145.70, 144.77, 143.81, 143.56, 127.65, 127.20, 125.89, 120.48, 117.31, 116.10, 113.62, 110.63, 110.09, 68.54, 56.43, 55.97, 34.44, 31.52, 31.29, 19.25, 13.87. ESI-TOF HRMS: m/z 453.2500 (C₂₅H₃₂N₄O₄+H⁺ requires 453.2497).

kkkkkk. Preparation of K2

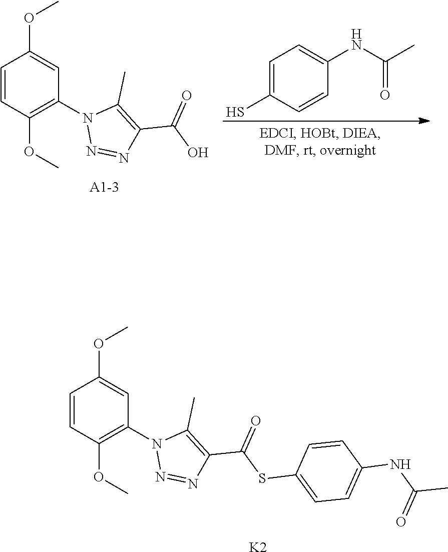 [SMILES: COc1ccc(OC)c(-n2nnc(C(=O)O)c2C)c1 ; CC(=O)Nc1ccc(S)cc1 ; COc1ccc(OC)c(-n2nnc(C(=O)Sc3ccc(NC(C)=O)cc3)c2C)c1 ; "K2"]

The preparation of compound K2 was prepared by reaction of 1-(2,5-dimethoxyphenyl)-5-methyl-1H-1,2,3-triazole-4-carboxylic acid and N-(4-mercaptophenyl)acetamide according to the procedure for A1 synthesis. White solid, 73 mg, 9% yield. ¹H NMR (400 MHz, Chloroform-d) δ 7.61 (t, J=7.1 Hz, 3H), 7.50-7.45 (m, 2H), 7.08 (dd, J=9.1, 3.0 Hz, 1H), 7.02 (d, J=9.1 Hz, 1H), 6.94 (d, J=3.0 Hz, 1H), 3.80 (s, 3H), 3.75 (s, 3H), 2.39 (s, 3H), 2.18 (s, 3H). ¹³C NMR (101 MHz, CDCl₃) δ 185.30, 168.45, 153.73, 147.87, 141.25, 139.39, 138.64, 136.09, 123.99, 121.67, 120.21, 117.64, 113.74, 113.40, 56.38, 56.01, 24.69, 9.30. ESI-TOF HRMS: m/z 413.1267 (C₂₀H₂₀N₄O₄S+H⁺ requires 413.1278).

llllll. Preparation of K3

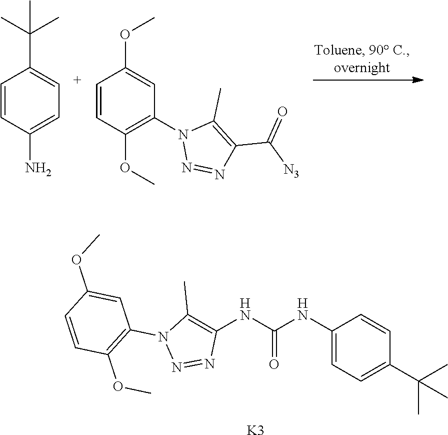

A suspension of 1-(2,5-dimethoxyphenyl)-5-methyl-1H-1,2,3-triazole-4-carbonyl azide (200 mg, 0.694 mmol) and 4-(tert-butyl)aniline (207 mg, 1.388 mmol) in toluene (3 mL) was stirred at 90° C. for overnight. Then the solvent was removed, and the residue was diluted with NaHCO₃ saturated solution (100 mL). The mixture was extracted with CH₂Cl₂ (3×50 mL). The combined extracts were washed with brine (50 mL), dried by MgSO₄ and concentrated. The residue was purified by column chromatography (0% to 100% EA in hexane) to give product as a light brown solid, 145.3 mg, 51% yield. ¹H NMR (500 MHz, DMSO) δ 8.83 (s, 1H), 8.41 (s, 1H), 7.39-7.35 (m, 2H), 7.31-7.27 (m, 2H), 7.26 (d, J=9.2 Hz, 1H), 7.18 (dd, J=9.1, 3.1 Hz, 1H), 7.02 (d, J=3.1 Hz, 1H), 3.77 (s, 3H), 3.75 (s, 3H), 2.05 (s, 3H), 1.26 (s, 9H). ¹³C NMR (126 MHz, DMSO) δ 153.53, 153.44, 148.08, 144.75, 140.42, 137.48, 128.67, 125.83, 125.36, 118.72, 117.39, 114.33, 114.25, 56.72, 56.29, 34.36, 31.73, 8.67.

mmmmmmm. Preparation of K4

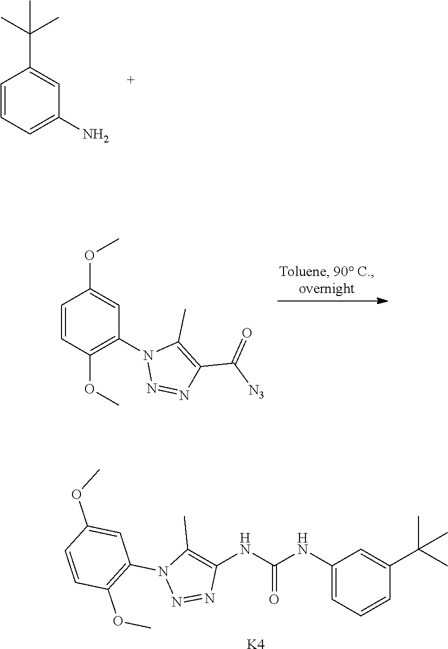 [SMILES: CC(C)(C)c1cccc(N)c1 ; COc1ccc(OC)c(-n2nnc(C(=O)N=[N+]=[N-])c2C)c1 ; COc1ccc(OC)c(-n2nnc(NC(=O)Nc3cccc(C(C)(C)C)c3)c2C)c1 ; "K4"]

The preparation of compound K4 was prepared by reaction of 1-(2,5-dimethoxyphenyl)-5-methyl-1H-1,2,3-triazole-4-carbonyl azide and 3-(tert-butyl)aniline according to the procedure for K3 synthesis. 122.4 mg, white solid, 43%. ¹H NMR (500 MHz, DMSO) δ 9.33 (s, 1H), 8.84 (s, 1H), 7.90 (t, J=2.0 Hz, 1H), 7.77 (ddd, J=8.0, 2.0, 0.9 Hz, 1H), 7.71 (d, J=9.2 Hz, 1H), 7.67-7.61 (m, 2H), 7.49-7.43 (m, 2H), 4.23 (s, 3H), 4.21 (s, 3H), 2.50 (s, 3H), 1.72 (s, 9H). ¹³C NMR (126 MHz, DMSO) δ 153.53, 153.50, 151.72, 148.09, 140.33, 139.84, 128.90, 128.86, 125.37, 119.45, 117.40, 116.18, 115.86, 114.34, 114.25, 56.72, 56.29, 34.86, 31.60, 8.65.

nnnnnnn. Preparation of K5

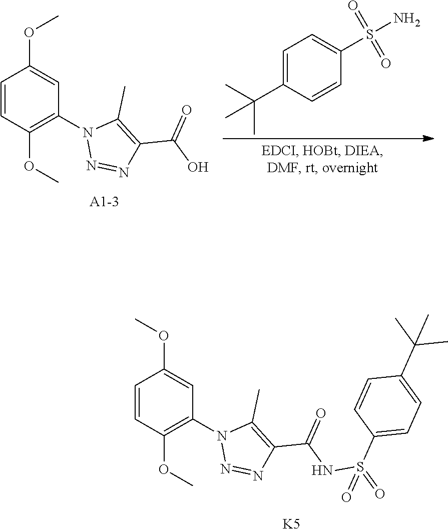

To a solution of 1-(2,5-dimethoxyphenyl)-5-methyl-1H-1,2,3-triazole-4-carboxylic acid (0.263 g, 1 mmol) in DMF 5 mL was added 4-(tert-butyl)benzenesulfonamide (0.213 g, 1.000 mmol), HOBt, 80% (0.203 g, 1.200 mmol) and EDCI (0.288 g, 1.500 mmol), followed by N-ethyl-N-isopropylpropan-2-amine (0.331 ml, 2.000 mmol) at room temperature. The suspension was stirred for overnight. The solvent was diluted with EtOAc (100 mL), washed by NaHCO₃ solution, water, 1N HCl and brine. The organic phase dried by MgSO₄, filtered and concentrated. The residue was purified to providing N-((4-(tert-butyl)phenyl)sulfonyl)-1-(2,5-dimethoxyphenyl)-5-methyl-1H-1,2,3-triazole-4-carboxamide (371.5 mg, 81% yield) as a white solid. ¹H NMR (400 MHz, DMSO-d6) δ 12.51 (s, 1H), 7.98-7.94 (m, 2H), 7.70-7.65 (m, 2H), 7.26 (d, J=9.2 Hz, 1H), 7.20 (dd, J=9.2, 3.0 Hz, 1H), 7.09 (d, J=3.0 Hz, 1H), 3.75 (s, 3H), 3.71 (s, 3H), 2.24 (s, 3H), 1.31 (s, 9H). ¹³C NMR (101 MHz, DMSO) δ 159.54, 156.71, 153.00, 147.56, 140.95, 136.83, 135.68, 127.54, 125.97, 123.23, 117.52, 113.90, 113.85, 56.26, 55.83, 34.97, 30.72, 8.84. ESI-TOF HRMS: m/z 459.1697 (C₂₂H₂₆N₄O₅S+H⁺ requires 459.1697).

oooooo. Preparation of K6

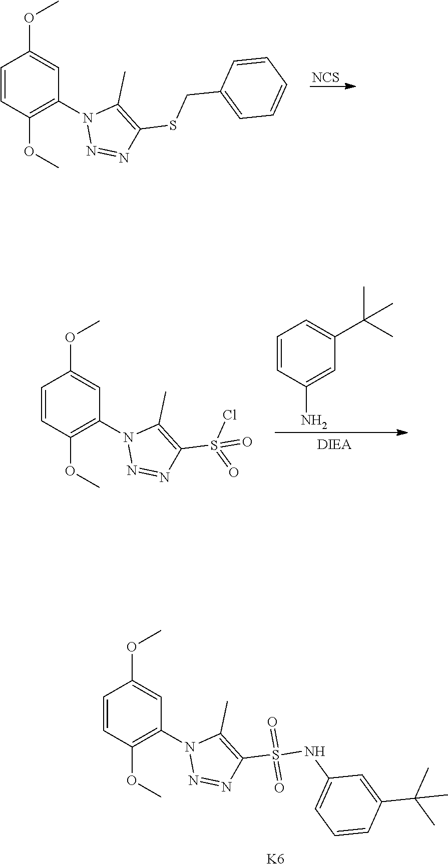

To a solution of 4-(benzylthio)-1-(2,5-dimethoxyphenyl)-5-methyl-1H-1,2,3-triazole (84.4 mg, 0.247 mmol) in acetic acid (2 mL) and H₂O (1 mL) was added N-chlorosuccinamide (132 mg, 0.989 mmol) was added at ice bath. The resulting reaction mixture was warmed to room temperature and stirred for 1 hour. Upon completion, the reaction mixture was diluted with water and extracted with ethyl acetate (2×10 mL). The combined organic extracts were washed with water, brine, dried by Na₂SO₄ and concentrated in vacuo. The crude product was without purified for next step.

To a solution of the residue in DMF (2 mL) was added 3-(tert-butyl)aniline (0.074 g, 0.494 mmol) and N-ethyl-N-isopropylpropan-2-amine (0.064 g, 0.494 mmol) at 0° C. The resulting reaction mixture was warmed to room temperature and stirred for 8 hours. Upon completion, the reaction mixture was diluted with water and extracted with ethyl acetate. The combined organic extracts were washed with water, brine, dried by MgSO₄ and concentrated in vacuo. The crude product was purified by column chromatography to give N-(3-(tert-butyl)phenyl)-1-(2,5-dimethoxyphenvl)-5-methyl-1H-1,2,3-triazole-4-sulfonamide 56.3 mg, yield 53%, as a white solid. ¹H NMR (500 MHz, DMSO) δ 10.56 (s, 1H), 7.24 (d, J=9.2 Hz, 1H), 7.22-7.17 (m, 3H), 7.13 (dt, J=8.0, 1.6 Hz, 1H), 7.06 (d, J=3.0 Hz, 1H), 6.94 (ddd, J=7.9, 2.2, 1.1 Hz, 1H), 3.74 (s, 3H), 3.65 (s, 3H), 2.12 (s, 3H), 1.21 (s, 9H). ¹³C NMR (126 MHz, DMSO) δ 153.54, 152.14, 147.97, 142.43, 138.66, 137.34, 129.20, 123.66, 121.78, 118.48, 118.31, 118.18, 114.49, 114.17, 56.80, 56.33, 34.84, 31.41, 8.85. ESI-TOF HRMS: m/z 431.1748 (C₂₁H₂₆N₄O₄S+H⁺ requires 431.1748).

ppppppp. Preparation of K8

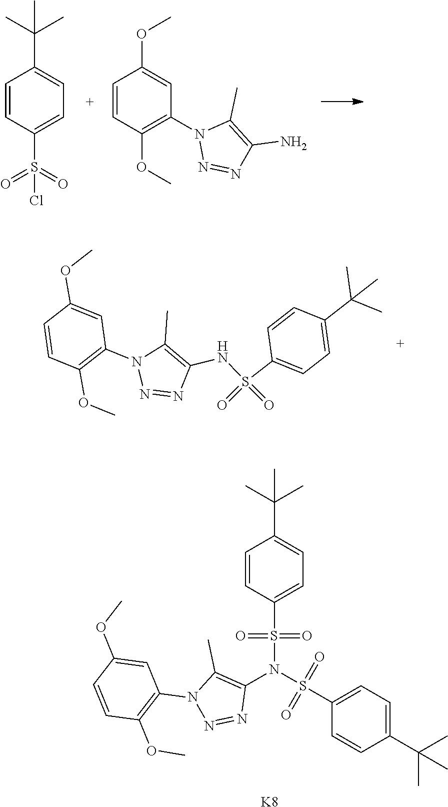 [SMILES: CC(C)(C)c1ccc(S(=O)(=O)Cl)cc1 ; COc1ccc(OC)c(-n2nnc(N)c2C)c1 ; COc1ccc(OC)c(-n2nnc(NS(=O)(=O)c3ccc(C(C)(C)C)cc3)c2C)c1 ; COc1ccc(OC)c(-n2nnc(N(S(=O)(=O)c3ccc(C(C)(C)C)cc3)S(=O)(=O)c3ccc(C(C)(C)C)cc3)c2C)c1 ; "K8"]

To a solution of 1-(2,5-dimethoxyphenyl)-5-methyl-1H-1,2,3-triazol-4-amine (100 mg, 0.427 mmol) in DMF 5 mL at rt was added 4-(tert-butyl)benzenesulfonyl chloride (99 mg, 0.427 mmol) and N-ethyl-N-isopropylpropan-2-amine (110 mg, 0.854 mmol). The suspension was stirred at room temperature for overnight. The reaction mixture was then poured into water (50 mL) and extracted with EtOAc (50 mL×2). The EtOAc layers were washed with water, dried with anhydrous Na₂SO₄ and concentrated. The residue was purified by silica gel chromatography (0% to 100% EA in hexane) to give product.

4-(tert-butyl)-N-((4-(tert-butyl)phenyl)sulfonyl)-N-(1-(2,5-dimethoxyphenyl)-5-methyl-1H-1,2,3-triazol-4-yl)benzenesulfonamide white solid, 51.8 mg, 39%. ¹H NMR (500 MHz, CDCl3) δ 7.89-7.82 (m, 4H), 7.54-7.49 (m, 4H), 7.06 (dd, J=9.0, 3.0 Hz, 1H), 7.02 (d, J=3.0 Hz, 1H), 7.00 (d, J=9.1 Hz, 1H), 3.82 (s, 3H), 3.78 (s, 3H), 1.82 (s, 3H), 1.35 (s, 18H). ¹³C NMR (126 MHz, CDCl3) δ 158.12, 153.82, 147.68, 137.27, 136.02, 135.91, 128.68, 126.00, 125.18, 117.67, 113.56, 113.49, 56.57, 56.05, 35.34, 31.09, 7.72. [00697]4-(tert-butyl)-N-(1-(2,5-dimethoxyphenyl)-5-methyl-1H-1,2,3-triazol-4-yl)benzenesulfonamide, white solid, 78.9 mg, 43%. ¹H NMR (500 MHz, CDCl3) δ 7.73-7.68 (m, 2H), 7.53-7.48 (m, 2H), 7.06 (dd, J=9.1, 3.0 Hz, 1H), 7.01 (d, J=9.1 Hz, 1H), 6.94 (d, J=3.0 Hz, 1H), 6.52 (s, 1H), 3.81 (s, 3H), 3.78 (s, 3H), 2.26 (s, 3H), 1.33 (s, 9H). 13C NMR (126 MHz, CDCl3) δ 156.96, 153.69, 147.90, 137.51, 136.15, 133.63, 127.35, 126.11, 125.19, 117.37, 113.67, 113.33, 56.42, 56.03, 35.20, 31.08, 8.32.

qqqqqqq. Preparation of L1

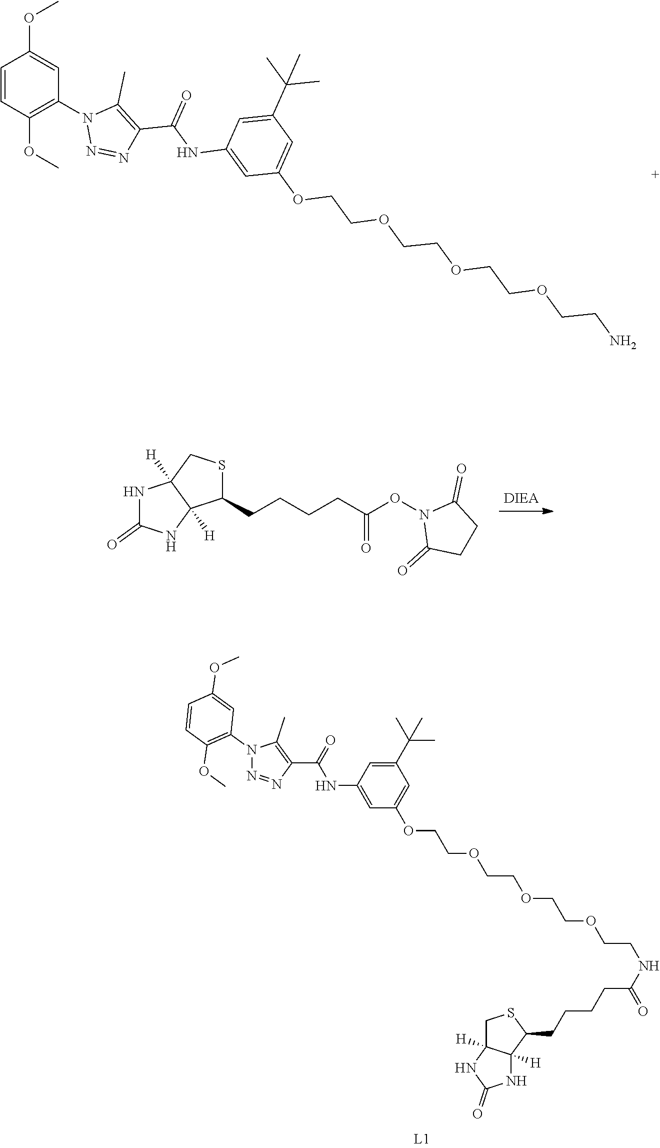 [SMILES: COc1ccc(OC)c(-n2nnc(C(=O)Nc3cc(OCCOCCOCCOCCN)cc(C(C)(C)C)c3)c2C)c1 ; O=C1N[C@H]2[C@H](CS[C@H]2CCCCC(=O)ON2C(=O)CCC2=O)N1 ; COc1ccc(OC)c(-n2nnc(C(=O)Nc3cc(OCCOCCOCCOCCNC(=O)CCCC[C@@H]4SC[C@@H]5NC(=O)N[C@@H]54)cc(C(C)(C)C)c3)c2C)c1]

To a solution of N-(3-(2-(2-(2-(2-aminoethoxy)ethoxy)ethoxy)ethoxy)-5-(tert-butyl)phenyl)-1-(2,5-dimethoxyphenyl)-5-methyl-1H-1,2,3-triazole-4-carboxamide (100 mg, 0.171 mmol) in DMF 5 mL was added 2,5-dioxopyrrolidin-1-yl 5-((3aS,4S,6aR)-2-oxohexahydro-1H-thieno[3,4-d]imidazol-4-yl)pentanoate (69.9 mg, 0.205 mmol) and followed by N-ethyl-N-isopropylpropan-2-amine (44.1 mg, 0.341 mmol). The suspension was stirred at room temperature for overnight. The reaction mixture was purified by reverse column to afford a white solid, 110.0 mg, yield 79%. ¹H NMR (400 MHz, Chloroform-d) δ 9.12 (s, 1H), 7.37 (t, J=2.1 Hz, 1H), 7.18 (s, 1H), 7.10-6.97 (m, 2H), 6.93 (d, J=3.0 Hz, 1H), 6.74 (t, J=2.0 Hz, 1H), 6.62 (t, J=5.4 Hz, 1H), 6.38 (s, 1H), 5.68 (s, 1H), 4.50-4.38 (m, 1H), 4.29-4.19 (m, 1H), 4.15 (t, J=4.9 Hz, 2H), 3.84 (t, J=4.8 Hz, 2H), 3.79 (s, 2H), 3.73 (s, 3H), 3.72-3.69 (m, 2H), 3.69-3.56 (m, 6H), 3.54 (t, J=5.1 Hz, 2H), 3.39 (q, J=5.3 Hz, 2H), 3.08 (td, J=7.3, 4.5 Hz, 1H), 2.84 (dd, J=12.7, 5.0 Hz, 1H), 2.69 (d, J=12.8 Hz, 1H), 2.49 (s, 3H), 2.17 (t, J=7.5 Hz, 2H), 1.77-1.53 (m, 4H), 1.40 (q, J=7.5 Hz, 2H), 1.30 (s, 9H). ¹³C NMR (101 MHz, CDCl3) δ 171.59, 162.45, 157.74, 157.49, 152.19, 151.86, 146.47, 137.72, 136.97, 136.31, 122.87, 115.85, 112.34, 111.90, 108.18, 107.39, 101.37, 69.15, 68.90, 68.82, 68.52, 68.25, 68.18, 65.95, 60.20, 58.61, 54.77, 54.37, 53.83, 38.76, 37.55, 34.25, 33.22, 29.59, 26.53, 26.46, 23.84, 7.49. ESI-TOF HRMS: m/z 812.4008 (C₄₀H₅₇N₇O₉S+H⁺ requires 812.4016).

rrrrrrr. Preparation of L2

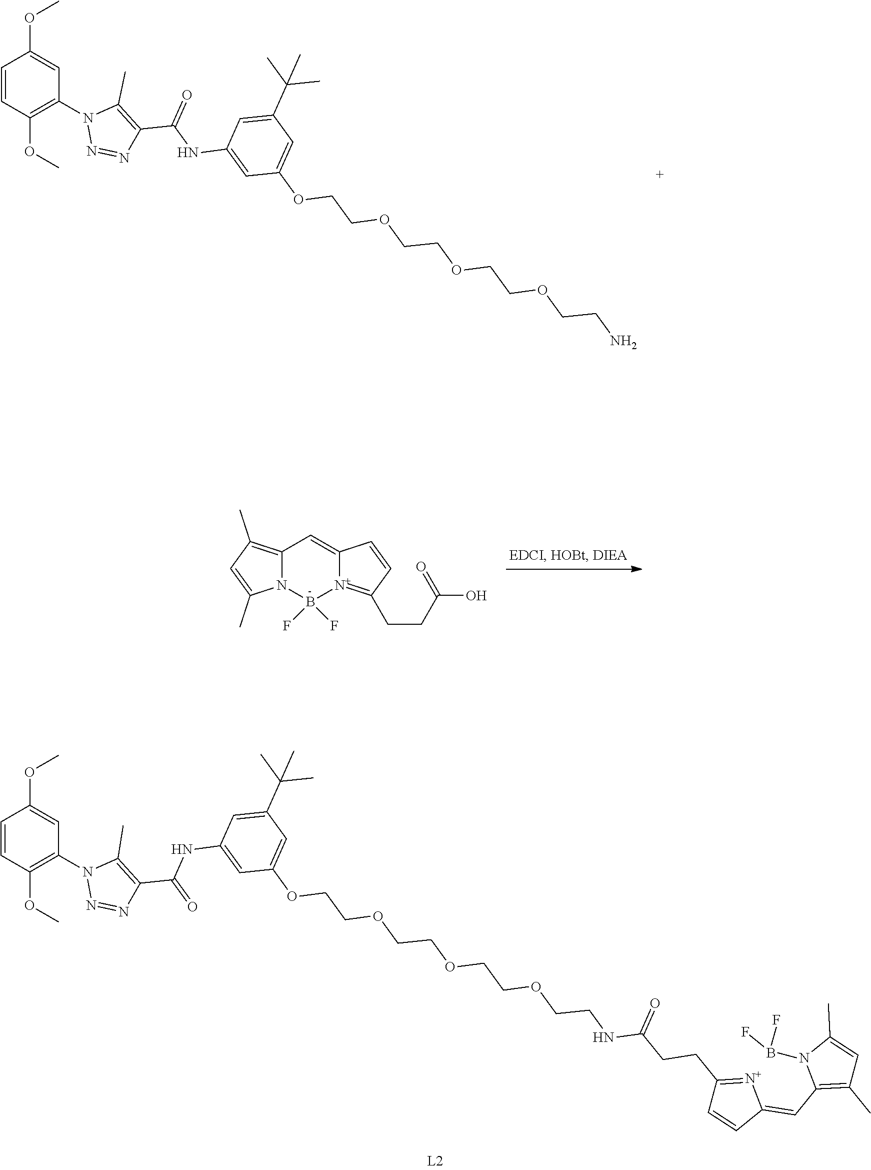

To a solution of N-(3-(2-(2-(2-(2-aminoethoxy)ethoxy)ethoxy)ethoxy)-5-(tert-butyl)phenyl)-1-(2,5-dimethoxyphenyl)-5-methyl-1H-1,2,3-triazole-4-carboxamide (100 mg, 0.171 mmol) in DMF 5 mL at rt was added 3-(5,5-difluoro-7,9-dimethyl-5H-414,514-dipyrrolo[1,2-c:2′,1′-1][1,3,2]diazaborinin-3-yl)propanoic acid (49.9 mg, 0.171 mmol), HOBt, 80% (34.6 mg, 0.205 mmol) and EDCI (49.1 mg, 0.256 mmol), followed by N-ethyl-N-isopropylpropan-2-amine (44.1 mg, 0.341 mmol). The suspension was stirred at room temperature for overnight. The reaction mixture was diluted with water (25 mL) and extracted with EtOAc (50 mL). The EtOAc layer was washed with water, dried with anhydrous Na₂SO₄ and concentrated. The residue was purified by column chromatography (0% to 100% acetonitrile in water) to give product (66.5 mg, 45% yield). ¹H NMR (400 MHz, Chloroform-d) δ 9.05 (s, 1H), 7.37 (s, 1H), 7.16 (s, 1H), 7.12-6.99 (m, 3H), 6.94 (s, 1H), 6.85 (s, 1H), 6.75 (s, 1H), 6.33-6.22 (m, 1H), 6.12 (m, 2H), 4.22-4.08 (m, 2H), 3.89-3.78 (m, 5H), 3.75 (s, 3H), 3.71 (s, 2H), 3.67-3.54 (m, 6H), 3.51 (d, J=5.2 Hz, 2H), 3.42 (t, J=5.3 Hz, 2H), 3.27 (t, J=7.6 Hz, 2H), 2.61 (t, J=7.6 Hz, 2H), 2.54 (s, 2H), 2.51 (s, 3H), 2.22 (s, 3H), 2.15 (s, 3H), 1.31 (s, 9H). ¹³C NMR (101 MHz, CDCl3) δ 171.65, 159.95, 159.33, 159.20, 157.98, 153.86, 153.49, 148.15, 143.53, 139.31, 138.57, 137.98, 135.03, 133.46, 128.25, 124.58, 123.68, 120.22, 117.50, 117.45, 113.99, 113.56, 109.65, 109.10, 102.92, 70.84, 70.60, 70.54, 70.31, 69.80, 69.79, 67.58, 56.42, 56.01, 39.32, 35.81, 34.87, 31.23, 30.73, 24.79, 14.80, 11.14, 9.13. ESI-TOF HRMS: m/z 860.4332 (C₄₄H₅₆BF₂N₇O₈+H^{|} requires 860.4329).

sssssss. Preparation of B34, M1, M2, M3, and M4

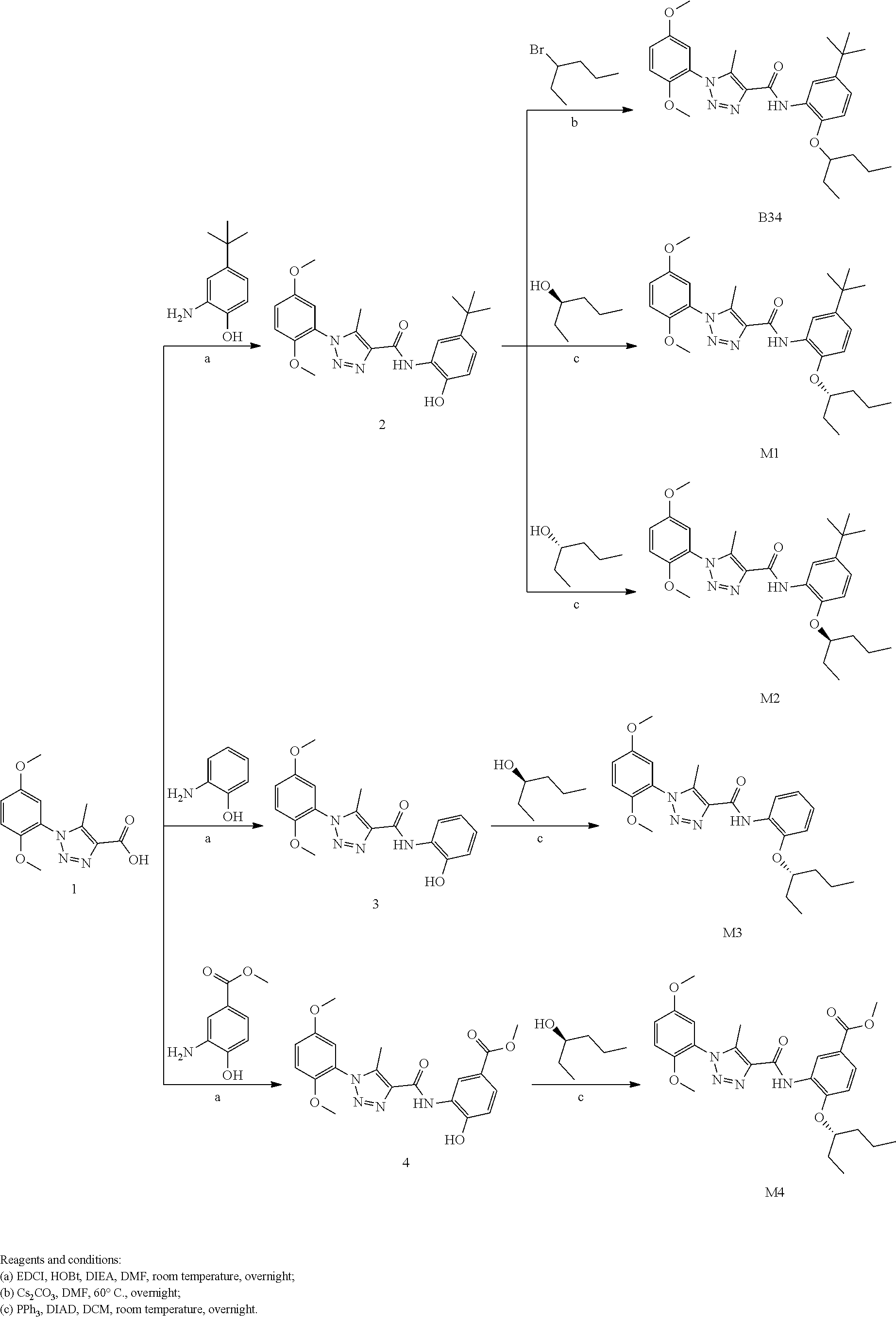

As shown above, compound 1 was coupled with the corresponding aniline in the presence of EDCI and HOBt to give compounds 2-4 (PMID: 33458521). The intermediate compounds 2 was then subjected to S_{N}2 nucleophilic substitutions with the corresponding alkyl halides to give compound B34. Compounds 2-4 were coupled with the respective (R)-pentan-2-ol or (S)-pentan-2-ol to generate the corresponding compounds M1, M2, M3, and M4 via a Mitsunobu reaction (PMID: 33689932).

i. N-(5-(Tert-Butyl)-2-(Hexan-3-yloxy)Phenyl)-1-(2,5-Dimethoxyphenyl)-5-Methyl-1H-1,2,3-Triazole-4-Carboxamide (B34)

2-amino-4-(tert-butyl)phenol (0.165 g, 1.000 mmol) was added to a solution of 1-(2,5-dimethoxyphenyl)-5-methyl-1H-1,2,3-triazole-4-carboxylic acid (1, 0.263 g, 1 mmol), EDCI (0.288 g, 1.5 mmol), HOBt (wetted with not less than 20% by weight of water, 0.203 g, 1.2 mmol), and DIEA (0.258 g, 2.000 mmol) in DMF (5 mL). The resulting mixture was stirred at room temperature overnight, then it was diluted with water (50 mL) and extracted with EtOAc (50 mL×2). The combined organic phase was washed with saturated aq. NaHCO₃, water, and brine, then dried with MgSO₄ and concentrated. The residue was purified by flash chromatography (0% to 100% EtOAc in hexane) to give compound 2 as a white solid (181.8 mg, 56% yield). ¹H NMR (400 MHz, DMSO-d₆) δ 10.01 (s, 1H), 9.61 (s, 1H), 8.33 (d, J=2.4 Hz, 1H), 7.29 (d, J=9.2 Hz, 1H), 7.22 (dd, J=9.1, 3.0 Hz, 1H), 7.16 (d, J=3.0 Hz, 1H), 6.99 (dd, J=8.4, 2.4 Hz, 1H), 6.86 (d, J=8.5 Hz, 1H), 3.78 (s, 3H), 3.76 (s, 3H), 2.41 (s, 3H), 1.27 (s, 9H). ¹³C NMR (101 MHz, DMSO-d₆) δ 158.45, 153.06, 147.65, 144.15, 141.51, 138.80, 137.30, 125.64, 123.61, 120.65, 117.44, 116.80, 114.28, 113.98, 113.89, 56.32, 55.85, 33.91, 31.39, 8.72.

To a solution of N-(5-(tert-butyl)-2-hydroxyphenyl)-1-(2,5-dimethoxyphenyl)-5-methyl-1H-1,2,3-triazole-4-carboxamide (2, 100 mg, 0.244 mmol) in DMF 5 mL was added Cs₂CO₃ (175 mg, 0.536 mmol) and 3-bromohexane (48.3 mg, 0.292 mmol). The suspension was stirred at 60° C. for overnight. The reaction mixture was then diluted with water (50 mL) and extracted with EtOAc (50 mL×2). The EtOAc layer was washed with water, dried with anhydrous Na₂SO₄, and concentrated. The residue was purified by silica gel chromatography (0% to 100% EA in hexane) to give product B34 as a white solid (95.3 mg, 79% yield, 99.24% purity). ¹H NMR (500 MHz, CDCl₃) δ 9.88 (s, 1H), 8.66 (d, J=2.4 Hz, 1H), 7.08 (dd, J=9.1, 3.0 Hz, 1H), 7.07-7.01 (m, 2H), 6.97 (d, J=3.0 Hz, 1H), 6.84 (d, J=8.6 Hz, 1H), 4.27 (p, J=5.8 Hz, 1H), 3.82 (s, 3H), 3.76 (s, 3H), 2.54 (s, 3H), 1.82-1.63 (m, 4H), 1.53-1.42 (m, 2H), 1.36 (s, 9H), 1.01 (t, J=7.5 Hz, 3H), 0.94 (t, J=7.4 Hz, 3H). ¹³C NMR (126 MHz, CDCl₃) δ 159.42, 153.68, 148.01, 145.03, 143.60, 139.04, 138.56, 128.36, 124.53, 120.05, 117.46, 117.01, 113.71, 113.30, 112.03, 80.27, 56.34, 56.01, 35.51, 34.45, 31.60, 26.57, 18.69, 14.23, 9.58, 9.32. ESI-TOF HRMS: m/z 495.2967 (C₂₈H₃₈N₄O₄+H⁺ requires 495.2966).

ii. (S)—N-(5-(Tert-Butyl)-2-(Hexan-3-yloxy)Phenyl)-1-(2,5-Dimethoxyphenyl)-5-Methyl-1H-1,2,3-Triazole-4-Carboxamide (M1)

N-(5-(tert-butyl)-2-hydroxyphenyl)-1-(2,5-dimethoxyphenyl)-5-methyl-1H-1,2,3-triazole-4-carboxamide (2, 100 mg, 0.244 mmol), (R)-hexan-3-ol (45.6 μl, 0.365 mmol), and PPh₃ (83 mg, 0.317 mmol) were added to dry DCM (10 mL) in a dried 25-mL round-bottom flask charged with a stir bar under an inert atmosphere. To the solution was added diisopropyl diazene-1,2-dicarboxylate (DIAD, 72.0 μl, 0.365 mmol). The reaction mixture was stirred at room temperature overnight. Then the reaction mixture quenched by adding sodium hydroxide solution (0.5 N, 10 mL) and extracted with EtOAc (25 mL×2). The combined organic layers were washed with water and brine, dried over anhydrous MgSO₄, and concentrated. The residue was purified by silica gel chromatography (0% to 100% EtOAc in hexane) to give product (S)—N-(5-(tert-butyl)-2-(hexan-3-yloxy)phenyl)-1-(2,5-dimethoxyphenyl)-5-methyl-1H-1,2,3-triazole-4-carboxamide as a white solid (83.2 mg, 71% yield, 99.57% purity, % ee=100%). ¹H NMR (500 MHz, DMSO-d₆) δ 9.70 (s, 1H), 8.50 (d, J=2.4 Hz, 1H), 7.29 (d, J=9.2 Hz, 1H), 7.22 (dd, J=9.2, 3.1 Hz, 1H), 7.17 (d, J=3.0 Hz, 1H), 7.08 (dd, J=8.6, 2.4 Hz, 1H), 7.03 (d, J=8.7 Hz, 1H), 4.40 (p, J=5.7 Hz, 1H), 3.78 (s, 3H), 3.76 (s, 3H), 2.41 (s, 3H), 1.74-1.60 (m, 4H), 1.48-1.38 (m, 2H), 1.29 (s, 9H), 0.95 (t, J=7.4 Hz, 3H), 0.91 (t, J=7.4 Hz, 3H). ¹³C NMR (126 MHz, DMSO-d₆) δ 158.84, 153.55, 148.10, 145.02, 143.31, 139.38, 137.80, 127.99, 124.06, 120.93, 117.99, 116.66, 114.43, 114.37, 113.22, 79.76, 56.82, 56.34, 35.45, 34.53, 31.81, 26.47, 18.47, 14.53, 9.59, 9.24. ESI-TOF HRMS: m/z 495.2956 (C₂₈H₃₈N₄O₄+H⁺ requires 495.2966).

iii. (R)—N-(5-(Tert-Butyl)-2-(Hexan-3-yloxy)Phenyl)-1-(2,5-Dimethoxyphenyl)-5-Methyl-1H-1,2,3-Triazole-4-Carboxamide (M2)

This compound was synthesized by using a procedure similar to that described for compound M1, employing compound 2 and (S)-hexan-3-ol, to give a white solid (98.8 mg, 82% yield, 100% purity, % ee=100%). ¹H NMR (500 MHz, DMSO-d₆) δ 9.70 (s, 1H), 8.50 (d, J=2.4 Hz, 1H), 7.29 (d, J=9.2 Hz, 1H), 7.22 (dd, J=9.2, 3.1 Hz, 1H), 7.17 (d, J=3.0 Hz, 1H), 7.08 (dd, J=8.7, 2.4 Hz, 1H), 7.03 (d, J=8.7 Hz, 1H), 4.40 (p, J=5.8 Hz, 1H), 3.78 (s, 3H), 3.76 (s, 3H), 2.41 (s, 3H), 1.78-1.60 (m, 4H), 1.50-1.38 (m, 2H), 1.29 (s, 9H), 0.95 (t, J=7.4 Hz, 3H), 0.91 (t, J=7.4 Hz, 3H). ¹³C NMR (126 MHz, DMSO-d₆) δ 158.84, 153.55, 148.10, 145.02, 143.31, 139.38, 137.80, 127.99, 124.06, 120.93, 117.99, 116.66, 114.43, 114.37, 113.22, 79.76, 56.82, 56.34, 35.45, 34.53, 31.81, 26.47, 18.47, 14.53, 9.59, 9.24. ESI-TOF HRMS: m/z 495.2980 (C₂₈H₃₈N₄O₄+H⁺ requires 495.2966).

iv. (S)-1-(2,5-Dimethoxyphenyl)-N-(2-(Hexan-3-yloxy)Phenyl)-5-Methyl-1H-1,2,3-Triazole-4-Carboxamide (M3)

Compound 3 was synthesized by using a procedure similar to that described for compound 2, employing compound 1 and 2-aminophenol, to give a white solid (369.2 mg, 55% yield). ¹H NMR (500 MHz, DMSO-d₆) δ 10.27 (s, 1H), 9.61 (s, 1H), 8.22 (dd, J=8.0, 1.4 Hz, 1H), 7.29 (d, J=9.2 Hz, 1H), 7.22 (dd, J=9.1, 3.0 Hz, 1H), 7.15 (d, J=3.0 Hz, 1H), 7.01-6.90 (m. 2H), 6.84 (ddd, J=8.5, 6.8, 2.1 Hz, 1H), 3.78 (s, 3H), 3.75 (s, 3H), 2.40 (s, 3H). ¹³C NMR (126 MHz, DMSO-d₆) δ 157.85, 152.47, 147.08, 145.89, 138.28, 136.66, 125.56, 123.51, 123.00, 119.07, 118.65, 116.87, 114.22, 113.41, 113.30, 55.74, 55.27, 8.14.

Compound M3 was synthesized by using a procedure similar to that described for compound M1, employing compound 3 and (R)-hexan-3-ol, to give a white solid (140.2 mg, 71% yield, 100% purity). ¹H NMR (500 MHz, DMSO-d₆) δ 9.73 (s, 1H), 8.37 (d, J=8.0 Hz, 1H), 7.29 (d, J=9.1 Hz, 1H), 7.22 (dd, J=9.3, 3.0 Hz, 1H), 7.18-7.12 (m, 2H), 7.08 (t, J=7.9 Hz, 1H), 6.96 (t, J=7.7 Hz, 1H), 4.46 (p, J=5.7 Hz, 1H), 3.78 (s, 3H), 3.76 (s, 3H), 2.41 (s, 3H), 1.69 (tt, J=14.6, 6.9 Hz, 4H), 1.43 (dp, J=14.3, 6.9 Hz, 2H), 0.96 (t, J=7.4 Hz, 3H), 0.91 (t, J=7.4 Hz, 3H). ¹³C NMR (126 MHz, DMSO-d₆) δ 158.84, 153.55, 148.11, 147.25, 139.44, 137.73, 128.47, 124.43, 124.05, 121.06, 119.47, 117.99, 114.46, 114.37, 113.86, 79.78, 56.81, 56.34, 35.39, 26.43, 18.43, 14.52, 9.57, 9.23. ESI-TOF HRMS: m/z 439.2345 (C₂₄H₃₀N₄O₄+H⁺ requires 439.2340).

v. Methyl (S)-3-(1-(2,5-Dimethoxyphenyl)-5-Methyl-1H-1,2,3-Triazole-4-Carboxamido)-4-(Hexan-3-yloxy)Benzoate (M4)

Compound 4 was synthesized by using a procedure similar to that described for compound 2, employing compound 1 and methyl 3-amino-4-hydroxybenzoate, to give a white solid (2.34 g, 50% yield). ¹H NMR (500 MHz, DMSO-d₆) δ 11.35 (s, 1H), 9.63 (s, 1H), 8.91 (d, J=2.2 Hz, 1H), 7.65 (dd, J=8.4, 2.2 Hz, 1H), 7.29 (d, J=9.2 Hz, 1H), 7.22 (dd, J=9.2, 3.1 Hz, 1H), 7.16 (d, J=3.1 Hz, 1H), 7.04 (d, J=8.4 Hz, 1H), 3.82 (s, 3H), 3.78 (s, 3H), 3.76 (s, 3H), 2.42 (s, 3H). ¹³C NMR (126 MHz, DMSO-d₆) δ 165.49, 158.02, 152.49, 150.60, 147.09, 138.53, 136.48, 125.58, 125.47, 122.97, 119.94, 119.76, 116.92, 113.88, 113.43, 113.32, 55.76, 55.28, 51.21, 8.15.

Compound M4 was synthesized by using a procedure similar to that described for compound M1, employing compound 4 and (R)-hexan-3-ol, to give a white solid (113.0 mg, 94% yield, 100% purity). ¹H NMR (500 MHz, DMSO-d₆) δ 9.75 (s, 1H), 9.01 (d, J=2.1 Hz, 1H), 7.74 (dd, J=8.6, 2.2 Hz, 1H), 7.31-7.26 (m, 2H), 7.23 (dd, J=9.1, 3.1 Hz, 1H), 7.17 (d, J=3.0 Hz, 1H), 4.62 (p, J=5.7 Hz, 1H), 3.85 (s, 3H), 3.78 (s, 3H), 3.76 (s, 3H), 2.42 (s, 3H), 1.82-1.61 (m, 4H), 1.54-1.34 (m, 2H), 0.96 (t, J=7.4 Hz, 3H), 0.91 (t, J=7.3 Hz, 3H). ¹³C NMR (126 MHz, DMSO-d₆) δ 165.29, 157.98, 152.47, 150.03, 147.03, 138.64, 136.43, 127.00, 125.29, 122.91, 121.00, 119.10, 116.95, 113.38, 113.31, 111.98, 79.07, 55.75, 55.26, 51.39, 34.13, 25.25, 17.26, 13.40, 8.38, 8.14. ESI-TOF HRMS: m/z 497.2387 (C₂₆H₃₂N₄O₆+H⁺ requires 497.2395).

ttttttt. Preparation of M5, M6, M7, and M8

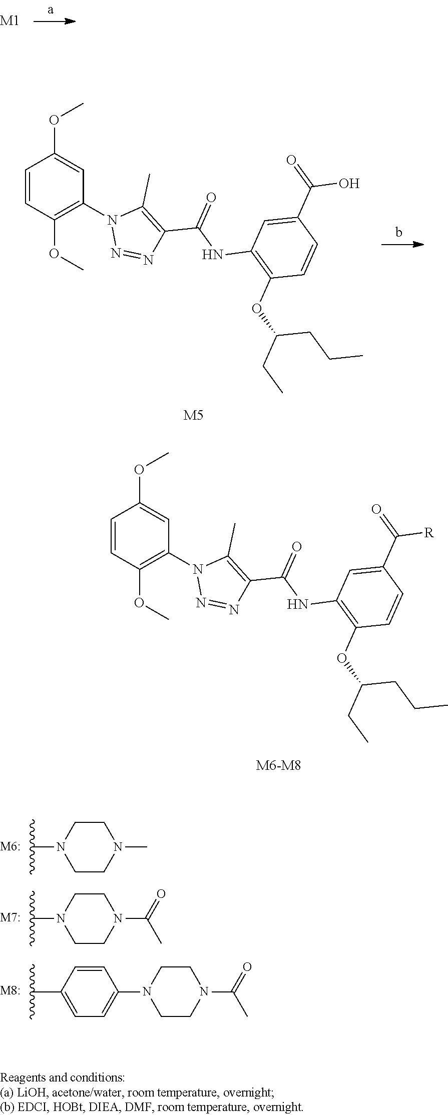

### i. (S)-3-(1-(2,5-Dimethoxyphenyl)-5-Methyl-1H-1,2,3-Triazole-4-Carboxamido)-4-(Hexan-3-yloxy)Benzoic Acid (M5)

To a solution of methyl (S)-3-(1-(2,5-dimethoxyphenyl)-5-methyl-1H-1,2,3-triazole-4-carboxamido)-4-(hexan-3-yloxy)benzoate (500 mg, 1.007 mmol) in acetone/water 10/10 mL was added lithium hydroxide (241 mg, 10.07 mmol) at room temperature. The suspension was stirred for overnight. The reaction mixture was adjusted pH by TN HCl to <4 and extracted with EtOAc (50 mL×2). The EtOAc layer was washed with water, dried with anhydrous MgSO₄, and concentrated. The residue was solid and washed by 0° C. cold Et₂O to give product as a white solid (385.6 mg, 79% yield, 100% purity). ¹H NMR (500 MHz, DMSO-d₆) δ 12.72 (s, 1H), 9.73 (s, 1H), 8.98 (d, J=2.1 Hz, 1H), 7.71 (dd, J=8.6, 2.2 Hz, 1H), 7.29 (d, J=9.2 Hz, 1H), 7.26-7.21 (m, 2H), 7.17 (d, J=3.0 Hz, 1H), 4.60 (t, J=5.8 Hz, 1H), 3.77 (s, 3H), 3.76 (s, 3H), 2.41 (s, 3H), 1.81-1.64 (m, 4H), 1.53-1.34 (m, 2H), 0.96 (t, J=7.4 Hz, 3H), 0.91 (t, J=7.3 Hz, 3H). ¹³C NMR (126 MHz, DMSO-d₆) δ 166.38, 157.93, 152.47, 149.76, 147.04, 138.58, 136.49, 126.86, 125.35, 122.93, 122.17, 119.48, 116.94, 113.38, 113.30, 111.81, 78.99, 55.74, 55.26, 34.16, 25.27, 17.28, 13.40, 8.40, 8.13.

ESI-TOF HRMS: m/z 483.2250 (C₂₅H₃₀N₄O₆+H⁺ requires 483.2238).

### ii. (S)-1-(2,5-Dimethoxyphenyl)-N-(2-(Hexan-3-Yloxy)-5-(4-Methylpiperazine-1-Carbonyl)Phenyl)-5-Methyl-1H-1,2,3-Triazole-4-Carboxamide (M6)

To a solution of (S)-3-(1-(2,5-dimethoxyphenyl)-5-methyl-1H-1,2,3-triazole-4-carboxamido)-4-(hexan-3-yloxy)benzoic acid (50 mg, 0.104 mmol) in DMSO 2 mL was added 1-methylpiperazine (31.2 mg, 0.311 mmol), DIEA (40.2 mg, 0.311 mmol), HOBt (wetted with not less than 20% by weight of water, 16.80 mg, 0.124 mmol) and EDCI (29.8 mg, 0.155 mmol). The resulting mixture was stirred at room temperature overnight. The reaction mixture was purified by silica gel chromatography (0% to 100% acetonitrile in water) to give product a white solid (31.4 mg, 54% yield, 96.5% purity). ¹H NMR (500 MHz, DMSO-d₆) δ 9.75 (s, 1H), 8.44 (d, J=2.0 Hz, 1H), 7.29 (d, J=9.2 Hz, 1H), 7.22 (dd, J=9.2, 3.0 Hz, 1H), 7.19 (d, J=8.5 Hz, 1H), 7.17 (d, J=3.0 Hz, 1H), 7.13 (dd, J=8.4, 2.1 Hz, 1H), 4.54 (p, J=5.8 Hz, 1H), 3.77 (s, 3H), 3.76 (s, 3H), 3.59-3.47 (m, 4H), 2.41 (s, 3H), 2.36-2.24 (m, 4H), 2.20 (s, 3H), 1.79-1.60 (m, 4H), 1.53-1.34 (m, 2H), 0.96 (t, J=7.4 Hz, 3H), 0.91 (t, J=7.3 Hz, 3H). ¹³C NMR (126 MHz, DMSO-d₆) δ 169.23, 159.03, 153.55, 148.09, 139.63, 137.56, 128.24, 127.98, 124.00, 123.63, 118.54, 118.00, 114.44, 114.38, 113.07, 79.96, 56.81, 56.33, 55.06, 55.04, 46.09, 35.30, 26.37, 18.40, 14.50, 9.52, 9.23. ESI-TOF HRMS: m/z 565.3158 (C₃₀H₄₀N₆O₅+H⁺ requires 565.3133).

### iii. (S)—N-(5-(4-Acetylpiperazine-1-Carbonyl)-2-(Hexan-3-yloxy)Phenyl)-1-(2,5-Dimethoxyphenyl)-5-Methyl-1H-1,2,3-Triazole-4-Carboxamide (M7)

Compound M7 was synthesized by using a procedure similar to that described for compound M6, employing (S)-3-(1-(2,5-dimethoxyphenyl)-5-methyl-1H-1,2,3-triazole-4-carboxamido)-4-(hexan-3-yloxy)benzoic acid and 1-(piperazin-1-yl)ethan-1-one, to give a white solid (42.9 mg, 70% yield, 100% purity). ¹H NMR (500 MHz, DMSO-d₆) δ 9.76 (s, 1H), 8.48 (s, 1H), 7.29 (d, J=9.2 Hz, 1H), 7.24-7.21 (m, 1H), 7.21-7.16 (m, 3H), 4.55 (p, J=5.9 Hz, 1H), 3.77 (s, 3H), 3.76 (s, 3H), 3.60-3.45 (m, 8H), 2.41 (s, 3H), 2.03 (s, 3H), 1.71 (tdd, J=14.9, 10.9, 6.4 Hz, 4H), 1.52-1.37 (m, 2H), 0.97 (t, J=7.4 Hz, 3H), 0.91 (t, J=7.3 Hz, 3H). ¹³C NMR (126 MHz, DMSO-d₆) δ 169.49, 168.98, 159.04, 153.55, 148.26, 148.09, 139.64, 137.55, 128.01, 127.93, 124.00, 123.79, 118.73, 118.00, 114.44, 114.37, 113.06, 79.97, 56.81, 56.33, 46.07, 41.32, 35.30, 26.37, 21.73, 18.40, 14.50, 9.52, 9.22. ESI-TOF HRMS: m/z 593.3087 (C₃₁H₄₀N₆O₆+H⁺ requires 593.3082).

### iv. (S)—N-(5-((4-(4-Acetylpiperazin-1-yl)Phenyl)Carbamoyl)-2-(Hexan-3-yloxy)Phenyl)-1-(2,5-Dimethoxyphenyl)-5-Methyl-1H-1,2,3-Triazole-4-Carboxamide (M8)

Compound M8 was synthesized by using a procedure similar to that described for compound M6, employing (S)-3-(1-(2,5-dimethoxyphenyl)-5-methyl-1H-1,2,3-triazole-4-carboxamido)-4-(hexan-3-yloxy)benzoic acid and 1-(4-(4-aminophenyl)piperazin-1-yl)ethan-1-one, to give a white solid (44.6 mg, 63% yield, 100% purity). ¹H NMR (500 MHz, DMSO-d₆) δ 10.01 (s, 1H), 9.76 (s, 1H), 8.91 (s, 1H), 7.72 (d, J=8.5 Hz, 1H), 7.63 (d, J=8.4 Hz, 2H), 7.36-7.13 (m, 4H), 6.96 (d, J=8.7 Hz, 2H), 4.66-4.57 (m, 1H), 3.78 (s, 3H), 3.76 (s, 3H), 3.58 (q, J=5.3 Hz, 4H), 3.16-3.02 (m, 4H), 2.42 (s, 3H), 2.04 (s, 3H), 1.82-1.67 (m, 4H), 1.54-1.37 (m, 2H), 0.97 (t, J=7.3 Hz, 3H), 0.91 (t, J=7.5 Hz, 3H). ¹³C NMR (126 MHz, DMSO-d₆) δ 168.71, 165.17, 158.98, 153.55, 149.79, 148.11, 147.61, 139.62, 137.61, 132.25, 127.97, 127.94, 124.08, 124.02, 121.91, 119.51, 118.01, 116.62, 114.46, 114.38, 112.76, 79.95, 56.82, 56.34, 49.74, 49.30, 45.99, 41.16, 35.30, 26.40, 21.68, 18.37, 14.51, 9.50, 9.22. ESI-TOF HRMS: m/z 684.3527 (C₃₇H₄₅N₇O₆+H⁺ requires 684.3504).

uuuuuuu. Preparation of M9, M10, M11, and M12

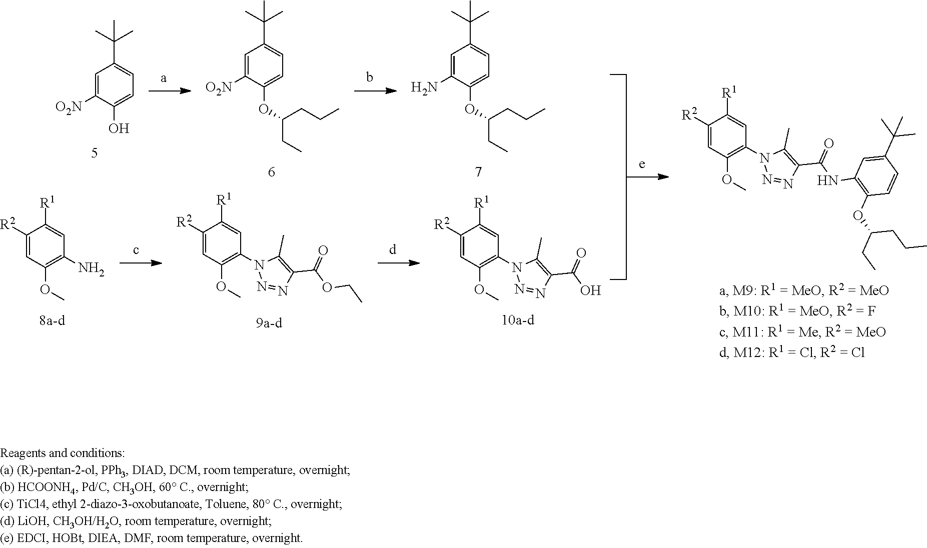

4-(tert-butyl)-2-nitrophenol (5) was coupled with the (R)-pentan-2-ol to generate (S)-4-(tert-butyl)-1-(hexan-3-yloxy)-2-nitrobenzene (6) via a Mitsunobu reaction (PMID: 33689932). Then the nitro group of (S)-4-(tert-butyl)-1-(hexan-3-yloxy)-2-nitrobenzene (6) was reduced by using Pd/C and HCOONH₄ in MeOH to generate to generate (S)-5-(tert-butyl)-2-(hexan-3-yloxy)aniline (7) (PMID: 18909189). Substituted aniline compounds 8a-d were first converted to the corresponding triazole compounds 9a-d through Wolff 1,2,3-triazole synthesis (PMID: 22683921, JP2011098956). Then, compounds 9a-d hydrolysis by LiOH to get the corresponding acid (PMID: 25147150). Final, the corresponding acid were coupled with the (S)-5-(tert-butyl)-2-(hexan-3-yloxy)aniline (7) to generate the corresponding compounds M9, M10, M11, and M12.

### i. (S)-4-(Tert-Butyl)-1-(Hexan-3-Yloxy)-2-Nitrobenzene (6)

Compound 6 was synthesized by using a procedure similar to that described for compound M1, employing 4-(tert-butyl)-2-nitrophenol and (R)-hexan-3-ol, to give a yellow oil (2.93 g, 94% yield). ¹H NMR (500 MHz, DMSO-d₆) δ 7.73 (d, J=2.5 Hz, 1H), 7.60 (dd, J=8.9, 2.5 Hz, 1H), 7.27 (d, J=8.9 Hz, 1H), 4.49 (p, J=5.8 Hz, 1H), 1.72-1.48 (m, 4H), 1.43-1.27 (m, 2H), 1.27 (s, 9H), 0.94-0.81 (m, 6H). ¹³C NMR (126 MHz, DMSO-d₆) δ 149.31, 143.68, 141.00, 131.68, 122.05, 116.29, 80.49, 35.69, 34.93, 31.73, 26.75, 18.71, 14.82, 9.88.

### ii. (S)-5-(Tert-Butyl)-2-(Hexan-3-yloxy)Aniline (7)

Add 10% Pd/C (0.6 g) to a solution of (S)-4-(tert-butyl)-1-(hexan-3-yloxy)-2-nitrobenzene (2.93 g, 10.49 mmol) and ammonium formate (3.31 g, 52.4 mmol) in ethanol (75 mL). The reaction mixture was heated at reflux for 12 h. Then the reaction mixture was cool to 25° C., and filter through a plug of Celite. Wash the Celite with methanol (20 mL) and concentrate the filtrate. The residue was added saturation NaHCO₃ water solution (100 mL), and extracted with EtOAc (100 mL×2). The combined organic layers were washed with water and brine, dried over anhydrous MgSO₄, and concentrated. The residue was purified by silica gel chromatography (0% to 100% EtOAc in hexane) to give product (S)-5-(tert-butyl)-2-(hexan-3-yloxy)aniline as a red oil (1.13 g, 43% yield). ¹H NMR (500 MHz, DMSO-d₆) δ 6.74-6.60 (m, 2H), 6.54-6.45 (m, 1H), 4.47 (s, 2H), 4.11 (p, J=5.8 Hz, 1H), 1.64-1.47 (m, 4H), 1.46-1.29 (m, 2H), 1.20 (s, 9H), 0.99-0.74 (m, 6H). ¹³C NMR (126 MHz, DMSO-d₆) δ 143.29, 143.25, 138.20, 113.31, 113.23, 112.17, 78.84, 35.73, 34.12, 31.90, 26.59, 18.62, 14.59, 9.85.

### iii. Ethyl 5-Methyl-1-(2,4,5-Trimethoxyphenyl)-1H-1,2,3-Triazole-4-Carboxylate (9A)

To a solution of 2,4,5-trimethoxyaniline (0.630 g, 3.44 mmol) and ethyl 2-diazo-3-oxobutanoate (500 μL, 3.44 mmol) in toluene 5 mL and THF 3 mL was added titanium(IV) chloride (1.0 M solution in toluene, 5.16 mL, 5.16 mmol). The mixture was warmed to 75° C. and stirred for overnight. The reaction mixture was treated with 50 mL water and extracted with EA (2×50 mL). The combined organic layer was washed with brine (50 mL), dried over MgSO₄ and filtered. The filtrate was concentrated in vacuum, and then purified by silica gel column chromatography (0% to 100% EtOAc in hexane) to give product as a yellow solid (210.3 mg, 19% yield). ¹H NMR (500 MHz, DMSO-d₆) δ 7.09 (s, 1H), 6.95 (s, 1H), 4.34 (q, J=7.1 Hz, 2H), 3.90 (s, 3H), 3.77 (s, 3H), 3.73 (s, 3H), 2.31 (s, 3H), 1.33 (t, J=7.1 Hz, 3H). ¹³C NMR (126 MHz, DMSO-d₆) δ 160.53, 150.81, 147.65, 141.94, 140.17, 134.35, 113.81, 111.45, 97.76, 59.76, 55.96, 55.73, 55.49, 13.60, 8.57.

### iv. Ethyl 1-(4-Fluoro-2,5-Dimethoxyphenyl)-5-Methyl-1H-1,2,3-Triazole-4-Carboxylate (9B)

Compound 9b was synthesized by using a procedure similar to that described for compound 9a, employing 4-fluoro-2,5-dimethoxyaniline and ethyl 2-diazo-3-oxobutanoate, to give a white solid (315.3 mg, 30% yield). ¹H NMR (500 MHz, DMSO-d₆) δ 7.42 (d, J=5.6 Hz, 1H), 7.39 (d, J=9.6 Hz, 1H), 4.35 (q, J=7.1 Hz, 2H), 3.82 (s, 3H), 3.75 (s, 3H), 2.32 (s, 3H), 1.33 (t, J=7.1 Hz, 3H). ¹³C NMR (126 MHz, DMSO-d₆) δ 160.43, 153.32, 151.34, 147.49, 147.42, 140.33, 140.24, 140.22, 134.48, 118.03, 118.00, 113.87, 113.84, 102.06, 101.88, 59.84, 56.24, 56.23, 13.59, 8.50.

### v. Ethyl 1-(2,4-Dimethoxy-5-Methylphenyl)-5-Methyl-1H-1,2,3-Triazole-4-Carboxylate (9C)

Compound 9c was synthesized by using a procedure similar to that described for compound 9a, employing 2,4-dimethoxy-5-methylaniline and ethyl 2-diazo-3-oxobutanoate, to give a white solid (372.1 mg, 35% yield). ¹H NMR (500 MHz, DMSO-d₆) δ 7.22 (s, 1H), 6.86 (s, 1H), 4.33 (q, J=7.1 Hz, 2H), 3.93 (s, 3H), 3.81 (s, 3H), 2.29 (s, 3H), 2.13 (s, 3H), 1.33 (t, J=7.1 Hz, 3H). ¹³C NMR (126 MHz, DMSO-d₆) δ 160.54, 159.21, 152.38, 140.05, 134.36, 128.59, 117.24, 114.43, 95.77, 59.75, 55.59, 55.36, 14.33, 13.59, 8.54.

### vi. Ethyl 1-(4,5-Dichloro-2-Methoxyphenyl)-5-Methyl-1H-1,2,3-Triazole-4-Carboxylate (9D)

Compound 9d was synthesized by using a procedure similar to that described for compound 9a, employing 4,5-dichloro-2-methoxyaniline and ethyl 2-diazo-3-oxobutanoate, to give a white solid (411 mg, 36% yield). ¹H NMR (500 MHz, DMSO-d₆) δ 7.93 (s, 1H), 7.69 (s, 1H), 4.35 (q, J=7.1 Hz, 2H), 3.85 (s, 3H), 2.34 (s, 3H), 1.33 (t, J=7.1 Hz, 3H). ¹³C NMR (126 MHz, DMSO-d₆) δ 160.31, 152.67, 140.39, 134.63, 134.17, 129.21, 122.45, 121.99, 114.76, 59.92, 56.54, 13.57, 8.45.

### vii. (S)—N-(5-(Tert-Butyl)-2-(Hexan-3-yloxy)Phenyl)-5-Methyl-1-(2,4,5-Trimethoxyphenyl)-1H-1,2,3-Triazole-4-Carboxamide (M9)

To a solution of ethyl 5-methyl-1-(2,4,5-trimethoxyphenyl)-1H-1,2,3-triazole-4-carboxylate (9a, 210 mg, 0.654 mmol) in water 3 mL/DMSO 3 mL was added lithium hydroxide (78 mg, 3.27 mmol). The mixture was stirred at room temperature for overnight. The reaction mixture was diluted by NH₄C1 (1 N, 50 mL) and extracted with EA (2×50 mL). The combined organic layer was washed with brine (50 mL), dried over MgSO₄ and filtered. The filtrate was concentrated in vacuum, the residue without purified for next step, grey solid, 136.2 mg.

In a 50 mL round-bottom flask, 5-methyl-1-(2,4,5-trimethoxyphenyl)-1H-1,2,3-triazole-4-carboxylic acid (10a, 100 mg, 0.341 mmol) was dissolved in DMF (5 mL). (S)-5-(tert-butyl)-2-(hexan-3-yloxy)aniline (102 mg, 0.409 mmol), EDCI (98 mg, 0.511 mmol), HOBt (wetted with not less than 20% by weight of water, 69.1 mg, 0.409 mmol) and DIEA (122 μl, 0.682 mmol) were added to the reaction mixture. The mixture was stirred at room temperature for overnight. Then the reaction mixture was added EA (50 mL) and washed by water, brine. The organic phase was concentrated, and the residue was purified by flash chromatography (0-100% EtOAc in hexane) to give compound M9 as a white solid (129.7 mg, 72% yield, 100% purity). ¹H NMR (500 MHz, DMSO-d₆) δ 9.70 (s, 1H), 8.50 (d, J=2.3 Hz, 1H), 7.15 (s, 1H), 7.08 (dd, J=8.6, 2.4 Hz, 1H), 7.03 (d, J=8.7 Hz, 1H), 6.96 (s, 1H), 4.40 (p, J=5.8 Hz, 1H), 3.92 (s, 3H), 3.80 (s, 3H), 3.74 (s, 3H), 2.39 (s, 3H), 1.68 (dtd, J=16.4, 13.7, 7.5 Hz, 4H), 1.51-1.36 (m, 2H), 1.29 (s, 9H), 0.95 (t, J=7.4 Hz, 3H), 0.91 (t, J=7.3 Hz, 3H). ¹³C NMR (126 MHz, DMSO-d₆) δ 157.84, 150.82, 147.61, 143.90, 142.23, 141.95, 138.44, 136.59, 126.95, 119.80, 115.53, 113.84, 112.14, 111.46, 97.75, 78.67, 55.97, 55.73, 55.49, 34.37, 33.45, 30.74, 25.38, 17.38, 13.46, 8.50, 8.17. ESI-TOF HRMS: m/z 525.3069 (C₂₉H₄₀N₄O₅+H^{|} requires 525.3071).

### viii. (S)—N-(5-(Tert-Butyl)-2-(Hexan-3-yloxy)Phenyl)-1-(4-Fluoro-2,5-Dimethoxyphenyl)-5-Methyl-1H-1,2,3-Triazole-4-Carboxamide (M10)

Compound M10 was synthesized by using a procedure similar to that described for compound M9, employing ethyl 1-(4-fluoro-2,5-dimethoxyphenyl)-5-methyl-1H-1,2,3-triazole-4-carboxylate (9b) and (S)-5-(tert-butyl)-2-(hexan-3-yloxy)aniline, to give a white solid (143.6 mg, two steps 57% yield, 100% purity). ¹H NMR (500 MHz, DMSO-d₆) δ 9.70 (s, 1H), 8.49 (d, J=2.3 Hz, 1H), 7.47 (d, J=8.9 Hz, 1H), 7.41 (d, J=12.9 Hz, 1H), 7.08 (dd, J=8.7, 2.4 Hz, 1H), 7.03 (d, J=8.7 Hz, 1H), 4.40 (p, J=5.8 Hz, 1H), 3.83 (s, 3H), 3.77 (s, 3H), 2.41 (s, 3H), 1.75-1.59 (m, 4H), 1.50-1.36 (m, 2H), 1.29 (s, 9H), 0.95 (t, J=7.4 Hz, 3H), 0.90 (t, J=7.3 Hz, 3H). ¹³C NMR (126 MHz, DMSO-d₆) δ 157.72, 153.34, 151.36, 147.46, 147.39, 143.94, 142.24, 140.37, 140.28, 138.52, 136.72, 126.91, 119.88, 118.08, 118.06, 115.58, 113.90, 113.87, 112.15, 102.08, 101.89, 78.68, 56.25, 34.37, 33.46, 30.73, 25.38, 17.38, 13.46, 8.49, 8.11. ESI-TOF HRMS: m/z 513.2876 (C₂₈H₃₇FN₄O₄+H⁺ requires 513.2872).

### ix. (S)—N-(5-(Tert-Butyl)-2-(Hexan-3-yloxy)Phenyl)-1-(2,4-Dimethoxy-5-Methylphenyl)-5-Methyl-1H-1,2,3-Triazole-4-Carboxamide (M11)

Compound M11 was synthesized by using a procedure similar to that described for compound M9, employing ethyl 1-(2,4-dimethoxy-5-methylphenyl)-5-methyl-1H-1,2,3-triazole-4-carboxylate (9c) and (S)-5-(tert-butyl)-2-(hexan-3-yloxy)aniline, to give a white solid (104.9 mg, two steps 50% yield, 99.5% purity). ¹H NMR (500 MHz, DMSO-d₆) δ 9.69 (s, 1H), 8.49 (d, J=2.4 Hz, 1H), 7.27 (s, 1H), 7.08 (dd, J=8.6, 2.4 Hz, 1H), 7.02 (d, J=8.6 Hz, 1H), 6.88 (s, 1H), 4.39 (p, J=5.8 Hz, 1H), 3.94 (s, 3H), 3.83 (s, 3H), 2.38 (s, 3H), 2.14 (s, 3H), 1.76-1.59 (m, 4H), 1.51-1.36 (m, 2H), 1.28 (s, 9H), 0.95 (t, J=7.4 Hz, 3H), 0.90 (t, J=7.3 Hz, 3H). ¹³C NMR (126 MHz, DMSO-d₆) δ 159.23, 157.87, 152.40, 143.93, 142.23, 138.33, 136.62, 128.63, 126.96, 119.79, 117.25, 115.56, 114.47, 112.16, 95.79, 78.73, 55.61, 55.37, 34.39, 33.45, 30.73, 25.40, 17.39, 14.35, 13.45, 8.52, 8.14. ESI-TOF HRMS: m/z 509.3141 (C₂₉H₄₀N₄O₄+H⁺ requires 509.3122).

### x. (S)—N-(5-(Tert-Butyl)-2-(Hexan-3-yloxy)Phenyl)-1-(4,5-Dichloro-2-Methoxyphenyl)-5-Methyl-1H-1,2,3-Triazole-4-Carboxamide (M12)

Compound M12 was synthesized by using a procedure similar to that described for compound M9, employing ethyl 1-(4,5-dichloro-2-methoxyphenyl)-5-methyl-1H-1,2,3-triazole-4-carboxylate (9d) and (S)-5-(tert-butyl)-2-(hexan-3-yloxy)aniline, to give a white solid (143.6 mg, two steps 68% yield, 100% purity). ¹H NMR (500 MHz, DMSO-d₆) δ 9.69 (s, 1H), 8.48 (d, J=2.4 Hz, 1H), 7.98 (s, 1H), 7.71 (s, 1H), 7.09 (dd, J=8.6, 2.4 Hz, 1H), 7.03 (d, J=8.7 Hz, 1H), 4.40 (p, J=5.8 Hz, 1H), 3.87 (s, 3H), 2.43 (s, 3H), 1.79-1.57 (m, 4H), 1.51-1.32 (m, 2H), 1.28 (s, 9H), 0.95 (t, J=7.4 Hz, 3H), 0.90 (t, J=7.4 Hz, 3H). 13C NMR (126 MHz, DMSO-d₆) δ 157.57, 152.66, 143.98, 142.23, 138.70, 136.82, 134.20, 129.25, 126.83, 122.49, 122.01, 119.95, 115.63, 114.78, 112.15, 78.69, 56.56, 34.37, 33.45, 30.73, 25.37, 17.37, 13.45, 8.50, 8.06. ESI-TOF HRMS: m/z 533.2098 (C₂₇H₃₄C₁₂N₄O₃+H⁺ requires 533.2081).

### 2. Biological Evaluation of Exemplary Compounds

A list of exemplary compounds evaluated for PXR activity as described herein are shown in Table 1.

TABLE 1

| Compound | |
|---|---|
| No. | Structure |
| T0901317 | 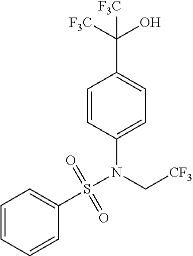 [SMILES: O=S(=O)(c1ccccc1)N(CC(F)(F)F)c1ccc(C(O)(C(F)(F)F)C(F)(F)F)cc1] |
| Rifampicin | 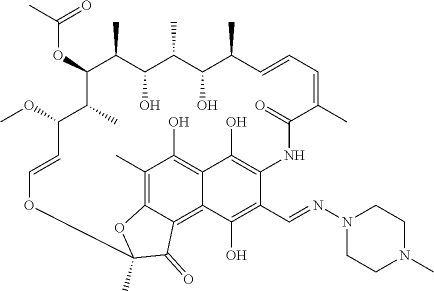 |
| SPA70 | 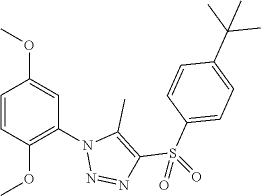 [SMILES: COc1ccc(OC)c(-n2nnc(S(=O)(=O)c3ccc(C(C)(C)C)cc3)c2C)c1] |
| A1 | 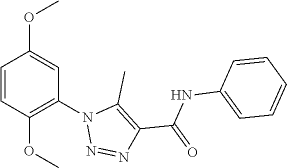 [SMILES: COc1ccc(OC)c(-n2nnc(C(=O)Nc3ccccc3)c2C)c1] |
| A2 | 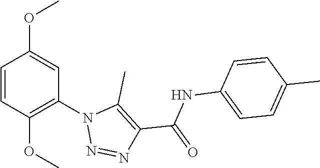 [SMILES: COc1ccc(OC)c(-n2nnc(C(=O)Nc3ccc(C)cc3)c2C)c1] |
| A3 | 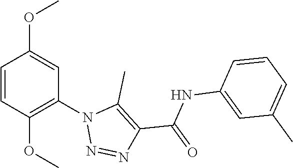 [SMILES: COc1ccc(OC)c(-n2nnc(C(=O)Nc3cccc(C)c3)c2C)c1] |
| A4 | 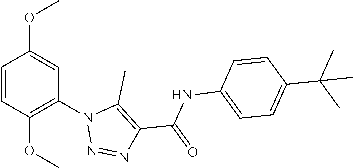 [SMILES: COc1ccc(OC)c(-n2nnc(C(=O)Nc3ccc(C(C)(C)C)cc3)c2C)c1] |
| A5 | 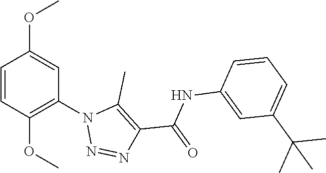 [SMILES: COc1ccc(OC)c(-n2nnc(C(=O)Nc3cccc(C(C)(C)C)c3)c2C)c1] |
| A6 | 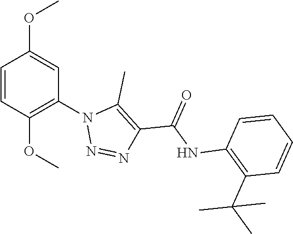 [SMILES: COc1ccc(OC)c(-n2nnc(C(=O)Nc3ccccc3C(C)(C)C)c2C)c1] |
| A7 | 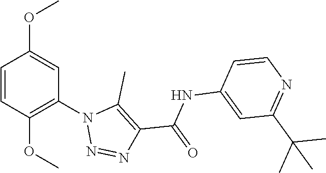 [SMILES: COc1ccc(OC)c(-n2nnc(C(=O)Nc3ccnc(C(C)(C)C)c3)c2C)c1] |
| A8 | 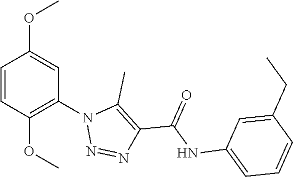 [SMILES: CCc1cccc(NC(=O)c2nnn(-c3cc(OC)ccc3OC)c2C)c1] |
| A9 | 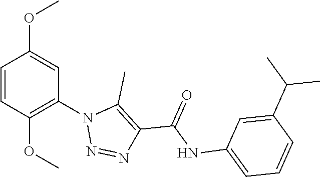 [SMILES: COc1ccc(OC)c(-n2nnc(C(=O)Nc3cccc(C(C)C)c3)c2C)c1] |
| A10 | 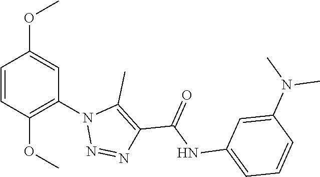 [SMILES: COc1ccc(OC)c(-n2nnc(C(=O)Nc3cccc(N(C)C)c3)c2C)c1] |
| A11 | 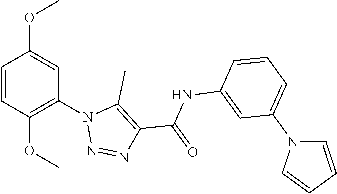 [SMILES: COc1ccc(OC)c(-n2nnc(C(=O)Nc3cccc(-n4cccc4)c3)c2C)c1] |
| A12 | 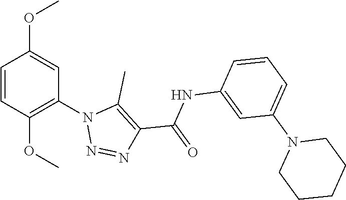 [SMILES: COc1ccc(OC)c(-n2nnc(C(=O)Nc3cccc(N4CCCCC4)c3)c2C)c1] |
| A13 | 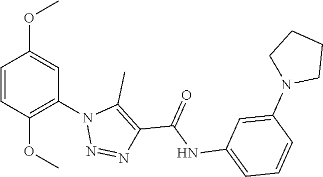 [SMILES: COc1ccc(OC)c(-n2nnc(C(=O)Nc3cccc(N4CCCC4)c3)c2C)c1] |
| A14 | 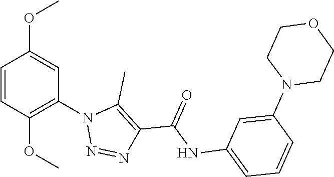 [SMILES: COc1ccc(OC)c(-n2nnc(C(=O)Nc3cccc(N4CCOCC4)c3)c2C)c1] |
| A15 | 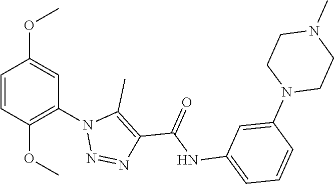 [SMILES: COc1ccc(OC)c(-n2nnc(C(=O)Nc3cccc(N4CCN(C)CC4)c3)c2C)c1] |
| A16 | 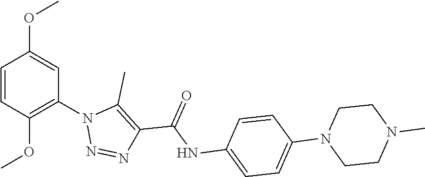 |
| A17 | 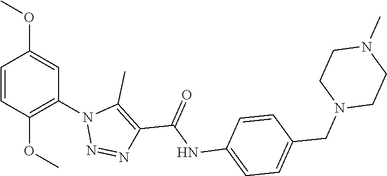 [SMILES: COc1ccc(OC)c(-n2nnc(C(=O)Nc3ccc(CN4CCN(C)CC4)cc3)c2C)c1] |
| A18 | 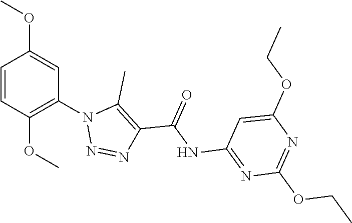 [SMILES: CCOc1cc(NC(=O)c2nnn(-c3cc(OC)ccc3OC)c2C)nc(OCC)n1] |
| A19 | 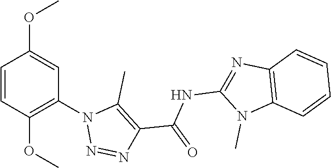 [SMILES: COc1ccc(OC)c(-n2nnc(C(=O)Nc3nc4ccccc4n3C)c2C)c1] |
| A20 | 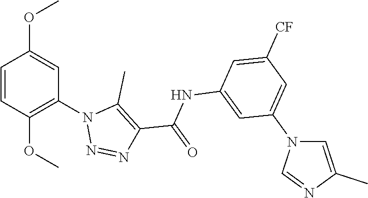 |
| A21 | 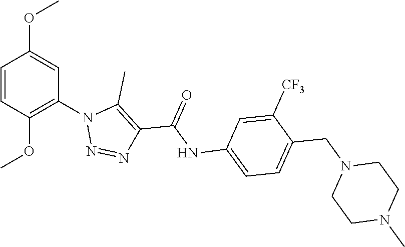 |
| A22 | 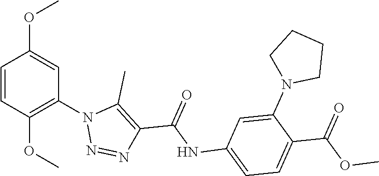 [SMILES: COC(=O)c1ccc(NC(=O)c2nnn(-c3cc(OC)ccc3OC)c2C)cc1N1CCCC1] |
| A23 | 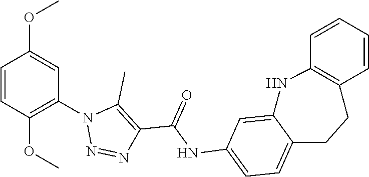 [SMILES: COc1ccc(OC)c(-n2nnc(C(=O)Nc3ccc4c(c3)Nc3ccccc3CC4)c2C)c1] |
| A24 | 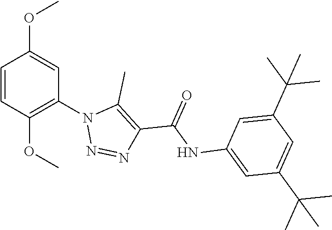 [SMILES: COc1ccc(OC)c(-n2nnc(C(=O)Nc3cc(C(C)(C)C)cc(C(C)(C)C)c3)c2C)c1] |
| A25 | 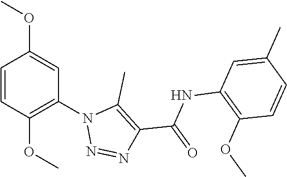 [SMILES: COc1ccc(OC)c(-n2nnc(C(=O)Nc3cc(C)ccc3OC)c2C)c1] |
| B1 | 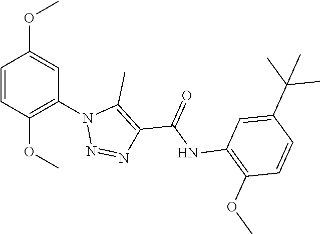 [SMILES: COc1ccc(OC)c(-n2nnc(C(=O)Nc3cc(C(C)(C)C)ccc3OC)c2C)c1] |
| B2 | 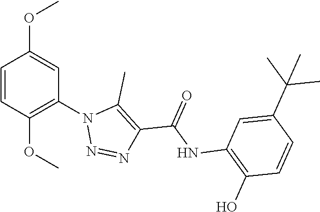 [SMILES: COc1ccc(OC)c(-n2nnc(C(=O)Nc3cc(C(C)(C)C)ccc3O)c2C)c1] |
| B3 | 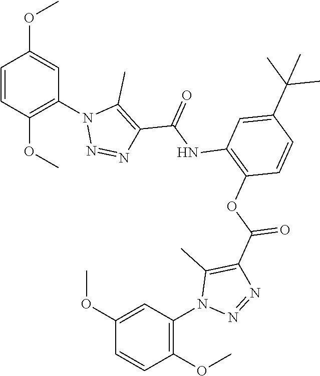 [SMILES: COc1ccc(OC)c(-n2nnc(C(=O)Nc3cc(C(C)(C)C)ccc3OC(=O)c3nnn(-c4cc(OC)ccc4OC)c3C)c2C)c1] |
| B4 | 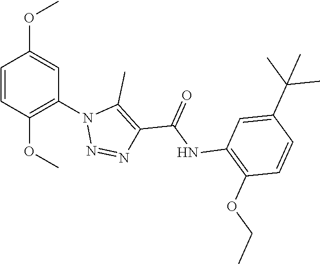 [SMILES: CCOc1ccc(C(C)(C)C)cc1NC(=O)c1nnn(-c2cc(OC)ccc2OC)c1C] |
| B5 | 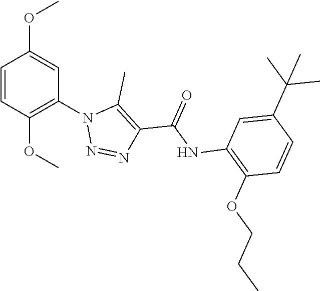 [SMILES: CCCOc1ccc(C(C)(C)C)cc1NC(=O)c1nnn(-c2cc(OC)ccc2OC)c1C] |
| B6 | 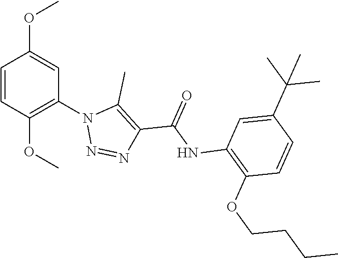 [SMILES: CCCCOc1ccc(C(C)(C)C)cc1NC(=O)c1nnn(-c2cc(OC)ccc2OC)c1C] |
| B7 | 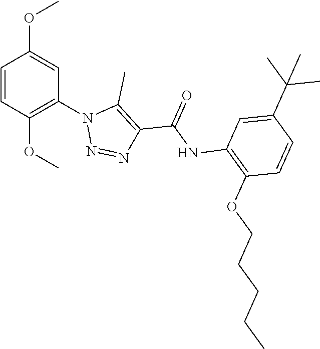 [SMILES: CCCCCOc1ccc(C(C)(C)C)cc1NC(=O)c1nnn(-c2cc(OC)ccc2OC)c1C] |
| B8 | 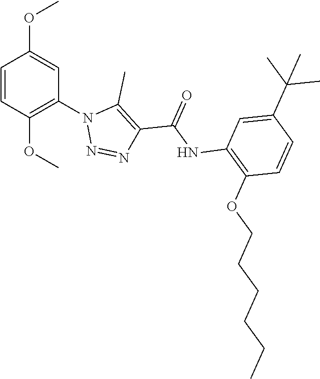 [SMILES: CCCCCCOc1ccc(C(C)(C)C)cc1NC(=O)c1nnn(-c2cc(OC)ccc2OC)c1C] |
| B9 | 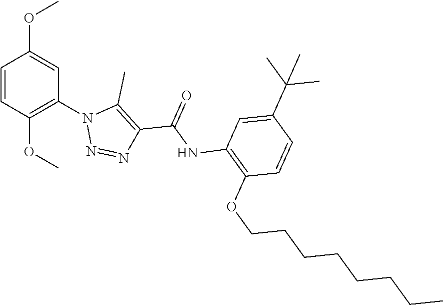 [SMILES: CCCCCCCCOc1ccc(C(C)(C)C)cc1NC(=O)c1nnn(-c2cc(OC)ccc2OC)c1C] |
| B10 | 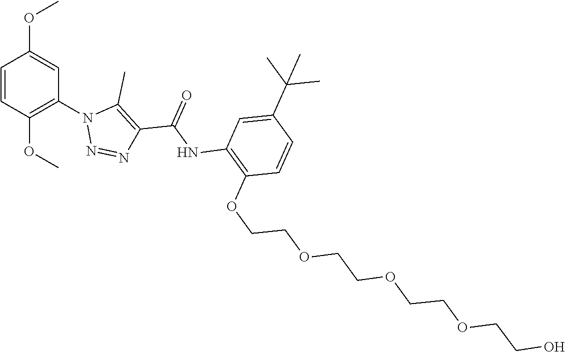 [SMILES: COc1ccc(OC)c(-n2nnc(C(=O)Nc3cc(C(C)(C)C)ccc3OCCOCCOCCOCCO)c2C)c1] |
| B11 | 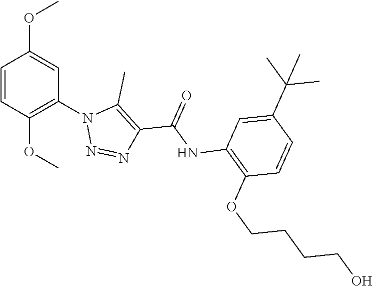 [SMILES: COc1ccc(OC)c(-n2nnc(C(=O)Nc3cc(C(C)(C)C)ccc3OCCCCO)c2C)c1] |
| B12 | 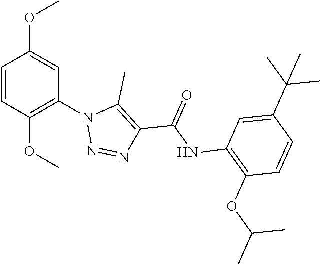 [SMILES: COc1ccc(OC)c(-n2nnc(C(=O)Nc3cc(C(C)(C)C)ccc3OC(C)C)c2C)c1] |
| B13 | 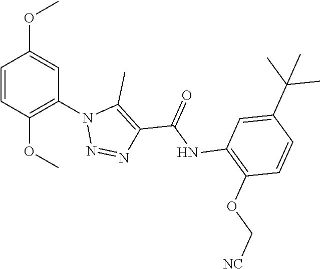 [SMILES: COc1ccc(OC)c(-n2nnc(C(=O)Nc3cc(C(C)(C)C)ccc3OCC#N)c2C)c1] |
| B14 | 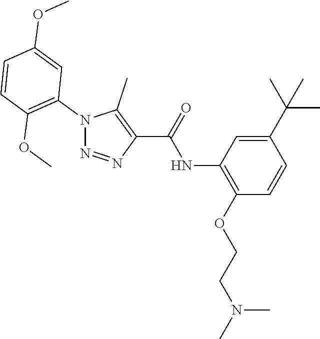 [SMILES: COc1ccc(OC)c(-n2nnc(C(=O)Nc3cc(C(C)(C)C)ccc3OCCN(C)C)c2C)c1] |
| B15 | 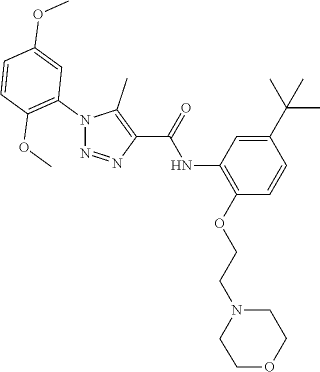 [SMILES: COc1ccc(OC)c(-n2nnc(C(=O)Nc3cc(C(C)(C)C)ccc3OCCN3CCOCC3)c2C)c1] |
| B16 | 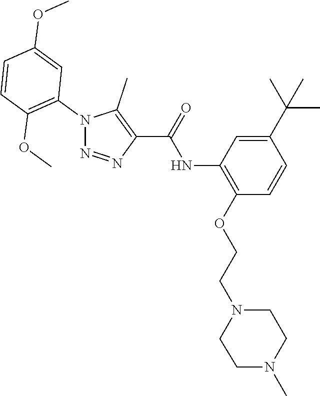 [SMILES: COc1ccc(OC)c(-n2nnc(C(=O)Nc3cc(C(C)(C)C)ccc3OCCN3CCN(C)CC3)c2C)c1] |
| B17 | 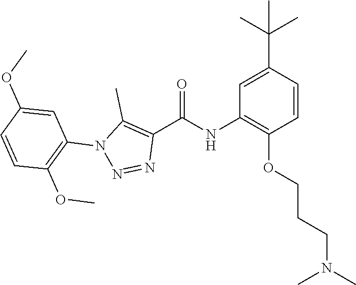 |
| B18 | 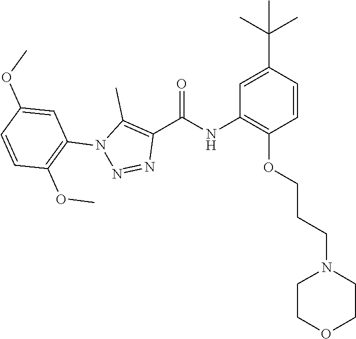 |
| B19 | 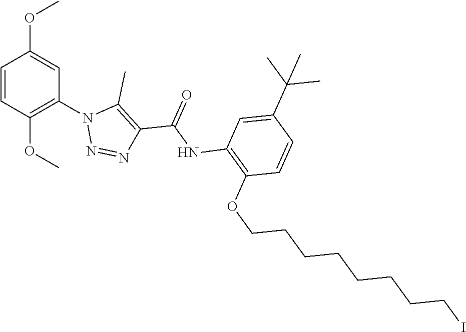 [SMILES: COc1ccc(OC)c(-n2nnc(C(=O)Nc3cc(C(C)(C)C)ccc3OCCCCCCCCI)c2C)c1] |
| B20 | 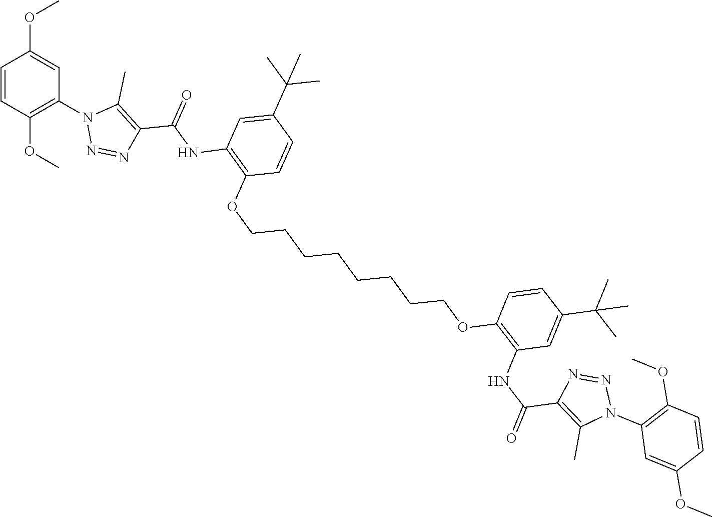 [SMILES: COc1ccc(OC)c(-n2nnc(C(=O)Nc3cc(C(C)(C)C)ccc3OCCCCCCCCOc3ccc(C(C)(C)C)cc3NC(=O)c3nnn(-c4cc(OC)ccc4OC)c3C)c2C)c1] |
| B21 | 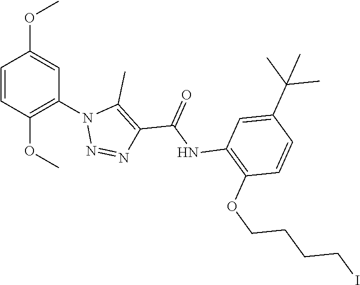 [SMILES: COc1ccc(OC)c(-n2nnc(C(=O)Nc3cc(C(C)(C)C)ccc3OCCCCI)c2C)c1] |
| B22 | 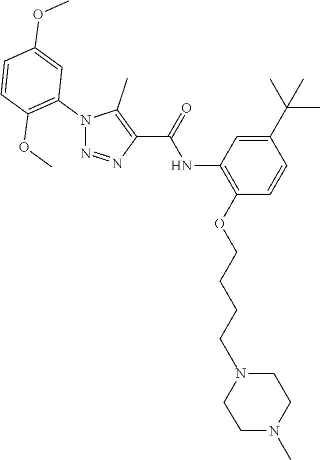 |
| B23 | 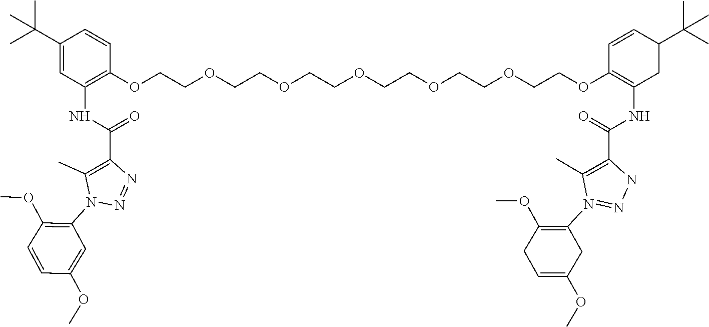 |
| B24 | 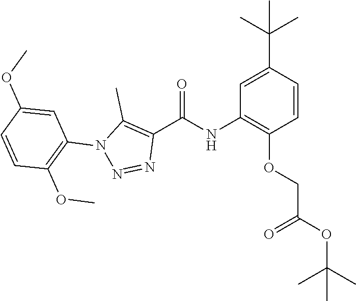 |
| B25 | 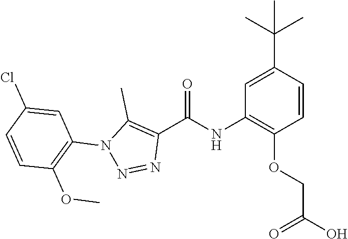 |
| B26 | 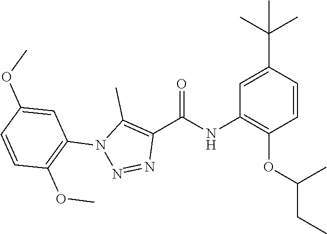 |
| B27 | 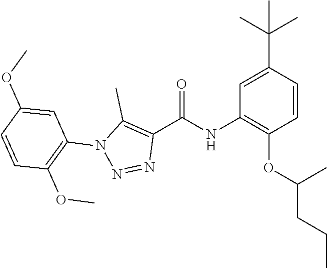 |
| B28 | 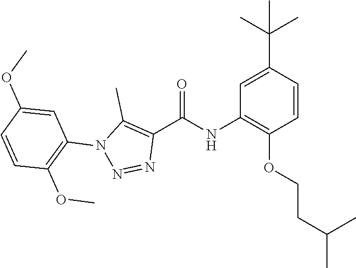 |
| B29 | 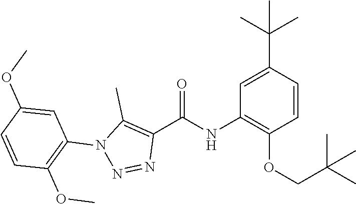 |
| B30 | 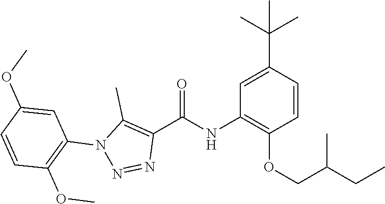 |
| B31 | 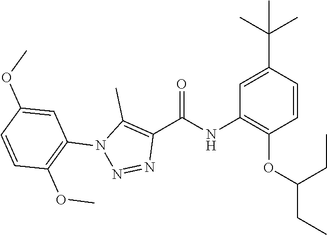 |
| B32 | 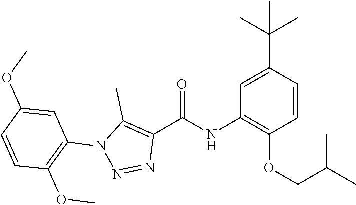 |
| B33 | 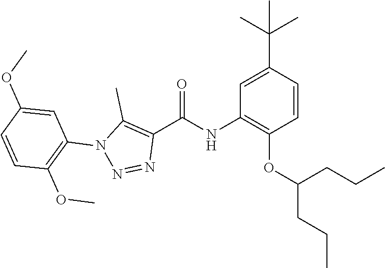 |
| B34 | 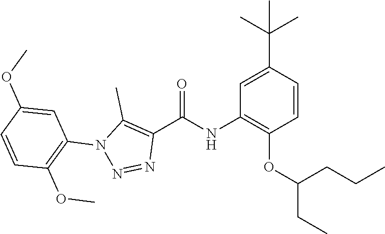 |
| B35 | 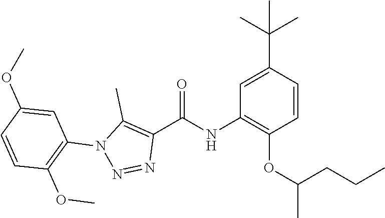 |
| B36 | 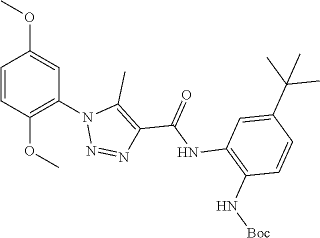 [SMILES: COc1ccc(OC)c(-n2nnc(C(=O)Nc3cc(C(C)(C)C)ccc3NC(=O)OC(C)(C)C)c2C)c1] |
| B37 | 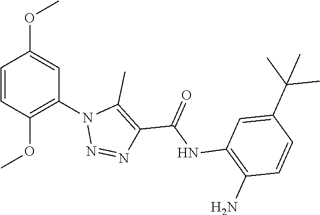 [SMILES: COc1ccc(OC)c(-n2nnc(C(=O)Nc3cc(C(C)(C)C)ccc3N)c2C)c1] |
| B38 | 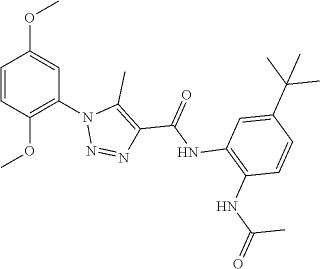 [SMILES: COc1ccc(OC)c(-n2nnc(C(=O)Nc3cc(C(C)(C)C)ccc3NC(C)=O)c2C)c1] |
| C1 | 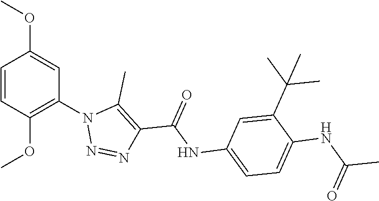 [SMILES: COc1ccc(OC)c(-n2nnc(C(=O)Nc3ccc(NC(C)=O)c(C(C)(C)C)c3)c2C)c1] |
| C2 | 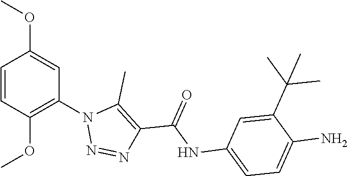 [SMILES: COc1ccc(OC)c(-n2nnc(C(=O)Nc3ccc(N)c(C(C)(C)C)c3)c2C)c1] |
| C3 | 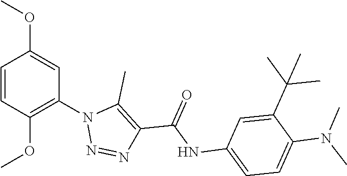 [SMILES: COc1ccc(OC)c(-n2nnc(C(=O)Nc3ccc(N(C)C)c(C(C)(C)C)c3)c2C)c1] |
| C4 | 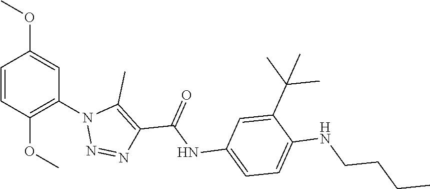 [SMILES: CCCCNc1ccc(NC(=O)c2nnn(-c3cc(OC)ccc3OC)c2C)cc1C(C)(C)C] |
| C5 | 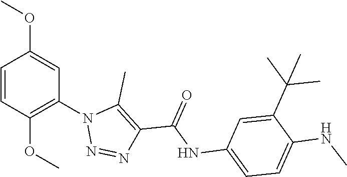 [SMILES: CNc1ccc(NC(=O)c2nnn(-c3cc(OC)ccc3OC)c2C)cc1C(C)(C)C] |
| C6 | 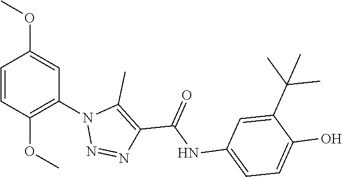 [SMILES: COc1ccc(OC)c(-n2nnc(C(=O)Nc3ccc(O)c(C(C)(C)C)c3)c2C)c1] |
| C7 | 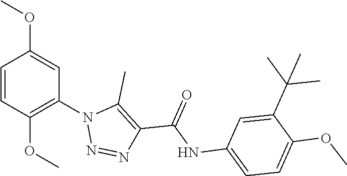 [SMILES: COc1ccc(OC)c(-n2nnc(C(=O)Nc3ccc(OC)c(C(C)(C)C)c3)c2C)c1] |
| C8 | 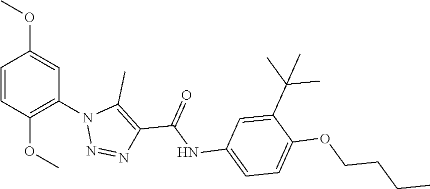 [SMILES: CCCCOc1ccc(NC(=O)c2nnn(-c3cc(OC)ccc3OC)c2C)cc1C(C)(C)C] |
| D1 | 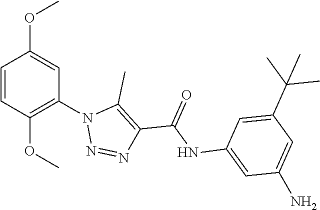 [SMILES: COc1ccc(OC)c(-n2nnc(C(=O)Nc3cc(N)cc(C(C)(C)C)c3)c2C)c1] |
| D2 | 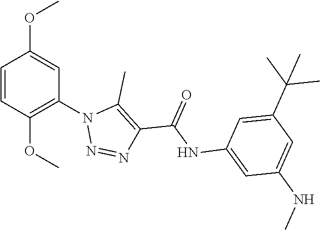 [SMILES: CNc1cc(NC(=O)c2nnn(-c3cc(OC)ccc3OC)c2C)cc(C(C)(C)C)c1] |
| D3 | 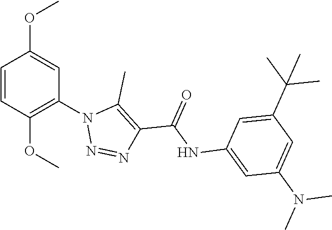 [SMILES: COc1ccc(OC)c(-n2nnc(C(=O)Nc3cc(N(C)C)cc(C(C)(C)C)c3)c2C)c1] |
| D4 | 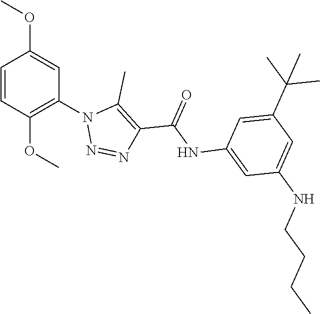 [SMILES: CCCCNc1cc(NC(=O)c2nnn(-c3cc(OC)ccc3OC)c2C)cc(C(C)(C)C)c1] |
| D5 | 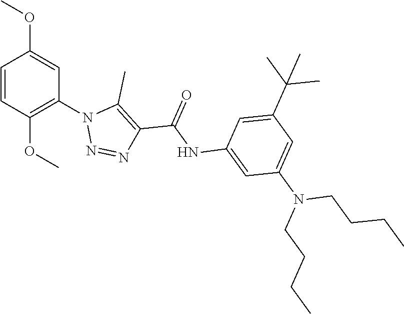 [SMILES: CCCCN(CCCC)c1cc(NC(=O)c2nnn(-c3cc(OC)ccc3OC)c2C)cc(C(C)(C)C)c1] |
| D6 | 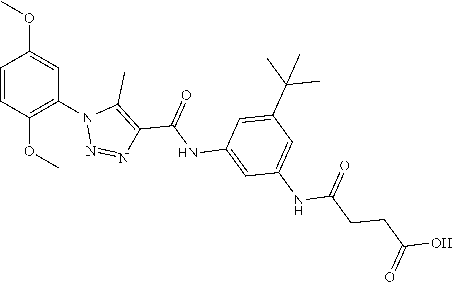 [SMILES: COc1ccc(OC)c(-n2nnc(C(=O)Nc3cc(NC(=O)CCC(=O)O)cc(C(C)(C)C)c3)c2C)c1] |
| D7 | 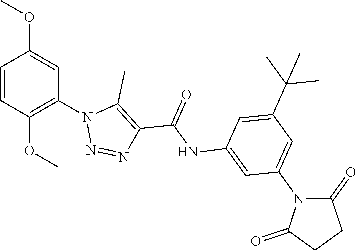 [SMILES: COc1ccc(OC)c(-n2nnc(C(=O)Nc3cc(N4C(=O)CCC4=O)cc(C(C)(C)C)c3)c2C)c1] |
| D8 | 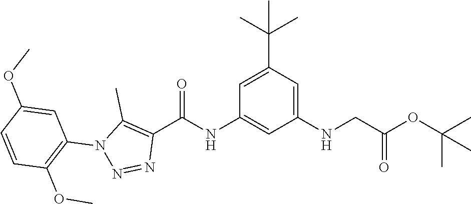 [SMILES: COc1ccc(OC)c(-n2nnc(C(=O)Nc3cc(NCC(=O)OC(C)(C)C)cc(C(C)(C)C)c3)c2C)c1] |
| D9 | 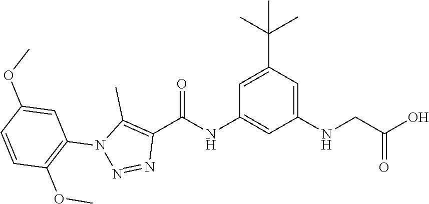 [SMILES: COc1ccc(OC)c(-n2nnc(C(=O)Nc3cc(NCC(=O)O)cc(C(C)(C)C)c3)c2C)c1] |
| D10 | 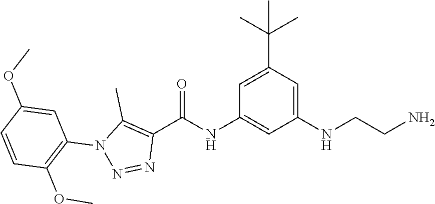 [SMILES: COc1ccc(OC)c(-n2nnc(C(=O)Nc3cc(NCCN)cc(C(C)(C)C)c3)c2C)c1] |
| D11 | 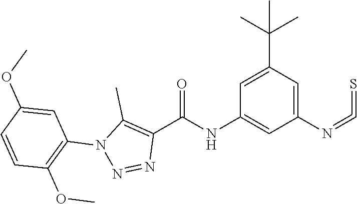 |
| D12 | 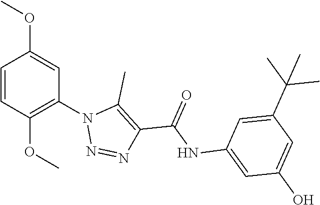 [SMILES: COc1ccc(OC)c(-n2nnc(C(=O)Nc3cc(O)cc(C(C)(C)C)c3)c2C)c1] |
| D13 | 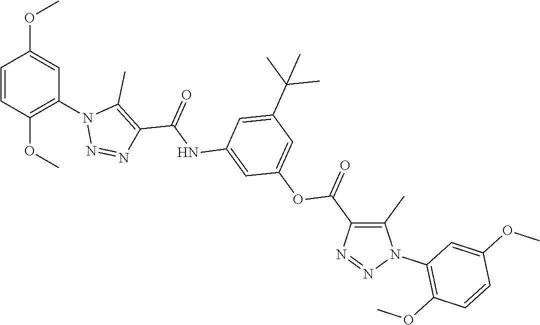 [SMILES: COc1ccc(OC)c(-n2nnc(C(=O)Nc3cc(OC(=O)c4nnn(-c5cc(OC)ccc5OC)c4C)cc(C(C)(C)C)c3)c2C)c1] |
| D14 | 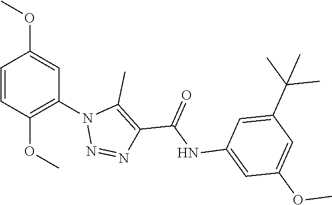 [SMILES: COc1cc(NC(=O)c2nnn(-c3cc(OC)ccc3OC)c2C)cc(C(C)(C)C)c1] |
| D15 | 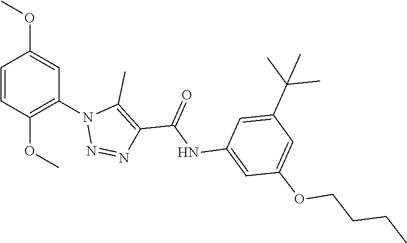 [SMILES: CCCCOc1cc(NC(=O)c2nnn(-c3cc(OC)ccc3OC)c2C)cc(C(C)(C)C)c1] |
| D16 | 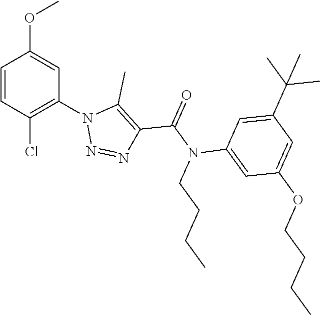 [SMILES: CCCCOc1cc(N(CCCC)C(=O)c2nnn(-c3cc(OC)ccc3Cl)c2C)cc(C(C)(C)C)c1] |
| D17 | 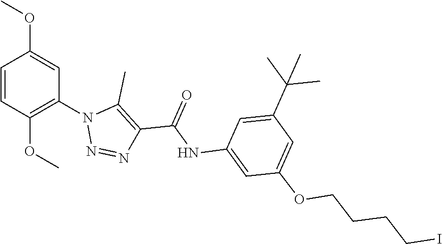 [SMILES: COc1ccc(OC)c(-n2nnc(C(=O)Nc3cc(OCCCCI)cc(C(C)(C)C)c3)c2C)c1] |
| D18 | 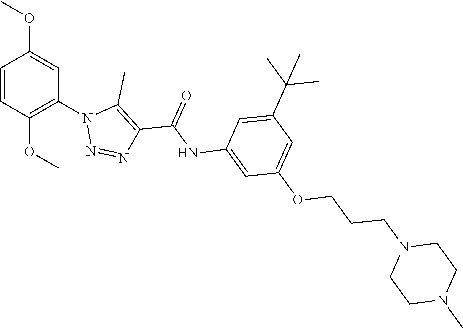 [SMILES: COc1ccc(OC)c(-n2nnc(C(=O)Nc3cc(OCCCN4CCN(C)CC4)cc(C(C)(C)C)c3)c2C)c1] |
| D19 | 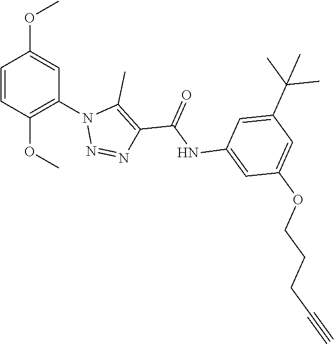 [SMILES: C#CCCCOc1cc(NC(=O)c2nnn(-c3cc(OC)ccc3OC)c2C)cc(C(C)(C)C)c1] |
| D20 | 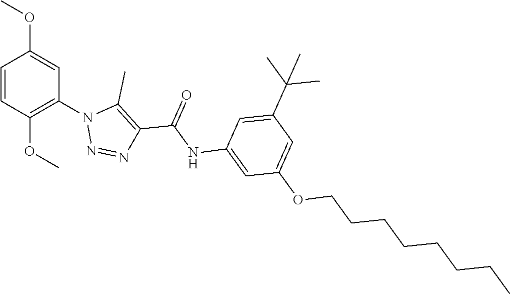 [SMILES: CCCCCCCCOc1cc(NC(=O)c2nnn(-c3cc(OC)ccc3OC)c2C)cc(C(C)(C)C)c1] |
| D21 | 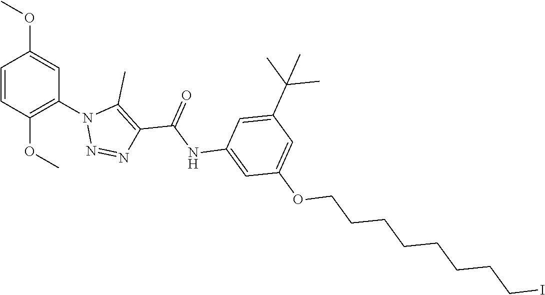 [SMILES: COc1ccc(OC)c(-n2nnc(C(=O)Nc3cc(OCCCCCCCCI)cc(C(C)(C)C)c3)c2C)c1] |
| D22 | 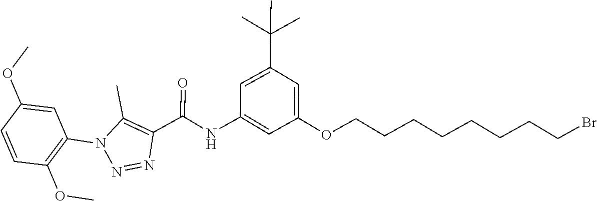 [SMILES: COc1ccc(OC)c(-n2nnc(C(=O)Nc3cc(OCCCCCCCCBr)cc(C(C)(C)C)c3)c2C)c1] |
| D23 | 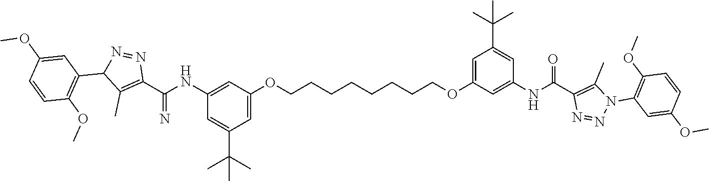 |
| D24 | 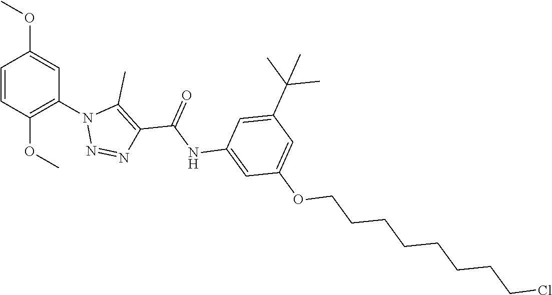 [SMILES: COc1ccc(OC)c(-n2nnc(C(=O)Nc3cc(OCCCCCCCCCl)cc(C(C)(C)C)c3)c2C)c1] |
| D25 | 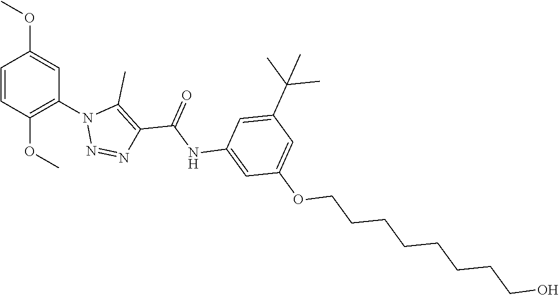 [SMILES: COc1ccc(OC)c(-n2nnc(C(=O)Nc3cc(OCCCCCCCCO)cc(C(C)(C)C)c3)c2C)c1] |
| D26 | 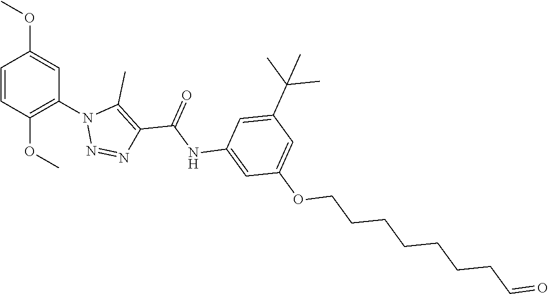 [SMILES: COc1ccc(OC)c(-n2nnc(C(=O)Nc3cc(OCCCCCCCC=O)cc(C(C)(C)C)c3)c2C)c1] |
| D27 | 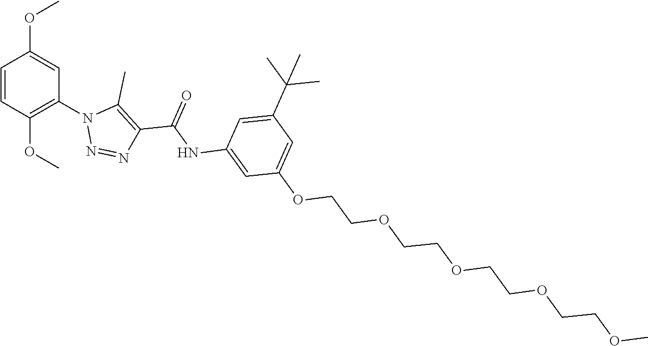 [SMILES: COCCOCCOCCOCCOc1cc(NC(=O)c2nnn(-c3cc(OC)ccc3OC)c2C)cc(C(C)(C)C)c1] |
| D28 | 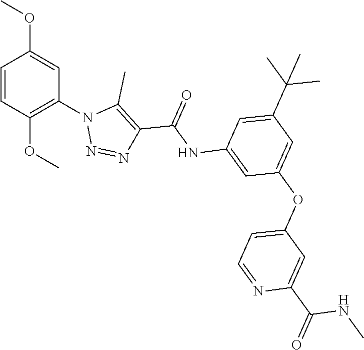 [SMILES: CNC(=O)c1cc(Oc2cc(NC(=O)c3nnn(-c4cc(OC)ccc4OC)c3C)cc(C(C)(C)C)c2)ccn1] |
| D29 | 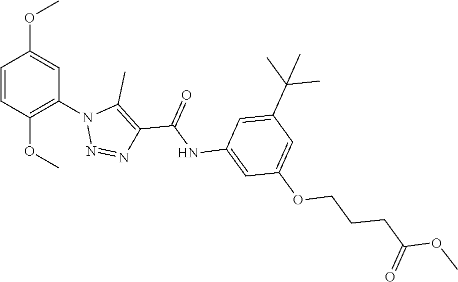 [SMILES: COC(=O)CCCOc1cc(NC(=O)c2nnn(-c3cc(OC)ccc3OC)c2C)cc(C(C)(C)C)c1] |
| D30 | 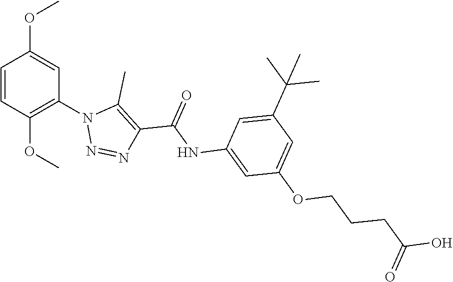 [SMILES: COc1ccc(OC)c(-n2nnc(C(=O)Nc3cc(OCCCC(=O)O)cc(C(C)(C)C)c3)c2C)c1] |
| D31 | 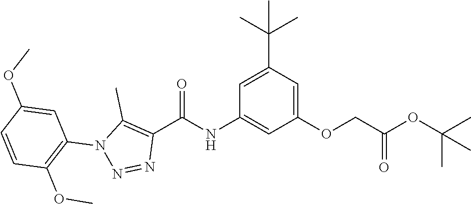 [SMILES: COc1ccc(OC)c(-n2nnc(C(=O)Nc3cc(OCC(=O)OC(C)(C)C)cc(C(C)(C)C)c3)c2C)c1] |
| D32 | 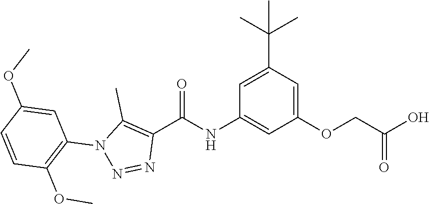 [SMILES: COc1ccc(OC)c(-n2nnc(C(=O)Nc3cc(OCC(=O)O)cc(C(C)(C)C)c3)c2C)c1] |
| D33 | 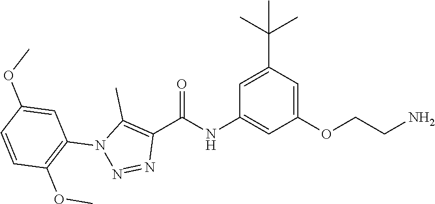 [SMILES: COc1ccc(OC)c(-n2nnc(C(=O)Nc3cc(OCCN)cc(C(C)(C)C)c3)c2C)c1] |
| D34 | 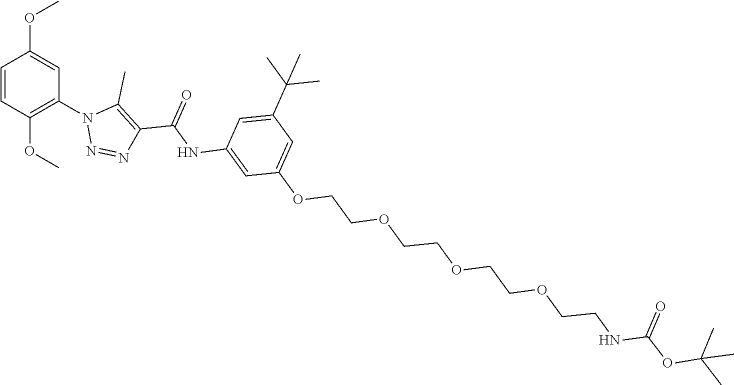 [SMILES: COc1ccc(OC)c(-n2nnc(C(=O)Nc3cc(OCCOCCOCCOCCNC(=O)OC(C)(C)C)cc(C(C)(C)C)c3)c2C)c1] |
| D35 | 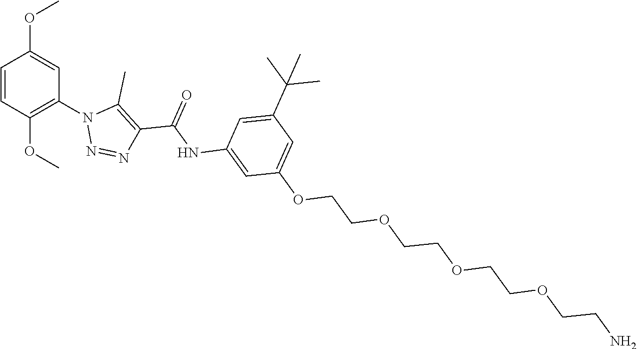 [SMILES: COc1ccc(OC)c(-n2nnc(C(=O)Nc3cc(OCCOCCOCCOCCN)cc(C(C)(C)C)c3)c2C)c1] |
| D36 | 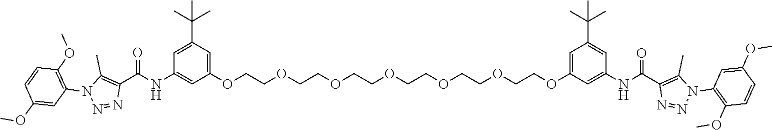 |
| D37 | 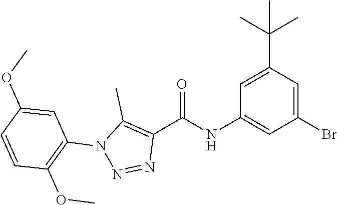 |
| D38 | 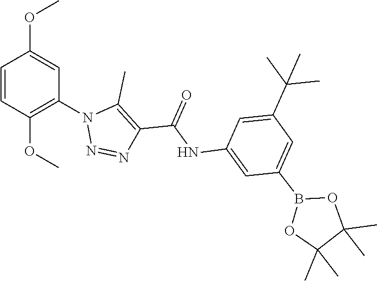 [SMILES: COc1ccc(OC)c(-n2nnc(C(=O)Nc3cc(B4OC(C)(C)C(C)(C)O4)cc(C(C)(C)C)c3)c2C)c1] |
| D39 | 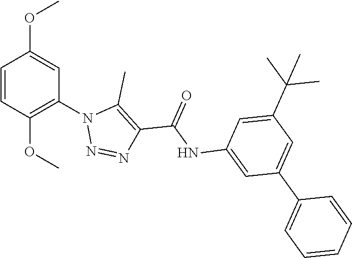 [SMILES: COc1ccc(OC)c(-n2nnc(C(=O)Nc3cc(-c4ccccc4)cc(C(C)(C)C)c3)c2C)c1] |
| D40 | 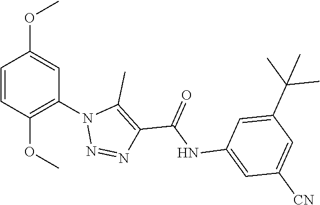 [SMILES: COc1ccc(OC)c(-n2nnc(C(=O)Nc3cc(C#N)cc(C(C)(C)C)c3)c2C)c1] |
| D41 | 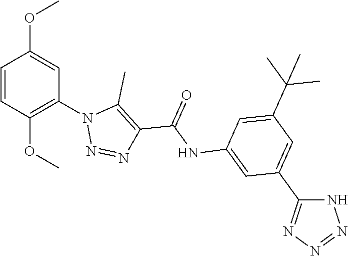 [SMILES: COc1ccc(OC)c(-n2nnc(C(=O)Nc3cc(-c4nnn[nH]4)cc(C(C)(C)C)c3)c2C)c1] |
| E1 | 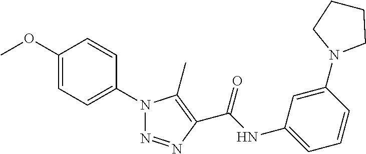 [SMILES: COc1ccc(-n2nnc(C(=O)Nc3cccc(N4CCCC4)c3)c2C)cc1] |
| E2 | 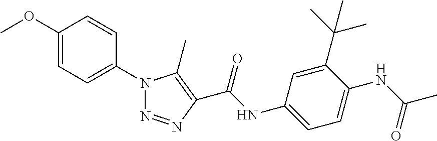 [SMILES: COc1ccc(-n2nnc(C(=O)Nc3ccc(NC(C)=O)c(C(C)(C)C)c3)c2C)cc1] |
| E3 | 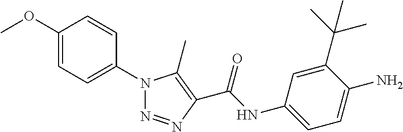 [SMILES: COc1ccc(-n2nnc(C(=O)Nc3ccc(N)c(C(C)(C)C)c3)c2C)cc1] |
| E4 | 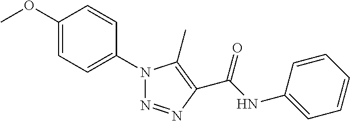 [SMILES: COc1ccc(-n2nnc(C(=O)Nc3ccccc3)c2C)cc1] |
| E5 | 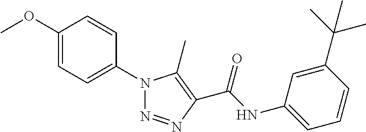 [SMILES: COc1ccc(-n2nnc(C(=O)Nc3cccc(C(C)(C)C)c3)c2C)cc1] |
| E6 | 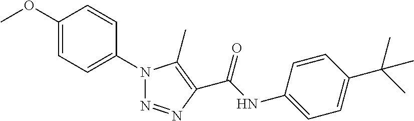 [SMILES: COc1ccc(-n2nnc(C(=O)Nc3ccc(C(C)(C)C)cc3)c2C)cc1] |
| E7 | 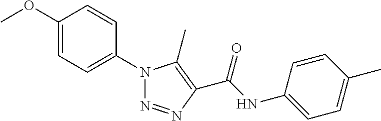 |
| E8 | 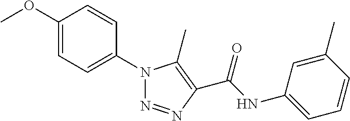 [SMILES: COc1ccc(-n2nnc(C(=O)Nc3cccc(C)c3)c2C)cc1] |
| E9 | 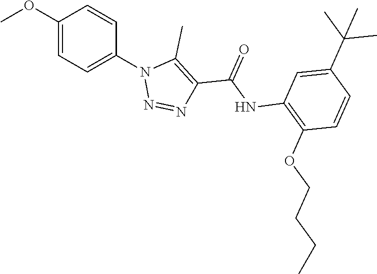 [SMILES: CCCCOc1ccc(C(C)(C)C)cc1NC(=O)c1nnn(-c2ccc(OC)cc2)c1C] |
| E10 | 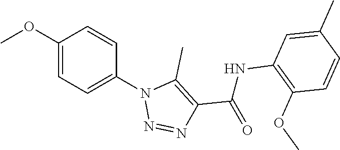 [SMILES: COc1ccc(-n2nnc(C(=O)Nc3cc(C)ccc3OC)c2C)cc1] |
| F1 | 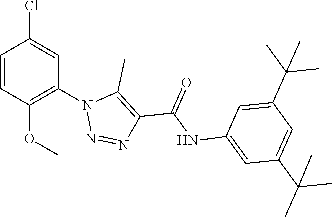 [SMILES: COc1ccc(Cl)cc1-n1nnc(C(=O)Nc2cc(C(C)(C)C)cc(C(C)(C)C)c2)c1C] |
| F2 | 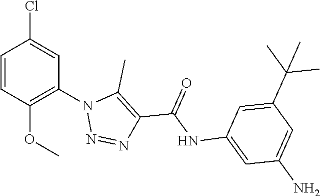 [SMILES: COc1ccc(Cl)cc1-n1nnc(C(=O)Nc2cc(N)cc(C(C)(C)C)c2)c1C] |
| F3 | 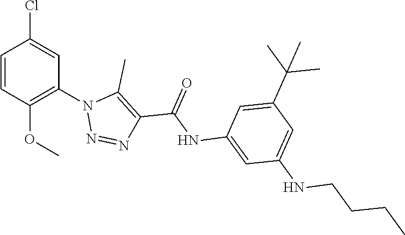 [SMILES: CCCCNc1cc(NC(=O)c2nnn(-c3cc(Cl)ccc3OC)c2C)cc(C(C)(C)C)c1] |
| F4 | 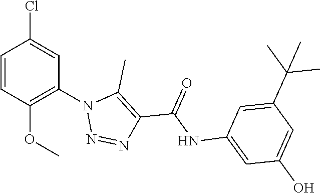 [SMILES: COc1ccc(Cl)cc1-n1nnc(C(=O)Nc2cc(O)cc(C(C)(C)C)c2)c1C] |
| F5 | 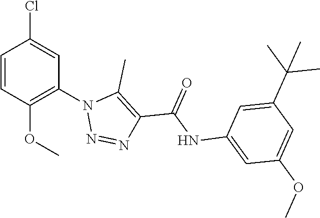 [SMILES: COc1cc(NC(=O)c2nnn(-c3cc(Cl)ccc3OC)c2C)cc(C(C)(C)C)c1] |
| F6 | 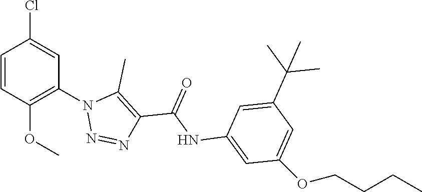 [SMILES: CCCCOc1cc(NC(=O)c2nnn(-c3cc(Cl)ccc3OC)c2C)cc(C(C)(C)C)c1] |
| F7 | 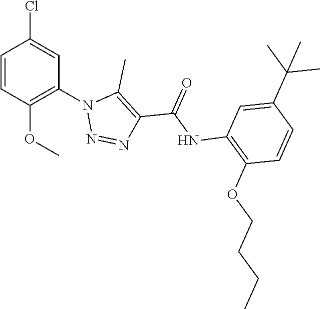 [SMILES: CCCCOc1ccc(C(C)(C)C)cc1NC(=O)c1nnn(-c2cc(Cl)ccc2OC)c1C] |
| F8 | 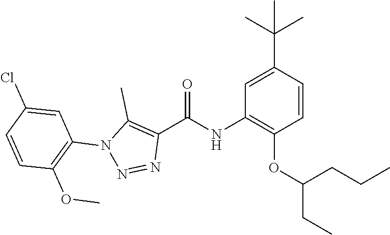 |
| F9 | 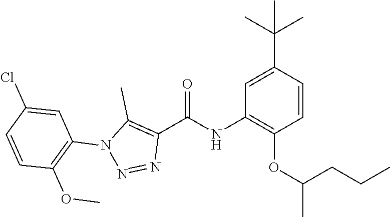 |
| F10 | 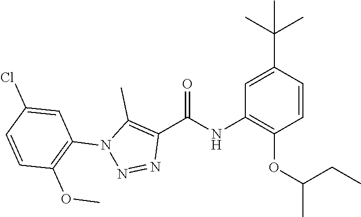 |
| G1 | 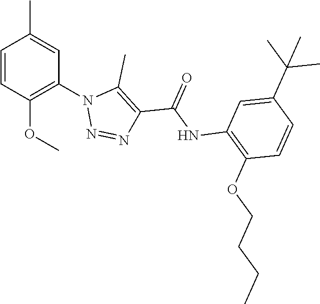 [SMILES: CCCCOc1ccc(C(C)(C)C)cc1NC(=O)c1nnn(-c2cc(C)ccc2OC)c1C] |
| G2 | 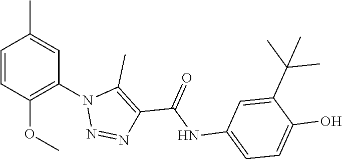 [SMILES: COc1ccc(C)cc1-n1nnc(C(=O)Nc2ccc(O)c(C(C)(C)C)c2)c1C] |
| G3 | 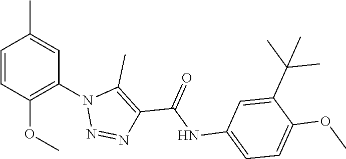 [SMILES: COc1ccc(C)cc1-n1nnc(C(=O)Nc2ccc(OC)c(C(C)(C)C)c2)c1C] |
| G4 | 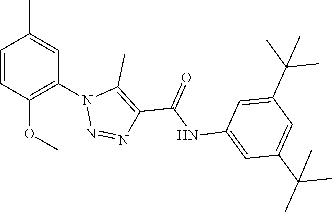 [SMILES: COc1ccc(C)cc1-n1nnc(C(=O)Nc2cc(C(C)(C)C)cc(C(C)(C)C)c2)c1C] |
| G5 | 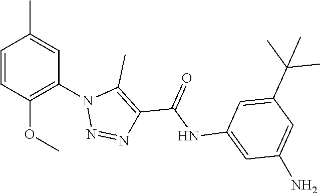 [SMILES: COc1ccc(C)cc1-n1nnc(C(=O)Nc2cc(N)cc(C(C)(C)C)c2)c1C] |
| G6 | 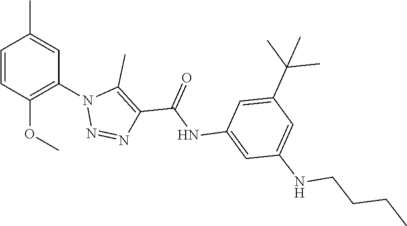 [SMILES: CCCCNc1cc(NC(=O)c2nnn(-c3cc(C)ccc3OC)c2C)cc(C(C)(C)C)c1] |
| G7 | 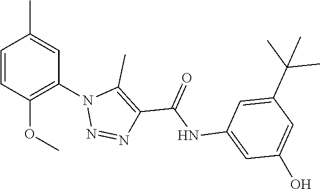 [SMILES: COc1ccc(C)cc1-n1nnc(C(=O)Nc2cc(O)cc(C(C)(C)C)c2)c1C] |
| G8 | 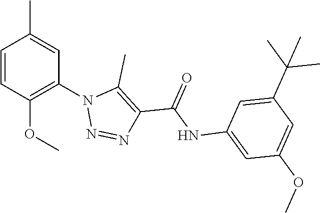 [SMILES: COc1cc(NC(=O)c2nnn(-c3cc(C)ccc3OC)c2C)cc(C(C)(C)C)c1] |
| G9 | 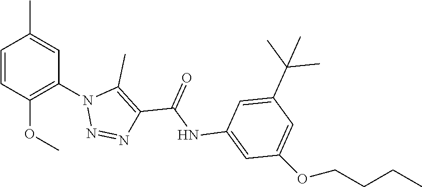 [SMILES: CCCCOc1cc(NC(=O)c2nnn(-c3cc(C)ccc3OC)c2C)cc(C(C)(C)C)c1] |
| G10 | 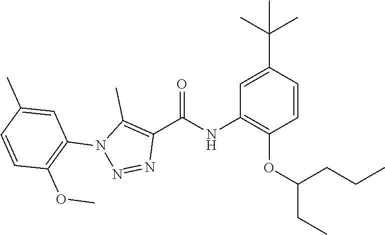 |
| G11 | 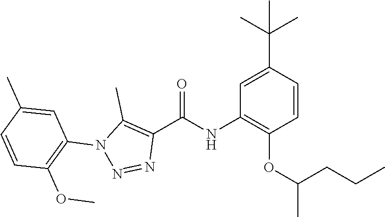 |
| G12 | 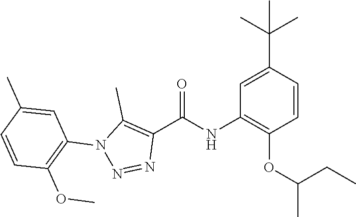 |
| H1 | 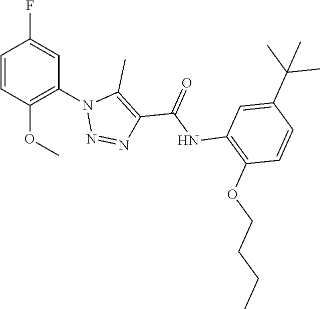 [SMILES: CCCCOc1ccc(C(C)(C)C)cc1NC(=O)c1nnn(-c2cc(F)ccc2OC)c1C] |
| H2 | 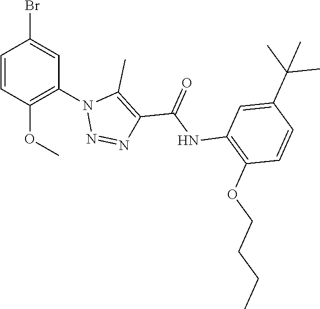 [SMILES: CCCCOc1ccc(C(C)(C)C)cc1NC(=O)c1nnn(-c2cc(Br)ccc2OC)c1C] |
| H3 | 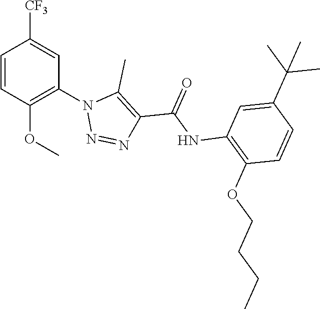 [SMILES: CCCCOc1ccc(C(C)(C)C)cc1NC(=O)c1nnn(-c2cc(C(F)(F)F)ccc2OC)c1C] |
| H4 | 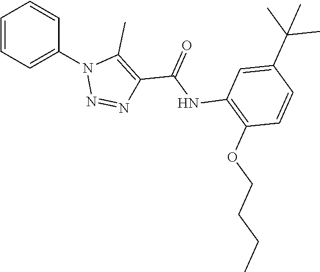 [SMILES: CCCCOc1ccc(C(C)(C)C)cc1NC(=O)c1nnn(-c2ccccc2)c1C] |
| H5 | 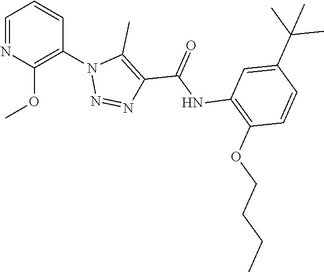 [SMILES: CCCCOc1ccc(C(C)(C)C)cc1NC(=O)c1nnn(-c2cccnc2OC)c1C] |
| H6 | 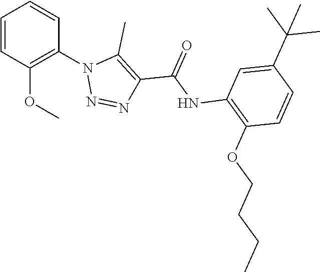 [SMILES: CCCCOc1ccc(C(C)(C)C)cc1NC(=O)c1nnn(-c2ccccc2OC)c1C] |
| H7 | 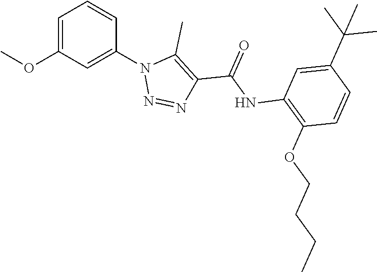 [SMILES: CCCCOc1ccc(C(C)(C)C)cc1NC(=O)c1nnn(-c2cccc(OC)c2)c1C] |
| H8 | 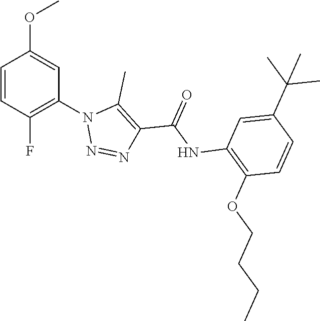 [SMILES: CCCCOc1ccc(C(C)(C)C)cc1NC(=O)c1nnn(-c2cc(OC)ccc2F)c1C] |
| H9 | 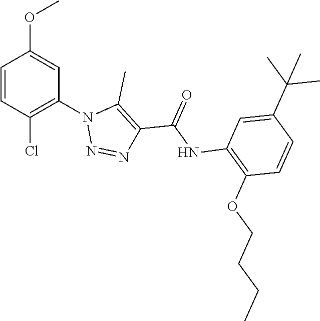 [SMILES: CCCCOc1ccc(C(C)(C)C)cc1NC(=O)c1nnn(-c2cc(OC)ccc2Cl)c1C] |
| H10 | 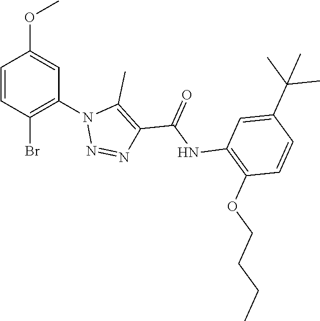 [SMILES: CCCCOc1ccc(C(C)(C)C)cc1NC(=O)c1nnn(-c2cc(OC)ccc2Br)c1C] |
| H11 | 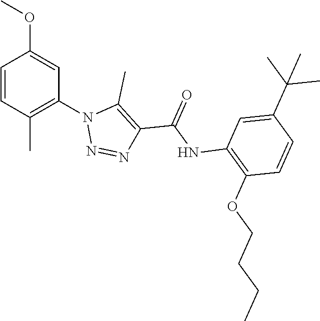 [SMILES: CCCCOc1ccc(C(C)(C)C)cc1NC(=O)c1nnn(-c2cc(OC)ccc2C)c1C] |
| H12 | 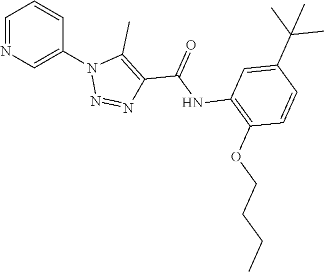 [SMILES: CCCCOc1ccc(C(C)(C)C)cc1NC(=O)c1nnn(-c2cccnc2)c1C] |
| H13 | 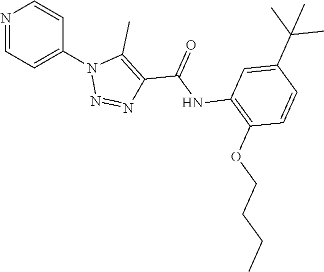 [SMILES: CCCCOc1ccc(C(C)(C)C)cc1NC(=O)c1nnn(-c2ccncc2)c1C] |
| H14 | 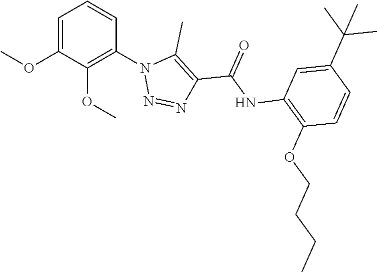 [SMILES: CCCCOc1ccc(C(C)(C)C)cc1NC(=O)c1nnn(-c2cccc(OC)c2OC)c1C] |
| H15 | 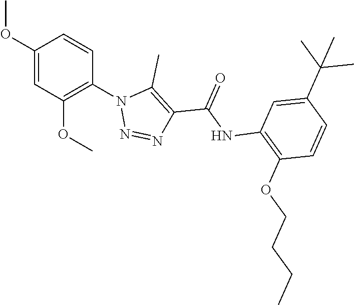 [SMILES: CCCCOc1ccc(C(C)(C)C)cc1NC(=O)c1nnn(-c2ccc(OC)cc2OC)c1C] |
| H16 | 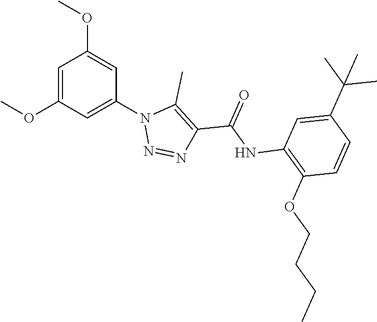 [SMILES: CCCCOc1ccc(C(C)(C)C)cc1NC(=O)c1nnn(-c2cc(OC)cc(OC)c2)c1C] |
| H17 | 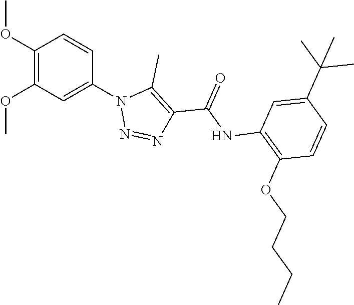 [SMILES: CCCCOc1ccc(C(C)(C)C)cc1NC(=O)c1nnn(-c2ccc(OC)c(OC)c2)c1C] |
| H18 | 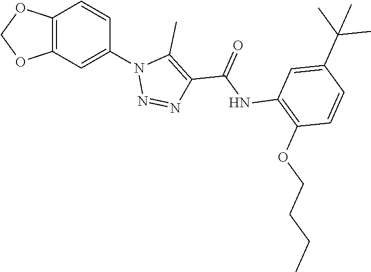 [SMILES: CCCCOc1ccc(C(C)(C)C)cc1NC(=O)c1nnn(-c2ccc3c(c2)OCO3)c1C] |
| H19 | 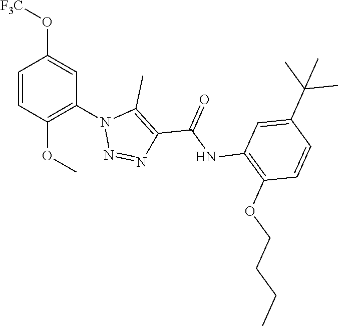 [SMILES: CCCCOc1ccc(C(C)(C)C)cc1NC(=O)c1nnn(-c2cc(OC(F)(F)F)ccc2OC)c1C] |
| H20 | 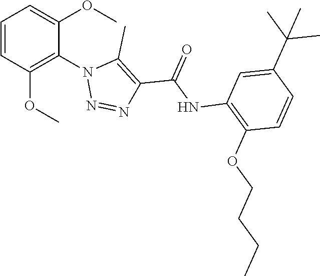 [SMILES: CCCCOc1ccc(C(C)(C)C)cc1NC(=O)c1nnn(-c2c(OC)cccc2OC)c1C] |
| I1 | 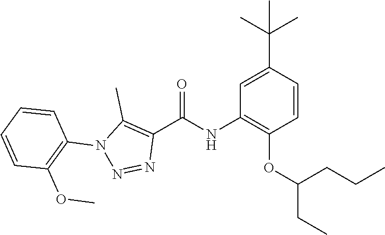 |
| I2 | 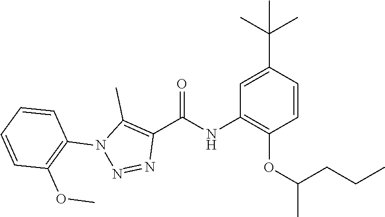 |
| I3 | 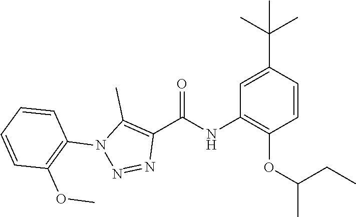 |
| J1 | 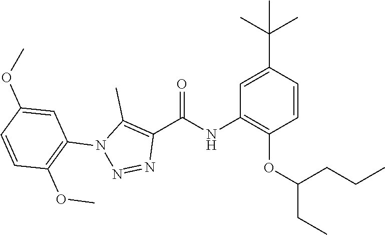 |
| J2 | 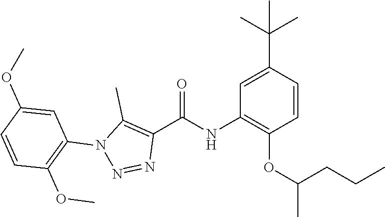 |
| J3 | 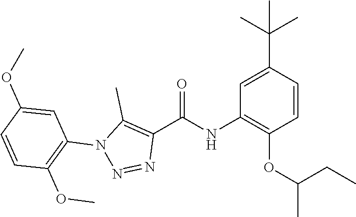 |
| K1 | 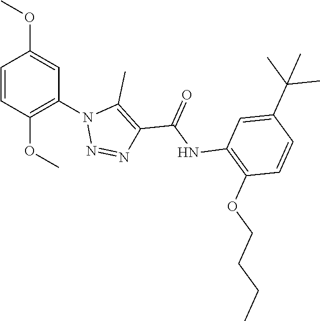 [SMILES: CCCCOc1ccc(C(C)(C)C)cc1NC(=O)c1nnn(-c2cc(OC)ccc2OC)c1C] |
| K2 | 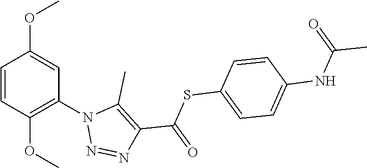 [SMILES: COc1ccc(OC)c(-n2nnc(C(=O)Sc3ccc(NC(C)=O)cc3)c2C)c1] |
| K3 | 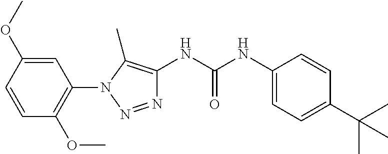 [SMILES: COc1ccc(OC)c(-n2nnc(NC(=O)Nc3ccc(C(C)(C)C)cc3)c2C)c1] |
| K4 | 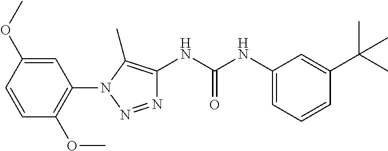 [SMILES: COc1ccc(OC)c(-n2nnc(NC(=O)Nc3cccc(C(C)(C)C)c3)c2C)c1] |
| K5 | 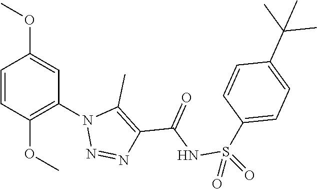 [SMILES: COc1ccc(OC)c(-n2nnc(C(=O)NS(=O)(=O)c3ccc(C(C)(C)C)cc3)c2C)c1] |
| K6 | 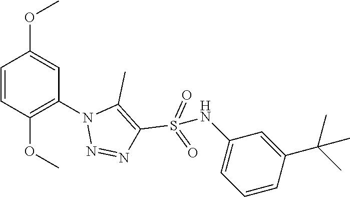 [SMILES: COc1ccc(OC)c(-n2nnc(S(=O)(=O)Nc3cccc(C(C)(C)C)c3)c2C)c1] |
| K7 | 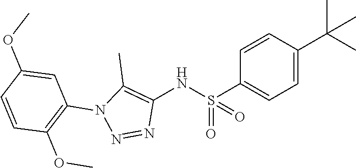 [SMILES: COc1ccc(OC)c(-n2nnc(NS(=O)(=O)c3ccc(C(C)(C)C)cc3)c2C)c1] |
| K8 | 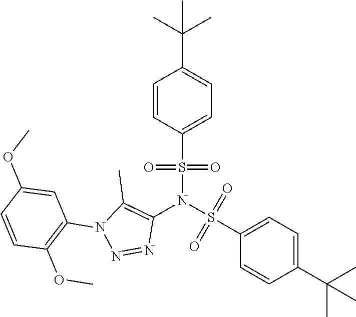 [SMILES: COc1ccc(OC)c(-n2nnc(N(S(=O)(=O)c3ccc(C(C)(C)C)cc3)S(=O)(=O)c3ccc(C(C)(C)C)cc3)c2C)c1] |
| L1 | 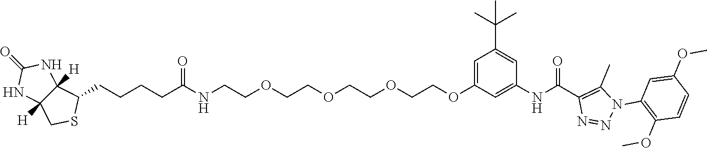 [SMILES: COc1ccc(OC)c(-n2nnc(C(=O)Nc3cc(OCCOCCOCCOCCNC(=O)CCCC[C@@H]4SC[C@@H]5NC(=O)N[C@@H]54)cc(C(C)(C)C)c3)c2C)c1] |
| L2 | 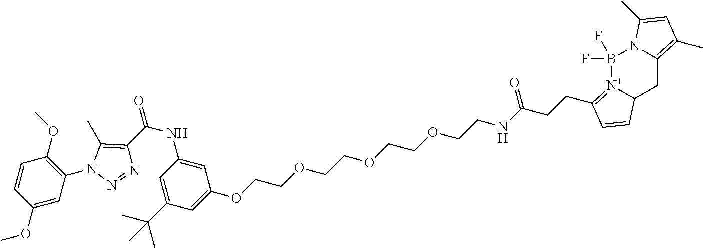 |
| M1 | 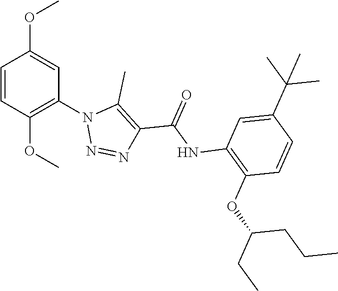 [SMILES: CCC[C@H](CC)Oc1ccc(C(C)(C)C)cc1NC(=O)c1nnn(-c2cc(OC)ccc2OC)c1C] |
| M2 | 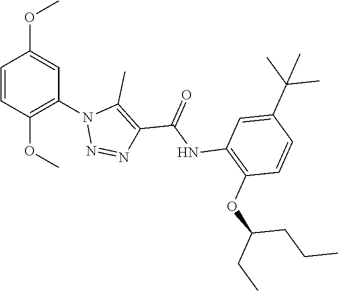 [SMILES: CCC[C@@H](CC)Oc1ccc(C(C)(C)C)cc1NC(=O)c1nnn(-c2cc(OC)ccc2OC)c1C] |
| M3 | 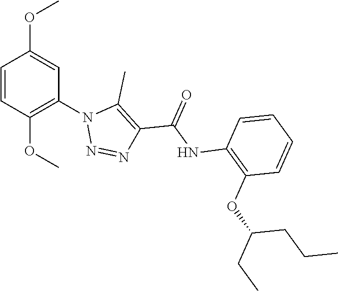 [SMILES: CCC[C@H](CC)Oc1ccccc1NC(=O)c1nnn(-c2cc(OC)ccc2OC)c1C] |
| M4 | 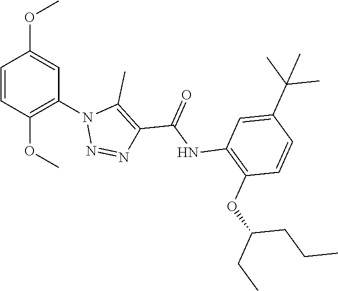 [SMILES: CCC[C@H](CC)Oc1ccc(C(C)(C)C)cc1NC(=O)c1nnn(-c2cc(OC)ccc2OC)c1C] |
| M5 | 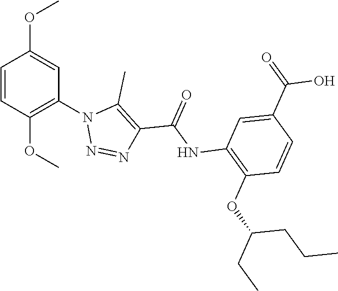 [SMILES: CCC[C@H](CC)Oc1ccc(C(=O)O)cc1NC(=O)c1nnn(-c2cc(OC)ccc2OC)c1C] |
| M6 | 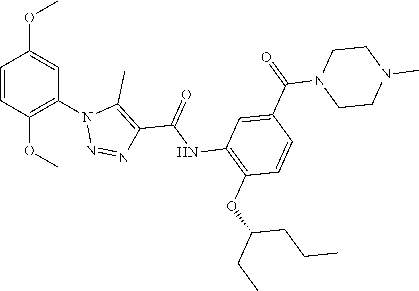 [SMILES: CCC[C@H](CC)Oc1ccc(C(=O)N2CCN(C)CC2)cc1NC(=O)c1nnn(-c2cc(OC)ccc2OC)c1C] |
| M7 | 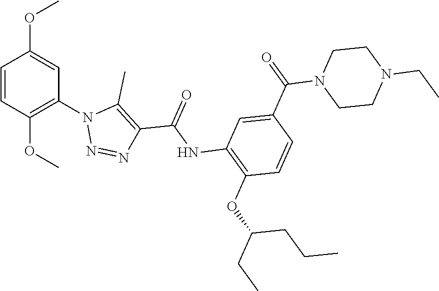 [SMILES: CCC[C@H](CC)Oc1ccc(C(=O)N2CCN(CC)CC2)cc1NC(=O)c1nnn(-c2cc(OC)ccc2OC)c1C] |
| M8 | 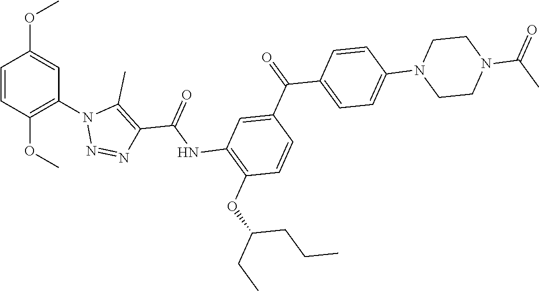 [SMILES: CCC[C@H](CC)Oc1ccc(C(=O)c2ccc(N3CCN(C(C)=O)CC3)cc2)cc1NC(=O)c1nnn(-c2cc(OC)ccc2OC)c1C] |
| M9 | 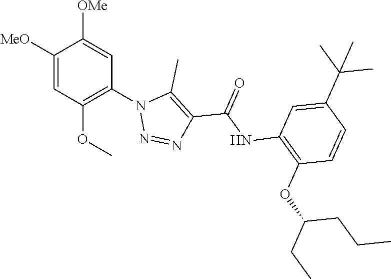 [SMILES: CCC[C@H](CC)Oc1ccc(C(C)(C)C)cc1NC(=O)c1nnn(-c2cc(OC)c(OC)cc2OC)c1C] |
| M10 | 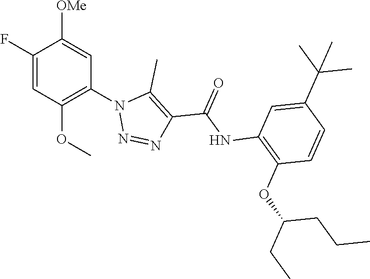 [SMILES: CCC[C@H](CC)Oc1ccc(C(C)(C)C)cc1NC(=O)c1nnn(-c2cc(OC)c(F)cc2OC)c1C] |
| M11 | 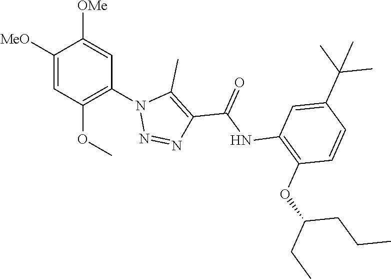 [SMILES: CCC[C@H](CC)Oc1ccc(C(C)(C)C)cc1NC(=O)c1nnn(-c2cc(OC)c(OC)cc2OC)c1C] |
| M12 | 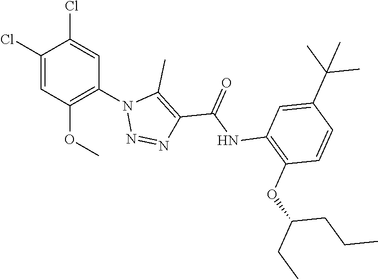 [SMILES: CCC[C@H](CC)Oc1ccc(C(C)(C)C)cc1NC(=O)c1nnn(-c2cc(Cl)c(Cl)cc2OC)c1C] |

The activity of the exemplary compounds is shown in Table 2 below.

TABLE 2

| | | | Inverse | |
|---|---|---|---|---|
| | Binding^{a} | Agonist^{b} | agonist^{c} | Antagonist^{d} |
| | (IC₅₀/μM) | (EC₅₀/μM) | (IC₅₀/μM) | (IC₅₀/μM) |
| | (% inhibition | (% activation | (% inhibition | (% inhibition |
| Compound | at 10 μM) | at 10 μM) | at 10 μM) | at 10 μM) |
| T0901317 | 0.026 ± 0.0034 | NT^{e} | NT | NT |
| | (100 ± 1)% | | | |
| Rifampicin | NT | 1.17 ± 0.095 | NA | NT |
| | | (100 ± 2)% | | |
| SPA70 | 0.21 ± 0.037 | NA^{f} | 0.022 ± 0.0035 | 0.23 ± 0.024 |
| | (87 ± 3)% | | (100 ± 2)% | (100 ± 1)% |
| A1 | NA | 24.51 ± 4.76 | NA | NA |
| | | (33 ± 2)% | | |
| A2 | 14.0 ± 4.79 | 2.44 ± 1.02 | NA | NA |
| | (22 ± 7)% | (32 ± 7)% | | |
| A3 | 10.9 ± 0.4 | 3.51 ± 0.86 | NA | NA |
| | (52 ± 5)% | (30 ± 3)% | | |
| A4 | 1.18 ± 0.38 | NA | NA | 33.92 ± 18.3 |
| | (76 ± 7)% | | | (58 ± 9)% |
| A5 | 0.65 ± 0.32 | NA | 0.48 ± 0.1 | 4.07 ± 1.47 |
| | (78 ± 2)% | | (102 ± 11)% | (92 ± 8)% |
| A6 | 2.97 ± 0.07 | 0.74 ± 0.11 | NA | 29.76 ± 2.10 |
| | (62 ± 4)% | (35 ± 2)% | | (37 ± 7)% |
| A7 | NA | NA | 10.31 ± 3.25 | NA |
| | | | (80 ± 12)% | |
| A8 | 16.1 ± 0.54 | NA | NA | NA |
| | (49 ± 1)% | | | |
| A9 | 1.01 ± 0.56 | NA | 3.03 ± 0.79 | 4.79 ± 0.41 |
| | (74 ± 8)% | | (123 ± 18)% | (82 ± 6)% |
| A10 | 10.5 ± 2.1 | NA | NA | NA |
| | (49 ± 4)% | | | |
| A11 | 1.41 ± 0.69 | NA | NA | NA |
| | (24 ± 6)% | | | |
| A12 | 9.37 ± 3.52 | NA | 25.83 ± 6.27 | NA |
| | (50 ± 4)% | | (36 ± 4)% | |
| A13 | 2.67 ± 0.48 | NA | 8.75 ± 1.26 | 20.77 ± 3.49 |
| | (64 ± 4)% | | (64 ± 8)% | (56 ± 4)% |
| A14 | NA | NA | NA | NA |
| A15 | NA | NA | NA | NA |
| A16 | NA | NA | NA | NA |
| A17 | NA | NA | NA | NA |
| A18 | 12.55 ± 3.09 | NA | NA | NA |
| | (36 ± 3)% | | | |
| A19 | NA | 10.17 ± 2.46 | NA | NA |
| | | (34 ± 6)% | | |
| A20 | NA | NA | NA | NA |
| A21 | NA | NA | NA | NA |
| A22 | NA | NA | 10.61 ± 8.63 | NA |
| | | | (129 ± 18)% | |
| A23 | NA | NA | NA | 13.4 ± 0.12 |
| | | | | (23 ± 3)% |
| A24 | 0.21 ± 0.06 | NA | 0.049 ± 0.0028 | 0.15 ± 0.026 |
| | (65 ± 0.5)% | | (108 ± 5)% | (107 ± 3)% |
| A25 | 1.36 ± 0.23 | NA | 20.6 ± 0.42 | 12 ± 2 |
| | (73 ± 0.4)% | | (53 ± 9)% | (70 ± 5)% |
| B1 | 1.12 ± 0.29 | NA | NA | 1.79 ± 0.32 |
| | (64 ± 7)% | | | (80 ± 1)% |
| B2 | 2.33 ± 0.43 | NA | NA | NA |
| | (84 ± 3)% | | | |
| B3 | NA | 0.59 ± 0.093 | NA | NA |
| | | (55 ± 6)% | | |
| B4 | 0.167 ± 0.042 | NA | 0.077 ± 0.0021 | 0.46 ± 0.071 |
| | (73 ± 3)% | | (118 ± 2)% | (105 ± 4)% |
| B5 | 0.027 ± 0.0062 | NA | 0.055 ± 0.01 | 0.28 ± 0.027 |
| | (79 ± 2)% | | (114 ± 13)% | (101 ± 4)% |
| B6 | 0.022 ± 0.012 | NA | 0.024 ± 0.006 | 0.24 ± 0.082 |
| | (90 ± 3)% | | (89 ± 9)% | (101 ± 2)% |
| B7 | 0.039 ± 0.011 | NA | 0.043 ± 0.0028 | 0.21 ± 0.012 |
| | (86 ± 1)% | | (109 ± 5)% | (103 ± 2)% |
| B8 | 0.027 ± 0.0066 | NA | 0.044 ± 0.0092 | 0.24 ± 0.025 |
| | (73 ± 3)% | | (116 ± 6)% | (101 ± 5)% |
| B9 | 0.58 ± 0.15 | NA | 0.44 ± 0.14 | 2.23 ± 0.028 |
| | (53 ± 0.4)% | | (83 ± 12)% | (72 ± 6)% |
| B10 | 2.34 ± 0.57 | NA | 0.43 ± 0.14 | 1.92 ± 0.23 |
| | (62 ± 4)% | | (95 ± 1)% | (102 ± 4)% |
| B11 | 1.24 ± 0.34 | NA | 5.11 ± 1.86 | 3.35 ± 0.014 |
| | (78 ± 2)% | | (157 ± 18)% | (93 ± 4)% |
| B12 | 0.021 ± 0.005 | NA | 0.0074 ± 0.00061 | 0.049 ± 0.01 |
| | (88 ± 2)% | | (119 ± 2)% | (107 ± 3)% |
| B13 | 1.3 ± 0.23 | NA | | 1.25 ± 0.14 |
| | (48 ± 5)% | | | (49 ± 6)% |
| B14 | 0.28 ± 0.089 | NA | 1.83 ± 0.12 | 1.67 ± 0.041 |
| | (87 ± 1)% | | (113 ± 11)% | (105 ± 3)% |
| B15 | 0.54 ± 0.073 | NA | 1.35 ± 0.44 | 4.61 ± 1.07 |
| | (68 ± 5)% | | (81 ± 7)% | (91 ± 4)% |
| B16 | NA | NA | 10.09 ± 2.70 | 7.03 ± 0.91 |
| | | | (127 ± 7)% | (109 ± 4)% |
| B17 | 30.95 ± 4.83 | NA | 8.86 ± 1.07 | 7.19 ± 0.82 |
| | (47 ± 3)% | | (137 ± 12)% | (112 ± 3)% |
| B18 | 6.46 ± 0.61 | NA | 4.66 ± 2.87 | 12.23 ± 0.77 |
| | (49 ± 4)% | | (86 ± 3)% | (73 ± 3)% |
| B19 | 0.50 ± 0.12 | NA | 0.43 ± 0.14 | 2.48 ± 0.082 |
| | (68 ± 1)% | | (95 ± 1)% | (86 ± 3)% |
| B20 | NA | NA | NA | NA |
| B21 | 0.055 ± 0.0014 | NA | 0.038 ± 0.022 | 0.31 ± 0.15 |
| | ( )% | | (24 ± 6)% | (91 ± 1)% |
| B22 | NA | NA | 3.08 ± 0.55 | 6.09 ± 0.85 |
| | | | (168 ± 16)% | (114 ± 6)% |
| B23 | NA | NA | 16.66 ± 3.34 | NA |
| | | | (45 ± 8)% | |
| B24 | 0.5 ± 0.2 | 0.27 ± 0.023 | NA | NA |
| | (74 ± 6)% | (77 ± 1)% | | |
| B25 | 0.36 ± 0.072 | 10.7 ± 1.12 | NA | NA |
| | (90 ± 3)% | (81 ± 9)% | | |
| B26 | 0.0044 ± 0.00073 | NA | 0.0037 ± 0.00078 | 0.027 ± 0.0023 |
| | (89 ± 1)% | | (111 ± 5)% | (106 ± 3)% |
| B27 | 0.0062 ± 0.0021 | NA | 0.0016 ± 0.00047 | 0.014 ± 0.0022 |
| | (90 ± 2)% | | (117 ± 6)% | (107 ± 3)% |
| B28 | 0.0119 ± 0.0041 | NA | 0.042 ± 0.0064 | 0.25 ± 0.023 |
| | (82 ± 3)% | | (94 ± 4)% | (98 ± 3)% |
| B29 | 0.0177 ± 0.0031 | NA | 0.026 ± 0.0069 | 0.18 ± 0.015 |
| | (85 ± 2)% | | (100 ± 4)% | (99 ± 1)% |
| B30 | 0.0074 ± 0.0001 | NA | 0.013 ± 0.0018 | 0.11 ± 0.015 |
| | (86 ± 4)% | | (67 ± 4)% | (94 ± 1)% |
| B31 | 0.0075 ± 0.0011 | NA | 0.0016 ± 0.00044 | 0.015 ± 0.0025 |
| | (91 ± 2)% | | (111 ± 8)% | (105 ± 2)% |
| B32 | 0.018 ± 0.0051 | NA | 0.021 ± 00049 | 0.26 ± 0.0049 |
| | (81 ± 3)% | | (89 ± 9)% | (96 ± 1)% |
| B33 | 0.018 ± 0.0011 | NA | 0.0049 ± 0.00062 | 0.039 ± 0.0043 |
| | (90 ± 5)% | | (103 ± 4)% | (106 ± 3)% |
| B34 | 0.019 ± 0.0005 | NA | 0.0023 ± 0.0008 | 0.012 ± 0.003 |
| | (92 ± 1)% | | (147 ± 18)% | (108 ± 4)% |
| B35 | 0.012 ± 0.0021 | NA | 0.0030 ± 0.0011 | 0.016 ± 0.0023 |
| | (87 ± 3)% | | (115 ± 1)% | (110 ± 8)% |
| B36 | 0.36 ± 0.03 | 0.15 ± 0.018 | NA | 11.69 ± 4.47 |
| | (75 ± 4)% | (63 ± 7)% | | (24 ± 6)% |
| B37 | NA | 0.90 ± 0.22 | NA | NA |
| | | (25 ± 7)% | | |
| B38 | 3.22 ± 2.29 | 0.99 ± 0.22 | NA | NA |
| | (60 ± 9)% | (60 ± 13)% | | |
| C1 | 1.81 ± 0.52 | 1.96 ± 0.19 | NA | NA |
| | (65 ± 7)% | (50 ± 14)% | | |
| C2 | 4.12 ± 1.40 | 0.28 ± 0.085 | NA | NA |
| | (86 ± 3)% | (33 ± 7)% | | |
| C3 | 0.47 ± 0.09 | 0.57 ± 0.044 | NA | 7.49 ± 0.93 |
| | (78 ± 1)% | (47 ± 4)% | | (44 ± 5)% |
| C4 | 0.73 ± 0.08 | NA | NA | 3.87 ± 0.38 |
| | (84 ± 2)% | | | (64 ± 4)% |
| C5 | 0.75 ± 2 | 0.3 ± 0.035 | NA | 14.12 ± 3.66 |
| | (92 ± 3)% | (28 ± 18)% | | (68 ± 4)% |
| C6 | 4.12 ± 0.48 | NA | 0.21 ± 0.077 | 0.86 ± 0.13 |
| | (69 ± 1)% | | (138 ± 24)% | (108 ± 5)% |
| C7 | 2.97 ± 0.30 | NA | 0.34 ± 0.12 | 1.71 ± 0.25 |
| | (66 ± 1)% | | (95 ± 26)% | (107 ± 6)% |
| C8 | NA | NA | 3.2 ± 0.34 | 7.32 ± 0.94 |
| | | | (73 ± 1)% | (60 ± 5)% |
| D1 | 3.01 ± 0.05 | NA | 2.26 ± 0.23 | 3.94 ± 0.23 |
| | (64 ± 2)% | | (87 ± 1)% | (93 ± 6)% |
| D2 | 0.33 ± 0.11 | NA | 0.12 ± 0.018 | 0.41 ± 0.13 |
| | (77 ± 6)% | | (126 ± 4)% | (109 ± 6)% |
| D3 | 0.27 ± 0.04 | NA | 0.11 ± 0.024 | 0.34 ± 0.036 |
| | (70 ± 2)% | | (120 ± 5)% | (110 ± 3)% |
| D4 | 0.45 ± 0.12 | NA | 0.039 ± 0.012 | 0.13 ± 0.027 |
| | (76 ± 1)% | | (110 ± 12)% | (109 ± 5)% |
| D5 | 0.50 ± 0.12 | NA | 0.15 ± 0.046 | 0.62 ± 0.28 |
| | (76 ± 2)% | | (89 ± 7)% | (82 ± 17)% |
| D6 | 1.26 ± 0.071 | NA | 12.72 ± 4.69 | 22.12 ± 4.86 |
| | (70 ± 5)% | | (48 ± 7)% | (25 ± 6)% |
| D7 | 0.57 ± 0.069 | NA | 0.025 ± 0.0036 | 0.33 ± 0.057 |
| | (77 ± 2)% | | (81 ± 5)% | (101 ± 3)% |
| D8 | 0.33 ± 0.045 | NA | 0.12 ± 0.00071 | 0.46 ± 0.06 |
| | (81 ± 4)% | | (127 ± 11)% | (104 ± 1)% |
| D9 | 3.53 ± 0.58 | NA | 1.7 ± 0.33 | 17.27 ± 1.39 |
| | (61 ± 1)% | | (107 ± 10)% | (65 ± 2)% |
| D10 | NA | NA | 7.09 ± 1.37 | 11.89 ± 2.27 |
| | | | (130 ± 9)% | (104 ± 4)% |
| D11 | 2.60 ± 0.51 | NA | 2.55 ± 0.64 | 30.2 ± 16.1 |
| | (47 ± 2)% | | (95 ± 3)% | (26 ± 2)% |
| D12 | 2.75 ± 0.77 | NA | 0.34 ± 0.043 | 2.04 ± 0.32 |
| | (82 ± 4)% | | (103 ± 10)% | (104 ± 3)% |
| D13 | NA | NA | 0.15 ± 0.0065 | 0.83 ± 0.11 |
| | | | (93 ± 9)% | (68 ± 2)% |
| D14 | 0.70 ± 0.15 | NA | 0.12 ± 0.025 | 0.50 ± 0.17 |
| | (84 ± 8)% | | (114 ± 3)% | (108 ± 5)% |
| D15 | 0.63 ± 0.10 | NA | 0.054 ± 0.0066 | 0.21 ± 0.086 |
| | (70 ± 2)% | | (113 ± 1)% | (107 ± 3)% |
| D16 | 0.069 ± 0.002 | NA | NA | 0.33 ± 0.068 |
| | (74 ± 4)% | | | (78 ± 9)% |
| D17 | 0.18 ± 0.043 | NA | 0.0088 ± 0.003 | 0.066 ± 0.007 |
| | (50 ± 6)% | | (120 ± 8)% | (112 ± 4)% |
| D18 | NA | NA | 3.73 ± 0.3 | 5.32 ± 0.053 |
| | | | (126 ± 18)% | (105 ± 6)% |
| D19 | 0.024 ± 0.0008 | NA | 0.032 ± 0.007 | 0.19 ± 0.038 |
| | (25 ± 8)% | | (116 ± 10)% | (107 ± 1)% |
| D20 | 1.0 ± 0.21 | NA | 0.82 ± 0.12 | 11.54 ± 2.81 |
| | (42 ± 9)% | | (104 ± 3)% | (35 ± 6)% |
| D21 | 1.09 ± 0.31 | NA | 0.33 ± 0.071 | 2.7 ± 0.042 |
| | (59 ± 12)% | | (92 ± 9)% | (82 ± 8)% |
| D22 | 0.43 ± 0.096 | NA | 0.57 ± 0.095 | 2.22 ± 0.093 |
| | (39 ± 6)% | | (107 ± 9)% | (80 ± 4)% |
| D23 | 0.019 ± 0.01 | NA | 2.03 ± 0.49 | 19.73 ± 4.43 |
| | (31 ± 2)% | | (104 ± 5)% | (51 ± 6)% |
| D24 | 0.57 ± 0.085 | NA | 1.07 ± 0.43 | 2.69 ± 0.93 |
| | (58 ± 2)% | | (53 ± 15)% | (46 ± 10)% |
| D25 | 0.34 ± 0.09 | NA | 0.32 ± 0.12 | 1.12 ± 0.17 |
| | (38 ± 3)% | | (122 ± 23)% | (102 ± 5)% |
| D26 | 1.13 ± 0.36 | NA | 0.24 ± 0.041 | 1.28 ± 0.28 |
| | (77 ± 7)% | | (111 ± 17)% | (189 ± 3)% |
| D27 | 1 ± 0.08 | NA | 1.14 ± 0.58 | 2.14 ± 0.36 |
| | (70 ± 4)% | | (91 ± 17)% | (92 ± 1)% |
| D28 | 0.23 ± 0.061 | NA | 0.29 ± 0.13 | 0.45 ± 0.18 |
| | (42 ± 5)% | | (115 ± 19)% | (104 ± 4)% |
| D29 | 0.31 ± 0.17 | NA | 0.31 ± 0.092 | 1.09 ± 0.3 |
| | (25 ± 4)% | | (105 ± 18)% | (107 ± 6)% |
| D30 | 1.71 ± 0.33 | NA | 0.19 ± 0.061 | 0.92 ± 0.12 |
| | (71 ± 1)% | | (118 ± 9)% | (104 ± 4)% |
| D31 | 0.16 ± 0.002 | NA | 0.10 ± 0.0035 | 0.43 ± 0.039 |
| | (73 ± 2)% | | (126 ± 18)% | (105 ± 1)% |
| D32 | NA | NA | NA | NA |
| D33 | NA | NA | 1.75 ± 0.37 | 5.5 ± 0.58 |
| | | | (156 ± 11)% | (112 ± 1)% |
| D34 | 1.07 ± 0.3 | 0.92 ± 0.29 | NA | 3.53 ± 1.23 |
| | (49 ± 8)% | (23 ± 3)% | | (41 ± 3)% |
| D35 | NA | NA | NA | 19.7 ± 7.63 |
| | | | | (84 ± 14)% |
| D36 | NA ( )% | NA | NA | 0.24 ± 0.04 |
| | | | | (100 ± 3)% |
| D37 | 0.085 ± 0.011 | NA | 0.14 ± 0.021 | 0.64 ± 0.033 |
| | (22 ± 4)% | | (139 ± 7)% | (107 ± 2)% |
| D38 | 2.43 ± 047 | NA | 0.33 ± 0.064 | 1.58 ± 0.17 |
| | (67 ± 1)% | | (138 ± 19)% | (104 ± 2)% |
| D39 | NA | NA | 0.014 ± 0.0014 | 0.12 ± 0.029 |
| | | | (139 ± 16)% | (109 ± 2)% |
| D40 | 0.26 ± 0.041 | NA | 0.12 ± 0.018 | 0.47 ± 0.057 |
| | (43 ± 1)% | | (138 ± 27)% | (109 ± 1)% |
| D41 | 2.41 ± 0.41 | NA | 21.45 ± 6.01 | NA |
| | (75 ± 2)% | | (65 ± 12)% | |
| E1 | NA | 0.32 ± 0.1 | NA | NA |
| | | (30 ± 7)% | | |
| E2 | 6.94 ± 0.20 | 5.16 ± 1.1 | NA | NA |
| | (54 ± 6)% | (24 ± 2)% | | |
| E3 | NA | 0.86 ± 0.12 | NA | NA |
| | | (29 ± 4)% | | |
| E4 | NA | NA | NA | NA |
| E5 | NA | 4.05 ± 1.52 | NA | NA |
| | | (41 ± 3)% | | |
| E6 | NA | 1.44 ± 0.27 | NA | NA |
| | | (35 ± 3)% | | |
| E7 | NA | 0.79 ± 0.26 | NA | NA |
| | | (29 ± 5)% | | |
| E8 | NA | NA | NA | NA |
| E9 | NA | NA | NA | NA |
| E10 | 1.20 ± 0.30 | NA | NA | NA |
| | (53 ± 2)% | | | |
| F1 | 0.044 ± 0.016 | NA | 0.066 ± 0.014 | 0.24 ± 0.016 |
| | (67 ± 1)% | | (93 ± 5)% | (102 ± 3)% |
| F2 | 7.68 ± 1.45 | NA | 3.62 ± 1.74 | 18.16 ± 11.06 |
| | (65 ± 4)% | | (101 ± 14)% | (56 ± 8)% |
| F3 | 0.12 ± 0.0028 | NA | 0.062 ± 0.0023 | 0.21 ± 0.049 |
| | (83 ± 2)% | | (89 ± 6)% | (104 ± 5)% |
| F4 | 1.64 ± 0.065 | NA | 0.29 ± 0.18 | 1.18 ± 0.21 |
| | (84 ± 1)% | | (101 ± 1)% | (104 ± 6)% |
| F5 | 0.14 ± 0.033 | NA | 0.18 ± 0.0071 | 0.48 ± 0.14 |
| | (40 ± 10)% | | (80 ± 5)% | (103 ± 5)% |
| F6 | 0.042 ± 0.011 | NA | 0.22 ± 0.035 | 0.67 ± 0.16 |
| | (61 ± 0.4)% | | (65 ± 17)% | (94 ± 5)% |
| F7 | 0.016 ± 0.003 | NA | NA | 0.12 ± 0.022 |
| | (94 ± 2)% | | | (88 ± 2)% |
| F8 | 0.021 ± 0.0041 | NA | NA | 0.023 ± 0.0027 |
| | (91 ± 2)% | | | (91 ± 4)% |
| F9 | 0.020 ± 0.0061 | NA | NA | 0.015 ± 0.00052 |
| | (92 ± 1)% | | | (88 ± 2)% |
| F10 | 0.0076 ± 0.0011 | 0.36 ± 0.16 | NA | 0.019 ± 0.0015 |
| | (90 ± 2)% | (9 ± 1)% | | (79 ± 2)% |
| G1 | 0.014 ± 0.003 | NA | NA | 0.074 ± 0.016 |
| | (95 ± 2)% | | | (84 ± 2)% |
| G2 | 1.93 ± 0.3 | NA | 0.48 ± 0.080 | 0.93 ± 0.11 |
| | (75 ± 5)% | | (115 ± 2)% | (108 ± 5)% |
| G3 | 1.08 ± 0.36 | NA | 0.82 ± 0.048 | 1.7 ± 0.27 |
| | (71 ± 1)% | | (111 ± 13)% | (105 ± 4)% |
| G4 | 0.10 ± 0.038 | NA | 0.074 ± 0.013 | 0.20 ± 0.0033 |
| | (74 ± 3)% | | (101 ± 9)% | (104 ± 3)% |
| G5 | 5.62 ± 0.49 | NA | 18.91 ± 3.74 | 7.05 ± 3.48 |
| | (57 ± 7)% | | (47 ± 6)% | (79 ± 9)% |
| G6 | 0.25 ± 0.048 | NA | 0.044 ± 0.014 | 0.15 ± 0.037 |
| | (87 ± 2)% | | (79 ± 5)% | (104 ± 3)% |
| G7 | 2.29 ± 0.83 | NA | 0.64 ± 0.008 | 1.31 ± 0.23 |
| | (81 ± 1)% | | (75 ± 17)% | (103 ± 8)% |
| G8 | 0.53 ± 0.075 | NA | 0.20 ± 0.039 | 0.64 ± 0.10 |
| | (57 ± 5)% | | (115 ± 2)% | (109 ± 6)% |
| G9 | 0.21 ± 0.012 | NA | 0.15 ± 0.04 | 0.40 ± 0.056 |
| | (62 ± 10)% | | (96 ± 6)% | (105 ± 4)% |
| G10 | 0.017 ± 0.0031 | 0.0031 ± 0.0017 | NA | 0.015 ± 0.0021 |
| | (94 ± 2)% | (10 ± 3)% | | (83 ± 6)% |
| G11 | 0.016 ± 0.0041 | 0.0077 ± 0.0012 | NA | 0.0069 ± 0.0015 |
| | (91 ± 3)% | (7 ± 1)% | | (83 ± 1)% |
| G12 | 0.0076 ± 0.0031 | 0.018 ± 0.0021 | NA | 0.0094 ± 0.00085 |
| | (92 ± 3)% | (17 ± 1)% | | (73 ± 2)% |
| H1 | 0.022 ± 0.004 | NA | NA | 0.30 ± 0.14 |
| | (88 ± 2)% | | | (76 ± 5)% |
| H2 | 0.04 ± 0.009 | NA | 0.0069 ± 0.0011 | 0.094 ± 0.0087 |
| | (94 ± 3)% | | (61 ± 4)% | (95 ± 2)% |
| H3 | 0.059 ± 0.014 | NA | NA | 0.70 ± 0.058 |
| | (86 ± 2)% | | | (74 ± 5)% |
| H4 | 0.12 ± 0.006 | 2.41 ± 0.62 | NA | NA |
| | (38 ± 5)% | (42 ± 8)% | | |
| H5 | 1.12 ± 1 | NA | NA | 0.44 ± 0.099 |
| | (64 ± 2)% | | | (32 ± 10)% |
| H6 | 0.028 ± 0.00036 | 0.48 ± 0.16 | NA | 0.40 ± 0.08 |
| | (86 ± 1)% | (22 ± 6)% | | (68 ± 2)% |
| H7 | 0.53 ± 0.017 | 3.67 ± 0.74 | NA | NA |
| | (14 ± 12)% | (36 ± 5)% | | |
| H8 | 0.053 ± 0.036 | 1.48 ± 0.17 | NA | NA |
| | (18 ± 1)% | (48 ± 1)% | | |
| H9 | 0.016 ± 0.0013 | NA | NA | NA |
| | (40 ± 8)% | | | |
| H10 | 1.31 ± 0.059 | NA | NA | NA |
| | (28 ± 8)% | | | |
| H11 | 0.12 ± 0.002 | 1.68 ± 0.34 | NA | NA |
| | (10 ± 0.4)% | (16 ± 6)% | | |
| H12 | 7.60 ± 2.06 | 4.28 ± 1.36 | NA | NA |
| | (75 ± 5)% | (44 ± 4)% | | |
| H13 | NA | NA | NA | NA |
| H14 | 0.33 ± 0.16 | 1.04 ± 0.092 | NA | NA |
| | (75 ± 5)% | (83 ± 4)% | | |
| H15 | 0.44 ± 0.3 | 4.07 ± 1.31 | NA | NA |
| | (54 ± 11)% | (86 ± 7)% | | |
| H16 | NA | 2.77 ± 0.47 | NA | NA |
| | | (32 ± 4)% | | |
| H17 | NA | 4.38 ± 0.08 | NA | NA |
| | | (26 ± 4)% | | |
| H18 | NA | 4.61 ± 1.18 | NA | NA |
| | | (43 ± 8)% | | |
| H19 | 0.070 ± 0.009 | NA | NA | 0.46 ± 0.056 |
| | (82 ± 2)% | | | (83 ± 5)% |
| H20 | 0.093 ± 0.005 | 0.24 ± 0.092 | NA | 1.44 ± 0.18 |
| | (75 ± 3)% | (45 ± 2)% | | (26 ± 3)% |
| I1 | 0.0062 ± 0.0001 | 0.01 ± 0.0001 | NA | 0.031 ± 0.0086 |
| | (89 ± 2)% | (8 ± 2)% | | (84 ± 6)% |
| I2 | 0.0072 ± 0.0001 | 0.049 ± 0.008 | NA | 0.032 ± 0.0034 |
| | (88 ± 2)% | (23 ± 19)% | | (78 ± 2)% |
| I3 | 0.0085 ± 0.0011 | 0.066 ± 0.013 | NA | 0.061 ± 0.011 |
| | (88 ± 1)% | (25 ± 3)% | | (66 ± 1)% |
| J1 | 0.0038 ± 0.0001 | 0.0014 ± 0.00037 | NA | 4.49 ± 0.21 |
| | (86 ± 3)% | (54 ± 11)% | | (48 ± 9)% |
| J2 | 0.026 ± 0.0081 | 0.008 ± 0.0014 | NA | 8.70 ± 0.28 |
| | (90 ± 2)% | (64 ± 3)% | | (31 ± 2)% |
| J3 | 0.030 ± 0.0011 | 0.0094 ± 0.0016 | NA | 12 ± 1.67 |
| | (91 ± 3)% | (58 ± 3)% | | (37 ± 3)% |
| K1 | 0.24 ± 0.03 | 0.67 ± 0.13 | NA | 1.52 ± 0.63 |
| | (80 ± 5)% | (40 ± 4)% | | (40 ± 5)% |
| K2 | 8.47 ± 0.78 | NA | NA | NA |
| | (38 ± 2)% | | | |
| K3 | NA | 3.39 ± 0.14 | NA | NA |
| | | (61 ± 4)% | | |
| K4 | 0.39 ± 0.19 | 1.45 ± 0.25 | NA | NA |
| | (24 ± 2)% | (103 ± 5)% | | |
| K5 | 10.7 ± 0.23 | 7.46 ± 1.11 | NA | NA |
| | (43 ± 2)% | (32 ± 2)% | | |
| K6 | 0.0147 ± 0.0026 | 0.021 ± 0.0035 | NA | NA |
| | (97 ± 3)% | (136 ± 26)% | | |
| K7 | 0.33 ± 0.071 | 0.39 ± 0.058 | NA | 10.7 ± 2.39 |
| | (92 ± 4)% | (68 ± 5)% | | (25 ± 5)% |
| K8 | 0.14 ± 0.068 | 0.19 ± 0.023 | NA | 1.45 ± 0.2 |
| | (70 ± 2)% | (21 ± 3)% | | (49 ± 5)% |
| L1 | NA | NA | NA | 5.44 ± 1.00 |
| | | | | (40 ± 5)% |
| L2 | NA | NA | NA | 0.70 ± 0.096 |
| | | | | (51 ± 4)% |
| M1 | 0.015 ± 0.003 | NA | 0.0011 ± 0.0005 | 0.0069 ± 0.0005 |
| | (93 ± 2)% | | (165 ± 4)5 | (105 ± 1)% |
| M2 | 0.019 ± 0.003 | NA | 0.0084 ± 0.004 | 0.052 ± 0.01 |
| | (88 ± 1)% | | (130 ± 7)% | (103 ± 1)% |
| M3 | 0.013 ± 0.003 | NA | 0.0034 ± 0.0003 | 0.033 ± 0.004 |
| | (77 ± 2)% | | (106 ± 7)% | (107 ± 1)% |
| M4 | 0.003 ± 0.0005 | NA | 0.001 ± 0.0002 | 0.006 ± 0.0008 |
| | (84 ± 5)% | | (142 ± 16)% | (112 ± 1)% |
| M5 | 0.25 ± 0.06 | Not tested | Not tested | Not tested |
| | (87 ± 3)% | | | |
| M6 | Not tested | NA | 0.011 ± 0.001 | 0.07 ± 0.01 |
| | | | (112 ± 9)% | (108 ± 2)% |
| M7 | Not tested | 0.013 ± 0.003 | NA | 0.13 ± 0.04 |
| | | (29 ± 4)% | | (76 ± 5)% |
| M8 | Not tested | NA | 0.009 ± 0.002 | 0.19 ± 0.07 |
| | | | (93 ± 2)% | (97 ± 7)% |
| M9 | 0.0078 ± 0.0001 | 0.013 ± 0.01 | NA | 0.12 ± 0.01 |
| | (91 ± 1)% | (69 ± 4)% | | (32 ± 1)% |
| M10 | 0.020 ± 0.005 | NA | 0.0044 ± 0.0015 | 0.023 ± 0.003 |
| | (92 ± 2)% | | (92 ± 6)% | (104 ± 1)% |
| M11 | 0.045 ± 0.013 | 0.0066 ± 0.0002 | NA | 65 ± 28 |
| | (94 ± 1)% | (92 ± 8)% | | (15 ± 1)% |
| M12 | 0.23 ± 0.1 | NA | 0.065 ± 0.039 | 0.32 ± 0.02 |
| | (90 ± 1)% | | (97 ± 10)% | (99 ± 2)% |

^{a}T0901317 at 10 μM as 100% inhibition and DMSO (0.3%) as 0% inhibition for the hPXR binding assay;
^{b}Rifampicin at 10 μM as 100% activation and DMSO (0.3%) as 0% activation for the cell-based hPXR agonistic assay;
^{c}SPA70 at 10 μM as 100% inhibition and DMSO (0.3%) as 0% inhibition for the cell-based hPXR inverse agonistic assay;
^{d}SPA70 at 10 μM with rifampicin (5 μM) as 100% inhibition and rifampicin alone (5 μM) as 0% inhibition for the cell-based hPXR antagonistic assay;
^{e}NT: not tested;
^{f}NA: no IC₅₀ value could be experimentally determined within the concentration range tested (highest concentration tested at 30 μM).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 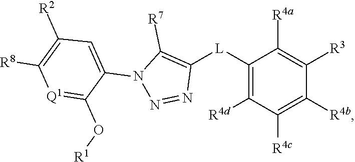
wherein L is selected from —NR¹⁰C(O)—, —N(R¹⁰)C(O)NR¹¹—, —C(O)NR¹⁰—, —SO₂NR¹⁰—, and —NR¹⁰SO₂—;
wherein R¹⁰ is selected from hydrogen and C1-C4 alkyl;
wherein R¹¹, when present, is selected from hydrogen and C1-C4 alkyl;
wherein Q¹ is selected from N and CH;
wherein R¹ is C1-C4 alkyl;
wherein R² is selected from halogen, C1-C4 alkyl, C1-C4 haloalkyl, C1-C4 alkoxy, and C1-C4 haloalkoxy;
wherein R³ is hydrogen, halogen, C1-C8 alkyl, C1-C8 haloalkyl, C1-C8 alkoxy, C1-C8 haloalkoxy, —CO₂(C1-C4 alkyl), and —C(O)Cy²;
wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy¹;
wherein Cy³, when present, is a C2-C5 heterocycloalkyl substituted with 0 or 1 group selected from C1-C4 alkyl and —C(O)(C1-C4 alkyl); and
wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)˜OR¹⁴, —NHR¹⁵, —B(OR¹⁶)₂, —OCy¹, and Cy¹;
wherein n, when present, is selected from 1, 2, 3, 4, and 5;
wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula: 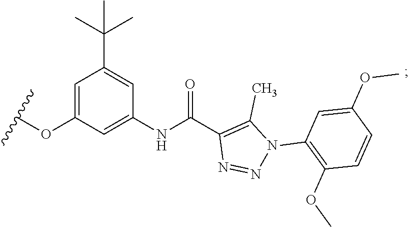
wherein R¹⁴, when present, is selected from hydrogen and C1-C4 alkyl;
wherein R¹⁵, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl);
wherein each occurrence of R¹⁶, when present, is independently selected from hydrogen and C1-C4 alkyl,
or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups;
wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl); and
wherein R⁸ is selected from hydrogen, halogen, and C1-C4 alkoxy;
wherein R⁷ is selected from hydrogen and C1-C4 alkyl;
provided that when L is —C(O)NR¹⁰—, then R² is selected from C1-C4 haloalkyl, C1-C4 alkoxy, and C1-C4 haloalkoxy, and R³ is C1-C8 alkyl, —CO₂(C1-C4 alkyl), or —C(O)Cy²,
or a pharmaceutically acceptable salt thereof.

2. The compound of claim 1, wherein L is selected from —C(O)NR¹⁰— and —SO₂NR¹⁰—.

3. (canceled)

4. The compound of claim 1, wherein Q¹ is CH.

5. (canceled)

6. The compound of claim 1, wherein R² is C1-C4 alkoxy.

7-9. (canceled)

10. The compound of claim 1, wherein R³ is C1-C4 alkyl.

11. (canceled)

12. The compound of claim 1, wherein R³ is —CO₂(C1-C4 alkyl) or —C(O)Cy².

13-17. (canceled)

18. The compound of claim 1, wherein R^{4d} is selected from halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)˜OR¹⁴, —NHR¹⁵, —B(OR¹⁶)₂, —OCy¹, and Cy¹.

19-26. (canceled)

27. The compound of claim 1, wherein the compound has a structure represented by a formula: 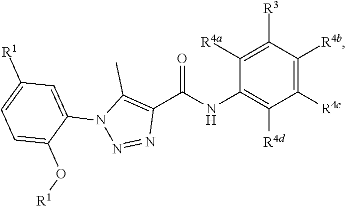
or a pharmaceutically acceptable salt thereof.

28. The compound of claim 1, wherein the compound has a structure represented by a formula: 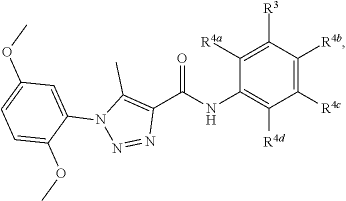
or a pharmaceutically acceptable salt thereof.

29-32. (canceled)

33. The compound of claim 1, wherein the compound has a structure represented by a formula: 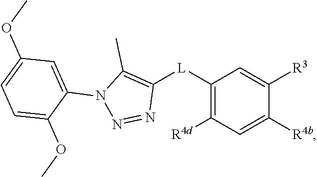
or a pharmaceutically acceptable salt thereof.

34-38. (canceled)

39. The compound of claim 33, wherein the compound is selected from: 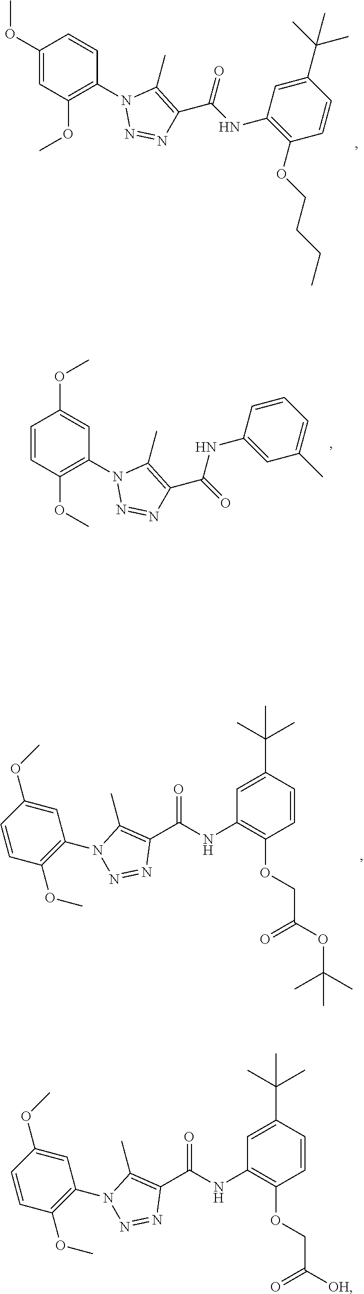 [SMILES: CCCCOc1ccc(C(C)(C)C)cc1NC(=O)c1nnn(-c2ccc(OC)cc2OC)c1C ; COc1ccc(OC)c(-n2nnc(C(=O)Nc3cccc(C)c3)c2C)c1 ; COc1ccc(OC)c(-n2nnc(C(=O)Nc3cc(C(C)(C)C)ccc3OCC(=O)OC(C)(C)C)c2C)c1 ; COc1ccc(OC)c(-n2nnc(C(=O)Nc3cc(C(C)(C)C)ccc3OCC(=O)O)c2C)c1] 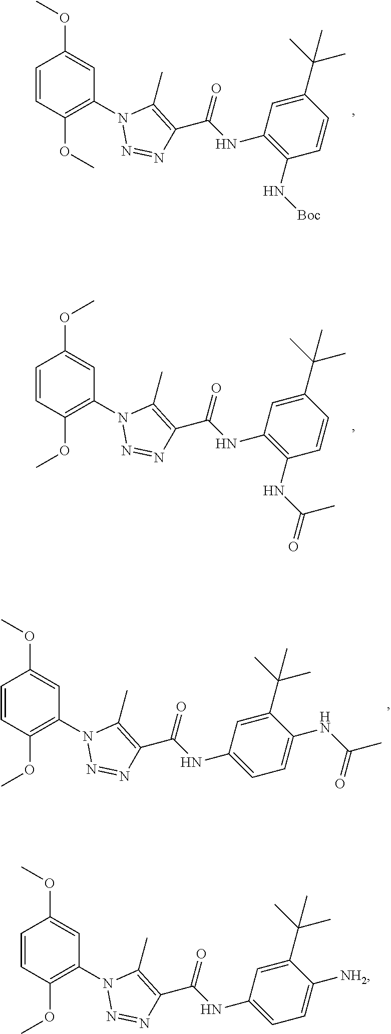 [SMILES: COc1ccc(OC)c(-n2nnc(C(=O)Nc3cc(C(C)(C)C)ccc3NC(=O)OC(C)(C)C)c2C)c1 ; COc1ccc(OC)c(-n2nnc(C(=O)Nc3cc(C(C)(C)C)ccc3NC(C)=O)c2C)c1 ; COc1ccc(OC)c(-n2nnc(C(=O)Nc3ccc(NC(C)=O)c(C(C)(C)C)c3)c2C)c1 ; COc1ccc(OC)c(-n2nnc(C(=O)Nc3ccc(N)c(C(C)(C)C)c3)c2C)c1] 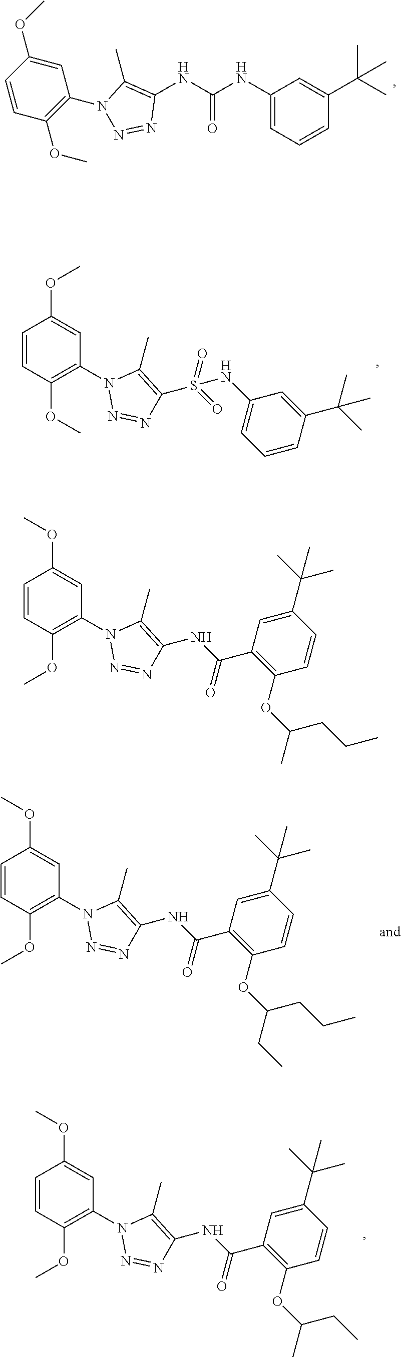 [SMILES: COc1ccc(OC)c(-n2nnc(NC(=O)Nc3cccc(C(C)(C)C)c3)c2C)c1 ; COc1ccc(OC)c(-n2nnc(S(=O)(=O)Nc3cccc(C(C)(C)C)c3)c2C)c1 ; CCCC(C)Oc1ccc(C(C)(C)C)cc1C(=O)Nc1nnn(-c2cc(OC)ccc2OC)c1C ; CCCC(CC)Oc1ccc(C(C)(C)C)cc1C(=O)Nc1nnn(-c2cc(OC)ccc2OC)c1C ; CCC(C)Oc1ccc(C(C)(C)C)cc1C(=O)Nc1nnn(-c2cc(OC)ccc2OC)c1C]
or a pharmaceutically acceptable salt thereof.

40. The compound of claim 1, wherein the compound has a structure represented by a formula: 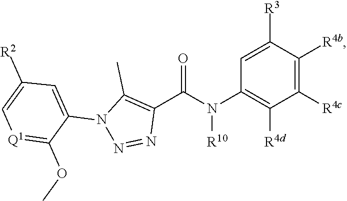
or a pharmaceutically acceptable salt thereof.

41-45. (canceled)

46. The compound of claim 40, wherein the compound is selected from: 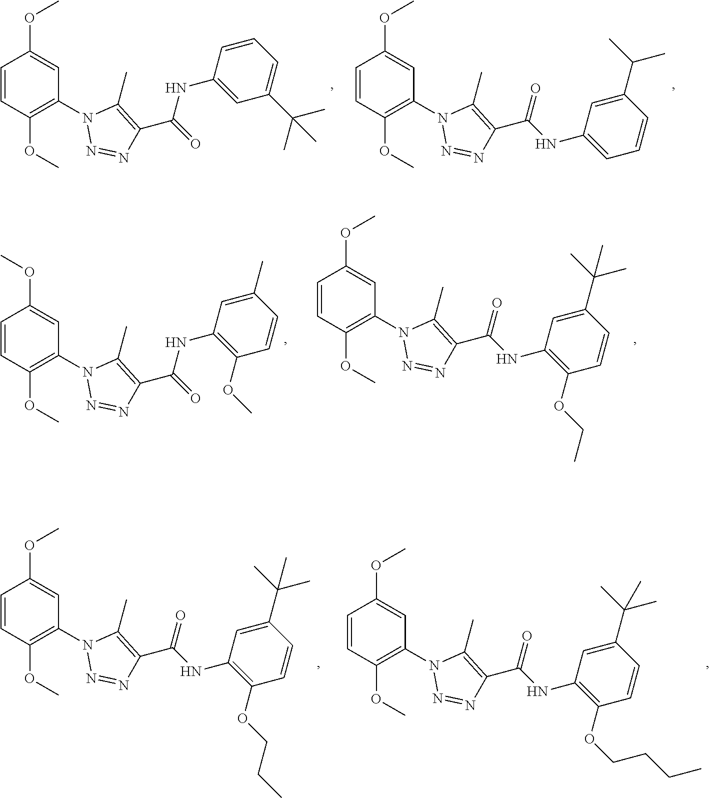 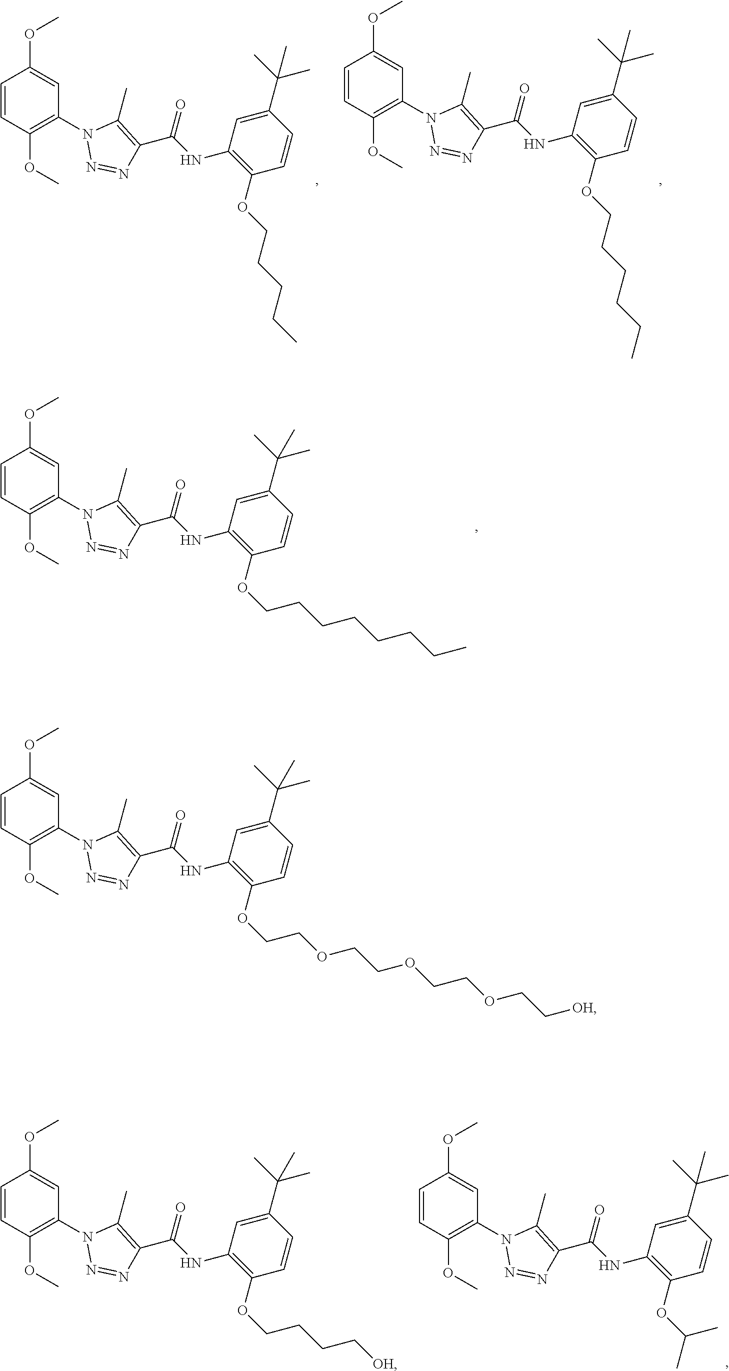 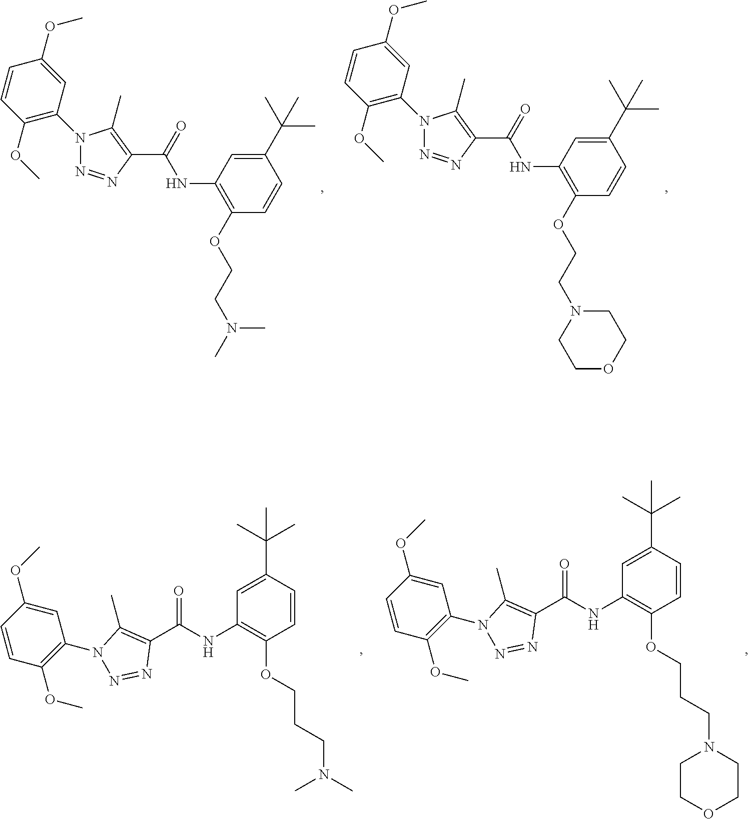 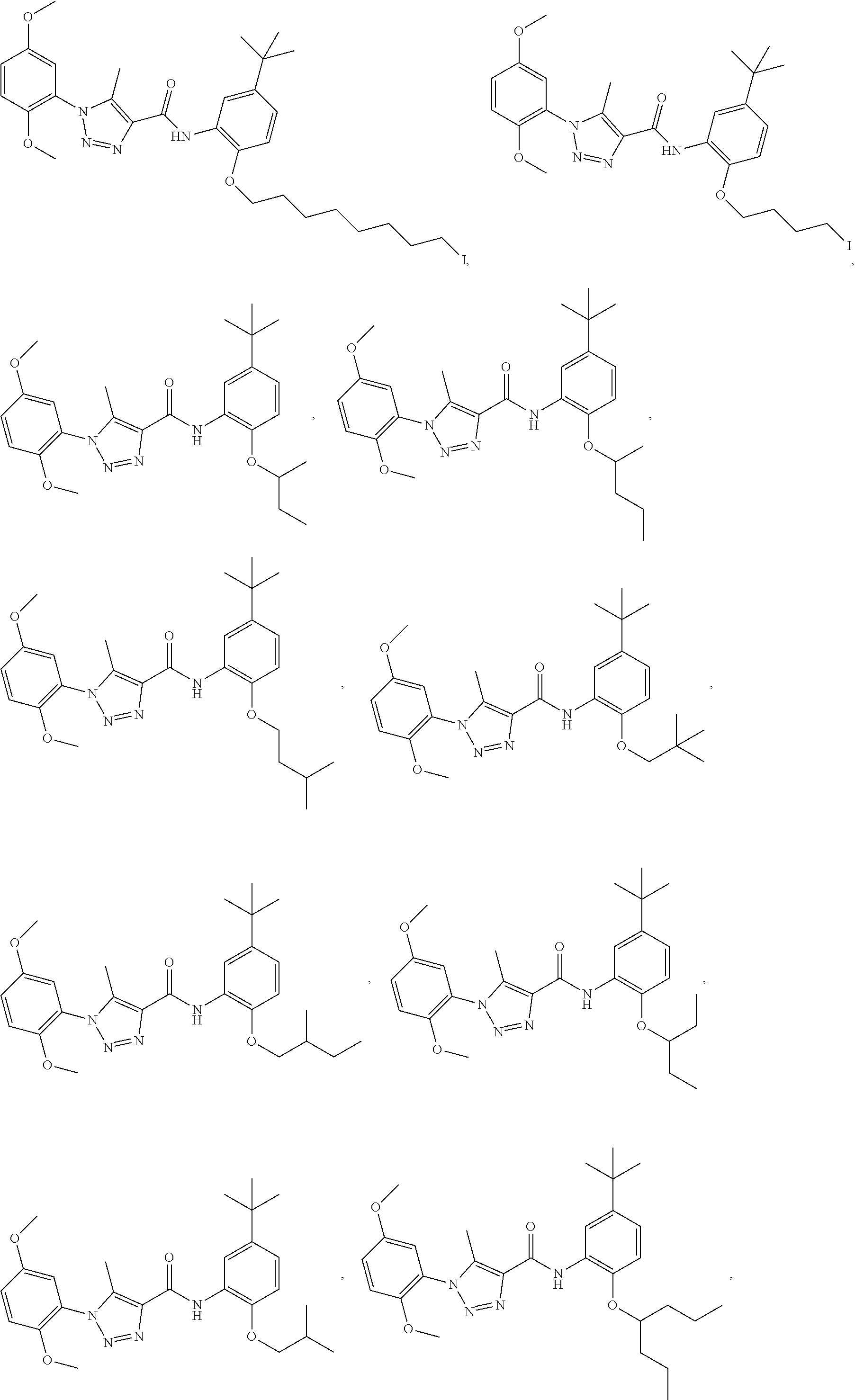 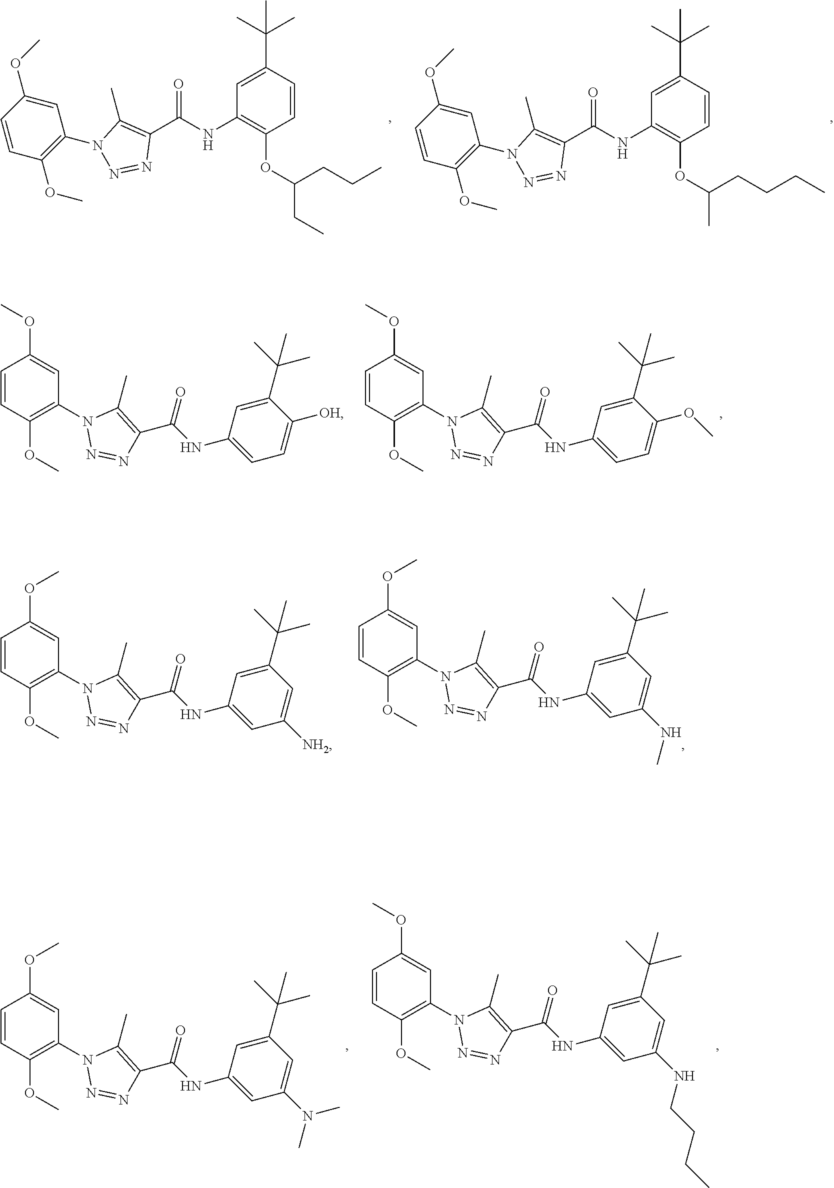 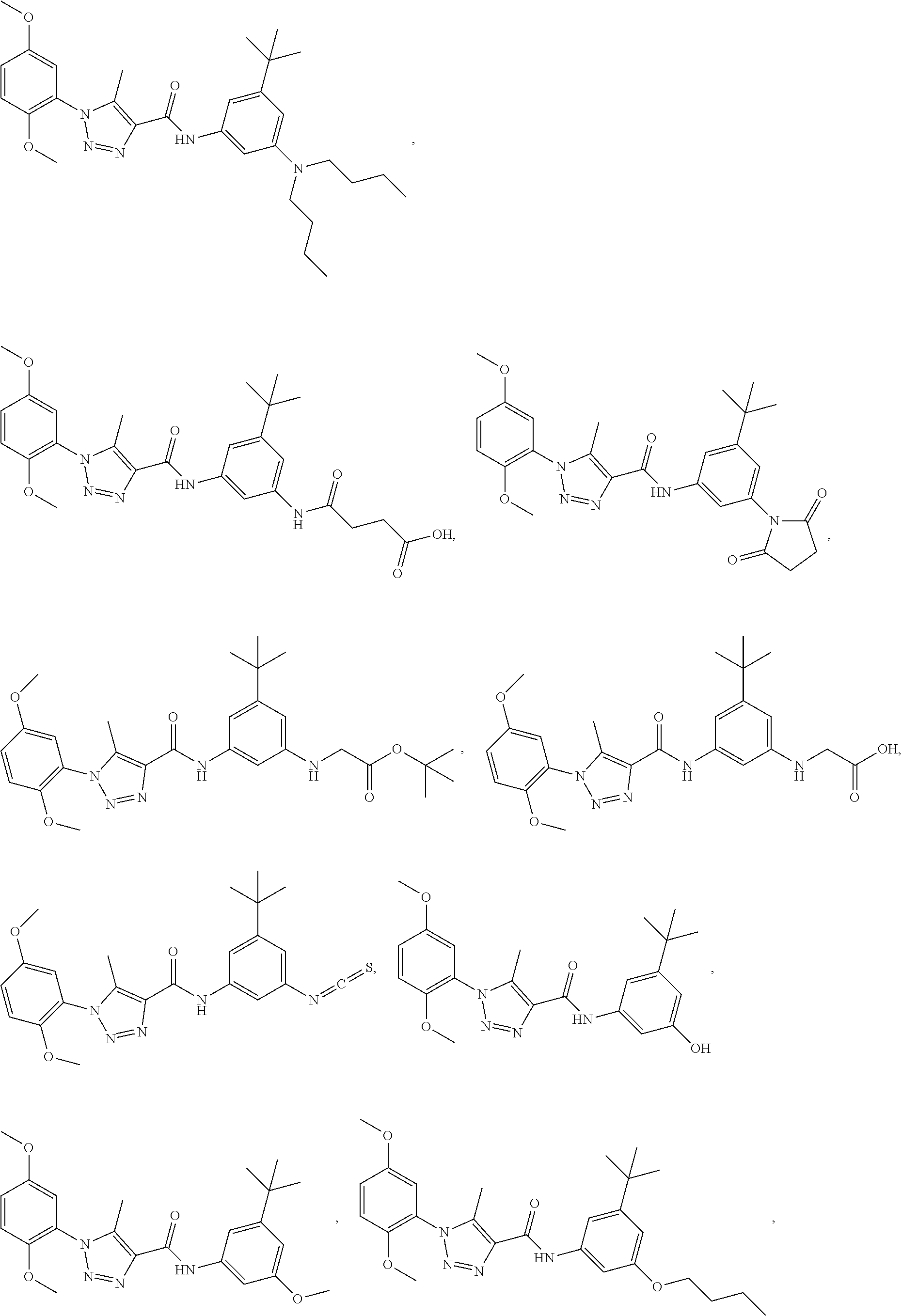 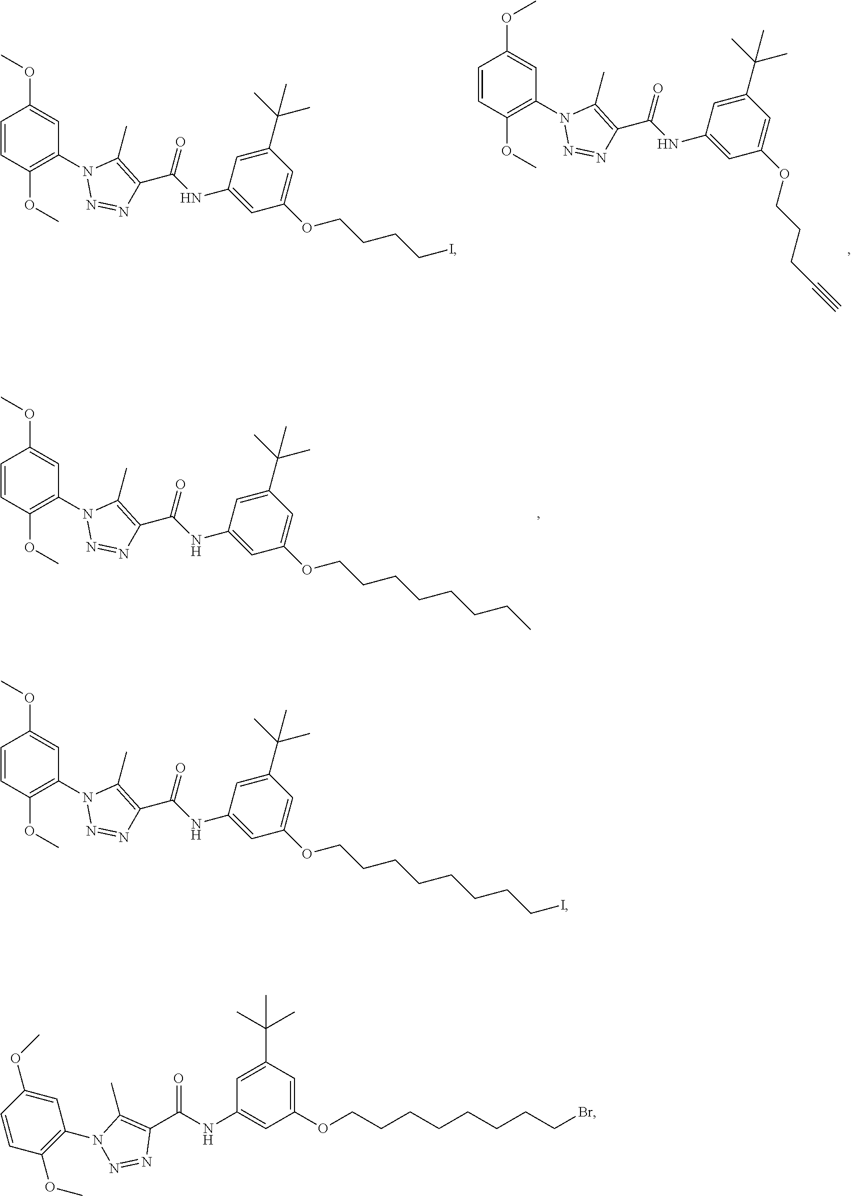 [SMILES: COc1ccc(OC)c(-n2nnc(C(=O)Nc3cc(OCCCCI)cc(C(C)(C)C)c3)c2C)c1 ; C#CCCCOc1cc(NC(=O)c2nnn(-c3cc(OC)ccc3OC)c2C)cc(C(C)(C)C)c1 ; CCCCCCCCOc1cc(NC(=O)c2nnn(-c3cc(OC)ccc3OC)c2C)cc(C(C)(C)C)c1 ; COc1ccc(OC)c(-n2nnc(C(=O)Nc3cc(OCCCCCCCCI)cc(C(C)(C)C)c3)c2C)c1 ; COc1ccc(OC)c(-n2nnc(C(=O)Nc3cc(OCCCCCCCCBr)cc(C(C)(C)C)c3)c2C)c1] 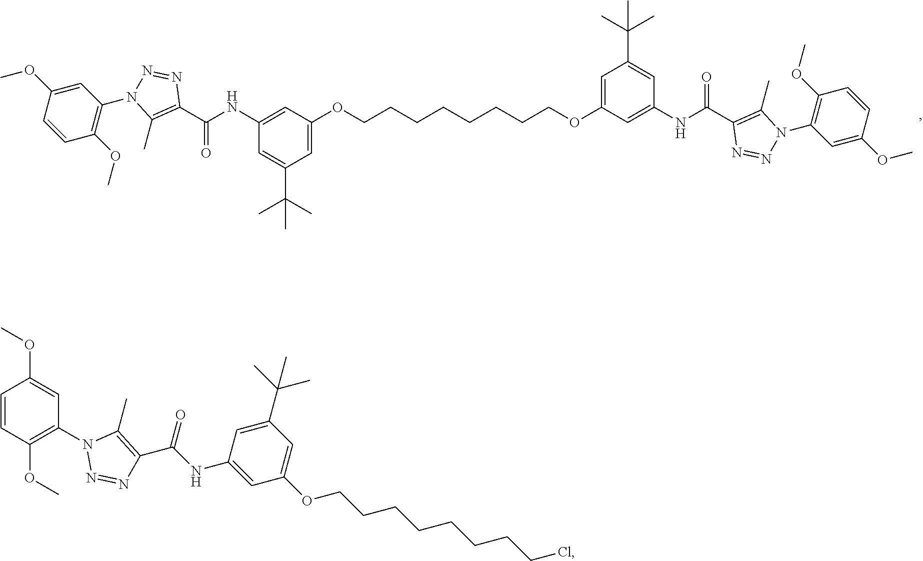 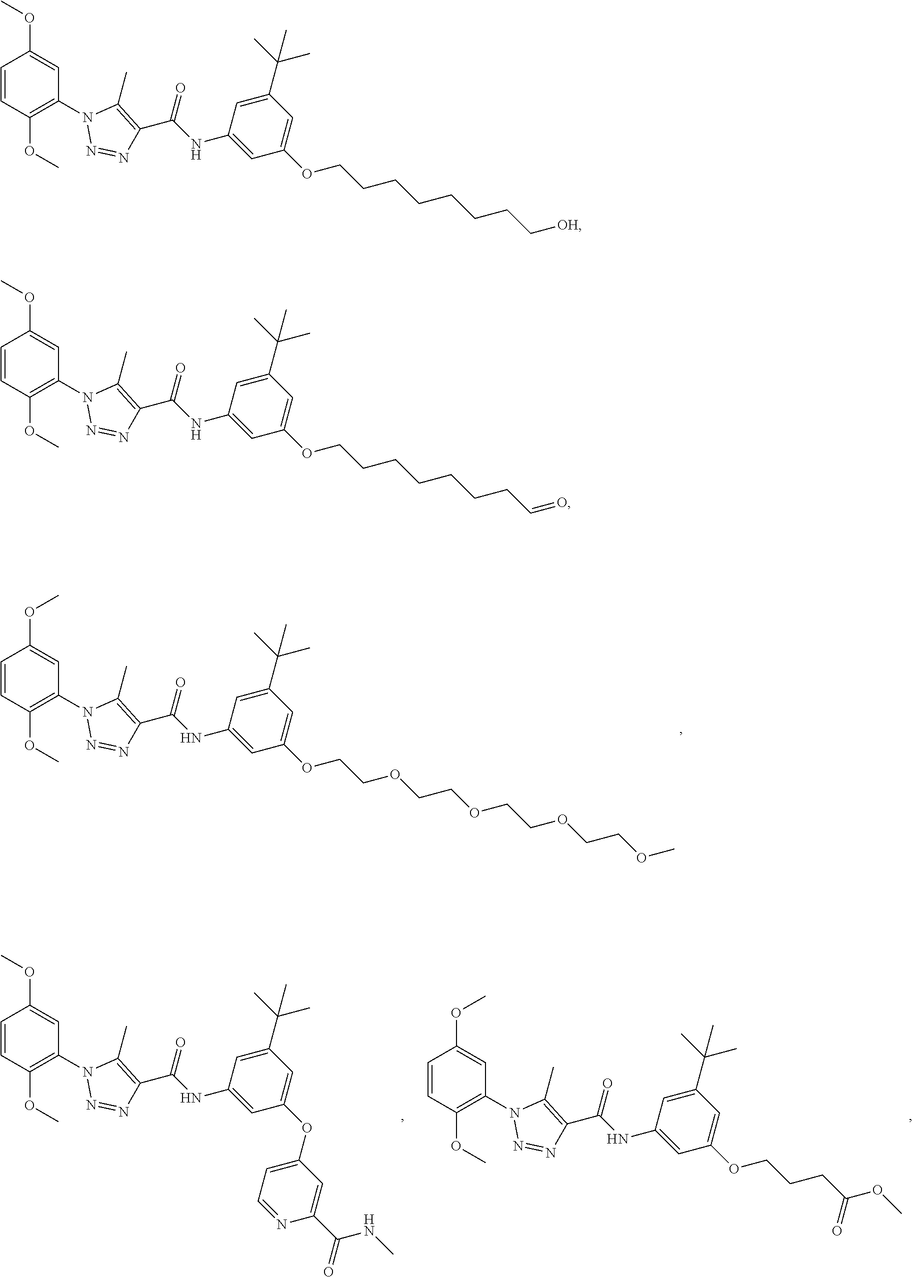 [SMILES: COc1ccc(OC)c(-n2nnc(C(=O)Nc3cc(OCCCCCCCCO)cc(C(C)(C)C)c3)c2C)c1 ; COc1ccc(OC)c(-n2nnc(C(=O)Nc3cc(OCCCCCCCC=O)cc(C(C)(C)C)c3)c2C)c1 ; COCCOCCOCCOCCOc1cc(NC(=O)c2nnn(-c3cc(OC)ccc3OC)c2C)cc(C(C)(C)C)c1 ; CNC(=O)c1cc(Oc2cc(NC(=O)c3nnn(-c4cc(OC)ccc4OC)c3C)cc(C(C)(C)C)c2)ccn1 ; COC(=O)CCCOc1cc(NC(=O)c2nnn(-c3cc(OC)ccc3OC)c2C)cc(C(C)(C)C)c1] 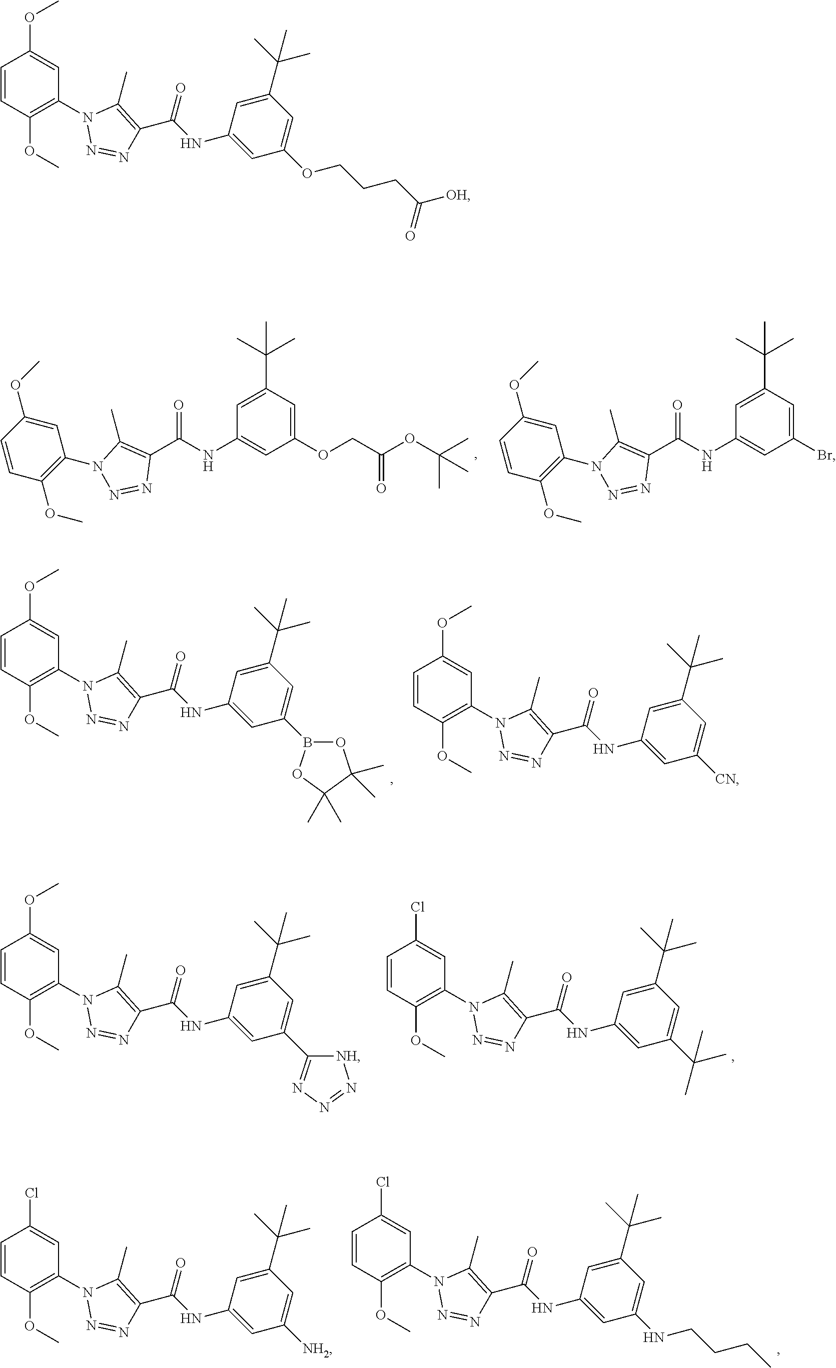 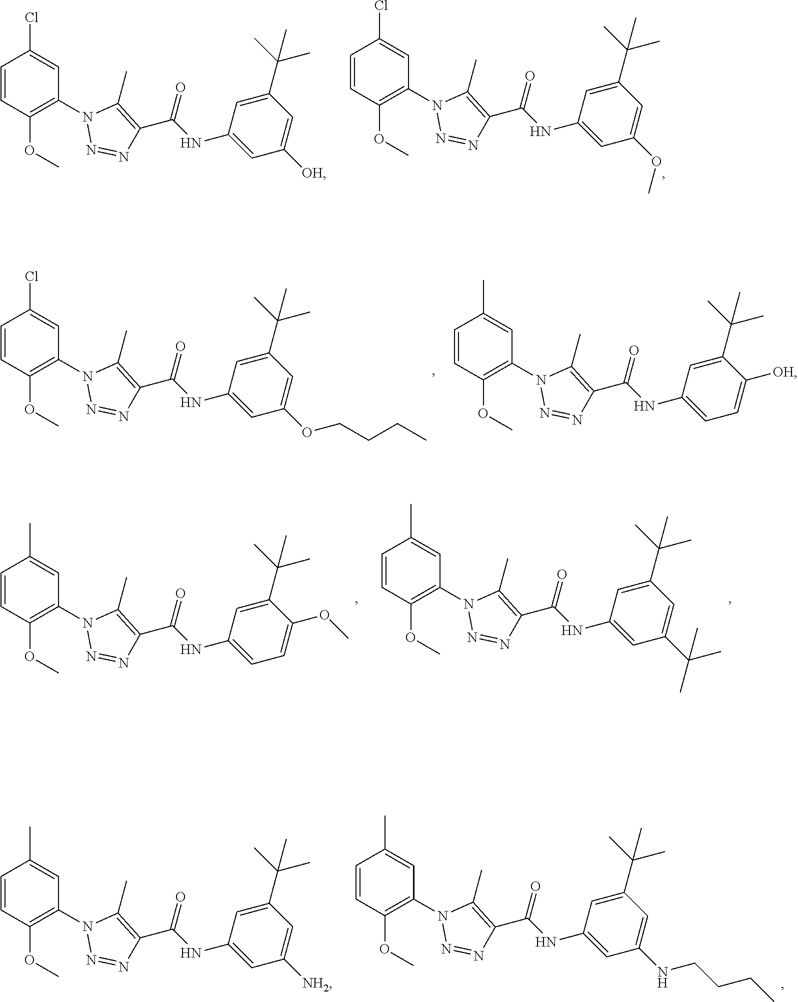 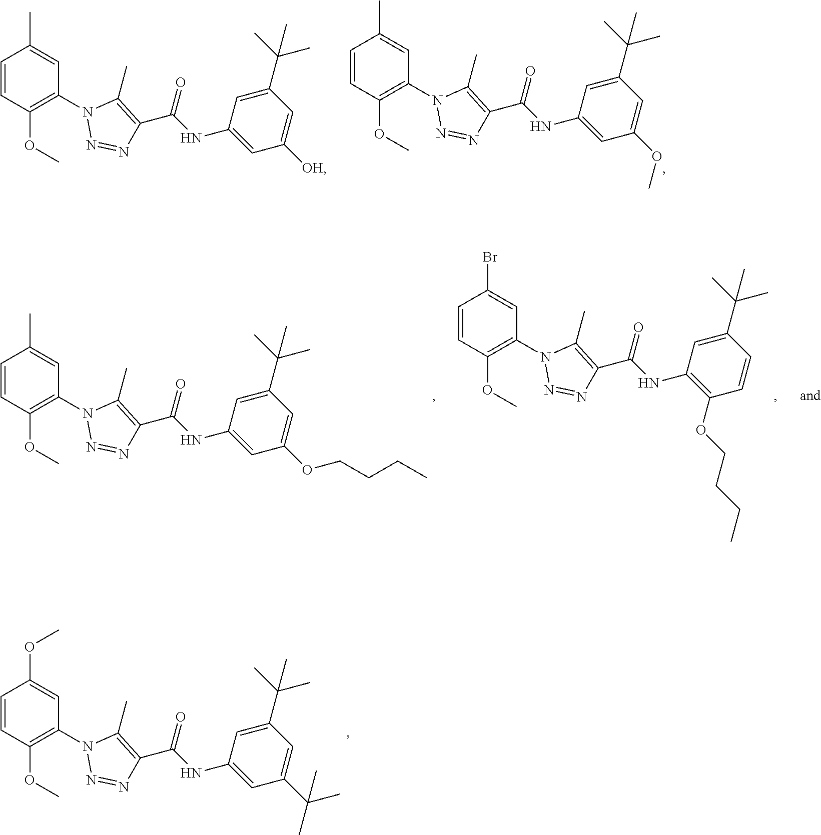
or a pharmaceutically acceptable salt thereof.

47. The compound of claim 40, wherein the compound is selected from: 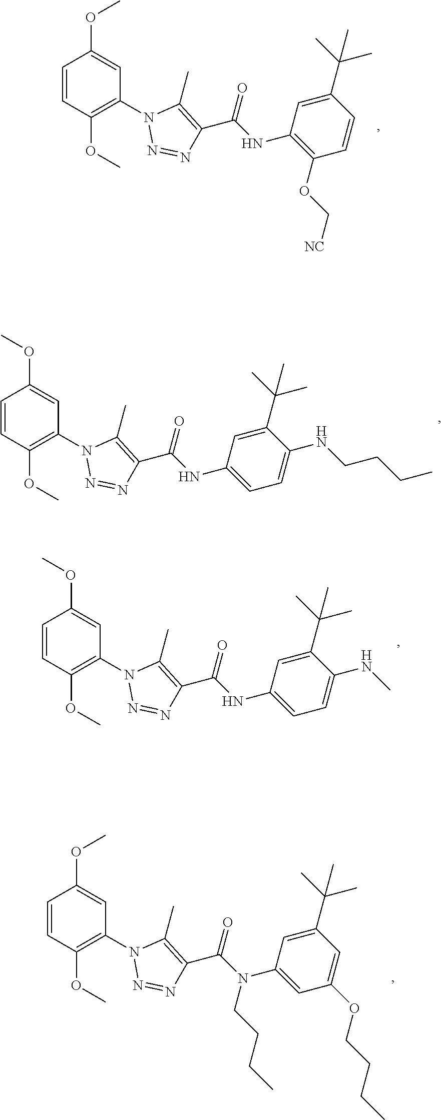 [SMILES: COc1ccc(OC)c(-n2nnc(C(=O)Nc3cc(C(C)(C)C)ccc3OCC#N)c2C)c1 ; CCCCNc1ccc(NC(=O)c2nnn(-c3cc(OC)ccc3OC)c2C)cc1C(C)(C)C ; CNc1ccc(NC(=O)c2nnn(-c3cc(OC)ccc3OC)c2C)cc1C(C)(C)C ; CCCCOc1cc(N(CCCC)C(=O)c2nnn(-c3cc(OC)ccc3OC)c2C)cc(C(C)(C)C)c1] 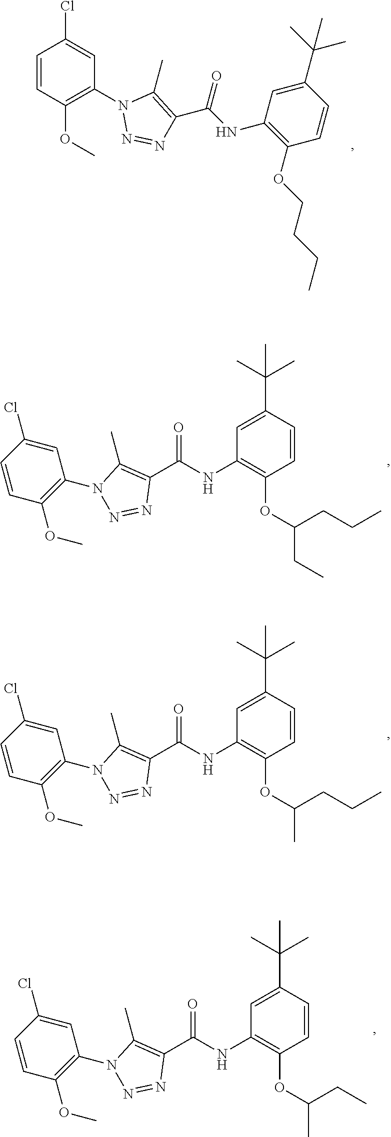 [SMILES: CCCCOc1ccc(C(C)(C)C)cc1NC(=O)c1nnn(-c2cc(Cl)ccc2OC)c1C ; CCCC(CC)Oc1ccc(C(C)(C)C)cc1NC(=O)c1nnn(-c2cc(Cl)ccc2OC)c1C ; CCCC(C)Oc1ccc(C(C)(C)C)cc1NC(=O)c1nnn(-c2cc(Cl)ccc2OC)c1C ; CCC(C)Oc1ccc(C(C)(C)C)cc1NC(=O)c1nnn(-c2cc(Cl)ccc2OC)c1C] 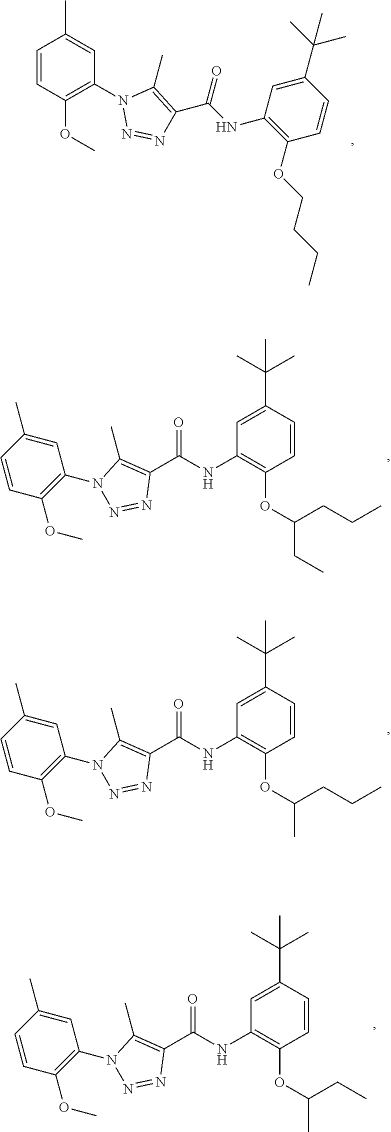 [SMILES: CCCCOc1ccc(C(C)(C)C)cc1NC(=O)c1nnn(-c2cc(C)ccc2OC)c1C ; CCCC(CC)Oc1ccc(C(C)(C)C)cc1NC(=O)c1nnn(-c2cc(C)ccc2OC)c1C ; CCCC(C)Oc1ccc(C(C)(C)C)cc1NC(=O)c1nnn(-c2cc(C)ccc2OC)c1C ; CCC(C)Oc1ccc(C(C)(C)C)cc1NC(=O)c1nnn(-c2cc(C)ccc2OC)c1C] 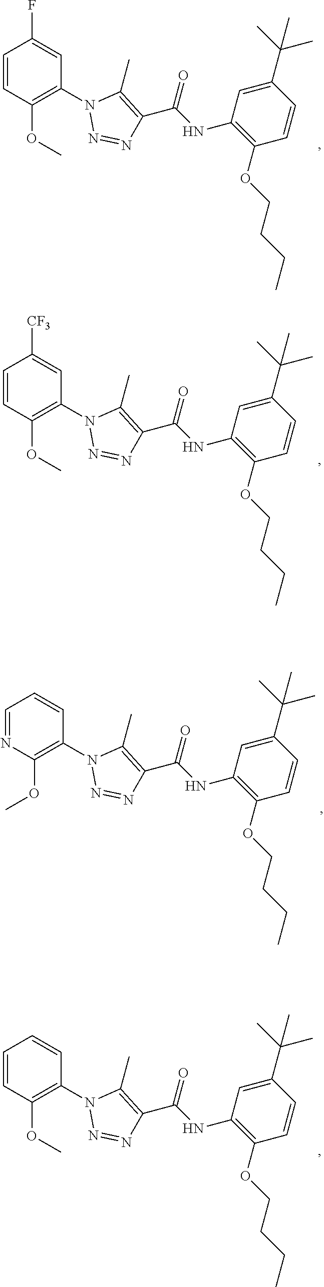 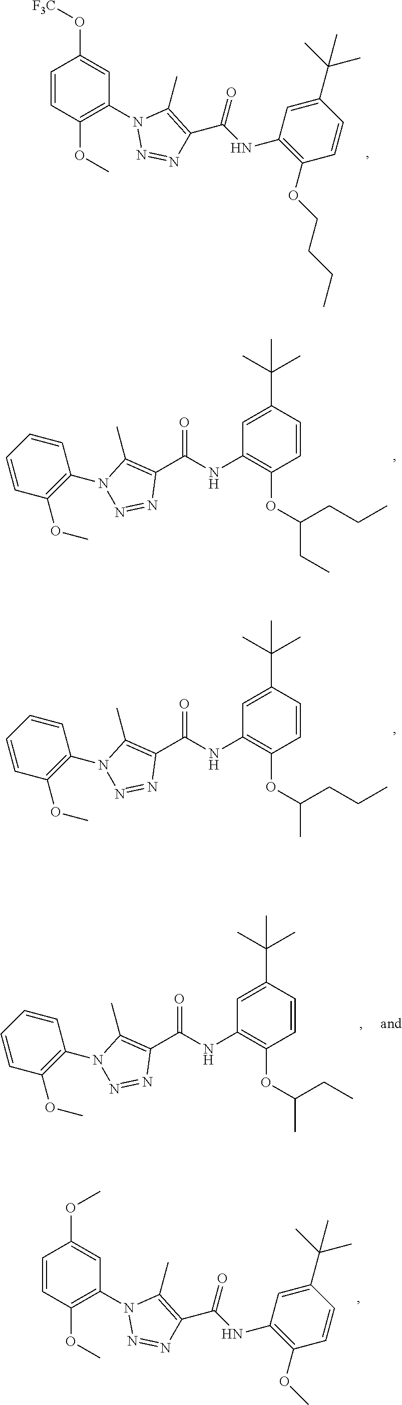 [SMILES: CCCCOc1ccc(C(C)(C)C)cc1NC(=O)c1nnn(-c2cc(OC(F)(F)F)ccc2OC)c1C ; CCCC(CC)Oc1ccc(C(C)(C)C)cc1NC(=O)c1nnn(-c2ccccc2OC)c1C ; CCCC(C)Oc1ccc(C(C)(C)C)cc1NC(=O)c1nnn(-c2ccccc2OC)c1C ; CCC(C)Oc1ccc(C(C)(C)C)cc1NC(=O)c1nnn(-c2ccccc2OC)c1C ; COc1ccc(OC)c(-n2nnc(C(=O)Nc3cc(C(C)(C)C)ccc3OC)c2C)c1]
or a pharmaceutically acceptable salt thereof.

48. The compound of claim 1, wherein the compound is selected from: 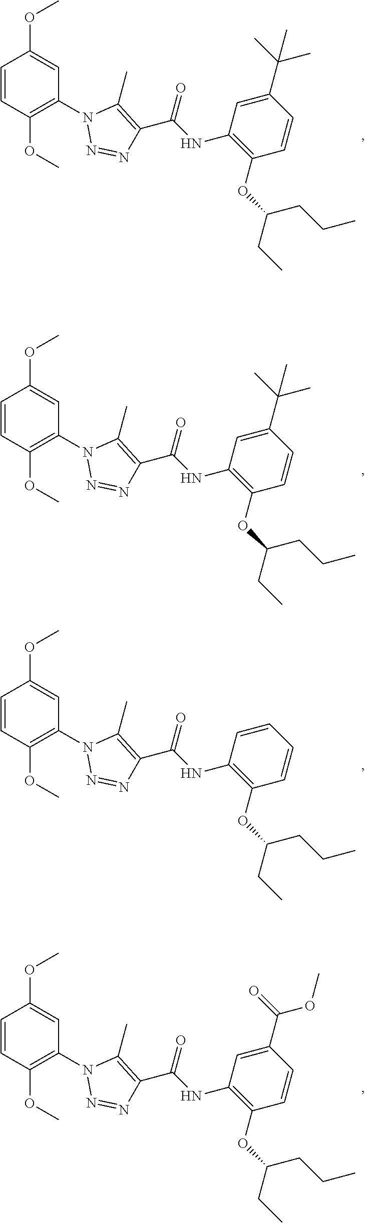 [SMILES: CCC[C@H](CC)Oc1ccc(C(C)(C)C)cc1NC(=O)c1nnn(-c2cc(OC)ccc2OC)c1C ; CCC[C@@H](CC)Oc1ccc(C(C)(C)C)cc1NC(=O)c1nnn(-c2cc(OC)ccc2OC)c1C ; CCC[C@H](CC)Oc1ccccc1NC(=O)c1nnn(-c2cc(OC)ccc2OC)c1C ; CCC[C@H](CC)Oc1ccc(C(=O)OC)cc1NC(=O)c1nnn(-c2cc(OC)ccc2OC)c1C] 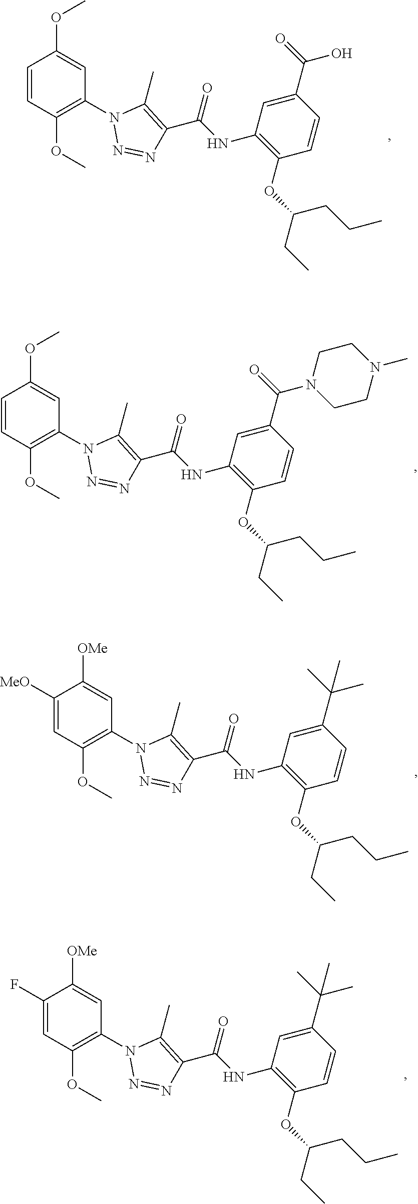 [SMILES: CCC[C@H](CC)Oc1ccc(C(=O)O)cc1NC(=O)c1nnn(-c2cc(OC)ccc2OC)c1C ; CCC[C@H](CC)Oc1ccc(C(=O)N2CCN(C)CC2)cc1NC(=O)c1nnn(-c2cc(OC)ccc2OC)c1C ; CCC[C@H](CC)Oc1ccc(C(C)(C)C)cc1NC(=O)c1nnn(-c2cc(OC)c(OC)cc2OC)c1C ; CCC[C@H](CC)Oc1ccc(C(C)(C)C)cc1NC(=O)c1nnn(-c2cc(OC)c(F)cc2OC)c1C] 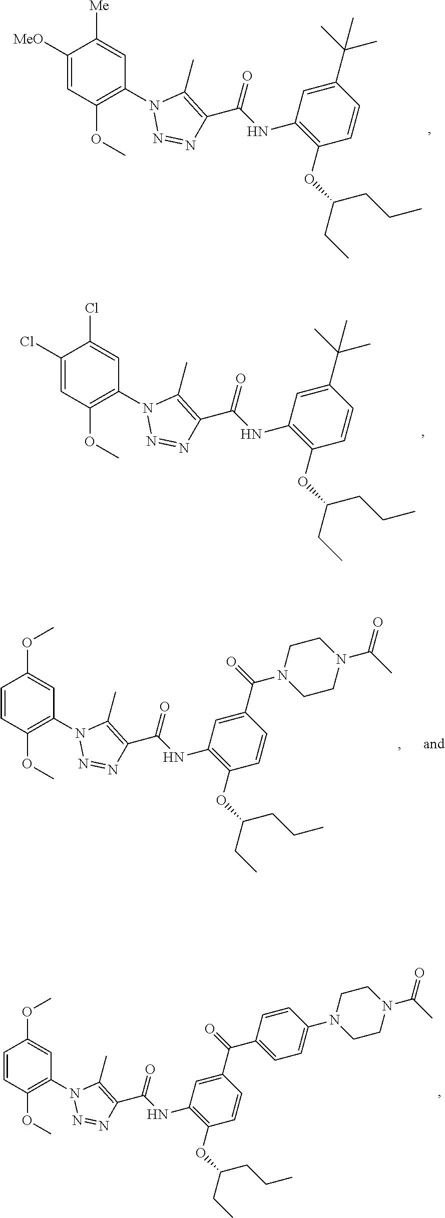 [SMILES: CCC[C@H](CC)Oc1ccc(C(C)(C)C)cc1NC(=O)c1nnn(-c2cc(C)c(OC)cc2OC)c1C ; CCC[C@H](CC)Oc1ccc(C(C)(C)C)cc1NC(=O)c1nnn(-c2cc(Cl)c(Cl)cc2OC)c1C ; CCC[C@H](CC)Oc1ccc(C(=O)N2CCN(C(C)=O)CC2)cc1NC(=O)c1nnn(-c2cc(OC)ccc2OC)c1C ; CCC[C@H](CC)Oc1ccc(C(=O)c2ccc(N3CCN(C(C)=O)CC3)cc2)cc1NC(=O)c1nnn(-c2cc(OC)ccc2OC)c1C]
or a pharmaceutically acceptable salt thereof.

49. (canceled)

50. A pharmaceutical composition comprising a therapeutically effective amount of the compound of claim 1 or a pharmaceutically acceptable salt thereof, and a pharmaceutically acceptable carrier.

51-66. (canceled)

67. A compound selected from: 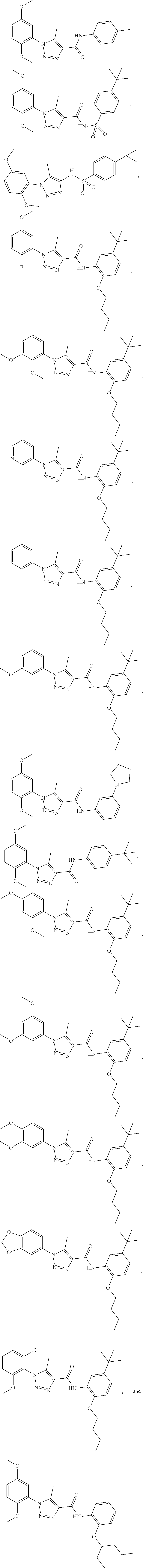
or a pharmaceutically acceptable salt thereof.

68. A pharmaceutical composition comprising a therapeutically effective amount of the compound of claim 67 or a pharmaceutically acceptable salt thereof, and a pharmaceutically acceptable carrier.

69-70. (canceled)

71. A method for decreasing an adverse drug reaction in a subject in need thereof, the method comprising administering an effective amount of a compound having a structure represented by a formula: 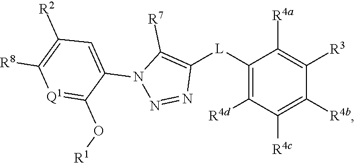
wherein L is selected from —NR¹⁰C(O)—, —N(R¹⁰)C(O)NR¹¹—, —C(O)NR¹⁰—, —SO₂NR¹⁰—, and —NR¹⁰SO₂—;
wherein R¹⁰ is selected from hydrogen and C1-C4 alkyl;
wherein R¹¹, when present, is selected from hydrogen and C1-C4 alkyl;
wherein Q¹ is selected from N and CH;
wherein R¹ is C1-C4 alkyl;
wherein R² is selected from C1-C4 haloalkyl, C1-C4 alkoxy, and C1-C4 haloalkoxy;
wherein R³ is selected from C1-C8 alkyl, —CO₂(C1-C4 alkyl), and —C(O)Cy²;
wherein Cy², when present, is selected from is selected from a C2-C5 heterocycloalkyl, a C6 aryl, and a C2-C5 heteroaryl, and is substituted with 0, 1, 2, or 3 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 aminoalkyl, —C(O)(C1-C4 alkyl), and Cy¹;
wherein Cy³, when present, is a C2-C5 heterocycloalkyl substituted with 0 or 1 group selected from C1-C4 alkyl and —C(O)(C1-C4 alkyl);
wherein each of R^{4a}, R^{4b}, R^{4c}, and R^{4d} is independently selected from hydrogen, halogen, —CN, —NH₂, —OH, —NO₂, —N═C═S, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —O—(C1-C8 alkyl)-R¹³, —(OCH₂CH₂)˜OR¹⁴, —NHR¹⁵, —B(OR¹⁶)₂, —OCy¹, and Cy¹;
wherein n, when present, is selected from 1, 2, 3, 4, and 5;
wherein R¹³, when present, is selected from halogen, —CN, —NH₂, —OH, —C≡CH, —CHO, —CO₂H, —CO₂(C1-C4 alkyl), C1-C4 alkylamino, (C1-C4)(C1-C4) dialkylamino, unsubstituted morpholine, and a structure represented by a formula: 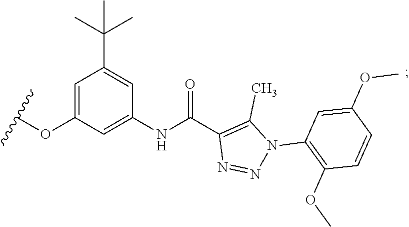
wherein R¹⁴, when present, is selected from hydrogen and C1-C4 alkyl;
wherein R¹¹, when present, is selected from —(C1-C4 alkyl)CO₂H, —(C1-C4 alkyl)CO₂(C1-C4 alkyl), —C(O)(C1-C4 alkyl), —CO₂(C1-C4 alkyl), —C(O)(C1-C4 alkyl)CO₂H, and —C(O)(C1-C4 alkyl)CO₂(C1-C4 alkyl);
wherein each occurrence of R¹⁶, when present, is independently selected from hydrogen and C1-C4 alkyl,
or wherein each occurrence of R¹⁶, when present, is covalently bonded and, together with the intermediate atoms, comprise a 5- or 6-membered heterocycloalkyl substituted with 0, 1, 2, 3, or 4 C1-C4 alkyl groups;
wherein Cy¹, when present, is selected from a C2-C5 heterocycloalkyl and a C2-C5 heteroaryl, and is substituted with 1, 2, 3, or 4 groups independently selected from halogen, ═O, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, C1-C8 alkylamino, —CONH₂, —CONH(C1-C4 alkyl), and —CON(C1-C4 alkyl)(C1-C4 alkyl);
wherein R⁷ is selected from hydrogen and C1-C4 alkyl;
wherein R⁸ is selected from hydrogen and halogen; and
wherein R¹⁰ is selected from hydrogen and C1-C4 alkyl,
or a pharmaceutically acceptable salt thereof.

72-75. (canceled)

76. The method of claim 71, wherein decreasing an adverse drug reaction is associated with the subject receiving treatment for a disorder of uncontrolled cellular proliferation.

77-156. (canceled)
